(19) United States
(12) Patent Application Publication
(10) Pub. No.: US 2024/0158468 A1
(43) Pub. Date: May 16, 2024

(54) TGF-BETA SUPERFAMILY TYPE I AND TYPE II RECEPTOR HETEROMULTIMERS AND USES THEREOF

(71) Applicant: Acceleron Pharma Inc., Cambridge, MA (US)

(72) Inventors: Ravindra Kumar, Acton, MA (US); Asya Grinberg, Lexington, MA (US); Dianne Sako, Medford, MA (US); Robert Scott Pearsall, North Reading, MA (US); Roselyne Castonguay, Watertown, MA (US)

(21) Appl. No.: 18/236,082

(22) Filed: Aug. 21, 2023

Related U.S. Application Data
(63) Continuation of application No. 16/951,192, filed on Nov. 18, 2020, now abandoned.
Application No. 16/951,192 is a continuation of application No. 16/251,325, filed on Jan. 18, 2019, now Pat. No. 10,906,958.
Application No. 16/251,325 is a continuation of application No. 15/092,609, filed on Apr. 6, 2016, now Pat. No. 10,227,393.
(60) Provisional application No. 62/143,579, filed on Apr. 6, 2015.

(51) Int. Cl. C07K 14/71; A61K 38/45; C12N 9/12

(52) U.S. Cl. CPC C07K 14/71; A61K 38/45; C12N 9/12; C12Y 207/1103; A61K 38/00; C07K 2319/30

(57) ABSTRACT

In certain aspects, the disclosure provides soluble heteromeric polypeptide complexes comprising an extracellular domain of a type I serine/threonine kinase receptor of the TGF-beta family and an extracellular domain of a type II serine/threonine kinase receptor of the TGF-beta family. In some embodiments, the disclosure provides soluble polypeptide complexes comprising an extracellular domain of a type II receptor selected from: ActRIIA, ActRIIB, TGFBRII, BMPRII, and MISRII. In some embodiments, the disclosure provides soluble polypeptide complexes comprising an extracellular domain of a type I receptor selected from: ALK1, ALK2, ALK3, ALK4, ALK5, ALK6, and ALK7. Optionally the soluble complex is a heterodimer. In certain aspects, such soluble polypeptide complexes may be used to regulate (promote or inhibit) growth of tissues or cells including, for example, muscle, bone, cartilage, fat, neural tissue, tumors, cancerous cells, and/or cells of hematopoietic lineages, including red blood cells. In certain aspects, such soluble polypeptide complexes are can be used to improve muscle formation, bone formation, hematopoiesis, metabolic parameters, and disorders associated with these tissues, cellular networks, and endocrine systems.

### CROSS-REFERENCE TO RELATED APPLICATIONS

This application is a continuation of U.S. application Ser. No. 16/951,192, filed Nov. 18, 2020 (now abandoned), which is a continuation of U.S. application Ser. No. 16/251,325, filed Jan. 18, 2019 (now U.S. Pat. No. 10,906,958), which is a continuation of U.S. application Ser. No. 15/092,609, filed Apr. 6, 2016 (now U.S. Pat. No. 10,227,393), which claims the benefit of and priority to U.S. provisional application Ser. No. 62/143,579, filed Apr. 6, 2015 (now expired). The disclosures of each of the foregoing applications are hereby incorporated by reference in their entirety.

### SEQUENCE LISTING

The instant application contains a Sequence Listing which has been submitted electronically in XML format and is hereby incorporated by reference in its entirety. Said XML copy, created on Nov. 29, 2023, is named 1848179-0002-080-104_SL.xml and is 433,640 bytes in size.

### BACKGROUND OF THE INVENTION

The transforming growth factor-beta (TGF-beta) superfamily contains a variety of growth factors that share common sequence elements and structural motifs. These proteins are known to exert biological effects on a large variety of cell types in both vertebrates and invertebrates. Members of the superfamily perform important functions during embryonic development in pattern formation and tissue specification and can influence a variety of differentiation processes, including adipogenesis, myogenesis, chondrogenesis, cardiogenesis, hematopoiesis, neurogenesis, and epithelial cell differentiation. The family is divided into two general phylogenetic clades: the more recently evolved members of the superfamily, which includes TGF-betas, Activins, and nodal and the clade of more distantly related proteins of the superfamily, which includes a number of BMPs and GDFs. Hinck (2012) FEBS Letters 586:1860-1870. TGF-beta family members have diverse, often complementary biological effects. By manipulating the activity of a member of the TGF-beta family, it is often possible to cause significant physiological changes in an organism. For example, the Piedmontese and Belgian Blue cattle breeds carry a loss-of-function mutation in the GDF8 (also called myostatin) gene that causes a marked increase in muscle mass. Grobet et al. (1997) Nat Genet., 17(1):71-4. Furthermore, in humans, inactive alleles of GDF8 are associated with increased muscle mass and, reportedly, exceptional strength. Schuelke et al. (2004) N Engl J Med, 350:2682-8.

Changes in muscle, bone, fat, red blood cells, and other tissues may be achieved by enhancing or inhibiting signaling (e.g., SMAD 1, 2, 3, 5, and/or 8) that is mediated by ligands of the TGF-beta family. Thus, there is a need for agents that regulate the activity of various ligands of the TGF-beta superfamily.

### SUMMARY OF THE INVENTION

In part, the disclosure provides heteromultimeric complexes comprising at least one TGF-beta superfamily type I serine/threonine kinase receptor polypeptide (e.g., an ALK1, ALK2, ALK3, ALK4, ALK5, ALK6, and ALK7 polypeptide), including fragments and variants thereof, and at least one TGF-beta superfamily type II serine/threonine kinase receptor polypeptide (e.g., ActRIIA, ActRIIB, TGFBRII, BMPRII, and MISRII), including fragments and variants thereof. In other aspects, the disclosure provides heteromultimeric complexes comprising at least two different TGF-beta superfamily type I serine/threonine kinase receptor polypeptide (e.g., an ALK1, ALK2, ALK3, ALK4, ALK5, ALK6, and ALK7 polypeptide), including fragments and variants thereof. In still other aspects, the disclosure provides heteromultimeric complexes comprising at least two different TGF-beta superfamily type II serine/threonine kinase receptor polypeptide (e.g., ActRIIA, ActRIIB, TGFBRII, BMPRII, and MISRII), including fragments and variants thereof. Optionally, heteromultimeric complexes disclosed herein (e.g., an ActRIIB:ALK4 heterodimer) have different ligand binding specificities/profiles compared to their corresponding homomultimer complexes (e.g., an ActRIIB homodimer and ALK4 homodimer). Novel properties, including novel ligand binding attributes, are exhibited by heteromultimeric polypeptide complexes comprising type I and type II receptor polypeptides of the TGF-beta superfamily, as shown by Examples herein.

Heteromultimeric structures include, for example, heterodimers, heterotrimers, and higher order complexes. See, e.g., FIGS. 1, 2, 15, 16, 17, 18, 19. In some embodiments heteromultimers of the disclosure are heterodimers. Preferably, TGF-beta superfamily type I and type II receptor polypeptides as described herein comprise a ligand-binding domain of the receptor, for example, an extracellular domain of a TGF-beta superfamily type I or type II receptor. Accordingly, in certain aspects, protein complexes described herein comprise an extracellular domain of a type II TGF-beta superfamily receptor selected from: ActRIIA, ActRIIB, TGFBRII, BMPRII, and MISRII, as well as truncations and variants thereof, and an extracellular domain of a type I TGF-beta superfamily receptor selected from: ALK1, ALK2, ALK3, ALK4, ALK5, ALK6, and ALK7, as well as truncations and variants thereof. Preferably, TGF-beta superfamily type I and type II polypeptides as described herein, as well as protein complexes comprising the same, are soluble. In certain aspects, heteromultimer complexes of the disclosure bind to one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, Müllerian-inhibiting substance (MIS), and Lefty). Optionally, protein complexes of the disclosure bind to one or more of these ligands with a K_{D} of greater than or equal to 10⁻⁸, 10⁻⁹, 10⁻¹⁰, 10⁻¹¹, or 10⁻¹². In general, heteromultimers of the disclosure antagonize (inhibit) one or more activities of at least one TGF-beta superfamily ligand, and such alterations in activity may be measured using various assays known in the art, including, for example, a cell-based assay as described herein. Preferably heteromultimers of the disclosure exhibit a serum half-life of at least 4, 6, 12, 24, 36, 48, or 72 hours in a mammal (e.g., a mouse or a human). Optionally, heteromultimers of the disclosure may exhibit a serum half-life of at least 6, 8, 10, 12, 14, 20, 25, or 30 days in a mammal (e.g., a mouse or a human).

In certain aspects, heteromultimers described herein comprise a first polypeptide covalently or non-covalently associated with a second polypeptide wherein the first polypeptide comprises the amino acid sequence of a TGF-beta superfamily type I receptor polypeptide and the amino acid sequence of a first member of an interaction pair and the second polypeptide comprises the amino acid sequence of a TGF-beta superfamily type II receptor polypeptide and the amino acid sequence of a second member of the interaction pair. In other aspects, heteromultimers described herein comprise a first polypeptide covalently or non-covalently associated with a second polypeptide wherein the first polypeptide comprises the amino acid sequence of a TGF-beta superfamily type I receptor polypeptide and the amino acid sequence of a first member of an interaction pair and the second polypeptide comprises the amino acid sequence of a different TGF-beta superfamily type I receptor polypeptide and the amino acid sequence of a second member of the interaction pair. In still other aspects, heteromultimers described herein comprise a first polypeptide covalently or non-covalently associated with a second polypeptide wherein the first polypeptide comprises the amino acid sequence of a TGF-beta superfamily type II receptor polypeptide and the amino acid sequence of a first member of an interaction pair and the second polypeptide comprises the amino acid sequence of a different TGF-beta superfamily type II receptor polypeptide and the amino acid sequence of a second member of the interaction pair. Optionally, the TGF-beta superfamily type I receptor polypeptide is connected directly to the first member of the interaction pair, or an intervening sequence, such as a linker, may be positioned between the amino acid sequence of the TGF-beta superfamily type I receptor polypeptide and the amino acid sequence of the first member of the interaction pair. Similarly, the TGF-beta superfamily type II receptor polypeptide may be connected directly to the second member of the interaction pair, or an intervening sequence, such as a linker, may be positioned between the amino acid sequence of the TGF-beta superfamily type II receptor polypeptide and the amino acid sequence of the second member of the interaction pair. Examples of linkers include, but are not limited to, the sequences TGGG (SEQ ID NO: 62), TGGGG (SEQ ID NO: 60), SGGGG (SEQ ID NO: 61), GGGG (SEQ ID NO: 59), and GGG (SEQ ID NO: 58).

Interaction pairs described herein are designed to promote dimerization or form higher order multimers. In some embodiments, the interaction pair may be any two polypeptide sequences that interact to form a complex, particularly a heterodimeric complex although operative embodiments may also employ an interaction pair that forms a homodimeric sequence. The first and second members of the interaction pair may be an asymmetric pair, meaning that the members of the pair preferentially associate with each other rather than self-associate. Accordingly, first and second members of an asymmetric interaction pair may associate to form a heterodimeric complex. Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer complex or a heterodimeric complex. Optionally, the first member of the interaction action pair (e.g., an asymmetric pair or an unguided interaction pair) associates covalently with the second member of the interaction pair. Optionally, the first member of the interaction action pair (e.g., an asymmetric pair or an unguided interaction pair) associates non-covalently with the second member of the interaction pair. Optionally, the first member of the interaction pair (e.g., an asymmetrical or an unguided interaction pair) associates through both covalent and non-covalent mechanisms with the second member of the interaction pair.

In some embodiments, TGF-beta superfamily type I receptor polypeptides are fusion proteins that comprise an Fc domain of an immunoglobulin. Similarly, in some embodiments, TGF-beta superfamily type II receptor polypeptides are fusion proteins that comprise an Fc domain of an immunoglobulin. Traditional Fc fusion proteins and antibodies are examples of unguided interaction pairs, whereas a variety of engineered Fc domains have been designed as asymmetric interaction pairs [Spiess et al (2015) Molecular Immunology 67(2A): 95-106]. Therefore, a first member and/or a second member of an interaction pair described herein may comprise a constant domain of an immunoglobulin, including, for example, the Fc portion of an immunoglobulin. For example, a first member of an interaction pair may comprise an amino acid sequence that is derived from an Fc domain of an IgG (IgG1, IgG2, IgG3, or IgG4), IgA (IgA1 or IgA2), IgE, or IgM immunoglobulin. For example, the first member of an interaction pair may comprise, consist essentially of, or consist of an amino acid sequence that is at least 80%, 85%, 90%, 91%, 92%, 93%, 94%, 95%, 96%, 97%, 98%, 99% or 100% identical to any one of SEQ ID NOs: 200-214. Optionally, a second member of an interaction pair may comprise an amino acid sequence that is derived from an Fc domain of an IgG (IgG1, IgG2, IgG3, or IgG4), IgA (IgA1 or IgA2), IgE, or IgM. Such immunoglobulin domains may comprise one or more amino acid modifications (e.g., deletions, additions, and/or substitutions) that promote heterodimer formation. For example, the second member of an interaction pair may comprise, consist essentially of, or consist of an amino acid sequence that is at least 80%, 85%, 90%, 91%, 92%, 93%, 94%, 95%, 96%, 97%, 98%, 99% or 100% identical to any one of SEQ ID NOs: 200-214. In some embodiments, a first member and a second member of an interaction pair comprise Fc domains derived from the same immunoglobulin class and subtype. In other embodiments, a first member and a second member of an interaction pair comprise Fc domains derived from different immunoglobulin classes or subtypes. Similarly, a first member and/or a second member of an interaction pair (e.g., an asymmetric pair or an unguided interaction pair) comprise a modified constant domain of an immunoglobulin, including, for example, a modified Fc portion of an immunoglobulin. For example, protein complexes of the disclosure may comprise a first modified Fc portion of an immunoglobulin comprising an amino acid sequence that is at least 80%, 85%, 90%, 91%, 92%, 93%, 94%, 95%, 96%, 97%, 98%, or 99% identical to an amino acid sequence selected from the group: SEQ ID NOs: 200-214 and a second modified Fc portion of an immunoglobulin, which may be the same or different from the amino acid sequence of the first modified Fc portion of the immunoglobulin, comprising an amino acid sequence that is at least 80%, 85%, 90%, 91%, 92%, 93%, 94%, 95%, 96%, 97%, 98%, or 99% identical to an amino acid sequence selected from the group: SEQ ID NOs: 200-214. Such immunoglobulin domains may comprise one or more amino acid modifications (e.g., deletions, additions, and/or substitutions) that promote heterodimer formation.

In some embodiments, the disclosure provides heteromeric polypeptide complexes comprising a type I and type II TGF-beta superfamily receptor polypeptide, wherein the type II TGF-beta superfamily receptor polypeptide is derived from an ActRIIA receptor. In some embodiments, the disclosure provides heteromeric polypeptide complexes comprising at least two different type II TGF-beta superfamily receptor polypeptide, wherein at least one of the type II TGF-beta superfamily receptor polypeptide is derived from an ActRIIA receptor. For example, ActRIIA polypeptides may comprise, consist essentially of, or consist of an amino acid sequence that is at least 70%, 80%, 85%, 90%, 95%, 97%, 98%, 99% or 100% identical to an ActRIIA sequence disclosed herein (e.g., SEQ ID NOs: 9, 10, 11, 118, 120, 151, 152, 409, 410, 451, and 452). Optionally, ActRIIA polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 21-30 (e.g., amino acid residues 21, 22, 23, 24, 25, 26, 27, 28, 29, or 30) SEQ ID NO: 9, and b) ends at any one of amino acids 110-135 (e.g., 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 9. Optionally, ActRIIA polypeptides of the disclosure may be fusion proteins that further comprise one or more portions (domains) that are heterologous to ActRIIA. For example, an ActRIIA polypeptide may be fused to a heterologous polypeptide that comprises a multimerization domain, optionally with a linker domain positioned between the ActRIIA polypeptide and the heterologous polypeptide. In some embodiments, multimerization domains described herein comprise one component of an interaction pair. In certain aspects, heteromeric complexes that comprise an ActRIIA polypeptide further comprise at least one type I TGF-beta superfamily receptor polypeptide. For example, an ActRIIA heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. Optionally, ALK1 polypeptides in this and other embodiments may comprise, consist essentially of, or consist of an amino acid sequence that is at least 70%, 80%, 85%, 90%, 95%, 97%, 98%, 99% or 100% identical to a polypeptide that a) begins at any one of amino acids of 22-34 (e.g., amino acid residues 22, 23, 24, 25, 26, 27, 28, 29, 30, 31, 32, 33, and 34) SEQ ID NO: 14, and b) ends at any one of amino acids 95-118 (e.g., amino acid residues 95, 96, 97, 98, 99, 100, 101, 102, 103, 104, 105, 106, 107, 108, 109, 110, 111, 112, 113, 114, 115, 116, 117, and 118) of SEQ ID NO: 14. In some embodiments, an ActRIIA heteromeric complex may further comprise an ALK2 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. Optionally, ALK2 polypeptides in this and other embodiments may comprise, consist essentially of, or consist of an amino acid sequence that is at least 70%, 80%, 85%, 90%, 95%, 97%, 98%, 99% or 100% identical to a polypeptide that a) begins at any one of amino acids of 21-35 (e.g., amino acid residues 21, 22, 23, 24, 25, 26, 27, 28, 29, 30, 31, 32, 33, 34, and 35) SEQ ID NO: 18, and b) ends at any one of amino acids 99-123 (e.g., amino acid residues 99, 100, 101, 102, 103, 104, 105, 106, 107, 108, 109, 110, 111, 112, 113, 114, 115, 116, 117, 118, 119, 120, 121, 122, and 123) of SEQ ID NO: 18. In some embodiments, an ActRIIA heteromeric complex may further comprise an ALK3 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. Optionally, in this and other embodiments ALK3 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 24-61 (e.g., amino acid residues 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, and 61) SEQ ID NO: 22, and b) ends at any one of amino acids 130-152 (e.g., amino acid residues 130, 131, 132, 133, 134, 135, 136, 137, 138, 139, 140, 141, 142, 143, 144, 145, 146, 147, 148, 149, 150, 151, and 152) of SEQ ID NO: 22. In some embodiments, an ActRIIA heteromeric complex may further comprise an ALK4 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. Optionally, in this and other embodiments ALK4 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 23-34 (e.g., amino acid residues 23, 24, 25, 26, 27, 28, 29, 30, 31, 32, 33, 34) SEQ ID NO: 26 or 83, and b) ends at any one of amino acids 101-126 (e.g., amino acid residues 101, 102, 103, 104, 105, 106, 107, 108, 109, 110, 111, 112, 113, 114, 115, 116, 117, 118, 119, 120, 121, 122, 123, 124, 125, and 126) of SEQ ID NO: 26 or 83. In some embodiments, an ActRIIA heteromeric complex may further comprise an ALK5 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. Optionally, in this and other embodiments ALK5 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 25-36 (e.g., amino acid residues 25, 26, 27, 28, 29, 30, 31, 32, 33, 34, 35, and 36) SEQ ID NO: 30 or 87, and b) ends at any one of amino acids 106-126 (e.g., amino acid residues 106, 107, 108, 109, 110, 111, 112, 113, 114, 115, 116, 117, 118, 119, 120, 121, 122, 123, 124, 125, and 126) of SEQ ID NO: 30 or 87. In some embodiments, an ActRIIA heteromeric complex may further comprise an ALK6 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. Optionally, in this and other embodiments ALK6 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 14-32 (e.g., amino acid residues 14, 15, 16, 17, 18, 19, 20, 21, 22, 23, 24, 25, 26, 27, 28, 29, 30, 31, and 32) SEQ ID NO: 34, and b) ends at any one of amino acids 102-126 (e.g., amino acid residues 102, 103, 104, 105, 106, 107, 108, 109, 110, 111, 112, 113, 114, 115, 116, 117, 118, 119, 120, 121, 122, 123, 124, 125, and 126) of SEQ ID NO: 34. Optionally, in this and other embodiments ALK6 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 26-62 (e.g., amino acid residues 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, and 62) SEQ ID NO: 91, and b) ends at any one of amino acids 132-156 (e.g., amino acid residues 132, 133, 134, 135, 136, 137, 138, 139, 140, 141, 142, 143, 144, 145, 146, 147, 148, 149, 150, 151, 152, 153, 154, 155, and 156) of SEQ ID NO: 91. In some embodiments, an ActRIIA heteromeric complex may further comprise an ALK7 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476. Optionally, in this and other embodiments, ALK7 polypeptides may comprise, consist essentially of, or consist of an amino acid sequence that is at least 70%, 80%, 85%, 90%, 95%, 97%, 98%, 99% or 100% identical to a polypeptide that begins at any one of amino acids 21-28 of SEQ ID NO: 38 (e.g., amino acids 21, 22, 23, 24, 25, 26, 27, or 28) and ends at any one of amino acids 92-113 of SEQ ID NO: 38 (e.g., amino acids 92, 93, 94, 95, 96, 97, 98, 99, 100, 101, 102, 103, 104, 105, 106, 107, 108, 109, 110, 111, 112, or 113 of SEQ ID NO: 38). In certain aspects, heteromeric complexes that comprise an ActRIIA polypeptide further comprise at least one different type II TGF-beta superfamily receptor polypeptide. For example, an ActRIIA heteromeric complex may further comprise an ActRIIB polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 1, 2, 3, 4, 5, 6, 100, 102, 153, 154, 401, 402, 453, and 454. In some embodiments, an ActRIIA heteromeric complex may further comprise an BMPRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 46, 47, 71, 72, 121, 123, 155, 156, 411, 412, 455, and 456. In some embodiments, an ActRIIA heteromeric complex may further comprise an MISRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 50, 51, 75, 76, 79, 80, 133, 135, 161, 162, 419, 420, 457, and 458. In some embodiments, an ActRIIA heteromeric complex may further comprise an TGFBRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 42, 43, 67, 68, 127, 129, 130, 132, 157, 158, 159, 160, 415, 416, 417, 418, 459, 460, 461, and 462.

In some embodiments, the disclosure provides heteromeric polypeptide complexes comprising a type I and type II TGF-beta superfamily receptor polypeptide, wherein the type II TGF-beta superfamily receptor polypeptide is derived from an ActRIIB receptor. In some embodiments, the disclosure provides heteromeric polypeptide complexes comprising at least two different type II TGF-beta superfamily receptor polypeptide, wherein at least one of the type II TGF-beta superfamily receptor polypeptide is derived from an ActRIIB receptor. For example, ActRIIB polypeptides may comprise, consist essentially of, or consist of an amino acid sequence that is at least 70%, 80%, 85%, 90%, 95%, 97%, 98%, 99% or 100% identical to an ActRIIB sequence disclosed herein (e.g., SEQ ID NOs: 1, 2, 3, 4, 5, 6, 100, 102, 153, 154, 401, 402, 453, and 454). Optionally, ActRIIB polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 20-29 (e.g., amino acid residues 20, 21, 22, 23, 24, 25, 26, 27, 28, or 29) SEQ ID NO: 1, and b) ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 1. Optionally, ActRIIB polypeptides of the disclosure may be fusion proteins that further comprise one or more portions (domains) that are heterologous to ActRIIB. For example, an ActRIIB polypeptide may be fused to a heterologous polypeptide that comprises a multimerization domain, optionally with a linker domain positioned between the ActRIIB polypeptide and the heterologous polypeptide. In some embodiments, multimerization domains described herein comprise one component of an interaction pair. Preferably, heteromeric complexes that comprise an ActRIIB polypeptide further comprise at least one type I TGF-beta superfamily receptor polypeptide. For example, an ActRIIB heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. In some embodiments, an ActRIIB heteromeric complex may further comprise an ALK2 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, an ActRIIB heteromeric complex may further comprise an ALK3 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, an ActRIIB heteromeric complex may further comprise an ALK4 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, an ActRIIB heteromeric complex may further comprise an ALK5 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, an ActRIIB heteromeric complex may further comprise an ALK6 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, an ActRIIB heteromeric complex may further comprise an ALK7 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476. In certain aspects, heteromeric complexes that comprise an ActRIIB polypeptide further comprise at least one different type II TGF-beta superfamily receptor polypeptide. For example, an ActRIIA heteromeric complex may further comprise an ActRIIB polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 1, 2, 3, 4, 5, 6, 100, 102, 153, 154, 401, 402, 453, and 454. For example, an ActRIIB heteromeric complex may further comprise an ActRIIA polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 9, 10, 11, 118, 120, 151, 152, 409, 410, 451, and 452. In some embodiments, an ActRIIB heteromeric complex may further comprise an BMPRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 46, 47, 71, 72, 121, 123, 155, 156, 411, 412, 455, and 456. In some embodiments, an ActRIIB heteromeric complex may further comprise an MISRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 50, 51, 75, 76, 79, 80, 133, 135, 161, 162, 419, 420, 457, and 458. In some embodiments, an ActRIIB heteromeric complex may further comprise an TGFBRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 42, 43, 67, 68, 127, 129, 130, 132, 157, 158, 159, 160, 415, 416, 417, 418, 459, 460, 461, and 462.

In some embodiments, the disclosure provides heteromeric polypeptide complexes comprising a type I and type II TGF-beta superfamily receptor polypeptide, wherein the type II TGF-beta superfamily receptor polypeptide is derived from a TGFBRII receptor. In some embodiments, the disclosure provides heteromeric polypeptide complexes comprising at least two different type II TGF-beta superfamily receptor polypeptide, wherein at least one of the type II TGF-beta superfamily receptor polypeptide is derived from an TGFBRII receptor. For example, TGFBRII polypeptides may comprise, consist essentially of, or consist of an amino acid sequence that is at least 70%, 80%, 85%, 90%, 95%, 97%, 98%, 99% or 100% identical to an TGFBRII sequence disclosed herein (e.g., SEQ ID NOs: 42, 43, 67, 68, 127, 129, 130, 132, 157, 158, 159, 160, 415, 416, 417, 418, 459, 460, 461, and 462). Optionally, TGFBRII polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 23, 24, 25, 26, 27, 28, 29, 30, 31, 32, 33, 34, 35, 36, 37, 38, 39, 40, 41, 42, 43, 44, 45, 46, 47, 48, 49, 50 or 51 of SEQ ID NO: 42, and b) ends at any one of amino acids 143, 144, 145, 146, 147, 148, 149, 150, 151, 152, 153, 154, 155, 156, 157, 158, 159, 160, 161, 162, 163, 164, 165 or 166 of SEQ ID NO: 42. Optionally, TGFBRII polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 23, 24, 25, 26, 27, 28, 29, 30, 31, 32, 33, 34, 35, 36, 37, 38, 39, 40, 41, 42, 43 or 44 of SEQ ID NO: 67, and b) ends at any one of amino acids 163, 164, 165, 166, 167, 168, 169, 170, 171, 172, 173, 174, 175, 176, 177, 178, 179, 180, 181, 182, 183, 184, 185, 186, 187, 188, 189, 190 or 191 of SEQ ID NO: 67. Optionally, TGFBRII polypeptides of the disclosure may be fusion proteins that further comprise one or more portions (domains) that are heterologous to TGFBRII. For example, a TGFBRII polypeptide may be fused to a heterologous polypeptide that comprises a multimerization domain, optionally with a linker domain positioned between the TGFBRII polypeptide and the heterologous polypeptide. In some embodiments, multimerization domains described herein comprise one component of an interaction pair. Preferably, heteromeric complexes that comprise a TGFBRII polypeptide further comprise at least one type I TGF-beta superfamily receptor polypeptide. For example, a TGFBRII heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. In some embodiments, a TGFBRII heteromeric complex may further comprise an ALK2 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, a TGFBRII heteromeric complex may further comprise an ALK3 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, a TGFBRII heteromeric complex may further comprise an ALK4 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, a TGFBRII heteromeric complex may further comprise an ALK5 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, a TGFBRII heteromeric complex may further comprise an ALK6 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, a TGFBRII heteromeric complex may further comprise an ALK7 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476. Optionally, heteromeric complexes comprising a TGFBRII polypeptide may further comprise one or more additional type II TGF-beta superfamily receptor polypeptides, including, for example ActRIIA, ActRIIB, BMPRII, and MISRII polypeptides described herein (e.g., a polypeptide comprising, consisting essentially of, or consisting of an amino acid sequence selected from SEQ ID NOs: 1, 2, 3, 4, 5, 6, 9, 10, 11 46, 47, 50, 51, 71, 72, 75, 76, 79, 80, 100, 102, 118, 120, 121, 123, 401, 402, 409, 410, 411, and 412). In certain aspects, heteromeric complexes that comprise an TGFBRII polypeptide further comprise at least one different type II TGF-beta superfamily receptor polypeptide. For example, an TGFBRII heteromeric complex may further comprise an ActRIIA polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 9, 10, 11, 118, 120, 151, 152, 409, 410, 451, and 452. In some embodiments, a TGFBRII heteromeric complex may further comprise an ActRIIB polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 1, 2, 3, 4, 5, 6, 100, 102, 153, 154, 401, 402, 453, and 454. In some embodiments, a TGFBRII heteromeric complex may further comprise an MISRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 50, 51, 75, 76, 79, 80, 133, 135, 161, 162, 419, 420, 457, and 458. In some embodiments, a TGFBRII heteromeric complex may further comprise an BMPRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 46, 47, 71, 72, 121, 123, 155, 156, 411, 412, 455, and 456.

In some embodiments, the disclosure provides heteromeric polypeptide complexes comprising a type I and type II TGF-beta superfamily receptor polypeptide, wherein the type II TGF-beta superfamily receptor polypeptide is derived from a BMPRII receptor. In some embodiments, the disclosure provides heteromeric polypeptide complexes comprising at least two different type II TGF-beta superfamily receptor polypeptide, wherein at least one of the type II TGF-beta superfamily receptor polypeptide is derived from an BMPRII receptor. For example, BMPRII polypeptides may comprise, consist essentially of, or consist of an amino acid sequence that is at least 70%, 80%, 85%, 90%, 95%, 97%, 98%, 99% or 100% identical to a BMPRII sequence disclosed herein (e.g., SEQ ID NOs: 46, 47, 71, 72, 121, 123, 155, 156, 411, 412, 455, and 456). Optionally, BMPRII polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 27-34 (e.g., amino acid residues 27, 28, 29, 30, 31, 32, 33, and 34) SEQ ID NO: 46 or 71, and b) ends at any one of amino acids 123-150 (e.g., amino acid residues 123, 124, 125, 126, 127, 128, 129, 130, 131, 132, 133, 134, 135, 136, 137, 138, 139, 140, 141, 142, 143, 144, 145, 146, 147, 148, 149, and 150) of SEQ ID NO: 46 or 71. Optionally, BMPRII polypeptides of the disclosure may be fusion proteins that further comprise one or more portions (domains) that are heterologous to BMPRII. For example, a BMPRII polypeptide may be fused to a heterologous polypeptide that comprises a multimerization domain, optionally with a linker domain positioned between the BMPRII polypeptide and the heterologous polypeptide. In some embodiments, multimerization domains described herein comprise one component of an interaction pair. Preferably, heteromeric complexes that comprise a BMPRII polypeptide further comprise at least one type I TGF-beta superfamily receptor polypeptide. For example, a BMPRII heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. In some embodiments, a BMPRII heteromeric complex may further comprise an ALK2 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, a BMPRII heteromeric complex may further comprise an ALK3 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, a BMPRII heteromeric complex may further comprise an ALK4 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, a BMPRII heteromeric complex may further comprise an ALK5 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, a BMPRII heteromeric complex may further comprise an ALK6 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, a BMPRII heteromeric complex may further comprise an ALK7 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476. In certain aspects, heteromeric complexes that comprise an BMPRII polypeptide further comprise at least one different type II TGF-beta superfamily receptor polypeptide. For example, an BMPRII heteromeric complex may further comprise an ActRIIA polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 9, 10, 11, 118, 120, 151, 152, 409, 410, 451, and 452. In some embodiments, a BMPRII heteromeric complex may further comprise an ActRIIB polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 1, 2, 3, 4, 5, 6, 100, 102, 153, 154, 401, 402, 453, and 454. In some embodiments, a BMPRII heteromeric complex may further comprise an MISRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 50, 51, 75, 76, 79, 80, 133, 135, 161, 162, 419, 420, 457, and 458. In some embodiments, a BMPRII heteromeric complex may further comprise an TGFBRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 42, 43, 67, 68, 127, 129, 130, 132, 157, 158, 159, 160, 415, 416, 417, 418, 459, 460, 461, and 462.

In some embodiments, the disclosure provides heteromeric polypeptide complexes comprising a type I and type II TGF-beta superfamily receptor polypeptide, wherein the type II TGF-beta superfamily receptor polypeptide is derived from an MISRII receptor. In some embodiments, the disclosure provides heteromeric polypeptide complexes comprising at least two different type II TGF-beta superfamily receptor polypeptide, wherein at least one of the type II TGF-beta superfamily receptor polypeptide is derived from an MISRII receptor. In some embodiments, the disclosure provides heteromeric polypeptide complexes comprising at least two different type II TGF-beta superfamily receptor polypeptide, wherein at least one of the type II TGF-beta superfamily receptor polypeptide is derived from an MISRII receptor. For example, MISRII polypeptides may comprise, consist essentially of, or consist of an amino acid sequence that is at least 70%, 80%, 85%, 90%, 95%, 97%, 98%, 99% or 100% identical to an MISRII sequence disclosed herein (e.g., SEQ ID NOs: 50, 51, 75, 76, 79, 80, 133, 135, 161, 162, 419, 420, 457, and 458). Optionally, MISRII polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 17-24 (e.g., amino acid residues 17, 18, 19, 20, 21, 22, 23, and 24) SEQ ID NO: 50, 75, or 79, and b) ends at any one of amino acids 116-149 (e.g., amino acid residues 116, 117, 118, 119, 120, 121, 122 123, 124, 125, 126, 127, 128, 129, 130, 131, 132, 133, 134, 135, 136, 137, 138, 139, 140, 141, 142, 143, 144, 145, 146, 147, 148, and 149) of SEQ ID NO: 50, 75, or 79. Optionally, MISRII polypeptides of the disclosure may be fusion proteins that further comprise one or more portions (domains) that are heterologous to MISRII. For example, an MISRII polypeptide may be fused to a heterologous polypeptide that comprises a multimerization domain, optionally with a linker domain positioned between the MISRII polypeptide and the heterologous polypeptide. In some embodiments, multimerization domains described herein comprise one component of an interaction pair. Preferably, heteromeric complexes that comprise an MISRII polypeptide further comprise at least one type I TGF-beta superfamily polypeptide. For example, an MISRII heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. In some embodiments, an MISRII heteromeric complex may further comprise an ALK2 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, an MISRII heteromeric complex may further comprise an ALK3 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, an MISRII heteromeric complex may further comprise an ALK4 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, an MISRII heteromeric complex may further comprise an ALK5 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, an MISRII heteromeric complex may further comprise an ALK6 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, an MISRII heteromeric complex may further comprise an ALK7 polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476. In certain aspects, heteromeric complexes that comprise an MISRII polypeptide further comprise at least one different type II TGF-beta superfamily receptor polypeptide. For example, an MISRII heteromeric complex may further comprise an ActRIIA polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 9, 10, 11, 118, 120, 151, 152, 409, 410, 451, and 452. In some embodiments, an MISRII heteromeric complex may further comprise an ActRIIB polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 1, 2, 3, 4, 5, 6, 100, 102, 153, 154, 401, 402, 453, and 454. In some embodiments, an MISRII heteromeric complex may further comprise an BMPRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 46, 47, 71, 72, 121, 123, 155, 156, 411, 412, 455, and 456. In some embodiments, an MISRII heteromeric complex may further comprise an TGFBRII polypeptide as described herein, including, for example, a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 42, 43, 67, 68, 127, 129, 130, 132, 157, 158, 159, 160, 415, 416, 417, 418, 459, 460, 461, and 462.

In some embodiments, the disclosure provides heteromeric polypeptide complexes comprising at least two different type I TGF-beta superfamily receptor polypeptide, wherein at least one of the type I TGF-beta superfamily receptor polypeptide is derived from an ALK1 receptor. For example, ALK1 polypeptides may comprise, consist essentially of, or consist of an amino acid sequence that is at least 70%, 80%, 85%, 90%, 95%, 97%, 98%, 99% or 100% identical to an ALK1 sequence disclosed herein (e.g., 14, 15, 124, 126, 171, 172, 413, 414, 463, and 464). Optionally, ALK1 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 22-34 (e.g., amino acid residues 22, 23, 24, 25, 26, 27, 28, 29, 30, 31, 32, 33, and 34) SEQ ID NO: 14, and b) ends at any one of amino acids 95-118 (e.g., amino acid residues 95, 96, 97, 98, 99, 100, 101, 102, 103, 104, 105, 106, 107, 108, 109, 110, 111, 112, 113, 114, 115, 116, 117, and 118) of SEQ ID NO: 14. Optionally, ALK1 polypeptides of the disclosure may be fusion proteins that further comprise one or more portions (domains) that are heterologous to ALK1. For example, an ALK1 polypeptide may be fused to a heterologous polypeptide that comprises a multimerization domain, optionally with a linker domain positioned between the ALK1 polypeptide and the heterologous polypeptide. In some embodiments, multimerization domains described herein comprise one component of an interaction pair. In some embodiments, heteromeric complexes that comprise an ALK1 polypeptide further comprise at least one different type I TGF-beta superfamily polypeptide. For example, an ALK1 heteromeric complex may further comprise an ALK2 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, an ALK1 heteromeric complex may further comprise an ALK3 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, an ALK1 heteromeric complex may further comprise an ALK4 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, an ALK1 heteromeric complex may further comprise an ALK5 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, an ALK1 heteromeric complex may further comprise an ALK6 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, an ALK1 heteromeric complex may further comprise an ALK7 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476.

In some embodiments, the disclosure provides heteromeric polypeptide complexes comprising at least two different type I TGF-beta superfamily receptor polypeptide, wherein at least one of the type I TGF-beta superfamily receptor polypeptide is derived from an ALK2 receptor. For example, ALK2 polypeptides may comprise, consist essentially of, or consist of an amino acid sequence that is at least 70%, 80%, 85%, 90%, 95%, 97%, 98%, 99% or 100% identical to an ALK2 sequence disclosed herein (e.g., 18, 19, 136, 138, 173, 174, 421, 422, 465, and 466). Optionally, ALK2 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 21-35 (e.g., amino acid residues 21, 22, 23, 24, 25, 26, 27, 28, 29, 30, 31, 32, 33, 34, and 35) SEQ ID NO: 18, and b) ends at any one of amino acids 99-123 (e.g., amino acid residues 99, 100, 101, 102, 103, 104, 105, 106, 107, 108, 109, 110, 111, 112, 113, 114, 115, 116, 117, 118, 119, 120, 121, 122, and 123) of SEQ ID NO: 18. Optionally, ALK2 polypeptides of the disclosure may be fusion proteins that further comprise one or more portions (domains) that are heterologous to ALK2. For example, an ALK2 polypeptide may be fused to a heterologous polypeptide that comprises a multimerization domain, optionally with a linker domain positioned between the ALK2 polypeptide and the heterologous polypeptide. In some embodiments, multimerization domains described herein comprise one component of an interaction pair. In some embodiments, heteromeric complexes that comprise an ALK2 polypeptide further comprise at least one different type I TGF-beta superfamily polypeptide. For example, an ALK2 heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. In some embodiments, an ALK2 heteromeric complex may further comprise an ALK3 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, an ALK2 heteromeric complex may further comprise an ALK4 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, an ALK2 heteromeric complex may further comprise an ALK5 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, an ALK2 heteromeric complex may further comprise an ALK6 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, an ALK2 heteromeric complex may further comprise an ALK7 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476.

In some embodiments, the disclosure provides heteromeric polypeptide complexes comprising at least two different type I TGF-beta superfamily receptor polypeptide, wherein at least one of the type I TGF-beta superfamily receptor polypeptide is derived from an ALK3 receptor. For example, ALK3 polypeptides may comprise, consist essentially of, or consist of an amino acid sequence that is at least 70%, 80%, 85%, 90%, 95%, 97%, 98%, 99% or 100% identical to an ALK3 sequence disclosed herein (e.g., 22, 23, 115, 117, 175, 176, 407, 408, 467, and 468). Optionally, ALK3 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 24-61 (e.g., amino acid residues 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, and 61) SEQ ID NO: 22, and b) ends at any one of amino acids 130-152 (e.g., amino acid residues 130, 131, 132, 133, 134, 135, 136, 137, 138, 139, 140, 141, 142, 143, 144, 145, 146, 147, 148, 149, 150, 151, and 152) of SEQ ID NO: 22. Optionally, ALK3 polypeptides of the disclosure may be fusion proteins that further comprise one or more portions (domains) that are heterologous to ALK3. For example, an ALK3 polypeptide may be fused to a heterologous polypeptide that comprises a multimerization domain, optionally with a linker domain positioned between the ALK3 polypeptide and the heterologous polypeptide. In some embodiments, multimerization domains described herein comprise one component of an interaction pair. In some embodiments, heteromeric complexes that comprise an ALK3 polypeptide further comprise at least one different type I TGF-beta superfamily polypeptide. For example, an ALK3 heteromeric complex may further comprise an ALK2 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, an ALK3 heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. In some embodiments, an ALK3 heteromeric complex may further comprise an ALK4 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, an ALK3 heteromeric complex may further comprise an ALK5 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, an ALK3 heteromeric complex may further comprise an ALK6 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, an ALK3 heteromeric complex may further comprise an ALK7 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476.

In some embodiments, the disclosure provides heteromeric polypeptide complexes comprising at least two different type I TGF-beta superfamily receptor polypeptide, wherein at least one of the type I TGF-beta superfamily receptor polypeptide is derived from an ALK4 receptor. For example, ALK4 polypeptides may comprise, consist essentially of, or consist of an amino acid sequence that is at least 70%, 80%, 85%, 90%, 95%, 97%, 98%, 99% or 100% identical to an ALK4 sequence disclosed herein (e.g., 26, 27, 83, 84, 104, 106, 177, 178, 403, 404, 469, and 470). Optionally, ALK4 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 23-34 (e.g., amino acid residues 23, 24, 25, 26, 27, 28, 29, 30, 31, 32, 33, 34) SEQ ID NO: 26 or 83, and b) ends at any one of amino acids 101-126 (e.g., amino acid residues 101, 102, 103, 104, 105, 106, 107, 108, 109, 110, 111, 112, 113, 114, 115, 116, 117, 118, 119, 120, 121, 122, 123, 124, 125, and 126) of SEQ ID NO: 26 or 83. Optionally, ALK4 polypeptides of the disclosure may be fusion proteins that further comprise one or more portions (domains) that are heterologous to ALK4. For example, an ALK4 polypeptide may be fused to a heterologous polypeptide that comprises a multimerization domain, optionally with a linker domain positioned between the ALK4 polypeptide and the heterologous polypeptide. In some embodiments, multimerization domains described herein comprise one component of an interaction pair. In some embodiments, heteromeric complexes that comprise an ALK4 polypeptide further comprise at least one different type I TGF-beta superfamily polypeptide. For example, an ALK4 heteromeric complex may further comprise an ALK2 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, an ALK4 heteromeric complex may further comprise an ALK3 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, an ALK4 heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. In some embodiments, an ALK4 heteromeric complex may further comprise an ALK5 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, an ALK4 heteromeric complex may further comprise an ALK6 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, an ALK4 heteromeric complex may further comprise an ALK7 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476.

In some embodiments, the disclosure provides heteromeric polypeptide complexes comprising at least two different type I TGF-beta superfamily receptor polypeptide, wherein at least one of the type I TGF-beta superfamily receptor polypeptide is derived from an ALK5 receptor. For example, ALK5 polypeptides may comprise, consist essentially of, or consist of an amino acid sequence that is at least 70%, 80%, 85%, 90%, 95%, 97%, 98%, 99% or 100% identical to an ALK5 sequence disclosed herein (e.g., 30, 31, 87, 88, 139, 141, 179, 180, 423, 424, 471, and 472). Optionally, ALK5 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 25-36 (e.g., amino acid residues 25, 26, 27, 28, 29, 30, 31, 32, 33, 34, 35, and 36) SEQ ID NO: 30 or 87, and b) ends at any one of amino acids 106-126 (e.g., amino acid residues 106, 107, 108, 109, 110, 111, 112, 113, 114, 115, 116, 117, 118, 119, 120, 121, 122, 123, 124, 125, and 126) of SEQ ID NO: 30 or 87. Optionally, ALK5 polypeptides of the disclosure may be fusion proteins that further comprise one or more portions (domains) that are heterologous to ALK5. For example, an ALK5 polypeptide may be fused to a heterologous polypeptide that comprises a multimerization domain, optionally with a linker domain positioned between the ALK5 polypeptide and the heterologous polypeptide. In some embodiments, multimerization domains described herein comprise one component of an interaction pair. In some embodiments, heteromeric complexes that comprise an ALK5 polypeptide further comprise at least one different type I TGF-beta superfamily polypeptide. For example, an ALK5 heteromeric complex may further comprise an ALK2 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, an ALK5 heteromeric complex may further comprise an ALK3 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, an ALK5 heteromeric complex may further comprise an ALK4 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, an ALK5 heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. In some embodiments, an ALK5 heteromeric complex may further comprise an ALK6 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, an ALK5 heteromeric complex may further comprise an ALK7 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476.

In some embodiments, the disclosure provides heteromeric polypeptide complexes comprising at least two different type I TGF-beta superfamily receptor polypeptide, wherein at least one of the type I TGF-beta superfamily receptor polypeptide is derived from an ALK6 receptor. For example, ALK6 polypeptides may comprise, consist essentially of, or consist of an amino acid sequence that is at least 70%, 80%, 85%, 90%, 95%, 97%, 98%, 99% or 100% identical to an ALK6 sequence disclosed herein (e.g., 34, 35, 91, 92, 142, 144, 181, 182, 425, 426, 473, and 474). Optionally, ALK6 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 14-32 (e.g., amino acid residues 14, 15, 16, 17, 18, 19, 20, 21, 22, 23, 24, 25, 26, 27, 28, 29, 30, 31, and 32) SEQ ID NO: 34, and b) ends at any one of amino acids 102-126 (e.g., amino acid residues 102, 103, 104, 105, 106, 107, 108, 109, 110, 111, 112, 113, 114, 115, 116, 117, 118, 119, 120, 121, 122, 123, 124, 125, and 126) of SEQ ID NO: 34. Optionally, ALK6 polypeptides may comprise, consist essentially of, or consist of an amino acid sequence that is at least 70%, 80%, 85%, 90%, 95%, 97%, 98%, 99% or 100% identical to a polypeptide that a) begins at any one of amino acids of 26-62 (e.g., amino acid residues 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, and 62) SEQ ID NO: 91, and b) ends at any one of amino acids 132-156 (e.g., amino acid residues 132, 133, 134, 135, 136, 137, 138, 139, 140, 141, 142, 143, 144, 145, 146, 147, 148, 149, 150, 151, 152, 153, 154, 155, and 156) of SEQ ID NO: 91. Optionally, ALK6 polypeptides of the disclosure may be fusion proteins that further comprise one or more portions (domains) that are heterologous to ALK6. For example, an ALK6 polypeptide may be fused to a heterologous polypeptide that comprises a multimerization domain, optionally with a linker domain positioned between the ALK6 polypeptide and the heterologous polypeptide. In some embodiments, multimerization domains described herein comprise one component of an interaction pair. In some embodiments, heteromeric complexes that comprise an ALK6 polypeptide further comprise at least one different type I TGF-beta superfamily polypeptide. For example, an ALK6 heteromeric complex may further comprise an ALK2 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, an ALK6 heteromeric complex may further comprise an ALK3 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, an ALK6 heteromeric complex may further comprise an ALK4 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, an ALK6 heteromeric complex may further comprise an ALK5 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, an ALK6 heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464. In some embodiments, an ALK6 heteromeric complex may further comprise an ALK7 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 38, 39, 301, 302, 305, 306, 309, 310, 313, 112, 114, 183, 184, 405, 406, 475, and 476.

In some embodiments, the disclosure provides heteromeric polypeptide complexes comprising at least two different type I TGF-beta superfamily receptor polypeptide, wherein at least one of the type I TGF-beta superfamily receptor polypeptide is derived from an ALK7 receptor. For example, ALK7 polypeptides may comprise, consist essentially of, or consist of an amino acid sequence that is at least 70%, 80%, 85%, 90%, 95%, 97%, 98%, 99% or 100% identical to an ALK7 sequence disclosed herein (e.g., 38, 39, 301, 302, 305, 306, 309, 310, 313, 112, 114, 183, 184, 405, 406, 475, and 476). Optionally, ALK7 polypeptides may comprise, consist essentially of, or consist of an amino acid sequence that is at least 70%, 80%, 85%, 90%, 95%, 97%, 98%, 99% or 100% identical to a polypeptide that begins at any one of amino acids 21-28 of SEQ ID NO: 38 (e.g., amino acids 21, 22, 23, 24, 25, 26, 27, or 28) and ends at any one of amino acids 92-113 of SEQ ID NO: 38 (e.g., amino acids 92, 93, 94, 95, 96, 97, 98, 99, 100, 101, 102, 103, 104, 105, 106, 107, 108, 109, 110, 111, 112, or 113 of SEQ ID NO: 38). Optionally, ALK7 polypeptides of the disclosure may be fusion proteins that further comprise one or more portions (domains) that are heterologous to ALK7. For example, an ALK7 polypeptide may be fused to a heterologous polypeptide that comprises a multimerization domain, optionally with a linker domain positioned between the ALK7 polypeptide and the heterologous polypeptide. In some embodiments, multimerization domains described herein comprise one component of an interaction pair. In some embodiments, heteromeric complexes that comprise an ALK7 polypeptide further comprise at least one different type I TGF-beta superfamily polypeptide. For example, an ALK7 heteromeric complex may further comprise an ALK2 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 18, 19, 136, 138, 173, 174, 421, 422, 465, and 466. In some embodiments, an ALK7 heteromeric complex may further comprise an ALK3 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 22, 23, 115, 117, 175, 176, 407, 408, 467, and 468. In some embodiments, an ALK7 heteromeric complex may further comprise an ALK4 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 26, 27, 83, 84, 104, 106, 177, 178, 403, 404, 469, and 470. In some embodiments, an ALK7 heteromeric complex may further comprise an ALK5 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 30, 31, 87, 88, 139, 141, 179, 180, 423, 424, 471, and 472. In some embodiments, an ALK7 heteromeric complex may further comprise an ALK6 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 34, 35, 91, 92, 142, 144, 181, 182, 425, 426, 473, and 474. In some embodiments, an ALK7 heteromeric complex may further comprise an ALK1 polypeptide as described herein, including, e.g., a polypeptide comprising, consisting essentially of, or consisting of an amino acid sequence that is at least 70%, 80%, 85%, 90%, 95%, 97%, 98%, 99% or 100% identical to a sequence selected from SEQ ID NOs: 14, 15, 124, 126, 171, 172, 413, 414, 463, and 464.

In some embodiments, the TGF-beta superfamily type I and/or type II receptor polypeptides disclosed herein comprise one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. In some embodiments, the TGF-beta superfamily type I and/or type II polypeptides described herein are glycosylated and have a glycosylation pattern obtainable from the expression of the polypeptides in a mammalian cell, including, for example, a CHO cell.

In certain aspects the disclosure provides nucleic acids encoding any of the TGF-beta superfamily type I and/or type II polypeptides described herein. Nucleic acids disclosed herein may be operably linked to a promoter for expression, and the disclosure further provides cells transformed with such recombinant polynucleotides. Preferably the cell is a mammalian cell such as a COS cell or a CHO cell.

In certain aspects, the disclosure provides methods for making any of the TGF-beta superfamily type I and/or type II polypeptides described herein as well as protein complexes comprising such polypeptides. Such a method may include expressing any of the nucleic acids disclosed herein in a suitable cell (e.g., CHO cell or a COS cell). Such a method may comprise: a) culturing a cell under conditions suitable for expression of the TGF-beta superfamily type I or type II polypeptides described herein, wherein said cell is transformed with a type I or type II polypeptide expression construct; and b) recovering the type I or type II polypeptides so expressed. TGF-beta superfamily type I and/or type II polypeptides described herein, as well as protein complexes of the same, may be recovered as crude, partially purified, or highly purified fractions using any of the well-known techniques for obtaining protein from cell cultures.

In certain aspects, the disclosure provides methods for making any of the heteromultimeric complexes disclosed herein. Such a method may include expressing any of the nucleic acids disclosed herein in a suitable cell (e.g., CHO cell or a COS cell). Such a method may comprise: a) obtaining a cell that comprises a nucleic acid comprising the coding sequence for a TGF-beta superfamily type I receptor polypeptide disclosed herein and a nucleic acid comprising the coding sequence for a TGF-beta superfamily type II receptor polypeptide disclosed herein; (b) culturing such cell under conditions suitable for expression of the TGF-beta superfamily type I and type II polypeptides described herein; and c) recovering the heteromeric complex comprising such type I and type II polypeptides so expressed. Heteromultimeric complexes disclosed herein as crude, partially purified, or highly purified fractions using any of the well-known techniques for obtaining protein from cell cultures.

Any of the protein complexes described herein may be incorporated into a pharmaceutical preparation. Optionally, such pharmaceutical preparations are at least 80%, 85%, 90%, 95%, 97%, 98% or 99% pure with respect to other polypeptide components. Optionally, pharmaceutical preparations disclosed herein may comprise one or more additional active agents. In some embodiments, heteromultimers of the disclosure comprise less than 10%, 9%, 8%, 7%, 5%, 4%, 3%, 2%, or less than 1% type I receptor polypeptide homomultimers. In some embodiments, heteromultimers of the disclosure comprise less than 10%, 9%, 8%, 7%, 5%, 4%, 3%, 2%, or less than 1% type II receptor polypeptide homomultimers. In some embodiments, heteromultimers of the disclosure comprise less than 10%, 9%, 8%, 7%, 5%, 4%, 3%, 2%, or less than 1% type I receptor polypeptide homomultimers and less than 10%, 9%, 8%, 7%, 5%, 4%, 3%, 2%, or less than 1% type II receptor polypeptide homomultimers.

The disclosure further provides methods and heteromultimeric complexes for use in the treatment or prevention of various disease and disorders associated with, for example, muscle, bone, fat, red blood cells, and other tissues that are affected by one or more ligands of the TGF-beta superfamily. Such disease and disorders include, but are not limited to, disorders associated with muscle loss or insufficient muscle growth (e.g., muscle atrophy; muscular dystrophy, including Duchenne muscular dystrophy, Becker muscular dystrophy, and facioscapulohumeral muscular dystrophy; amyotrophic lateral sclerosis; and cachexia) and disorders associated with undesirable weight gain (e.g., obesity, type 2 diabetes or non-insulin dependent diabetes mellitus (NIDDM), cardiovascular disease, hypertension, osteoarthritis, stroke, respiratory problems, and gall bladder disease). In some embodiments, heteromultimeric complexes disclosed herein may be used to decrease body fat content or reduce the rate of increase in body fat content in a subject in need thereof. In some embodiments, heteromultimeric complexes disclosed herein may be used to reduce cholesterol and/or triglyceride levels in a patient.

### BRIEF DESCRIPTION OF THE DRAWINGS

FIGS. 1A and 1B show two schematic examples of heteromeric protein complexes comprising type I receptor and type II receptor polypeptides. FIG. 1A depicts a heterodimeric protein complex comprising one type I receptor fusion polypeptide and one type II receptor fusion polypeptide, which can be assembled covalently or noncovalently via a multimerization domain contained within each polypeptide chain. Two assembled multimerization domains constitute an interaction pair, which can be either guided or unguided. FIG. 2A depicts a heterotetrameric protein complex comprising two heterodimeric complexes as in FIG. 1A. Complexes of higher order can be envisioned.

FIG. 2 shows a schematic example of a heteromeric protein complex comprising a type I receptor polypeptide (indicated as “I”) (e.g. a polypeptide that is at least 80%, 85%, 90%, 95%, 97%, 98%, 99% or 100% identical to an extracellular domain of an ALK1, ALK2, ALK3, ALK4, ALK5, ALK6 or ALK7 protein from humans or other species such as those described herein, e.g., SEQ ID Nos: 14, 15, 124, 126, 171, 172, 413, 414, 463, 464, 18, 19, 136, 138, 173, 174, 421, 422, 465, 466, 22, 23, 115, 117, 175, 176, 407, 408, 467, 468, 26, 27, 83, 84, 104, 106, 177, 178, 403, 404, 469, 470, 30, 31, 87, 88, 139, 141, 179, 180, 423, 424, 471, 472, 34, 35, 91, 92, 142, 144, 181, 182, 425, 426, 473, 474, 38, 39, 301, 302, 305, 306, 309, 310, 313, 112, 114, 183, 184, 405, 406, 475, and 476) and a type II receptor polypeptide (indicated as “II”) (e.g. a polypeptide that is at least 80%, 85%, 90%, 95%, 97%, 98%, 99% or 100% identical to an extracellular domain of an ActRIIA, ActRIIB, MISRII, BMPRII, or TGFBRII protein from humans or other species such as those described herein, e.g., 9, 10, 11, 118, 120, 151, 152, 409, 410, 451, 452, 1, 2, 3, 4, 5, 6, 100, 102, 153, 154, 401, 402, 453, 454, 46, 47, 71, 72, 121, 123, 155, 156, 411, 412, 455, 456, 50, 51, 75, 76, 79, 80, 133, 135, 161, 162, 419, 420, 457, 458, 42, 43, 67, 68, 127, 129, 130, 132, 157, 158, 159, 160, 415, 416, 417, 418, 459, 460, 461, and 462). In the illustrated embodiment, the type I receptor polypeptide is part of a fusion polypeptide that comprises a first member of an interaction pair (“C”), and the type II receptor polypeptide is part of a fusion polypeptide that comprises a second member of an interaction pair (“D”). In each fusion polypeptide, a linker may be positioned between the type I or type II receptor polypeptide and the corresponding member of the interaction pair. The first and second members of the interaction pair (C, D) may be a guided (asymmetric) pair, meaning that the members of the pair associate preferentially with each other rather than self-associate, or the interaction pair may be unguided, meaning that the members of the pair may associate with each other or self-associate without substantial preference and may have the same or different amino acid sequences. Traditional Fc fusion proteins and antibodies are examples of unguided interaction pairs, whereas a variety of engineered Fc domains have been designed as guided (asymmetric) interaction pairs [e.g., Spiess et al (2015) Molecular Immunology 67(2A): 95-106].

FIG. 3 shows an alignment of extracellular domains of human ActRIIA (SEQ ID NO: 500) and human ActRIIB (SEQ ID NO: 2) with the residues that are deduced herein, based on composite analysis of multiple ActRIIB and ActRIIA crystal structures, to directly contact ligand indicated with boxes.

FIG. 4 shows a multiple sequence alignment of various vertebrate ActRIIB precursor proteins without their intracellular domains (SEQ ID NOs: 501, 502, 503, 504, 505, and 506, respectively), human ActRIIA precursor protein without its intracellular domain (SEQ ID NO: 507), and a consensus ActRII precursor protein (SEQ ID NO: 508).

FIG. 5 shows multiple sequence alignment of Fc domains from human IgG isotypes using Clustal 2.1 (SEQ ID NOs: 208, 212, 209 and 210, respectively, in order of appearance). Hinge regions are indicated by dotted underline. Double underline indicates examples of positions engineered in IgG1 Fe to promote asymmetric chain pairing and the corresponding positions with respect to other isotypes IgG2, IgG3 and IgG4.

FIG. 6 shows ligand binding data for an ActRIIB-Fc:ALK4-Fc heterodimeric protein complex as compared to ActRIIB-Fc homodimer and ALK4-Fc homodimer. For each protein complex, ligands are ranked by k_{off}, a kinetic constant that correlates well with ligand signaling inhibition, and listed in descending order of binding affinity (ligands bound most tightly are listed at the top). At left, yellow, red, green, and blue lines indicate magnitude of the off-rate constant. Solid black lines indicate ligands whose binding to heterodimer is enhanced or unchanged compared with homodimer, whereas dashed red lines indicate substantially reduced binding compared with homodimer. As shown, the ActRIIB-Fc:ALK4-Fc heterodimer displays enhanced binding to activin B compared with either homodimer, retains strong binding to activin A, GDF8, and GDF11 as observed with ActRIIB-Fc homodimer, and exhibits substantially reduced binding to BMP9, BMP10, and GDF3. Like ActRIIB-Fc homodimer, the heterodimer retains intermediate-level binding to BMP6.

FIG. 7 shows ligand binding data for an ActRIIB-Fc:ALK3-Fc heterodimeric protein complex as compared to ActRIIB-Fc homodimer and ALK3-Fc homodimer. Format is the same as in FIG. 6. As shown, the ActRIIB-Fc:ALK3-Fc heterodimer binds BMP2 and BMP4 with exceptionally high affinity and displays greatly enhanced binding to BMP5, BMP6, BMP7, GDF5, GDF6, and GDF7 compared with either homodimer. Compared to ActRIIB homodimer, the ActRIIB-Fc:ALK3-Fc heterodimer displays reduced binding to activin A, activin B, BMP10, GDF8, and GDF11 and also discriminates among these ligands to a greater degree, particularly between activin A and activin B. In addition, the ability of ActRIIB-Fc homodimer to bind BMP9 and GDF3 with high affinity is absent for ActRIIB-Fc:ALK3-Fc heterodimer.

FIG. 8 shows ligand binding data for an ActRIIB-Fc:ALK7-Fc heterodimeric protein complex as compared to ActRIIB-Fc homodimer and ALK7-Fc homodimer. Format is the same as in FIG. 6. As shown, four of the five ligands with strong binding to ActRIIB-Fc homodimer (activin A, BMP10, GDF8, and GDF11) exhibit reduced binding to the ActRIIB-Fc:ALK7-Fc heterodimer, the exception being activin B which retains tight binding to the heterodimer. In addition, three ligands with intermediate binding to ActRIIB-Fc homodimer (GDF3, BMP6, and particularly BMP9) exhibit reduced binding to the ActRIIB-Fc:ALK7-Fc heterodimer. In contrast, BMP5 binds the ActRIIB-Fc:ALK7 heterodimer with intermediate strength despite only weak binding to ActRIIB-Fc homodimer. No ligands tested bind to ALK7-Fc homodimer.

FIG. 9 shows ligand binding data for an ActRIIB-Fc:ALK2-Fc heterodimeric protein complex as compared to ActRIIB-Fc homodimer and ALK2-Fc homodimer. Format is the same as in FIG. 6. As shown, the ActRIIB-Fc:ALK2-Fc heterodimer exhibits preferential and strong binding to activin B, thus resembling ActRIIB-Fc:ALK7-Fc heterodimer (FIG. 8). However, ActRIIB-Fc:ALK2-Fc heterodimer differs from ActRIIB-Fc:ALK7-Fc in part by retaining the tight binding to BMP9 characteristic of ActRIIB-Fc homodimer. No ligands tested bind to ALK2-Fc homodimer.

FIG. 10 shows ligand binding data for an ActRIIA-Fc:ALK4-Fc heterodimeric protein complex as compared to ActRIIA-Fc homodimer and ALK4-Fc homodimer. Format is the same as in FIG. 6. As shown, the ActRIIA-Fc:ALK4-Fc heterodimer exhibits enhanced binding to activin A, and particularly enhanced binding to activin AC, compared to ActRIIA-Fc homodimer, while retaining strong binding to activin AB and GDF11. In addition, the ligand with highest affinity for ActRIIA-Fc homodimer, activin B, displays reduced affinity (albeit still within the high-affinity range) for the ActRIIA-Fc:ALK4-Fc heterodimer. The ActRIIA-Fc:ALK4-Fc heterodimer also exhibits markedly reduced binding to BMP10 compared to ActRIIA-Fc homodimer.

FIG. 11 shows ligand binding data for a BMPRII-Fc:ALK1-Fc heterodimeric protein complex as compared to ActRIIB-Fc homodimer and ALK1-Fc homodimer. Format is the same as in FIG. 6. As shown, the BMPRII-Fc:ALK1-Fc heterodimer largely retains the strong binding to BMP9 and BMP10 characteristic of ALK1-Fc homodimer; however, the heterodimer displays modest selectivity for BMP10 over BMP9 not present with the homodimer. Also unlike ALK1-Fc homodimer, the BMPRII-Fc:ALK1-Fc heterodimer binds to BMP15, albeit with an off-rate approximately ten times faster than that of BMPRII-Fc homodimer.

FIG. 12 shows ligand binding data for a BMPRII-Fc:ALK3-Fc heterodimeric protein complex as compared to BMPRII-Fc homodimer and ALK3-Fc homodimer. Format is the same as in FIG. 6. As shown, the BMPRII-Fc:ALK3-Fc heterodimer binds much more strongly to BMP6 than does ALK3-Fc homodimer, reflecting an off-rate nearly ten times slower. With its largely unchanged binding to BMP2 and BMP4, the BMPRII-Fc:ALK3 heterodimer can therefore be considered a joint inhibitor of BMP2, BMP4, and BMP6. This binding profile contrasts with that of ALK3-Fc homodimer, whose exceptionally strongly binding to BMP4 and BMP2 identifies it as highly selective for this ligand pair compared to four ligands with intermediate-level binding, including BMP6.

FIG. 13 shows ligand binding data for a BMPRII-Fc:ALK4-Fc heterodimeric protein complex as compared to BMPRII-Fc homodimer and ALK4-Fc homodimer. Format is the same as in FIG. 6. BMPRII-Fc:ALK4-Fc heterodimer differs from both homodimers by binding several activin ligands with high or intermediate strength and differs from BMPRII-Fc homodimer by binding BMP15 only weakly. Most notably, BMPRII-Fc:ALK4-Fc heterodimer binds strongly and with high selectivity to the heterodimeric ligand activin AB.

FIG. 14 shows ligand binding data for two different TGFβRII-Fc:ALK5-Fc heterodimeric protein complexes as compared to TGFβRII-Fc homodimer and ALK5-Fc homodimer. Format is the same as in FIG. 6. As shown, TGFβRII-Fc:ALK5-Fc heterodimers differ markedly from TGFβRII-Fc homodimer in their high selectivity for TGFβ2 while still retaining considerable affinity for TGFβ1 and TGFβ3. The heterodimer incorporating the long isoform of TGFβRII bound TGFβ2 more strongly and selectively than did its short-isoform counterpart. No ligands tested bind to ALK5-Fc homodimer.

FIGS. 15A-15D show schematic examples of heteromeric protein complexes comprising a type I receptor polypeptide (indicated as “I”) (e.g. a polypeptide that is at least 80%, 85%, 90%, 95%, 97%, 98%, 99% or 100% identical to an extracellular domain of an ALK1, ALK2, ALK3, ALK4, ALK5, ALK6 or ALK7 protein from humans or other species such as those described herein, e.g., SEQ ID Nos: 14, 15, 124, 126, 171, 172, 413, 414, 463, 464, 18, 19, 136, 138, 173, 174, 421, 422, 465, 466, 22, 23, 115, 117, 175, 176, 407, 408, 467, 468, 26, 27, 83, 84, 104, 106, 177, 178, 403, 404, 469, 470, 30, 31, 87, 88, 139, 141, 179, 180, 423, 424, 471, 472, 34, 35, 91, 92, 142, 144, 181, 182, 425, 426, 473, 474, 38, 39, 301, 302, 305, 306, 309, 310, 313, 112, 114, 183, 184, 405, 406, 475, and 476) and a type II receptor polypeptide (indicated as “II”) (e.g. a polypeptide that is at least 80%, 85%, 90%, 95%, 97%, 98%, 99% or 100% identical to an extracellular domain of an ActRIIA, ActRIIB, MISRII, BMPRII, or TGFBRII protein from humans or other species such as those described herein, e.g., 9, 10, 11, 118, 120, 151, 152, 409, 410, 451, 452, 1, 2, 3, 4, 5, 6, 100, 102, 153, 154, 401, 402, 453, 454, 46, 47, 71, 72, 121, 123, 155, 156, 411, 412, 455, 456, 50, 51, 75, 76, 79, 80, 133, 135, 161, 162, 419, 420, 457, 458, 42, 43, 67, 68, 127, 129, 130, 132, 157, 158, 159, 160, 415, 416, 417, 418, 459, 460, 461, and 462). In the illustrated embodiments, the a type I receptor polypeptide is part of a fusion polypeptide that comprises a first member of an interaction pair (“C₁”), and a type II receptor polypeptide is part of a fusion polypeptide that comprises a second member of an interaction pair (“C₂”). Suitable interaction pairs included, for example, heavy chain and/or light chain immunoglobulin interaction pairs, truncations, and variants thereof such as those described herein [e.g., Spiess et al (2015) Molecular Immunology 67(2A): 95-106]. In each fusion polypeptide, a linker may be positioned between the a type I receptor polypeptide or a type II receptor polypeptide and the corresponding member of the interaction pair. The first and second members of the interaction pair may be unguided, meaning that the members of the pair may associate with each other or self-associate without substantial preference, and they may have the same or different amino acid sequences. See FIG. 15A. Alternatively, the interaction pair may be a guided (asymmetric) pair, meaning that the members of the pair associate preferentially with each other rather than self-associate. See FIG. 15B. Complexes of higher order can be envisioned. See FIGS. 15C and 15D.

FIGS. 16A-16G show schematic examples of heteromeric protein complexes comprising two type I receptor polypeptide (indicated as “I”) (e.g. a polypeptide that is at least 80%, 85%, 90%, 95%, 97%, 98%, 99% or 100% identical to an extracellular domain of an ALK1, ALK2, ALK3, ALK4, ALK5, ALK6 or ALK7 protein from humans or other species such as those described herein, e.g., SEQ ID Nos: 14, 15, 124, 126, 171, 172, 413, 414, 463, 464, 18, 19, 136, 138, 173, 174, 421, 422, 465, 466, 22, 23, 115, 117, 175, 176, 407, 408, 467, 468, 26, 27, 83, 84, 104, 106, 177, 178, 403, 404, 469, 470, 30, 31, 87, 88, 139, 141, 179, 180, 423, 424, 471, 472, 34, 35, 91, 92, 142, 144, 181, 182, 425, 426, 473, 474, 38, 39, 301, 302, 305, 306, 309, 310, 313, 112, 114, 183, 184, 405, 406, 475, and 476) and two type II receptor polypeptide (indicated as “II”) (e.g. a polypeptide that is at least 80%, 85%, 90%, 95%, 97%, 98%, 99% or 100% identical to an extracellular domain of an ActRIIA, ActRIIB, MISRII, BMPRII, or TGFBRII protein from humans or other species such as those described herein, e.g., 9, 10, 11, 118, 120, 151, 152, 409, 410, 451, 452, 1, 2, 3, 4, 5, 6, 100, 102, 153, 154, 401, 402, 453, 454, 46, 47, 71, 72, 121, 123, 155, 156, 411, 412, 455, 456, 50, 51, 75, 76, 79, 80, 133, 135, 161, 162, 419, 420, 457, 458, 42, 43, 67, 68, 127, 129, 130, 132, 157, 158, 159, 160, 415, 416, 417, 418, 459, 460, 461, and 462).

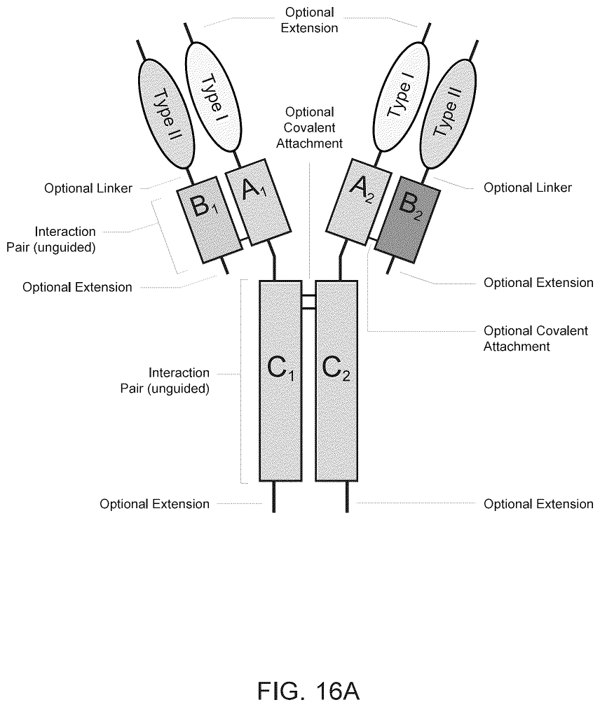

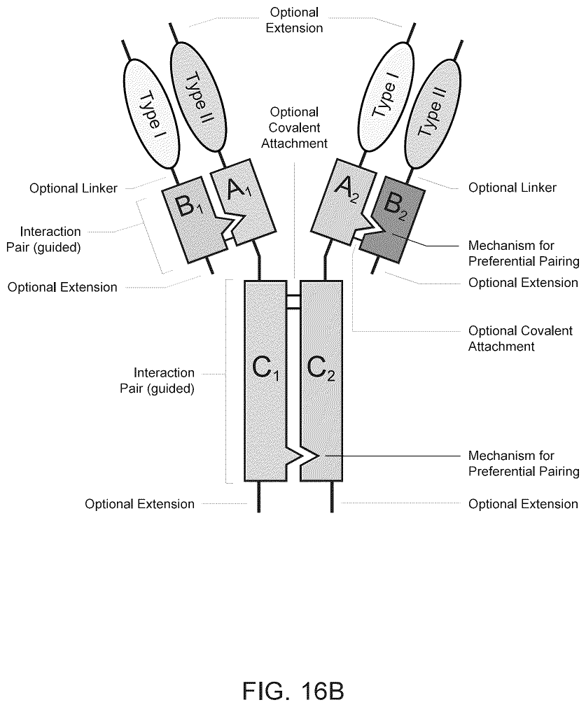

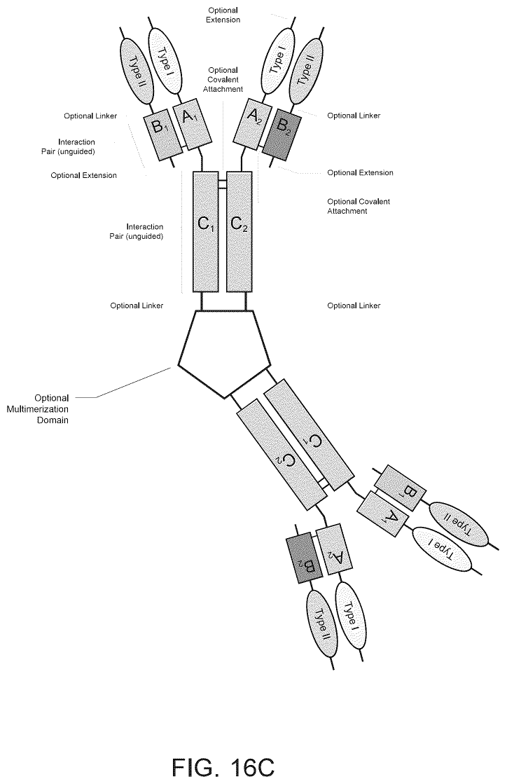

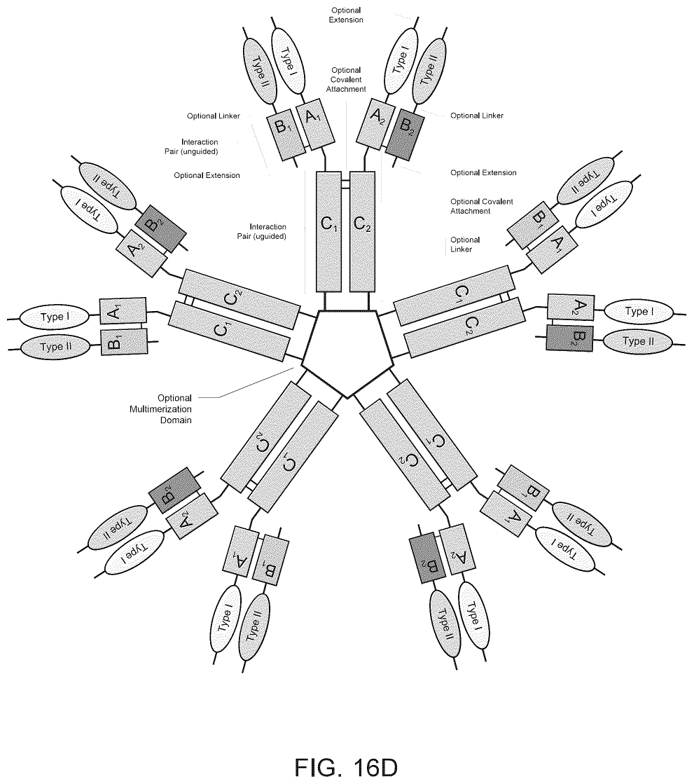

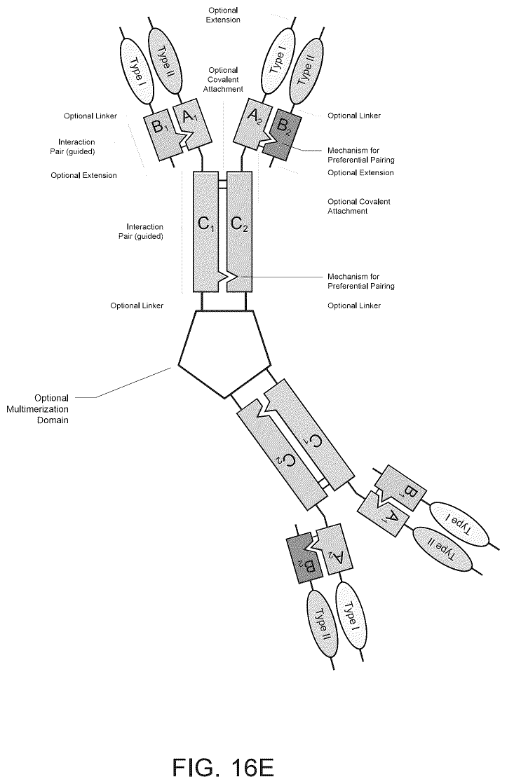

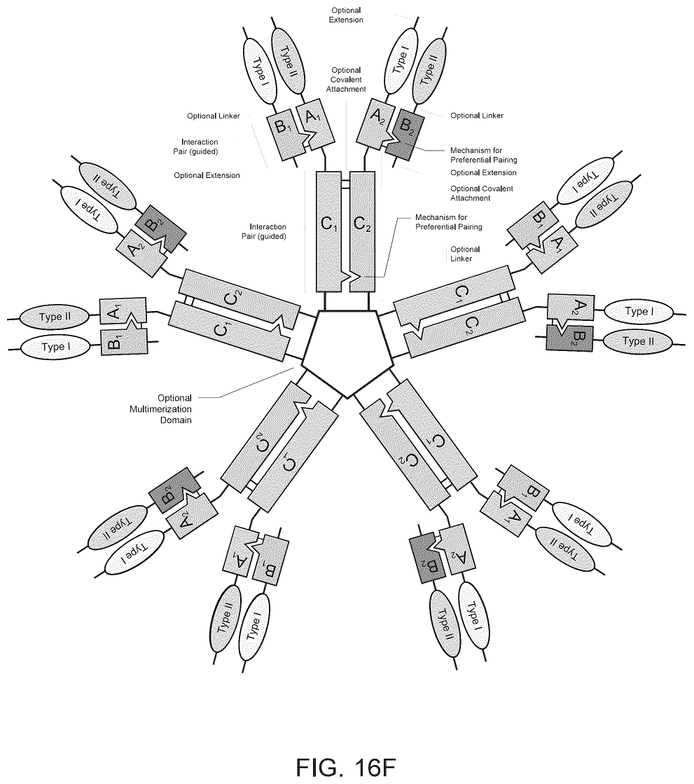

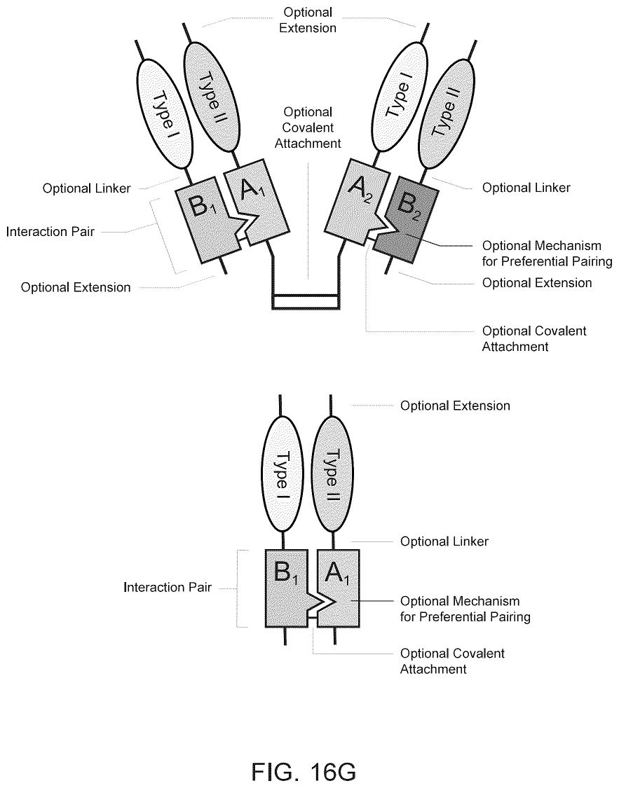

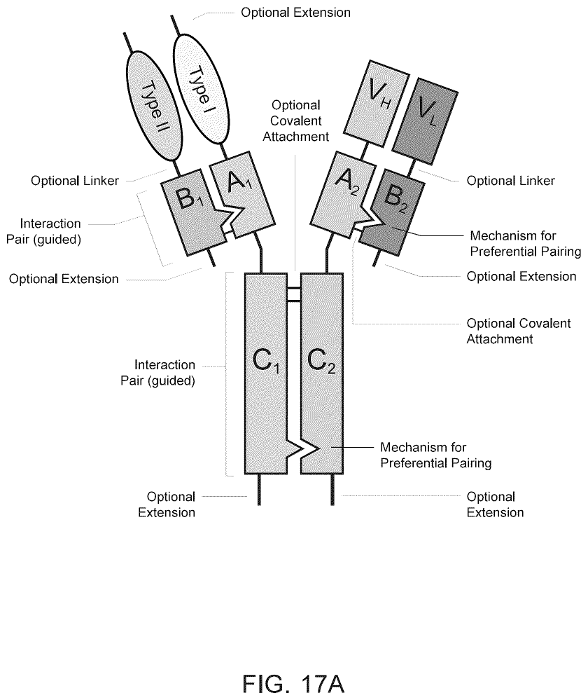

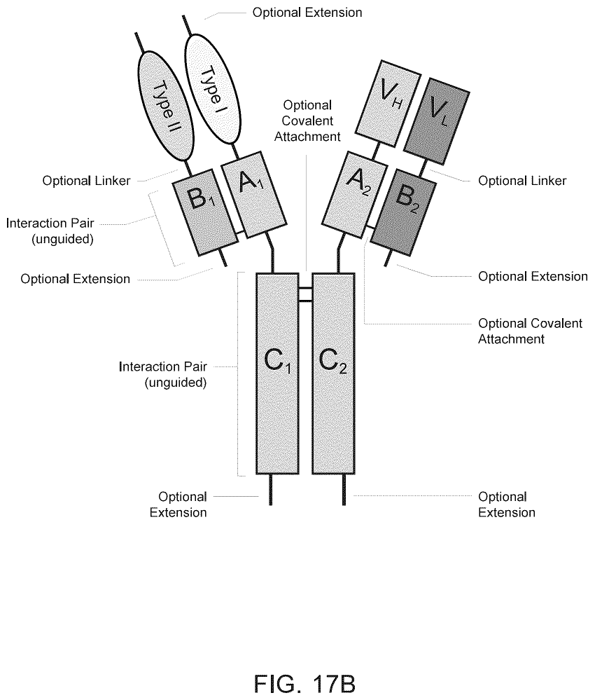

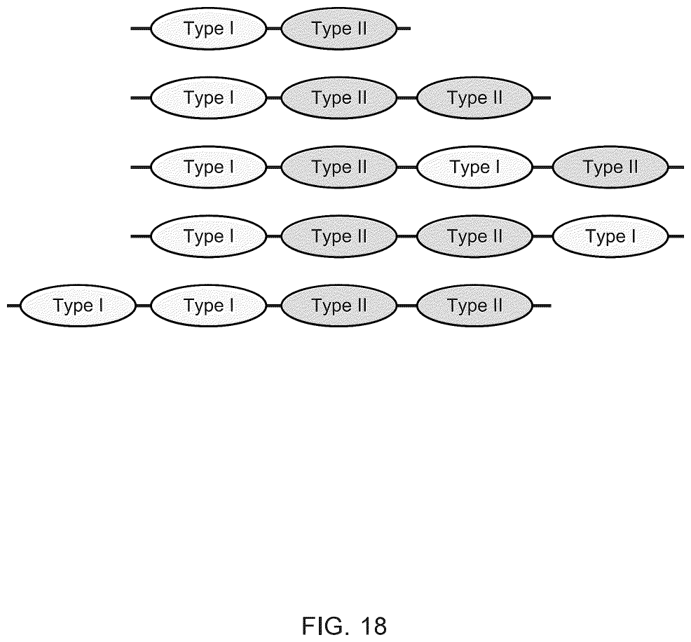

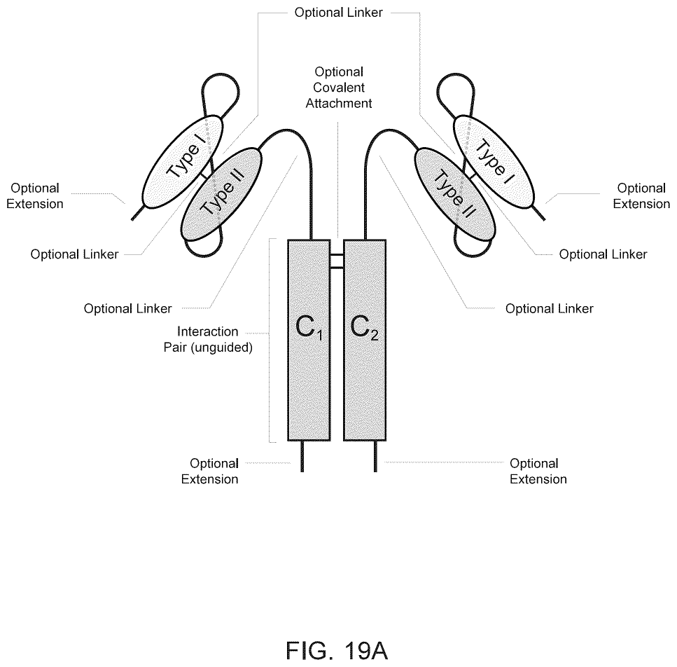

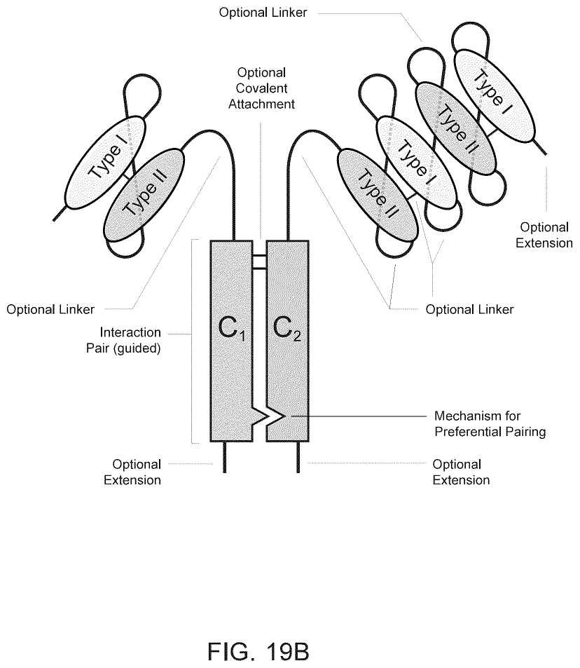

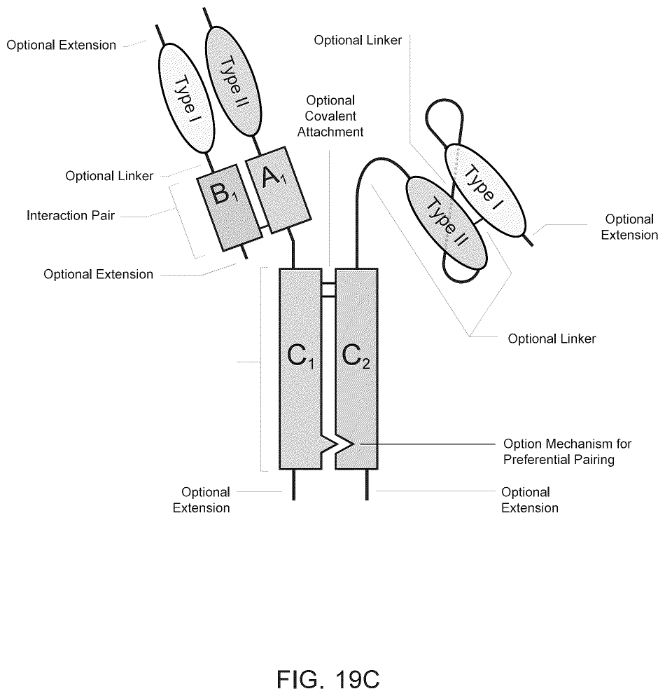

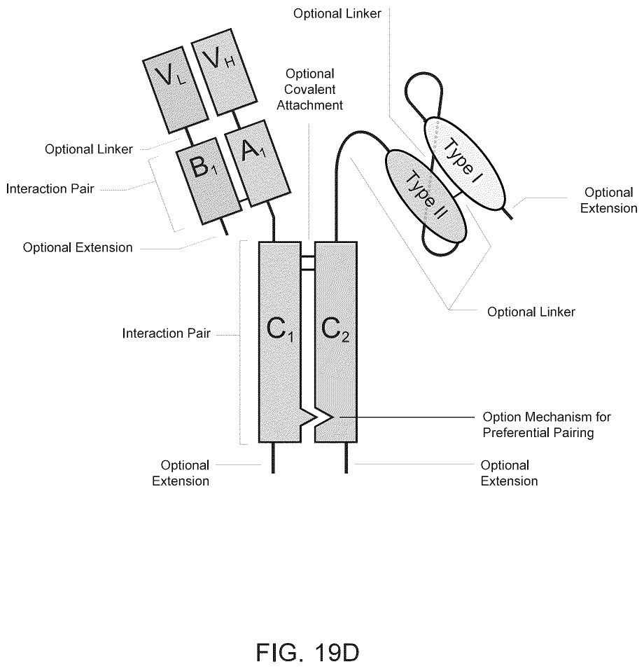

In the illustrated embodiment FIG. 16A, the first type I receptor polypeptide (from left to right) is part of a fusion polypeptide that comprises a first member of an interaction pair (“C₁”) and further comprises an additional first member of an interaction pair (“A₁”); and the second type I receptor polypeptide is part of a fusion polypeptide that comprises a second member of an interaction pair (“C₂”) and further comprises an first member of an interaction pair (“A₂”). The first type II receptor polypeptide (from left to right) is part of a fusion polypeptide that comprises a second member of an interaction pair (“B₁”); and the second type II receptor polypeptide is part of a fusion polypeptide that comprises a second member of an interaction pair (“B₂”). A₁ and A₂ may be the same or different; B₁ and B₂ may be the same or different, and C₁ and C₂ may be the same or different. In each fusion polypeptide, a linker may be positioned between the type I receptor polypeptide or type II receptor polypeptide and the corresponding member of the interaction pair as well as between interaction pairs. FIG. 16A is an example of an association of unguided interaction pairs, meaning that the members of the pair may associate with each other or self-associate without substantial preference and may have the same or different amino acid sequences.

In the illustrated embodiment FIG. 16B, the first type II receptor polypeptide (from left to right) is part of a fusion polypeptide that comprises a first member of an interaction pair (“C₁”) and further comprises an additional first member of an interaction pair (“A₁”); and the second type II receptor ActRIIB polypeptide is part of a fusion polypeptide that comprises a second member of an interaction pair (“B₂”). The first type I receptor polypeptide (from left to right) is part of a fusion polypeptide that comprises a second member of an interaction pair (“B₁”); and the second type I receptor polypeptide is part of a fusion polypeptide that comprises a second member of an interaction pair (“C₂”) and further comprises a first member of an interaction pair (“A₂”). In each fusion polypeptide, a linker may be positioned between the type I receptor or type II receptor polypeptide and the corresponding member of the interaction pair as well as between interaction pairs. FIG. 16B is an example of an association of guided (asymmetric) interaction pairs, meaning that the members of the pair associate preferentially with each other rather than self-associate.

Suitable interaction pairs included, for example, heavy chain and/or light chain immunoglobulin interaction pairs, truncations, and variants thereof as described herein [e.g., Spiess et al (2015) Molecular Immunology 67(2A): 95-106]. Complexes of higher order can be envisioned. See FIGS. 16C-16F. Using similar methods, particularly those that employ light and/or heavy chain immunoglobulins, truncations, or variants thereof, interaction pairs may be used to produce heterodimers that resemble antibody Fab and F(ab′)₂ complexes [e.g., Spiess et al (2015) Molecular Immunology 67(2A): 95-106]. See FIG. 16G.

FIGS. 17A and 17B show schematic examples of a heteromeric protein complex comprising a type I receptor polypeptide (indicated as “I”) (e.g. a polypeptide that is at least 80%, 85%, 90%, 95%, 97%, 98%, 99% or 100% identical to an extracellular domain of an ALK1, ALK2, ALK3, ALK4, ALK5, ALK6 or ALK7 protein from humans or other species such as those described herein, e.g., SEQ ID Nos: 14, 15, 124, 126, 171, 172, 413, 414, 463, 464, 18, 19, 136, 138, 173, 174, 421, 422, 465, 466, 22, 23, 115, 117, 175, 176, 407, 408, 467, 468, 26, 27, 83, 84, 104, 106, 177, 178, 403, 404, 469, 470, 30, 31, 87, 88, 139, 141, 179, 180, 423, 424, 471, 472, 34, 35, 91, 92, 142, 144, 181, 182, 425, 426, 473, 474, 38, 39, 301, 302, 305, 306, 309, 310, 313, 112, 114, 183, 184, 405, 406, 475, and 476) and a type II receptor polypeptide (indicated as “II”) (e.g. a polypeptide that is at least 80%, 85%, 90%, 95%, 97%, 98%, 99% or 100% identical to an extracellular domain of an ActRIIA, ActRIIB, MISRII, BMPRII, or TGFBRII protein from humans or other species such as those described herein, e.g., 9, 10, 11, 118, 120, 151, 152, 409, 410, 451, 452, 1, 2, 3, 4, 5, 6, 100, 102, 153, 154, 401, 402, 453, 454, 46, 47, 71, 72, 121, 123, 155, 156, 411, 412, 455, 456, 50, 51, 75, 76, 79, 80, 133, 135, 161, 162, 419, 420, 457, 458, 42, 43, 67, 68, 127, 129, 130, 132, 157, 158, 159, 160, 415, 416, 417, 418, 459, 460, 461, and 462), and a ligand-binding domain of an antibody (e.g., a ligand binding domain derived form an antibody that binds to one or more TGF-beta superfamily ligands). In the illustrated embodiments, the type I receptor polypeptide is part of a fusion polypeptide that comprises a first member of an interaction pair (“C₁”), and further comprises an additional first member of an interaction pair (“A₁”). The type II receptor polypeptide is part of a fusion polypeptide that comprises a second member of an interaction pair (“B₁”). The variable heavy chain (V_{H}) polypeptide is part of a fusion polypeptide that comprises a second member of an interaction pair (“C₂”), and further comprises a first member of an interaction pair (“A₂”). The variable heavy chain (V_{L}) polypeptide is part of a fusion polypeptide that comprises a second member of an interaction pair (“B₂”). In each fusion polypeptide, a linker may be positioned between the type I receptor or type II receptor polypeptide and the corresponding member of the interaction pair, between interaction pairs, and between the V_{H} and V_{L} polypeptides and a member of the interaction pair. A₁ and A₂ may be the same or different; B₁ and B₂ may be the same or different, and C₁ and C₂ may be the same or different. Suitable interaction pairs included, for example, constant heavy chain and/or light chain immunoglobulin interaction pairs, truncations, and variants thereof as described herein [e.g., Spiess et al (2015) Molecular Immunology 67(2A): 95-106]. FIG. 17A is an example of an association of guided (asymmetric) interaction pairs, meaning that the members of the pair associate preferentially with each other rather than self-associate. FIG. 17B is an example of an association of unguided interaction pairs, meaning that the members of the pair may associate with each other or self-associate without substantial preference and may have the same or different amino acid sequences.

FIG. 18 shows schematic examples of type I receptor: type II receptor single-trap polypeptides. Type I receptor: type II receptor single-trap polypeptides may contain multiple type I receptor domains (e.g., 1, 2, 3, 4, 5, 6, 7, 9, 10 or more domains), having the same or different sequences, and multiple type II receptor domains (e.g., 1, 2, 3, 4, 5, 6, 7, 9, 10 or more domains), having the same or different sequences. These type I receptor and type II receptor domains may be arranged in any order and may comprise one or more linker domains positions between one or more of the type II receptor and type II receptor domains. Such ligand traps may be useful as therapeutic agents to treat or prevent diseases or disorders described herein.

FIGS. 19A-19D show schematic examples of multimeric protein complexes comprising at least one type I receptor: type II receptor single-chain trap polypeptide. In the illustrated embodiments 19A and 19B, a first type I receptor: type II receptor single-chain trap polypeptide (from left to right) is part of a fusion polypeptide that comprises a first member of an interaction pair (“C₁”); and a second type I receptor: type II receptor single-chain trap polypeptide is part of a fusion polypeptide that comprises a second member of an interaction pair (“C₂”). C₁ and C₂ may be the same or different. The first and second type I receptor: type II receptor single-chain trap polypeptides may be the same or different. In each fusion polypeptide, a linker may be positioned between the type I receptor: type II receptor single-chain trap polypeptide and the corresponding member of the interaction pair. Suitable interaction pairs included, for example, heavy chain and/or light chain immunoglobulin interaction pairs, truncations, and variants thereof as described herein [e.g., Spiess et al (2015) Molecular Immunology 67(2A): 95-106]. FIG. 19A is an example of an association of unguided interaction pairs, meaning that the members of the pair may associate with each other or self-associate without substantial preference and may have the same or different amino acid sequences. FIG. 19B is an example of an association of guided (asymmetric) interaction pairs, meaning that the members of the pair associate preferentially with each other rather than self-associate. Complexes of higher order can be envisioned. In addition, such type I receptor: type II receptor single-chain trap polypeptides may be similarly be associated, covalently or non-covalently, with one or more type I receptor polypeptides and/or one or more type II receptor polypeptides. See FIG. 19C. Also, such type I receptor: type II receptor single-chain trap polypeptides may be similarly be associated, covalently or non-covalently, with one or more ligand-binding domain of an antibody (e.g., a ligand binding domain of an antibody that binds to one or more type I receptor: type II receptor heteromultimer binding-ligands). See FIG. 19D.

### DETAILED DESCRIPTION OF THE INVENTION

### 1. Overview

In part, the present disclosure relates to heteromultimer complexes comprising an extracellular domain of a TGFβ superfamily type I receptor polypeptide and an extracellular domain of a TGFβ superfamily type II receptor polypeptide, heteromultimer complexes comprising an extracellular domain of at least two different TGFβ superfamily type I receptor polypeptides, heteromultimer complexes comprising an extracellular domain of at least two different TGFβ superfamily type II receptor polypeptides, methods of making such heteromultimer complexes, and uses thereof. As described herein, in some embodiments, heteromultimer complexes may comprise an extracellular domain of a TGFβ superfamily type I receptor polypeptide selected from: ALK1, ALK2, ALK3, ALK4, ALK5, ALK6, and ALK7. Similarly, in some embodiments, these heteromultimer complexes may comprise an extracellular domain of a TGFβ superfamily type II receptor polypeptide selected from: ActRIIA, ActRIIB, TGFBRII, BMPRII, and MISRII. In certain preferred embodiments, heteromultimer complexes of the disclosure have an altered TGFβ superfamily ligand binding specificity/profile relative to a corresponding sample of a homomultimer complex (e.g., an ActRIIB:ALK4 heterodimer compared to an ActRIIB:ActRIIB homodimer complex or an ALK4:ALK4 homodimer complex).

The TGF-β superfamily is comprised of over 30 secreted factors including TGF-betas, activins, nodals, bone morphogenetic proteins (BMPs), growth and differentiation factors (GDFs), and anti-Mullerian hormone (AMH). See, e.g., Weiss et al. (2013) Developmental Biology, 2(1): 47-63. Members of the superfamily, which are found in both vertebrates and invertebrates, are ubiquitously expressed in diverse tissues and function during the earliest stages of development throughout the lifetime of an animal. Indeed, TGF-β superfamily proteins are key mediators of stem cell self-renewal, gastrulation, differentiation, organ morphogenesis, and adult tissue homeostasis. Consistent with this ubiquitous activity, aberrant TGF-beta superfamily signaling is associated with a wide range of human pathologies including, for example, autoimmune disease, cardiovascular disease, fibrotic disease, and cancer.

Ligands of the TGF-beta superfamily share the same dimeric structure in which the central 3-1/2 turn helix of one monomer packs against the concave surface formed by the beta-strands of the other monomer. The majority of TGF-beta family members are further stabilized by an intermolecular disulfide bonds. This disulfide bond traverses through a ring formed by two other disulfide bonds generating what has been termed a ‘cysteine knot’ motif See, e.g., Lin et al., (2006) Reproduction 132: 179-190 and Hinck et al. (2012) FEBS Letters 586: 1860-1870.

TGF-beta superfamily signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation. See, e.g., Massagué (2000) Nat. Rev. Mol. Cell Biol. 1:169-178. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

The TGF-beta family can be divided into two phylogenetic branches based on the type I receptors they bind and the Smad proteins they activate. One is the more recently evolved branch, which includes, e.g., the TGF-betas, activins, GDF8, GDF9, GDF11, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through Smads 1, 5, and 8.

TGF-beta isoforms are the founding members of the TGF-beta superfamily, of which there are 3 known isoforms in mammals designated as TGF-beta1, TGF-beta2 and TGF-beta3. Mature bioactive TGF-beta ligands function as homodimers and predominantly signal through the type I receptor ALK5, but have also been found to additionally signal through ALK1 in endothelial cells. See, e.g., Goumans et al. (2003) Mol Cell 12(4): 817-828. TGF-beta1 is the most abundant and ubiquitously expressed isoform. TGF-beta1 is known to have an important role in wound healing, and mice expressing a constitutively active TGF-beta1 transgene develop fibrosis. See e.g., Clouthier et al., (1997) J Clin. Invest. 100(11): 2697-2713. TGF-beta1 is also involved in T cell activation and maintenance of T regulatory cells. See, e.g., Li et al., (2006) Immunity 25(3): 455-471. TGF-beta2 expression was first described in human glioblastoma cells, and is occurs in neurons and astroglial cells of the embryonic nervous system. TGF-beta2 is known to suppress interleukin-2-dependent growth of T lymphocytes. TGF-beta3 was initially isolated from a human rhabdomyosarcoma cell line and since has been found in lung adenocarcinoma and kidney carcinoma cell lines. TGF-beta3 is known to be important for palate and lung morphogenesis. See, e.g., Kubiczkova et al., (2012) Journal of Translational Medicine 10:183.

Activins are members of the TGF-beta superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos. See, e.g., DePaolo et al. (1991) Proc Soc Ep Biol Med. 198:500-512; Dyson et al. (1997) Curr Biol. 7:81-84; and Woodruff (1998) Biochem Pharmacol. 55:953-963. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

As described herein, agents that bind to “activin A” are agents that specifically bind to the β_{A} subunit, whether in the context of an isolated β_{A} subunit or as a dimeric complex (e.g., a β_{A}β_{A} homodimer or a β_{A}β_{B} heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the RB subunit of the complex). Similarly, agents disclosed herein that antagonize (inhibit) “activin A” are agents that inhibit one or more activities as mediated by a β_{A} subunit, whether in the context of an isolated β_{A} subunit or as a dimeric complex (e.g., a β_{A}β_{A} homodimer or a β_{A}β_{B} heterodimer). In the case of β_{A}β_{B} heterodimers, agents that inhibit “activin A” are agents that specifically inhibit one or more activities of the β_{A} subunit, but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB”, “activin AC”, “activin AE”, “activin BC”, or “activin BE” are agents that inhibit one or more activities as mediated by the β_{A} subunit and one or more activities as mediated by the β_{B} subunit. The same principle applies to agents that bind to and/or inhibit “activin AC”, “activin AE”, “activin BC”, or “activin BE”.

Nodal proteins have functions in mesoderm and endoderm induction and formation, as well as subsequent organization of axial structures such as heart and stomach in early embryogenesis. It has been demonstrated that dorsal tissue in a developing vertebrate embryo contributes predominantly to the axial structures of the notochord and pre-chordal plate while it recruits surrounding cells to form non-axial embryonic structures. Nodal appears to signal through both type I and type II receptors and intracellular effectors known as SMAD proteins. Studies support the idea that ActRIIA and ActRIIB serve as type II receptors for nodal. See, e.g., Sakuma et al. (2002) Genes Cells. 2002, 7:401-12. It is suggested that Nodal ligands interact with their co-factors (e.g., Cripto or Cryptic) to activate activin type I and type II receptors, which phosphorylate SMAD2. Nodal proteins are implicated in many events critical to the early vertebrate embryo, including mesoderm formation, anterior patterning, and left-right axis specification. Experimental evidence has demonstrated that nodal signaling activates pAR3-Lux, a luciferase reporter previously shown to respond specifically to activin and TGF-beta. However, nodal is unable to induce pT1x2-Lux, a reporter specifically responsive to bone morphogenetic proteins. Recent results provide direct biochemical evidence that nodal signaling is mediated by SMAD2 and SMAD3, which also mediate signaling by TGF-betas and activins. Further evidence has shown that the extracellular protein Cripto or Cryptic is required for nodal signaling, making it distinct from activin or TGF-beta signaling.

The BMPs and GDFs together form a family of cysteine-knot cytokines sharing the characteristic fold of the TGF-beta superfamily. See, e.g., Rider et al. (2010) Biochem J., 429(1):1-12. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGF-beta superfamily ligands.

Growth and differentiation factor-8 (GDF8) is also known as myostatin. GDF8 is a negative regulator of skeletal muscle mass and is highly expressed in developing and adult skeletal muscle. The GDF8 null mutation in transgenic mice is characterized by a marked hypertrophy and hyperplasia of skeletal muscle. See, e.g., McPherron et al., Nature (1997) 387:83-90. Similar increases in skeletal muscle mass are evident in naturally occurring mutations of GDF8 in cattle and, strikingly, in humans. See, e.g., Ashmore et al. (1974) Growth, 38:501-507; Swatland and Kieffer, J. Anim. Sci. (1994) 38:752-757; McPherron and Lee, Proc. Natl. Acad. Sci. USA (1997) 94:12457-12461; Kambadur et al., Genome Res. (1997) 7:910-915; and Schuelke et al. (2004) N Engl J Med, 350:2682-8. Studies have also shown that muscle wasting associated with HIV-infection in humans is accompanied by increases in GDF8 protein expression. See, e.g., Gonzalez-Cadavid et al., PNAS (1998) 95:14938-43. In addition, GDF8 can modulate the production of muscle-specific enzymes (e.g., creatine kinase) and modulate myoblast cell proliferation. See, e.g., International Patent Application Publication No. WO 00/43781). The GDF8 propeptide can noncovalently bind to the mature GDF8 domain dimer, inactivating its biological activity. See, e.g., Miyazono et al. (1988) J. Biol. Chem., 263: 6407-6415; Wakefield et al. (1988) J. Biol. Chem., 263; 7646-7654; and Brown et al. (1990) Growth Factors, 3: 35-43. Other proteins which bind to GDF8 or structurally related proteins and inhibit their biological activity include follistatin, and potentially, follistatin-related proteins. See, e.g., Gamer et al. (1999) Dev. Biol., 208: 222-232.

GDF11, also known as BMP11, is a secreted protein that is expressed in the tail bud, limb bud, maxillary and mandibular arches, and dorsal root ganglia during mouse development. See, e.g., McPherron et al. (1999) Nat. Genet., 22: 260-264; and Nakashima et al. (1999) Mech. Dev., 80: 185-189. GDF11 plays a unique role in patterning both mesodermal and neural tissues. See, e.g., Gamer et al. (1999) Dev Biol., 208:222-32. GDF11 was shown to be a negative regulator of chondrogenesis and myogenesis in developing chick limb. See, e.g., Gamer et al. (2001) Dev Biol., 229:407-20. The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium. See, e.g., Wu et al. (2003) Neuron., 37:197-207. Hence, GDF11 may have in vitro and in vivo applications in the treatment of diseases such as muscle diseases and neurodegenerative diseases (e.g., amyotrophic lateral sclerosis).

BMP7, also called osteogenic protein-1 (OP-1), is well known to induce cartilage and bone formation. In addition, BMP7 regulates a wide array of physiological processes. For example, BMP7 may be the osteoinductive factor responsible for the phenomenon of epithelial osteogenesis. It is also found that BMP7 plays a role in calcium regulation and bone homeostasis. Like activin, BMP7 binds to type II receptors, ActRIIA and ActRIIB. However, BMP7 and activin recruit distinct type I receptors into heteromeric receptor complexes. The major BMP7 type I receptor observed was ALK2, while activin bound exclusively to ALK4 (ActRIIB). BMP7 and activin elicited distinct biological responses and activated different SMAD pathways. See, e.g., Macias-Silva et al. (1998) J Biol Chem. 273:25628-36.

Anti-Mullerian hormone (AMH), also known as Mullerian-inhibiting substance (MIS), is a TGF-beta family glycoprotein. One AMH-associated type II receptor has been identified and is designated as AMHRII, or alternatively MISRII. AMH induces regression of the Mullerian ducts in the human male embryo. AMH is expressed in reproductive age women and does not fluctuate with cycle or pregnancy, but was found to gradual decrease as both oocyte quantity and quality decrease, suggesting AMH could serve as a biomarker for ovarian physiology. See e.g. Zec et al., (2011) Biochemia Medica 21(3): 219-30.

Activin receptor-like kinase-1 (ALK1), the product of the ACVRL1 gene known alternatively as ACVRLK1, is a type I receptor whose expression is predominantly restricted to endothelial cells. See, e.g., OMIM entry 601284. ALK1 is activated by the binding of TGF-beta family ligands such as BMP9 and BMP10, and ALK1 signaling is critical in the regulation of both developmental and pathological blood vessel formation. ALK1 expression overlaps with sites of vasculogenesis and angiogenesis in early mouse development, and ALK1 knockout mice die around embryonic day 11.5 because of severe vascular abnormalities (see e.g., Cunha and Pietras (2011) Blood 117(26):6999-7006.) ALK1 expression has also been described in other cell types such as hepatic stellate cells and chondrocytes. Additionally, ALK1 along with activin receptor-like kinase-2 (ALK2) have been found to be important for BMP9-induced osteogenic signaling in mesenchymal stem cells. See e.g., Cunha and Pietras (2011) Blood 117(26):6999-7006.

ALK2, the product of the ACVR1 gene known alternatively as ActRIA or ACVRLK2, is a type I receptor that has been shown to bind activins and BMPs. ALK2 is critical for embryogenesis as ALK2 knockout mice die soon after gastrulation. See, e.g., Mishina et al. (1999) Dev Biol. 213: 314-326 and OMIM entry 102576. Constitutively active mutations in ALK2 are associated with fibrodysplasia ossificans progressiva (FOP). FOP is rare genetic disorder that causes fibrous tissue, including muscle, tendon and ligament, to be ossified spontaneously or when damaged. An arginine to histidine mutation in codon 206 of ALK2 is naturally occurring mutation associated with FOP in humans. This mutation induces BMP-specific signaling via ALK2 without the binding of ligand. See, e.g., Fukuda et al., (2009) J Biol Chem. 284(11):7149-7156 and Kaplan et al., (2011) Ann N.Y. Acad Sci. 1237: 5-10.

Activin receptor-like kinase-3 (ALK3), the product of the BMPR1A gene known alternatively as ACVRLK3, is a type I receptor mediating effects of multiple ligands in the BMP family. Unlike several type I receptors with ubiquitous tissue expression, ALK3 displays a restricted pattern of expression consistent with more specialized functionality. See, e.g., ten Dijke (1993) Oncogene, 8: 2879-2887 and OMIM entry 601299. ALK3 is generally recognized as a high affinity receptor for BMP2, BMP4, BMP7 and other members of the BMP family. BMP2 and BMP7 are potent stimulators of osteoblastic differentiation, and are now used clinically to induce bone formation in spine fusions and certain non-union fractures. ALK3 is regarded as a key receptor in mediating BMP2 and BMP4 signaling in osteoblasts. See, e.g., Lavery et al. (2008) J. Biol. Chem. 283: 20948-20958. A homozygous ALK3 knockout mouse dies early in embryogenesis (˜day 9.5), however, adult mice carrying a conditional disruption of ALK3 in osteoblasts have been recently reported to exhibit increased bone mass, although the newly formed bone showed evidence of disorganization. See, e.g., Kamiya (2008) J. Bone Miner. Res., 23:2007-2017; and Kamiya (2008) Development 135: 3801-3811. This finding is in startling contrast to the effectiveness of BMP2 and BMP7 (ligands for ALK3) as bone building agents in clinical use.

Activin receptor-like kinase-4 (ALK4), the product of the ACVR1B gene alternatively known as ACVRLK4, is a type I receptor that transduces signaling for a number of TGF-beta family ligands including activins, nodal and GDFs. ALK4 mutations are associated with pancreatic cancer and expression of dominant negative truncated ALK4 isoforms are highly expressed in human pituitary tumors. See, e.g., Tsuchida et al., (2008) Endocrine Journal 55(1):11-21 and OMIM entry 601300.

Activin receptor-like kinase-5 (ALK5), the product of the TGFBR1 gene, is widely expressed in most cell types. Several TGF-beta superfamily ligands, including TGF-betas, activin, and GDF-8, signal via ALK5 and activate downstream Smad 2 and Smad 3. Mice deficient in ALK5 exhibit severe defects in the vascular development of the yolk sac and placenta, lack circulating red blood cells, and die mid-gestation. It was found that these embryos had normal hematopoietic potential, but enhanced proliferation and improper migration of endothelial cells. Thus, ALK5-dependent signaling is important for angiogenesis, but not for the development of hematopoietic progenitor cells and functional hematopoiesis. See, e.g. Larsson et al., (2001) The EMBO Journal, 20(7): 1663-1673 and OMIM entry 190181. In endothelial cells, ALK5 acts cooperatively and opposite to ALK1 signaling. ALK5 inhibits cell migration and proliferation, notably the opposite effect of ALK1. See, e.g., Goumans et al. (2003) Mol Cell 12(4): 817-828. Additionally, ALK5 is believed to negatively regulate muscle growth. Knockdown of ALK5 in the muscle a mouse model of muscular dystrophy was found to decrease fibrosis and increase expression of genes associate with muscle growth. See, e.g. Kemaladewi et al., (2014) Mol Ther Nucleic Acids 3, e156.

Activin receptor-like kinase-6 (ALK6) is the product of the BMPR1B gene, whose deficiency is associated with chrondodysplasia and limb defects in both humans and mice. See, e.g., Demirhan et al., (2005) J Med Genet. 42:314-317. ALK6 is widely expressed throughout the developing skeleton, and is required for chondrogenesis in mice. See, e.g., Yi et al., (2000) Development 127:621-630 and OMIM entry 603248.

Activin receptor-like kinase-7 (ALK7) is the product of the ACVR1C gene. ALK7 null mice are viable, fertile, and display no skeletal or limb malformations. GDF3 signaling through ALK7 appears to play a role in insulin sensitivity and obesity. This is supported by results that Alk7 null mice show reduced fat accumulation and resistance to diet-induced obesity. See, e.g., Andersson et al., (2008) PNAS 105(20): 7252-7256. ALK7-mediated Nodal signaling has been implicated to have both tumor promoting and tumor suppressing effects in a variety of different cancer cell lines. See, e.g., De Silva et al., (2012) Frontiers in Endocrinology 3:59 and OMIM entry 608981.

As used herein the term “ActRII” refers to the family of type II activin receptors. This family includes both the activin receptor type IIA (ActRIIA), encoded by the ACVR2A gene, and the activin receptor type IIB (ActRIIB), encoded by the ACVR2B gene. ActRII receptors are TGF-beta superfamily type II receptors that bind a variety of TGF-beta superfamily ligands including activins, GDF8 (myostatin), GDF11, and a subset of BMPs, notably BMP6 and BMP7. ActRII receptors are implicated in a variety of biological disorders including muscle and neuromuscular disorders (e.g., muscular dystrophy, amyotrophic lateral sclerosis (ALS), and muscle atrophy), undesired bone/cartilage growth, adipose tissue disorders (e.g., obesity), metabolic disorders (e.g., type 2 diabetes), and neurodegenerative disorders. See, e.g., Tsuchida et al., (2008) Endocrine Journal 55(1):11-21, Knopf et al., U.S. Pat. No. 8,252,900, and OMIM entries 102581 and 602730.

Transforming growth factor beta receptor II (TGFBRII), encoded by the TGFBR2 gene, is a type II receptor that is known to bind TGF-beta ligands and activate downstream Smad 2 and Smad 3 effectors. See, e.g., Hinck (2012) FEBS Letters 586: 1860-1870 and OMIM entry 190182. TGF-beta signaling through TGFBRII is critical in T-cell proliferation, maintenance of T regulatory cells and proliferation of precartilaginous stem cells. See, e.g., Li et al., (2006) Immunity 25(3): 455-471 and Cheng et al., Int. J. Mol. Sci. 2014, 15, 12665-12676.

Bone morphogenetic protein receptor II (BMPRII), encoded by the BMPR2 gene, is a type II receptor that is thought to bind certain BMP ligands. In some instances, efficient ligand binding to BMPRII is dependent on the presence of the appropriate TGFBR type I receptors. See, e.g., Rosenzweig et al., (1995) PNAS 92:7632-7636. Mutations in BMPRII are associated pulmonary hypertension in humans. See OMIM entry 600799.

Müllerian-inhibiting substance receptor II (MISRII), the product of the AMHR2 gene known alternatively as anti-Müllerian hormone type II receptor, is a type II TGF-beta receptor. MISRII binds the MIS ligand, but requires the presence of an appropriate type I receptor, such as ALK3 or ALK6, for signal transduction. See, e.g., Hinck (2012) FEBS Letters 586:1860-1870 and OMIM entry 600956. MISRII is involved in sex differentiation in humans and is required for Müllerian regression in the human male. AMH is expressed in reproductive age women and does not fluctuate with cycle or pregnancy, but was found to gradual decrease as both oocyte quantity and quality decrease, suggesting AMH could serve as a biomarker of ovarian physiology. See, e.g., Zec et al., (2011) Biochemia Medica 21(3): 219-30 and OMIM entry 600956.

In certain aspects, the present disclosure relates to the use of a) heteromultimer complexes comprising an extracellular domain of a TGFβ superfamily type I receptor polypeptide (e.g., ALK1, ALK2, ALK3, ALK4, ALK5, ALK6, and ALK7) and an extracellular domain of a TGFβ superfamily type II receptor polypeptide (e.g., ActRIIA, ActRIIB, TGFBRII, BMPRII, and MISRII) b) heteromultimer complexes comprising an extracellular domain of at least two TGFβ superfamily type I receptor polypeptide (e.g., ALK1, ALK2, ALK3, ALK4, ALK5, ALK6, and ALK7), and heteromultimer complexes comprising an extracellular domain of at least two TGFβ superfamily type II receptor polypeptide (e.g., ActRIIA, ActRIIB, TGFBRII, BMPRII, and MISRII), preferably soluble heteromultimer complexes, to antagonize intracellular signaling transduction (e.g., Smad 2/3 and/or Smad 1/5/8 signaling) initiated by one or more TGFβ superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, Müllerian-inhibiting substance (MIS), and Lefty). As described herein, such antagonist heteromultimer complexes may be useful in the treatment or prevention of various disorders/conditions associated with, e.g., muscle loss, insufficient muscle growth, neurodegeneration, bone loss, reduced bone density and/or mineralization, insufficient bone growth, metabolic disorders such as obesity and red blood cell disorders such as anemia.

In particular, the data of the present disclosure demonstrates that heteromultimer complexes comprising an extracellular domain of a TGFβ superfamily type I receptor polypeptide and an extracellular domain of a TGFβ superfamily type II receptor polypeptide have different ligand binding specificities/profiles in comparison to their corresponding homomultimer complexes.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The terms “heteromer” or “heteromultimer” is a complex comprising at least a first polypeptide and a second polypeptide, wherein the second polypeptide differs in amino acid sequence from the first polypeptide by at least one amino acid residue. The heteromer can comprise a “heterodimer” formed by the first and second polypeptide or can form higher order structures where polypeptides in addition to the first and second polypeptide are present. Exemplary structures for the heteromultimer include heterodimers, heterotrimers, heterotetramers and further oligomeric structures. Heterodimers are designated herein as X:Y or equivalently as X-Y, where X represents a first polypeptide and Y represents a second polypeptide. Higher-order heteromers and oligomeric structures are designated herein in a corresponding manner. In certain embodiments a heteromultimer is recombinant (e.g., one or more polypeptide components may be a recombinant protein), isolated and/or purified.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

As used herein, unless otherwise stated, “does not substantially bind to X” is intended to mean that an agent has a K_{D} that is greater than about 10⁻⁷, 10⁻⁶, 10⁻⁵, 10⁻⁴, or greater (e.g., no detectable binding by the assay used to determine the K_{D}) for “X”, wherein “X” is a specified agent such as protein or nucleic acid.

The terms “about” and “approximately” as used in connection with a numerical value throughout the specification and the claims denotes an interval of accuracy, familiar and acceptable to a person skilled in the art. In general, such interval of accuracy is +10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. TGF-beta Superfamily Type I Receptor and Type II Receptor Complexes

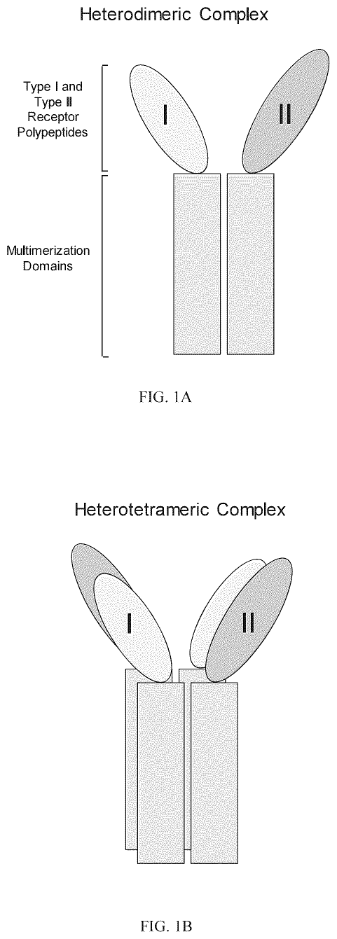

In certain aspects, the present disclosure relates to heteromultimer complexes comprising one or more TGF-beta superfamily type I receptor polypeptides (e.g., ALK1, ALK2, ALK3, ALK4, ALK5, ALK6, and ALK7 proteins from humans or other species such as those described herein, e.g., SEQ ID NOs: 14, 15, 124, 126, 171, 172, 413, 414, 463, 464, 18, 19, 136, 138, 173, 174, 421, 422, 465, 466, 22, 23, 115, 117, 175, 176, 407, 408, 467, 468, 26, 27, 83, 84, 104, 106, 177, 178, 403, 404, 469, 470, 30, 31, 87, 88, 139, 141, 179, 180, 423, 424, 471, 472, 34, 35, 91, 92, 142, 144, 181, 182, 425, 426, 473, 474, 38, 39, 301, 302, 305, 306, 309, 310, 313, 112, 114, 183, 184, 405, 406, 475, and 476) and one or more TGF-beta superfamily type II receptor polypeptides (e.g., ActRIIA, ActRIIB, TGFBRII, BMPRII, and MISRII proteins from humans or other species such as those described herein, e.g., SEQ ID NOs: 9, 10, 11, 118, 120, 151, 152, 409, 410, 451, 452, 1, 2, 3, 4, 5, 6, 100, 102, 153, 154, 401, 402, 453, 454, 46, 47, 71, 72, 121, 123, 155, 156, 411, 412, 455, 456, 50, 51, 75, 76, 79, 80, 133, 135, 161, 162, 419, 420, 457, 458, 42, 43, 67, 68, 127, 129, 130, 132, 157, 158, 159, 160, 415, 416, 417, 418, 459, 460, 461, and 462); heteromultimer complexes comprising at least two different TGF-beta superfamily type I receptor polypeptides (e.g., ALK1, ALK2, ALK3, ALK4, ALK5, ALK6, and ALK7 proteins from humans or other species such as those described herein, e.g., SEQ ID NOs: 14, 15, 124, 126, 171, 172, 413, 414, 463, 464, 18, 19, 136, 138, 173, 174, 421, 422, 465, 466, 22, 23, 115, 117, 175, 176, 407, 408, 467, 468, 26, 27, 83, 84, 104, 106, 177, 178, 403, 404, 469, 470, 30, 31, 87, 88, 139, 141, 179, 180, 423, 424, 471, 472, 34, 35, 91, 92, 142, 144, 181, 182, 425, 426, 473, 474, 38, 39, 301, 302, 305, 306, 309, 310, 313, 112, 114, 183, 184, 405, 406, 475, and 476); and heteromultimer complexes comprising at least two different TGF-beta superfamily type II receptor polypeptides (e.g., ActRIIA, ActRIIB, TGFBRII, BMPRII, and MISRII proteins from humans or other species such as those described herein, e.g., SEQ ID NOs: 9, 10, 11, 118, 120, 151, 152, 409, 410, 451, 452, 1, 2, 3, 4, 5, 6, 100, 102, 153, 154, 401, 402, 453, 454, 46, 47, 71, 72, 121, 123, 155, 156, 411, 412, 455, 456, 50, 51, 75, 76, 79, 80, 133, 135, 161, 162, 419, 420, 457, 458, 42, 43, 67, 68, 127, 129, 130, 132, 157, 158, 159, 160, 415, 416, 417, 418, 459, 460, 461, and 462), which are generally referred to herein as “heteromultimer complexes” or “heteromultimers”. Preferably, heteromultimers are soluble, e.g., a heteromultimer comprises a soluble portion of at least one TGFβ superfamily type I receptor polypeptide and a soluble portion (domain) of at least one TGFβ superfamily type II receptor polypeptide. In general, the extracellular domains of TGFβ superfamily type I and type II receptors correspond to a soluble portion of the type I and type II receptor. Therefore, in some embodiments, heteromultimers of the disclosure comprise an extracellular domain of a TGFβ superfamily type I receptor polypeptide (e.g., one or more ALK1, ALK2, ALK3, ALK4, ALK5, ALK6, and/or ALK7 receptor extracellular domains) and/or an extracellular domain of a TGFβ superfamily type II receptor polypeptide (e.g., one or more ActRIIA, ActRIIB, TGFBRII, BMPRII, and/or MISRII receptor extracellular domains). Exemplary extracellular domains of ALK1, ALK2, ALK3, ALK4, ALK5, ALK6, ALK7, ActRIIA, ActRIIB, TGFBRII, BMPRII, and MISRII are disclosed herein and such sequences, as well as fragments, functional variants, and modified forms thereof, may be used in accordance with the inventions of the present disclosure (e.g., heteromultimers compositions and uses thereof). Heteromultimers of the disclosure include, e.g., heterodimers, heterotrimers, heterotetramers, and higher order oligomeric structures. See, e.g., FIGS. 1, 2, and 15-17. In certain preferred embodiments, heteromultimers of the disclosure are heterodimers.

A defining structural motif known as a three-finger toxin fold is important for ligand binding by type I and type II receptors and is formed by 10, 12, or 14 conserved cysteine residues located at varying positions within the extracellular domain of each monomeric receptor. See, e.g., Greenwald et al. (1999) Nat Struct Biol 6:18-22; Hinck (2012) FEBS Lett 586:1860-1870. The core ligand-binding domains of TGFβ superfamily receptors, as demarcated by the outermost of these conserved cysteines, corresponds to positions 29-109 of SEQ ID NO: 1 (ActRIIB precursor); positions 30-110 of SEQ ID NO: 9 (ActRIIA precursor); positions 34-95 of SEQ ID NO: 14 (ALK1 precursor); positions 35-99 of SEQ ID NO: 18 (ALK2 precursor); positions 61-130 of SEQ ID NO: 22 (ALK3 precursor); positions 34-101 of SEQ ID NOs: 26 and 83 (ALK4 precursors); positions 36-106 of SEQ ID NOs: 30 and 87 (ALK5 precursors); positions 32-102 of SEQ ID NO: 34 (ALK6 isoform B precursor); positions 28-92 of SEQ ID NOs: 38, 305, and 309 (ALK7 precursors); positions 51-143 of SEQ ID NO: 42 (TGFBRII isoform B precursor); positions 34-123 of SEQ ID NO: 46 and 71 (BMPRII precursors); positions 24-116 of SEQ ID NO: 50, 75, and 79 (MISRII precursors); positions 44-168 of SEQ ID NO: 67 (TGFBRII isoform A precursor); and positions 62-132 of SEQ ID NO: 91 (ALK6 isoform A precursor). The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 residues on either terminus without necessarily altering ligand binding. Exemplary extracellular domains for N-terminal and/or C-terminal truncation include SEQ ID NOs: 2, 3, 5, 6, 10, 11, 15, 19, 23, 27, 31, 35, 39, 43, 47, 51, 68, 72, 76, 80, 84, 88, 92, 302, 306, 310, and 313.

In preferred embodiments, heteromultimers of the disclosure bind to and/or inhibit (antagonize) activity of one or more TGF-beta superfamily ligands including, but not limited to, BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty. In particular, heteromultimer complexes of the disclosure may be used to antagonize signaling transduction (e.g., Smad 2/3 and/or Smad 1/5/8 signaling) initiated by one or more TGFβ superfamily ligands, which may be determined, for example, using a cell-based assay such as those described herein. As described herein, such antagonist heteromultimer complexes may be useful in the treatment or prevention of various disorders/conditions associated with, e.g., muscle loss, insufficient muscle growth, neurodegeneration, bone loss, reduced bone density and/or mineralization, insufficient bone growth, and/or obesity. In some embodiments, heteromultimer complexes of the disclosure have different ligand binding specificities/profiles in comparison to their corresponding homomultimer complex (e.g., an ALK4:ActRIIB heterodimer vs. a corresponding ActRIIB or ALK4 homodimer).

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication Nos. WO 2006/012627, WO 2008/097541, and Wo 2010/151426, which are incorporated herein by reference in their entirety. Numbering of amino acids for all ActRIIB-related polypeptides described herein is based on the numbering of the human ActRIIB precursor protein sequence provided below (SEQ ID NO: 1), unless specifically designated otherwise.

The human ActRIIB precursor protein sequence is as follows:

| (SEQ ID NO: 1) | |
|---|---|
| 1 | MTAPWVALAL LWGSLCAGSG RGEAETRECI YYNANWELER TNQSGLERCE |
| 51 | GEQDKRLHCY ASWRNSSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated with a single underline; the extracellular domain is indicated in bold font; and the potential, endogenous N-linked glycosylation sites are indicated with a double underline.

The processed (mature) extracellular ActRIIB polypeptide sequence is as follows:

(SEQ ID NO: 2)
GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAPT.

In some embodiments, the protein may be produced with an “SGR . . . ” sequence at the N-terminus. The C-terminal “tail” of the extracellular domain is indicated by a single underline. The sequence with the “tail” deleted (a A15 sequence) is as follows:

(SEQ ID NO: 3)
GRGEAETRECIYYNANWELERTNQSGLERCEGEQDKRLHCYASWRNSSGT
IELVKKGCWLDDFNCYDRQECVATEENPQVYFCCCEGNFCNERFTHLPE
A.

A form of ActRIIB with an alanine at position 64 of SEQ ID NO: 1 (A64) is also reported in the literature. See, e.g., Hilden et al. (1994) Blood, 83(8): 2163-2170. Applicants have ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure. The form of ActRIIB with an alanine at position 64 is as follows:

| (SEQ ID NO: 4) | |
|---|---|
| 1 | MTAPWVALAL LWGSLCAGSG RGEAETRECI YYNANWELER TNQSGLERCE |
| 51 | GEQDKRLHCY ASWANSSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated by single underline and the extracellular domain is indicated by bold font.

The processed (mature) extracellular ActRIIB polypeptide sequence of the alternative A64 form is as follows:

(SEQ ID NO: 5)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA
GGPEVTYEPPPTAPT

In some embodiments, the protein may be produced with an “SGR . . . ” sequence at the N-terminus. The C-terminal “tail” of the extracellular domain is indicated by single underline. The sequence with the “tail” deleted (a A15 sequence) is as follows:

(SEQ ID NO: 6)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA

A nucleic acid sequence encoding the human ActRIIB precursor protein is shown below (SEQ ID NO: 7), representing nucleotides 25-1560 of Genbank Reference Sequence NM_001106.3, which encode amino acids 1-513 of the ActRIIB precursor. The sequence as shown provides an arginine at position 64 and may be modified to provide an alanine instead. The signal sequence is underlined.

| (SEQ ID NO: 7) | |
|---|---|
| 1 | ATGACGGCGC CCTGGGTGGC CCTCGCCCTC CTCTGGGGAT CGCTGTGCGC |
| 51 | CGGCTCTGGG CGTGGGGAGG CTGAGACACG GGAGTGCATC TACTACAACG |
| 101 | CCAACTGGGA GCTGGAGCGC ACCAACCAGA GCGGCCTGGA GCGCTGCGAA |
| 151 | GGCGAGCAGG ACAAGCGGCT GCACTGCTAC GCCTCCTGGC GCAACAGCTC |
| 201 | TGGCACCATC GAGCTCGTGA AGAAGGGCTG CTGGCTAGAT GACTTCAACT |
| 251 | GCTACGATAG GCAGGAGTGT GTGGCCACTG AGGAGAACCC CCAGGTGTAC |
| 301 | TTCTGCTGCT GTGAAGGCAA CTTCTGCAAC GAACGCTTCA CTCATTTGCC |
| 351 | AGAGGCTGGG GGCCCGGAAG TCACGTACGA GCCACCCCCG ACAGCCCCCA |
| 401 | CCCTGCTCAC GGTGCTGGCC TACTCACTGC TGCCCATCGG GGGCCTTTCC |
| 451 | CTCATCGTCC TGCTGGCCTT TTGGATGTAC CGGCATCGCA AGCCCCCCTA |
| 501 | CGGTCATGTG GACATCCATG AGGACCCTGG GCCTCCACCA CCATCCCCTC |
| 551 | TGGTGGGCCT GAAGCCACTG CAGCTGCTGG AGATCAAGGC TCGGGGGCGC |
| 601 | TTTGGCTGTG TCTGGAAGGC CCAGCTCATG AATGACTTTG TAGCTGTCAA |
| 651 | GATCTTCCCA CTCCAGGACA AGCAGTCGTG GCAGAGTGAA CGGGAGATCT |
| 701 | TCAGCACACC TGGCATGAAG CACGAGAACC TGCTACAGTT CATTGCTGCC |
| 751 | GAGAAGCGAG GCTCCAACCT CGAAGTAGAG CTGTGGCTCA TCACGGCCTT |
| 801 | CCATGACAAG GGCTCCCTCA CGGATTACCT CAAGGGGAAC ATCATCACAT |
| 851 | GGAACGAACT GTGTCATGTA GCAGAGACGA TGTCACGAGG CCTCTCATAC |
| 901 | CTGCATGAGG ATGTGCCCTG GTGCCGTGGC GAGGGCCACA AGCCGTCTAT |
| 951 | TGCCCACAGG GACTTTAAAA GTAAGAATGT ATTGCTGAAG AGCGACCTCA |
| 1001 | CAGCCGTGCT GGCTGACTTT GGCTTGGCTG TTCGATTTGA GCCAGGGAAA |
| 1051 | CCTCCAGGGG ACACCCACGG ACAGGTAGGC ACGAGACGGT ACATGGCTCC |
| 1101 | TGAGGTGCTC GAGGGAGCCA TCAACTTCCA GAGAGATGCC TTCCTGCGCA |
| 1151 | TTGACATGTA TGCCATGGGG TTGGTGCTGT GGGAGCTTGT GTCTCGCTGC |
| 1201 | AAGGCTGCAG ACGGACCCGT GGATGAGTAC ATGCTGCCCT TTGAGGAAGA |
| 1251 | GATTGGCCAG CACCCTTCGT TGGAGGAGCT GCAGGAGGTG GTGGTGCACA |
| 1301 | AGAAGATGAG GCCCACCATT AAAGATCACT GGTTGAAACA CCCGGGCCTG |
| 1351 | GCCCAGCTTT GTGTGACCAT CGAGGAGTGC TGGGACCATG ATGCAGAGGC |
| 1401 | TCGCTTGTCC GCGGGCTGTG TGGAGGAGCG GGTGTCCCTG ATTCGGAGGT |
| 1451 | CGGTCAACGG CACTACCTCG GACTGTCTCG TTTCCCTGGT GACCTCTGTC |
| 1501 | ACCAATGTGG ACCTGCCCCC TAAAGAGTCA AGCATC |

A nucleic acid sequence encoding processed extracellular human ActRIIB polypeptide is as follows (SEQ ID NO: 8). The sequence as shown provides an arginine at position 64, and may be modified to provide an alanine instead.

| (SEQ ID NO: 8) | |
|---|---|
| 1 | GGGCGTGGGG AGGCTGAGAC ACGGGAGTGC ATCTACTACA ACGCCAACTG |
| 51 | GGAGCTGGAG CGCACCAACC AGAGCGGCCT GGAGCGCTGC GAAGGCGAGC |
| 101 | AGGACAAGCG GCTGCACTGC TACGCCTCCT GGCGCAACAG CTCTGGCACC |
| 151 | ATCGAGCTCG TGAAGAAGGG CTGCTGGCTA GATGACTTCA ACTGCTACGA |
| 201 | TAGGCAGGAG TGTGTGGCCA CTGAGGAGAA CCCCCAGGTG TACTTCTGCT |
| 251 | GCTGTGAAGG CAACTTCTGC AACGAACGCT TCACTCATTT GCCAGAGGCT |
| 301 | GGGGGCCCGG AAGTCACGTA CGAGCCACCC CCGACAGCCC CCACC |

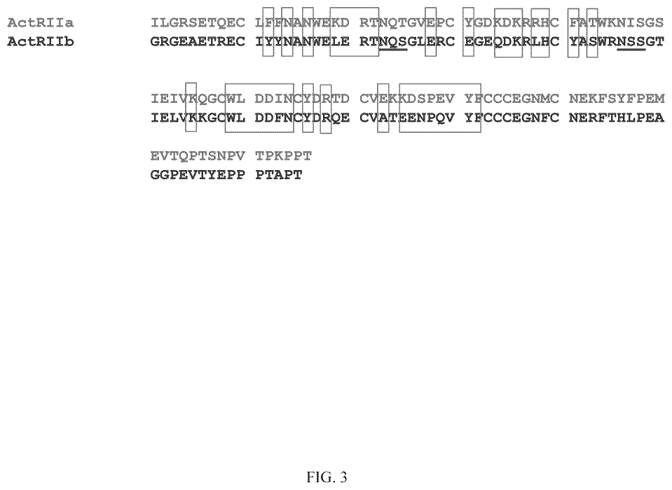

An alignment of the amino acid sequences of human ActRIIB extracellular domain and human ActRIIA extracellular domain are illustrated in FIG. 3. This alignment indicates amino acid residues within both receptors that are believed to directly contact ActRII ligands. For example, the composite ActRII structures indicated that the ActRIIB-ligand binding pocket is defined, in part, by residues Y31, N33, N35, L38 through T41, E47, E50, Q53 through K55, L57, H58, Y60, S62, K74, W78 through N83, Y85, R87, A92, and E94 through F101. At these positions, it is expected that conservative mutations will be tolerated.

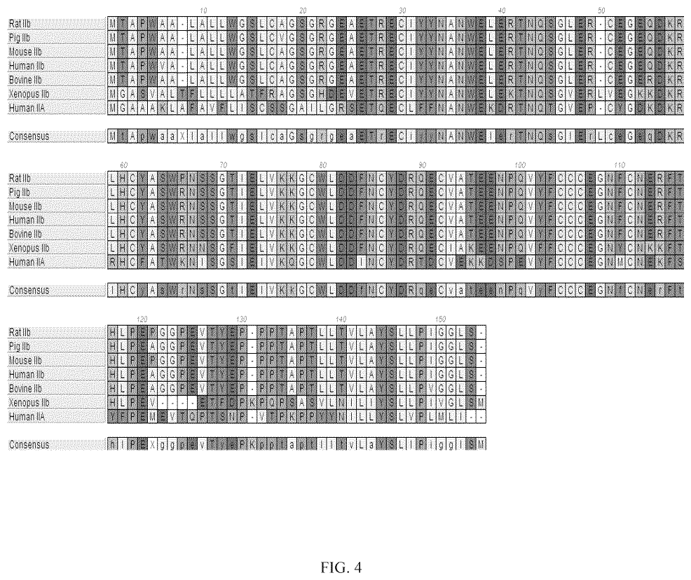

In addition, ActRIIB is well-conserved among vertebrates, with large stretches of the extracellular domain completely conserved. For example, FIG. 4 depicts a multi-sequence alignment of a human ActRIIB extracellular domain compared to various ActRIIB orthologs. Many of the ligands that bind to ActRIIB are also highly conserved. Accordingly, from these alignments, it is possible to predict key amino acid positions within the ligand-binding domain that are important for normal ActRIIB-ligand binding activities as well as to predict amino acid positions that are likely to be tolerant of substitution without significantly altering normal ActRIIB-ligand binding activities. Therefore, an active, human ActRIIB variant polypeptide useful in accordance with the presently disclosed methods may include one or more amino acids at corresponding positions from the sequence of another vertebrate ActRIIB, or may include a residue that is similar to that in the human or other vertebrate sequences. Without meaning to be limiting, the following examples illustrate this approach to defining an active ActRIIB variant. L46 in the human extracellular domain (SEQ ID NO: 2) is a valine in Xenopus ActRIIB (SEQ ID NO: 506), and so this position may be altered, and optionally may be altered to another hydrophobic residue, such as V, I or F, or a non-polar residue such as A. E52 in the human extracellular domain is a K in Xenopus, indicating that this site may be tolerant of a wide variety of changes, including polar residues, such as E, D, K, R, H, S, T, P, G, Y and probably A. T93 in the human extracellular domain is a K in Xenopus, indicating that a wide structural variation is tolerated at this position, with polar residues favored, such as S, K, R, E, D, H, G, P, G and Y. F108 in the human extracellular domain is a Y in Xenopus, and therefore Y or other hydrophobic group, such as I, V or L should be tolerated. E111 in the human extracellular domain is K in Xenopus, indicating that charged residues will be tolerated at this position, including D, R, K and H, as well as Q and N. R112 in the human extracellular domain is K in Xenopus, indicating that basic residues are tolerated at this position, including R and H. A at position 119 in the human extracellular domain is relatively poorly conserved, and appears as P in rodents and V in Xenopus, thus essentially any amino acid should be tolerated at this position.

Moreover, ActRII proteins have been characterized in the art in terms of structural and functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide amply guidance for how to generate ActRIIB variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIB, as demarcated by the outermost of these conserved cysteines, corresponds to positions 29-109 of SEQ ID NO: 1 (ActRIIB precursor). Thus, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, or 28 residues at the N-terminus and/or by about 1, 2, 3, 4, 5, 6, 7, 8, 9, 10, 11, 12, 13, 14, 15, 16, 17, 18, 19, 20, 21, 22, 23, 24, or 25 residues a the C-terminus without necessarily altering ligand binding. Exemplary ActRIIB extracellular domains for N-terminal and/or C-terminal truncation include SEQ ID NOs: 2, 3, 5, and 6.

Attisano et al. showed that a deletion of the proline knot at the C-terminus of the extracellular domain of ActRIIB reduced the affinity of the receptor for activin. An ActRIIB-Fc fusion protein containing amino acids 20-119 of present SEQ ID NO: 1, “ActRIIB(20-119)-Fc”, has reduced binding to GDF11 and activin relative to an ActRIIB(20-134)-Fc, which includes the proline knot region and the complete juxtamembrane domain (see, e.g., U.S. Pat. No. 7,842,663). However, an ActRIIB(20-129)-Fc protein retains similar, but somewhat reduced activity, relative to the wild-type, even though the proline knot region is disrupted.

Thus, ActRIIB extracellular domains that stop at amino acid 134, 133, 132, 131, 130 and 129 (with respect to SEQ ID NO: 1) are all expected to be active, but constructs stopping at 134 or 133 may be most active. Similarly, mutations at any of residues 129-134 (with respect to SEQ ID NO: 1) are not expected to alter ligand-binding affinity by large margins. In support of this, it is known in the art that mutations of P129 and P130 (with respect to SEQ ID NO: 1) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO: 1) is poorly conserved and so is readily altered or truncated. ActRIIB polypeptides ending at 128 (with respect to SEQ ID NO: 1) or later should retain ligand-binding activity. ActRIIB polypeptides ending at or between 119 and 127 (e.g., 119, 120, 121, 122, 123, 124, 125, 126, or 127), with respect to SEQ ID NO: 1, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 1) will retain ligand-binding activity. Amino acid 29 represents the initial cysteine. An alanine-to-asparagine mutation at position 24 (with respect to SEQ ID NO: 1) introduces an N-linked glycosylation sequence without substantially affecting ligand binding [U.S. Pat. No. 7,842,663]. This confirms that mutations in the region between the signal cleavage peptide and the cysteine cross-linked region, corresponding to amino acids 20-29, are well tolerated. In particular, ActRIIB polypeptides beginning at position 20, 21, 22, 23, and 24 (with respect to SEQ ID NO: 1) should retain general ligand-biding activity, and ActRIIB polypeptides beginning at positions 25, 26, 27, 28, and 29 (with respect to SEQ ID NO: 1) are also expected to retain ligand-biding activity. It has been demonstrated, e.g., U.S. Pat. No. 7,842,663, that, surprisingly, an ActRIIB construct beginning at 22, 23, 24, or 25 will have the most activity.

Taken together, a general formula for an active portion (e.g., ligand-binding portion) of ActRIIB comprises amino acids 29-109 of SEQ ID NO: 1. Therefore ActRIIB polypeptides may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to any one of amino acids 20-29 (e.g., beginning at any one of amino acids 20, 21, 22, 23, 24, 25, 26, 27, 28, or 29) of SEQ ID NO: 1 and ending at a position corresponding to any one amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. Other examples include polypeptides that begin at a position from 20-29 (e.g., any one of positions 20, 21, 22, 23, 24, 25, 26, 27, 28, or 29) or 21-29 (e.g., any one of positions 21, 22, 23, 24, 25, 26, 27, 28, or 29) of SEQ ID NO: 1 and end at a position from 119-134 (e.g., any one of positions 119, 120, 121, 122, 123, 124, 125, 126, 127, 128, 129, 130, 131, 132, 133, or 134), 119-133 (e.g., any one of positions 119, 120, 121, 122, 123, 124, 125, 126, 127, 128, 129, 130, 131, 132, or 133), 129-134 (e.g., any one of positions 129, 130, 131, 132, 133, or 134), or 129-133 (e.g., any one of positions 129, 130, 131, 132, or 133) of SEQ ID NO: 1. Other examples include constructs that begin at a position from 20-24 (e.g., any one of positions 20, 21, 22, 23, or 24), 21-24 (e.g., any one of positions 21, 22, 23, or 24), or 22-25 (e.g., any one of positions 22, 22, 23, or 25) of SEQ ID NO: 1 and end at a position from 109-134 (e.g., any one of positions 109, 110, 111, 112, 113, 114, 115, 116, 117, 118, 119, 120, 121, 122, 123, 124, 125, 126, 127, 128, 129, 130, 131, 132, 133, or 134), 119-134 (e.g., any one of positions 119, 120, 121, 122, 123, 124, 125, 126, 127, 128, 129, 130, 131, 132, 133, or 134) or 129-134 (e.g., any one of positions 129, 130, 131, 132, 133, or 134) of SEQ ID NO: 1. Variants within these ranges are also contemplated, particularly those having at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1.

The variations described herein may be combined in various ways. In some embodiments, ActRIIB variants comprise no more than 1, 2, 5, 6, 7, 8, 9, 10 or 15 conservative amino acid changes in the ligand-binding pocket, and zero, one, or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 1). An asparagine-to-alanine alteration at position 65 (N65A) actually improves ligand binding in the A64 background, and is thus expected to have no detrimental effect on ligand binding in the R64 background [U.S. Pat. No. 7,842,663]. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64 [U.S. Pat. No. 7,842,663]. Additionally, the results of the mutagenesis program described in the art indicate that there are amino acid positions in ActRIIB that are often beneficial to conserve. With respect to SEQ ID NO: 1, these include position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, the disclosure provides a framework of amino acids that may be conserved in ActRIIB polypeptides. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 1.

In certain embodiments, the disclosure relates to heteromultimers that comprise at least one ActRIIB polypeptide, which includes fragments, functional variants, and modified forms thereof. Preferably, ActRIIB polypeptides for use in accordance with the disclosure are soluble (e.g., an extracellular domain of ActRIIB). In other preferred embodiments, ActRIIB polypeptides for use in accordance with the disclosure bind to one or more TGF-beta superfamily ligands. Therefore, in some embodiments, ActRIIB polypeptides for use in accordance with the disclosure inhibit (antagonize) activity (e.g., inhibition of Smad signaling) of one or more TGF-beta superfamily ligands. In some embodiments, heteromultimers of the disclosure comprise at least one ActRIIB polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to amino acids 20-29 (e.g., beginning at any one of amino acids 20, 21, 22, 23, 24, 25, 26, 27, 28, or 29) of SEQ ID NO: 1 and ending at a position corresponding to amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. In certain preferred embodiments, heteromultimers of the disclosure comprise at least one ActRIIB polypeptide that comprises, consists, or consists essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1 In other preferred embodiments, heteromultimers of the disclosure comprise at least one ActRIIB polypeptide that comprises, consists, or consists essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1 In some embodiments, heteromultimers of the disclosure comprise at least one ActRIIB polypeptide that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 100, 102, 153, 154, 401, 402, 453, and 454. In certain embodiments, heteromultimers of the disclosure comprise at least one ActRIIB polypeptide wherein the amino acid position corresponding to L79 of SEQ ID NO: 1 is not an acidic amino acid (i.e., is not a naturally occurring D or E amino acid residue or artificial acidic amino acid).

In certain embodiments, the present disclosure relates to a protein complex comprising an ActRIIA polypeptide. As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the present disclosure as well as in International Patent Application Publication No. WO 2006/012627, which is incorporated herein by reference in its entirety. Numbering of amino acids for all ActRIIA-related polypeptides described herein is based on the numbering of the human ActRIIA precursor protein sequence provided below (SEQ ID NO: 9), unless specifically designated otherwise.

The human ActRIIA precursor protein sequence is as follows:

| (SEQ ID NO: 9) | |
|---|---|
| 1 | MGAAAKLAFA VFLISCSSGA ILGRSETQEC LFFNANWEKD RTNQTGVEPC |
| 51 | YGDKDKRRHC FATWKNISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 151 | AGIVICAFWV YRHHKMAYPP VLVPTQDPGP PPPSPLLGLK PLQLLEVKAR |
| 201 | GRFGCVWKAQ LLNEYVAVKI FPIQDKQSWQ NEYEVYSLPG MKHENILQFI |
| 251 | GAEKRGTSVD VDLWLITAFH EKGSLSDFLK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFQRD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLTNIIT TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

The processed extracellular human ActRIIA polypeptide sequence is as follows:

(SEQ ID NO: 10)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPP

The C-terminal “tail” of the extracellular domain is indicated by a single underline. The sequence with the “tail” deleted (a A15 sequence) is as follows:

(SEQ ID NO: 11)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM

A nucleic acid sequence encoding the human ActRIIA precursor protein is shown below (SEQ ID NO: 12), corresponding to nucleotides 159-1700 of Genbank Reference Sequence NM_001616.4. The signal sequence is underlined.

| (SEQ ID NO: 12) | |
|---|---|
| 1 | ATGGGAGCTG CTGCAAAGTT GGCGTTTGCC GTCTTTCTTA TCTCCTGTTC |
| 51 | TTCAGGTGCT ATACTTGGTA GATCAGAAAC TCAGGAGTGT CTTTTCTTTA |
| 101 | ATGCTAATTG GGAAAAAGAC AGAACCAATC AAACTGGTGT TGAACCGTGT |
| 151 | TATGGTGACA AAGATAAACG GCGGCATTGT TTTGCTACCT GGAAGAATAT |
| 201 | TTCTGGTTCC ATTGAAATAG TGAAACAAGG TTGTTGGCTG GATGATATCA |
| 251 | ACTGCTATGA CAGGACTGAT TGTGTAGAAA AAAAAGACAG CCCTGAAGTA |
| 301 | TATTTTTGTT GCTGTGAGGG CAATATGTGT AATGAAAAGT TTTCTTATTT |
| 351 | TCCGGAGATG GAAGTCACAC AGCCCACTTC AAATCCAGTT ACACCTAAGC |
| 401 | CACCCTATTA CAACATCCTG CTCTATTCCT TGGTGCCACT TATGTTAATT |
| 451 | GCGGGGATTG TCATTTGTGC ATTTTGGGTG TACAGGCATC ACAAGATGGC |
| 501 | CTACCCTCCT GTACTTGTTC CAACTCAAGA CCCAGGACCA CCCCCACCTT |
| 551 | CTCCATTACT AGGTTTGAAA CCACTGCAGT TATTAGAAGT GAAAGCAAGG |
| 601 | GGAAGATTTG GTTGTGTCTG GAAAGCCCAG TTGCTTAACG AATATGTGGC |
| 651 | TGTCAAAATA TTTCCAATAC AGGACAAACA GTCATGGCAA AATGAATACG |
| 701 | AAGTCTACAG TTTGCCTGGA ATGAAGCATG AGAACATATT ACAGTTCATT |
| 751 | GGTGCAGAAA AACGAGGCAC CAGTGTTGAT GTGGATCTTT GGCTGATCAC |
| 801 | AGCATTTCAT GAAAAGGGTT CACTATCAGA CTTTCTTAAG GCTAATGTGG |
| 851 | TCTCTTGGAA TGAACTGTGT CATATTGCAG AAACCATGGC TAGAGGATTG |
| 901 | GCATATTTAC ATGAGGATAT ACCTGGCCTA AAAGATGGCC ACAAACCTGC |
| 951 | CATATCTCAC AGGGACATCA AAAGTAAAAA TGTGCTGTTG AAAAACAACC |
| 1001 | TGACAGCTTG CATTGCTGAC TTTGGGTTGG CCTTAAAATT TGAGGCTGGC |
| 1051 | AAGTCTGCAG GCGATACCCA TGGACAGGTT GGTACCCGGA GGTACATGGC |
| 1101 | TCCAGAGGTA TTAGAGGGTG CTATAAACTT CCAAAGGGAT GCATTTTTGA |
| 1151 | GGATAGATAT GTATGCCATG GGATTAGTCC TATGGGAACT GGCTTCTCGC |
| 1201 | TGTACTGCTG CAGATGGACC TGTAGATGAA TACATGTTGC CATTTGAGGA |
| 1251 | GGAAATTGGC CAGCATCCAT CTCTTGAAGA CATGCAGGAA GTTGTTGTGC |
| 1301 | ATAAAAAAAA GAGGCCTGTT TTAAGAGATT ATTGGCAGAA ACATGCTGGA |
| 1351 | ATGGCAATGC TCTGTGAAAC CATTGAAGAA TGTTGGGATC ACGACGCAGA |
| 1401 | AGCCAGGTTA TCAGCTGGAT GTGTAGGTGA AAGAATTACC CAGATGCAGA |
| 1451 | GACTAACAAA TATTATTACC ACAGAGGACA TTGTAACAGT GGTCACAATG |
| 1501 | GTGACAAATG TTGACTTTCC TCCCAAAGAA TCTAGTCTA |

The nucleic acid sequence encoding processed extracellular ActRIIA polypeptide is as follows:

| (SEQ ID NO: 13) | |
|---|---|
| 1 | ATACTTGGTA GATCAGAAAC TCAGGAGTGT CTTTTCTTTA ATGCTAATTG |
| 51 | GGAAAAAGAC AGAACCAATC AAACTGGTGT TGAACCGTGT TATGGTGACA |
| 101 | AAGATAAACG GCGGCATTGT TTTGCTACCT GGAAGAATAT TTCTGGTTCC |
| 151 | ATTGAAATAG TGAAACAAGG TTGTTGGCTG GATGATATCA ACTGCTATGA |
| 201 | CAGGACTGAT TGTGTAGAAA AAAAAGACAG CCCTGAAGTA TATTTTTGTT |
| 251 | GCTGTGAGGG CAATATGTGT AATGAAAAGT TTTCTTATTT TCCGGAGATG |
| 301 | GAAGTCACAC AGCCCACTTC AAATCCAGTT ACACCTAAGC CACCC |

A general formula for an active (e.g., ligand binding) ActRIIA polypeptide is one that comprises a polypeptide that starts at amino acid 30 and ends at amino acid 110 of SEQ ID NO: 9. Accordingly, ActRIIA polypeptides of the present disclosure may comprise a polypeptide that is at least 80%, 85%, 90%, 95%, 96%, 97%, 98%, 99%, or 100% identical to amino acids 30-110 of SEQ ID NO: 9. Optionally, ActRIIA polypeptides of the present disclosure comprise a polypeptide that is at least 80%, 85%, 90%, 95%, 96%, 97%, 98%, 99%, or 100% identical to amino acids amino acids 12-82 of SEQ ID NO: 9 optionally beginning at a position ranging from 1-5 (e.g., 1, 2, 3, 4, or 5) or 3-5 (e.g., 3, 4, or 5) and ending at a position ranging from 110-116 (e.g., 110, 111, 112, 113, 114, 115, or 116) or 110-115 (e.g., 110, 111, 112, 113, 114, or 115), respectively, and comprising no more than 1, 2, 5, 10 or 15 conservative amino acid changes in the ligand binding pocket, and zero, one or more non-conservative alterations at positions 40, 53, 55, 74, 79 and/or 82 in the ligand-binding pocket with respect to SEQ ID NO: 9.

In certain embodiments, the disclosure relates to heteromultimer complexes that comprise at least one ActRIIA polypeptide, which includes fragments, functional variants, and modified forms thereof. Preferably, ActRIIA polypeptides for use in accordance with inventions of the disclosure (e.g., heteromultimer complexes comprising an ActRIIA polypeptide and uses thereof) are soluble (e.g., an extracellular domain of ActRIIA). In other preferred embodiments, ActRIIA polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one ActRIIA polypeptide that is at least 70%, 75%, 80%, 85%, 90%, 95%, 97%, 98%, or 99% identical to the amino acid sequence of any one of SEQ ID NOs: 9, 10, 11, 118, 120, 409, or 410. In some embodiments, heteromultimer complexes of the disclosure comprise, consist, or consist essentially of at least one ActRIIA polypeptide that is at least 70%, 75%, 80%, 85%, 90%, 95%, 97%, 98%, or 99% identical to the amino acid sequence of any one of SEQ ID NOs: 9, 10, 11, 118, 120, 409, or 410.

In certain aspects, the present disclosure relates to protein complexes that comprise a TGFBRII polypeptide. As used herein, the term “TGFBRII” refers to a family of transforming growth factor-beta receptor II (TGFBRII) proteins from any species and variants derived from such proteins by mutagenesis or other modification. Reference to TGFBRII herein is understood to be a reference to any one of the currently identified forms. Members of the TGFBRII family are generally transmembrane proteins, composed of a ligand-binding extracellular domain with a cysteine-rich region, a transmembrane domain, and a cytoplasmic domain with predicted serine/threonine kinase activity.

The term “TGFBRII polypeptide” includes polypeptides comprising any naturally occurring polypeptide of a TGFBRII family member as well as any variants thereof (including mutants, fragments, fusions, and peptidomimetic forms) that retain a useful activity. Numbering of amino acids for all TGFBRII-related polypeptides described herein is based on the numbering of the human TGFBRII precursor protein sequence below (SEQ ID NO: 42), unless specifically designated otherwise.

The canonical human TGFBRII precursor protein sequence (NCBI Ref Seq NP_003233.4) is as follows:

| (SEQ ID NO: 42) | |
|---|---|
| 1 | MGRGLLRGLW PLHIVLWTRI ASTIPPHVQK SVNNDMIVTD NNGAVKFPQL |
| 51 | CKFCDVRFST CDNQKSCMSN CSITSICEKP QEVCVAVWRK NDENITLETV |
| 101 | CHDPKLPYHD FILEDAASPK CIMKEKKKPG ETFFMCSCSS DECNDNIIFS |
| 151 | EEYNTSNPDL LLVIFQVTGI SLLPPLGVAI SVIIIFYCYR VNRQQKLSST |
| 201 | WETGKTRKLM EFSEHCAIIL EDDRSDISST CANNINHNTE LLPIELDTLV |
| 251 | GKGRFAEVYK AKLKQNTSEQ FETVAVKIFP YEEYASWKTE KDIFSDINLK |
| 301 | HENILQFLTA EERKTELGKQ YWLITAFHAK GNLQEYLTRH VISWEDLRKL |
| 351 | GSSLARGIAH LHSDHTPCGR PKMPIVHRDL KSSNILVKND LTCCLCDFGL |
| 401 | SLRLDPTLSV DDLANSGQVG TARYMAPEVL ESRMNLENVE SFKQTDVYSM |
| 451 | ALVLWEMTSR CNAVGEVKDY EPPFGSKVRE HPCVESMKDN VLRDRGRPEI |
| 501 | PSFWLNHQGI QMVCETLTEC WDHDPEARLT AQCVAERFSE LEHLDRLSGR |
| 551 | SCSEEKIPED GSLNTTK |

The signal peptide is indicated by a single underline and the extracellular domain is indicated in bold font.

The processed extracellular TGFBRII polypeptide sequence is as follows:

(SEQ ID NO: 43)
TIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKCI
MKEKKKPGETFFMCSCSSDECNDNIIFSEEYNTSNPDLLLVIFQ

The nucleic acid sequence encoding TGFBRII precursor protein is shown below (SEQ ID NO:44), corresponding to nucleotides 383-2083 of Genbank Reference Sequence NM_003242.5. The signal sequence is underlined.

(SEQ ID NO: 44)
ATGGGTCGGGGGCTGCTCAGGGGCCTGTGGCCGCTGCACATCGTCCTGTG
GACGCGTATCGCCAGCACGATCCCACCGCACGTTCAGAAGTCGGTTAATA
ACGACATGATAGTCACTGACAACAACGGTGCAGTCAAGTTTCCACAACTG
TGTAAATTTTGTGATGTGAGATTTTCCACCTGTGACAACCAGAAATCCTG
CATGAGCAACTGCAGCATCACCTCCATCTGTGAGAAGCCACAGGAAGTCT
GTGTGGCTGTATGGAGAAAGAATGACGAGAACATAACACTAGAGACAGTT
TGCCATGACCCCAAGCTCCCCTACCATGACTTTATTCTGGAAGATGCTGC
TTCTCCAAAGTGCATTATGAAGGAAAAAAAAAAGCCTGGTGAGACTTTCT
TCATGTGTTCCTGTAGCTCTGATGAGTGCAATGACAACATCATCTTCTCA
GAAGAATATAACACCAGCAATCCTGACTTGTTGCTAGTCATATTTCAAGT
GACAGGCATCAGCCTCCTGCCACCACTGGGAGTTGCCATATCTGTCATCA
TCATCTTCTACTGCTACCGCGTTAACCGGCAGCAGAAGCTGAGTTCAACC
TGGGAAACCGGCAAGACGCGGAAGCTCATGGAGTTCAGCGAGCACTGTGC
CATCATCCTGGAAGATGACCGCTCTGACATCAGCTCCACGTGTGCCAACA
ACATCAACCACAACACAGAGCTGCTGCCCATTGAGCTGGACACCCTGGTG
GGGAAAGGTCGCTTTGCTGAGGTCTATAAGGCCAAGCTGAAGCAGAACAC
TTCAGAGCAGTTTGAGACAGTGGCAGTCAAGATCTTTCCCTATGAGGAGT
ATGCCTCTTGGAAGACAGAGAAGGACATCTTCTCAGACATCAATCTGAAG
CATGAGAACATACTCCAGTTCCTGACGGCTGAGGAGCGGAAGACGGAGTT
GGGGAAACAATACTGGCTGATCACCGCCTTCCACGCCAAGGGCAACCTAC
AGGAGTACCTGACGCGGCATGTCATCAGCTGGGAGGACCTGCGCAAGCTG
GGCAGCTCCCTCGCCCGGGGGATTGCTCACCTCCACAGTGATCACACTCC
ATGTGGGAGGCCCAAGATGCCCATCGTGCACAGGGACCTCAAGAGCTCCA
ATATCCTCGTGAAGAACGACCTAACCTGCTGCCTGTGTGACTTTGGGCTT
TCCCTGCGTCTGGACCCTACTCTGTCTGTGGATGACCTGGCTAACAGTGG
GCAGGTGGGAACTGCAAGATACATGGCTCCAGAAGTCCTAGAATCCAGGA
TGAATTTGGAGAATGTTGAGTCCTTCAAGCAGACCGATGTCTACTCCATG
GCTCTGGTGCTCTGGGAAATGACATCTCGCTGTAATGCAGTGGGAGAAGT
AAAAGATTATGAGCCTCCATTTGGTTCCAAGGTGCGGGAGCACCCCTGTG
TCGAAAGCATGAAGGACAACGTGTTGAGAGATCGAGGGCGACCAGAAATT
CCCAGCTTCTGGCTCAACCACCAGGGCATCCAGATGGTGTGTGAGACGTT
GACTGAGTGCTGGGACCACGACCCAGAGGCCCGTCTCACAGCCCAGTGTG
TGGCAGAACGCTTCAGTGAGCTGGAGCATCTGGACAGGCTCTCGGGGAGG
AGCTGCTCGGAGGAGAAGATTCCTGAAGACGGCTCCCTAAACACTACCAA
A

The nucleic acid sequence encoding processed extracellular TGFBRII polypeptide is as follows:

(SEQ ID NO: 45)
ACGATCCCACCGCACGTTCAGAAGTCGGTTAATAACGACATGATAGTCAC
TGACAACAACGGTGCAGTCAAGTTTCCACAACTGTGTAAATTTTGTGATG
TGAGATTTTCCACCTGTGACAACCAGAAATCCTGCATGAGCAACTGCAGC
ATCACCTCCATCTGTGAGAAGCCACAGGAAGTCTGTGTGGCTGTATGGAG
AAAGAATGACGAGAACATAACACTAGAGACAGTTTGCCATGACCCCAAGC
TCCCCTACCATGACTTTATTCTGGAAGATGCTGCTTCTCCAAAGTGCATT
ATGAAGGAAAAAAAAAAGCCTGGTGAGACTTTCTTCATGTGTTCCTGTAG
CTCTGATGAGTGCAATGACAACATCATCTTCTCAGAAGAATATAACACCA
GCAATCCTGACTTGTTGCTAGTCATATTTCAA

An alternative isoform of TGFBRII, isoform A (NP_001020018.1), is as follows:

| (SEQ ID NO: 67) | |
|---|---|
| 1 | MGRGLLRGLW PLHIVLWTRI ASTIPPHVQK SDVEMEAQKD EIICPSCNRT |
| 51 | AHPLRHINND MIVTDNNGAV KFPQLCKFCD VRFSTCDNQK SCMSNCSITS |
| 101 | ICEKPQEVCV AVWRKNDENI TLETVCHDPK LPYHDFILED AASPKCIMKE |
| 151 | KKKPGETFFM CSCSSDECND NIIFSEEYNT SNPDLLLVIF QVTGISLLPP |
| 201 | LGVAISVIII FYCYRVNRQQ KLSSTWETGK TRKLMEFSEH CAIILEDDRS |
| 251 | DISSTCANNI NHNTELLPIE LDTLVGKGRF AEVYKAKLKQ NTSEQFETVA |
| 301 | VKIFPYEEYA SWKTEKDIFS DINLKHENIL QFLTAEERKT ELGKQYWLIT |
| 351 | AFHAKGNLQE YLTRHVISWE DLRKLGSSLA RGIAHLHSDH TPCGRPKMPI |
| 401 | VHRDLKSSNI LVKNDLTCCL CDFGLSLRLD PTLSVDDLAN SGQVGTARYM |
| 451 | APEVLESRMN LENVESFKQT DVYSMALVLW EMTSRCNAVG EVKDYEPPFG |
| 501 | SKVREHPCVE SMKDNVLRDR GRPEIPSFWL NHQGIQMVCE TLTECWDHDP |
| 551 | EARLTAQCVA ERFSELEHLD RLSGRSCSEE KIPEDGSLNT TK |

The signal peptide is indicated by a single underline and the extracellular domain is indicated in bold font.

The processed extracellular TGFBRII polypeptide sequence (isoform A) is as follows:

(SEQ ID NO: 68)
TIPPHVQKSDVEMEAQKDEIICPSCNRTAHPLRHINNDMIVTDNNGAVKF
PQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNI
IFSEEYNTSNPDLLLVIFQ

A nucleic acid sequence encoding the TGFBRII precursor protein (isoform A) is shown below (SEQ ID NO: 69), corresponding to nucleotides 383-2158 of Genbank Reference Sequence NM_001024847.2. The signal sequence is underlined.

(SEQ ID NO: 69)
ATGGGTCGGGGGCTGCTCAGGGGCCTGTGGCCGCTGCACATCGTCCTGTG
GACGCGTATCGCCAGCACGATCCCACCGCACGTTCAGAAGTCGGATGTGG
AAATGGAGGCCCAGAAAGATGAAATCATCTGCCCCAGCTGTAATAGGACT
GCCCATCCACTGAGACATATTAATAACGACATGATAGTCACTGACAACAA
CGGTGCAGTCAAGTTTCCACAACTGTGTAAATTTTGTGATGTGAGATTTT
CCACCTGTGACAACCAGAAATCCTGCATGAGCAACTGCAGCATCACCTCC
ATCTGTGAGAAGCCACAGGAAGTCTGTGTGGCTGTATGGAGAAAGAATGA
CGAGAACATAACACTAGAGACAGTTTGCCATGACCCCAAGCTCCCCTACC
ATGACTTTATTCTGGAAGATGCTGCTTCTCCAAAGTGCATTATGAAGGAA
AAAAAAAAGCCTGGTGAGACTTTCTTCATGTGTTCCTGTAGCTCTGATGA
GTGCAATGACAACATCATCTTCTCAGAAGAATATAACACCAGCAATCCTG
ACTTGTTGCTAGTCATATTTCAAGTGACAGGCATCAGCCTCCTGCCACCA
CTGGGAGTTGCCATATCTGTCATCATCATCTTCTACTGCTACCGCGTTAA
CCGGCAGCAGAAGCTGAGTTCAACCTGGGAAACCGGCAAGACGCGGAAGC
TCATGGAGTTCAGCGAGCACTGTGCCATCATCCTGGAAGATGACCGCTCT
GACATCAGCTCCACGTGTGCCAACAACATCAACCACAACACAGAGCTGCT
GCCCATTGAGCTGGACACCCTGGTGGGGAAAGGTCGCTTTGCTGAGGTCT
ATAAGGCCAAGCTGAAGCAGAACACTTCAGAGCAGTTTGAGACAGTGGCA
GTCAAGATCTTTCCCTATGAGGAGTATGCCTCTTGGAAGACAGAGAAGGA
CATCTTCTCAGACATCAATCTGAAGCATGAGAACATACTCCAGTTCCTGA
CGGCTGAGGAGCGGAAGACGGAGTTGGGGAAACAATACTGGCTGATCACC
GCCTTCCACGCCAAGGGCAACCTACAGGAGTACCTGACGCGGCATGTCAT
CAGCTGGGAGGACCTGCGCAAGCTGGGCAGCTCCCTCGCCCGGGGGATTG
CTCACCTCCACAGTGATCACACTCCATGTGGGAGGCCCAAGATGCCCATC
GTGCACAGGGACCTCAAGAGCTCCAATATCCTCGTGAAGAACGACCTAAC
CTGCTGCCTGTGTGACTTTGGGCTTTCCCTGCGTCTGGACCCTACTCTGT
CTGTGGATGACCTGGCTAACAGTGGGCAGGTGGGAACTGCAAGATACATG
GCTCCAGAAGTCCTAGAATCCAGGATGAATTTGGAGAATGTTGAGTCCTT
CAAGCAGACCGATGTCTACTCCATGGCTCTGGTGCTCTGGGAAATGACAT
CTCGCTGTAATGCAGTGGGAGAAGTAAAAGATTATGAGCCTCCATTTGGT
TCCAAGGTGCGGGAGCACCCCTGTGTCGAAAGCATGAAGGACAACGTGTT
GAGAGATCGAGGGCGACCAGAAATTCCCAGCTTCTGGCTCAACCACCAGG
GCATCCAGATGGTGTGTGAGACGTTGACTGAGTGCTGGGACCACGACCCA
GAGGCCCGTCTCACAGCCCAGTGTGTGGCAGAACGCTTCAGTGAGCTGGA
GCATCTGGACAGGCTCTCGGGGAGGAGCTGCTCGGAGGAGAAGATTCCTG
AAGACGGCTCCCTAAACACTACCAAA

A nucleic acid sequence encoding the processed extracellular TGFBRII polypeptide (isoform A) is as follows:

(SEQ ID NO: 70)
ACGATCCCACCGCACGTTCAGAAGTCGGATGTGGAAATGGAGGCCCAGAA
AGATGAAATCATCTGCCCCAGCTGTAATAGGACTGCCCATCCACTGAGAC
ATATTAATAACGACATGATAGTCACTGACAACAACGGTGCAGTCAAGTTT
CCACAACTGTGTAAATTTTGTGATGTGAGATTTTCCACCTGTGACAACCA
GAAATCCTGCATGAGCAACTGCAGCATCACCTCCATCTGTGAGAAGCCAC
AGGAAGTCTGTGTGGCTGTATGGAGAAAGAATGACGAGAACATAACACTA
GAGACAGTTTGCCATGACCCCAAGCTCCCCTACCATGACTTTATTCTGGA
AGATGCTGCTTCTCCAAAGTGCATTATGAAGGAAAAAAAAAAGCCTGGTG
AGACTTTCTTCATGTGTTCCTGTAGCTCTGATGAGTGCAATGACAACATC
ATCTTCTCAGAAGAATATAACACCAGCAATCCTGACTTGTTGCTAGTCAT
ATTTCAA.

Either of the foregoing TGFβRII isoforms (SEQ ID NOs: 42, 43, 67, and 68) could incorporate an insertion of 36 amino acids (SEQ ID NO: 95) between the pair of glutamate residues (positions 151 and 152 of SEQ ID NO: 42; positions 129 and 130 of SEQ ID NO: 43; positions 176 and 177 of SEQ ID NO: 67; or positions 154 and 155 of SEQ ID NO: 68) located near the C-terminus of the TGFβRII ECD, as occurs naturally in the TGFβRII isoform C (Konrad et al., BMC Genomics 8:318, 2007).

(SEQ ID NO: 95)
GRCKIRHIGS NNRLQRSTCQ NTGWESAHVM KTPGFR

In certain embodiments, the disclosure relates to heteromultimer complexes that comprise at least one TGFBRII polypeptide, which includes fragments, functional variants, and modified forms thereof. Preferably, TGFBRII polypeptides for use in accordance with inventions of the disclosure (e.g., heteromultimer complexes comprising a TGFBRII polypeptide and uses thereof) are soluble (e.g., an extracellular domain of TGFβRII). In other preferred embodiments, TGFBRII polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one TGFBRII polypeptide that is at least 70%, 75%, 80%, 85%, 90%, 95%, 97%, 98%, or 99% identical to the amino acid sequence of SEQ ID NOs: 42, 43, 67, or 68, with or without insertion of SEQ ID NO: 95 as described above. In some embodiments, heteromultimer complexes of the disclosure consist or consist essentially of at least one TGFBRII polypeptide that is at least 70%, 75%, 80%, 85%, 90%, 95%, 97%, 98%, or 99% identical to the amino acid sequence of SEQ ID NOs: 42, 43, 67, or 68, with or without insertion of SEQ ID NO: 95.

In certain aspects, the present disclosure relates to protein complexes that comprise a BMPRII polypeptide. As used herein, the term “BMPRII” refers to a family of bone morphogenetic protein receptor type II (BMPRII) proteins from any species and variants derived from such BMPRII proteins by mutagenesis or other modification. Reference to BMPRII herein is understood to be a reference to any one of the currently identified forms. Members of the BMPRII family are generally transmembrane proteins, composed of a ligand-binding extracellular domain with a cysteine-rich region, a transmembrane domain, and a cytoplasmic domain with predicted serine/threonine kinase activity.

The term “BMPRII polypeptide” includes polypeptides comprising any naturally occurring polypeptide of a BMPRII family member as well as any variants thereof (including mutants, fragments, fusions, and peptidomimetic forms) that retain a useful activity. Numbering of amino acids for all BMPRII-related polypeptides described herein is based on the numbering of the human BMPRII precursor protein sequence below (SEQ ID NO: 46), unless specifically designated otherwise.

The canonical human BMPRII precursor protein sequence (NCBI Ref Seq NP_001195.2) is as follows:

| (SEQ ID NO: 46) | |
|---|---|
| 1 | MTSSLQRPWR VPWLPWTILL VSTAAASQNQ ERLCAFKDPY QQDLGIGESR |
| 51 | ISHENGTILC SKGSTCYGLW EKSKGDINLV KQGCWSHIGD PQECHYEECV |
| 101 | VTTTPPSIQN GTYRFCCCST DLCNVNFTEN FPPPDTTPLS PPHSFNRDET |
| 151 | IIIALASVSV LAVLIVALCF GYRMLTGDRK QGLHSMNMME AAASEPSLDL |
| 201 | DNLKLLELIG RGRYGAVYKG SLDERPVAVK VFSFANRQNF INEKNIYRVP |
| 251 | LMEHDNIARF IVGDERVTAD GRMEYLLVME YYPNGSLCKY LSLHTSDWVS |
| 301 | SCRLAHSVTR GLAYLHTELP RGDHYKPAIS HRDLNSRNVL VKNDGTCVIS |
| 351 | DFGLSMRLTG NRLVRPGEED NAAISEVGTI RYMAPEVLEG AVNLRDCESA |
| 401 | LKQVDMYALG LIYWEIFMRC TDLFPGESVP EYQMAFQTEV GNHPTFEDMQ |
| 451 | VLVSREKQRP KFPEAWKENS LAVRSLKETI EDCWDQDAEA RLTAQCAEER |
| 501 | MAELMMIWER NKSVSPTVNP MSTAMQNERN LSHNRRVPKI GPYPDYSSSS |
| 551 | YIEDSIHHTD SIVKNISSEH SMSSTPLTIG EKNRNSINYE RQQAQARIPS |
| 601 | PETSVTSLST NTTTTNTTGL TPSTGMTTIS EMPYPDETNL HTTNVAQSIG |
| 651 | PTPVCLQLTE EDLETNKLDP KEVDKNLKES SDENLMEHSL KQFSGPDPLS |
| 701 | STSSSLLYPL IKLAVEATGQ QDFTQTANGQ ACLIPDVLPT QIYPLPKQQN |
| 751 | LPKRPTSLPL NTKNSTKEPR LKFGSKHKSN LKQVETGVAK MNTINAAEPH |
| 801 | VVTVTMNGVA GRNHSVNSHA ATTQYANGTV LSGQTTNIVT HRAQEMLQNQ |
| 851 | FIGEDTRLNI NSSPDEHEPL LRREQQAGHD EGVLDRLVDR RERPLEGGRT |
| 901 | NSNNNNSNPC SEQDVLAQGV PSTAADPGPS KPRRAQRPNS LDLSATNVLD |
| 951 | GSSIQIGEST QDGKSGSGEK IKKRVKTPYS LKRWRPSTWV ISTESLDCEV |
| 1001 | NNNGSNRAVH SKSSTAVYLA EGGTATTMVS KDIGMNCL |

The signal peptide is indicated by a single underline and the extracellular domain is indicated in bold font.

The processed extracellular BMPRII polypeptide sequence is as follows:

(SEQ ID NO: 47)
SQNQERLCAFKDPYQQDLGIGESRISHENGTILCSKGSTCYGLWEKSKGD
INLVKQGCWSHIGDPQECHYEECVVTTTPPSIQNGTYRFCCCSTDLCNVN
FTENFPPPDTTPLSPPHSFNRDET

A nucleic acid sequence encoding BMPRII precursor protein is shown below (SEQ ID NO: 48), as follows nucleotides 1149-4262 of Genbank Reference Sequence NM_001204.6. The signal sequence is underlined.

(SEQ ID NO: 48)
ATGACTTCCTCGCTGCAGCGGCCCTGGCGGGTGCCCTGGCTACCATGGAC
CATCCTGCTGGTCAGCACTGCGGCTGCTTCGCAGAATCAAGAACGGCTAT
GTGCGTTTAAAGATCCGTATCAGCAAGACCTTGGGATAGGTGAGAGTAGA
ATCTCTCATGAAAATGGGACAATATTATGCTCGAAAGGTAGCACCTGCTA
TGGCCTTTGGGAGAAATCAAAAGGGGACATAAATCTTGTAAAACAAGGAT
GTTGGTCTCACATTGGAGATCCCCAAGAGTGTCACTATGAAGAATGTGTA
GTAACTACCACTCCTCCCTCAATTCAGAATGGAACATACCGTTTCTGCTG
TTGTAGCACAGATTTATGTAATGTCAACTTTACTGAGAATTTTCCACCTC
CTGACACAACACCACTCAGTCCACCTCATTCATTTAACCGAGATGAGACA
ATAATCATTGCTTTGGCATCAGTCTCTGTATTAGCTGTTTTGATAGTTGC
CTTATGCTTTGGATACAGAATGTTGACAGGAGACCGTAAACAAGGTCTTC
ACAGTATGAACATGATGGAGGCAGCAGCATCCGAACCCTCTCTTGATCTA
GATAATCTGAAACTGTTGGAGCTGATTGGCCGAGGTCGATATGGAGCAGT
ATATAAAGGCTCCTTGGATGAGCGTCCAGTTGCTGTAAAAGTGTTTTCCT
TTGCAAACCGTCAGAATTTTATCAACGAAAAGAACATTTACAGAGTGCCT
TTGATGGAACATGACAACATTGCCCGCTTTATAGTTGGAGATGAGAGAGT
CACTGCAGATGGACGCATGGAATATTTGCTTGTGATGGAGTACTATCCCA
ATGGATCTTTATGCAAGTATTTAAGTCTCCACACAAGTGACTGGGTAAGC
TCTTGCCGTCTTGCTCATTCTGTTACTAGAGGACTGGCTTATCTTCACAC
AGAATTACCACGAGGAGATCATTATAAACCTGCAATTTCCCATCGAGATT
TAAACAGCAGAAATGTCCTAGTGAAAAATGATGGAACCTGTGTTATTAGT
GACTTTGGACTGTCCATGAGGCTGACTGGAAATAGACTGGTGCGCCCAGG
GGAGGAAGATAATGCAGCCATAAGCGAGGTTGGCACTATCAGATATATGG
CACCAGAAGTGCTAGAAGGAGCTGTGAACTTGAGGGACTGTGAATCAGCT
TTGAAACAAGTAGACATGTATGCTCTTGGACTAATCTATTGGGAGATATT
TATGAGATGTACAGACCTCTTCCCAGGGGAATCCGTACCAGAGTACCAGA
TGGCTTTTCAGACAGAGGTTGGAAACCATCCCACTTTTGAGGATATGCAG
GTTCTCGTGTCTAGGGAAAAACAGAGACCCAAGTTCCCAGAAGCCTGGAA
AGAAAATAGCCTGGCAGTGAGGTCACTCAAGGAGACAATCGAAGACTGTT
GGGACCAGGATGCAGAGGCTCGGCTTACTGCACAGTGTGCTGAGGAAAGG
ATGGCTGAACTTATGATGATTTGGGAAAGAAACAAATCTGTGAGCCCAAC
AGTCAATCCAATGTCTACTGCTATGCAGAATGAACGCAACCTGTCACATA
ATAGGCGTGTGCCAAAAATTGGTCCTTATCCAGATTATTCTTCCTCCTCA
TACATTGAAGACTCTATCCATCATACTGACAGCATCGTGAAGAATATTTC
CTCTGAGCATTCTATGTCCAGCACACCTTTGACTATAGGGGAAAAAAACC
GAAATTCAATTAACTATGAACGACAGCAAGCACAAGCTCGAATCCCCAGC
CCTGAAACAAGTGTCACCAGCCTCTCCACCAACACAACAACCACAAACAC
CACAGGACTCACGCCAAGTACTGGCATGACTACTATATCTGAGATGCCAT
ACCCAGATGAAACAAATCTGCATACCACAAATGTTGCACAGTCAATTGGG
CCAACCCCTGTCTGCTTACAGCTGACAGAAGAAGACTTGGAAACCAACAA
GCTAGACCCAAAAGAAGTTGATAAGAACCTCAAGGAAAGCTCTGATGAGA
ATCTCATGGAGCACTCTCTTAAACAGTTCAGTGGCCCAGACCCACTGAGC
AGTACTAGTTCTAGCTTGCTTTACCCACTCATAAAACTTGCAGTAGAAGC
AACTGGACAGCAGGACTTCACACAGACTGCAAATGGCCAAGCATGTTTGA
TTCCTGATGTTCTGCCTACTCAGATCTATCCTCTCCCCAAGCAGCAGAAC
CTTCCCAAGAGACCTACTAGTTTGCCTTTGAACACCAAAAATTCAACAAA
AGAGCCCCGGCTAAAATTTGGCAGCAAGCACAAATCAAACTTGAAACAAG
TCGAAACTGGAGTTGCCAAGATGAATACAATCAATGCAGCAGAACCTCAT
GTGGTGACAGTCACCATGAATGGTGTGGCAGGTAGAAACCACAGTGTTAA
CTCCCATGCTGCCACAACCCAATATGCCAATGGGACAGTACTATCTGGCC
AAACAACCAACATAGTGACACATAGGGCCCAAGAAATGTTGCAGAATCAG
TTTATTGGTGAGGACACCCGGCTGAATATTAATTCCAGTCCTGATGAGCA
TGAGCCTTTACTGAGACGAGAGCAACAAGCTGGCCATGATGAAGGTGTTC
TGGATCGTCTTGTGGACAGGAGGGAACGGCCACTAGAAGGTGGCCGAACT
AATTCCAATAACAACAACAGCAATCCATGTTCAGAACAAGATGTTCTTGC
ACAGGGTGTTCCAAGCACAGCAGCAGATCCTGGGCCATCAAAGCCCAGAA
GAGCACAGAGGCCTAATTCTCTGGATCTTTCAGCCACAAATGTCCTGGAT
GGCAGCAGTATACAGATAGGTGAGTCAACACAAGATGGCAAATCAGGATC
AGGTGAAAAGATCAAGAAACGTGTGAAAACTCCCTATTCTCTTAAGCGGT
GGCGCCCCTCCACCTGGGTCATCTCCACTGAATCGCTGGACTGTGAAGTC
AACAATAATGGCAGTAACAGGGCAGTTCATTCCAAATCCAGCACTGCTGT
TTACCTTGCAGAAGGAGGCACTGCTACAACCATGGTGTCTAAAGATATAG
GAATGAACTGTCTG

The nucleic acid sequence encoding the extracellular BMPRII polypeptide is as follows:

(SEQ ID NO: 49)
TCGCAGAATCAAGAACGGCTATGTGCGTTTAAAGATCCGTATCAGCAAGA
CCTTGGGATAGGTGAGAGTAGAATCTCTCATGAAAATGGGACAATATTAT
GCTCGAAAGGTAGCACCTGCTATGGCCTTTGGGAGAAATCAAAAGGGGAC
ATAAATCTTGTAAAACAAGGATGTTGGTCTCACATTGGAGATCCCCAAGA
GTGTCACTATGAAGAATGTGTAGTAACTACCACTCCTCCCTCAATTCAGA
ATGGAACATACCGTTTCTGCTGTTGTAGCACAGATTTATGTAATGTCAAC
TTTACTGAGAATTTTCCACCTCCTGACACAACACCACTCAGTCCACCTCA
TTCATTTAACCGAGATGAGACA

An alternative isoform of BMPRII, isoform 2 (GenBank: AAA86519.1) is as follows:

| (SEQ ID NO: 71) | |
|---|---|
| 1 | MTSSLQRPWR VPWLPWTILL VSTAAASQNQ ERLCAFKDPY QQDLGIGESR |
| 51 | ISHENGTILC SKGSTCYGLW EKSKGDINLV KQGCWSHIGD PQECHYEECV |
| 101 | VTTTPPSIQN GTYRFCCCST DLCNVNFTEN FPPPDTTPLS PPHSFNRDET |
| 151 | IIIALASVSV LAVLIVALCF GYRMLTGDRK QGLHSMNMME AAASEPSLDL |
| 201 | DNLKLLELIG RGRYGAVYKG SLDERPVAVK VFSFANRQNF INEKNIYRVP |
| 251 | LMEHDNIARF IVGDERVTAD GRMEYLLVME YYPNGSLCKY LSLHTSDWVS |
| 301 | SCRLAHSVTR GLAYLHTELP RGDHYKPAIS HRDLNSRNVL VKNDGTCVIS |
| 351 | DFGLSMRLTG NRLVRPGEED NAAISEVGTI RYMAPEVLEG AVNLRDCESA |
| 401 | LKQVDMYALG LIYWEIFMRC TDLFPGESVP EYQMAFQTEV GNHPTFEDMQ |
| 451 | VLVSREKQRP KFPEAWKENS LAVRSLKETI EDCWDQDAEA RLTAQCAEER |
| 501 | MAELMMIWER NKSVSPTVNP MSTAMQNERR |

The signal peptide is indicated by a single underline and the extracellular domain is indicated in bold font.

The processed extracellular BMPRII polypeptide sequence (isoform 2) is as follows:

(SEQ ID NO: 72)
SQNQERLCAFKDPYQQDLGIGESRISHENGTILCSKGSTCYGLWEKSKGD
INLVKQGCWSHIGDPQECHYEECVVTTTPPSIQNGTYRFCCCSTDLCNVN
FTENFPPPDTTPLSPPHSFNRDET

A nucleic acid sequence encoding human BMPRII precursor protein (isoform 2) is shown below (SEQ ID NO:73), corresponding to nucleotides 163-1752 of Genbank Reference Sequence U25110.1. The signal sequence is underlined.

(SEQ ID NO: 73)
ATGACTTCCTCGCTGCAGCGGCCCTGGCGGGTGCCCTGGCTACCATGGAC
CATCCTGCTGGTCAGCACTGCGGCTGCTTCGCAGAATCAAGAACGGCTAT
GTGCGTTTAAAGATCCGTATCAGCAAGACCTTGGGATAGGTGAGAGTAGA
ATCTCTCATGAAAATGGGACAATATTATGCTCGAAAGGTAGCACCTGCTA
TGGCCTTTGGGAGAAATCAAAAGGGGACATAAATCTTGTAAAACAAGGAT
GTTGGTCTCACATTGGAGATCCCCAAGAGTGTCACTATGAAGAATGTGTA
GTAACTACCACTCCTCCCTCAATTCAGAATGGAACATACCGTTTCTGCTG
TTGTAGCACAGATTTATGTAATGTCAACTTTACTGAGAATTTTCCACCTC
CTGACACAACACCACTCAGTCCACCTCATTCATTTAACCGAGATGAGACA
ATAATCATTGCTTTGGCATCAGTCTCTGTATTAGCTGTTTTGATAGTTGC
CTTATGCTTTGGATACAGAATGTTGACAGGAGACCGTAAACAAGGTCTTC
ACAGTATGAACATGATGGAGGCAGCAGCATCCGAACCCTCTCTTGATCTA
GATAATCTGAAACTGTTGGAGCTGATTGGCCGAGGTCGATATGGAGCAGT
ATATAAAGGCTCCTTGGATGAGCGTCCAGTTGCTGTAAAAGTGTTTTCCT
TTGCAAACCGTCAGAATTTTATCAACGAAAAGAACATTTACAGAGTGCCT
TTGATGGAACATGACAACATTGCCCGCTTTATAGTTGGAGATGAGAGAGT
CACTGCAGATGGACGCATGGAATATTTGCTTGTGATGGAGTACTATCCCA
ATGGATCTTTATGCAAGTATTTAAGTCTCCACACAAGTGACTGGGTAAGC
TCTTGCCGTCTTGCTCATTCTGTTACTAGAGGACTGGCTTATCTTCACAC
AGAATTACCACGAGGAGATCATTATAAACCTGCAATTTCCCATCGAGATT
TAAACAGCAGAAATGTCCTAGTGAAAAATGATGGAACCTGTGTTATTAGT
GACTTTGGACTGTCCATGAGGCTGACTGGAAATAGACTGGTGCGCCCAGG
GGAGGAAGATAATGCAGCCATAAGCGAGGTTGGCACTATCAGATATATGG
CACCAGAAGTGCTAGAAGGAGCTGTGAACTTGAGGGACTGTGAATCAGCT
TTGAAACAAGTAGACATGTATGCTCTTGGACTAATCTATTGGGAGATATT
TATGAGATGTACAGACCTCTTCCCAGGGGAATCCGTACCAGAGTACCAGA
TGGCTTTTCAGACAGAGGTTGGAAACCATCCCACTTTTGAGGATATGCAG
GTTCTCGTGTCTAGGGAAAAACAGAGACCCAAGTTCCCAGAAGCCTGGAA
AGAAAATAGCCTGGCAGTGAGGTCACTCAAGGAGACAATCGAAGACTGTT
GGGACCAGGATGCAGAGGCTCGGCTTACTGCACAGTGTGCTGAGGAAAGG
ATGGCTGAACTTATGATGATTTGGGAAAGAAACAAATCTGTGAGCCCAAC
AGTCAATCCAATGTCTACTGCTATGCAGAATGAACGTAGG

A nucleic acid sequence encoding an extracellular BMPRII polypeptide (isoform 2) is as follows:

(SEQ ID NO: 74)
TCGCAGAATCAAGAACGGCTATGTGCGTTTAAAGATCCGTATCAGCAAGA
CCTTGGGATAGGTGAGAGTAGAATCTCTCATGAAAATGGGACAATATTAT
GCTCGAAAGGTAGCACCTGCTATGGCCTTTGGGAGAAATCAAAAGGGGAC
ATAAATCTTGTAAAACAAGGATGTTGGTCTCACATTGGAGATCCCCAAGA
GTGTCACTATGAAGAATGTGTAGTAACTACCACTCCTCCCTCAATTCAGA
ATGGAACATACCGTTTCTGCTGTTGTAGCACAGATTTATGTAATGTCAAC
TTTACTGAGAATTTTCCACCTCCTGACACAACACCACTCAGTCCACCTCA
TTCATTTAACCGAGATGAGACA

In certain embodiments, the disclosure relates to heteromultimer complexes that comprise at least one BMPRII polypeptide, which includes fragments, functional variants, and modified forms thereof. Preferably, BMPRII polypeptides for use in accordance with inventions of the disclosure (e.g., heteromultimer complexes comprising a BMPRII polypeptide and uses thereof) are soluble (e.g., an extracellular domain of BMPRII). In other preferred embodiments, BMPRII polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one BMPRII polypeptide that is at least 70%, 75%, 80%, 85%, 90%, 95%, 97%, 98%, or 99% identical to the amino acid sequence of SEQ ID NO: 46, 47, 71, 72, 121, 123, 411, or 412. In some embodiments, heteromultimer complexes of the disclosure consist or consist essentially of at least one BMPRII polypeptide that is at least 70%, 75%, 80%, 85%, 90%, 95%, 97%, 98%, or 99% identical to the amino acid sequence of SEQ ID NO: 46, 47, 71, 72, 121, 123, 411, or 412.

In certain aspects, the present disclosure relates to protein complexes that comprise an MISRII polypeptide. As used herein, the term “MISRII” refers to a family of Müllerian inhibiting substance receptor type II (MISRII) proteins from any species and variants derived from such MISRII proteins by mutagenesis or other modification. Reference to MISRII herein is understood to be a reference to any one of the currently identified forms. Members of the MISRII family are generally transmembrane proteins, composed of a ligand-binding extracellular domain with a cysteine-rich region, a transmembrane domain, and a cytoplasmic domain with predicted serine/threonine kinase activity.

The term “MISRII polypeptide” includes polypeptides comprising any naturally occurring polypeptide of an MISRII family member as well as any variants thereof (including mutants, fragments, fusions, and peptidomimetic forms) that retain a useful activity. Numbering of amino acids for all MISRII-related polypeptides described herein is based on the numbering of the human MISRII precursor protein sequence below (SEQ ID NO: 50), unless specifically designated otherwise.

The canonical human MISRII precursor protein sequence (NCBI Ref Seq NP_065434.1) is as follows:

| (SEQ ID NO: 50) | |
|---|---|
| 1 | MLGSLGLWAL LPTAVEAPPN RRTCVFFEAP GVRGSTKTLG ELLDTGTELP |
| 51 | RAIRCLYSRC CFGIWNLTQD RAQVEMQGCR DSDEPGCESL HCDPSPRAHP |
| 101 | SPGSTLFTCS CGTDFCNANY SHLPPPGSPG TPGSQGPQAA PGESIWMALV |
| 151 | LLGLFLLLLL LLGSIILALL QRKNYRVRGE PVPEPRPDSG RDWSVELQEL |
| 201 | PELCFSQVIR EGGHAVVWAG QLQGKLVAIK AFPPRSVAQF QAERALYELP |
| 251 | GLQHDHIVRF ITASRGGPGR LLSGPLLVLE LHPKGSLCHY LTQYTSDWGS |
| 301 | SLRMALSLAQ GLAFLHEERW QNGQYKPGIA HRDLSSQNVL IREDGSCAIG |
| 351 | DLGLALVLPG LTQPPAWTPT QPQGPAAIME AGTQRYMAPE LLDKTLDLQD |
| 401 | WGMALRRADI YSLALLLWEI LSRCPDLRPD SSPPPFQLAY EAELGNTPTS |
| 451 | DELWALAVQE RRRPYIPSTW RCFATDPDGL RELLEDCWDA DPEARLTAEC |
| 501 | VQQRLAALAH PQESHPFPES CPRGCPPLCP EDCTSIPAPT ILPCRPQRSA |
| 551 | CHFSVQQGPC SRNPQPACTL SPV |

The signal peptide is indicated by a single underline and the extracellular domain is indicated in bold font.

The processed extracellular MISRII polypeptide sequence is as follows:

(SEQ ID NO: 51)
PPNRRTCVFFEAPGVRGSTKTLGELLDTGTELPRAIRCLYSRCCFGIWNL
TQDRAQVEMQGCRDSDEPGCESLHCDPSPRAHPSPGSTLFTCSCGTDFCN
ANYSHLPPPGSPGTPGSQGPQAAPGESIWMAL

A nucleic acid sequence encoding the MISRII precursor protein is shown below (SEQ ID NO: 52), corresponding to nucleotides 81-1799 of Genbank Reference Sequence NM_020547.2. The signal sequence is underlined.

(SEQ ID NO: 52)
ATGCTAGGGTCTTTGGGGCTTTGGGCATTACTTCCCACAGCTGTGGAAGC
ACCCCCAAACAGGCGAACCTGTGTGTTCTTTGAGGCCCCTGGAGTGCGGG
GAAGCACAAAGACACTGGGAGAGCTGCTAGATACAGGCACAGAGCTCCCC
AGAGCTATCCGCTGCCTCTACAGCCGCTGCTGCTTTGGGATCTGGAACCT
GACCCAAGACCGGGCACAGGTGGAAATGCAAGGATGCCGAGACAGTGATG
AGCCAGGCTGTGAGTCCCTCCACTGTGACCCAAGTCCCCGAGCCCACCCC
AGCCCTGGCTCCACTCTCTTCACCTGCTCCTGTGGCACTGACTTCTGCAA
TGCCAATTACAGCCATCTGCCTCCTCCAGGGAGCCCTGGGACTCCTGGCT
CCCAGGGTCCCCAGGCTGCCCCAGGTGAGTCCATCTGGATGGCACTGGTG
CTGCTGGGGCTGTTCCTCCTCCTCCTGCTGCTGCTGGGCAGCATCATCTT
GGCCCTGCTACAGCGAAAGAACTACAGAGTGCGAGGTGAGCCAGTGCCAG
AGCCAAGGCCAGACTCAGGCAGGGACTGGAGTGTGGAGCTGCAGGAGCTG
CCTGAGCTGTGTTTCTCCCAGGTAATCCGGGAAGGAGGTCATGCAGTGGT
TTGGGCCGGGCAGCTGCAAGGAAAACTGGTTGCCATCAAGGCCTTCCCAC
CGAGGTCTGTGGCTCAGTTCCAAGCTGAGAGAGCATTGTACGAACTTCCA
GGCCTACAGCACGACCACATTGTCCGATTTATCACTGCCAGCCGGGGGGG
TCCTGGCCGCCTGCTCTCTGGGCCCCTGCTGGTACTGGAACTGCATCCCA
AGGGCTCCCTGTGCCACTACTTGACCCAGTACACCAGTGACTGGGGAAGT
TCCCTGCGGATGGCACTGTCCCTGGCCCAGGGCCTGGCATTTCTCCATGA
GGAGCGCTGGCAGAATGGCCAATATAAACCAGGTATTGCCCACCGAGATC
TGAGCAGCCAGAATGTGCTCATTCGGGAAGATGGATCGTGTGCCATTGGA
GACCTGGGCCTTGCCTTGGTGCTCCCTGGCCTCACTCAGCCCCCTGCCTG
GACCCCTACTCAACCACAAGGCCCAGCTGCCATCATGGAAGCTGGCACCC
AGAGGTACATGGCACCAGAGCTCTTGGACAAGACTCTGGACCTACAGGAT
TGGGGCATGGCCCTCCGACGAGCTGATATTTACTCTTTGGCTCTGCTCCT
GTGGGAGATACTGAGCCGCTGCCCAGATTTGAGGCCTGACAGCAGTCCAC
CACCCTTCCAACTGGCCTATGAGGCAGAACTGGGCAATACCCCTACCTCT
GATGAGCTATGGGCCTTGGCAGTGCAGGAGAGGAGGCGTCCCTACATCCC
ATCCACCTGGCGCTGCTTTGCCACAGACCCTGATGGGCTGAGGGAGCTCC
TAGAAGACTGTTGGGATGCAGACCCAGAAGCACGGCTGACAGCTGAGTGT
GTACAGCAGCGCCTGGCTGCCTTGGCCCATCCTCAAGAGAGCCACCCCTT
TCCAGAGAGCTGTCCACGTGGCTGCCCACCTCTCTGCCCAGAAGACTGTA
CTTCAATTCCTGCCCCTACCATCCTCCCCTGTAGGCCTCAGCGGAGTGCC
TGCCACTTCAGCGTTCAGCAAGGCCCTTGTTCCAGGAATCCTCAGCCTGC
CTGTACCCTTTCTCCTGTG

A nucleic acid sequence encoding the extracellular human MISRII polypeptide is as follows:

(SEQ ID NO: 53)
CCCCCAAACAGGCGAACCTGTGTGTTCTTTGAGGCCCCTGGAGTGCGGGG
AAGCACAAAGACACTGGGAGAGCTGCTAGATACAGGCACAGAGCTCCCCA
GAGCTATCCGCTGCCTCTACAGCCGCTGCTGCTTTGGGATCTGGAACCTG
ACCCAAGACCGGGCACAGGTGGAAATGCAAGGATGCCGAGACAGTGATGA
GCCAGGCTGTGAGTCCCTCCACTGTGACCCAAGTCCCCGAGCCCACCCCA
GCCCTGGCTCCACTCTCTTCACCTGCTCCTGTGGCACTGACTTCTGCAAT
GCCAATTACAGCCATCTGCCTCCTCCAGGGAGCCCTGGGACTCCTGGCTC
CCAGGGTCCCCAGGCTGCCCCAGGTGAGTCCATCTGGATGGCACTG

An alternative isoform of the human MISRII precursor protein sequence, isoform 2 (NCBI Ref Seq NP_001158162.1), is as follows:

| (SEQ ID NO: 75) | |
|---|---|
| 1 | MLGSLGLWAL LPTAVEAPPN RRTCVFFEAP GVRGSTKTLG ELLDTGTELP |
| 051 | RAIRCLYSRC CFGIWNLTQD RAQVEMQGCR DSDEPGCESL HCDPSPRAHP |
| 101 | SPGSTLFTCS CGTDFCNANY SHLPPPGSPG TPGSQGPQAA PGESIWMALV |
| 151 | LLGLFLLLLL LLGSIILALL QRKNYRVRGE PVPEPRPDSG RDWSVELQEL |
| 201 | PELCFSQVIR EGGHAVVWAG QLQGKLVAIK AFPPRSVAQF QAERALYELP |
| 251 | GLQHDHIVRF ITASRGGPGR LLSGPLLVLE LHPKGSLCHY LTQYTSDWGS |
| 301 | SLRMALSLAQ GLAFLHEERW QNGQYKPGIA HRDLSSQNVL IREDGSCAIG |
| 351 | DLGLALVLPG LTQPPAWTPT QPQGPAAIME AGTQRYMAPE LLDKTLDLQD |
| 401 | WGMALRRADI YSLALLLWEI LSRCPDLRPA VHHPSNWPMR QNWAIPLPLM |
| 451 | SYGPWQCRRG GVPTSHPPGA ALPQTLMG |

The signal peptide is indicated by a single underline and the extracellular domain is indicated in bold font.

The processed extracellular MISRII polypeptide sequence (isoform 2) is as follows:

(SEQ ID NO: 76)
PPNRRTCVFFEAPGVRGSTKTLGELLDTGTELPRAIRCLYSRCCFGIWNL
TQDRAQVEMQGCRDSDEPGCESLHCDPSPRAHPSPGSTLFTCSCGTDFCN
ANYSHLPPPGSPGTPGSQGPQAAPGESIWMAL

A nucleic acid sequence encoding the MISRII precursor protein (isoform 2) is shown below (SEQ ID NO: 77), corresponding to nucleotides 81-1514 of Genbank Reference Sequence NM_001164690.1. The signal sequence is underlined.

(SEQ ID NO: 77)
ATGCTAGGGTCTTTGGGGCTTTGGGCATTACTTCCCACAGCTGTGGAAGC
ACCCCCAAACAGGCGAACCTGTGTGTTCTTTGAGGCCCCTGGAGTGCGGG
GAAGCACAAAGACACTGGGAGAGCTGCTAGATACAGGCACAGAGCTCCCC
AGAGCTATCCGCTGCCTCTACAGCCGCTGCTGCTTTGGGATCTGGAACCT
GACCCAAGACCGGGCACAGGTGGAAATGCAAGGATGCCGAGACAGTGATG
AGCCAGGCTGTGAGTCCCTCCACTGTGACCCAAGTCCCCGAGCCCACCCC
AGCCCTGGCTCCACTCTCTTCACCTGCTCCTGTGGCACTGACTTCTGCAA
TGCCAATTACAGCCATCTGCCTCCTCCAGGGAGCCCTGGGACTCCTGGCT
CCCAGGGTCCCCAGGCTGCCCCAGGTGAGTCCATCTGGATGGCACTGGTG
CTGCTGGGGCTGTTCCTCCTCCTCCTGCTGCTGCTGGGCAGCATCATCTT
GGCCCTGCTACAGCGAAAGAACTACAGAGTGCGAGGTGAGCCAGTGCCAG
AGCCAAGGCCAGACTCAGGCAGGGACTGGAGTGTGGAGCTGCAGGAGCTG
CCTGAGCTGTGTTTCTCCCAGGTAATCCGGGAAGGAGGTCATGCAGTGGT
TTGGGCCGGGCAGCTGCAAGGAAAACTGGTTGCCATCAAGGCCTTCCCAC
CGAGGTCTGTGGCTCAGTTCCAAGCTGAGAGAGCATTGTACGAACTTCCA
GGCCTACAGCACGACCACATTGTCCGATTTATCACTGCCAGCCGGGGGGG
TCCTGGCCGCCTGCTCTCTGGGCCCCTGCTGGTACTGGAACTGCATCCCA
AGGGCTCCCTGTGCCACTACTTGACCCAGTACACCAGTGACTGGGGAAGT
TCCCTGCGGATGGCACTGTCCCTGGCCCAGGGCCTGGCATTTCTCCATGA
GGAGCGCTGGCAGAATGGCCAATATAAACCAGGTATTGCCCACCGAGATC
TGAGCAGCCAGAATGTGCTCATTCGGGAAGATGGATCGTGTGCCATTGGA
GACCTGGGCCTTGCCTTGGTGCTCCCTGGCCTCACTCAGCCCCCTGCCTG
GACCCCTACTCAACCACAAGGCCCAGCTGCCATCATGGAAGCTGGCACCC
AGAGGTACATGGCACCAGAGCTCTTGGACAAGACTCTGGACCTACAGGAT
TGGGGCATGGCCCTCCGACGAGCTGATATTTACTCTTTGGCTCTGCTCCT
GTGGGAGATACTGAGCCGCTGCCCAGATTTGAGGCCTGCAGTCCACCACC
CTTCCAACTGGCCTATGAGGCAGAACTGGGCAATACCCCTACCTCTGATG
AGCTATGGGCCTTGGCAGTGCAGGAGAGGAGGCGTCCCTACATCCCATCC
ACCTGGCGCTGCTTTGCCACAGACCCTGATGGGC

The nucleic acid sequence encoding processed soluble (extracellular) human MISRII polypeptide (isoform 2) is as follows:

(SEQ ID NO: 78)
CCCCCAAACAGGCGAACCTGTGTGTTCTTTGAGGCCCCTGGAGTGCGGGG
AAGCACAAAGACACTGGGAGAGCTGCTAGATACAGGCACAGAGCTCCCCA
GAGCTATCCGCTGCCTCTACAGCCGCTGCTGCTTTGGGATCTGGAACCTG
ACCCAAGACCGGGCACAGGTGGAAATGCAAGGATGCCGAGACAGTGATGA
GCCAGGCTGTGAGTCCCTCCACTGTGACCCAAGTCCCCGAGCCCACCCCA
GCCCTGGCTCCACTCTCTTCACCTGCTCCTGTGGCACTGACTTCTGCAAT
GCCAATTACAGCCATCTGCCTCCTCCAGGGAGCCCTGGGACTCCTGGCTC
CCAGGGTCCCCAGGCTGCCCCAGGTGAGTCCATCTGGATGGCACTG

An alternative isoform of the human MISRII precursor protein sequence, isoform 3 (NCBI Ref Seq NP_001158163.1), is as follows:

| (SEQ ID NO: 79) | |
|---|---|
| 1 | MLGSLGLWAL LPTAVEAPPN RRTCVFFEAP GVRGSTKTLG ELLDTGTELP |
| 51 | RAIRCLYSRC CFGIWNLTQD RAQVEMQGCR DSDEPGCESL HCDPSPRAHP |
| 101 | SPGSTLFTCS CGTDFCNANY SHLPPPGSPG TPGSQGPQAA PGESIWMALV |
| 151 | LLGLFLLLLL LLGSIILALL QRKNYRVRGE PVPEPRPDSG RDWSVELQEL |
| 201 | PELCFSQVIR EGGHAVVWAG QLQGKLVAIK AFPPRSVAQF QAERALYELP |
| 251 | GLQHDHIVRF ITASRGGPGR LLSGPLLVLE LHPKGSLCHY LTQYTSDWGS |
| 301 | SLRMALSLAQ GLAFLHEERW QNGQYKPGIA HRDLSSQNVL IREDGSCAIG |
| 351 | DLGLALVLPG LTQPPAWTPT QPQGPAAIME DPDGLRELLE DCWDADPEAR |
| 401 | LTAECVQQRL AALAHPQESH PFPESCPRGC PPLCPEDCTS IPAPTILPCR |
| 451 | PQRSACHFSV QQGPCSRNPQ PACTLSPV |

The signal peptide is indicated by a single underline and the extracellular domain is indicated in bold font.

The processed extracellular MISRII polypeptide sequence (isoform 3) is as follows:

(SEQ ID NO: 80)
PPNRRTCVFFEAPGVRGSTKTLGELLDTGTELPRAIRCLYSRCCFGIWNL
TQDRAQVEMQGCRDSDEPGCESLHCDPSPRAHPSPGSTLFTCSCGTDFCN
ANYSHLPPPGSPGTPGSQGPQAAPGESIWMAL

A nucleic acid sequence encoding human MISRII precursor protein (isoform 3) is shown below (SEQ ID NO: 81), corresponding to nucleotides 81-1514 of Genbank Reference Sequence NM_001164691.1. The signal sequence is underlined.

(SEQ ID NO: 81)
ATGCTAGGGTCTTTGGGGCTTTGGGCATTACTTCCCACAGCTGTGGAAGC
ACCCCCAAACAGGCGAACCTGTGTGTTCTTTGAGGCCCCTGGAGTGCGGG
GAAGCACAAAGACACTGGGAGAGCTGCTAGATACAGGCACAGAGCTCCCC
AGAGCTATCCGCTGCCTCTACAGCCGCTGCTGCTTTGGGATCTGGAACCT
GACCCAAGACCGGGCACAGGTGGAAATGCAAGGATGCCGAGACAGTGATG
AGCCAGGCTGTGAGTCCCTCCACTGTGACCCAAGTCCCCGAGCCCACCCC
AGCCCTGGCTCCACTCTCTTCACCTGCTCCTGTGGCACTGACTTCTGCAA
TGCCAATTACAGCCATCTGCCTCCTCCAGGGAGCCCTGGGACTCCTGGCT
CCCAGGGTCCCCAGGCTGCCCCAGGTGAGTCCATCTGGATGGCACTGGTG
CTGCTGGGGCTGTTCCTCCTCCTCCTGCTGCTGCTGGGCAGCATCATCTT
GGCCCTGCTACAGCGAAAGAACTACAGAGTGCGAGGTGAGCCAGTGCCAG
AGCCAAGGCCAGACTCAGGCAGGGACTGGAGTGTGGAGCTGCAGGAGCTG
CCTGAGCTGTGTTTCTCCCAGGTAATCCGGGAAGGAGGTCATGCAGTGGT
TTGGGCCGGGCAGCTGCAAGGAAAACTGGTTGCCATCAAGGCCTTCCCAC
CGAGGTCTGTGGCTCAGTTCCAAGCTGAGAGAGCATTGTACGAACTTCCA
GGCCTACAGCACGACCACATTGTCCGATTTATCACTGCCAGCCGGGGGGG
TCCTGGCCGCCTGCTCTCTGGGCCCCTGCTGGTACTGGAACTGCATCCCA
AGGGCTCCCTGTGCCACTACTTGACCCAGTACACCAGTGACTGGGGAAGT
TCCCTGCGGATGGCACTGTCCCTGGCCCAGGGCCTGGCATTTCTCCATGA
GGAGCGCTGGCAGAATGGCCAATATAAACCAGGTATTGCCCACCGAGATC
TGAGCAGCCAGAATGTGCTCATTCGGGAAGATGGATCGTGTGCCATTGGA
GACCTGGGCCTTGCCTTGGTGCTCCCTGGCCTCACTCAGCCCCCTGCCTG
GACCCCTACTCAACCACAAGGCCCAGCTGCCATCATGGAAGACCCTGATG
GGCTGAGGGAGCTCCTAGAAGACTGTTGGGATGCAGACCCAGAAGCACGG
CTGACAGCTGAGTGTGTACAGCAGCGCCTGGCTGCCTTGGCCCATCCTCA
AGAGAGCCACCCCTTTCCAGAGAGCTGTCCACGTGGCTGCCCACCTCTCT
GCCCAGAAGACTGTACTTCAATTCCTGCCCCTACCATCCTCCCCTGTAGG
CCTCAGCGGAGTGCCTGCCACTTCAGCGTTCAGCAAGGCCCTTGTTCCAG
GAATCCTCAGCCTGCCTGTACCCTTTCTCCTGTG

A nucleic acid sequence encoding processed soluble (extracellular) human MISRII polypeptide (isoform 3) is as follows:

(SEQ ID NO: 82)
CCCCCAAACAGGCGAACCTGTGTGTTCTTTGAGGCCCCTGGAGTGCGGGG
AAGCACAAAGACACTGGGAGAGCTGCTAGATACAGGCACAGAGCTCCCCA
GAGCTATCCGCTGCCTCTACAGCCGCTGCTGCTTTGGGATCTGGAACCTG
ACCCAAGACCGGGCACAGGTGGAAATGCAAGGATGCCGAGACAGTGATGA
GCCAGGCTGTGAGTCCCTCCACTGTGACCCAAGTCCCCGAGCCCACCCCA
GCCCTGGCTCCACTCTCTTCACCTGCTCCTGTGGCACTGACTTCTGCAAT
GCCAATTACAGCCATCTGCCTCCTCCAGGGAGCCCTGGGACTCCTGGCTC
CCAGGGTCCCCAGGCTGCCCCAGGTGAGTCCATCTGGATGGCACTG

In certain embodiments, the disclosure relates to heteromultimer complexes that comprise at least one MISRII polypeptide, which includes fragments, functional variants, and modified forms thereof. Preferably, MISRII polypeptides for use in accordance with inventions of the disclosure (e.g., heteromultimer complexes comprising a MISRII polypeptide and uses thereof) are soluble (e.g., an extracellular domain of MISRII). In other preferred embodiments, MISRII polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one MISRII polypeptide that is at least 70%, 75%, 80%, 85%, 90%, 95%, 97%, 98%, or 99% identical to the amino acid sequence of SEQ ID NOs: 50, 51, 75, 76, 79, or 80. In some embodiments, heteromultimer complexes of the disclosure consist or consist essentially of at least one MISRII polypeptide that is at least 70%, 75%, 80%, 85%, 90%, 95%, 97%, 98%, or 99% identical to the amino acid sequence of SEQ ID NOs: 50, 51, 75, 76, 79, or 80.

In certain aspects, the present disclosure relates to protein complexes that comprise an ALK1 polypeptide. As used herein, the term “ALK1” refers to a family of activin receptor-like kinase-1 proteins from any species and variants derived from such ALK1 proteins by mutagenesis or other modification. Reference to ALK1 herein is understood to be a reference to any one of the currently identified forms. Members of the ALK1 family are generally transmembrane proteins, composed of a ligand-binding extracellular domain with a cysteine-rich region, a transmembrane domain, and a cytoplasmic domain with predicted serine/threonine kinase activity.

The term “ALK1 polypeptide” includes polypeptides comprising any naturally occurring polypeptide of an ALK1 family member as well as any variants thereof (including mutants, fragments, fusions, and peptidomimetic forms) that retain a useful activity. Numbering of amino acids for all ALK1-related polypeptides described herein is based on the numbering of the human ALK1 precursor protein sequence below (SEQ ID NO: 14), unless specifically designated otherwise.

The human ALK1 precursor protein sequence (NCBI Ref Seq NP_000011.2) is as follows:

| (SEQ ID NO: 14) | |
|---|---|
| 1 | MTLGSPRKGL LMLLMALVTQ GDPVKPSRGP LVTCTCESPH CKGPTCRGAW |
| 51 | CTVVLVREEG RHPQEHRGCG NLHRELCRGR PTEFVNHYCC DSHLCNHNVS |
| 101 | LVLEATQPPS EQPGTDGQLA LILGPVLALL ALVALGVLGL WHVRRRQEKQ |
| 151 | RGLHSELGES SLILKASEQG DSMLGDLLDS DCTTGSGSGL PFLVQRTVAR |
| 201 | QVALVECVGK GRYGEVWRGL WHGESVAVKI FSSRDEQSWF RETEIYNTVL |
| 251 | LRHDNILGFI ASDMTSRNSS TQLWLITHYH EHGSLYDFLQ RQTLEPHLAL |
| 301 | RLAVSAACGL AHLHVEIFGT QGKPAIAHRD FKSRNVLVKS NLQCCIADLG |
| 351 | LAVMHSQGSD YLDIGNNPRV GTKRYMAPEV LDEQIRTDCF ESYKWTDIWA |
| 401 | FGLVLWEIAR RTIVNGIVED YRPPFYDVVP NDPSFEDMKK VVCVDQQTPT |
| 451 | IPNRLAADPV LSGLAQMMRE CWYPNPSARL TALRIKKTLQ KISNSPEKPK |
| 501 | VIQ |

The signal peptide is indicated by a single underline and the extracellular domain is indicated in bold font.

The processed extracelluar ALK1 polypeptide sequence is as follows:

(SEQ ID NO: 15)
DPVKPSRGPLVTCTCESPHCKGPTCRGAWCTVVLVREEGRHPQEHRGCGN
LHRELCRGRPTEFVNHYCCDSHLCNHNVSLVLEATQPPSEQPGTDGQ

A nucleic acid sequence encoding human ALK1 precursor protein is shown below (SEQ ID NO: 16), corresponding to nucleotides 284-1792 of Genbank Reference Sequence NM_000020.2. The signal sequence is underlined.

(SEQ ID NO: 16)
ATGACCTTGGGCTCCCCCAGGAAAGGCCTTCTGATGCTGCTGATGGCCTT
GGTGACCCAGGGAGACCCTGTGAAGCCGTCTCGGGGCCCGCTGGTGACCT
GCACGTGTGAGAGCCCACATTGCAAGGGGCCTACCTGCCGGGGGGCCTGG
TGCACAGTAGTGCTGGTGCGGGAGGAGGGGAGGCACCCCCAGGAACATCG
GGGCTGCGGGAACTTGCACAGGGAGCTCTGCAGGGGGCGCCCCACCGAGT
TCGTCAACCACTACTGCTGCGACAGCCACCTCTGCAACCACAACGTGTCC
CTGGTGCTGGAGGCCACCCAACCTCCTTCGGAGCAGCCGGGAACAGATGG
CCAGCTGGCCCTGATCCTGGGCCCCGTGCTGGCCTTGCTGGCCCTGGTGG
CCCTGGGTGTCCTGGGCCTGTGGCATGTCCGACGGAGGCAGGAGAAGCAG
CGTGGCCTGCACAGCGAGCTGGGAGAGTCCAGTCTCATCCTGAAAGCATC
TGAGCAGGGCGACAGCATGTTGGGGGACCTCCTGGACAGTGACTGCACCA
CAGGGAGTGGCTCAGGGCTCCCCTTCCTGGTGCAGAGGACAGTGGCACGG
CAGGTTGCCTTGGTGGAGTGTGTGGGAAAAGGCCGCTATGGCGAAGTGTG
GCGGGGCTTGTGGCACGGTGAGAGTGTGGCCGTCAAGATCTTCTCCTCGA
GGGATGAACAGTCCTGGTTCCGGGAGACTGAGATCTATAACACAGTGTTG
CTCAGACACGACAACATCCTAGGCTTCATCGCCTCAGACATGACCTCCCG
CAACTCGAGCACGCAGCTGTGGCTCATCACGCACTACCACGAGCACGGCT
CCCTCTACGACTTTCTGCAGAGACAGACGCTGGAGCCCCATCTGGCTCTG
AGGCTAGCTGTGTCCGCGGCATGCGGCCTGGCGCACCTGCACGTGGAGAT
CTTCGGTACACAGGGCAAACCAGCCATTGCCCACCGCGACTTCAAGAGCC
GCAATGTGCTGGTCAAGAGCAACCTGCAGTGTTGCATCGCCGACCTGGGC
CTGGCTGTGATGCACTCACAGGGCAGCGATTACCTGGACATCGGCAACAA
CCCGAGAGTGGGCACCAAGCGGTACATGGCACCCGAGGTGCTGGACGAGC
AGATCCGCACGGACTGCTTTGAGTCCTACAAGTGGACTGACATCTGGGCC
TTTGGCCTGGTGCTGTGGGAGATTGCCCGCCGGACCATCGTGAATGGCAT
CGTGGAGGACTATAGACCACCCTTCTATGATGTGGTGCCCAATGACCCCA
GCTTTGAGGACATGAAGAAGGTGGTGTGTGTGGATCAGCAGACCCCCACC
ATCCCTAACCGGCTGGCTGCAGACCCGGTCCTCTCAGGCCTAGCTCAGAT
GATGCGGGAGTGCTGGTACCCAAACCCCTCTGCCCGACTCACCGCGCTGC
GGATCAAGAAGACACTACAAAAAATTAGCAACAGTCCAGAGAAGCCTAAA
GTGATTCAA

A nucleic acid sequence encoding processed extracelluar ALK1 polypeptide is as follows:

(SEQ ID NO: 17)
GACCCTGTGAAGCCGTCTCGGGGCCCGCTGGTGACCTGCACGTGTGAGAG
CCCACATTGCAAGGGGCCTACCTGCCGGGGGGCCTGGTGCACAGTAGTGC
TGGTGCGGGAGGAGGGGAGGCACCCCCAGGAACATCGGGGCTGCGGGAAC
TTGCACAGGGAGCTCTGCAGGGGGCGCCCCACCGAGTTCGTCAACCACTA
CTGCTGCGACAGCCACCTCTGCAACCACAACGTGTCCCTGGTGCTGGAGG
CCACCCAACCTCCTTCGGAGCAGCCGGGAACAGATGGCCAG

In certain embodiments, the disclosure relates to heteromultimer complexes that comprise at least one ALK1 polypeptide, which includes fragments, functional variants, and modified forms thereof. Preferably, ALK1 polypeptides for use in accordance with inventions of the disclosure (e.g., heteromultimer complexes comprising an ALK1 polypeptide and uses thereof) are soluble (e.g., an extracellular domain of ALK1). In other preferred embodiments, ALK1 polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one ALK1 polypeptide that is at least 70%, 75%, 80%, 85%, 90%, 95%, 97%, 98%, or 99% identical to the amino acid sequence of SEQ ID NO: 14, 15, 124, 126, 413, or 414. In some embodiments, heteromultimer complexes of the disclosure consist or consist essentially of at least one ALK1 polypeptide that is at least 70%, 75%, 80%, 85%, 90%, 95%, 97%, 98%, or 99% identical to the amino acid sequence of SEQ ID NO: 14, 15, 124, 126, 413, or 414.

In certain aspects, the present disclosure relates to protein complexes that comprise an ALK2 polypeptide. As used herein, the term “ALK2” refers to a family of activin receptor-like kinase-2 proteins from any species and variants derived from such ALK2 proteins by mutagenesis or other modification. Reference to ALK2 herein is understood to be a reference to any one of the currently identified forms. Members of the ALK2 family are generally transmembrane proteins, composed of a ligand-binding extracellular domain with a cysteine-rich region, a transmembrane domain, and a cytoplasmic domain with predicted serine/threonine kinase activity.

The term “ALK2 polypeptide” includes polypeptides comprising any naturally occurring polypeptide of an ALK2 family member as well as any variants thereof (including mutants, fragments, fusions, and peptidomimetic forms) that retain a useful activity. Numbering of amino acids for all ALK2-related polypeptides described herein is based on the numbering of the human ALK2 precursor protein sequence below (SEQ ID NO: 18), unless specifically designated otherwise.

The human ALK2 precursor protein sequence (NCBI Ref Seq NP_001096.1) is as follows:

| (SEQ ID NO: 18) | |
|---|---|
| 1 | MVDGVMILPV LIMIALPSPS MEDEKPKVNP KLYMCVCEGL SCGNEDHCEG |
| 51 | QQCFSSLSIN DGFHVYQKGC FQVYEQGKMT CKTPPSPGQA VECCQGDWCN |
| 101 | RNITAQLPTK GKSFPGTQNF HLEVGLIILS VVFAVCLLAC LLGVALRKFK |
| 151 | RRNQERLNPR DVEYGTIEGL ITTNVGDSTL ADLLDHSCTS GSGSGLPFLV |
| 201 | QRTVARQITL LECVGKGRYG EVWRGSWQGE NVAVKIFSSR DEKSWFRETE |
| 251 | LYNTVMLRHE NILGFIASDM TSRHSSTQLW LITHYHEMGS LYDYLQLTTL |
| 301 | DTVSCLRIVL SIASGLAHLH IEIFGTQGKP AIAHRDLKSK NILVKKNGQC |
| 351 | CIADLGLAVM HSQSTNQLDV GNNPRVGTKR YMAPEVLDET IQVDCFDSYK |
| 401 | RVDIWAFGLV LWEVARRMVS NGIVEDYKPP FYDVVPNDPS FEDMRKVVCV |
| 451 | DQQRPNIPNR WFSDPTLTSL AKLMKECWYQ NPSARLTALR IKKTLTKIDN |
| 501 | SLDKLKTDC |

The signal peptide is indicated by a single underline and the extracellular domain is indicated in bold font.

The processed extracellular ALK2 polypeptide sequence is as follows:

(SEQ ID NO: 19)
MEDEKPKVNPKLYMCVCEGLSCGNEDHCEGQQCFSSLSINDGFHVYQKGC
FQVYEQGKMTCKTPPSPGQAVECCQGDWCNRNITAQLPTKGKSFPGTQNF
HLE

A nucleic acid sequence encoding human ALK2 precursor protein is shown below (SEQ ID NO: 20), corresponding to nucleotides 431-1957 of Genbank Reference Sequence NM_001105.4. The signal sequence is underlined.

(SEQ ID NO: 20)
ATGGTAGATGGAGTGATGATTCTTCCTGTGCTTATCATGATTGCTCTCCC
CTCCCCTAGTATGGAAGATGAGAAGCCCAAGGTCAACCCCAAACTCTACA
TGTGTGTGTGTGAAGGTCTCTCCTGCGGTAATGAGGACCACTGTGAAGGC
CAGCAGTGCTTTTCCTCACTGAGCATCAACGATGGCTTCCACGTCTACCA
GAAAGGCTGCTTCCAGGTTTATGAGCAGGGAAAGATGACCTGTAAGACCC
CGCCGTCCCCTGGCCAAGCCGTGGAGTGCTGCCAAGGGGACTGGTGTAAC
AGGAACATCACGGCCCAGCTGCCCACTAAAGGAAAATCCTTCCCTGGAAC
ACAGAATTTCCACTTGGAGGTTGGCCTCATTATTCTCTCTGTAGTGTTCG
CAGTATGTCTTTTAGCCTGCCTGCTGGGAGTTGCTCTCCGAAAATTTAAA
AGGCGCAACCAAGAACGCCTCAATCCCCGAGACGTGGAGTATGGCACTAT
CGAAGGGCTCATCACCACCAATGTTGGAGACAGCACTTTAGCAGATTTAT
TGGATCATTCGTGTACATCAGGAAGTGGCTCTGGTCTTCCTTTTCTGGTA
CAAAGAACAGTGGCTCGCCAGATTACACTGTTGGAGTGTGTCGGGAAAGG
CAGGTATGGTGAGGTGTGGAGGGGCAGCTGGCAAGGGGAGAATGTTGCCG
TGAAGATCTTCTCCTCCCGTGATGAGAAGTCATGGTTCAGGGAAACGGAA
TTGTACAACACTGTGATGCTGAGGCATGAAAATATCTTAGGTTTCATTGC
TTCAGACATGACATCAAGACACTCCAGTACCCAGCTGTGGTTAATTACAC
ATTATCATGAAATGGGATCGTTGTACGACTATCTTCAGCTTACTACTCTG
GATACAGTTAGCTGCCTTCGAATAGTGCTGTCCATAGCTAGTGGTCTTGC
ACATTTGCACATAGAGATATTTGGGACCCAAGGGAAACCAGCCATTGCCC
ATCGAGATTTAAAGAGCAAAAATATTCTGGTTAAGAAGAATGGACAGTGT
TGCATAGCAGATTTGGGCCTGGCAGTCATGCATTCCCAGAGCACCAATCA
GCTTGATGTGGGGAACAATCCCCGTGTGGGCACCAAGCGCTACATGGCCC
CCGAAGTTCTAGATGAAACCATCCAGGTGGATTGTTTCGATTCTTATAAA
AGGGTCGATATTTGGGCCTTTGGACTTGTTTTGTGGGAAGTGGCCAGGCG
GATGGTGAGCAATGGTATAGTGGAGGATTACAAGCCACCGTTCTACGATG
TGGTTCCCAATGACCCAAGTTTTGAAGATATGAGGAAGGTAGTCTGTGTG
GATCAACAAAGGCCAAACATACCCAACAGATGGTTCTCAGACCCGACATT
AACCTCTCTGGCCAAGCTAATGAAAGAATGCTGGTATCAAAATCCATCCG
CAAGACTCACAGCACTGCGTATCAAAAAGACTTTGACCAAAATTGATAAT
TCCCTCGACAAATTGAAAACTGACTGT

A nucleic acid sequence encoding the extracellular ALK2 polypeptide is as follows:

(SEQ ID NO: 21)
ATGGAAGATGAGAAGCCCAAGGTCAACCCCAAACTCTACATGTGTGTGTG
TGAAGGTCTCTCCTGCGGTAATGAGGACCACTGTGAAGGCCAGCAGTGCT
TTTCCTCACTGAGCATCAACGATGGCTTCCACGTCTACCAGAAAGGCTGC
TTCCAGGTTTATGAGCAGGGAAAGATGACCTGTAAGACCCCGCCGTCCCC
TGGCCAAGCCGTGGAGTGCTGCCAAGGGGACTGGTGTAACAGGAACATCA
CGGCCCAGCTGCCCACTAAAGGAAAATCCTTCCCTGGAACACAGAATTTC
CACTTGGAG

In certain embodiments, the disclosure relates to heteromultimer complexes that comprise at least one ALK2 polypeptide, which includes fragments, functional variants, and modified forms thereof. Preferably, ALK2 polypeptides for use in accordance with inventions of the disclosure (e.g., heteromultimer complexes comprising an ALK2 polypeptide and uses thereof) are soluble (e.g., an extracellular domain of ALK2). In other preferred embodiments, ALK2 polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one ALK2 polypeptide that is at least 70%, 75%, 80%, 85%, 90%, 95%, 97%, 98%, or 99% identical to the amino acid sequence of SEQ ID NO: 18 or 19. In some embodiments, heteromultimer complexes of the disclosure consist or consist essentially of at least one ALK2 polypeptide that is at least 70%, 75%, 80%, 85%, 90%, 95%, 97%, 98%, or 99% identical to the amino acid sequence of SEQ ID NO: 18 or 19.

In certain aspects, the present disclosure relates to protein complexes that comprise an ALK3 polypeptide. As used herein, the term “ALK3” refers to a family of activin receptor-like kinase-3 proteins from any species and variants derived from such ALK3 proteins by mutagenesis or other modification. Reference to ALK3 herein is understood to be a reference to any one of the currently identified forms. Members of the ALK3 family are generally transmembrane proteins, composed of a ligand-binding extracellular domain with a cysteine-rich region, a transmembrane domain, and a cytoplasmic domain with predicted serine/threonine kinase activity.

The term “ALK3 polypeptide” includes polypeptides comprising any naturally occurring polypeptide of an ALK3 family member as well as any variants thereof (including mutants, fragments, fusions, and peptidomimetic forms) that retain a useful activity. Numbering of amino acids for all ALK3-related polypeptides described herein is based on the numbering of the human ALK3 precursor protein sequence below (SEQ ID NO: 22), unless specifically designated otherwise.

The human ALK3 precursor protein sequence (NCBI Ref Seq NP_004320.2) is as follows:

| (SEQ ID NO: 22) | |
|---|---|
| 1 | MPQLYIYIRL LGAYLFIISR VQGQNLDSML HGTGMKSDSD QKKSENGVTL APEDTLPFLK |
| 61 | CYCSGHCPDD AINNTCITNG HCFAIIEEDD QGETTLASGC MKYEGSDFQC KDSPKAQLRR |
| 121 | TIECCRTNLC NQYLQPTLPP VVIGPFFDGS IRWLVLLISM AVCIIAMIIF SSCFCYKHYC |
| 181 | KSISSRRRYN RDLEQDEAFI PVGESLKDLI DQSQSSGSGS GLPLLVQRTI AKQIQMVRQV |
| 241 | GKGRYGEVWM GKWRGEKVAV KVFFTTEEAS WFRETEIYQT VLMRHENILG FIAADIKGTG |
| 301 | SWTQLYLITD YHENGSLYDF LKCATLDTRA LLKLAYSAAC GLCHLHTEIY GTQGKPAIAH |
| 361 | RDLKSKNILI KKNGSCCIAD LGLAVKFNSD TNEVDVPLNT RVGTKRYMAP EVLDESLNKN |
| 421 | HFQPYIMADI YSFGLIIWEM ARRCITGGIV EEYQLPYYNM VPSDPSYEDM REVVCVKRLR |
| 481 | PIVSNRWNSD ECLRAVLKLM SECWAHNPAS RLTALRIKKT LAKMVESQDV KI |

The signal peptide is indicated by a single underline and the extracellular domain is indicated in bold font.

The processed extracellular ALK3 polypeptide sequence is as follows:

(SEQ ID NO: 23)

| | |
|---|---|
| 1 | QNLDSMLHGT GMKSDSDQKK SENGVTLAPE DTLPFLKCYC |
| | SGHCPDDAIN NTCITNGHCF |
| 61 | AIIEEDDQGE TTLASGCMKY EGSDFQCKDS PKAQLRRTIE |
| | CCRTNLCNQY LQPTLPPVVI |
| 121 | GPFFDGSIR |

A nucleic acid sequence encoding human ALK3 precursor protein is shown below (SEQ ID NO: 24), corresponding to nucleotides 549-2144 of Genbank Reference Sequence NM_004329.2. The signal sequence is underlined and the extracellular domain is indicated in bold font.

| (SEQ ID NO: 24) | |
|---|---|
| 1 | ATGCCTCAGC TATACATTTA CATCAGATTA TTGGGAGCCT ATTTGTTCAT CATTTCTCGT |
| 61 | GTTCAAGGAC AGAATCTGGA TAGTATGCTT CATGGCACTG GGATGAAATC AGACTCCGAC |
| 121 | CAGAAAAAGT CAGAAAATGG AGTAACCTTA GCACCAGAGG ATACCTTGCC TTTTTTAAAG |
| 181 | TGCTATTGCT CAGGGCACTG TCCAGATGAT GCTATTAATA ACACATGCAT AACTAATGGA |
| 241 | CATTGCTTTG CCATCATAGA AGAAGATGAC CAGGGAGAAA CCACATTAGC TTCAGGGTGT |
| 301 | ATGAAATATG AAGGATCTGA TTTTCAGTGC AAAGATTCTC CAAAAGCCCA GCTACGCCGG |
| 361 | ACAATAGAAT GTTGTCGGAC CAATTTATGT AACCAGTATT TGCAACCCAC ACTGCCCCCT |
| 421 | GTTGTCATAG GTCCGTTTTT TGATGGCAGC ATTCGATGGC TGGTTTTGCT CATTTCTATG |
| 481 | GCTGTCTGCA TAATTGCTAT GATCATCTTC TCCAGCTGCT TTTGTTACAA ACATTATTGC |
| 541 | AAGAGCATCT CAAGCAGACG TCGTTACAAT CGTGATTTGG AACAGGATGA AGCATTTATT |
| 601 | CCAGTTGGAG AATCACTAAA AGACCTTATT GACCAGTCAC AAAGTTCTGG TAGTGGGTCT |
| 661 | GGACTACCTT TATTGGTTCA GCGAACTATT GCCAAACAGA TTCAGATGGT CCGGCAAGTT |
| 721 | GGTAAAGGCC GATATGGAGA AGTATGGATG GGCAAATGGC GTGGCGAAAA AGTGGCGGTG |
| 781 | AAAGTATTCT TTACCACTGA AGAAGCCAGC TGGTTTCGAG AAACAGAAAT CTACCAAACT |
| 841 | GTGCTAATGC GCCATGAAAA CATACTTGGT TTCATAGCGG CAGACATTAA AGGTACAGGT |
| 901 | TCCTGGACTC AGCTCTATTT GATTACTGAT TACCATGAAA ATGGATCTCT CTATGACTTC |
| 961 | CTGAAATGTG CTACACTGGA CACCAGAGCC CTGCTTAAAT TGGCTTATTC AGCTGCCTGT |
| 1021 | GGTCTGTGCC ACCTGCACAC AGAAATTTAT GGCACCCAAG GAAAGCCCGC AATTGCTCAT |
| 1081 | CGAGACCTAA AGAGCAAAAA CATCCTCATC AAGAAAAATG GGAGTTGCTG CATTGCTGAC |
| 1141 | CTGGGCCTTG CTGTTAAATT CAACAGTGAC ACAAATGAAG TTGATGTGCC CTTGAATACC |
| 1201 | AGGGTGGGCA CCAAACGCTA CATGGCTCCC GAAGTGCTGG ACGAAAGCCT GAACAAAAAC |
| 1261 | CACTTCCAGC CCTACATCAT GGCTGACATC TACAGCTTCG GCCTAATCAT TTGGGAGATG |
| 1321 | GCTCGTCGTT GTATCACAGG AGGGATCGTG GAAGAATACC AATTGCCATA TTACAACATG |
| 1381 | GTACCGAGTG ATCCGTCATA CGAAGATATG CGTGAGGTTG TGTGTGTCAA ACGTTTGCGG |
| 1441 | CCAATTGTGT CTAATCGGTG GAACAGTGAT GAATGTCTAC GAGCAGTTTT GAAGCTAATG |
| 1501 | TCAGAATGCT GGGCCCACAA TCCAGCCTCC AGACTCACAG CATTGAGAAT TAAGAAGACG |
| 1561 | CTTGCCAAGA TGGTTGAATC CCAAGATGTA AAAATC |

A nucleic acid sequence encoding the extracelluar human ALK3 polypeptide is as follows:

| (SEQ ID NO: 25) | |
|---|---|
| 1 | CAGAATCTGG ATAGTATGCT TCATGGCACT GGGATGAAAT CAGACTCCGA CCAGAAAAAG |
| 61 | TCAGAAAATG GAGTAACCTT AGCACCAGAG GATACCTTGC CTTTTTTAAA GTGCTATTGC |
| 121 | TCAGGGCACT GTCCAGATGA TGCTATTAAT AACACATGCA TAACTAATGG ACATTGCTTT |
| 181 | GCCATCATAG AAGAAGATGA CCAGGGAGAA ACCACATTAG CTTCAGGGTG TATGAAATAT |
| 241 | GAAGGATCTG ATTTTCAGTG CAAAGATTCT CCAAAAGCCC AGCTACGCCG GACAATAGAA |
| 301 | TGTTGTCGGA CCAATTTATG TAACCAGTAT TTGCAACCCA CACTGCCCCC TGTTGTCATA |
| 361 | GGTCCGTTTT TTGATGGCAG CATTCGA |

A general formula for an active (e.g., ligand binding) ALK3 polypeptide is one that comprises a polypeptide that begins at any amino acid position 25-31 (i.e., position 25, 26, 27, 28, 29, 30, or 31) of SEQ ID NO: 22 and ends at any amino acid position 140-152 of SEQ ID NO: 22 (i.e., 140, 141, 142, 143, 144, 145, 146, 147, 148, 149, 150, 151, or 152). See U.S. Pat. No. 8,338,377, the teachings of which are incorporated herein by reference in their entirety.

In certain embodiments, the disclosure relates to heteromultimer complexes that comprise at least one ALK3 polypeptide, which includes fragments, functional variants, and modified forms thereof. Preferably, ALK3 polypeptides for use in accordance with inventions of the disclosure (e.g., heteromultimer complexes comprising an ALK3 polypeptide and uses thereof) are soluble (e.g., an extracellular domain of ALK3). In other preferred embodiments, ALK3 polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one ALK3 polypeptide that comprises, consists, or consists essentially of an amino acid beginning at any amino acid position 25-31 (i.e., position 25, 26, 27, 28, 29, 30, or 31) of SEQ ID NO: 22 and ending at any amino acid position 140-153 of SEQ ID NO: 22 (i.e., 140, 141, 142, 143, 144, 145, 146, 147, 148, 149, 150, 151, or 152) of SEQ ID NO: 22. In some embodiments, heteromultimer complexes of the disclosure comprise at least one ALK3 polypeptide that is at least 70%, 75%, 80%, 85%, 90%, 95%, 97%, 98%, or 99% identical to the amino acid sequence of SEQ ID NO: 22, 23, 115, 117, 407, or 408. In some embodiments, heteromultimer complexes of the disclosure consist or consist essentially of at least one ALK3 polypeptide that is at least 70%, 75%, 80%, 85%, 90%, 95%, 97%, 98%, or 99% identical to the amino acid sequence of SEQ ID NO: 22, 23, 115, 117, 407, or 408.

In certain aspects, the present disclosure relates to protein complexes that comprise an ALK4 polypeptide. As used herein, the term “ALK4” refers to a family of activin receptor-like kinase-4 proteins from any species and variants derived from such ALK4 proteins by mutagenesis or other modification. Reference to ALK4 herein is understood to be a reference to any one of the currently identified forms. Members of the ALK4 family are generally transmembrane proteins, composed of a ligand-binding extracellular domain with a cysteine-rich region, a transmembrane domain, and a cytoplasmic domain with predicted serine/threonine kinase activity.

The term “ALK4 polypeptide” includes polypeptides comprising any naturally occurring polypeptide of an ALK4 family member as well as any variants thereof (including mutants, fragments, fusions, and peptidomimetic forms) that retain a useful activity. Numbering of amino acids for all ALK4-related polypeptides described herein is based on the numbering of the human ALK4 precursor protein sequence below (SEQ ID NO: 26), unless specifically designated otherwise.

The canonical human ALK4 precursor protein sequence (NCBI Ref Seq NP_004293) is as follows:

| (SEQ ID NO: 26) | |
|---|---|
| 1 | MAESAGASSF FPLVVLLLAG SGGSGPRGVQ ALLCACTSCL QANYTCETDG ACMVSIFNLD |
| 61 | GMEHHVRTCI PKVELVPAGK PFYCLSSEDL RNTHCCYTDY CNRIDLRVPS GHLKEPEHPS |
| 121 | MWGPVELVGI IAGPVFLLFL IIIIVFLVIN YHQRVYHNRQ RLDMEDPSCE MCLSKDKTLQ |
| 181 | DLVYDLSTSG SGSGLPLFVQ RTVARTIVLQ EIIGKGRFGE VWRGRWRGGD VAVKIFSSRE |
| 241 | ERSWFREAEI YQTVMLRHEN ILGFIAADNK DNGTWTQLWL VSDYHEHGSL FDYLNRYTVT |
| 301 | IEGMIKLALS AASGLAHLHM EIVGTQGKPG IAHRDLKSKN ILVKKNGMCA IADLGLAVRH |
| 361 | DAVTDTIDIA PNQRVGTKRY MAPEVLDETI NMKHFDSFKC ADIYALGLVY WEIARRCNSG |
| 421 | GVHEEYQLPY YDLVPSDPSI EEMRKVVCDQ KLRPNIPNWW QSYEALRVMG KMMRECWYAN |
| 481 | GAARLTALRI KKTLSQLSVQ EDVKI |

The signal peptide is indicated by a single underline and the extracellular domain is indicated in bold font.

The processed extracellular human ALK4 polypeptide sequence is as follows:

(SEQ ID NO: 27)
SGPRGVQALLCACTSCLQANYTCETDGACMVSIFNLDGMEHHVRTCIPKV
ELVPAGKPFYCLSSEDLRNTHCCYTDYCNRIDLRVPSGHLKEPEHPSMWG
PVE

A nucleic acid sequence encoding the ALK4 precursor protein is shown below (SEQ ID NO: 28), corresponding to nucleotides 78-1592 of Genbank Reference Sequence NM_004302.4. The signal sequence is underlined and the extracellular domain is indicated in bold font.

(SEQ ID NO: 28)
ATGGCGGAGTCGGCCGGAGCCTCCTCCTTCTTCCCCCTTGTTGTCCTCCT
GCTCGCCGGCAGCGGCGGGTCCGGGCCCCGGGGGGTCCAGGCTCTGCTGT
GTGCGTGCACCAGCTGCCTCCAGGCCAACTACACGTGTGAGACAGATGGG
GCCTGCATGGTTTCCATTTTCAATCTGGATGGGATGGAGCACCATGTGCG
CACCTGCATCCCCAAAGTGGAGCTGGTCCCTGCCGGGAAGCCCTTCTACT
GCCTGAGCTCGGAGGACCTGCGCAACACCCACTGCTGCTACACTGACTAC
TGCAACAGGATCGACTTGAGGGTGCCCAGTGGTCACCTCAAGGAGCCTGA
GCACCCGTCCATGTGGGGCCCGGTGGAGCTGGTAGGCATCATCGCCGGCC
CGGTGTTCCTCCTGTTCCTCATCATCATCATTGTTTTCCTTGTCATTAAC
TATCATCAGCGTGTCTATCACAACCGCCAGAGACTGGACATGGAAGATCC
CTCATGTGAGATGTGTCTCTCCAAAGACAAGACGCTCCAGGATCTTGTCT
ACGATCTCTCCACCTCAGGGTCTGGCTCAGGGTTACCCCTCTTTGTCCAG
CGCACAGTGGCCCGAACCATCGTTTTACAAGAGATTATTGGCAAGGGTCG
GTTTGGGGAAGTATGGCGGGGCCGCTGGAGGGGTGGTGATGTGGCTGTGA
AAATATTCTCTTCTCGTGAAGAACGGTCTTGGTTCAGGGAAGCAGAGATA
TACCAGACGGTCATGCTGCGCCATGAAAACATCCTTGGATTTATTGCTGC
TGACAATAAAGATAATGGCACCTGGACACAGCTGTGGCTTGTTTCTGACT
ATCATGAGCACGGGTCCCTGTTTGATTATCTGAACCGGTACACAGTGACA
ATTGAGGGGATGATTAAGCTGGCCTTGTCTGCTGCTAGTGGGCTGGCACA
CCTGCACATGGAGATCGTGGGCACCCAAGGGAAGCCTGGAATTGCTCATC
GAGACTTAAAGTCAAAGAACATTCTGGTGAAGAAAAATGGCATGTGTGCC
ATAGCAGACCTGGGCCTGGCTGTCCGTCATGATGCAGTCACTGACACCAT
TGACATTGCCCCGAATCAGAGGGTGGGGACCAAACGATACATGGCCCCTG
AAGTACTTGATGAAACCATTAATATGAAACACTTTGACTCCTTTAAATGT
GCTGATATTTATGCCCTCGGGCTTGTATATTGGGAGATTGCTCGAAGATG
CAATTCTGGAGGAGTCCATGAAGAATATCAGCTGCCATATTACGACTTAG
TGCCCTCTGACCCTTCCATTGAGGAAATGCGAAAGGTTGTATGTGATCAG
AAGCTGCGTCCCAACATCCCCAACTGGTGGCAGAGTTATGAGGCACTGCG
GGTGATGGGGAAGATGATGCGAGAGTGTTGGTATGCCAACGGCGCAGCCC
GCCTGACGGCCCTGCGCATCAAGAAGACCCTCTCCCAGCTCAGCGTGCAG
GAAGACGTGAAGATC

A nucleic acid sequence encoding the extracellular ALK4 polypeptide is as follows:

(SEQ ID NO: 29)
TCCGGGCCCCGGGGGGTCCAGGCTCTGCTGTGTGCGTGCACCAGCTGCCT
CCAGGCCAACTACACGTGTGAGACAGATGGGGCCTGCATGGTTTCCATTT
TCAATCTGGATGGGATGGAGCACCATGTGCGCACCTGCATCCCCAAAGTG
GAGCTGGTCCCTGCCGGGAAGCCCTTCTACTGCCTGAGCTCGGAGGACCT
GCGCAACACCCACTGCTGCTACACTGACTACTGCAACAGGATCGACTTGA
GGGTGCCCAGTGGTCACCTCAAGGAGCCTGAGCACCCGTCCATGTGGGGC
CCGGTGGAG

An alternative isoform of human ALK4 precursor protein sequence, isoform C (NCBI Ref Seq NP_064733.3), is as follows:

| (SEQ ID NO: 83) | |
|---|---|
| 1 | MAESAGASSF FPLVVLLLAG SGGSGPRGVQ ALLCACTSCL QANYTCETDG ACMVSIFNLD |
| 61 | GMEHHVRTCI PKVELVPAGK PFYCLSSEDL RNTHCCYTDY CNRIDLRVPS GHLKEPEHPS |
| 121 | MWGPVELVGI IAGPVFLLFL IIIIVFLVIN YHQRVYHNRQ RLDMEDPSCE MCLSKDKTLQ |
| 181 | DLVYDLSTSG SGSGLPLFVQ RTVARTIVLQ EIIGKGRFGE VWRGRWRGGD VAVKIFSSRE |
| 241 | ERSWFREAEI YQTVMLRHEN ILGFIAADNK ADCSFLTLPW EVVMVSAAPK LRSLRLQYKG |
| 301 | GRGRARFLFP LNNGTWTQLW LVSDYHEHGS LFDYLNRYTV TIEGMIKLAL SAASGLAHLH |
| 361 | MEIVGTQGKP GIAHRDLKSK NILVKKNGMC AIADLGLAVR HDAVTDTIDI APNQRVGTKR |
| 421 | YMAPEVLDET INMKHFDSFK CADIYALGLV YWEIARRCNS GGVHEEYQLP YYDLVPSDPS |
| 481 | IEEMRKVVCD QKLRPNIPNW WQSYEALRVM GKMMRECWYA NGAARLTALR IKKTLSQLSV |
| 541 | QEDVKI |

The signal peptide is indicated by a single underline and the extracellular domain is indicated in bold font.

The processed extracellular ALK4 polypeptide sequence (isoform C) is as follows:

(SEQ ID NO: 84)
SGPRGVQALLCACTSCLQANYTCETDGACMVSIFNLDGMEHHVRTCIPKV
ELVPAGKPFYCLSSEDLRNTHCCYTDYCNRIDLRVPSGHLKEPEHPSMWG
PVE

A nucleic acid sequence encoding the ALK4 precursor protein (isoform C) is shown below (SEQ ID NO: 85), corresponding to nucleotides 78-1715 of Genbank Reference Sequence NM_020328.3. The signal sequence is underlined and the extracellular domain is indicated in bold font.

(SEQ ID NO: 85)
ATGGCGGAGTCGGCCGGAGCCTCCTCCTTCTTCCCCCTTGTTGTCCTCCT
GCTCGCCGGCAGCGGCGGGTCCGGGCCCCGGGGGGTCCAGGCTCTGCTGT
GTGCGTGCACCAGCTGCCTCCAGGCCAACTACACGTGTGAGACAGATGGG
GCCTGCATGGTTTCCATTTTCAATCTGGATGGGATGGAGCACCATGTGCG
CACCTGCATCCCCAAAGTGGAGCTGGTCCCTGCCGGGAAGCCCTTCTACT
GCCTGAGCTCGGAGGACCTGCGCAACACCCACTGCTGCTACACTGACTAC
TGCAACAGGATCGACTTGAGGGTGCCCAGTGGTCACCTCAAGGAGCCTGA
GCACCCGTCCATGTGGGGCCCGGTGGAGCTGGTAGGCATCATCGCCGGCC
CGGTGTTCCTCCTGTTCCTCATCATCATCATTGTTTTCCTTGTCATTAAC
TATCATCAGCGTGTCTATCACAACCGCCAGAGACTGGACATGGAAGATCC
CTCATGTGAGATGTGTCTCTCCAAAGACAAGACGCTCCAGGATCTTGTCT
ACGATCTCTCCACCTCAGGGTCTGGCTCAGGGTTACCCCTCTTTGTCCAG
CGCACAGTGGCCCGAACCATCGTTTTACAAGAGATTATTGGCAAGGGTCG
GTTTGGGGAAGTATGGCGGGGCCGCTGGAGGGGTGGTGATGTGGCTGTGA
AAATATTCTCTTCTCGTGAAGAACGGTCTTGGTTCAGGGAAGCAGAGATA
TACCAGACGGTCATGCTGCGCCATGAAAACATCCTTGGATTTATTGCTGC
TGACAATAAAGCAGACTGCTCATTCCTCACATTGCCATGGGAAGTTGTAA
TGGTCTCTGCTGCCCCCAAGCTGAGGAGCCTTAGACTCCAATACAAGGGA
GGAAGGGGAAGAGCAAGATTTTTATTCCCACTGAATAATGGCACCTGGAC
ACAGCTGTGGCTTGTTTCTGACTATCATGAGCACGGGTCCCTGTTTGATT
ATCTGAACCGGTACACAGTGACAATTGAGGGGATGATTAAGCTGGCCTTG
TCTGCTGCTAGTGGGCTGGCACACCTGCACATGGAGATCGTGGGCACCCA
AGGGAAGCCTGGAATTGCTCATCGAGACTTAAAGTCAAAGAACATTCTGG
TGAAGAAAAATGGCATGTGTGCCATAGCAGACCTGGGCCTGGCTGTCCGT
CATGATGCAGTCACTGACACCATTGACATTGCCCCGAATCAGAGGGTGGG
GACCAAACGATACATGGCCCCTGAAGTACTTGATGAAACCATTAATATGA
AACACTTTGACTCCTTTAAATGTGCTGATATTTATGCCCTCGGGCTTGTA
TATTGGGAGATTGCTCGAAGATGCAATTCTGGAGGAGTCCATGAAGAATA
TCAGCTGCCATATTACGACTTAGTGCCCTCTGACCCTTCCATTGAGGAAA
TGCGAAAGGTTGTATGTGATCAGAAGCTGCGTCCCAACATCCCCAACTGG
TGGCAGAGTTATGAGGCACTGCGGGTGATGGGGAAGATGATGCGAGAGTG
TTGGTATGCCAACGGCGCAGCCCGCCTGACGGCCCTGCGCATCAAGAAGA
CCCTCTCCCAGCTCAGCGTGCAGGAAGACGTGAAGATC

A nucleic acid sequence encoding the extracelluar ALK4 polypeptide (isoform C) is as follows:

(SEQ ID NO: 86)
TCCGGGCCCCGGGGGGTCCAGGCTCTGCTGTGTGCGTGCACCAGCTGCCT
CCAGGCCAACTACACGTGTGAGACAGATGGGGCCTGCATGGTTTCCATTT
TCAATCTGGATGGGATGGAGCACCATGTGCGCACCTGCATCCCCAAAGTG
GAGCTGGTCCCTGCCGGGAAGCCCTTCTACTGCCTGAGCTCGGAGGACCT
GCGCAACACCCACTGCTGCTACACTGACTACTGCAACAGGATCGACTTGA
GGGTGCCCAGTGGTCACCTCAAGGAGCCTGAGCACCCGTCCATGTGGGGC
CCGGTGGAG

In certain embodiments, the disclosure relates to heteromultimer complexes that comprise at least one ALK4 polypeptide, which includes fragments, functional variants, and modified forms thereof. Preferably, ALK4 polypeptides for use in accordance with inventions of the disclosure (e.g., heteromultimer complexes comprising an ALK4 polypeptide and uses thereof) are soluble (e.g., an extracellular domain of ALK4). In other preferred embodiments, ALK4 polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one ALK4 polypeptide that is at least 70%, 75%, 80%, 85%, 90%, 95%, 97%, 98%, or 99% identical to the amino acid sequence of SEQ ID NO: 26, 27, 83, 84, 104, 106, 403, or 404. In some embodiments, heteromultimer complexes of the disclosure consist or consist essentially of at least one ALK4 polypeptide that is at least 70%, 75%, 80%, 85%, 90%, 95%, 97%, 98%, or 99% identical to the amino acid sequence of SEQ ID NO: 26, 27, 83, 84, 104, 106, 403, or 404.

In certain aspects, the present disclosure relates to protein complexes that comprise an ALK5 polypeptide. As used herein, the term “ALK5” refers to a family of activin receptor-like kinase-5 proteins from any species and variants derived from such ALK4 proteins by mutagenesis or other modification. Reference to ALK5 herein is understood to be a reference to any one of the currently identified forms. Members of the ALK5 family are generally transmembrane proteins, composed of a ligand-binding extracellular domain with a cysteine-rich region, a transmembrane domain, and a cytoplasmic domain with predicted serine/threonine kinase activity.

The term “ALK5 polypeptide” includes polypeptides comprising any naturally occurring polypeptide of an ALK5 family member as well as any variants thereof (including mutants, fragments, fusions, and peptidomimetic forms) that retain a useful activity. Numbering of amino acids for all ALK5-related polypeptides described herein is based on the numbering of the human ALK5 precursor protein sequence below (SEQ ID NO: 30), unless specifically designated otherwise.

The canonical human ALK5 precursor protein sequence (NCBI Ref Seq NP_004603.1) is as follows:

| (SEQ ID NO: 30) | |
|---|---|
| 1 | MEAAVAAPRP RLLLLVLAAA AAAAALLPG ATALQCFCHL CTKDNFTCVT DGLCFVSVTE |
| 61 | TTDKVIHNSM CIAEIDLIPR DRPFVCAPSS KTGSVTTTYC CNQDHCNKIE LPTTVKSSPG |
| 121 | LGPVELAAVI AGPVCFVCIS LMLMVYICHN RTVIHHRVPN EEDPSLDRPF ISEGTTLKDL |
| 181 | IYDMTTSGSG SGLPLLVQRT IARTIVLQES IGKGRFGEVW RGKWRGEEVA VKIFSSREER |
| 241 | SWFREAEIYQ TVMLRHENIL GFIAADNKDN GTWTQLWLVS DYHEHGSLFD YLNRYTVTVE |
| 301 | GMIKLALSTA SGLAHLHMEI VGTQGKPAIA HRDLKSKNIL VKKNGTCCIA DLGLAVRHDS |
| 361 | ATDTIDIAPN HRVGTKRYMA PEVLDDSINM KHFESFKRAD IYAMGLVFWE IARRCSIGGI |
| 421 | HEDYQLPYYD LVPSDPSVEE MRKVVCEQKL RPNIPNRWQS CEALRVMAKI MRECWYANGA |
| 481 | ARLTALRIKK TLSQLSQQEG IKM |

The signal peptide is indicated by a single underline and the extracellular domain is indicated in bold font.

The processed extracellular ALK5 polypeptide sequence is as follows:

(SEQ ID NO: 31)
AALLPGATALQCFCHLCTKDNFTCVTDGLCFVSVTETTDKVIHNSMCIAE
IDLIPRDRPFVCAPSSKTGSVTTTYCCNQDHCNKIELPTTVKSSPGLGPV
EL

A nucleic acid sequence encoding the ALK5 precursor protein is shown below (SEQ ID NO: 32), corresponding to nucleotides 77-1585 of Genbank Reference Sequence NM_004612.2. The signal sequence is underlined and the extracellular domain is indicated in bold font.

(SEQ ID NO: 32)
ATGGAGGCGGCGGTCGCTGCTCCGCGTCCCCGGCTGCTCCTCCTCGTGCT
GGCGGCGGCGGCGGCGGCGGCGGCGGCGCTGCTCCCGGGGGCGACGGCGT
TACAGTGTTTCTGCCACCTCTGTACAAAAGACAATTTTACTTGTGTGACA
GATGGGCTCTGCTTTGTCTCTGTCACAGAGACCACAGACAAAGTTATACA
CAACAGCATGTGTATAGCTGAAATTGACTTAATTCCTCGAGATAGGCCGT
TTGTATGTGCACCCTCTTCAAAAACTGGGTCTGTGACTACAACATATTGC
TGCAATCAGGACCATTGCAATAAAATAGAACTTCCAACTACTGTAAAGTC
ATCACCTGGCCTTGGTCCTGTGGAACTGGCAGCTGTCATTGCTGGACCAG
TGTGCTTCGTCTGCATCTCACTCATGTTGATGGTCTATATCTGCCACAAC
CGCACTGTCATTCACCATCGAGTGCCAAATGAAGAGGACCCTTCATTAGA
TCGCCCTTTTATTTCAGAGGGTACTACGTTGAAAGACTTAATTTATGATA
TGACAACGTCAGGTTCTGGCTCAGGTTTACCATTGCTTGTTCAGAGAACA
ATTGCGAGAACTATTGTGTTACAAGAAAGCATTGGCAAAGGTCGATTTGG
AGAAGTTTGGAGAGGAAAGTGGCGGGGAGAAGAAGTTGCTGTTAAGATAT
TCTCCTCTAGAGAAGAACGTTCGTGGTTCCGTGAGGCAGAGATTTATCAA
ACTGTAATGTTACGTCATGAAAACATCCTGGGATTTATAGCAGCAGACAA
TAAAGACAATGGTACTTGGACTCAGCTCTGGTTGGTGTCAGATTATCATG
AGCATGGATCCCTTTTTGATTACTTAAACAGATACACAGTTACTGTGGAA
GGAATGATAAAACTTGCTCTGTCCACGGCGAGCGGTCTTGCCCATCTTCA
CATGGAGATTGTTGGTACCCAAGGAAAGCCAGCCATTGCTCATAGAGATT
TGAAATCAAAGAATATCTTGGTAAAGAAGAATGGAACTTGCTGTATTGCA
GACTTAGGACTGGCAGTAAGACATGATTCAGCCACAGATACCATTGATAT
TGCTCCAAACCACAGAGTGGGAACAAAAAGGTACATGGCCCCTGAAGTTC
TCGATGATTCCATAAATATGAAACATTTTGAATCCTTCAAACGTGCTGAC
ATCTATGCAATGGGCTTAGTATTCTGGGAAATTGCTCGACGATGTTCCAT
TGGTGGAATTCATGAAGATTACCAACTGCCTTATTATGATCTTGTACCTT
CTGACCCATCAGTTGAAGAAATGAGAAAAGTTGTTTGTGAACAGAAGTTA
AGGCCAAATATCCCAAACAGATGGCAGAGCTGTGAAGCCTTGAGAGTAAT
GGCTAAAATTATGAGAGAATGTTGGTATGCCAATGGAGCAGCTAGGCTTA
CAGCATTGCGGATTAAGAAAACATTATCGCAACTCAGTCAACAGGAAGGC
ATCAAAATG

A nucleic acid sequence encoding the extracellular human ALK5 polypeptide is as follows:

(SEQ ID NO: 33)
GCGGCGCTGCTCCCGGGGGCGACGGCGTTACAGTGTTTCTGCCACCTCTG
TACAAAAGACAATTTTACTTGTGTGACAGATGGGCTCTGCTTTGTCTCTG
TCACAGAGACCACAGACAAAGTTATACACAACAGCATGTGTATAGCTGAA
ATTGACTTAATTCCTCGAGATAGGCCGTTTGTATGTGCACCCTCTTCAAA
AACTGGGTCTGTGACTACAACATATTGCTGCAATCAGGACCATTGCAATA
AAATAGAACTTCCAACTACTGTAAAGTCATCACCTGGCCTTGGTCCTGTG
GAACTG

An alternative isoform of the human ALK5 precursor protein sequence, isoform 2 (NCBI Ref Seq XP_005252207.1), is as follows:

| (SEQ ID NO: 87) | |
|---|---|
| 1 | MEAAVAAPRP RLLLLVLAAA AAAAAALLPG ATALQCFCHL CTKDNFTCVT DGLCFVSVTE |
| 61 | TTDKVIHNSM CIAEIDLIPR DRPFVCAPSS KTGSVTTTYC CNQDHCNKIE LPTTGPFSVK |
| 121 | SSPGLGPVEL AAVIAGPVCF VCISLMLMVY ICHNRTVIHH RVPNEEDPSL DRPFISEGTT |
| 181 | LKDLIYDMTT SGSGSGLPLL VQRTIARTIV LQESIGKGRF GEVWRGKWRG EEVAVKIFSS |
| 241 | REERSWFREA EIYQTVMLRH ENILGFIAAD NKDNGTWTQL WLVSDYHEHG SLFDYLNRYT |
| 301 | VTVEGMIKLA LSTASGLAHL HMEIVGTQGK PAIAHRDLKS KNILVKKNGT CCIADLGLAV |
| 361 | RHDSATDTID IAPNHRVGTK RYMAPEVLDD SINMKHFESF KRADIYAMGL VFWEIARRCS |
| 421 | IGGIHEDYQL PYYDLVPSDP SVEEMRKVVC EQKLRPNIPN RWQSCEALRV MAKIMRECWY |
| 481 | ANGAARLTAL RIKKTLSQLS QQEGIKM |

The signal peptide is indicated by a single underline and the extracellular domain is indicated in bold font.

The processed extracellular ALK5 polypeptide sequence (isoform 2) is as follows:

(SEQ ID NO: 88)
AALLPGATALQCFCHLCTKDNFTCVTDGLCFVSVTETTDKVIHNSMCIAE
IDLIPRDRPFVCAPSSKTGSVTTTYCCNQDHCNKIELPTTGPFSVKSSPG
LGPVEL

A nucleic acid sequence encoding human ALK5 precursor protein (isoform 2) is shown below (SEQ ID NO: 89), corresponding to nucleotides 77-1597 of Genbank Reference Sequence XM_005252150.1. The signal sequence is underlined and the extracellular domain is indicated in bold font.

(SEQ ID NO: 89)
ATGGAGGCGGCGGTCGCTGCTCCGCGTCCCCGGCTGCTCCTCCTCGTGCT
GGCGGCGGCGGCGGCGGCGGCGGCGGCGCTGCTCCCGGGGGCGACGGCGT
TACAGTGTTTCTGCCACCTCTGTACAAAAGACAATTTTACTTGTGTGACA
GATGGGCTCTGCTTTGTCTCTGTCACAGAGACCACAGACAAAGTTATACA
CAACAGCATGTGTATAGCTGAAATTGACTTAATTCCTCGAGATAGGCCGT
TTGTATGTGCACCCTCTTCAAAAACTGGGTCTGTGACTACAACATATTGC
TGCAATCAGGACCATTGCAATAAAATAGAACTTCCAACTACTGGCCCTTT
TTCAGTAAAGTCATCACCTGGCCTTGGTCCTGTGGAACTGGCAGCTGTCA
TTGCTGGACCAGTGTGCTTCGTCTGCATCTCACTCATGTTGATGGTCTAT
ATCTGCCACAACCGCACTGTCATTCACCATCGAGTGCCAAATGAAGAGGA
CCCTTCATTAGATCGCCCTTTTATTTCAGAGGGTACTACGTTGAAAGACT
TAATTTATGATATGACAACGTCAGGTTCTGGCTCAGGTTTACCATTGCTT
GTTCAGAGAACAATTGCGAGAACTATTGTGTTACAAGAAAGCATTGGCAA
AGGTCGATTTGGAGAAGTTTGGAGAGGAAAGTGGCGGGGAGAAGAAGTTG
CTGTTAAGATATTCTCCTCTAGAGAAGAACGTTCGTGGTTCCGTGAGGCA
GAGATTTATCAAACTGTAATGTTACGTCATGAAAACATCCTGGGATTTAT
AGCAGCAGACAATAAAGACAATGGTACTTGGACTCAGCTCTGGTTGGTGT
CAGATTATCATGAGCATGGATCCCTTTTTGATTACTTAAACAGATACACA
GTTACTGTGGAAGGAATGATAAAACTTGCTCTGTCCACGGCGAGCGGTCT
TGCCCATCTTCACATGGAGATTGTTGGTACCCAAGGAAAGCCAGCCATTG
CTCATAGAGATTTGAAATCAAAGAATATCTTGGTAAAGAAGAATGGAACT
TGCTGTATTGCAGACTTAGGACTGGCAGTAAGACATGATTCAGCCACAGA
TACCATTGATATTGCTCCAAACCACAGAGTGGGAACAAAAAGGTACATGG
CCCCTGAAGTTCTCGATGATTCCATAAATATGAAACATTTTGAATCCTTC
AAACGTGCTGACATCTATGCAATGGGCTTAGTATTCTGGGAAATTGCTCG
ACGATGTTCCATTGGTGGAATTCATGAAGATTACCAACTGCCTTATTATG
ATCTTGTACCTTCTGACCCATCAGTTGAAGAAATGAGAAAAGTTGTTTGT
GAACAGAAGTTAAGGCCAAATATCCCAAACAGATGGCAGAGCTGTGAAGC
CTTGAGAGTAATGGCTAAAATTATGAGAGAATGTTGGTATGCCAATGGAG
CAGCTAGGCTTACAGCATTGCGGATTAAGAAAACATTATCGCAACTCAGT
CAACAGGAAGGCATCAAAATG

A nucleic acid sequence encoding the processed extracellular ALK5 polypeptide is as follows:

(SEQ ID NO: 90)
GCGGCGCTGCTCCCGGGGGCGACGGCGTTACAGTGTTTCTGCCACCTCTG
TACAAAAGACAATTTTACTTGTGTGACAGATGGGCTCTGCTTTGTCTCTG
TCACAGAGACCACAGACAAAGTTATACACAACAGCATGTGTATAGCTGAA
ATTGACTTAATTCCTCGAGATAGGCCGTTTGTATGTGCACCCTCTTCAAA
AACTGGGTCTGTGACTACAACATATTGCTGCAATCAGGACCATTGCAATA
AAATAGAACTTCCAACTACTGGCCCTTTTTCAGTAAAGTCATCACCTGGC
CTTGGTCCTGTGGAACTG

In certain embodiments, the disclosure relates to heteromultimer complexes that comprise at least one ALK5 polypeptide, which includes fragments, functional variants, and modified forms thereof. Preferably, ALK5 polypeptides for use in accordance with inventions of the disclosure (e.g., heteromultimer complexes comprising an ALK5 polypeptide and uses thereof) are soluble (e.g., an extracellular domain of ALK5). In other preferred embodiments, ALK5 polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one ALK5 polypeptide that is at least 70%, 75%, 80%, 85%, 90%, 95%, 97%, 98%, or 99% identical to the amino acid sequence of SEQ ID NO: 30, 31, 87, or 88. In some embodiments, heteromultimer complexes of the disclosure consist or consist essentially of at least one ALK5 polypeptide that is at least 70%, 75%, 80%, 85%, 90%, 95%, 97%, 98%, or 99% identical to the amino acid sequence of SEQ ID NO: 30, 31, 87, or 88.

In certain aspects, the present disclosure relates to protein complexes that comprise an ALK6 polypeptide. As used herein, the term “ALK6” refers to a family of activin receptor-like kinase-6 proteins from any species and variants derived from such ALK6 proteins by mutagenesis or other modification. Reference to ALK6 herein is understood to be a reference to any one of the currently identified forms. Members of the ALK6 family are generally transmembrane proteins, composed of a ligand-binding extracellular domain with a cysteine-rich region, a transmembrane domain, and a cytoplasmic domain with predicted serine/threonine kinase activity.

The term “ALK6 polypeptide” includes polypeptides comprising any naturally occurring polypeptide of an ALK6 family member as well as any variants thereof (including mutants, fragments, fusions, and peptidomimetic forms) that retain a useful activity. Numbering of amino acids for all ALK6-related polypeptides described herein is based on the numbering of the human ALK6 precursor protein sequence below (SEQ ID NO: 34), unless specifically designated otherwise.

The canonical human ALK6 precursor protein sequence (NCBI Ref Seq NP_001194.1) is as follows:

| (SEQ ID NO: 34) | |
|---|---|
| 1 | MLLRSAGKLN VGTKKEDGES TAPTPRPKVL RCKCHHHCPE DSVNNICSTD GYCFTMIEED |
| 61 | DSGLPVVTSG CLGLEGSDFQ CRDTPIPHQR RSIECCTERN ECNKDLHPTL PPLKNRDFVD |
| 121 | GPIHHRALLI SVTVCSLLLV LIILFCYFRY KRQETRPRYS IGLEQDETYI PPGESLRDLI |
| 181 | EQSQSSGSGS GLPLLVQRTI AKQIQMVKQI GKGRYGEVWM GKWRGEKVAV KVFFTTEEAS |
| 241 | WFRETEIYQT VLMRHENILG FIAADIKGTG SWTQLYLITD YHENGSLYDY LKSTTLDAKS |
| 301 | MLKLAYSSVS GLCHLHTEIF STQGKPAIAH RDLKSKNILV KKNGTCCIAD LGLAVKFISD |
| 361 | TNEVDIPPNT RVGTKRYMPP EVLDESLNRN HFQSYIMADM YSFGLILWEV ARRCVSGGIV |
| 421 | EEYQLPYHDL VPSDPSYEDM REIVCIKKLR PSFPNRWSSD ECLRQMGKLM TECWAHNPAS |
| 481 | RLTALRVKKT LAKMSESQDI KL |

The signal peptide is indicated by a single underline and the extracellular domain is indicated in bold font.

The processed extracellular ALK6 polypeptide sequence is as follows:

(SEQ ID NO: 35)
KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPL
KNRDFVDGPIHHR

A nucleic acid sequence encoding the ALK6 precursor protein is shown below (SEQ ID NO: 36), corresponding to nucleotides 275-1780 of Genbank Reference Sequence NM_001203.2. The signal sequence is underlined and the extracellular domain is indicated in bold font.

(SEQ ID NO: 36)
ATGCTTTTGCGAAGTGCAGGAAAATTAAATGTGGGCACCAAGAAAGAGGA
TGGTGAGAGTACAGCCCCCACCCCCCGTCCAAAGGTCTTGCGTTGTAAAT
GCCACCACCATTGTCCAGAAGACTCAGTCAACAATATTTGCAGCACAGAC
GGATATTGTTTCACGATGATAGAAGAGGATGACTCTGGGTTGCCTGTGGT
CACTTCTGGTTGCCTAGGACTAGAAGGCTCAGATTTTCAGTGTCGGGACA
CTCCCATTCCTCATCAAAGAAGATCAATTGAATGCTGCACAGAAAGGAAC
GAATGTAATAAAGACCTACACCCTACACTGCCTCCATTGAAAAACAGAGA
TTTTGTTGATGGACCTATACACCACAGGGCTTTACTTATATCTGTGACTG
TCTGTAGTTTGCTCTTGGTCCTTATCATATTATTTTGTTACTTCCGGTAT
AAAAGACAAGAAACCAGACCTCGATACAGCATTGGGTTAGAACAGGATGA
AACTTACATTCCTCCTGGAGAATCCCTGAGAGACTTAATTGAGCAGTCTC
AGAGCTCAGGAAGTGGATCAGGCCTCCCTCTGCTGGTCCAAAGGACTATA
GCTAAGCAGATTCAGATGGTGAAACAGATTGGAAAAGGTCGCTATGGGGA
AGTTTGGATGGGAAAGTGGCGTGGCGAAAAGGTAGCTGTGAAAGTGTTCT
TCACCACAGAGGAAGCCAGCTGGTTCAGAGAGACAGAAATATATCAGACA
GTGTTGATGAGGCATGAAAACATTTTGGGTTTCATTGCTGCAGATATCAA
AGGGACAGGGTCCTGGACCCAGTTGTACCTAATCACAGACTATCATGAAA
ATGGTTCCCTTTATGATTATCTGAAGTCCACCACCCTAGACGCTAAATCA
ATGCTGAAGTTAGCCTACTCTTCTGTCAGTGGCTTATGTCATTTACACAC
AGAAATCTTTAGTACTCAAGGCAAACCAGCAATTGCCCATCGAGATCTGA
AAAGTAAAAACATTCTGGTGAAGAAAAATGGAACTTGCTGTATTGCTGAC
CTGGGCCTGGCTGTTAAATTTATTAGTGATACAAATGAAGTTGACATACC
ACCTAACACTCGAGTTGGCACCAAACGCTATATGCCTCCAGAAGTGTTGG
ACGAGAGCTTGAACAGAAATCACTTCCAGTCTTACATCATGGCTGACATG
TATAGTTTTGGCCTCATCCTTTGGGAGGTTGCTAGGAGATGTGTATCAGG
AGGTATAGTGGAAGAATACCAGCTTCCTTATCATGACCTAGTGCCCAGTG
ACCCCTCTTATGAGGACATGAGGGAGATTGTGTGCATCAAGAAGTTACGC
CCCTCATTCCCAAACCGGTGGAGCAGTGATGAGTGTCTAAGGCAGATGGG
AAAACTCATGACAGAATGCTGGGCTCACAATCCTGCATCAAGGCTGACAG
CCCTGCGGGTTAAGAAAACACTTGCCAAAATGTCAGAGTCCCAGGACATT
AAACTC

A nucleic acid sequence encoding processed extracellular ALK6 polypeptide is as follows:

(SEQ ID NO: 37)
AAGAAAGAGGATGGTGAGAGTACAGCCCCCACCCCCCGTCCAAAGGTCTT
GCGTTGTAAATGCCACCACCATTGTCCAGAAGACTCAGTCAACAATATTT
GCAGCACAGACGGATATTGTTTCACGATGATAGAAGAGGATGACTCTGGG
TTGCCTGTGGTCACTTCTGGTTGCCTAGGACTAGAAGGCTCAGATTTTCA
GTGTCGGGACACTCCCATTCCTCATCAAAGAAGATCAATTGAATGCTGCA
CAGAAAGGAACGAATGTAATAAAGACCTACACCCTACACTGCCTCCATTG
AAAAACAGAGATTTTGTTGATGGACCTATACACCACAGG

An alternative isoform of human ALK6 precursor protein sequence, isoform 2 (NCBI Ref Seq NP_001243722.1) is as follows:

| (SEQ ID NO: 91) | |
|---|---|
| 1 | MGWLEELNWQ LHIFLLILLS MHTRANFLDN MLLRSAGKLN VGTKKEDGES TAPTPRPKVL |
| 61 | RCKCHHHCPE DSVNNICSTD GYCFTMIEED DSGLPVVTSG CLGLEGSDFQ CRDTPIPHQR |
| 121 | RSIECCTERN ECNKDLHPTL PPLKNRDFVD GPIHHRALLI SVTVCSLLLV LIILFCYFRY |
| 181 | KRQETRPRYS IGLEQDETYI PPGESLRDLI EQSQSSGSGS GLPLLVQRTI AKQIQMVKQI |
| 241 | GKGRYGEVWM GKWRGEKVAV KVFFTTEEAS WFRETEIYQT VLMRHENILG FIAADIKGTG |
| 301 | SWTQLYLITD YHENGSLYDY LKSTTLDAKS MLKLAYSSVS GLCHLHTEIF STQGKPAIAH |
| 361 | RDLKSKNILV KKNGTCCIAD LGLAVKFISD TNEVDIPPNT RVGTKRYMPP EVLDESLNRN |
| 421 | HFQSYIMADM YSFGLILWEV ARRCVSGGIV EEYQLPYHDL VPSDPSYEDM REIVCIKKLR |
| 481 | PSFPNRWSSD ECLRQMGKLM TECWAHNPAS RLTALRVKKT LAKMSESQDI KL |

The signal peptide is indicated by a single underline and the extracellular domain is indicated in bold font.

The processed extracellular ALK6 polypeptide sequence (isoform 2) is as follows:

(SEQ ID NO: 92)
NFLDNMLLRSAGKLNVGTKKEDGESTAPTPRPKVLRCKCHHHCPEDSVNN
ICSTDGYCFTMIEEDDSGLPVVTSGCLGLEGSDFQCRDTPIPHQRRSIEC
CTERNECNKDLHPTLPPLKNRDFVDGPIHHR

A nucleic acid sequence encoding human ALK6 precursor protein (isoform 2) is shown below, corresponding to nucleotides 22-1617 of Genbank Reference Sequence NM_001256793.1. The signal sequence is underlined and the extracellular domain is indicated in bold font.

(SEQ ID NO: 93)
ATGGGTTGGCTGGAAGAACTAAACTGGCAGCTTCACATTTTCTTGCTCAT
TCTTCTCTCTATGCACACAAGGGCAAACTTCCTTGATAACATGCTTTTGC
GAAGTGCAGGAAAATTAAATGTGGGCACCAAGAAAGAGGATGGTGAGAGT
ACAGCCCCCACCCCCCGTCCAAAGGTCTTGCGTTGTAAATGCCACCACCA
TTGTCCAGAAGACTCAGTCAACAATATTTGCAGCACAGACGGATATTGTT
TCACGATGATAGAAGAGGATGACTCTGGGTTGCCTGTGGTCACTTCTGGT
TGCCTAGGACTAGAAGGCTCAGATTTTCAGTGTCGGGACACTCCCATTCC
TCATCAAAGAAGATCAATTGAATGCTGCACAGAAAGGAACGAATGTAATA
AAGACCTACACCCTACACTGCCTCCATTGAAAAACAGAGATTTTGTTGAT
GGACCTATACACCACAGGGCTTTACTTATATCTGTGACTGTCTGTAGTTT
GCTCTTGGTCCTTATCATATTATTTTGTTACTTCCGGTATAAAAGACAAG
AAACCAGACCTCGATACAGCATTGGGTTAGAACAGGATGAAACTTACATT
CCTCCTGGAGAATCCCTGAGAGACTTAATTGAGCAGTCTCAGAGCTCAGG
AAGTGGATCAGGCCTCCCTCTGCTGGTCCAAAGGACTATAGCTAAGCAGA
TTCAGATGGTGAAACAGATTGGAAAAGGTCGCTATGGGGAAGTTTGGATG
GGAAAGTGGCGTGGCGAAAAGGTAGCTGTGAAAGTGTTCTTCACCACAGA
GGAAGCCAGCTGGTTCAGAGAGACAGAAATATATCAGACAGTGTTGATGA
GGCATGAAAACATTTTGGGTTTCATTGCTGCAGATATCAAAGGGACAGGG
TCCTGGACCCAGTTGTACCTAATCACAGACTATCATGAAAATGGTTCCCT
TTATGATTATCTGAAGTCCACCACCCTAGACGCTAAATCAATGCTGAAGT
TAGCCTACTCTTCTGTCAGTGGCTTATGTCATTTACACACAGAAATCTTT
AGTACTCAAGGCAAACCAGCAATTGCCCATCGAGATCTGAAAAGTAAAAA
CATTCTGGTGAAGAAAAATGGAACTTGCTGTATTGCTGACCTGGGCCTGG
CTGTTAAATTTATTAGTGATACAAATGAAGTTGACATACCACCTAACACT
CGAGTTGGCACCAAACGCTATATGCCTCCAGAAGTGTTGGACGAGAGCTT
GAACAGAAATCACTTCCAGTCTTACATCATGGCTGACATGTATAGTTTTG
GCCTCATCCTTTGGGAGGTTGCTAGGAGATGTGTATCAGGAGGTATAGTG
GAAGAATACCAGCTTCCTTATCATGACCTAGTGCCCAGTGACCCCTCTTA
TGAGGACATGAGGGAGATTGTGTGCATCAAGAAGTTACGCCCCTCATTCC
CAAACCGGTGGAGCAGTGATGAGTGTCTAAGGCAGATGGGAAAACTCATG
ACAGAATGCTGGGCTCACAATCCTGCATCAAGGCTGACAGCCCTGCGGGT
TAAGAAAACACTTGCCAAAATGTCAGAGTCCCAGGACATTAAACTC

A nucleic acid sequence encoding the processed extracellular ALK6 polypeptide is as follows:

(SEQ ID NO: 94)
AACTTCCTTGATAACATGCTTTTGCGAAGTGCAGGAAAATTAAATGTGGG
CACCAAGAAAGAGGATGGTGAGAGTACAGCCCCCACCCCCCGTCCAAAGG
TCTTGCGTTGTAAATGCCACCACCATTGTCCAGAAGACTCAGTCAACAAT
ATTTGCAGCACAGACGGATATTGTTTCACGATGATAGAAGAGGATGACTC
TGGGTTGCCTGTGGTCACTTCTGGTTGCCTAGGACTAGAAGGCTCAGATT
TTCAGTGTCGGGACACTCCCATTCCTCATCAAAGAAGATCAATTGAATGC
TGCACAGAAAGGAACGAATGTAATAAAGACCTACACCCTACACTGCCTCC
ATTGAAAAACAGAGATTTTGTTGATGGACCTATACACCACAGG

In certain embodiments, the disclosure relates to heteromultimer complexes that comprise at least one ALK6 polypeptide, which includes fragments, functional variants, and modified forms thereof. Preferably, ALK6 polypeptides for use in accordance with inventions of the disclosure (e.g., heteromultimer complexes comprising an ALK6 polypeptide and uses thereof) are soluble (e.g., an extracellular domain of ALK6). In other preferred embodiments, ALK6 polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one ALK6 polypeptide that is at least 70%, 75%, 80%, 85%, 90%, 95%, 97%, 98%, or 99% identical to the amino acid sequence of SEQ ID NO: 34, 35, 91, or 92. In some embodiments, heteromultimer complexes of the disclosure consist or consist essentially of at least one ALK6 polypeptide that is at least 70%, 75%, 80%, 85%, 90%, 95%, 97%, 98%, or 99% identical to the amino acid sequence of SEQ ID NO: 34, 35, 91, or 92.

In certain aspects, the present disclosure relates to protein complexes that comprise an ALK7 polypeptide. As used herein, the term “ALK7” refers to a family of activin receptor-like kinase-7 proteins from any species and variants derived from such ALK7 proteins by mutagenesis or other modification. Reference to ALK7 herein is understood to be a reference to any one of the currently identified forms. Members of the ALK7 family are generally transmembrane proteins, composed of a ligand-binding extracellular domain with a cysteine-rich region, a transmembrane domain, and a cytoplasmic domain with predicted serine/threonine kinase activity.

The term “ALK7 polypeptide” includes polypeptides comprising any naturally occurring polypeptide of an ALK7 family member as well as any variants thereof (including mutants, fragments, fusions, and peptidomimetic forms) that retain a useful activity. Numbering of amino acids for all ALK7-related polypeptides described herein is based on the numbering of the human ALK7 precursor protein sequence below (SEQ ID NO: 38), unless specifically designated otherwise.

Four naturally occurring isoforms of human ALK7 have been described. The sequence of canonical human ALK7 isoform 1 precursor protein (NCBI Ref Seq NP_660302.2) is as follows:

| (SEQ ID NO: 38) | |
|---|---|
| 1 | MTRALCSALR QALLLLAAAA ELSPGLKCVC LLCDSSNFTC QTEGACWASV MLTNGKEQVI |
| 61 | KSCVSLPELN AQVFCHSSNN VTKTECCFTD FCNNITLHLP TASPNAPKLG PMELAIIITV |
| 121 | PVCLLSIAAM LTVWACQGRQ CSYRKKKRPN VEEPLSECNL VNAGKTLKDL IYDVTASGSG |
| 181 | SGLPLLVQRT IARTIVLQEI VGKGRFGEVW HGRWCGEDVA VKIFSSRDER SWFREAEIYQ |
| 241 | TVMLRHENIL GFIAADNKDN GTWTQLWLVS EYHEQGSLYD YLNRNIVTVA GMIKLALSIA |
| 301 | SGLAHLHMEI VGTQGKPAIA HRDIKSKNIL VKKCETCAIA DLGLAVKHDS ILNTIDIPQN |
| 361 | PKVGTKRYMA PEMLDDTMNV NIFESFKRAD IYSVGLVYWE IARRCSVGGI VEEYQLPYYD |
| 421 | MVPSDPSIEE MRKVVCDQKF RPSIPNQWQS CEALRVMGRI MRECWYANGA ARLTALRIKK |
| 481 | TISQLCVKED CKA |

The signal peptide is indicated by a single underline and the extracellular domain is indicated in bold font.

The processed extracellular ALK7 isoform 1 polypeptide sequence is as follows:

(SEQ ID NO: 39)
ELSPGLKCVCLLCDSSNFTCQTEGACWASVMLTNGKEQVIKSCVSLPELN
AQVFCHSSNNVTKTECCFTDFCNNITLHLPTASPNAPKLGPME

A nucleic acid sequence encoding human ALK7 isoform 1 precursor protein is shown below (SEQ ID NO: 40), corresponding to nucleotides 244-1722 of Genbank Reference Sequence NM_145259.2. The signal sequence is underlined and the extracellular domain is indicated in bold font.

(SEQ ID NO: 40)
ATGACCCGGGCGCTCTGCTCAGCGCTCCGCCAGGCTCTCCTGCTGCTCGC
AGCGGCCGCCGAGCTCTCGCCAGGACTGAAGTGTGTATGTCTTTTGTGTG
ATTCTTCAAACTTTACCTGCCAAACAGAAGGAGCATGTTGGGCATCAGTC
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGCATCACCAAATGCCCCAAAACTTGGACCCATGGAGCTGGCCATCAT
TATTACTGTGCCTGTTTGCCTCCTGTCCATAGCTGCGATGCTGACAGTAT
GGGCATGCCAGGGTCGACAGTGCTCCTACAGGAAGAAAAAGAGACCAAAT
GTGGAGGAACCACTCTCTGAGTGCAATCTGGTAAATGCTGGAAAAACTCT
GAAAGATCTGATTTATGATGTGACCGCCTCTGGATCTGGCTCTGGTCTAC
CTCTGTTGGTTCAAAGGACAATTGCAAGGACGATTGTGCTTCAGGAAATA
GTAGGAAAAGGTAGATTTGGTGAGGTGTGGCATGGAAGATGGTGTGGGGA
AGATGTGGCTGTGAAAATATTCTCCTCCAGAGATGAAAGATCTTGGTTTC
GTGAGGCAGAAATTTACCAGACGGTCATGCTGCGACATGAAAACATCCTT
GGTTTCATTGCTGCTGACAACAAAGATAATGGAACTTGGACTCAACTTTG
GCTGGTATCTGAATATCATGAACAGGGCTCCTTATATGACTATTTGAATA
GAAATATAGTGACCGTGGCTGGAATGATCAAGCTGGCGCTCTCAATTGCT
AGTGGTCTGGCACACCTTCATATGGAGATTGTTGGTACACAAGGTAAACC
TGCTATTGCTCATCGAGACATAAAATCAAAGAATATCTTAGTGAAAAAGT
GTGAAACTTGTGCCATAGCGGACTTAGGGTTGGCTGTGAAGCATGATTCA
ATACTGAACACTATCGACATACCTCAGAATCCTAAAGTGGGAACCAAGAG
GTATATGGCTCCTGAAATGCTTGATGATACAATGAATGTGAATATCTTTG
AGTCCTTCAAACGAGCTGACATCTATTCTGTTGGTCTGGTTTACTGGGAA
ATAGCCCGGAGGTGTTCAGTCGGAGGAATTGTTGAGGAGTACCAATTGCC
TTATTATGACATGGTGCCTTCAGATCCCTCGATAGAGGAAATGAGAAAGG
TTGTTTGTGACCAGAAGTTTCGACCAAGTATCCCAAACCAGTGGCAAAGT
TGTGAAGCACTCCGAGTCATGGGGAGAATAATGCGTGAGTGTTGGTATGC
CAACGGAGCGGCCCGCCTAACTGCTCTTCGTATTAAGAAGACTATATCTC
AACTTTGTGTCAAAGAAGACTGCAAAGCC

A nucleic acid sequence encoding the processed extracellular ALK7 polypeptide (isoform 1) is as follows:

(SEQ ID NO: 41)
GAGCTCTCGCCAGGACTGAAGTGTGTATGTCTTTTGTGTGATTCTTCAAA
CTTTACCTGCCAAACAGAAGGAGCATGTTGGGCATCAGTCATGCTAACCA
ATGGAAAAGAGCAGGTGATCAAATCCTGTGTCTCCCTTCCAGAACTGAAT
GCTCAAGTCTTCTGTCATAGTTCCAACAATGTTACCAAAACCGAATGCTG
CTTCACAGATTTTTGCAACAACATAACACTGCACCTTCCAACAGCATCAC
CAAATGCCCCAAAACTTGGACCCATGGAG

The amino acid sequence of an alternative isoform of human ALK7, isoform 2 (NCBI Ref Seq NP_001104501.1), is shown in its processed form as follows (SEQ ID NO: 301), where the extracellular domain is indicated in bold font.

| (SEQ ID NO: 301) | |
|---|---|
| 1 | MLTNGKEQVI KSCVSLPELN AQVFCHSSNN VTKTECCFTD FCNNITLHLP TASPNAPKLG |
| 61 | PMELAIIITV PVCLLSIAAM LTVWACQGRQ CSYRKKKRPN VEEPLSECNL VNAGKTLKDL |
| 121 | IYDVTASGSG SGLPLLVQRT IARTIVLQEI VGKGRFGEVW HGRWCGEDVA VKIFSSRDER |
| 181 | SWFREAEIYQ TVMLRHENIL GFIAADNKDN GTWTQLWLVS EYHEQGSLYD YLNRNIVTVA |
| 241 | GMIKLALSIA SGLAHLHMEI VGTQGKPAIA HRDIKSKNIL VKKCETCAIA DLGLAVKHDS |
| 301 | ILNTIDIPQN PKVGTKRYMA PEMLDDTMNV NIFESFKRAD IYSVGLVYWE IARRCSVGGI |
| 361 | VEEYQLPYYD MVPSDPSIEE MRKVVCDQKF RPSIPNQWQS CEALRVMGRI MRECWYANGA |
| 421 | ARLTALRIKK TISQLCVKED CKA |

The amino acid sequence of the extracellular ALK7 polypeptide (isoform 2) is as follows:

(SEQ ID NO: 302)
MLTNGKEQVIKSCVSLPELNAQVFCHSSNNVTKTECCFTDFCNNITLHLP
TASPNAPKLGPME.

A nucleic acid sequence encoding the processed ALK7 polypeptide (isoform 2) is shown below (SEQ ID NO: 303), corresponding to nucleotides 279-1607 of NCBI Reference Sequence NM_001111031.1. The extracellular domain is indicated in bold font.

(SEQ ID NO: 303)
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGCATCACCAAATGCCCCAAAACTTGGACCCATGGAGCTGGCCATCAT
TATTACTGTGCCTGTTTGCCTCCTGTCCATAGCTGCGATGCTGACAGTAT
GGGCATGCCAGGGTCGACAGTGCTCCTACAGGAAGAAAAAGAGACCAAAT
GTGGAGGAACCACTCTCTGAGTGCAATCTGGTAAATGCTGGAAAAACTCT
GAAAGATCTGATTTATGATGTGACCGCCTCTGGATCTGGCTCTGGTCTAC
CTCTGTTGGTTCAAAGGACAATTGCAAGGACGATTGTGCTTCAGGAAATA
GTAGGAAAAGGTAGATTTGGTGAGGTGTGGCATGGAAGATGGTGTGGGGA
AGATGTGGCTGTGAAAATATTCTCCTCCAGAGATGAAAGATCTTGGTTTC
GTGAGGCAGAAATTTACCAGACGGTCATGCTGCGACATGAAAACATCCTT
GGTTTCATTGCTGCTGACAACAAAGATAATGGAACTTGGACTCAACTTTG
GCTGGTATCTGAATATCATGAACAGGGCTCCTTATATGACTATTTGAATA
GAAATATAGTGACCGTGGCTGGAATGATCAAGCTGGCGCTCTCAATTGCT
AGTGGTCTGGCACACCTTCATATGGAGATTGTTGGTACACAAGGTAAACC
TGCTATTGCTCATCGAGACATAAAATCAAAGAATATCTTAGTGAAAAAGT
GTGAAACTTGTGCCATAGCGGACTTAGGGTTGGCTGTGAAGCATGATTCA
ATACTGAACACTATCGACATACCTCAGAATCCTAAAGTGGGAACCAAGAG
GTATATGGCTCCTGAAATGCTTGATGATACAATGAATGTGAATATCTTTG
AGTCCTTCAAACGAGCTGACATCTATTCTGTTGGTCTGGTTTACTGGGAA
ATAGCCCGGAGGTGTTCAGTCGGAGGAATTGTTGAGGAGTACCAATTGCC
TTATTATGACATGGTGCCTTCAGATCCCTCGATAGAGGAAATGAGAAAGG
TTGTTTGTGACCAGAAGTTTCGACCAAGTATCCCAAACCAGTGGCAAAGT
TGTGAAGCACTCCGAGTCATGGGGAGAATAATGCGTGAGTGTTGGTATGC
CAACGGAGCGGCCCGCCTAACTGCTCTTCGTATTAAGAAGACTATATCTC
AACTTTGTGTCAAAGAAGACTGCAAAGCC

A nucleic acid sequence encoding the extracellular ALK7 polypeptide (isoform 2) is as follows (SEQ ID NO: 304):

(SEQ ID NO: 304)
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGCATCACCAAATGCCCCAAAACTTGGACCCATGGAG

The amino acid sequence of an alternative human ALK7 precursor protein, isoform 3 (NCBI Ref Seq NP_001104502.1), is shown as follows (SEQ ID NO: 305), where the signal peptide is indicated by a single underline.

| (SEQ ID NO: 305) | |
|---|---|
| 1 | MTRALCSALR QALLLLAAAA ELSPGLKCVC LLCDSSNFTC QTEGACWASV MLTNGKEQVI |
| 61 | KSCVSLPELN AQVFCHSSNN VTKTECCFTD FCNNITLHLP TGLPLLVQRT IARTIVLQEI |
| 121 | VGKGRFGEVW HGRWCGEDVA VKIFSSRDER SWFREAEIYQ TVMLRHENIL GFIAADNKDN |
| 181 | GTWTQLWLVS EYHEQGSLYD YLNRNIVTVA GMIKLALSIA SGLAHLHMEI VGTQGKPAIA |
| 241 | HRDIKSKNIL VKKCETCAIA DLGLAVKHDS ILNTIDIPQN PKVGTKRYMA PEMLDDTMNV |
| 301 | NIFESFKRAD IYSVGLVYWE IARRCSVGGI VEEYQLPYYD MVPSDPSIEE MRKVVCDQKF |
| 361 | RPSIPNQWQS CEALRVMGRI MRECWYANGA ARLTALRIKK TISQLCVKED CKA |

The amino acid sequence of the processed ALK7 polypeptide (isoform 3) is as follows (SEQ ID NO: 306). This isoform lacks a transmembrane domain and is therefore proposed to be soluble in its entirety (Roberts et al., 2003, Biol Reprod 68:1719-1726). N-terminal variants of SEQ ID NO: 306 are predicted as described below.

| (SEQ ID NO: 306) | |
|---|---|
| 1 | ELSPGLKCVC LLCDSSNFTC QTEGACWASV MLTNGKEQVI KSCVSLPELN AQVFCHSSNN |
| 61 | VTKTECCFTD FCNNITLHLP TGLPLLVQRT IARTIVLQEI VGKGRFGEVW HGRWCGEDVA |
| 121 | VKIFSSRDER SWFREAEIYQ TVMLRHENIL GFIAADNKDN GTWTQLWLVS EYHEQGSLYD |
| 181 | YLNRNIVTVA GMIKLALSIA SGLAHLHMEI VGTQGKPAIA HRDIKSKNIL VKKCETCAIA |
| 241 | DLGLAVKHDS ILNTIDIPQN PKVGTKRYMA PEMLDDTMNV NIFESFKRAD IYSVGLVYWE |
| 301 | IARRCSVGGI VEEYQLPYYD MVPSDPSIEE MRKVVCDQKF RPSIPNQWQS CEALRVMGRI |
| 361 | MRECWYANGA ARLTALRIKK TISQLCVKED CKA |

A nucleic acid sequence encoding the unprocessed ALK7 polypeptide precursor protein (isoform 3) is shown below (SEQ ID NO: 307), corresponding to nucleotides 244-1482 of NCBI Reference Sequence NM_001111032.1. The signal sequence is indicated by solid underline.

(SEQ ID NO: 307)
ATGACCCGGGCGCTCTGCTCAGCGCTCCGCCAGGCTCTCCTGCTGCTCGC
AGCGGCCGCCGAGCTCTCGCCAGGACTGAAGTGTGTATGTCTTTTGTGTG
ATTCTTCAAACTTTACCTGCCAAACAGAAGGAGCATGTTGGGCATCAGTC
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGGTCTACCTCTGTTGGTTCAAAGGACAATTGCAAGGACGATTGTGCT
TCAGGAAATAGTAGGAAAAGGTAGATTTGGTGAGGTGTGGCATGGAAGAT
GGTGTGGGGAAGATGTGGCTGTGAAAATATTCTCCTCCAGAGATGAAAGA
TCTTGGTTTCGTGAGGCAGAAATTTACCAGACGGTCATGCTGCGACATGA
AAACATCCTTGGTTTCATTGCTGCTGACAACAAAGATAATGGAACTTGGA
CTCAACTTTGGCTGGTATCTGAATATCATGAACAGGGCTCCTTATATGAC
TATTTGAATAGAAATATAGTGACCGTGGCTGGAATGATCAAGCTGGCGCT
CTCAATTGCTAGTGGTCTGGCACACCTTCATATGGAGATTGTTGGTACAC
AAGGTAAACCTGCTATTGCTCATCGAGACATAAAATCAAAGAATATCTTA
GTGAAAAAGTGTGAAACTTGTGCCATAGCGGACTTAGGGTTGGCTGTGAA
GCATGATTCAATACTGAACACTATCGACATACCTCAGAATCCTAAAGTGG
GAACCAAGAGGTATATGGCTCCTGAAATGCTTGATGATACAATGAATGTG
AATATCTTTGAGTCCTTCAAACGAGCTGACATCTATTCTGTTGGTCTGGT
TTACTGGGAAATAGCCCGGAGGTGTTCAGTCGGAGGAATTGTTGAGGAGT
ACCAATTGCCTTATTATGACATGGTGCCTTCAGATCCCTCGATAGAGGAA
ATGAGAAAGGTTGTTTGTGACCAGAAGTTTCGACCAAGTATCCCAAACCA
GTGGCAAAGTTGTGAAGCACTCCGAGTCATGGGGAGAATAATGCGTGAGT
GTTGGTATGCCAACGGAGCGGCCCGCCTAACTGCTCTTCGTATTAAGAAG
ACTATATCTCAACTTTGTGTCAAAGAAGACTGCAAAGCC

A nucleic acid sequence encoding the processed ALK7 polypeptide (isoform 3) is as follows (SEQ ID NO: 308):

(SEQ ID NO: 308)
GAGCTCTCGCCAGGACTGAAGTGTGTATGTCTTTTGTGTGATTCTTCAAA
CTTTACCTGCCAAACAGAAGGAGCATGTTGGGCATCAGTCATGCTAACCA
ATGGAAAAGAGCAGGTGATCAAATCCTGTGTCTCCCTTCCAGAACTGAAT
GCTCAAGTCTTCTGTCATAGTTCCAACAATGTTACCAAAACCGAATGCTG
CTTCACAGATTTTTGCAACAACATAACACTGCACCTTCCAACAGGTCTAC
CTCTGTTGGTTCAAAGGACAATTGCAAGGACGATTGTGCTTCAGGAAATA
GTAGGAAAAGGTAGATTTGGTGAGGTGTGGCATGGAAGATGGTGTGGGGA
AGATGTGGCTGTGAAAATATTCTCCTCCAGAGATGAAAGATCTTGGTTTC
GTGAGGCAGAAATTTACCAGACGGTCATGCTGCGACATGAAAACATCCTT
GGTTTCATTGCTGCTGACAACAAAGATAATGGAACTTGGACTCAACTTTG
GCTGGTATCTGAATATCATGAACAGGGCTCCTTATATGACTATTTGAATA
GAAATATAGTGACCGTGGCTGGAATGATCAAGCTGGCGCTCTCAATTGCT
AGTGGTCTGGCACACCTTCATATGGAGATTGTTGGTACACAAGGTAAACC
TGCTATTGCTCATCGAGACATAAAATCAAAGAATATCTTAGTGAAAAAGT
GTGAAACTTGTGCCATAGCGGACTTAGGGTTGGCTGTGAAGCATGATTCA
ATACTGAACACTATCGACATACCTCAGAATCCTAAAGTGGGAACCAAGAG
GTATATGGCTCCTGAAATGCTTGATGATACAATGAATGTGAATATCTTTG
AGTCCTTCAAACGAGCTGACATCTATTCTGTTGGTCTGGTTTACTGGGAA
ATAGCCCGGAGGTGTTCAGTCGGAGGAATTGTTGAGGAGTACCAATTGCC
TTATTATGACATGGTGCCTTCAGATCCCTCGATAGAGGAAATGAGAAAGG
TTGTTTGTGACCAGAAGTTTCGACCAAGTATCCCAAACCAGTGGCAAAGT
TGTGAAGCACTCCGAGTCATGGGGAGAATAATGCGTGAGTGTTGGTATGC
CAACGGAGCGGCCCGCCTAACTGCTCTTCGTATTAAGAAGACTATATCTC
AACTTTGTGTCAAAGAAGACTGCAAAGCC

The amino acid sequence of an alternative human ALK7 precursor protein, isoform 4 (NCBI Ref Seq NP_001104503.1), is shown as follows (SEQ ID NO: 309), where the signal peptide is indicated by a single underline.

| (SEQ ID NO: 309) | |
|---|---|
| 1 | MTRALCSALR QALLLLAAAA ELSPGLKCVC LLCDSSNFTC QTEGACWASV MLTNGKEQVI |
| 61 | KSCVSLPELN AQVFCHSSNN VTKTECCFTD FCNNITLHLP TDNGTWTQLW LVSEYHEQGS |
| 121 | LYDYLNRNIV TVAGMIKLAL SIASGLAHLH MEIVGTQGKP AIAHRDIKSK NILVKKCETC |
| 181 | AIADLGLAVK HDSILNTIDI PQNPKVGTKR YMAPEMLDDT MNVNIFESFK RADIYSVGLV |
| 241 | YWEIARRCSV GGIVEEYQLP YYDMVPSDPS IEEMRKVVCD QKFRPSIPNQ WQSCEALRVM |
| 301 | GRIMRECWYA NGAARLTALR IKKTISQLCV KEDCKA |

The amino acid sequence of the processed ALK7 polypeptide (isoform 4) is as follows (SEQ ID NO: 310). Like ALK7 isoform 3, isoform 4 lacks a transmembrane domain and is therefore proposed to be soluble in its entirety (Roberts et al., 2003, Biol Reprod 68:1719-1726). N-terminal variants of SEQ ID NO: 310 are predicted as described below.

| (SEQ ID NO: 310) | |
|---|---|
| 1 | ELSPGLKCVC LLCDSSNFTC QTEGACWASV MLTNGKEQVI KSCVSLPELN AQVFCHSSNN |
| 61 | VTKTECCFTD FCNNITLHLP TDNGTWTQLW LVSEYHEQGS LYDYLNRNIV TVAGMIKLAL |
| 121 | SIASGLAHLH MEIVGTQGKP AIAHRDIKSK NILVKKCETC AIADLGLAVK HDSILNTIDI |
| 181 | PQNPKVGTKR YMAPEMLDDT MNVNIFESFK RADIYSVGLV YWEIARRCSV GGIVEEYQLP |
| 240 | YYDMVPSDPS IEEMRKVVCD QKFRPSIPNQ WQSCEALRVM GRIMRECWYA NGAARLTALR |
| 301 | IKKTISQLCV KEDCKA |

A nucleic acid sequence encoding the unprocessed ALK7 polypeptide precursor protein (isoform 4) is shown below (SEQ ID NO: 311), corresponding to nucleotides 244-1244 of NCBI Reference Sequence NM_001111033.1. The signal sequence is indicated by solid underline.

(SEQ ID NO: 311)
ATGACCCGGGCGCTCTGCTCAGCGCTCCGCCAGGCTCTCCTGCTGCTCGC
AGCGGCCGCCGAGCTCTCGCCAGGACTGAAGTGTGTATGTCTTTTGTGTG
ATTCTTCAAACTTTACCTGCCAAACAGAAGGAGCATGTTGGGCATCAGTC
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGATAATGGAACTTGGACTCAACTTTGGCTGGTATCTGAATATCATGA
ACAGGGCTCCTTATATGACTATTTGAATAGAAATATAGTGACCGTGGCTG
GAATGATCAAGCTGGCGCTCTCAATTGCTAGTGGTCTGGCACACCTTCAT
ATGGAGATTGTTGGTACACAAGGTAAACCTGCTATTGCTCATCGAGACAT
AAAATCAAAGAATATCTTAGTGAAAAAGTGTGAAACTTGTGCCATAGCGG
ACTTAGGGTTGGCTGTGAAGCATGATTCAATACTGAACACTATCGACATA
CCTCAGAATCCTAAAGTGGGAACCAAGAGGTATATGGCTCCTGAAATGCT
TGATGATACAATGAATGTGAATATCTTTGAGTCCTTCAAACGAGCTGACA
TCTATTCTGTTGGTCTGGTTTACTGGGAAATAGCCCGGAGGTGTTCAGTC
GGAGGAATTGTTGAGGAGTACCAATTGCCTTATTATGACATGGTGCCTTC
AGATCCCTCGATAGAGGAAATGAGAAAGGTTGTTTGTGACCAGAAGTTTC
GACCAAGTATCCCAAACCAGTGGCAAAGTTGTGAAGCACTCCGAGTCATG
GGGAGAATAATGCGTGAGTGTTGGTATGCCAACGGAGCGGCCCGCCTAAC
TGCTCTTCGTATTAAGAAGACTATATCTCAACTTTGTGTCAAAGAAGACT
GCAAAGCCTAA

A nucleic acid sequence encoding the processed ALK7 polypeptide (isoform 4) is as follows (SEQ ID NO: 312):

(SEQ ID NO: 312)
GAGCTCTCGCCAGGACTGAAGTGTGTATGTCTTTTGTGTGATTCTTCAAA
CTTTACCTGCCAAACAGAAGGAGCATGTTGGGCATCAGTCATGCTAACCA
ATGGAAAAGAGCAGGTGATCAAATCCTGTGTCTCCCTTCCAGAACTGAAT
GCTCAAGTCTTCTGTCATAGTTCCAACAATGTTACCAAAACCGAATGCTG
CTTCACAGATTTTTGCAACAACATAACACTGCACCTTCCAACAGATAATG
GAACTTGGACTCAACTTTGGCTGGTATCTGAATATCATGAACAGGGCTCC
TTATATGACTATTTGAATAGAAATATAGTGACCGTGGCTGGAATGATCAA
GCTGGCGCTCTCAATTGCTAGTGGTCTGGCACACCTTCATATGGAGATTG
TTGGTACACAAGGTAAACCTGCTATTGCTCATCGAGACATAAAATCAAAG
AATATCTTAGTGAAAAAGTGTGAAACTTGTGCCATAGCGGACTTAGGGTT
GGCTGTGAAGCATGATTCAATACTGAACACTATCGACATACCTCAGAATC
CTAAAGTGGGAACCAAGAGGTATATGGCTCCTGAAATGCTTGATGATACA
ATGAATGTGAATATCTTTGAGTCCTTCAAACGAGCTGACATCTATTCTGT
TGGTCTGGTTTACTGGGAAATAGCCCGGAGGTGTTCAGTCGGAGGAATTG
TTGAGGAGTACCAATTGCCTTATTATGACATGGTGCCTTCAGATCCCTCG
ATAGAGGAAATGAGAAAGGTTGTTTGTGACCAGAAGTTTCGACCAAGTAT
CCCAAACCAGTGGCAAAGTTGTGAAGCACTCCGAGTCATGGGGAGAATAA
TGCGTGAGTGTTGGTATGCCAACGGAGCGGCCCGCCTAACTGCTCTTCGT
ATTAAGAAGACTATATCTCAACTTTGTGTCAAAGAAGACTGCAAAGC
CTAA

Based on the signal sequence of full-length ALK7 (isoform 1) in the rat (see NCBI Reference Sequence NP_620790.1) and on the high degree of sequence identity between human and rat ALK7, it is predicted that a processed form of human ALK7 isoform 1 is as follows (SEQ ID NO: 313).

| (SEQ ID NO: 313) | |
|---|---|
| 1 | LKCVCLLCDS SNFTCQTEGA CWASVMLTNG KEQVIKSCVS LPELNAQVFC HSSNNVTKTE |
| 61 | CCFTDFCNNI TLHLPTASPN APKLGPME |

Active variants of processed ALK7 isoform 1 are predicted in which SEQ ID NO: 39 is truncated by 1, 2, 3, 4, 5, 6, or 7 amino acids at the N-terminus and SEQ ID NO: 313 is truncated by 1 or 2 amino acids at the N-terminus. Consistent with SEQ ID NO: 313, it is further expected that leucine is the N-terminal amino acid in the processed forms of human ALK7 isoform 3 (SEQ ID NO: 306) and human ALK7 isoform 4 (SEQ ID NO: 310).

In certain embodiments, the disclosure relates to heteromultimer complexes that comprise at least one ALK7 polypeptide, which includes fragments, functional variants, and modified forms thereof. Preferably, ALK7 polypeptides for use in accordance with inventions of the disclosure (e.g., heteromultimer complexes comprising an ALK7 polypeptide and uses thereof) are soluble (e.g., an extracellular domain of ALK7). In other preferred embodiments, ALK7 polypeptides for use in accordance with the inventions of the disclosure bind to and/or inhibit (antagonize) activity (e.g., induction of Smad 2/3 and/or Smad 1/5/8 signaling) of one or more TGF-beta superfamily ligands. In some embodiments, heteromultimer complexes of the disclosure comprise at least one ALK7 polypeptide that is at least 70%, 75%, 80%, 85%, 90%, 95%, 97%, 98%, or 99% identical to the amino acid sequence of SEQ ID NO: 38, 39, 112, 114, 301, 302, 305, 306, 309, 310, 313, 405, or 406. In some embodiments, heteromultimer complexes of the disclosure consist or consist essentially of at least one ALK7 polypeptide that is at least 70%, 75%, 80%, 85%, 90%, 95%, 97%, 98%, or 99% identical to the amino acid sequence of SEQ ID NO: 38, 39, 112, 114, 301, 302, 305, 306, 309, 310, 313, 405, or 406.

In certain aspects, the disclosure relates to heteromultimers that comprise at least one ALK1 polypeptide, which includes fragments, functional variants, and modified forms thereof, and at least one ActRIIB polypeptide, which includes fragments, functional variants, and modified forms thereof. In some embodiments, ALK1:ActRIIB heteromultimer complexes of the disclosure comprise at least one ALK1 polypeptide that comprises, consists of, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ActRIIB heteromultimers of the disclosure comprises at least one ActRIIB polypeptide that comprises, consists, or consists essentially a sequence that is at least 70%, 75%, 80%, 85%, 90%, 91%, 92%, 93%, 94%, 95%, 96% 97%, 98%, 99%, or 100% identical to amino acids 29-109 of SEQ ID NO: 1. In some embodiments, ALK1:ActRIIB heteromultimer complexes of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 1, 2, 3, 4, 5, 6, 100, 102, 153, 154, 401, 402, 453, and 454. In certain preferred embodiments, ActRIIB polypeptides of the disclosure do not comprise an acidic amino acid (e.g., a naturally occurring D or E amino acid or artificially acidic amino acid). Preferably, ALK1:ActRIIB heteromultimer complexes of the present disclosure are soluble (e.g., comprise an extracellular domain of ALK1 and/or ActRIIB). In other embodiments, ALK1:ActRIIB heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ActRIIB heteromultimer complexes of the disclosure have different ligand binding specificities/profiles compared to their corresponding homomultimer complexes (i.e., ALK1 and ActRIIB homomultimers). ALK1:ActRIIB heteromultimer complexes of the disclosure include, e.g., heterodimers, heterotrimers, heterotetramers and further oligomeric structures. In certain preferred embodiments, ALK1:ActRIIB heteromultimer complexes of the disclosure are heterodimers.

In certain aspects, the disclosure relates to heteromultimers that comprise at least one ALK2 polypeptide, which includes fragments, functional variants, and modified forms thereof, and at least one ActRIIB polypeptide, which includes fragments, functional variants, and modified forms thereof. In some embodiments, ALK2:ActRIIB heteromultimer complexes of the disclosure comprise at least one ALK2 polypeptide that comprises, consists of, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 18, 19, 136, 138, 173, 174, 421, 422, 465, and 466 or amino acids 35-99 of SEQ ID NO: 18. In some embodiments, ALK2:ActRIIB heteromultimers of the disclosure comprises at least one ActRIIB polypeptide that comprises, consists, or consists essentially a sequence that is at least 70%, 75%, 80%, 85%, 90%, 91%, 92%, 93%, 94%, 95%, 96% 97%, 98%, 99%, or 100% identical to amino acids 29-109 of SEQ ID NO: 1. In some embodiments, ALK2:ActRIIB heteromultimer complexes of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 1, 2, 3, 4, 5, 6, 100, 102, 153, 154, 401, 402, 453, and 454. In certain preferred embodiments, ActRIIB polypeptides of the disclosure do not comprise an acidic amino acid (e.g., a naturally occurring D or E amino acid or artificially acidic amino acid). Preferably, ALK2:ActRIIB heteromultimer complexes of the present disclosure are soluble (e.g., comprise an extracellular domain of ALK2 and/or ActRIIB). In other embodiments, ALK2:ActRIIB heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:ActRIIB heteromultimer complexes of the disclosure have different ligand binding specificities/profiles compared to their corresponding homomultimer complexes (i.e., ALK2 and ActRIIB homomultimers). ALK2:ActRIIB heteromultimer complexes of the disclosure include, e.g., heterodimers, heterotrimers, heterotetramers and further oligomeric structures. In certain preferred embodiments, ALK2:ActRIIB heteromultimer complexes of the disclosure are heterodimers.

In certain aspects, the disclosure relates to heteromultimers that comprise at least one ALK3 polypeptide, which includes fragments, functional variants, and modified forms thereof, and at least one ActRIIB polypeptide, which includes fragments, functional variants, and modified forms thereof. In some embodiments, ALK3:ActRIIB heteromultimer complexes of the disclosure comprise at least one ALK3 polypeptide that comprises, consists of, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 22, 23, 115, 117, 175, 176, 407, 408, 467, and 468 or amino acids 61-130 of SEQ ID NO: 22. In some embodiments, ALK3:ActRIIB heteromultimers of the disclosure comprises at least one ActRIIB polypeptide that comprises, consists, or consists essentially a sequence that is at least 70%, 75%, 80%, 85%, 90%, 91%, 92%, 93%, 94%, 95%, 96% 97%, 98%, 99%, or 100% identical to amino acids 29-109 of SEQ ID NO: 1. In some embodiments, ALK3:ActRIIB heteromultimer complexes of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 1, 2, 3, 4, 5, 6, 100, 102, 153, 154, 401, 402, 453, and 454. In certain preferred embodiments, ActRIIB polypeptides of the disclosure do not comprise an acidic amino acid (e.g., a naturally occurring D or E amino acid or artificially acidic amino acid). Preferably, ALK3:ActRIIB heteromultimer complexes of the present disclosure are soluble (e.g., comprise an extracellular domain of ALK3 and/or ActRIIB). In other embodiments, ALK3:ActRIIB heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK3:ActRIIB heteromultimer complexes of the disclosure have different ligand binding specificities/profiles compared to their corresponding homomultimer complexes (i.e., ALK3 and ActRIIB homomultimers). ALK3:ActRIIB heteromultimer complexes of the disclosure include, e.g., heterodimers, heterotrimers, heterotetramers and further oligomeric structures. In certain preferred embodiments, ALK3:ActRIIB heteromultimer complexes of the disclosure are heterodimers.

In certain aspects, the disclosure relates to heteromultimers that comprise at least one ALK4 polypeptide, which includes fragments, functional variants, and modified forms thereof, and at least one ActRIIB polypeptide, which includes fragments, functional variants, and modified forms thereof. In some embodiments, ALK4:ActRIIB heteromultimer complexes of the disclosure comprise at least one ALK4 polypeptide that comprises, consists of, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 26, 27, 83, 84, 104, 106, 177, 178, 403, 404, 469, and 470 or amino acids 34-101 of SEQ ID NO: 26 or 83. In some embodiments, ALK4:ActRIIB heteromultimers of the disclosure comprises at least one ActRIIB polypeptide that comprises, consists, or consists essentially a sequence that is at least 70%, 75%, 80%, 85%, 90%, 91%, 92%, 93%, 94%, 95%, 96% 97%, 98%, 99%, or 100% identical to amino acids 29-109 of SEQ ID NO: 1. In some embodiments, ALK4:ActRIIB heteromultimer complexes of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 1, 2, 3, 4, 5, 6, 100, 102, 153, 154, 401, 402, 453, and 454. In certain preferred embodiments, ActRIIB polypeptides of the disclosure do not comprise an acidic amino acid (e.g., a naturally occurring D or E amino acid or artificially acidic amino acid). Preferably, ALK4:ActRIIB heteromultimer complexes of the present disclosure are soluble (e.g., comprise an extracellular domain of ALK4 and/or ActRIIB). In other embodiments, ALK4:ActRIIB heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK4:ActRIIB heteromultimer complexes of the disclosure have different ligand binding specificities/profiles compared to their corresponding homomultimer complexes (i.e., ALK4 and ActRIIB homomultimers). ALK4:ActRIIB heteromultimer complexes of the disclosure include, e.g., heterodimers, heterotrimers, heterotetramers and further oligomeric structures. In certain preferred embodiments, ALK4:ActRIIB heteromultimer complexes of the disclosure are heterodimers.

In certain aspects, the disclosure relates to heteromultimers that comprise at least one ALK5 polypeptide, which includes fragments, functional variants, and modified forms thereof, and at least one ActRIIB polypeptide, which includes fragments, functional variants, and modified forms thereof. In some embodiments, ALK5:ActRIIB heteromultimer complexes of the disclosure comprise at least one ALK5 polypeptide that comprises, consists of, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 30, 31, 87, 88, 139, 141, 179, 180, 423, 424, 471, and 472 or amino acids 36-106 of SEQ ID NO: 30 or 87. In some embodiments, ALK5:ActRIIB heteromultimers of the disclosure comprises at least one ActRIIB polypeptide that comprises, consists, or consists essentially a sequence that is at least 70%, 75%, 80%, 85%, 90%, 91%, 92%, 93%, 94%, 95%, 96% 97%, 98%, 99%, or 100% identical to amino acids 29-109 of SEQ ID NO: 1. In some embodiments, ALK5:ActRIIB heteromultimer complexes of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 1, 2, 3, 4, 5, 6, 100, 102, 153, 154, 401, 402, 453, and 454. In certain preferred embodiments, ActRIIB polypeptides of the disclosure do not comprise an acidic amino acid (e.g., a naturally occurring D or E amino acid or artificially acidic amino acid). Preferably, ALK5:ActRIIB heteromultimer complexes of the present disclosure are soluble (e.g., comprise an extracellular domain of ALK5 and/or ActRIIB). In other embodiments, ALK5:ActRIIB heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK5:ActRIIB heteromultimer complexes of the disclosure have different ligand binding specificities/profiles compared to their corresponding homomultimer complexes (i.e., ALK5 and ActRIIB homomultimers). ALK5:ActRIIB heteromultimer complexes of the disclosure include, e.g., heterodimers, heterotrimers, heterotetramers and further oligomeric structures. In certain preferred embodiments, ALK5:ActRIIB heteromultimer complexes of the disclosure are heterodimers.

In certain aspects, the disclosure relates to heteromultimers that comprise at least one ALK6 polypeptide, which includes fragments, functional variants, and modified forms thereof, and at least one ActRIIB polypeptide, which includes fragments, functional variants, and modified forms thereof. In some embodiments, ALK6:ActRIIB heteromultimer complexes of the disclosure comprise at least one ALK6 polypeptide that comprises, consists of, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 34, 35, 91, 92, 142, 144, 181, 182, 425, 426, 473, and 474 or amino acids 32-102 of SEQ ID NO: 34 or amino acids 62-132 of SEQ ID NO: 91. In some embodiments, ALK6:ActRIIB heteromultimers of the disclosure comprises at least one ActRIIB polypeptide that comprises, consists, or consists essentially a sequence that is at least 70%, 75%, 80%, 85%, 90%, 91%, 92%, 93%, 94%, 95%, 96% 97%, 98%, 99%, or 100% identical to amino acids 29-109 of SEQ ID NO: 1. In some embodiments, ALK6:ActRIIB heteromultimer complexes of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 1, 2, 3, 4, 5, 6, 100, 102, 153, 154, 401, 402, 453, and 454. In certain preferred embodiments, ActRIIB polypeptides of the disclosure do not comprise an acidic amino acid (e.g., a naturally occurring D or E amino acid or artificially acidic amino acid). Preferably, ALK6:ActRIIB heteromultimer complexes of the present disclosure are soluble (e.g., comprise an extracellular domain of ALK6 and/or ActRIIB). In other embodiments, ALK6:ActRIIB heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK6:ActRIIB heteromultimer complexes of the disclosure have different ligand binding specificities/profiles compared to their corresponding homomultimer complexes (i.e., ALK6 and ActRIIB homomultimers). ALK6:ActRIIB heteromultimer complexes of the disclosure include, e.g., heterodimers, heterotrimers, heterotetramers and further oligomeric structures. In certain preferred embodiments, ALK6:ActRIIB heteromultimer complexes of the disclosure are heterodimers.

In certain aspects, the disclosure relates to heteromultimers that comprise at least one ALK7 polypeptide, which includes fragments, functional variants, and modified forms thereof, and at least one ActRIIB polypeptide, which includes fragments, functional variants, and modified forms thereof. In some embodiments, ALK7:ActRIIB heteromultimer complexes of the disclosure comprise at least one ALK7 polypeptide that comprises, consists of, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 38, 39, 301, 302, 305, 306, 309, 310, 313, 112, 114, 183, 184, 405, 406, 475, and 476 or amino acids 28-92 of SEQ ID Nos: 38, 305, and 309. In some embodiments, ALK7:ActRIIB heteromultimers of the disclosure comprises at least one ActRIIB polypeptide that comprises, consists, or consists essentially a sequence that is at least 70%, 75%, 80%, 85%, 90%, 91%, 92%, 93%, 94%, 95%, 96% 97%, 98%, 99%, or 100% identical to amino acids 29-109 of SEQ ID NO: 1. In some embodiments, ALK7:ActRIIB heteromultimer complexes of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 1, 2, 3, 4, 5, 6, 100, 102, 153, 154, 401, 402, 453, and 454. In certain preferred embodiments, ActRIIB polypeptides of the disclosure do not comprise an acidic amino acid (e.g., a naturally occurring D or E amino acid or artificially acidic amino acid). Preferably, ALK7:ActRIIB heteromultimer complexes of the present disclosure are soluble (e.g., comprise an extracellular domain of ALK7 and/or ActRIIB). In other embodiments, ALK7:ActRIIB heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK7:ActRIIB heteromultimer complexes of the disclosure have different ligand binding specificities/profiles compared to their corresponding homomultimer complexes (i.e., ALK7 and ActRIIB homomultimers). ALK7:ActRIIB heteromultimer complexes of the disclosure include, e.g., heterodimers, heterotrimers, heterotetramers and further oligomeric structures. In certain preferred embodiments, ALK7:ActRIIB heteromultimer complexes of the disclosure are heterodimers.

In certain aspects, the disclosure relates to heteromultimers that comprise at least one ALK1 polypeptide, which includes fragments, functional variants, and modified forms thereof, and at least one ActRIIA polypeptide, which includes fragments, functional variants, and modified forms thereof. In some embodiments, ALK1:ActRIIA heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ActRIIA heteromultimers of the disclosure comprises at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 9, 10, 11, 118, 120, 151, 152, 409, 410, 451, and 452 or amino acids 30-110 of SEQ ID NO: 9. Preferably, ALK1:ActRIIA heteromultimers of the present disclosure are soluble (e.g., comprise an extracellular domain of ALK1 and/or ActRIIA). In other preferred embodiments, ALK1:ActRIIA heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ActRIIA heteromultimers of the disclosure have different ligand binding specificities/profiles compared to their corresponding homomultimer complexes (i.e., ALK1 and ActRIIA homomultimers). ALK1:ActRIIA heteromultimer complexes of the disclosure include, e.g., heterodimers, heterotrimers, heterotetramers and further oligomeric structures. In certain preferred embodiments, ALK1:ActRIIA heteromultimer complexes of the disclosure are heterodimers.

In certain aspects, the disclosure relates to heteromultimers that comprise at least one ALK2 polypeptide, which includes fragments, functional variants, and modified forms thereof, and at least one ActRIIA polypeptide, which includes fragments, functional variants, and modified forms thereof. In some embodiments, ALK2:ActRIIA heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 18, 19, 136, 138, 173, 174, 421, 422, 465, and 466 or amino acids 35-99 of SEQ ID NO: 18. In some embodiments, ALK2:ActRIIA heteromultimers of the disclosure comprises at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 9, 10, 11, 118, 120, 151, 152, 409, 410, 451, and 452 or amino acids 30-110 of SEQ ID NO: 9. Preferably, ALK2:ActRIIA heteromultimers of the present disclosure are soluble (e.g., comprise an extracellular domain of ALK2 and/or ActRIIA). In other preferred embodiments, ALK2:ActRIIA heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:ActRIIA heteromultimers of the disclosure have different ligand binding specificities/profiles compared to their corresponding homomultimer complexes (i.e., ALK2 and ActRIIA homomultimers). ALK2:ActRIIA heteromultimer complexes of the disclosure include, e.g., heterodimers, heterotrimers, heterotetramers and further oligomeric structures. In certain preferred embodiments, ALK2:ActRIIA heteromultimer complexes of the disclosure are heterodimers.

In certain aspects, the disclosure relates to heteromultimers that comprise at least one ALK3 polypeptide, which includes fragments, functional variants, and modified forms thereof, and at least one ActRIIA polypeptide, which includes fragments, functional variants, and modified forms thereof. In some embodiments, ALK3:ActRIIA heteromultimers of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 22, 23, 115, 117, 175, 176, 407, 408, 467, and 468 or amino acids 61-130 of SEQ ID NO: 22. In some embodiments, ALK3:ActRIIA heteromultimers of the disclosure comprises at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 9, 10, 11, 118, 120, 151, 152, 409, 410, 451, and 452 or amino acids 30-110 of SEQ ID NO: 9. Preferably, ALK3:ActRIIA heteromultimers of the present disclosure are soluble (e.g., comprise an extracellular domain of ALK3 and/or ActRIIA). In other preferred embodiments, ALK3:ActRIIA heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK3:ActRIIA heteromultimers of the disclosure have different ligand binding specificities/profiles compared to their corresponding homomultimer complexes (i.e., ALK3 and ActRIIA homomultimers). ALK3:ActRIIA heteromultimer complexes of the disclosure include, e.g., heterodimers, heterotrimers, heterotetramers and further oligomeric structures. In certain preferred embodiments, ALK3:ActRIIA heteromultimer complexes of the disclosure are heterodimers.

In certain aspects, the disclosure relates to heteromultimers that comprise at least one ALK4 polypeptide, which includes fragments, functional variants, and modified forms thereof, and at least one ActRIIA polypeptide, which includes fragments, functional variants, and modified forms thereof. In some embodiments, ALK4:ActRIIA heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 26, 27, 83, 84, 104, 106, 177, 178, 403, 404, 469, and 470 or amino acids 34-101 of SEQ ID NO: 26 or 83. In some embodiments, ALK4:ActRIIA heteromultimers of the disclosure comprises at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 9, 10, 11, 118, 120, 151, 152, 409, 410, 451, and 452 or amino acids 30-110 of SEQ ID NO: 9. Preferably, ALK4:ActRIIA heteromultimers of the present disclosure are soluble (e.g., comprise an extracellular domain of ALK4 and/or ActRIIA). In other preferred embodiments, ALK4:ActRIIA heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK4:ActRIIA heteromultimers of the disclosure have different ligand binding specificities/profiles compared to their corresponding homomultimer complexes (i.e., ALK4 and ActRIIA homomultimers). ALK4:ActRIIA heteromultimer complexes of the disclosure include, e.g., heterodimers, heterotrimers, heterotetramers and further oligomeric structures. In certain preferred embodiments, ALK4:ActRIIA heteromultimer complexes of the disclosure are heterodimers.

In certain aspects, the disclosure relates to heteromultimers that comprise at least one ALK5 polypeptide, which includes fragments, functional variants, and modified forms thereof, and at least one ActRIIA polypeptide, which includes fragments, functional variants, and modified forms thereof. In some embodiments, ALK5:ActRIIA heteromultimers of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 30, 31, 87, 88, 139, 141, 179, 180, 423, 424, 471, and 472 or amino acids 36-106 of SEQ ID NO: 30 or 87. In some embodiments, ALK5:ActRIIA heteromultimers of the disclosure comprises at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 9, 10, 11, 118, 120, 151, 152, 409, 410, 451, and 452 or amino acids 30-110 of SEQ ID NO: 9. Preferably, ALK5:ActRIIA heteromultimers of the present disclosure are soluble (e.g., comprise an extracellular domain of ALK5 and/or ActRIIA). In other preferred embodiments, ALK5:ActRIIA heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK5:ActRIIA heteromultimers of the disclosure have different ligand binding specificities/profiles compared to their corresponding homomultimer complexes (i.e., ALK5 and ActRIIA homomultimers). ALK5:ActRIIA heteromultimer complexes of the disclosure include, e.g., heterodimers, heterotrimers, heterotetramers and further oligomeric structures. In certain preferred embodiments, ALK5:ActRIIA heteromultimer complexes of the disclosure are heterodimers.

In certain aspects, the disclosure relates to heteromultimers that comprise at least one ALK6 polypeptide, which includes fragments, functional variants, and modified forms thereof, and at least one ActRIIA polypeptide, which includes fragments, functional variants, and modified forms thereof. In some embodiments, ALK6:ActRIIA heteromultimers of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 34, 35, 91, 92, 142, 144, 181, 182, 425, 426, 473, and 474 or amino acids 32-102 of SEQ ID NO: 34 or amino acids 62-132 of SEQ ID NO: 91. In some embodiments, ALK6:ActRIIA heteromultimers of the disclosure comprises at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 9, 10, 11, 118, 120, 151, 152, 409, 410, 451, and 452 or amino acids 30-110 of SEQ ID NO: 9. Preferably, ALK6:ActRIIA heteromultimers of the present disclosure are soluble (e.g., comprise an extracellular domain of ALK6 and/or ActRIIA). In other preferred embodiments, ALK6:ActRIIA heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK6:ActRIIA heteromultimers of the disclosure have different ligand binding specificities/profiles compared to their corresponding homomultimer complexes (i.e., ALK6 and ActRIIA homomultimers). ALK6:ActRIIA heteromultimer complexes of the disclosure include, e.g., heterodimers, heterotrimers, heterotetramers and further oligomeric structures. In certain preferred embodiments, ALK6:ActRIIA heteromultimer complexes of the disclosure are heterodimers.

In certain aspects, the disclosure relates to heteromultimers that comprise at least one ALK7 polypeptide, which includes fragments, functional variants, and modified forms thereof, and at least one ActRIIA polypeptide, which includes fragments, functional variants, and modified forms thereof. In some embodiments, ALK7:ActRIIA heteromultimers of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 38, 39, 301, 302, 305, 306, 309, 310, 313, 112, 114, 183, 184, 405, 406, 475, and 476 or amino acids 28-93 of SEQ ID Nos: 38, 305, or 309. In some embodiments, ALK7:ActRIIA heteromultimers of the disclosure comprises at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NOs: 9, 10, 11, 118, 120, 151, 152, 409, 410, 451, and 452 or amino acids 30-110 of SEQ ID NO: 9. Preferably, ALK7:ActRIIA heteromultimers of the present disclosure are soluble (e.g., comprise an extracellular domain of ALK7 and/or ActRIIA). In other preferred embodiments, ALK7:ActRIIA heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK7:ActRIIA heteromultimers of the disclosure have different ligand binding specificities/profiles compared to their corresponding homomultimer complexes (i.e., ALK7 and ActRIIA homomultimers). ALK7:ActRIIA heteromultimer complexes of the disclosure include, e.g., heterodimers, heterotrimers, heterotetramers and further oligomeric structures. In certain preferred embodiments, ALK7:ActRIIA heteromultimer complexes of the disclosure are heterodimers.

In certain aspects, the disclosure relates to heteromultimers that comprise at least one ALK1 polypeptide, which includes fragments, functional variants, and modified forms thereof, and at least one TGFBRII polypeptide, which includes fragments, functional variants, and modified forms thereof. In some embodiments, ALK1:TGFβRII heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:TGFBRII heteromultimer complexes of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, ALK1:TGFβRII heteromultimer complexes of the present disclosure are soluble (e.g., comprise an extracellular domain of ALK1 and/or TGFβRII). In other preferred embodiments, ALK1:TGFβRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:TGFBRII heteromultimer complexes of the disclosure have different ligand binding specificities/profiles compared to their corresponding homomultimer complexes (i.e., ALK1 and TGFBRII homomultimers). ALK1:TGFBRII heteromultimer complexes of the disclosure include, e.g., heterodimers, heterotrimers, heterotetramers and further oligomeric structures. In certain preferred embodiments, ALK1:TGFBRII heteromultimer complexes of the disclosure are heterodimers.

In certain aspects, the disclosure relates to heteromultimers that comprise at least one ALK2 polypeptide, which includes fragments, functional variants, and modified forms thereof, and at least one TGFBRII polypeptide, which includes fragments, functional variants, and modified forms thereof. In some embodiments, ALK2:TGFBRII heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 18, 19, 136, 138, 173, 174, 421, 422, 465, and 466 or amino acids 35-99 of SEQ ID NO: 18. In some embodiments, ALK2:TGFBRII heteromultimer complexes of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, ALK2:TGFBRII heteromultimer complexes of the present disclosure are soluble (e.g., comprise an extracellular domain of ALK2 and/or TGFBRII). In other preferred embodiments, ALK2:TGFβRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:TGFBRII heteromultimer complexes of the disclosure have different ligand binding specificities/profiles compared to their corresponding homomultimer complexes (i.e., ALK2 and TGFBRII homomultimers). ALK2:TGFBRII heteromultimer complexes of the disclosure include, e.g., heterodimers, heterotrimers, heterotetramers and further oligomeric structures. In certain preferred embodiments, ALK2:TGFBRII heteromultimer complexes of the disclosure are heterodimers.

In certain aspects, the disclosure relates to heteromultimers that comprise at least one ALK3 polypeptide, which includes fragments, functional variants, and modified forms thereof, and at least one TGFBRII polypeptide, which includes fragments, functional variants, and modified forms thereof. In some embodiments, ALK3:TGFBRII heteromultimers of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 22, 23, 115, 117, 175, 176, 407, 408, 467, and 468 or amino acids 61-130 of SEQ ID NO: 22. In some embodiments, ALK3:TGFBRII heteromultimer complexes of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, ALK3:TGFBRII heteromultimer complexes of the present disclosure are soluble (e.g., comprise an extracellular domain of ALK3 and/or TGFBRII). In other preferred embodiments, ALK3:TGFBRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK3:TGFBRII heteromultimer complexes of the disclosure have different ligand binding specificities/profiles compared to their corresponding homomultimer complexes (i.e., ALK3 and TGFBRII homomultimers). ALK3:TGFBRII heteromultimer complexes of the disclosure include, e.g., heterodimers, heterotrimers, heterotetramers and further oligomeric structures. In certain preferred embodiments, ALK3:TGFBRII heteromultimer complexes of the disclosure are heterodimers.

In certain aspects, the disclosure relates to heteromultimers that comprise at least one ALK4 polypeptide, which includes fragments, functional variants, and modified forms thereof, and at least one TGFBRII polypeptide, which includes fragments, functional variants, and modified forms thereof. In some embodiments, ALK4:TGFBRII heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 26, 27, 83, 84, 104, 106, 177, 178, 403, 404, 469, and 470 or amino acids 34-101 of SEQ ID NO: 26 or 83. In some embodiments, ALK4:TGFBRII heteromultimer complexes of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, ALK4:TGFBRII heteromultimer complexes of the present disclosure are soluble (e.g., comprise an extracellular domain of ALK4 and/or TGFβRII). In other preferred embodiments, ALK4:TGFBRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK4:TGFBRII heteromultimer complexes of the disclosure have different ligand binding specificities/profiles compared to their corresponding homomultimer complexes (i.e., ALK4 and TGFBRII homomultimers). ALK4:TGFBRII heteromultimer complexes of the disclosure include, e.g., heterodimers, heterotrimers, heterotetramers and further oligomeric structures. In certain preferred embodiments, ALK4:TGFBRII heteromultimer complexes of the disclosure are heterodimers.

In certain aspects, the disclosure relates to heteromultimers that comprise at least one ALK5 polypeptide, which includes fragments, functional variants, and modified forms thereof, and at least one TGFBRII polypeptide, which includes fragments, functional variants, and modified forms thereof. In some embodiments, ALK5:TGFBRII heteromultimers of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 30, 31, 87, 88, 139, 141, 179, 180, 423, 424, 471, and 472 or amino acids 36-106 of SEQ ID NO: 30 or 87. In some embodiments, ALK5:TGFBRII heteromultimer complexes of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, ALK5:TGFBRII heteromultimer complexes of the present disclosure are soluble (e.g., comprise an extracellular domain of ALK5 and/or TGFβRII). In other preferred embodiments, ALK5:TGFBRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK5:TGFBRII heteromultimer complexes of the disclosure have different ligand binding specificities/profiles compared to their corresponding homomultimer complexes (i.e., ALK5 and TGFBRII homomultimers). ALK5:TGFBRII heteromultimer complexes of the disclosure include, e.g., heterodimers, heterotrimers, heterotetramers and further oligomeric structures. In certain preferred embodiments, ALK5:TGFBRII heteromultimer complexes of the disclosure are heterodimers.

In certain aspects, the disclosure relates to heteromultimers that comprise at least one ALK6 polypeptide, which includes fragments, functional variants, and modified forms thereof, and at least one TGFBRII polypeptide, which includes fragments, functional variants, and modified forms thereof. In some embodiments, ALK6:TGFBRII heteromultimers of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 34, 35, 91, 92, 142, 144, 181, 182, 425, 426, 473, and 474 or amino acids 32-102 of SEQ ID NO: 34 or amino acids 62-132 of SEQ ID NO: 91. In some embodiments, ALK6:TGFBRII heteromultimer complexes of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, ALK6:TGFBRII heteromultimer complexes of the present disclosure are soluble (e.g., comprise an extracellular domain of ALK6 and/or TGFBRII). In other preferred embodiments, ALK6:TGFBRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK6:TGFBRII heteromultimer complexes of the disclosure have different ligand binding specificities/profiles compared to their corresponding homomultimer complexes (i.e., ALK6 and TGFBRII homomultimers). ALK6:TGFBRII heteromultimer complexes of the disclosure include, e.g., heterodimers, heterotrimers, heterotetramers and further oligomeric structures. In certain preferred embodiments, ALK6:TGFBRII heteromultimer complexes of the disclosure are heterodimers.

In certain aspects, the disclosure relates to heteromultimers that comprise at least one ALK7 polypeptide, which includes fragments, functional variants, and modified forms thereof, and at least one TGFBRII polypeptide, which includes fragments, functional variants, and modified forms thereof. In some embodiments, ALK7:TGFBRII heteromultimers of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 38, 39, 301, 302, 305, 306, 309, 310, 313, 112, 114, 183, 184, 405, 406, 475, and 476 or amino acids 28-93 of SEQ ID Nos: 38, 305, or 309. In some embodiments, ALK7:TGFBRII heteromultimer complexes of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, ALK7:TGFBRII heteromultimer complexes of the present disclosure are soluble (e.g., comprise an extracellular domain of ALK7 and/or TGFβRII). In other preferred embodiments, ALK7:TGFBRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK7:TGFBRII heteromultimer complexes of the disclosure have different ligand binding specificities/profiles compared to their corresponding homomultimer complexes (i.e., ALK7 and TGFBRII homomultimers). ALK7:TGFBRII heteromultimer complexes of the disclosure include, e.g., heterodimers, heterotrimers, heterotetramers and further oligomeric structures. In certain preferred embodiments, ALK7:TGFBRII heteromultimer complexes of the disclosure are heterodimers.

In certain aspects, the disclosure relates to heteromultimers that comprise at least one ALK1 polypeptide, which includes fragments, functional variants, and modified forms thereof, and at least one MISRII polypeptide, which includes fragments, functional variants, and modified forms thereof. In some embodiments, ALK1:MISRII heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:MISRII heteromultimer complexes of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 50, 51, 75, 76, 79, 80, 133, 135, 161, 162, 419, 420, 457, and 458 or amino acids 24-116 of SEQ ID Nos: 50, 75, or 79. Preferably, ALK1:MISRII heteromultimer complexes of the present disclosure are soluble (e.g., comprise an extracellular domain of ALK1 and/or MISRII). In other preferred embodiments, ALK1:MISRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:MISRII heteromultimer complexes of the disclosure have different ligand binding specificities/profiles compared to their corresponding homomultimer complexes (i.e., ALK1 and MISRII homomultimers). ALK1:MISRII heteromultimers of the disclosure include, e.g., heterodimers, heterotrimers, heterotetramers and further oligomeric structures. In certain preferred embodiments, ALK1:MISRII heteromultimers of the disclosure are heterodimers.

In certain aspects, the disclosure relates to heteromultimers that comprise at least one ALK2 polypeptide, which includes fragments, functional variants, and modified forms thereof, and at least one MISRII polypeptide, which includes fragments, functional variants, and modified forms thereof. In some embodiments, ALK2:MISRII heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 18, 19, 136, 138, 173, 174, 421, 422, 465, and 466 or amino acids 35-99 of SEQ ID NO: 18. In some embodiments, ALK2:MISRII heteromultimer complexes of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 50, 51, 75, 76, 79, 80, 133, 135, 161, 162, 419, 420, 457, and 458 or amino acids 24-116 of SEQ ID Nos: 50, 75, or 79. Preferably, ALK2:MISRII heteromultimer complexes of the present disclosure are soluble (e.g., comprise an extracellular domain of ALK2 and/or MISRII). In other preferred embodiments, ALK2:MISRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:MISRII heteromultimer complexes of the disclosure have different ligand binding specificities/profiles compared to their corresponding homomultimer complexes (i.e., ALK2 and MISRII homomultimers). ALK2:MISRII heteromultimers of the disclosure include, e.g., heterodimers, heterotrimers, heterotetramers and further oligomeric structures. In certain preferred embodiments, ALK2:MISRII heteromultimers of the disclosure are heterodimers.

In certain aspects, the disclosure relates to heteromultimers that comprise at least one ALK3 polypeptide, which includes fragments, functional variants, and modified forms thereof, and at least one MISRII polypeptide, which includes fragments, functional variants, and modified forms thereof. In some embodiments, ALK3:MISRII heteromultimers of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 22, 23, 115, 117, 175, 176, 407, 408, 467, and 468 or amino acids 61-130 of SEQ ID NO: 22. In some embodiments, ALK3:MISRII heteromultimer complexes of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 50, 51, 75, 76, 79, 80, 133, 135, 161, 162, 419, 420, 457, and 458 or amino acids 24-116 of SEQ ID Nos: 50, 75, or 79. Preferably, ALK3:MISRII heteromultimer complexes of the present disclosure are soluble (e.g., comprise an extracellular domain of ALK3 and/or MISRII). In other preferred embodiments, ALK3:MISRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK3:MISRII heteromultimer complexes of the disclosure have different ligand binding specificities/profiles compared to their corresponding homomultimer complexes (i.e., ALK3 and MISRII homomultimers). ALK3:MISRII heteromultimers of the disclosure include, e.g., heterodimers, heterotrimers, heterotetramers and further oligomeric structures. In certain preferred embodiments, ALK3:MISRII heteromultimers of the disclosure are heterodimers.

In certain aspects, the disclosure relates to heteromultimers that comprise at least one ALK4 polypeptide, which includes fragments, functional variants, and modified forms thereof, and at least one MISRII polypeptide, which includes fragments, functional variants, and modified forms thereof. In some embodiments, ALK4:MISRII heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 26, 27, 83, 84, 104, 106, 177, 178, 403, 404, 469, and 470 or amino acids 34-101 of SEQ ID NO: 26 or 83. In some embodiments, ALK4:MISRII heteromultimer complexes of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 50, 51, 75, 76, 79, 80, 133, 135, 161, 162, 419, 420, 457, and 458 or amino acids 24-116 of SEQ ID Nos: 50, 75, or 79. Preferably, ALK4:MISRII heteromultimer complexes of the present disclosure are soluble (e.g., comprise an extracellular domain of ALK4 and/or MISRII). In other preferred embodiments, ALK4:MISRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK4:MISRII heteromultimer complexes of the disclosure have different ligand binding specificities/profiles compared to their corresponding homomultimer complexes (i.e., ALK4 and MISRII homomultimers). ALK4:MISRII heteromultimers of the disclosure include, e.g., heterodimers, heterotrimers, heterotetramers and further oligomeric structures. In certain preferred embodiments, ALK4:MISRII heteromultimers of the disclosure are heterodimers.

In certain aspects, the disclosure relates to heteromultimers that comprise at least one ALK5 polypeptide, which includes fragments, functional variants, and modified forms thereof, and at least one MISRII polypeptide, which includes fragments, functional variants, and modified forms thereof. In some embodiments, ALK5:MISRII heteromultimers of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 30, 31, 87, 88, 139, 141, 179, 180, 423, 424, 471, and 472 or amino acids 36-106 of SEQ ID NOs: 30 or 87. In some embodiments, ALK5:MISRII heteromultimer complexes of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 50, 51, 75, 76, 79, 80, 133, 135, 161, 162, 419, 420, 457, and 458 or amino acids 24-116 of SEQ ID Nos: 50, 75, or 79. Preferably, ALK5:MISRII heteromultimer complexes of the present disclosure are soluble (e.g., comprise an extracellular domain of ALK5 and/or MISRII). In other preferred embodiments, ALK5:MISRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK5:MISRII heteromultimer complexes of the disclosure have different ligand binding specificities/profiles compared to their corresponding homomultimer complexes (i.e., ALK5 and MISRII homomultimers). ALK5:MISRII heteromultimers of the disclosure include, e.g., heterodimers, heterotrimers, heterotetramers and further oligomeric structures. In certain preferred embodiments, ALK5:MISRII heteromultimers of the disclosure are heterodimers.

In certain aspects, the disclosure relates to heteromultimers that comprise at least one ALK6 polypeptide, which includes fragments, functional variants, and modified forms thereof, and at least one MISRII polypeptide, which includes fragments, functional variants, and modified forms thereof. In some embodiments, ALK6:MISRII heteromultimers of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 34, 35, 91, 92, 142, 144, 181, 182, 425, 426, 473, and 474 or amino acids 32-102 of SEQ ID NO: 34 or amino acids 62-132 SEQ ID NO: 91. In some embodiments, ALK6:MISRII heteromultimer complexes of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 50, 51, 75, 76, 79, 80, 133, 135, 161, 162, 419, 420, 457, and 458 or amino acids 24-116 of SEQ ID Nos: 50, 75, or 79. Preferably, ALK6:MISRII heteromultimer complexes of the present disclosure are soluble (e.g., comprise an extracellular domain of ALK6 and/or MISRII). In other preferred embodiments, ALK6:MISRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK6:MISRII heteromultimer complexes of the disclosure have different ligand binding specificities/profiles compared to their corresponding homomultimer complexes (i.e., ALK6 and MISRII homomultimers). ALK6:MISRII heteromultimers of the disclosure include, e.g., heterodimers, heterotrimers, heterotetramers and further oligomeric structures. In certain preferred embodiments, ALK6:MISRII heteromultimers of the disclosure are heterodimers.

In certain aspects, the disclosure relates to heteromultimers that comprise at least one ALK7 polypeptide, which includes fragments, functional variants, and modified forms thereof, and at least one MISRII polypeptide, which includes fragments, functional variants, and modified forms thereof. In some embodiments, ALK7:MISRII heteromultimers of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 38, 39, 301, 302, 305, 306, 309, 310, 313, 112, 114, 183, 184, 405, 406, 475, and 476 or amino acids 28-93 of SEQ ID NOs: 38, 305, or 309. In some embodiments, ALK7:MISRII heteromultimer complexes of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 50, 51, 75, 76, 79, 80, 133, 135, 161, 162, 419, 420, 457, and 458 or amino acids 24-116 of SEQ ID Nos: 50, 75, or 79. Preferably, ALK7:MISRII heteromultimer complexes of the present disclosure are soluble (e.g., comprise an extracellular domain of ALK7 and/or MISRII). In other preferred embodiments, ALK7:MISRII heteromultimer complexes of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK7:MISRII heteromultimer complexes of the disclosure have different ligand binding specificities/profiles compared to their corresponding homomultimer complexes (i.e., ALK7 and MISRII homomultimers). ALK7:MISRII heteromultimers of the disclosure include, e.g., heterodimers, heterotrimers, heterotetramers and further oligomeric structures. In certain preferred embodiments, ALK7:MISRII heteromultimers of the disclosure are heterodimers.

In certain aspects, the disclosure relates to heteromultimers that comprise at least one ALK1 polypeptide, which includes fragments, functional variants, and modified forms thereof, and at least one BMPRII polypeptide, which includes fragments, functional variants, and modified forms thereof. In some embodiments, ALK1:BMPRII heteromultimer complexes of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:BMPRII heteromultimer complexes of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 46, 47, 71, 72, 121, 123, 155, 156, 411, 412, 455, and 456 or amino acids 34-123 of SEQ ID NO: 46 or 71. Preferably, ALK1:BMPRII heteromultimers of the present disclosure are soluble (e.g., comprise an extracellular domain of ALK1 and/or BMPRII). In other preferred embodiments, ALK1:BMPRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:BMPRII heteromultimers of the disclosure have different ligand binding specificities/profiles compared to their corresponding homomultimer complexes (i.e., ALK1 and BMPRII homomultimers). ALK1:BMPRII heteromultimer complexes of the disclosure include, e.g., heterodimers, heterotrimers, heterotetramers and further oligomeric structures. In certain preferred embodiments, ALK1:BMPRII heteromultimer complexes of the disclosure are heterodimers.

In certain aspects, the disclosure relates to heteromultimers that comprise at least one ALK2 polypeptide, which includes fragments, functional variants, and modified forms thereof, and at least one BMPRII polypeptide, which includes fragments, functional variants, and modified forms thereof. In some embodiments, ALK2:BMPRII heteromultimer complexes of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 18, 19, 136, 138, 173, 174, 421, 422, 465, and 466 or amino acids 35-99 of SEQ ID NO: 18. In some embodiments, ALK2:BMPRII heteromultimer complexes of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 46, 47, 71, 72, 121, 123, 155, 156, 411, 412, 455, and 456 or amino acids 34-123 of SEQ ID NO: 46 or 71. Preferably, ALK2:BMPRII heteromultimers of the present disclosure are soluble (e.g., comprise an extracellular domain of ALK2 and/or BMPRII). In other preferred embodiments, ALK2:BMPRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:BMPRII heteromultimers of the disclosure have different ligand binding specificities/profiles compared to their corresponding homomultimer complexes (i.e., ALK2 and BMPRII homomultimers). ALK2:BMPRII heteromultimer complexes of the disclosure include, e.g., heterodimers, heterotrimers, heterotetramers and further oligomeric structures. In certain preferred embodiments, ALK2:BMPRII heteromultimer complexes of the disclosure are heterodimers.

In certain aspects, the disclosure relates to heteromultimers that comprise at least one ALK3 polypeptide, which includes fragments, functional variants, and modified forms thereof, and at least one BMPRII polypeptide, which includes fragments, functional variants, and modified forms thereof. In some embodiments, ALK3:BMPRII heteromultimer complexes of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 22, 23, 115, 117, 175, 176, 407, 408, 467, and 468 or amino acids 61-130 of SEQ ID NO: 22. In some embodiments, ALK3:BMPRII heteromultimer complexes of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 46, 47, 71, 72, 121, 123, 155, 156, 411, 412, 455, and 456 or amino acids 34-123 of SEQ ID NO: 46 or 71. Preferably, ALK3:BMPRII heteromultimers of the present disclosure are soluble (e.g., comprise an extracellular domain of ALK3 and/or BMPRII). In other preferred embodiments, ALK3:BMPRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK3:BMPRII heteromultimers of the disclosure have different ligand binding specificities/profiles compared to their corresponding homomultimer complexes (i.e., ALK3 and BMPRII homomultimers). ALK3:BMPRII heteromultimer complexes of the disclosure include, e.g., heterodimers, heterotrimers, heterotetramers and further oligomeric structures. In certain preferred embodiments, ALK3:BMPRII heteromultimer complexes of the disclosure are heterodimers.

In certain aspects, the disclosure relates to heteromultimers that comprise at least one ALK4 polypeptide, which includes fragments, functional variants, and modified forms thereof, and at least one BMPRII polypeptide, which includes fragments, functional variants, and modified forms thereof. In some embodiments, ALK4:BMPRII heteromultimer complexes of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 26, 27, 83, 84, 104, 106, 177, 178, 403, 404, 469, and 470 or amino acids 34-101 of SEQ ID NOs: 26 or 83. In some embodiments, ALK4:BMPRII heteromultimer complexes of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 46, 47, 71, 72, 121, 123, 155, 156, 411, 412, 455, and 456 or amino acids 34-123 of SEQ ID NO: 46 or 71. Preferably, ALK4:BMPRII heteromultimers of the present disclosure are soluble (e.g., comprise an extracellular domain of ALK4 and/or BMPRII). In other preferred embodiments, ALK4:BMPRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK4:BMPRII heteromultimers of the disclosure have different ligand binding specificities/profiles compared to their corresponding homomultimer complexes (i.e., ALK4 and BMPRII homomultimers). ALK4:BMPRII heteromultimer complexes of the disclosure include, e.g., heterodimers, heterotrimers, heterotetramers and further oligomeric structures. In certain preferred embodiments, ALK4:BMPRII heteromultimer complexes of the disclosure are heterodimers.

In certain aspects, the disclosure relates to heteromultimers that comprise at least one ALK5 polypeptide, which includes fragments, functional variants, and modified forms thereof, and at least one BMPRII polypeptide, which includes fragments, functional variants, and modified forms thereof. In some embodiments, ALK5:BMPRII heteromultimer complexes of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: SEQ ID Nos: 30, 31, 87, 88, 139, 141, 179, 180, 423, 424, 471, and 472 or amino acids 36-106 of SEQ ID NOs: 30 or 87. In some embodiments, ALK5:BMPRII heteromultimer complexes of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 46, 47, 71, 72, 121, 123, 155, 156, 411, 412, 455, and 456 or amino acids 34-123 of SEQ ID NO: 46 or 71. Preferably, ALK5:BMPRII heteromultimers of the present disclosure are soluble (e.g., comprise an extracellular domain of ALK5 and/or BMPRII). In other preferred embodiments, ALK5:BMPRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK5:BMPRII heteromultimers of the disclosure have different ligand binding specificities/profiles compared to their corresponding homomultimer complexes (i.e., ALK5 and BMPRII homomultimers). ALK5:BMPRII heteromultimer complexes of the disclosure include, e.g., heterodimers, heterotrimers, heterotetramers and further oligomeric structures. In certain preferred embodiments, ALK5:BMPRII heteromultimer complexes of the disclosure are heterodimers.

In certain aspects, the disclosure relates to heteromultimers that comprise at least one ALK6 polypeptide, which includes fragments, functional variants, and modified forms thereof, and at least one BMPRII polypeptide, which includes fragments, functional variants, and modified forms thereof. In some embodiments, ALK6:BMPRII heteromultimer complexes of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 34, 35, 91, 92, 142, 144, 181, 182, 425, 426, 473, and 474 or amino acids 32-102 of SEQ ID NO: 34 or amino acids 62-132 of SEQ ID NO: 91. In some embodiments, ALK6:BMPRII heteromultimer complexes of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 46, 47, 71, 72, 121, 123, 155, 156, 411, 412, 455, and 456 or amino acids 34-123 of SEQ ID NO: 46 or 71. Preferably, ALK6:BMPRII heteromultimers of the present disclosure are soluble (e.g., comprise an extracellular domain of ALK6 and/or BMPRII). In other preferred embodiments, ALK6:BMPRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK6:BMPRII heteromultimers of the disclosure have different ligand binding specificities/profiles compared to their corresponding homomultimer complexes (i.e., ALK6 and BMPRII homomultimers). ALK6:BMPRII heteromultimer complexes of the disclosure include, e.g., heterodimers, heterotrimers, heterotetramers and further oligomeric structures. In certain preferred embodiments, ALK6:BMPRII heteromultimer complexes of the disclosure are heterodimers.

In certain aspects, the disclosure relates to heteromultimers that comprise at least one ALK7 polypeptide, which includes fragments, functional variants, and modified forms thereof, and at least one BMPRII polypeptide, which includes fragments, functional variants, and modified forms thereof. In some embodiments, ALK7:BMPRII heteromultimer complexes of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 38, 39, 301, 302, 305, 306, 309, 310, 313, 112, 114, 183, 184, 405, 406, 475, and 476 or amino acids 28-92 of SEQ ID NOs: 38, 305, or 309. In some embodiments, ALK7:BMPRII heteromultimer complexes of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 46, 47, 71, 72, 121, 123, 155, 156, 411, 412, 455, and 456 or amino acids 34-123 of SEQ ID NO: 46 or 71. Preferably, ALK7:BMPRII heteromultimers of the present disclosure are soluble (e.g., comprise an extracellular domain of ALK7 and/or BMPRII). In other preferred embodiments, ALK6:BMPRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK7:BMPRII heteromultimers of the disclosure have different ligand binding specificities/profiles compared to their corresponding homomultimer complexes (i.e., ALK7 and BMPRII homomultimers). ALK7:BMPRII heteromultimer complexes of the disclosure include, e.g., heterodimers, heterotrimers, heterotetramers and further oligomeric structures. In certain preferred embodiments, ALK7:BMPRII heteromultimer complexes of the disclosure are heterodimers.

In certain aspects, the disclosure relates to heteromultimers that comprise at least one ALK1 polypeptide, which includes fragments, functional variants, and modified forms thereof, and at least one ALK2 polypeptide, which includes fragments, functional variants, and modified forms thereof. In some embodiments, ALK1:ALK2 heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ALK2 heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 18, 19, 136, 138, 173, 174, 421, 422, 465, and 466 or amino acids 35-99 of SEQ ID NO: 18. Preferably, ALK1:ALK2 heteromultimers of the present disclosure are soluble (e.g., comprise an extracellular domain of ALK1 and/or ALK2). In other preferred embodiments, ALK1:ALK2 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ALK2 heteromultimers of the disclosure have different ligand binding specificities/profiles compared to their corresponding homomultimer complexes (i.e., ALK1 and ALK2 homomultimers). ALK1:ALK2 heteromultimers of the disclosure include, e.g., heterodimers, heterotrimers, heterotetramers and further oligomeric structures. In certain preferred embodiments, ALK1:ALK2 heteromultimers of the disclosure are heterodimers.

In certain aspects, the disclosure relates to heteromultimers that comprise at least one ALK1 polypeptide, which includes fragments, functional variants, and modified forms thereof, and at least one ALK2 polypeptide, which includes fragments, functional variants, and modified forms thereof. In some embodiments, ALK1:ALK2 heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ALK2 heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 18, 19, 136, 138, 173, 174, 421, 422, 465, and 466 or amino acids 35-99 of SEQ ID NO: 18. Preferably, ALK1:ALK2 heteromultimers of the present disclosure are soluble (e.g., comprise an extracellular domain of ALK1 and/or ALK2). In other preferred embodiments, ALK1:ALK2 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ALK2 heteromultimers of the disclosure have different ligand binding specificities/profiles compared to their corresponding homomultimer complexes (i.e., ALK1 and ALK2 homomultimers). ALK1:ALK2 heteromultimers of the disclosure include, e.g., heterodimers, heterotrimers, heterotetramers and further oligomeric structures. In certain preferred embodiments, ALK1:ALK2 heteromultimers of the disclosure are heterodimers.

In certain aspects, the disclosure relates to heteromultimers that comprise at least one ALK1 polypeptide, which includes fragments, functional variants, and modified forms thereof, and at least one ALK3 polypeptide, which includes fragments, functional variants, and modified forms thereof. In some embodiments, ALK1:ALK3 heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ALK3 heteromultimers of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 22, 23, 115, 117, 175, 176, 407, 408, 467, and 468 or amino acids 61-130 of SEQ ID NO: 22. Preferably, ALK1:ALK3 heteromultimers of the present disclosure are soluble (e.g., comprise an extracellular domain of ALK1 and/or ALK3). In other preferred embodiments, ALK1:ALK3 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ALK3 heteromultimers of the disclosure have different ligand binding specificities/profiles compared to their corresponding homomultimer complexes (i.e., ALK1 and ALK3 homomultimers). ALK1:ALK3 heteromultimers of the disclosure include, e.g., heterodimers, heterotrimers, heterotetramers and further oligomeric structures. In certain preferred embodiments, ALK1:ALK3 heteromultimers of the disclosure are heterodimers.

In certain aspects, the disclosure relates to heteromultimers that comprise at least one ALK1 polypeptide, which includes fragments, functional variants, and modified forms thereof, and at least one ALK4 polypeptide, which includes fragments, functional variants, and modified forms thereof. In some embodiments, ALK1:ALK4 heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ALK4 heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 26, 27, 83, 84, 104, 106, 177, 178, 403, 404, 469, and 470 or amino acids 34-101 of SEQ ID NOs: 26 or 83. Preferably, ALK1:ALK4 heteromultimers of the present disclosure are soluble (e.g., comprise an extracellular domain of ALK1 and/or ALK4). In other preferred embodiments, ALK1:ALK4 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ALK4 heteromultimers of the disclosure have different ligand binding specificities/profiles compared to their corresponding homomultimer complexes (i.e., ALK1 and ALK4 homomultimers). ALK1:ALK4 heteromultimers of the disclosure include, e.g., heterodimers, heterotrimers, heterotetramers and further oligomeric structures. In certain preferred embodiments, ALK1:ALK4 heteromultimers of the disclosure are heterodimers.

In certain aspects, the disclosure relates to heteromultimers that comprise at least one ALK1 polypeptide, which includes fragments, functional variants, and modified forms thereof, and at least one ALK4 polypeptide, which includes fragments, functional variants, and modified forms thereof. In some embodiments, ALK1:ALK4 heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ALK4 heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 26, 27, 83, 84, 104, 106, 177, 178, 403, 404, 469, and 470 or amino acids 34-101 of SEQ ID NOs: 26 or 83. Preferably, ALK1:ALK4 heteromultimers of the present disclosure are soluble (e.g., comprise an extracellular domain of ALK1 and/or ALK4). In other preferred embodiments, ALK1:ALK4 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ALK4 heteromultimers of the disclosure have different ligand binding specificities/profiles compared to their corresponding homomultimer complexes (i.e., ALK1 and ALK4 homomultimers). ALK1:ALK4 heteromultimers of the disclosure include, e.g., heterodimers, heterotrimers, heterotetramers and further oligomeric structures. In certain preferred embodiments, ALK1:ALK4 heteromultimers of the disclosure are heterodimers.

In certain aspects, the disclosure relates to heteromultimers that comprise at least one ALK1 polypeptide, which includes fragments, functional variants, and modified forms thereof, and at least one ALK5 polypeptide, which includes fragments, functional variants, and modified forms thereof. In some embodiments, ALK1:ALK5 heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ALK5 heteromultimers of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 30, 31, 87, 88, 139, 141, 179, 180, 423, 424, 471, and 472 or amino acids 36-106 of SEQ ID NOs: 30 or 87. Preferably, ALK1:ALK5 heteromultimers of the present disclosure are soluble (e.g., comprise an extracellular domain of ALK1 and/or ALK5). In other preferred embodiments, ALK1:ALK5 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ALK5 heteromultimers of the disclosure have different ligand binding specificities/profiles compared to their corresponding homomultimer complexes (i.e., ALK1 and ALK5 homomultimers). ALK1:ALK5 heteromultimers of the disclosure include, e.g., heterodimers, heterotrimers, heterotetramers and further oligomeric structures. In certain preferred embodiments, ALK1:ALK5 heteromultimers of the disclosure are heterodimers.

In certain aspects, the disclosure relates to heteromultimers that comprise at least one ALK1 polypeptide, which includes fragments, functional variants, and modified forms thereof, and at least one ALK6 polypeptide, which includes fragments, functional variants, and modified forms thereof. In some embodiments, ALK1:ALK6 heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ALK6 heteromultimers of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 34, 35, 91, 92, 142, 144, 181, 182, 425, 426, 473, and 474 or amino acids 32-102 of SEQ ID NO: 34 or amino acids 62-132 of SEQ ID NO: 91. Preferably, ALK1:ALK6 heteromultimers of the present disclosure are soluble (e.g., comprise an extracellular domain of ALK1 and/or ALK6). In other preferred embodiments, ALK1:ALK6 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ALK6 heteromultimers of the disclosure have different ligand binding specificities/profiles compared to their corresponding homomultimer complexes (i.e., ALK1 and ALK6 homomultimers). ALK1:ALK6 heteromultimers of the disclosure include, e.g., heterodimers, heterotrimers, heterotetramers and further oligomeric structures. In certain preferred embodiments, ALK1:ALK6 heteromultimers of the disclosure are heterodimers.

In certain aspects, the disclosure relates to heteromultimers that comprise at least one ALK1 polypeptide, which includes fragments, functional variants, and modified forms thereof, and at least one ALK7 polypeptide, which includes fragments, functional variants, and modified forms thereof. In some embodiments, ALK1:ALK7 heteromultimers of the disclosure comprise at least one ALK1 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID NO: 14, 15, 124, 126, 171, 172, 413, 414, 463, and 464 or amino acids 34-95 of SEQ ID NO: 14. In some embodiments, ALK1:ALK7 heteromultimers of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 38, 39, 301, 302, 305, 306, 309, 310, 313, 112, 114, 183, 184, 405, 406, 475, and 476 or amino acids 28-92 of SEQ ID NOs: 38, 305, or 309. Preferably, ALK1:ALK7 heteromultimers of the present disclosure are soluble (e.g., comprise an extracellular domain of ALK1 and/or ALK7). In other preferred embodiments, ALK1:ALK7 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK1:ALK7 heteromultimers of the disclosure have different ligand binding specificities/profiles compared to their corresponding homomultimer complexes (i.e., ALK1 and ALK7 homomultimers). ALK1:ALK7 heteromultimers of the disclosure include, e.g., heterodimers, heterotrimers, heterotetramers and further oligomeric structures. In certain preferred embodiments, ALK1:ALK7 heteromultimers of the disclosure are heterodimers.

In certain aspects, the disclosure relates to heteromultimers that comprise at least one ALK2 polypeptide, which includes fragments, functional variants, and modified forms thereof, and at least one ALK3 polypeptide, which includes fragments, functional variants, and modified forms thereof. In some embodiments, ALK2:ALK3 heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 18, 19, 136, 138, 173, 174, 421, 422, 465, and 466 or amino acids 35-99 of SEQ ID NO: 18. In some embodiments, ALK2:ALK3 heteromultimers of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 22, 23, 115, 117, 175, 176, 407, 408, 467, and 468 or amino acids 61-130 of SEQ ID NO: 22. Preferably, ALK2:ALK3 heteromultimers of the present disclosure are soluble (e.g., comprise an extracellular domain of ALK2 and/or ALK3). In other preferred embodiments, ALK2:ALK3 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:ALK3 heteromultimers of the disclosure have different ligand binding specificities/profiles compared to their corresponding homomultimer complexes (i.e., ALK2 and ALK3 homomultimers). ALK2:ALK3 heteromultimers of the disclosure include, e.g., heterodimers, heterotrimers, heterotetramers and further oligomeric structures. In certain preferred embodiments, ALK2:ALK3 heteromultimers of the disclosure are heterodimers.

In certain aspects, the disclosure relates to heteromultimers that comprise at least one ALK2 polypeptide, which includes fragments, functional variants, and modified forms thereof, and at least one ALK4 polypeptide, which includes fragments, functional variants, and modified forms thereof. In some embodiments, ALK2:ALK4 heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 18, 19, 136, 138, 173, 174, 421, 422, 465, and 466 or amino acids 35-99 of SEQ ID NO: 18. In some embodiments, ALK2:ALK4 heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 26, 27, 83, 84, 104, 106, 177, 178, 403, 404, 469, and 470 or amino acids 34-101 of SEQ ID NOs: 26 or 83. Preferably, ALK2:ALK4 heteromultimers of the present disclosure are soluble (e.g., comprise an extracellular domain of ALK2 and/or ALK4). In other preferred embodiments, ALK2:ALK4 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:ALK4 heteromultimers of the disclosure have different ligand binding specificities/profiles compared to their corresponding homomultimer complexes (i.e., ALK2 and ALK4 homomultimers). ALK2:ALK4 heteromultimers of the disclosure include, e.g., heterodimers, heterotrimers, heterotetramers and further oligomeric structures. In certain preferred embodiments, ALK2:ALK4 heteromultimers of the disclosure are heterodimers.

In certain aspects, the disclosure relates to heteromultimers that comprise at least one ALK2 polypeptide, which includes fragments, functional variants, and modified forms thereof, and at least one ALK5 polypeptide, which includes fragments, functional variants, and modified forms thereof. In some embodiments, ALK2:ALK5 heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 18, 19, 136, 138, 173, 174, 421, 422, 465, and 466 or amino acids 35-99 of SEQ ID NO: 18. In some embodiments, ALK2:ALK5 heteromultimers of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 30, 31, 87, 88, 139, 141, 179, 180, 423, 424, 471, and 472 or amino acids 36-106 of SEQ ID NOs: 30 or 87. Preferably, ALK2:ALK5 heteromultimers of the present disclosure are soluble (e.g., comprise an extracellular domain of ALK2 and/or ALK5). In other preferred embodiments, ALK2:ALK5 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:ALK5 heteromultimers of the disclosure have different ligand binding specificities/profiles compared to their corresponding homomultimer complexes (i.e., ALK2 and ALK5 homomultimers). ALK2:ALK5 heteromultimers of the disclosure include, e.g., heterodimers, heterotrimers, heterotetramers and further oligomeric structures. In certain preferred embodiments, ALK2:ALK5 heteromultimers of the disclosure are heterodimers.

In certain aspects, the disclosure relates to heteromultimers that comprise at least one ALK2 polypeptide, which includes fragments, functional variants, and modified forms thereof, and at least one ALK6 polypeptide, which includes fragments, functional variants, and modified forms thereof. In some embodiments, ALK2:ALK6 heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 18, 19, 136, 138, 173, 174, 421, 422, 465, and 466 or amino acids 35-99 of SEQ ID NO: 18. In some embodiments, ALK2:ALK6 heteromultimers of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 34, 35, 91, 92, 142, 144, 181, 182, 425, 426, 473, and 474 or amino acids 32-102 of SEQ ID NO: 34 or amino acids 62-132 of SEQ ID NO: 91. Preferably, ALK2:ALK6 heteromultimers of the present disclosure are soluble (e.g., comprise an extracellular domain of ALK2 and/or ALK6). In other preferred embodiments, ALK2:ALK6 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:ALK6 heteromultimers of the disclosure have different ligand binding specificities/profiles compared to their corresponding homomultimer complexes (i.e., ALK2 and ALK6 homomultimers). ALK2:ALK6 heteromultimers of the disclosure include, e.g., heterodimers, heterotrimers, heterotetramers and further oligomeric structures. In certain preferred embodiments, ALK2:ALK6 heteromultimers of the disclosure are heterodimers.

In certain aspects, the disclosure relates to heteromultimers that comprise at least one ALK2 polypeptide, which includes fragments, functional variants, and modified forms thereof, and at least one ALK7 polypeptide, which includes fragments, functional variants, and modified forms thereof. In some embodiments, ALK2:ALK7 heteromultimers of the disclosure comprise at least one ALK2 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 18, 19, 136, 138, 173, 174, 421, 422, 465, and 466 or amino acids 35-99 of SEQ ID NO: 18. In some embodiments, ALK2:ALK7 heteromultimers of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 38, 39, 301, 302, 305, 306, 309, 310, 313, 112, 114, 183, 184, 405, 406, 475, and 476 or amino acids 28-92 of SEQ ID NOs: 38, 305, or 309. Preferably, ALK2:ALK7 heteromultimers of the present disclosure are soluble (e.g., comprise an extracellular domain of ALK2 and/or ALK7). In other preferred embodiments, ALK2:ALK7 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK2:ALK7 heteromultimers of the disclosure have different ligand binding specificities/profiles compared to their corresponding homomultimer complexes (i.e., ALK2 and ALK7 homomultimers). ALK2:ALK7 heteromultimers of the disclosure include, e.g., heterodimers, heterotrimers, heterotetramers and further oligomeric structures. In certain preferred embodiments, ALK2:ALK7 heteromultimers of the disclosure are heterodimers.

In certain aspects, the disclosure relates to heteromultimers that comprise at least one ALK3 polypeptide, which includes fragments, functional variants, and modified forms thereof, and at least one ALK4 polypeptide, which includes fragments, functional variants, and modified forms thereof. In some embodiments, ALK3:ALK4 heteromultimers of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 22, 23, 115, 117, 175, 176, 407, 408, 467, and 468 or amino acids 61-130 of SEQ ID NO: 22. In some embodiments, ALK3:ALK4 heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 26, 27, 83, 84, 104, 106, 177, 178, 403, 404, 469, and 470 or amino acids 34-101 of SEQ ID NOs: 26 or 83. Preferably, ALK3:ALK4 heteromultimers of the present disclosure are soluble (e.g., comprise an extracellular domain of ALK3 and/or ALK4). In other preferred embodiments, ALK3:ALK4 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK3:ALK4 heteromultimers of the disclosure have different ligand binding specificities/profiles compared to their corresponding homomultimer complexes (i.e., ALK3 and ALK4 homomultimers). ALK3:ALK4 heteromultimers of the disclosure include, e.g., heterodimers, heterotrimers, heterotetramers and further oligomeric structures. In certain preferred embodiments, ALK3:ALK4 heteromultimers of the disclosure are heterodimers.

In certain aspects, the disclosure relates to heteromultimers that comprise at least one ALK3 polypeptide, which includes fragments, functional variants, and modified forms thereof, and at least one ALK5 polypeptide, which includes fragments, functional variants, and modified forms thereof. In some embodiments, ALK3:ALK5 heteromultimers of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 22, 23, 115, 117, 175, 176, 407, 408, 467, and 468 or amino acids 61-130 of SEQ ID NO: 22. In some embodiments, ALK3:ALK5 heteromultimers of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 30, 31, 87, 88, 139, 141, 179, 180, 423, 424, 471, and 472 or amino acids 36-106 of SEQ ID NOs: 30 or 87. Preferably, ALK3:ALK5 heteromultimers of the present disclosure are soluble (e.g., comprise an extracellular domain of ALK3 and/or ALK5). In other preferred embodiments, ALK3:ALK5 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK3:ALK5 heteromultimers of the disclosure have different ligand binding specificities/profiles compared to their corresponding homomultimer complexes (i.e., ALK3 and ALK5 homomultimers). ALK3:ALK5 heteromultimers of the disclosure include, e.g., heterodimers, heterotrimers, heterotetramers and further oligomeric structures. In certain preferred embodiments, ALK3:ALK5 heteromultimers of the disclosure are heterodimers.

In certain aspects, the disclosure relates to heteromultimers that comprise at least one ALK3 polypeptide, which includes fragments, functional variants, and modified forms thereof, and at least one ALK6 polypeptide, which includes fragments, functional variants, and modified forms thereof. In some embodiments, ALK3:ALK6 heteromultimers of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 22, 23, 115, 117, 175, 176, 407, 408, 467, and 468 or amino acids 61-130 of SEQ ID NO: 22. In some embodiments, ALK3:ALK6 heteromultimers of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 34, 35, 91, 92, 142, 144, 181, 182, 425, 426, 473, and 474 or amino acids 32-102 of SEQ ID NO: 34 or amino acids 62-132 of SEQ ID NO: 91. Preferably, ALK3:ALK6 heteromultimers of the present disclosure are soluble (e.g., comprise an extracellular domain of ALK3 and/or ALK6). In other preferred embodiments, ALK3:ALK6 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK3:ALK6 heteromultimers of the disclosure have different ligand binding specificities/profiles compared to their corresponding homomultimer complexes (i.e., ALK3 and ALK6 homomultimers). ALK3:ALK6 heteromultimers of the disclosure include, e.g., heterodimers, heterotrimers, heterotetramers and further oligomeric structures. In certain preferred embodiments, ALK3:ALK6 heteromultimers of the disclosure are heterodimers.

In certain aspects, the disclosure relates to heteromultimers that comprise at least one ALK3 polypeptide, which includes fragments, functional variants, and modified forms thereof, and at least one ALK7 polypeptide, which includes fragments, functional variants, and modified forms thereof. In some embodiments, ALK3:ALK7 heteromultimers of the disclosure comprise at least one ALK3 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 22, 23, 115, 117, 175, 176, 407, 408, 467, and 468 or amino acids 61-130 of SEQ ID NO: 22. In some embodiments, ALK3:ALK7 heteromultimers of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 38, 39, 301, 302, 305, 306, 309, 310, 313, 112, 114, 183, 184, 405, 406, 475, and 476 or amino acids 28-92 of SEQ ID NOs: 38, 305, or 309. Preferably, ALK3:ALK7 heteromultimers of the present disclosure are soluble (e.g., comprise an extracellular domain of ALK3 and/or ALK7). In other preferred embodiments, ALK3:ALK7 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK3:ALK7 heteromultimers of the disclosure have different ligand binding specificities/profiles compared to their corresponding homomultimer complexes (i.e., ALK3 and ALK7 homomultimers). ALK3:ALK7 heteromultimers of the disclosure include, e.g., heterodimers, heterotrimers, heterotetramers and further oligomeric structures. In certain preferred embodiments, ALK3:ALK7 heteromultimers of the disclosure are heterodimers.

In certain aspects, the disclosure relates to heteromultimers that comprise at least one ALK4 polypeptide, which includes fragments, functional variants, and modified forms thereof, and at least one ALK5 polypeptide, which includes fragments, functional variants, and modified forms thereof. In some embodiments, ALK4:ALK5 heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 26, 27, 83, 84, 104, 106, 177, 178, 403, 404, 469, and 470 or amino acids 34-101 of SEQ ID NOs: 26 or 83. In some embodiments, ALK4:ALK5 heteromultimers of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 30, 31, 87, 88, 139, 141, 179, 180, 423, 424, 471, and 472 or amino acids 36-106 of SEQ ID NOs: 30 or 87. Preferably, ALK4:ALK5 heteromultimers of the present disclosure are soluble (e.g., comprise an extracellular domain of ALK4 and/or ALK5). In other preferred embodiments, ALK4:ALK5 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK4:ALK5 heteromultimers of the disclosure have different ligand binding specificities/profiles compared to their corresponding homomultimer complexes (i.e., ALK4 and ALK5 homomultimers). ALK4:ALK5 heteromultimers of the disclosure include, e.g., heterodimers, heterotrimers, heterotetramers and further oligomeric structures. In certain preferred embodiments, ALK4:ALK5 heteromultimers of the disclosure are heterodimers.

In certain aspects, the disclosure relates to heteromultimers that comprise at least one ALK4 polypeptide, which includes fragments, functional variants, and modified forms thereof, and at least one ALK6 polypeptide, which includes fragments, functional variants, and modified forms thereof. In some embodiments, ALK4:ALK6 heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 26, 27, 83, 84, 104, 106, 177, 178, 403, 404, 469, and 470 or amino acids 34-101 of SEQ ID NOs: 26 or 83. In some embodiments, ALK4:ALK6 heteromultimers of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 34, 35, 91, 92, 142, 144, 181, 182, 425, 426, 473, and 474 or amino acids 32-102 of SEQ ID NO: 34 or amino acids 62-132 of SEQ ID NO: 91. Preferably, ALK4:ALK6 heteromultimers of the present disclosure are soluble (e.g., comprise an extracellular domain of ALK4 and/or ALK6). In other preferred embodiments, ALK4:ALK6 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK4:ALK6 heteromultimers of the disclosure have different ligand binding specificities/profiles compared to their corresponding homomultimer complexes (i.e., ALK4 and ALK6 homomultimers). ALK4:ALK6 heteromultimers of the disclosure include, e.g., heterodimers, heterotrimers, heterotetramers and further oligomeric structures. In certain preferred embodiments, ALK4:ALK6 heteromultimers of the disclosure are heterodimers.

In certain aspects, the disclosure relates to heteromultimers that comprise at least one ALK4 polypeptide, which includes fragments, functional variants, and modified forms thereof, and at least one ALK7 polypeptide, which includes fragments, functional variants, and modified forms thereof. In some embodiments, ALK4:ALK7 heteromultimers of the disclosure comprise at least one ALK4 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 26, 27, 83, 84, 104, 106, 177, 178, 403, 404, 469, and 470 or amino acids 34-101 of SEQ ID NOs: 26 or 83. In some embodiments, ALK4:ALK7 heteromultimers of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 38, 39, 301, 302, 305, 306, 309, 310, 313, 112, 114, 183, 184, 405, 406, 475, and 476 or amino acids 28-92 of SEQ ID NOs: 38, 305, or 309. Preferably, ALK4:ALK7 heteromultimers of the present disclosure are soluble (e.g., comprise an extracellular domain of ALK4 and/or ALK7). In other preferred embodiments, ALK4:ALK7 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK4:ALK7 heteromultimers of the disclosure have different ligand binding specificities/profiles compared to their corresponding homomultimer complexes (i.e., ALK4 and ALK7 homomultimers). ALK4:ALK7 heteromultimers of the disclosure include, e.g., heterodimers, heterotrimers, heterotetramers and further oligomeric structures. In certain preferred embodiments, ALK4:ALK7 heteromultimers of the disclosure are heterodimers.

In certain aspects, the disclosure relates to heteromultimers that comprise at least one ALK5 polypeptide, which includes fragments, functional variants, and modified forms thereof, and at least one ALK6 polypeptide, which includes fragments, functional variants, and modified forms thereof. In some embodiments, ALK5:ALK6 heteromultimers of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 30, 31, 87, 88, 139, 141, 179, 180, 423, 424, 471, and 472 or amino acids 36-106 of SEQ ID NOs: 30 or 87. In some embodiments, ALK5:ALK6 heteromultimers of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 34, 35, 91, 92, 142, 144, 181, 182, 425, 426, 473, and 474 or amino acids 32-102 of SEQ ID NO: 34 or amino acids 62-132 of SEQ ID NO: 91. Preferably, ALK5:ALK6 heteromultimers of the present disclosure are soluble (e.g., comprise an extracellular domain of ALK5 and/or ALK6). In other preferred embodiments, ALK5:ALK6 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK5:ALK6 heteromultimers of the disclosure have different ligand binding specificities/profiles compared to their corresponding homomultimer complexes (i.e., ALK5 and ALK6 homomultimers). ALK5:ALK6 heteromultimers of the disclosure include, e.g., heterodimers, heterotrimers, heterotetramers and further oligomeric structures. In certain preferred embodiments, ALK5:ALK6 heteromultimers of the disclosure are heterodimers.

In certain aspects, the disclosure relates to heteromultimers that comprise at least one ALK5 polypeptide, which includes fragments, functional variants, and modified forms thereof, and at least one ALK7 polypeptide, which includes fragments, functional variants, and modified forms thereof. In some embodiments, ALK5:ALK7 heteromultimers of the disclosure comprise at least one ALK5 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 30, 31, 87, 88, 139, 141, 179, 180, 423, 424, 471, and 472 or amino acids 36-106 of SEQ ID NOs: 30 or 87. In some embodiments, ALK5:ALK7 heteromultimers of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 38, 39, 301, 302, 305, 306, 309, 310, 313, 112, 114, 183, 184, 405, 406, 475, and 476 or amino acids 28-92 of SEQ ID NOs: 38, 305, or 309. Preferably, ALK5:ALK7 heteromultimers of the present disclosure are soluble (e.g., comprise an extracellular domain of ALK5 and/or ALK7). In other preferred embodiments, ALK5:ALK7 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK5:ALK7 heteromultimers of the disclosure have different ligand binding specificities/profiles compared to their corresponding homomultimer complexes (i.e., ALK5 and ALK7 homomultimers). ALK5:ALK7 heteromultimers of the disclosure include, e.g., heterodimers, heterotrimers, heterotetramers and further oligomeric structures. In certain preferred embodiments, ALK5:ALK7 heteromultimers of the disclosure are heterodimers.

In certain aspects, the disclosure relates to heteromultimers that comprise at least one ALK6 polypeptide, which includes fragments, functional variants, and modified forms thereof, and at least one ALK7 polypeptide, which includes fragments, functional variants, and modified forms thereof. In some embodiments, ALK6:ALK7 heteromultimers of the disclosure comprise at least one ALK6 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 34, 35, 91, 92, 142, 144, 181, 182, 425, 426, 473, and 474 or amino acids 32-102 of SEQ ID NO: 34 or amino acids 62-132 of SEQ ID NO: 91. In some embodiments, ALK6:ALK7 heteromultimers of the disclosure comprise at least one ALK7 polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 38, 39, 301, 302, 305, 306, 309, 310, 313, 112, 114, 183, 184, 405, 406, 475, and 476 or amino acids 28-92 of SEQ ID NOs: 38, 305, or 309. Preferably, ALK5:ALK7 heteromultimers of the present disclosure are soluble (e.g., comprise an extracellular domain of ALK6 and/or ALK7). In other preferred embodiments, ALK6:ALK7 heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ALK6:ALK7 heteromultimers of the disclosure have different ligand binding specificities/profiles compared to their corresponding homomultimer complexes (i.e., ALK6 and ALK7 homomultimers). ALK6:ALK7 heteromultimers of the disclosure include, e.g., heterodimers, heterotrimers, heterotetramers and further oligomeric structures. In certain preferred embodiments, ALK6:ALK7 heteromultimers of the disclosure are heterodimers.

In certain aspects, the disclosure relates to heteromultimers that comprise at least one ActRIIA polypeptide, which includes fragments, functional variants, and modified forms thereof, and at least one ActRIIB polypeptide, which includes fragments, functional variants, and modified forms thereof. In some embodiments, ActRIIA:ActRIIB heteromultimers of the disclosure comprise at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 9, 10, 11, 118, 120, 151, 152, 409, 410, 451, and 452 or amino acids 30-110 of SEQ ID NO: 9. In some embodiments, ActRIIA:ActRIIB heteromultimers of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 1, 2, 3, 4, 5, 6, 100, 102, 153, 154, 401, 402, 453, and 454 or amino acids 29-109 of SEQ ID NO: 1 or 25-131 of SEQ ID NO: 1. Preferably, ActRIIA:ActRIIB heteromultimers of the present disclosure are soluble (e.g., comprise an extracellular domain of ActRIIA and/or ActRIIB). In other preferred embodiments, ActRIIA:ActRIIB heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ActRIIA:ActRIIB heteromultimers of the disclosure have different ligand binding specificities/profiles compared to their corresponding homomultimer complexes (i.e., ActRIIA and ActRIIB homomultimers). ActRIIA:ActRIIB heteromultimers of the disclosure include, e.g., heterodimers, heterotrimers, heterotetramers and further oligomeric structures. In certain preferred embodiments, ActRIIA:ActRIIB heteromultimers of the disclosure are heterodimers.

In certain aspects, the disclosure relates to heteromultimers that comprise at least one ActRIIA polypeptide, which includes fragments, functional variants, and modified forms thereof, and at least one BMPRII polypeptide, which includes fragments, functional variants, and modified forms thereof. In some embodiments, ActRIIA:BMPRII heteromultimers of the disclosure comprise at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 9, 10, 11, 118, 120, 151, 152, 409, 410, 451, and 452 or amino acids 30-110 of SEQ ID NO: 9. In some embodiments, ActRIIA:BMPRII heteromultimers of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 46, 47, 71, 72, 121, 123, 155, 156, 411, 412, 455, and 456 or amino acids 34-123 of SEQ ID NO: 46 and 71. Preferably, ActRIIA:BMPRII heteromultimers of the present disclosure are soluble (e.g., comprise an extracellular domain of ActRIIA and/or BMPRII). In other preferred embodiments, ActRIIA:BMPRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ActRIIA:BMPRII heteromultimers of the disclosure have different ligand binding specificities/profiles compared to their corresponding homomultimer complexes (i.e., ActRIIA and BMPRII homomultimers). ActRIIA:BMPRII heteromultimers of the disclosure include, e.g., heterodimers, heterotrimers, heterotetramers and further oligomeric structures. In certain preferred embodiments, ActRIIA:BMPRII heteromultimers of the disclosure are heterodimers.

In certain aspects, the disclosure relates to heteromultimers that comprise at least one ActRIIA polypeptide, which includes fragments, functional variants, and modified forms thereof, and at least one TGFBRII polypeptide, which includes fragments, functional variants, and modified forms thereof. In some embodiments, ActRIIA:TGFBRII heteromultimers of the disclosure comprise at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 9, 10, 11, 118, 120, 151, 152, 409, 410, 451, and 452 or amino acids 30-110 of SEQ ID NO: 9. In some embodiments, ActRIIA:TGFBRII heteromultimers of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, ActRIIA:TGFBRII heteromultimers of the present disclosure are soluble (e.g., comprise an extracellular domain of ActRIIA and/or BMPRII). In other preferred embodiments, ActRIIA:TGFBRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ActRIIA:TGFBRII heteromultimers of the disclosure have different ligand binding specificities/profiles compared to their corresponding homomultimer complexes (i.e., ActRIIA and TGFBRII homomultimers). ActRIIA:TGFBRII heteromultimers of the disclosure include, e.g., heterodimers, heterotrimers, heterotetramers and further oligomeric structures. In certain preferred embodiments, ActRIIA:TGFBRII heteromultimers of the disclosure are heterodimers.

In certain aspects, the disclosure relates to heteromultimers that comprise at least one ActRIIA polypeptide, which includes fragments, functional variants, and modified forms thereof, and at least one MISRII polypeptide, which includes fragments, functional variants, and modified forms thereof. In some embodiments, ActRIIA:MISRII heteromultimers of the disclosure comprise at least one ActRIIA polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 9, 10, 11, 118, 120, 151, 152, 409, 410, 451, and 452 or amino acids 30-110 of SEQ ID NO: 9. In some embodiments, ActRIIA:MISRII heteromultimers of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 50, 51, 75, 76, 79, 80, 133, 135, 161, 162, 419, 420, 457, and 458 or amino acids 24-116 of SEQ ID NO: 50, 75, and 79. Preferably, ActRIIA:MISRII heteromultimers of the present disclosure are soluble (e.g., comprise an extracellular domain of ActRIIA and/or MISRII). In other preferred embodiments, ActRIIA:MISRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ActRIIA:MISRII heteromultimers of the disclosure have different ligand binding specificities/profiles compared to their corresponding homomultimer complexes (i.e., ActRIIA and MISRII homomultimers). ActRIIA:MISRII heteromultimers of the disclosure include, e.g., heterodimers, heterotrimers, heterotetramers and further oligomeric structures. In certain preferred embodiments, ActRIIA:MISRII heteromultimers of the disclosure are heterodimers.

In certain aspects, the disclosure relates to heteromultimers that comprise at least one ActRIIB polypeptide, which includes fragments, functional variants, and modified forms thereof, and at least one BMPRII polypeptide, which includes fragments, functional variants, and modified forms thereof. In some embodiments, ActRIIB:BMPRII heteromultimers of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 1, 2, 3, 4, 5, 6, 100, 102, 153, 154, 401, 402, 453, and 454 or amino acids 29-109 of SEQ ID NO: 1 or 25-131 of SEQ ID NO: 1. In some embodiments, ActRIIB:BMPRII heteromultimers of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 46, 47, 71, 72, 121, 123, 155, 156, 411, 412, 455, and 456 or amino acids 34-123 of SEQ ID NO: 46 and 71. Preferably, ActRIIB:BMPRII heteromultimers of the present disclosure are soluble (e.g., comprise an extracellular domain of ActRIIB and/or BMPRII). In other preferred embodiments, ActRIIB:BMPRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ActRIIB:BMPRII heteromultimers of the disclosure have different ligand binding specificities/profiles compared to their corresponding homomultimer complexes (i.e., ActRIIB and BMPRII homomultimers). ActRIIB:BMPRII heteromultimers of the disclosure include, e.g., heterodimers, heterotrimers, heterotetramers and further oligomeric structures. In certain preferred embodiments, ActRIIB:BMPRII heteromultimers of the disclosure are heterodimers.

In certain aspects, the disclosure relates to heteromultimers that comprise at least one ActRIIB polypeptide, which includes fragments, functional variants, and modified forms thereof, and at least one TGFBRII polypeptide, which includes fragments, functional variants, and modified forms thereof. In some embodiments, ActRIIB:TGFBRII heteromultimers of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 1, 2, 3, 4, 5, 6, 100, 102, 153, 154, 401, 402, 453, and 454 or amino acids 29-109 of SEQ ID NO: 1 or 25-131 of SEQ ID NO: 1. In some embodiments, ActRIIB:TGFBRII heteromultimers of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, ActRIIB:TGFBRII heteromultimers of the present disclosure are soluble (e.g., comprise an extracellular domain of ActRIIB and/or TGFβRII). In other preferred embodiments, ActRIIB:TGFBRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ActRIIB:TGFBRII heteromultimers of the disclosure have different ligand binding specificities/profiles compared to their corresponding homomultimer complexes (i.e., ActRIIB and TGFBRII homomultimers). ActRIIB:TGFBRII heteromultimers of the disclosure include, e.g., heterodimers, heterotrimers, heterotetramers and further oligomeric structures. In certain preferred embodiments, ActRIIB:TGFBRII heteromultimers of the disclosure are heterodimers.

In certain aspects, the disclosure relates to heteromultimers that comprise at least one ActRIIB polypeptide, which includes fragments, functional variants, and modified forms thereof, and at least one MISRII polypeptide, which includes fragments, functional variants, and modified forms thereof. In some embodiments, ActRIIB:MISRII heteromultimers of the disclosure comprise at least one ActRIIB polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 1, 2, 3, 4, 5, 6, 100, 102, 153, 154, 401, 402, 453, and 454 or amino acids 29-109 of SEQ ID NO: 1 or 25-131 of SEQ ID NO: 1. In some embodiments, ActRIIB:MISRII heteromultimers of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 50, 51, 75, 76, 79, 80, 133, 135, 161, 162, 419, 420, 457, and 458 or amino acids 24-116 of SEQ ID NO: 50, 75, and 79. Preferably, ActRIIB:MISRII heteromultimers of the present disclosure are soluble (e.g., comprise an extracellular domain of ActRIIB and/or MISRII). In other preferred embodiments, ActRIIB:MISRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, ActRIIB:MISRII heteromultimers of the disclosure have different ligand binding specificities/profiles compared to their corresponding homomultimer complexes (i.e., ActRIIB and MISRII homomultimers). ActRIIB:MISRII heteromultimers of the disclosure include, e.g., heterodimers, heterotrimers, heterotetramers and further oligomeric structures. In certain preferred embodiments, ActRIIB:MISRII heteromultimers of the disclosure are heterodimers.

In certain aspects, the disclosure relates to heteromultimers that comprise at least one BMPRII polypeptide, which includes fragments, functional variants, and modified forms thereof, and at least one TGFBRII polypeptide, which includes fragments, functional variants, and modified forms thereof. In some embodiments, BMPRII:TGFBRII heteromultimers of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 46, 47, 71, 72, 121, 123, 155, 156, 411, 412, 455, and 456 or amino acids 34-123 of SEQ ID NO: 46 or 71. In some embodiments, BMPRII:TGFBRII heteromultimers of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. Preferably, BMPRII:TGFBRII heteromultimers of the present disclosure are soluble (e.g., comprise an extracellular domain of BMPRII and/or TGFBRII). In other preferred embodiments, BMPRII:TGFBRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, BMPRII:TGFBRII heteromultimers of the disclosure have different ligand binding specificities/profiles compared to their corresponding homomultimer complexes (i.e., BMPRII and TGFBRII homomultimers). BMPRII:TGFBRII heteromultimers of the disclosure include, e.g., heterodimers, heterotrimers, heterotetramers and further oligomeric structures. In certain preferred embodiments, BMPRII:TGFBRII heteromultimers of the disclosure are heterodimers.

In certain aspects, the disclosure relates to heteromultimers that comprise at least one BMPRII polypeptide, which includes fragments, functional variants, and modified forms thereof, and at least one MISRII polypeptide, which includes fragments, functional variants, and modified forms thereof. In some embodiments, BMPRII:MISRII heteromultimers of the disclosure comprise at least one BMPRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 46, 47, 71, 72, 121, 123, 155, 156, 411, 412, 455, and 456 or amino acids 34-123 of SEQ ID NO: 46 or 71. In some embodiments, BMPRII:MISRII heteromultimers of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 50, 51, 75, 76, 79, 80, 133, 135, 161, 162, 419, 420, 457, and 458 or amino acids 24-116 of SEQ ID NO: 50, 75, or 79. Preferably, BMPRII:MISRII heteromultimers of the present disclosure are soluble (e.g., comprise an extracellular domain of BMPRII and/or MISRII). In other preferred embodiments, BMPRII:MISRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, BMPRII:MISRII heteromultimers of the disclosure have different ligand binding specificities/profiles compared to their corresponding homomultimer complexes (i.e., BMPRII and MISRII homomultimers). BMPRII:MISRII heteromultimers of the disclosure include, e.g., heterodimers, heterotrimers, heterotetramers and further oligomeric structures. In certain preferred embodiments, BMPRII:MISRII heteromultimers of the disclosure are heterodimers.

In certain aspects, the disclosure relates to heteromultimers that comprise at least one TGFBRII polypeptide, which includes fragments, functional variants, and modified forms thereof, and at least one MISRII polypeptide, which includes fragments, functional variants, and modified forms thereof. In some embodiments, TGFBRII:MISRII heteromultimers of the disclosure comprise at least one TGFBRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 42, 43, 67, 68, 127, 129, 130, 132, 157, 158, 159, 160, 415, 416, 417, 418, 459, 460, 461, and 462 or amino acids 44-168 of SEQ ID NO: 67 or amino acids 51-143 of SEQ ID NO: 42. In some embodiments, TGFBRII:MISRII heteromultimers of the disclosure comprise at least one MISRII polypeptide that comprises, consists, or consists essentially of a sequence that is at least 70%, 75%, 80%, 85%, 90%, 91%, 92%, 93%, 94%, 95%, 96% 97%, 98%, 99%, or 100% identical to the amino acid sequence of any one of SEQ ID Nos: 50, 51, 75, 76, 79, 80, 133, 135, 161, 162, 419, 420, 457, and 458 or amino acids 24-116 of SEQ ID NO: 50, 75, or 79. Preferably, TGFBRII:MISRII heteromultimers of the present disclosure are soluble (e.g., comprise an extracellular domain of TGFBII and/or MISRII). In other preferred embodiments, TGFBRII:MISRII heteromultimers of the disclosure bind to and/or inhibit (antagonize) activity (e.g., induction of Smad 2/3 and/or Smad 1/5/8 signaling) of one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). In some embodiments, TGFBRII:MISRII heteromultimers of the disclosure have different ligand binding specificities/profiles compared to their corresponding homomultimer complexes (i.e., TGFβRII and MISRII homomultimers). TGFBRII:MISRII heteromultimers of the disclosure include, e.g., heterodimers, heterotrimers, heterotetramers and further oligomeric structures. In certain preferred embodiments, TGFBRII:MISRII heteromultimers of the disclosure are heterodimers.

In some embodiments, the present disclosure contemplates making functional variants by modifying the structure of a TGF-beta superfamily type I receptor polypeptide (e.g., ALK1, ALK2, ALK3, ALK4, ALK5, ALK6, and ALK7) and/or a TGF-beta superfamily type II receptor polypeptide (e.g., ActRIIA, ActRIIB, TGFBRII, BMPRII, and MISRII) for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more TGF-beta superfamily ligands including, for example, BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty.

In some embodiments, the present disclosure contemplates making functional variants by modifying the structure of the TGF-beta superfamily type I receptor polypeptide and/or TGF-beta superfamily type II receptor polypeptide for such purposes as enhancing therapeutic efficacy or stability (e.g., increased shelf-life and/or increased resistance to proteolytic degradation).

In certain embodiments, the present disclosure contemplates specific mutations of a TGF-beta superfamily type I receptor polypeptide (e.g., ALK1, ALK2, ALK3, ALK4, ALK5, ALK6, and ALK7) and/or a TGF-beta superfamily type II receptor polypeptide (e.g., ActRIIA, ActRIIB, TGFBRII, BMPRII, and MISRII) receptor of the disclosure so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, TGF-beta superfamily type I and II receptor complexes of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of a TGF-beta superfamily type I receptor polypeptide (e.g., ALK1, ALK2, ALK3, ALK4, ALK5, ALK6, and ALK7) and/or a TGF-beta superfamily type II receptor polypeptide (e.g., ActRIIA, ActRIIB, TGFBRII, BMPRII, and MISRII) of the present disclosure, as well as truncation mutants. Pools of combinatorial mutants are especially useful for identifying functionally active (e.g., ligand binding) TGF-beta superfamily type I and/or TGF-beta superfamily type II receptor sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, TGF-beta superfamily type I and II receptor complex variants may be screened for ability to bind to a TGF-beta superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty), to prevent binding of a TGF-beta superfamily ligand to a TGF-beta superfamily receptor, and/or to interfere with signaling caused by an TGF-beta superfamily ligand.

The activity of a TGF-beta superfamily heteromultimer complex of the disclosure also may be tested, for example in a cell-based or in vivo assay. For example, the effect of a heteromultimer complex on the expression of genes or the activity of proteins involved in muscle production in a muscle cell may be assessed. This may, as needed, be performed in the presence of one or more recombinant TGF-beta superfamily ligand proteins (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty), and cells may be transfected so as to produce a TGF-beta superfamily type I and II receptor complex, and optionally, a TGF-beta superfamily ligand. Likewise, a heteromultimer complex of the disclosure may be administered to a mouse or other animal, and one or more measurements, such as muscle formation and strength may be assessed using art-recognized methods. Similarly, the activity of a heteromultimer, or variants thereof, may be tested in osteoblasts, adipocytes, and/or neuronal cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference TGF-beta superfamily heteromultimer complex. Such variants, when expressed from recombinant DNA constructs, can be used in gene therapy protocols. Likewise, mutagenesis can give rise to variants which have intracellular half-lives dramatically different than the corresponding unmodified TGF-beta superfamily heteromultimer complex. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter one or more activities of the TGF-beta superfamily heteromultimer complex including, for example, immunogenicity, half-life, and solubility.

A combinatorial library may be produced by way of a degenerate library of genes encoding a library of polypeptides which each include at least a portion of potential TGF-beta superfamily type I and/or II receptor sequences. For instance, a mixture of synthetic oligonucleotides can be enzymatically ligated into gene sequences such that the degenerate set of potential TGF-beta superfamily type I and/or II receptor encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art. See, e.g., Narang, SA (1983) Tetrahedron 39:3; Itakura et al. (1981) Recombinant DNA, Proc. 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins. See, e.g., Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815.

Alternatively, other forms of mutagenesis can be utilized to generate a combinatorial library. For example, TGF-beta superfamily heteromultimer complexes of the disclosure can be generated and isolated from a library by screening using, for example, alanine scanning mutagenesis [see, e.g., Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [see, e.g., Gustin et al. (1993) Virology 193:653-660; and Brown et al. (1992) Mol. Cell Biol. 12:2644-2652; McKnight et al. (1982) Science 232:316], by saturation mutagenesis [see, e.g., Meyers et al., (1986) Science 232:613]; by PCR mutagenesis [see, e.g., Leung et al. (1989) Method Cell Mol Biol 1:11-19]; or by random mutagenesis, including chemical mutagenesis [see, e.g., Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of TGF-beta superfamily type I and/or II receptor polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of TGF-beta superfamily heteromultimer complexes of the disclosure. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include TGF-beta superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty) binding assays and/or TGF-beta superfamily ligand-mediated cell signaling assays.

In certain embodiments, TGF-beta superfamily type I and II heteromultimer complexes of the disclosure may further comprise post-translational modifications in addition to any that are naturally present in the TGF-beta superfamily type I and/or II receptor polypeptide. Such modifications include, but are not limited to, acetylation, carboxylation, glycosylation, phosphorylation, lipidation, and acylation. As a result, the TGF-beta superfamily type I and II heteromultimer complex may comprise non-amino acid elements, such as polyethylene glycols, lipids, polysaccharide or monosaccharide, and phosphates. Effects of such non-amino acid elements on the functionality of a heteromultimer complex may be tested as described herein for other heteromultimer complex variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the TGF-beta superfamily type I and/or type II receptor polypeptides as well as heteromultimers comprising the same.

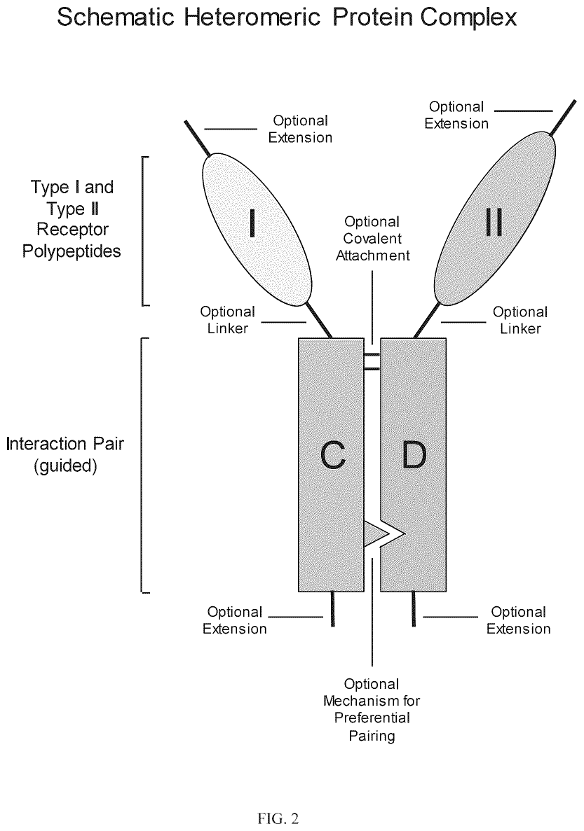

In certain aspects, the polypeptides disclosed herein may form protein complexes comprising at least one TGF-beta superfamily type I polypeptide associated, covalently or non-covalently, with at least one type II receptor polypeptide. Preferably, polypeptides disclosed herein form heterodimeric complexes, although higher order heteromultimeric complexes (heteromultimers) are also included such as, but not limited to, heterotrimers, heterotetramers, and further oligomeric structures (see, e.g., FIGS. 1, 2, and 15-17). In some embodiments, TGF-beta superfamily type I and/or type II receptor polypeptides of the present disclosure comprise at least one multimerization domain. As disclosed herein, the term “multimerization domain” refers to an amino acid or sequence of amino acids that promote covalent or non-covalent interaction between at least a first polypeptide and at least a second polypeptide. Polypeptides disclosed herein may be joined covalently or non-covalently to a multimerization domain. Preferably, a multimerization domain promotes interaction between a first polypeptide (e.g., TGF-beta superfamily type I polypeptide) and a second polypeptide (e.g., TGF-beta superfamily type II polypeptide) to promote heteromultimer formation (e.g., heterodimer formation), and optionally hinders or otherwise disfavors homomultimer formation (e.g., homodimer formation), thereby increasing the yield of desired heteromultimer (see, e.g., FIG. 2).

Many methods known in the art can be used to generate TGF-beta superfamily complexes of the disclosure. For example, non-naturally occurring disulfide bonds may be constructed by replacing on a first polypeptide (e.g., TGF-beta superfamily type I polypeptide) a naturally occurring amino acid with a free thiol-containing residue, such as cysteine, such that the free thiol interacts with another free thiol-containing residue on a second polypeptide (e.g., TGF-beta superfamily type II polypeptide) such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S. Pat. No. 8,592,562; coiled-coil interactions such as described in Christensen et al., U.S. 20120302737; leucine zippers such as described in Pack & Plueckthun, (1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

In certain aspects, a multimerization domain may comprise one component of an interaction pair. In some embodiments, the polypeptides disclosed herein may form protein complexes comprising a first polypeptide covalently or non-covalently associated with a second polypeptide, wherein the first polypeptide comprises the amino acid sequence of a TGF-beta superfamily type I polypeptide and the amino acid sequence of a first member of an interaction pair; and the second polypeptide comprises the amino acid sequence of a TGF-beta superfamily type II polypeptide and the amino acid sequence of a second member of an interaction pair. The interaction pair may be any two polypeptide sequences that interact to form a complex, particularly a heterodimeric complex although operative embodiments may also employ an interaction pair that can form a homodimeric complex. One member of the interaction pair may be fused to a TGF-beta superfamily type I or type II polypeptide as described herein, including for example, a polypeptide sequence comprising, consisting essentially of, or consisting of an amino acid sequence that is at least 80%, 85%, 90%, 95%, 96%, 97%, 98%, 99%, or 100% identical to the sequence of any one of SEQ ID NOs: 14, 15, 124, 126, 171, 172, 413, 414, 463, 464, 18, 19, 136, 138, 173, 174, 421, 422, 465, 466, 22, 23, 115, 117, 175, 176, 407, 408, 467, 468, 26, 27, 83, 84, 104, 106, 177, 178, 403, 404, 469, 470, 30, 31, 87, 88, 139, 141, 179, 180, 423, 424, 471, 472, 34, 35, 91, 92, 142, 144, 181, 182, 425, 426, 473, 474, 38, 39, 301, 302, 305, 306, 309, 310, 313, 112, 114, 183, 184, 405, 406, 475, 476, 9, 10, 11, 118, 120, 151, 152, 409, 410, 451, 452, 1, 2, 3, 4, 5, 6, 100, 102, 153, 154, 401, 402, 453, 454, 46, 47, 71, 72, 121, 123, 155, 156, 411, 412, 455, 456, 50, 51, 75, 76, 79, 80, 133, 135, 161, 162, 419, 420, 457, 458, 42, 43, 67, 68, 127, 129, 130, 132, 157, 158, 159, 160, 415, 416, 417, 418, 459, 460, 461, and 462. An interaction pair may be selected to confer an improved property/activity such as increased serum half-life, or to act as an adaptor on to which another moiety is attached to provide an improved property/activity. For example, a polyethylene glycol moiety may be attached to one or both components of an interaction pair to provide an improved property/activity such as improved serum half-life.

The first and second members of the interaction pair may be an asymmetric pair, meaning that the members of the pair preferentially associate with each other rather than self-associate. Accordingly, first and second members of an asymmetric interaction pair may associate to form a heterodimeric complex (see, e.g., FIG. 2). Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

As specific examples, the present disclosure provides fusion proteins comprising TGF-beta superfamily type I or type II polypeptides fused to a polypeptide comprising a constant domain of an immunoglobulin, such as a CH1, CH2, or CH3 domain of an immunoglobulin or an Fc domain. Fc domains derived from human IgG1, IgG2, IgG3, and IgG4 are provided herein. Other mutations are known that decrease either CDC or ADCC activity, and collectively, any of these variants are included in the disclosure and may be used as advantageous components of a heteromultimeric complex of the disclosure. Optionally, the IgG1 Fc domain of SEQ ID NO: 208 has one or more mutations at residues such as Asp-265, Lys-322, and Asn-434 (numbered in accordance with the corresponding full-length IgG1). In certain cases, the mutant Fc domain having one or more of these mutations (e.g., Asp-265 mutation) has reduced ability of binding to the Fcγ receptor relative to a wildtype Fc domain. In other cases, the mutant Fc domain having one or more of these mutations (e.g., Asn-434 mutation) has increased ability of binding to the MHC class I-related Fc-receptor (FcRN) relative to a wildtype Fc domain.

An example of a native amino acid sequence that may be used for the Fc portion of human IgG1 (G1Fc) is shown below (SEQ ID NO: 208). Dotted underline indicates the hinge region, and solid underline indicates positions with naturally occurring variants. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 208. Naturally occurring variants in G1Fc would include E134D and M136L according to the numbering system used in SEQ ID NO: 208 (see Uniprot P01857).

| | (SEQ ID NO: 208) |
|---|---|
| 1 | 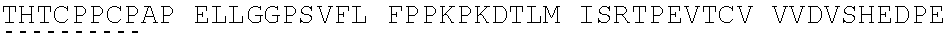 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

An example of a native amino acid sequence that may be used for the Fc portion of human IgG2 (G2Fc) is shown below (SEQ ID NO: 209). Dotted underline indicates the hinge region and double underline indicates positions where there are data base conflicts in the sequence (according to UniProt P01859). In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 209.

(SEQ ID NO: 209)

| | |
|---|---|
| 1 | 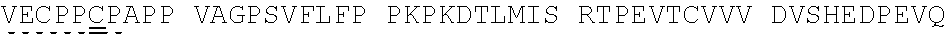 |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 210) contains a short hinge region consisting of a single 15-residue segment, whereas the second G3Fc sequence (SEQ ID NO: 211) contains a full-length hinge region. In each case, dotted underline indicates the hinge region, and solid underline indicates positions with naturally occurring variants according to UniProt P01859. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NOs: 210 and 211.

(SEQ ID NO: 210)

| | |
|---|---|
| 1 | 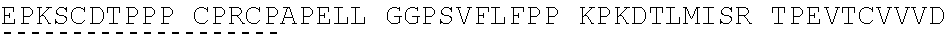 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |

(SEQ ID NO: 211)

| | |
|---|---|
| 1 | 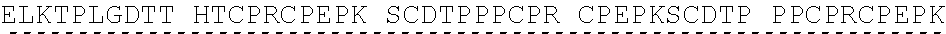 |
| 51 | 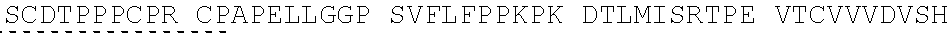 |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 210, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 212). Dotted underline indicates the hinge region. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 212.

(SEQ ID NO: 212)

| | |
|---|---|
| 1 | 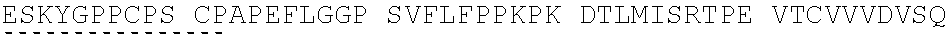 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

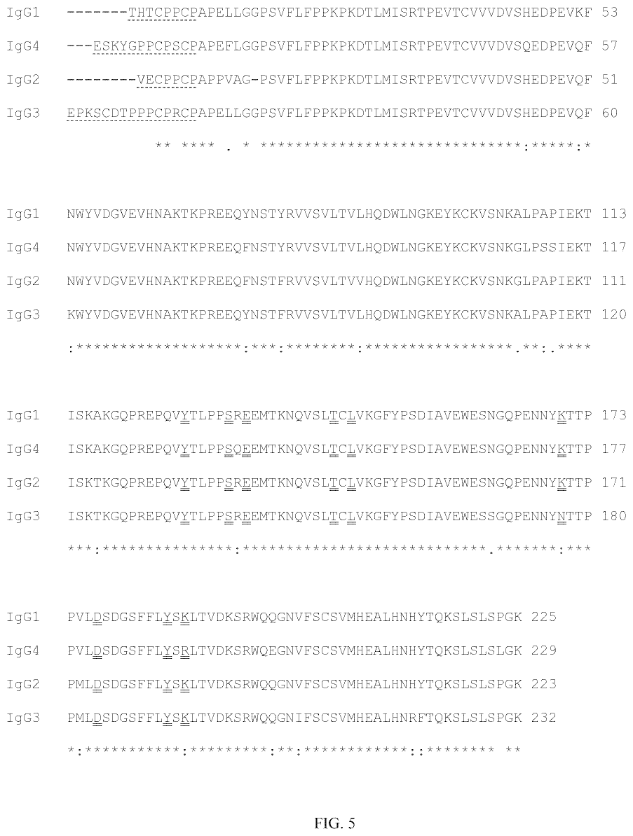

A variety of engineered mutations in the Fc domain are presented herein with respect to the G1Fc sequence (SEQ ID NO: 208), and analogous mutations in G2Fc, G3Fc, and G4Fc can be derived from their alignment with G1Fc in FIG. 5. Due to unequal hinge lengths, analogous Fc positions based on isotype alignment (FIG. 5) possess different amino acid numbers in SEQ ID NOs: 208, 209, 210, and 212. It can also be appreciated that a given amino acid position in an immunoglobulin sequence consisting of hinge, C_{H}2, and C_{H}3 regions (e.g., SEQ ID NOs: 208, 209, 210, 211, or 212) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 208), the human IgG1 heavy chain constant domain (Uniprot P01857), and the human IgG1 heavy chain is as follows.

Correspondence of C_{H}3 Positions in Different Numbering Systems

| G1Fc | IgG1 heavy chain | |
|---|---|---|
| (Numbering begins | constant domain | IgG1 heavy chain |
| at first threonine | (Numbering begins | (EU numbering scheme of |
| in hinge region) | at C_{H}1) | Kabat et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5^{th} ed., Vol. 1, NIH, Bethesda, MD.

A problem that arises in large-scale production of asymmetric immunoglobulin-based proteins from a single cell line is known as the “chain association issue”. As confronted prominently in the production of bispecific antibodies, the chain association issue concerns the challenge of efficiently producing a desired multichain protein from among the multiple combinations that inherently result when different heavy chains and/or light chains are produced in a single cell line [see, for example, Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

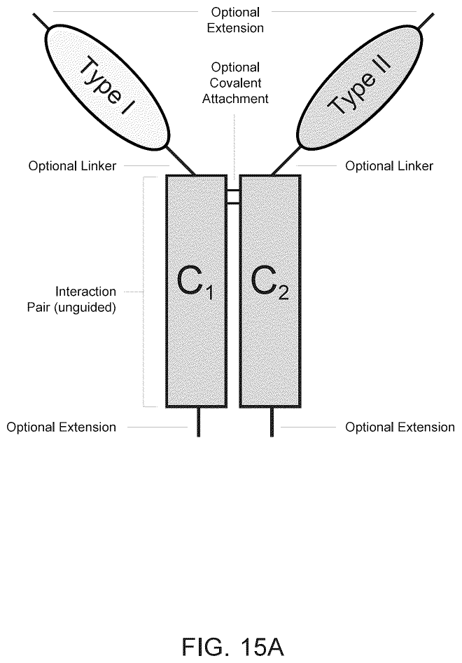

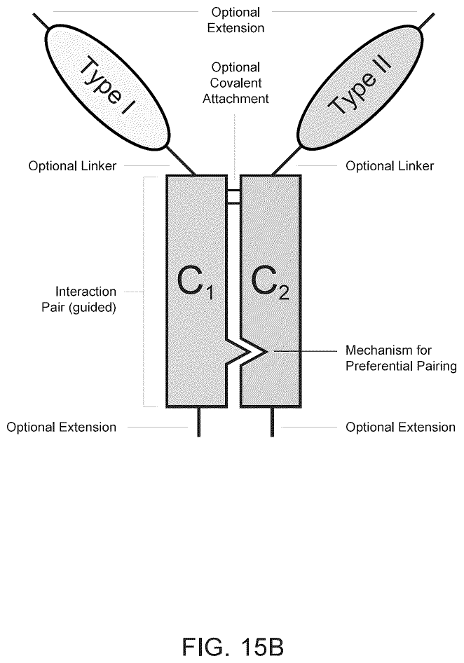

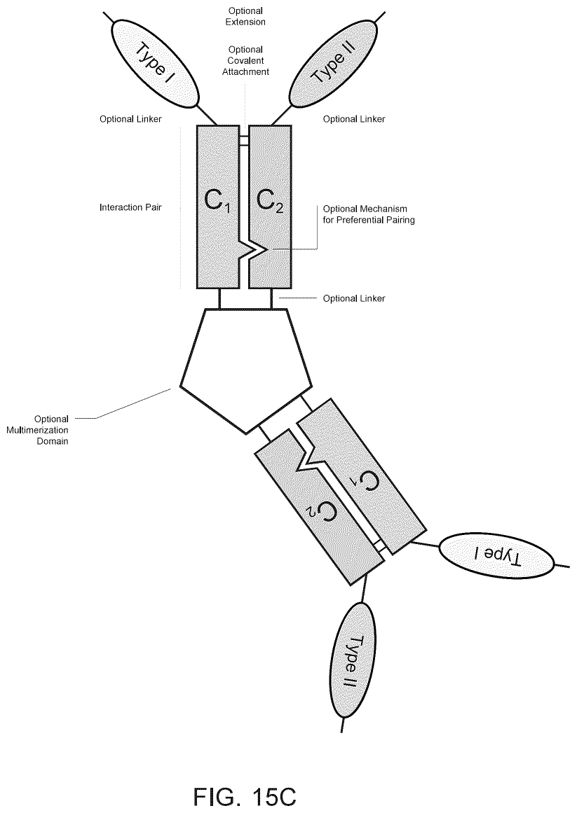

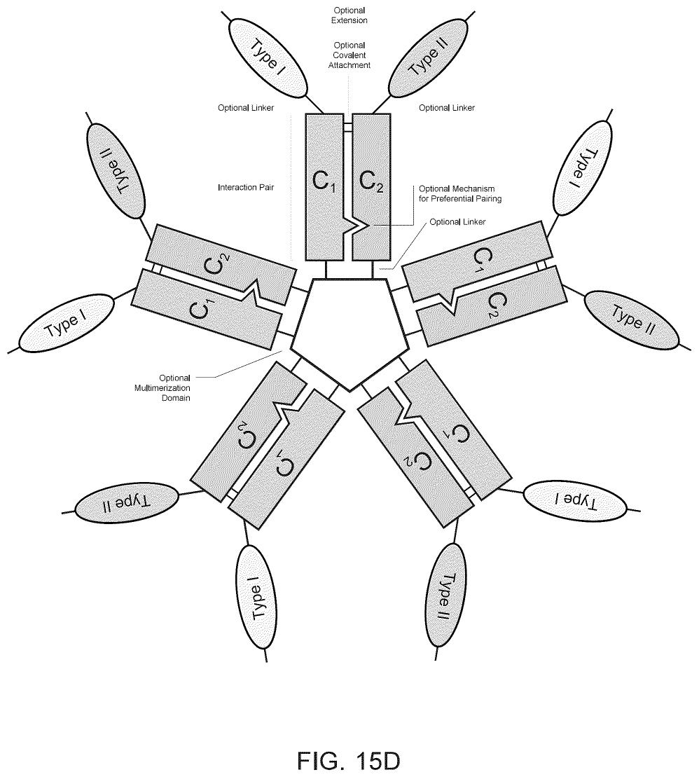

Various methods are known in the art that increase desired pairing of Fc-containing fusion polypeptide chains in a single cell line to produce a preferred asymmetric fusion protein at acceptable yields [see, for example, Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing. See, for example, Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605. As described herein, these methods may be used to generate heterodimers comprising TGF-beta superfamily type I and type II receptor polypeptides, at least two different TGF-beta superfamily type I receptor polypeptides, and at least two different TGF-beta superfamily type II receptor polypeptides. See FIGS. 15-17.

For example, one means by which interaction between specific polypeptides may be promoted is by engineering protuberance-into-cavity (knob-into-holes) complementary regions such as described in Arathoon et al., U.S. Pat. No. 7,183,076 and Carter et al., U.S. Pat. No. 5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged and lysine, arginine, and histidine are positively charged. These charged residues can be used to promote heterodimer formation and at the same time hinder homodimer formation. Attractive interactions take place between opposite charges and repulsive interactions occur between like charges. In part, protein complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (′)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399′ and D399-K409E′). A similar mutation switching the charge polarity (D399K′; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance heteromultimer formation of the polypeptide complexes disclosed herein.

Examples of Pair-Wise Charged Residue Mutations
to Enhance Heterodimer Formation

| | | | Corresponding |
|---|---|---|---|
| Position in | Mutation in | Interacting position | mutation in |
| first chain | first chain | in second chain | second chain |
| Lys409 | Asp or Glu | Asp399′ | Lys, Arg, or His |
| Lys392 | Asp or Glu | Asp399′ | Lys, Arg, or His |
| Lys439 | Asp or Glu | Asp356′ | Lys, Arg, or His |
| Lys370 | Asp or Glu | Glu357′ | Lys, Arg, or His |
| Asp399 | Lys, Arg, or His | Lys409′ | Asp or Glu |
| Asp399 | Lys, Arg, or His | Lys392′ | Asp or Glu |
| Asp356 | Lys, Arg, or His | Lys439′ | Asp or Glu |
| Glu357 | Lys, Arg, or His | Lys370′ | Asp or Glu |

In some embodiments, one or more residues that make up the CH3-CH3 interface in a fusion protein of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to the TGF-beta superfamily type I or type II receptor polypeptide of the construct, with or without an optional linker, to generate a TGF-beta superfamily type I or type II receptor fusion polypeptide This single chain can be coexpressed in a cell of choice along with the Fc sequence complementary to the first Fc to favor generation of the desired multichain construct (e.g., a TGF-beta superfamily heteromeric complex). In this example based on electrostatic steering, SEQ ID NO: 200 [human G1Fc(E134K/D177K)] and SEQ ID NO: 201 [human G1Fc(K170D/K187D)] are examples of complementary Fc sequences in which the engineered amino acid substitutions are double underlined, and the TGF-beta superfamily type I or type II receptor polypeptide of the construct can be fused to either SEQ ID NO: 200 or SEQ ID NO: 201, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 5) will generate complementary Fc pairs which may be used instead of the complementary hG1Fc pair below (SEQ ID NOs: 200 and 201).

| (SEQ ID NO: 200) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSRKEMTKNQ VSLTCLVKGF |
| 151 | YPSDIAVEWE SNGQPENNYK TTPPVLKSDG SFFLYSKLTV DKSRWQQGNV |
| 201 | FSCSVMHEAL HNHYTQKSLS LSPGK |
| (SEQ ID NO: 201) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYD TTPPVLDSDG SFFLYSDLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to the TGF-beta superfamily type I or type II polypeptide of the construct, with or without an optional linker, to generate a TGF-beta superfamily type I or type II fusion polypeptide. This single chain can be coexpressed in a cell of choice along with the Fc sequence complementary to the first Fc to favor generation of the desired multichain construct. In this example based on knobs-into-holes pairing, SEQ ID NO: 202 [human G1Fc(T144Y)] and SEQ ID NO: 203 [human G1Fc(Y185T)] are examples of complementary Fc sequences in which the engineered amino acid substitutions are double underlined, and the TGF-beta superfamily type I or type II polypeptide of the construct can be fused to either SEQ ID NO: 202 or SEQ ID NO: 203, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 5) will generate complementary Fc pairs which may be used instead of the complementary hG1Fc pair below (SEQ ID NOs: 202 and 203).

| (SEQ ID NO: 202) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLYCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 203) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLTSKLTV DKSRWQQGNV |
| 201 | FSCSVMHEAL HNHYTQKSLS LSPGK |

An example of Fc complementarity based on knobs-into-holes pairing combined with an engineered disulfide bond is disclosed in SEQ ID NO: 204 [hG1Fc(S132C/T144W)] and SEQ ID NO: 205 [hG1Fc(Y127C/T144S/L146A/Y185V)]. The engineered amino acid substitutions in these sequences are double underlined, and the TGF-beta superfamily type I or type II polypeptide of the construct can be fused to either SEQ ID NO: 204 or SEQ ID NO: 205, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 5) will generate complementary Fc pairs which may be used instead of the complementary hG1Fc pair below (SEQ ID NOs: 204 and 205).

| (SEQ ID NO: 204) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 205) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SNGQPENNYK TTPPVLDSDG SFFLVSKLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion proteins [see, for example, Davis et al (2010) Protein Eng Design Sel 23:195-202]. One of a pair of Fc sequences with SEEDbody complementarity can be arbitrarily fused to the TGF-beta superfamily type I or type II polypeptide of the construct, with or without an optional linker, to generate a TGF-beta superfamily type I or type II fusion polypeptide. This single chain can be coexpressed in a cell of choice along with the Fc sequence complementary to the first Fc to favor generation of the desired multichain construct. In this example based on SEEDbody (Sb) pairing, SEQ ID NO: 206 [hG1Fe(Sb_{AG})] and SEQ ID NO: 207 [hG1Fe(Sb_{GA})] are examples of complementary IgG Fc sequences in which the engineered amino acid substitutions from IgA Fc are double underlined, and the TGF-beta superfamily type I or type II polypeptide of the construct can be fused to either SEQ ID NO: 206 or SEQ ID NO: 207, but not both. Given the high degree of amino acid sequence identity between native hG1Fc, native hG2Fc, native hG3Fc, and native hG4Fc, it can be appreciated that amino acid substitutions at corresponding positions in hG1Fe, hG2Fe, hG3Fe, or hG4Fc (see FIG. 5) will generate an Fc monomer which may be used in the complementary IgG-IgA pair below (SEQ ID NOs: 206 and 207).

| (SEQ ID NO: 206) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PFRPEVHLLP PSREEMTKNQ VSLTCLARGF |
| 151 | YPKDIAVEWE SNGQPENNYK TIPSRQEPSQ GTTTFAVTSK LTVDKSRWQQ |
| 201 | GNVFSCSVMH EALHNHYTQK TISLSPGK |
| (SEQ ID NO: 207) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PPSEELALNE LVTLTCLVKG |
| 151 | FYPSDIAVEW ESNGQELPRE KYLTWAPVLD SDGSFFLYSI LRVAAEDWKK |
| 201 | GDTFSCSVMH EALHNHYTQK SLDRSPGK |

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains. See, e.g., Wranik et al (2012) J Biol Chem 287:43331-43339. As disclosed herein, one of a pair of Fc sequences attached to a leucine zipper-forming strand can be arbitrarily fused to the TGF-beta superfamily type I or type II polypeptide of the construct, with or without an optional linker, to generate a TGF-beta superfamily type I or type II fusion polypeptide. This single chain can be coexpressed in a cell of choice along with the Fc sequence attached to a complementary leucine zipper-forming strand to favor generation of the desired multichain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 213 [hG1Fc-Ap1 (acidic)] and SEQ ID NO: 214 [hG1Fc-Bp1 (basic)] are examples of complementary IgG Fc sequences in which the engineered complimentary leucine zipper sequences are underlined, and the TGF-beta superfamily type I or type II polypeptide of the construct can be fused to either SEQ ID NO: 213 or SEQ ID NO: 214, but not both. Given the high degree of amino acid sequence identity between native hG1Fc, native hG2Fc, native hG3Fc, and native hG4Fc, it can be appreciated that leucine zipper-forming sequences attached, with or without an optional linker, to hG1Fe, hG2Fe, hG3Fe, or hG4Fc (see FIG. 5) will generate an Fc monomer which may be used in the complementary leucine zipper-forming pair below (SEQ ID NOs: 213 and 214).

| (SEQ ID NO: 213) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LEKELQALEK ENAQLEWELQ |
| 251 | ALEKELAQGA T |
| (SEQ ID NO: 214) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LKKKLQALKK KNAQLKWKLQ |
| 251 | ALKKKLAQGA T |

As described above, various methods are known in the art that increase desired pairing of Fc-containing fusion polypeptide chains in a single cell line to produce a preferred asymmetric fusion protein at acceptable yields [Klein et al (2012) mAbs 4:653-663; and Spiess et al (2015) Molecular Immunology 67(2A): 95-106]. In addition, heteromultimers as described herein may be generated using a combination of heavy and light chain fusion proteins comprising either an TGF-beta superfamily type I or TGF-beta superfamily type II polypeptide. For example, in some embodiments, a TGF-beta superfamily type I receptor polypeptide polypeptide may be fused, with or without a linker domain, to an immunoglobulin heavy chain (IgG1, IgG2, IgG3, IgG4, IgM, IgA1, or IgA2) that comprises at least a portion of the C_{H}1 domain. Similarly, a TGF-beta superfamily type II receptor polypeptide may be fused, with or without a linker domain, to an immunoglobulin light chain (kappa or lambda) that comprises at least a portion of the light chain constant domain (C_{L}). In alternative embodiments, a TGF-beta superfamily type I receptor polypeptide may be fused, with or without a linker domain, to an immunoglobulin heavy chain (IgG1, IgG2, IgG3, IgG4, IgM, IgA1, or IgA2) that comprises at least a portion of the C_{H}1 domain, and a TGF-beta superfamily type II receptor polypeptide may be fused, with or without a linker domain, to an immunoglobulin light chain (kappa or lambda) that comprises at least a portion of the light chain constant domain (C_{L}). This design takes advantage of the natural ability of the heavy chains to heterodimerize with light chains. In particular, heterodimerization of a heavy and light chain occurs between the C_{H}1 with the C_{L}, which is generally stabilized by covalent linking of the two domains via a disulfide bridge. Constructs employing the full-length heavy chain, or at least a portion of the heavy chain comprising the hinge region, could give rise to antibody-like molecules comprising two “light chains” and two “heavy chains”. See FIG. 16. A potential advantage of this design is that it may more closely mimic the naturally occurring TGF-beta superfamily type I receptor polypeptide-ligand-TGF-beta superfamily type II receptor polypeptide complex and may display higher affinity for the ligand than comparable single heterodimers. In some embodiments, this design may be modified by incorporating various heavy chain truncations including, for example, truncations that comprise the C_{H}1 domain and some or all of the hinge domain (giving rise to F(ab′)₂-like molecules) as well as truncations that only comprise the C_{H}1 domain or a fragment thereof (giving rise to Fab-like molecules). See FIG. 16G. Various methods for designing such heteromultimer constructs are described in US 2009/0010879, Klein et al [(2012) mAbs 4:653-663], and Spiess et al [(2015) Molecular Immunology 67(2A): 95-106] the contents of which are incorporated in their entirety herein.

In some embodiments, it is desirable to generate antibody-like heterodimers comprising at least one branch of the complex comprising an TGF-beta superfamily type I receptor polypeptide-C_{L}:TGF-beta superfamily type II receptor polypeptide-C_{H}1 heterodimer pair and at least a second branch comprising an TGF-beta superfamily type II receptor polypeptide-C_{L}:TGF-beta superfamily type I receptor polypeptide r-C_{H}1 heterodimer pair. See, e.g., FIG. 16B. Such heterodimer complexes can be generated, for example, using combinations of heavy chain and light chain asymmetrical pairing technologies [Spiess et al (2015) Molecular Immunology 67(2A): 95-106]. For example, in CrossMab technology, [Schaefer et al (2011). Proc. Natl. Acad. Sci. U.S.A. 108: 11187-11192] light chain mispairing is overcome using domain crossovers and heavy chains heterodimerized using knobs-into-holes [Merchant et al (1998) Nat. Biotechnol. 16: 677-681]. For the domain crossovers either the variable domains or the constant domains are swapped between light and heavy chains to create two asymmetric Fab arms that drive cognate light chain pairing while preserving the structural and functional integrity of the variable domain [Fenn et al (2013) PLoS ONE 8: e61953]. An alternative approach for overcoming light chain mispairing is designing heavy and light chains with orthogonal Fab interfaces [Lewis (2014) Nat. Biotechnol. 32: 191-198]. This has been accomplished by computational modeling [Das et al (2008) Annu. Rev. Biochem. 77: 363-382] in combination with X-ray crystallography to identify mutations at the V_{H}/V_{L} and C_{H}1/C_{L} interfaces. For the heterodimers generated using this methodology, it may be necessary to engineer mutations into both V_{H}/V_{L} and CH1/C_{L} interfaces to minimize heavy/light chain mispairing. The designed orthogonal Fab interface may be used in conjunction with a heavy chain heterodimerization strategy to facilitate efficient IgG production in a single host cell. Electrostatic steering may also be used to generate orthogonal Fab interfaces to facilitate the construction of such heterodimers. Peptide linkers may be used to ensure cognate pairing of light and heavy chains in a format known as “LUZ-Y” [Wranik et al (2012) J. Biol. Chem. 287: 43331-43339], wherein heavy chain heterodimerization is accomplished using leucine zippers which may be subsequently removed by proteolysis in vitro.

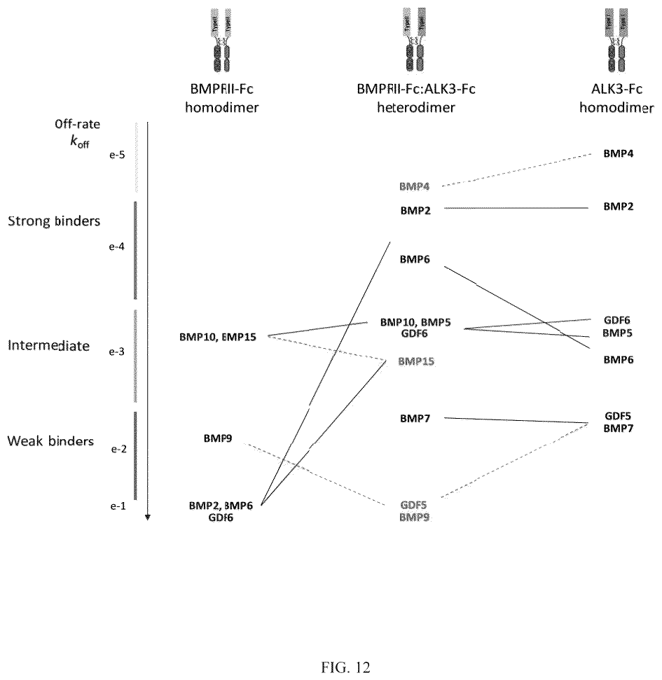

Alternatively, heteromultimers may comprise one or more single-chain ligand traps as described herein, optionally which may be covalently or non-covalently associated with one or more TGF-beta superfamily type I receptor polypeptides or TGF-beta superfamily type I receptor polypeptides as well as additional TGF-beta superfamily type I receptor polypeptide:TGF-beta superfamily type II receptor polypeptide single chain ligand traps [US 2011/0236309 and US2009/0010879]. See FIG. 12. As described herein, single-chain ligand traps do not require fusion to any multimerization domain such as coiled-coil Fc domains to be multivalent. In general, single-chain ligand traps of the present disclosure comprise at least one TGF-beta superfamily type I receptor polypeptide domain and one TGF-beta superfamily type I receptor polypeptide domain. The TGF-beta superfamily type I receptor polypeptide and TGF-beta superfamily type I receptor polypeptide domains, generally referred to herein as binding domains (BD), optionally may be joined by a linker region.

For example, in one aspect, the present disclosure provides heteromultimers comprising a polypeptide having the following structure:

(<BD1>-linker1)ₖ-[<BD2>-linker2-{<BD3>-linker3}_{f}]ₙ-(<BD4>)ₘ-(linker4-BD5>d)ₕ

where: n and h are independently greater than or equal to one; d, f, m, and k are independently equal to or greater than zero; BD1, BD2, BD3, BD4, and BD5 are independently TGF-beta superfamily type I receptor polypeptide or TGF-beta superfamily type II receptor polypeptide domains, wherein at least one of BD1, BD2, BD3, and BD4 is an TGF-beta superfamily type I receptor polypeptide domain, and wherein at least one of BD1, BD2, BD3, and BD4 is an TGF-beta superfamily type II receptor polypeptide domain, and linker1, linker2, linker3, and linker 4 are independently greater than or equal to zero. In some embodiment, TGF-beta superfamily type I receptor polypeptide:TGF-beta superfamily type II receptor polypeptide single-chain traps comprise at least two different TGF-beta superfamily type I receptor polypeptide. In some embodiments, TGF-beta superfamily type I receptor polypeptide:TGF-beta superfamily type II receptor polypeptide single-chain traps comprise at least two different TGF-beta superfamily type II receptor polypeptide polypeptides. In some embodiment, TGF-beta superfamily type I receptor polypeptide:TGF-beta superfamily type I receptor polypeptide single-chain traps comprise at least two different linkers. Depending on the values of selected for d, f, h, k, m, and n, the heteromultimer structure may comprise a large number of repeating units in various combinations or may be a relatively simple structure.

In another aspect, the present disclosure provides heteromultimers comprising a polypeptide having the following structure:

<BD1>-linker1-<BD2>

In yet another aspect, the present disclosure provides heteromultimers comprising a polypeptide having the following structure:

<BD1>-(linker2-<BD2>)ₙ

where n is greater than or equal one.

Another aspect of the invention provides heteromultimers comprising a polypeptide having the following structure:

(<BD1>-linker1-<BD1>)_{f}-linker2-(<BD2>-linker3-<BD3>)_{g}

wherein f and g are greater than or equal to one.

In an embodiment where BD2 and BD3 are the same, and f and g are the same number, this can result in a substantially mirror symmetric structure around linker 2, subject to differences in the linkers. In instances where BD2 is different from BD3 and/or where f and g are different numbers, different structures will be produced. It is within the capacity of one of ordinary skill in the art to select suitable binding domains, linkers, and repeat frequencies in light of the disclosure herein and knowledge in the art. Specific, non-limiting examples of such single-chain ligand traps in accordance with the present disclosure are represented schematically in FIG. 18.

The linkers (1, 2, 3, and 4) may be the same or different. The linker region provides a segment that is distinct from the structured ligand-binding domains of TGF-beta superfamily type I receptor polypeptide and TGF-beta superfamily type II receptor polypeptide and thus can be used for conjugation to accessory molecules (e.g., molecules useful in increasing stability such as PEGylation moieties) without having to chemically modify the binding domains. The linker may include an unstructured amino acid sequence that may be either the same as or derived from conservative modifications to the sequence of a natural unstructured region in the extracellular portion of the receptor for the ligand of interest or another receptor in the TGF-β superfamily. In other instances, such linkers may be entirely artificial in composition and origin but will contain amino acids selected to provide an unstructured flexible linker with a low likelihood of encountering electrostatic or steric hindrance complications when brought into close proximity to the ligand of interest. Linker length will be considered acceptable when it permits binding domains located on each of the N- and C-termini of the linker to bind their natural binding sites on their natural ligand such that, with both binding domains so bound, the ligand is bound with a higher affinity than it would be bound by binding of only one of the binding domains. In some instances, the number of amino acid residues in the linker of either natural or artificial origin is selected to be equal to or greater than the minimum required distance for simultaneous (bridged) binding to two binding sites on the TGF-beta superfamily type I receptor polypeptide and/or TGF-beta superfamily type II receptor polypeptide ligand. For example, and without wishing to be limiting in any manner, the linker length may be between about 1-10 amino acids, 10-20 amino acids, 18-80 amino acids, 25-60 amino acids, 35-45 amino acids, or any other suitable length.

Linkers may be designed to facilitate purification of the polypeptide. The exact purification scheme chosen will determine what modifications are needed, for example and without wishing to be limiting, additions of purification “tags” such as His tags is contemplated; in other examples, the linker may include regions to facilitate the addition of cargo or accessory molecules. When such additions affect the unstructured nature of the linker or introduce potential electrostatic or steric concerns, appropriate increases to the linker length will be made to ensure that the two binding domains are able to bind their respective sites on the ligand. In light of the methods and teachings herein, such determinations could be made routinely by one skilled in the art.

In addition, the present design permits linkage of other cargo molecules (for example imaging agents like fluorescent molecules), toxins, etc. For example, and without wishing to be limiting in any manner, single-chain polypeptides can be modified to add one or more cargo and/or accessory molecules (referred to collectively herein by R1, R2, R3, R4, etc.):

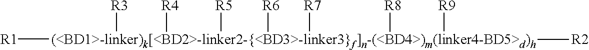

Without limiting the generality of R substituents available, R1, R2, R3, R4, R5, R6, R7, R8, R9, may or may not be present; when present, they may be the same or different, and may independently be one or more of: a fusion protein for targeting, for example, but not limited to such as an antibody fragment (e.g. single chain Fv) and/or a single domain antibody (sdAb); a radiotherapy and/or imaging agent, for example, but not limited to a radionuceotide (e.g. ¹²³I, ¹¹¹In, ¹⁸F, ⁶⁴C, ⁶⁸Y, ¹²⁴I, ¹³¹I, ⁹⁰Y, ¹⁷⁷Lu, ⁵⁷Cu, ²¹³Bi, ²¹¹At), a fluorescent dye (e.g. Alexa Fluor, Cy dye) and/or a fluorescent protein tag (e.g. GFP, DsRed); a cytotoxic agent for chemotherapy, for example, but not limited to doxorubicin, calicheamicin, a maytansinoid derivatives (e.g. DM1, DM4), a toxin (eg. truncated Pseudomonas endotoxin A, diphteria toxin); a nanoparticle-based carrier, for example, but not limited to polyethylene glycol (PEG), a polymer-conjugated to drug, nanocarrier or imaging agent (e.g. of a polymer N-(2-hydorxylpropyl) methacrylamide (HPMA), glutamic acid, PEG, dextran); a drug (for example, but not limited to doxorubicin, camptothecin, paclitaxel, palatinate); a nanocarrier, for example, but not limited to a nanoshell or liposome; an imaging agent, for example, but not limited to Supermagnetic Iron Oxide (SPIO); a dendrimer; and/or a solid support for use in ligand purification, concentration or sequestration (e.g. nanoparticles, inert resins, suitable silica supports).

In general, it will not be preferable to have cargo or accessory molecules in all possible positions, as this may cause steric or electrostatic complications. However, the effects of adding a cargo or accessory molecule to any given position or positions on the structure can be determined routinely in light of the disclosure herein by modeling the linker between the binding domains and carrying out molecular dynamics simulations to substantially minimize molecular mechanics energy and reduce steric and electrostatic incompatibility between the linker and the TGF-beta superfamily type I receptor polypeptide and TGF-beta superfamily type II receptor polypeptideas taught herein.

It may be preferable to add the cargo or accessory molecule to the linker portion of the agent, rather to the binding domain, to reduce the likelihood of interference in binding function. However, addition to the binding domain is possible and could be desirable in some instances and the effect of such an addition can be determined routinely in advance by modeling the binding agent and the linker with the proposed addition as described herein.

Conjugation methodologies may be performed using commercial kits that enable conjugation via common reactive groups such as primary amines, succinimidyl (NHS) esters and sulfhydral-reactive groups. Some non-limiting examples are: Alexa Fluor 488 protein labeling kit (Molecular Probes, Invitrogen detection technologies) and PEGylation kits (Pierce Biotechnology Inc.).

In certain aspects, TGF-beta superfamily type I receptor polypeptide:TGF-beta superfamily type II receptor polypeptide single-chain traps may be covalently or non-covalently associated with one or more TGF-beta superfamily type I receptor polypeptides or TGF-beta superfamily type II receptor polypeptide as well as additional TGF-beta superfamily type I receptor polypeptide:TGF-beta superfamily type II receptor polypeptide single chain ligand traps to form higher order heteromultimers, which may be used in accordance with the methods described herein. See, e.g., FIG. 19. For example, an TGF-beta superfamily type I receptor polypeptide: TGF-beta superfamily type II receptor polypeptide single chain ligand trap may further comprise a multimerization domain as described herein. In some embodiments, TGF-beta superfamily type I receptor polypeptide:TGF-beta superfamily type II receptor polypeptide single chain ligand traps comprise a constant domain of an Ig immunoglobulin. Such immunoglobulins constant domains may be selected to promote symmetrical or asymmetrical complexes comprising at least one single-chain TGF-beta superfamily type I receptor polypeptide:TGF-beta superfamily type II receptor polypeptide trap.

In certain aspects, an TGF-beta superfamily type I receptor polypeptide: TGF-beta superfamily type II receptor polypeptide single-chain trap, or combinations of such traps, may be used as TGF-beta superfamily antagonists to treat or prevent an TGF-beta superfamily disorder or disease as described herein (e.g., kidney disease and/or a metabolic disorder or condition).

It is understood that different elements of the fusion proteins (e.g., immunoglobulin Fc fusion proteins) may be arranged in any manner that is consistent with desired functionality. For example, a TGF-beta superfamily type I and/or type II receptor polypeptide domain may be placed C-terminal to a heterologous domain, or alternatively, a heterologous domain may be placed C-terminal to a TGF-beta superfamily type I and/or type II receptor polypeptide domain. The TGF-beta superfamily type I and/or type II receptor polypeptide domain and the heterologous domain need not be adjacent in a fusion protein, and additional domains or amino acid sequences may be included C- or N-terminal to either domain or between the domains.

For example, a TGF-beta superfamily type I and/or type II receptor fusion protein may comprise an amino acid sequence as set forth in the formula A-B-C. The B portion corresponds to a TGF-beta superfamily type I and/or type II receptor polypeptide domain. The A and C portions may be independently zero, one, or more than one amino acid, and both the A and C portions when present are heterologous to B. The A and/or C portions may be attached to the B portion via a linker sequence. A linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 58), GGGG (SEQ ID NO: 59), TGGGG (SEQ ID NO: 60), SGGGG (SEQ ID NO: 61), TGGG (SEQ ID NO: 62), or SGGG (SEQ ID NO: 63) singlets, or repeats. In certain embodiments, a TGF-beta superfamily type I and/or type II receptor fusion protein comprises an amino acid sequence as set forth in the formula A-B-C, wherein A is a leader (signal) sequence, B consists of a TGF-beta superfamily type I and/or type II receptor polypeptide domain, and C is a polypeptide portion that enhances one or more of in vivo stability, in vivo half-life, uptake/administration, tissue localization or distribution, formation of protein complexes, and/or purification. In certain embodiments, a TGF-beta superfamily type I and/or type II receptor fusion protein comprises an amino acid sequence as set forth in the formula A-B-C, wherein A is a TPA leader sequence, B consists of a TGF-beta superfamily type I and/or type II receptor polypeptide domain, and C is an immunoglobulin Fc domain. Preferred fusion proteins comprise the amino acid sequence set forth in any one of SEQ ID NOs: 100, 102, 104, 106, 112, 114, 115, 117, 118, 120, 121, 123, 124, 126, 127, 129, 401, 402, 403, 404, 405, 406, 407, 408, 409, 410, 411, 412, 413, 414, 415, and 416.

In some embodiments, TGF-beta superfamily receptor heteromultimer complexes of the present disclosure further comprise one or more heterologous portions (domains) so as to confer a desired property. For example, some fusion domains are particularly useful for isolation of the fusion proteins by affinity chromatography. Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆ (SEQ ID NO: 509)) fusion partners. As another example, a fusion domain may be selected so as to facilitate detection of the ligand trap polypeptides. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for factor Xa or thrombin, which allows the relevant protease to partially digest the fusion proteins and thereby liberate the recombinant proteins therefrom. The liberated proteins can then be isolated from the fusion domain by subsequent chromatographic separation.

In certain embodiments, TGF-beta superfamily type I and/or type II receptor polypeptides of the present disclosure contain one or more modifications that are capable of stabilizing the polypeptides. For example, such modifications enhance the in vitro half-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising a TGF-beta superfamily type I and/or type II receptor polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol.

In preferred embodiments, TGF-beta superfamily heteromultimer complexes to be used in accordance with the methods described herein are isolated polypeptide complexes. As used herein, an isolated protein (or protein complex) or polypeptide (or polypeptide complex) is one which has been separated from a component of its natural environment. In some embodiments, a heteromultimer complex of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of antibody purity are well known in the art [See, e.g., Flatman et al., (2007) J. Chromatogr. B 848:79-87]. In some embodiments, heteromultimer preparations of the disclosure are substantially free of TGF-beta superfamily type I receptor polypeptide homomultimers and/or TGF-beta superfamily type II receptor polypeptide homomultimers. For example, in some embodiments, heteromultimer preparations comprise less than about 10%, 9%, 8%, 7%, 5%, 4%, 3%, 2%, or less than 1% of TGF-beta superfamily type I receptor polypeptide homomultimers. In some embodiments, heteromultimer preparations comprise less than about 10%, 9%, 8%, 7%, 5%, 4%, 3%, 2%, or less than 1% of TGF-beta superfamily type II receptor polypeptide homomultimers. In some embodiments, heteromultimer preparations comprise less than about 10%, 9%, 8%, 7%, 5%, 4%, 3%, 2%, or less than 1% of TGF-beta superfamily type I receptor polypeptide homomultimers and less than about 10%, 9%, 8%, 7%, 5%, 4%, 3%, 2%, or less than 1% of TGF-beta superfamily type II receptor polypeptide homomultimers.

In certain embodiments, TGFβ superfamily type I and/or type II receptor polypeptides, as well as heteromultimer complexes thereof, of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (see, e.g., Advanced ChemTech Model 396; Milligen/Biosearch 9600). Alternatively, the polypeptides and complexes of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length TGFβ superfamily type I and/or type II receptor polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites.

With respect to antibodies that bind to and antagonize ligands that bind to TGF-beta type I receptor polypeptide:TGF-beta type II receptor polypeptide heteromultimers of the disclosure (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty) it is contemplated that an antibody may be designed as a bispecific antibody comprising a first portion that binds to an epitope of such ligand, such that the first portion of the antibody competes for binding with a type I receptor and comprising a second portion that binds to an epitope of such ligand, such that the second portion of the antibody competes for binding with a type II receptor. In this manner, a bispecific antibody targeting a single ligand can be designed to mimic the dual type I-type II receptor binding blockade that may be conferred by an ALK7:ActRIIB heteromultimer. Similarly it is contemplated that the same effect could be achieved using a combination of two or more antibodies wherein at least a first antibody binds to an epitope of such ligand, such that the first antibody competes for binding with a type I receptor and at least a second antibody binds to an epitope of such ligand, such that the second antibody competes for binding with a type II receptor.

B. Nucleic Acids Encoding TGFβ Superfamily Type I and/or Type II Receptor Polypeptides

In certain embodiments, the present disclosure provides isolated and/or recombinant nucleic acids encoding TGFβ superfamily type I and/or type II receptors (including fragments, functional variants, and fusion proteins thereof) disclosed herein. For example, SEQ ID NO: 12 encodes the naturally occurring human ActRIIA precursor polypeptide, while SEQ ID NO: 13 encodes the mature, extracellular domain of ActRIIA. The subject nucleic acids may be single-stranded or double stranded. Such nucleic acids may be DNA or RNA molecules. These nucleic acids may be used, for example, in methods for making TGF-beta superfamily heteromultimer complexes of the present disclosure.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding TGFβ superfamily type I and/or type II receptor polypeptides of the present disclosure are understood to include nucleic acids that are variants of any one of SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 101, 105, 113, 116, 119, 122, 125, 128, 131, 134, 137, 140, and 143. Variant nucleotide sequences include sequences that differ by one or more nucleotide substitutions, additions, or deletions including allelic variants, and therefore, will include coding sequences that differ from the nucleotide sequence designated in any one of SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 101, 105, 113, 116, 119, 122, 125, 128, 131, 134, 137, 140, and 143.

In certain embodiments, TGFβ superfamily type I and/or type II receptor polypeptides of the present disclosure are encoded by isolated or recombinant nucleic acid sequences that are at least 80%, 85%, 90%, 95%, 96%, 97%, 98%, 99%, or 100% identical to SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 101, 105, 113, 116, 119, 122, 125, 128, 131, 134, 137, 140, and 143. One of ordinary skill in the art will appreciate that nucleic acid sequences that are at least 80%, 85%, 90%, 95%, 96%, 97%, 98%, or 99% identical to the sequences complementary to SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 101, 105, 113, 116, 119, 122, 125, 128, 131, 134, 137, 140, and 143 are also within the scope of the present disclosure. In further embodiments, the nucleic acid sequences of the disclosure can be isolated, recombinant, and/or fused with a heterologous nucleotide sequence or in a DNA library.

In other embodiments, nucleic acids of the present disclosure also include nucleotide sequences that hybridize under highly stringent conditions to the nucleotide sequence designated in SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 101, 105, 113, 116, 119, 122, 125, 128, 131, 134, 137, 140, and 143, the complement sequence of SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 101, 105, 113, 116, 119, 122, 125, 128, 131, 134, 137, 140, and 143, or fragments thereof. One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 101, 105, 113, 116, 119, 122, 125, 128, 131, 134, 137, 140, and 143 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

In certain aspects of the present disclosure, the subject nucleic acid is provided in an expression vector comprising a nucleotide sequence encoding a TGFβ superfamily type I and/or type II receptor polypeptide and operably linked to at least one regulatory sequence. Regulatory sequences are art-recognized and are selected to direct expression of the TGFβ superfamily type I and/or type II receptor polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding a TGFβ superfamily type I and/or type II receptor polypeptides. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant TGFβ superfamily type I and/or type II receptor polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In a preferred embodiment, a vector will be designed for production of the subject TGFβ superfamily type I and/or type II receptor polypeptides in CHO cells, such as a Pcmv-Script vector (Stratagene, La Jolla, Calif.), pcDNA4 vectors (Invitrogen, Carlsbad, Calif) and pCI-neo vectors (Promega, Madison, Wisc.). As will be apparent, the subject gene constructs can be used to cause expression of the subject TGFβ superfamily type I and/or type II receptor polypeptide in cells propagated in culture, e.g., to produce proteins, including fusion proteins or variant proteins, for purification.

This disclosure also pertains to a host cell transfected with a recombinant gene including a coding sequence for one or more of the subject TGFβ superfamily type I and/or type II receptor polypeptides. The host cell may be any prokaryotic or eukaryotic cell. For example, a TGFβ superfamily type I and/or type II receptor polypeptide of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject TGFβ superfamily type I and/or type II receptor polypeptides. For example, a host cell transfected with an expression vector encoding a TGFβ superfamily type I and/or type II receptor polypeptide can be cultured under appropriate conditions to allow expression of the TGFβ superfamily type I and/or type II receptor polypeptide to occur. The polypeptide may be secreted and isolated from a mixture of cells and medium containing the polypeptide. Alternatively, the TGFβ superfamily type I and/or type II receptor polypeptide may be isolated from a cytoplasmic or membrane fraction obtained from harvested and lysed cells. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the TGFβ superfamily type I and/or type II receptor polypeptides and affinity purification with an agent that binds to a domain fused to TGFβ superfamily type I and/or type II receptor polypeptide (e.g., a protein A column may be used to purify a TGFβ superfamily type I receptor-Fc and/or type II receptor-Fc fusion protein). In some embodiments, the TGFβ superfamily type I and/or type II receptor polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. A TGFβ superfamily type I receptor-Fc and/or type II receptor-Fc fusion protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant TGFβ superfamily type I and/or type II receptor polypeptide, can allow purification of the expressed fusion protein by affinity chromatography using a Ni²⁺ metal resin. The purification leader sequence can then be subsequently removed by treatment with enterokinase to provide the purified TGFβ superfamily type I and/or type II receptor polypeptide. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 4. Screening Assays

In certain aspects, the present disclosure relates to the use of TGFβ superfamily type I and type II receptor heteromultimer complexes to identify compounds (agents) which are agonists or antagonists of TGFβ superfamily receptors. Compounds identified through this screening can be tested to assess their ability to modulate tissues such as bone, cartilage, muscle, fat, and/or neurons, to assess their ability to modulate tissue growth in vivo or in vitro. These compounds can be tested, for example, in animal models.

There are numerous approaches to screening for therapeutic agents for modulating tissue growth by targeting TGFβ superfamily ligand signaling (e.g., SMAD 2/3 and/or SMAD 1/5/8 signaling). In certain embodiments, high-throughput screening of compounds can be carried out to identify agents that perturb TGFβ superfamily receptor-mediated effects on a selected cell line. In certain embodiments, the assay is carried out to screen and identify compounds that specifically inhibit or reduce binding of a TGF-beta superfamily heteromultimer complex to its binding partner, such as a TGFβ superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). Alternatively, the assay can be used to identify compounds that enhance binding of a TGF-beta superfamily heteromultimer complex to its binding partner such as a TGFβ superfamily ligand. In a further embodiment, the compounds can be identified by their ability to interact with a TGF-beta superfamily heteromultimer complex of the disclosure.

A variety of assay formats will suffice and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatible crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a TGF-beta superfamily heteromultimer complex and its binding partner (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty).

Merely to illustrate, in an exemplary screening assay of the present disclosure, the compound of interest is contacted with an isolated and purified TGF-beta superfamily heteromultimer complex which is ordinarily capable of binding to a TGF-beta superfamily ligand, as appropriate for the intention of the assay. To the mixture of the compound and TGF-beta superfamily heteromultimer complex is then added to a composition containing the appropriate TGF-beta superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). Detection and quantification of heteromultimer-superfamily ligand complexes provides a means for determining the compound's efficacy at inhibiting (or potentiating) complex formation between the TGF-beta superfamily heteromultimer complex and its binding protein. The efficacy of the compound can be assessed by generating dose-response curves from data obtained using various concentrations of the test compound. Moreover, a control assay can also be performed to provide a baseline for comparison. For example, in a control assay, isolated and purified TGF-beta superfamily ligand is added to a composition containing the TGF-beta superfamily heteromultimer complex, and the formation of heteromultimer-ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Binding of a TGF-beta superfamily heteromultimer complex to another protein may be detected by a variety of techniques. For instance, modulation of the formation of complexes can be quantitated using, for example, detectably labeled proteins such as radiolabeled (e.g., ³²P ³⁵S ⁴C or ³H), fluorescently labeled (e.g., FITC), or enzymatically labeled TGF-beta superfamily heteromultimer complex and/or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a TGF-beta superfamily heteromultimer complex and its binding protein. Further, other modes of detection, such as those based on optical waveguides (see, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between a TGF-beta superfamily heteromultimer complex and its binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., small molecules or peptides) that dissociate interactions between a TGF-beta superfamily heteromultimer complex and its binding protein [see, e.g., Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with a TGF-beta superfamily heteromultimer complex of the disclosure. The interaction between the compound and the TGF-beta superfamily heteromultimer complex may be covalent or non-covalent. For example, such interaction can be identified at the protein level using in vitro biochemical methods, including photo-crosslinking, radiolabeled ligand binding, and affinity chromatography. See, e.g., Jakoby W B et al. (1974) Methods in Enzymology 46:1. In certain cases, the compounds may be screened in a mechanism-based assay, such as an assay to detect compounds which bind to a TGF-beta superfamily heteromultimer complex. This may include a solid-phase or fluid-phase binding event. Alternatively, the gene encoding a TGF-beta superfamily heteromultimer complex can be transfected with a reporter system (e.g., 0-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 5. Exemplary Therapeutic Uses

In certain embodiments, a TGF-beta superfamily heteromultimer complex, or combinations of TGF-beta superfamily heteromultimer complexes, of the present disclosure can be used to treat or prevent a disease or condition that is associated with abnormal activity of a TGFβ superfamily receptor (e.g., ALK1, ALK2, ALK3, ALK4, ALK5, ALK6, ALK7, ActRIIA, ActRIIB, BMPRII, TGFBRII, and MISRII) and/or a TGFβ superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). These diseases, disorders or conditions are generally referred to herein as “TGFβ superfamily-associated conditions” or “TGFβ superfamily-associated disorders.” In certain embodiments, the present invention provides methods of treating or preventing an individual in need thereof through administering to the individual a therapeutically effective amount of a TGF-beta superfamily heteromultimer complex, or combinations of TGF-beta superfamily heteromultimer complexes, as described herein. Any of the TGF-beta superfamily heteromultimer complexes of the present disclosure can potentially be employed individually or in combination for therapeutic uses disclosed herein. These methods are particularly aimed at therapeutic and prophylactic treatments of mammals including, for example, rodents, primates, and humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. The term “treating” as used herein includes amelioration or elimination of the condition once it has been established. In either case, prevention or treatment may be discerned in the diagnosis provided by a physician or other health care provider and the intended result of administration of the therapeutic agent.

TGFβ superfamily receptor-ligand complexes play essential roles in tissue growth as well as early developmental processes such as the correct formation of various structures or in one or more post-developmental capacities including sexual development, pituitary hormone production, and creation of bone and cartilage. Thus, TGFβ superfamily-associated conditions/disorders include abnormal tissue growth and developmental defects. In addition, TGFβ superfamily-associated conditions include, but are not limited to, disorders of cell growth and differentiation such as inflammation, allergy, autoimmune diseases, infectious diseases, and tumors.

Exemplary TGFβ superfamily-associated conditions include neuromuscular disorders (e.g., muscular dystrophy and muscle atrophy), congestive obstructive pulmonary disease (and muscle wasting associated with COPD), muscle wasting syndrome, sarcopenia, cachexia, adipose tissue disorders (e.g., obesity), type 2 diabetes (NIDDM, adult-onset diabetes), and bone degenerative disease (e.g., osteoporosis). Other exemplary TGFβ superfamily-associated conditions include musculodegenerative and neuromuscular disorders, tissue repair (e.g., wound healing), neurodegenerative diseases (e.g., amyotrophic lateral sclerosis), and immunologic disorders (e.g., disorders related to abnormal proliferation or function of lymphocytes).

In certain embodiments, a TGF-beta superfamily heteromultimer complex, or combinations of TGF-beta superfamily heteromultimer complexes, of the disclosure are used as part of a treatment for a muscular dystrophy. The term “muscular dystrophy” refers to a group of degenerative muscle diseases characterized by gradual weakening and deterioration of skeletal muscles and sometimes the heart and respiratory muscles. Muscular dystrophies are genetic disorders characterized by progressive muscle wasting and weakness that begin with microscopic changes in the muscle. As muscles degenerate over time, the person's muscle strength declines. Exemplary muscular dystrophies that can be treated with a regimen including the subject TGF-beta superfamily heteromultimer complexes include: Duchenne muscular dystrophy (DMD), Becker muscular dystrophy (BMD), Emery-Dreifuss muscular dystrophy (EDMD), limb-girdle muscular dystrophy (LGMD), facioscapulohumeral muscular dystrophy (FSH or FSHD) (also known as Landouzy-Dejerine), myotonic dystrophy (MMD; also known as Steinert's Disease), oculopharyngeal muscular dystrophy (OPMD), distal muscular dystrophy (DD), congenital muscular dystrophy (CMD).

Duchenne muscular dystrophy (DMD) was first described by the French neurologist Guillaume Benjamin Amand Duchenne in the 1860s. Becker muscular dystrophy (BMD) is named after the German doctor Peter Emil Becker, who first described this variant of DMD in the 1950s. DMD is one of the most frequent inherited diseases in males, affecting one in 3,500 boys. DMD occurs when the dystrophin gene, located on the short arm of the X chromosome, is defective. Since males only carry one copy of the X chromosome, they only have one copy of the dystrophin gene. Without the dystrophin protein, muscle is easily damaged during cycles of contraction and relaxation. While early in the disease muscle compensates by regeneration, later on muscle progenitor cells cannot keep up with the ongoing damage and healthy muscle is replaced by non-functional fibro-fatty tissue.

BMD results from different mutations in the dystrophin gene. BMD patients have some dystrophin, but it is either of insufficient quantity or poor quality. The presence of some dystrophin protects the muscles of patients with BMD from degenerating as severely or as quickly as those of patients with DMD.

Studies in animals indicate that inhibition of the GDF8 signaling pathway may effectively treat various aspects of disease in DMD and BMD patients (Bogdanovich et al., 2002, Nature 420:418-421; Pistilli et al., 2011, Am J Pathol 178:1287-1297). Thus, TGF-beta superfamily heteromultimer complexes of the disclosure may act as GDF8 inhibitors (antagonists), and constitute an alternative means of blocking signaling by GDF8 and/or related TGFβ superfamily ligands in vivo in DMD and BMD patients.

Similarly, TGF-beta superfamily heteromultimer complexes of the disclosure may provide an effective means to increase muscle mass in other disease conditions that are in need of muscle growth. For example, amyotrophic lateral sclerosis (ALS), also called Lou Gehrig's disease or motor neuron disease, is a chronic, progressive, and incurable CNS disorder that attacks motor neurons, which are components of the central nervous system required for initiation of skeletal muscle contraction. In ALS, motor neurons deteriorate and eventually die, and though a person's brain normally remains fully functioning and alert, initiation of muscle contraction is blocked at the spinal level. Individuals who develop ALS are typically between 40 and 70 years old, and the first motor neurons to degenerate are those innervating the arms or legs. Patients with ALS may have trouble walking, may drop things, fall, slur their speech, and laugh or cry uncontrollably. As the disease progresses, muscles in the limbs begin to atrophy from disuse. Muscle weakness becomes debilitating, and patients eventually require a wheel chair or become confined to bed. Most ALS patients die from respiratory failure or from complications of ventilator assistance like pneumonia 3-5 years from disease onset.

Promotion of increased muscle mass by TGF-beta superfamily heteromultimer complexes might also benefit those suffering from muscle wasting diseases. Gonzalez-Cadavid et al. (supra) reported that GDF8 expression correlates inversely with fat-free mass in humans and that increased expression of the GDF8 gene is associated with weight loss in men with AIDS wasting syndrome. By inhibiting the function of GDF8 in AIDS patients, at least certain symptoms of AIDS may be alleviated, if not completely eliminated, thus significantly improving quality of life in AIDS patients.

Since loss of GDF8 function is also associated with fat loss without diminution of nutrient intake (Zimmers et al., supra; McPherron and Lee, supra), the subject TGF-beta superfamily heteromultimer complexes may further be used as a therapeutic agent for slowing or preventing the development of obesity and type 2 diabetes.

Cancer anorexia-cachexia syndrome is among the most debilitating and life-threatening aspects of cancer. This syndrome is a common feature of many types of cancer—present in approximately 80% of cancer patients at death—and is responsible not only for a poor quality of life and poor response to chemotherapy but also a shorter survival time than is found in patients with comparable tumors but without weight loss. Cachexia is typically suspected in patients with cancer if an involuntary weight loss of greater than five percent of premorbid weight occurs within a six-month period. Associated with anorexia, wasting of fat and muscle tissue, and psychological distress, cachexia arises from a complex interaction between the cancer and the host. Cancer cachexia affects cytokine production, release of lipid-mobilizing and proteolysis-inducing factors, and alterations in intermediary metabolism. Although anorexia is common, a decreased food intake alone is unable to account for the changes in body composition seen in cancer patients, and increasing nutrient intake is unable to reverse the wasting syndrome. Currently, there is no treatment to control or reverse the cachexic process. Since systemic overexpression of GDF8 in adult mice was found to induce profound muscle and fat loss analogous to that seen in human cachexia syndromes (Zimmers et al., supra), the subject TGF-beta superfamily heteromultimer complex pharmaceutical compositions may be beneficially used to prevent, treat, or alleviate the symptoms of the cachexia syndrome, where muscle growth is desired. An example of a heteromeric complex useful for preventing, treating, or alleviating muscle loss as described above is ActRIIB-Fc:ALK4-Fc.

In certain embodiments, a TGF-beta superfamily heteromultimer complex, or combinations of TGF-beta superfamily heteromultimer complexes, of the present disclosure may be used in methods of inducing bone and/or cartilage formation, preventing bone loss, increasing bone mineralization, preventing the demineralization of bone, and/or increasing bone density. TGF-beta superfamily heteromultimer complexes may be useful in patients who are diagnosed with subclinical low bone density, as a protective measure against the development of osteoporosis.

In some embodiments, a TGF-beta superfamily heteromultimer complex, or combinations of TGF-beta superfamily heteromultimer complexes, of the present disclosure may find medical utility in the healing of bone fractures and cartilage defects in humans and other animals. The subject methods and compositions may also have prophylactic use in closed as well as open fracture reduction and also in the improved fixation of artificial joints. De novo bone formation induced by an osteogenic agent is useful for repair of craniofacial defects that are congenital, trauma-induced, or caused by oncologic resection, and is also useful in cosmetic plastic surgery. Further, methods and compositions of the invention may be used in the treatment of periodontal disease and in other tooth repair processes. In certain cases, a TGF-beta superfamily heteromultimer complex, or combinations of TGF-beta superfamily heteromultimer complexes, may provide an environment to attract bone-forming cells, stimulate growth of bone-forming cells, or induce differentiation of progenitors of bone-forming cells. TGF-beta superfamily heteromultimer complexes of the disclosure may also be useful in the treatment of osteoporosis. Further, TGF-beta superfamily heteromultimer complexes may be used in repair of cartilage defects and prevention/reversal of osteoarthritis. Examples of heteromeric complexes useful for inducing bone formation, preventing bone loss, increasing bone mineralization, preventing the demineralization of bone, and/or increasing bone density as described above are ActRIIB-Fc:ALK3-Fc and ActRIIB-Fc:ALK4-Fc.

Rosen et al. (ed) Primer on the Metabolic Bone Diseases and Disorders of Mineral Metabolism, 7^{th} ed. American Society for Bone and Mineral Research, Washington D.C. (incorporated herein by reference) provides an extensive discussion of bone disorders that may be subject to treatment with a TGF-beta superfamily heteromultimer complex or with combinations of TGF-beta superfamily heteromultimer complexes. A partial listing is provided herein. Methods and compositions of the invention can be applied to conditions characterized by or causing bone loss, such as osteoporosis (including secondary osteoporosis), hyperparathyroidism, chronic kidney disease mineral bone disorder, sex hormone deprivation or ablation (e.g. androgen and/or estrogen), glucocorticoid treatment, rheumatoid arthritis, severe burns, hyperparathyroidism, hypercalcemia, hypocalcemia, hypophosphatemia, osteomalacia (including tumor-induced osteomalacia), hyperphosphatemia, vitamin D deficiency, hyperparathyroidism (including familial hyperparathyroidism) and pseudohypoparathyroidism, tumor metastases to bone, bone loss as a consequence of a tumor or chemotherapy, tumors of the bone and bone marrow (e.g., multiple myeloma), ischemic bone disorders, periodontal disease and oral bone loss, Cushing's disease, Paget's disease, thyrotoxicosis, chronic diarrheal state or malabsorption, renal tubular acidosis, or anorexia nervosa. Methods and compositions of the invention may also be applied to conditions characterized by a failure of bone formation or healing, including non-union fractures, fractures that are otherwise slow to heal, fetal and neonatal bone dysplasias (e.g., hypocalcemia, hypercalcemia, calcium receptor defects and vitamin D deficiency), osteonecrosis (including osteonecrosis of the jaw) and osteogenesis imperfecta. Additionally, the anabolic effects will cause such antagonists to diminish bone pain associated with bone damage or erosion. As a consequence of the anti-resorptive effects, such antagonists may be useful to treat disorders of abnormal bone formation, such as osteoblastic tumor metastases (e.g., associated with primary prostate or breast cancer), osteogenic osteosarcoma, osteopetrosis, progressive diaphyseal dysplasia, endosteal hyperostosis, osteopoikilosis, and melorheostosis. Other disorders that may be treated include fibrous dysplasia and chondrodysplasias.

In another specific embodiment, the disclosure provides a therapeutic method and composition for repairing fractures and other conditions related to cartilage and/or bone defects or periodontal diseases. The invention further provides therapeutic methods and compositions for wound healing and tissue repair. The types of wounds include, but are not limited to, burns, incisions and ulcers. See, e.g., PCT Publication No. WO 84/01106. Such compositions comprise a therapeutically effective amount of at least one of the TGF-beta superfamily heteromultimer complexes of the disclosure in admixture with a pharmaceutically acceptable vehicle, carrier, or matrix.

In some embodiments, a TGF-beta superfamily heteromultimer complex, or combinations of TGF-beta superfamily heteromultimer complexes, of the disclosure can be applied to conditions causing bone loss such as osteoporosis, hyperparathyroidism, Cushing's disease, thyrotoxicosis, chronic diarrheal state or malabsorption, renal tubular acidosis, or anorexia nervosa. It is commonly appreciated that being female, having a low body weight, and leading a sedentary lifestyle are risk factors for osteoporosis (loss of bone mineral density, leading to fracture risk). However, osteoporosis can also result from the long-term use of certain medications. Osteoporosis resulting from drugs or another medical condition is known as secondary osteoporosis. In Cushing's disease, the excess amount of cortisol produceds by the body results in osteoporosis and fractures. The most common medications associated with secondary osteoporosis are the corticosteroids, a class of drugs that act like cortisol, a hormone produced naturally by the adrenal glands. Although adequate levels of thyroid hormones are needed for the development of the skeleton, excess thyroid hormone can decrease bone mass over time. Antacids that contain aluminum can lead to bone loss when taken in high doses by people with kidney problems, particularly those undergoing dialysis. Other medications that can cause secondary osteoporosis include phenytoin (Dilantin) and barbiturates that are used to prevent seizures; methotrexate (Rheumatrex, Immunex, Folex PFS), a drug for some forms of arthritis, cancer, and immune disorders; cyclosporine (Sandimmune, Neoral), a drug used to treat some autoimmune diseases and to suppress the immune system in organ transplant patients; luteinizing hormone-releasing hormone agonists (Lupron, Zoladex), used to treat prostate cancer and endometriosis; heparin (Calciparine, Liquaemin), an anticlotting medication; and cholestyramine (Questran) and colestipol (Colestid), used to treat high cholesterol. Bone loss resulting from cancer therapy is widely recognized and termed cancer therapy-induced bone loss (CTIBL). Bone metastases can create cavities in the bone that may be corrected by treatment with a TGF-beta superfamily heteromultimer complex. Bone loss can also be caused by gum disease, a chronic infection in which bacteria located in gum recesses produce toxins and harmful enzymes.

In a further embodiment, the present disclosure provides methods and therapeutic agents for treating diseases or disorders associated with abnormal or unwanted bone growth. For example, patients with the congenital disorder fibrodysplasia ossificans progressiva (FOP) are afflicted by progressive ectopic bone growth in soft tissues spontaneously or in response to tissue trauma, with a major impact on quality of life. Additionally, abnormal bone growth can occur after hip replacement surgery and thus ruin the surgical outcome. This is a more common example of pathological bone growth and a situation in which the subject methods and compositions may be therapeutically useful. The same methods and compositions may also be useful for treating other forms of abnormal bone growth (e.g., pathological growth of bone following trauma, burns or spinal cord injury), and for treating or preventing the undesirable conditions associated with the abnormal bone growth seen in connection with metastatic prostate cancer or osteosarcoma.

In certain embodiments, a TGF-beta superfamily heteromultimer complex, or combinations of TGF-beta superfamily heteromultimer complexes, of the disclosure may be used to promote bone formation in patients with cancer. Patients having certain tumors (e.g. prostate, breast, multiple myeloma or any tumor causing hyperparathyroidism) are at high risk for bone loss due to tumor-induced bone loss, bone metastases, and therapeutic agents. Such patients may be treated with a TGF-beta superfamily heteromultimer complex, or a combination of complexes, even in the absence of evidence of bone loss or bone metastases. Patients may also be monitored for evidence of bone loss or bone metastases, and may be treated with a TGF-beta superfamily heteromultimer complex in the event that indicators suggest an increased risk. Generally, DEXA scans are employed to assess changes in bone density, while indicators of bone remodeling may be used to assess the likelihood of bone metastases. Serum markers may be monitored. Bone specific alkaline phosphatase (BSAP) is an enzyme that is present in osteoblasts. Blood levels of BSAP are increased in patients with bone metastasis and other conditions that result in increased bone remodeling. Osteocalcin and procollagen peptides are also associated with bone formation and bone metastases. Increases in BSAP have been detected in patients with bone metastasis caused by prostate cancer, and to a lesser degree, in bone metastases from breast cancer. BMP7 levels are high in prostate cancer that has metastasized to bone, but not in bone metastases due to bladder, skin, liver, or lung cancer. Type I carboxy-terminal telopeptide (ICTP) is a crosslink found in collagen that is formed during to the resorption of bone. Since bone is constantly being broken down and reformed, ICTP will be found throughout the body. However, at the site of bone metastasis, the level will be significantly higher than in an area of normal bone. ICTP has been found in high levels in bone metastasis due to prostate, lung, and breast cancer. Another collagen crosslink, Type I N-terminal telopeptide (NTx), is produced along with ICTP during bone turnover. The amount of NTx is increased in bone metastasis caused by many different types of cancer including lung, prostate, and breast cancer. Also, the levels of NTx increase with the progression of the bone metastasis. Therefore, this marker can be used to both detect metastasis as well as measure the extent of the disease. Other markers of resorption include pyridinoline and deoxypyridinoline. Any increase in resorption markers or markers of bone metastases indicate the need for therapy with a TGF-beta superfamily heteromultimer complex, or combinations of TGF-beta superfamily heteromultimer complexes, in a patient.

A TGF-beta superfamily heteromultimer complex, or combinations of TGF-beta superfamily heteromultimer complexes, of the disclosure may be conjointly administered with other bone-active pharmaceutical agents. Conjoint administration may be accomplished by administration of a single co-formulation, by simultaneous administration, or by administration at separate times. TGF-beta superfamily heteromultimer complexes may be particularly advantageous if administered with other bone-active agents. A patient may benefit from conjointly receiving a TGF-beta superfamily heteromultimer complex and taking calcium supplements, vitamin D, appropriate exercise and/or, in some cases, other medication. Examples of other medications incude, bisphosphonates (alendronate, ibandronate and risedronate), calcitonin, estrogens, parathyroid hormone and raloxifene. The bisphosphonates (alendronate, ibandronate and risedronate), calcitonin, estrogens and raloxifene affect the bone remodeling cycle and are classified as anti-resorptive medications. Bone remodeling consists of two distinct stages: bone resorption and bone formation. Anti-resorptive medications slow or stop the bone-resorbing portion of the bone-remodeling cycle but do not slow the bone-forming portion of the cycle. As a result, new formation continues at a greater rate than bone resorption, and bone density may increase over time. Teriparatide, a form of parathyroid hormone, increases the rate of bone formation in the bone remodeling cycle. Alendronate is approved for both the prevention (5 mg per day or 35 mg once a week) and treatment (10 mg per day or 70 mg once a week) of postmenopausal osteoporosis. Alendronate reduces bone loss, increases bone density and reduces the risk of spine, wrist and hip fractures. Alendronate also is approved for treatment of glucocorticoid-induced osteoporosis in men and women as a result of long-term use of these medications (i.e., prednisone and cortisone) and for the treatment of osteoporosis in men. Alendronate plus vitamin D is approved for the treatment of osteoporosis in postmenopausal women (70 mg once a week plus vitamin D), and for treatment to improve bone mass in men with osteoporosis. Ibandronate is approved for the prevention and treatment of postmenopausal osteoporosis. Taken as a once-a-month pill (150 mg), ibandronate should be taken on the same day each month. Ibandronate reduces bone loss, increases bone density and reduces the risk of spine fractures. Risedronate is approved for the prevention and treatment of postmenopausal osteoporosis. Taken daily (5 mg dose) or weekly (35 mg dose or 35 mg dose with calcium), risedronate slows bone loss, increases bone density and reduces the risk of spine and non-spine fractures. Risedronate also is approved for use by men and women to prevent and/or treat glucocorticoid-induced osteoporosis that results from long-term use of these medications (i.e., prednisone or cortisone). Calcitonin is a naturally occurring hormone involved in calcium regulation and bone metabolism. In women who are more than 5 years beyond menopause, calcitonin slows bone loss, increases spinal bone density, and may relieve the pain associated with bone fractures. Calcitonin reduces the risk of spinal fractures. Calcitonin is available as an injection (50-100 IU daily) or nasal spray (200 IU daily).

A patient may also benefit from conjointly receiving a TGF-beta superfamily heteromultimer complex, or combinations of TGF-beta superfamily heteromultimer complexes, and additional bone-active medications. Estrogen therapy (ET)/hormone therapy (HT) is approved for the prevention of osteoporosis. ET has been shown to reduce bone loss, increase bone density in both the spine and hip, and reduce the risk of hip and spinal fractures in postmenopausal women. ET is administered most commonly in the form of a pill or skin patch that delivers a low dose of approximately 0.3 mg daily or a standard dose of approximately 0.625 mg daily and is effective even when started after age 70. When estrogen is taken alone, it can increase a woman's risk of developing cancer of the uterine lining (endometrial cancer). To eliminate this risk, healthcare providers prescribe the hormone progestin in combination with estrogen (hormone replacement therapy or HT) for those women who have an intact uterus. ET/HT relieves menopause symptoms and has been shown to have a beneficial effect on bone health. Side effects may include vaginal bleeding, breast tenderness, mood disturbances and gallbladder disease. Raloxifene, 60 mg a day, is approved for the prevention and treatment of postmenopausal osteoporosis. It is from a class of drugs called Selective Estrogen Receptor Modulators (SERMs) that have been developed to provide the beneficial effects of estrogens without their potential disadvantages. Raloxifene increases bone mass and reduces the risk of spine fractures. Data are not yet available to demonstrate that raloxifene can reduce the risk of hip and other non-spine fractures. Teriparatide, a form of parathyroid hormone, is approved for the treatment of osteoporosis in postmenopausal women and men who are at high risk for a fracture. This medication stimulates new bone formation and significantly increases bone mineral density. In postmenopausal women, fracture reduction was noted in the spine, hip, foot, ribs and wrist. In men, fracture reduction was noted in the spine, but there were insufficient data to evaluate fracture reduction at other sites. Teriparatide is self-administered as a daily injection for up to 24 months.

In other embodiments, a TGF-beta superfamily heteromultimer complex, or combinations of TGF-beta superfamily heteromultimer complexes can be used for regulating body fat content in an animal and for treating or preventing conditions related thereto, and particularly, health-compromising conditions related thereto. According to the present invention, to regulate (control) body weight can refer to reducing or increasing body weight, reducing or increasing the rate of weight gain, or increasing or reducing the rate of weight loss, and also includes actively maintaining, or not significantly changing body weight (e.g., against external or internal influences which may otherwise increase or decrease body weight). One embodiment of the present disclosure relates to regulating body weight by administering to an animal (e.g., a human) in need thereof a TGF-beta superfamily heteromultimer complex, or combinations of TGF-beta superfamily heteromultimer complexese, of the disclosure.

In some embodiments, a TGF-beta superfamily heteromultimer complex, or combinations of TGF-beta superfamily heteromultimer complexes, of the present disclosure can be used for reducing body weight and/or reducing weight gain in an animal, and more particularly, for treating or ameliorating obesity in patients at risk for or suffering from obesity. In another specific embodiment, the present invention is directed to methods and compounds for treating an animal that is unable to gain or retain weight (e.g., an animal with a wasting syndrome). Such methods are effective to increase body weight and/or mass, or to reduce weight and/or mass loss, or to improve conditions associated with or caused by undesirably low (e.g., unhealthy) body weight and/or mass. In addition, disorders of high cholesterol (e.g., hypercholesterolemia or dislipidemia) may be treated with a TGF-beta superfamily heteromultimer complex, or combinations of TGF-beta superfamily heteromultimer complexes, of the disclosure.

In certain aspects, a TGF-beta superfamily heteromultimer complex, or a combination of TGF-beta superfamily heteromultimer complexes, of the present disclosure can be used to increase red blood cell levels, treat or prevent an anemia, and/or treat or prevent ineffective erythropoiesis in a subject in need thereof. In certain aspects, a TGF-beta superfamily heteromultimer complex, or a combination of TGF-beta superfamily heteromultimer complexes, of the present disclosure may be used in combination with conventional therapeutic approaches for increasing red blood cell levels, particularly those used to treat anemias of multifactorial origin. Conventional therapeutic approaches for increasing red blood cell levels include, for example, red blood cell transfusion, administration of one or more EPO receptor activators, hematopoietic stem cell transplantation, immunosuppressive biologics and drugs (e.g., corticosteroids). In certain embodiments, a TGF-beta superfamily heteromultimer complex, or a combination of TGF-beta superfamily heteromultimer complexes, of the present disclosure can be used to treat or prevent ineffective erythropoiesis and/or the disorders associated with ineffective erythropoiesis in a subject in need thereof. In certain aspects, a TGF-beta superfamily heteromultimer complex, or a combination of TGF-beta superfamily heteromultimer complexes, of the present disclosure can be used in combination with conventional therapeutic approaches for treating or preventing an anemia or ineffective erythropoiesis disorder, particularly those used to treat anemias of multifactorial origin.

In general, treatment or prevention of a disease or condition as described in the present disclosure is achieved by administering a TGF-beta superfamily heteromultimer complex, or a combination of TGF-beta superfamily heteromultimer complexes, of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain embodiments, a TGF-beta superfamily heteromultimer complex, or a combination of TGF-beta superfamily heteromultimer complexes, optionally combined with an EPO receptor activator, may be used to increase red blood cell, hemoglobin, or reticulocyte levels in healthy individuals and selected patient populations. Examples of appropriate patient populations include those with undesirably low red blood cell or hemoglobin levels, such as patients having an anemia, and those that are at risk for developing undesirably low red blood cell or hemoglobin levels, such as those patients who are about to undergo major surgery or other procedures that may result in substantial blood loss. In one embodiment, a patient with adequate red blood cell levels is treated with a TGF-beta superfamily heteromultimer complex, or a combination of TGF-beta superfamily heteromultimer complexes, to increase red blood cell levels, and then blood is drawn and stored for later use in transfusions.

One or more TGF-beta superfamily heteromultimer complexes of the disclosure, optionally combined with an EPO receptor activator, may be used to increase red blood cell levels, hemoglobin levels, and/or hematocrit levels in a patient having an anemia. When observing hemoglobin and/or hematocrit levels in humans, a level of less than normal for the appropriate age and gender category may be indicative of anemia, although individual variations are taken into account. For example, a hemoglobin level from 10-12.5 g/dl, and typically about 11.0 g/dl is considered to be within the normal range in health adults, although, in terms of therapy, a lower target level may cause fewer cardiovascular side effects [see, e.g., Jacobs et al. (2000) Nephrol Dial Transplant 15, 15-19]. Alternatively, hematocrit levels (percentage of the volume of a blood sample occupied by the cells) can be used as a measure for anemia. Hematocrit levels for healthy individuals range from about 41-51% for adult males and from 35-45% for adult females. In certain embodiments, a patient may be treated with a dosing regimen intended to restore the patient to a target level of red blood cells, hemoglobin, and/or hematocrit. As hemoglobin and hematocrit levels vary from person to person, optimally, the target hemoglobin and/or hematocrit level can be individualized for each patient.

Anemia is frequently observed in patients having a tissue injury, an infection, and/or a chronic disease, particularly cancer. In some subjects, anemia is distinguished by low erythropoietin levels and/or an inadequate response to erythropoietin in the bone marrow [see, e.g., Adamson (2008) Harrison's Principles of Internal Medicine, 17th ed.; McGraw Hill, New York, pp 628-634]. Potential causes of anemia include, for example, blood loss, nutritional deficits (e.g. reduced dietary intake of protein), medication reaction, various problems associated with the bone marrow, and many diseases. More particularly, anemia has been associated with a variety of disorders and conditions that include, for example, bone marrow transplantation; solid tumors (e.g., breast cancer, lung cancer, and colon cancer); tumors of the lymphatic system (e.g., chronic lymphocyte leukemia, non-Hodgkins lymphoma, and Hodgkins lymphoma); tumors of the hematopoietic system (e.g., leukemia, a myelodysplastic syndrome and multiple myeloma); radiation therapy; chemotherapy (e.g., platinum containing regimens); inflammatory and autoimmune diseases, including, but not limited to, rheumatoid arthritis, other inflammatory arthritides, systemic lupus erythematosis (SLE), acute or chronic skin diseases (e.g., psoriasis), inflammatory bowel disease (e.g., Crohn's disease and ulcerative colitis); acute or chronic renal disease or failure, including idiopathic or congenital conditions; acute or chronic liver disease; acute or chronic bleeding; situations where transfusion of red blood cells is not possible due to patient allo- or auto-antibodies and/or for religious reasons (e.g., some Jehovah's Witnesses); infections (e.g., malaria and osteomyelitis); hemoglobinopathies including, for example, sickle cell disease (anemia), thalassemias; drug use or abuse (e.g., alcohol misuse); pediatric patients with anemia from any cause to avoid transfusion; and elderly patients or patients with underlying cardiopulmonary disease with anemia who cannot receive transfusions due to concerns about circulatory overload [see, e.g., Adamson (2008) Harrison's Principles of Internal Medicine, 17th ed.; McGraw Hill, New York, pp 628-634]. In some embodiments, one or more TGF-beta superfamily heteromultimer complexes of the disclosure could be used to treat or prevent anemia associated with one or more of the disorders or conditions disclosed herein.

Many factors can contribute to cancer-related anemia. Some are associated with the disease process itself and the generation of inflammatory cytokines such as interleukin-1, interferon-gamma, and tumor necrosis factor [Bron et al. (2001) Semin Oncol 28(Suppl 8):1-6]. Among its effects, inflammation induces the key iron-regulatory peptide hepcidin, thereby inhibiting iron export from macrophages and generally limiting iron availability for erythropoiesis [see, e.g., Ganz (2007) J Am Soc Nephrol 18:394-400]. Blood loss through various routes can also contribute to cancer-related anemia. The prevalence of anemia due to cancer progression varies with cancer type, ranging from 5% in prostate cancer up to 90% in multiple myeloma. Cancer-related anemia has profound consequences for patients, including fatigue and reduced quality of life, reduced treatment efficacy, and increased mortality. In some embodiments, one or more TGF-beta superfamily heteromultimer complexes of the disclosure, optionally combined with an EPO receptor activator, could be used to treat a cancer-related anemia.

A hypoproliferative anemia can result from primary dysfunction or failure of the bone marrow. Hypoproliferative anemias include: anemia of chronic disease, anemia of kidney disease, anemia associated with hypometabolic states, and anemia associated with cancer. In each of these types, endogenous erythropoietin levels are inappropriately low for the degree of anemia observed. Other hypoproliferative anemias include: early-stage iron-deficient anemia, and anemia caused by damage to the bone marrow. In these types, endogenous erythropoietin levels are appropriately elevated for the degree of anemia observed. Prominent examples would be myelosuppression caused by cancer and/or chemotherapeutic drugs or cancer radiation therapy. A broad review of clinical trials found that mild anemia can occur in 100% of patients after chemotherapy, while more severe anemia can occur in up to 80% of such patients [see, e.g., Groopman et al. (1999) J Natl Cancer Inst 91:1616-1634]. Myelosuppressive drugs include, for example: 1) alkylating agents such as nitrogen mustards (e.g., melphalan) and nitrosoureas (e.g., streptozocin); 2) antimetabolites such as folic acid antagonists (e.g., methotrexate), purine analogs (e.g., thioguanine), and pyrimidine analogs (e.g., gemcitabine); 3) cytotoxic antibiotics such as anthracyclines (e.g., doxorubicin); 4) kinase inhibitors (e.g., gefitinib); 5) mitotic inhibitors such as taxanes (e.g., paclitaxel) and vinca alkaloids (e.g., vinorelbine); 6) monoclonal antibodies (e.g., rituximab); and 7) topoisomerase inhibitors (e.g., topotecan and etoposide). In addition, conditions resulting in a hypometabolic rate can produce a mild-to-moderate hypoproliferative anemia. Among such conditions are endocrine deficiency states. For example, anemia can occur in Addison's disease, hypothyroidism, hyperparathyroidism, or males who are castrated or treated with estrogen. In some embodiments, one or more TGF-beta superfamily heteromultimer complexes of the disclosure, optionally combined with an EPO receptor activator, could be used to treat a hyperproliferative anemia.

Chronic kidney disease is sometimes associated with hypoproliferative anemia, and the degree of the anemia varies in severity with the level of renal impairment. Such anemia is primarily due to inadequate production of erythropoietin and reduced survival of red blood cells. Chronic kidney disease usually proceeds gradually over a period of years or decades to end-stage (Stage 5) disease, at which point dialysis or kidney transplantation is required for patient survival. Anemia often develops early in this process and worsens as disease progresses. The clinical consequences of anemia of kidney disease are well-documented and include development of left ventricular hypertrophy, impaired cognitive function, reduced quality of life, and altered immune function [see, e.g., Levin et al. (1999) Am J Kidney Dis 27:347-354; Nissenson (1992) Am J Kidney Dis 20(Suppl 1):21-24; Revicki et al. (1995) Am J Kidney Dis 25:548-554; Gafter et al., (1994) Kidney Int 45:224-231]. In some embodiments, one or more TGF-beta superfamily heteromultimer complexes of the disclosure, optionally combined with an EPO receptor activator, could be used to treat anemia associated with acute or chronic renal disease or failure.

Anemia resulting from acute blood loss of sufficient volume, such as from trauma or postpartum hemorrhage, is known as acute post-hemorrhagic anemia. Acute blood loss initially causes hypovolemia without anemia since there is proportional depletion of RBCs along with other blood constituents. However, hypovolemia will rapidly trigger physiologic mechanisms that shift fluid from the extravascular to the vascular compartment, which results in hemodilution and anemia. If chronic, blood loss gradually depletes body iron stores and eventually leads to iron deficiency. In some embodiments, one or more TGF-beta superfamily heteromultimer complexes of the disclosure, optionally combined with an EPO receptor activator, could be used to treat anemia resulting from acute blood loss.

Iron-deficiency anemia is the final stage in a graded progression of increasing iron deficiency which includes negative iron balance and iron-deficient erythropoiesis as intermediate stages. Iron deficiency can result from increased iron demand, decreased iron intake, or increased iron loss, as exemplified in conditions such as pregnancy, inadequate diet, intestinal malabsorption, acute or chronic inflammation, and acute or chronic blood loss. With mild-to-moderate anemia of this type, the bone marrow remains hypoproliferative, and RBC morphology is largely normal; however, even mild anemia can result in some microcytic hypochromic RBCs, and the transition to severe iron-deficient anemia is accompanied by hyperproliferation of the bone marrow and increasingly prevalent microcytic and hypochromic RBCs [see, e.g., Adamson (2008) Harrison's Principles of Internal Medicine, 17th ed.; McGraw Hill, New York, pp 628-634]. Appropriate therapy for iron-deficiency anemia depends on its cause and severity, with oral iron preparations, parenteral iron formulations, and RBC transfusion as major conventional options. In some embodiments, one or more TGF-beta superfamily heteromultimer complexes of the disclosure, optionally combined with an EPO receptor activator, could be used to treat a chronic iron-deficiency.

Myelodysplastic syndrome (MDS) is a diverse collection of hematological conditions characterized by ineffective production of myeloid blood cells and risk of transformation to acute myelogenous leukemia. In MDS patients, blood stem cells do not mature into healthy red blood cells, white blood cells, or platelets. MDS disorders include, for example, refractory anemia, refractory anemia with ringed sideroblasts, refractory anemia with excess blasts, refractory anemia with excess blasts in transformation, refractory cytopenia with multilineage dysplasia, and myelodysplastic syndrome associated with an isolated 5q chromosome abnormality. As these disorders manifest as irreversible defects in both quantity and quality of hematopoietic cells, most MDS patients are afflicted with chronic anemia. Therefore, MDS patients eventually require blood transfusions and/or treatment with growth factors (e.g., erythropoietin or G-CSF) to increase red blood cell levels. However, many MDS patients develop side-effects due to frequency of such therapies. For example, patients who receive frequent red blood cell transfusion can exhibit tissue and organ damage from the buildup of extra iron. Accordingly, one or more TGF-beta superfamily heteromultimer complexes of the disclosure, may be used to treat patients having MDS. In certain embodiments, patients suffering from MDS may be treated using one or more TGF-beta superfamily heteromultimer complexes of the disclosure, optionally in combination with an EPO receptor activator. In other embodiments, patients suffering from MDS may be treated using a combination of one or more TGF-beta superfamily heteromultimer complexes of the disclosure and one or more additional therapeutic agents for treating MDS including, for example, thalidomide, lenalidomide, azacitadine, decitabine, erythropoietins, deferoxamine, antithymocyte globulin, and filgrastrim (G-CSF).

Originally distinguished from aplastic anemia, hemorrhage, or peripheral hemolysis on the basis of ferrokinetic studies [see, e.g., Ricketts et al. (1978) Clin Nucl Med 3:159-164], ineffective erythropoiesis describes a diverse group of anemias in which production of mature RBCs is less than would be expected given the number of erythroid precursors (erythroblasts) present in the bone marrow [Tanno et al. (2010) Adv Hematol 2010:358283]. In such anemias, tissue hypoxia persists despite elevated erythropoietin levels due to ineffective production of mature RBCs. A vicious cycle eventually develops in which elevated erythropoietin levels drive massive expansion of erythroblasts, potentially leading to splenomegaly (spleen enlargement) due to extramedullary erythropoiesis [see, e.g., Aizawa et al. (2003) Am J Hematol 74:68-72], erythroblast-induced bone pathology [see, e.g., Di Matteo et al. (2008) J Biol Regul Homeost Agents 22:211-216], and tissue iron overload, even in the absence of therapeutic RBC transfusions [see, e.g., Pippard et al. (1979) Lancet 2:819-821]. Thus, by boosting erythropoietic effectiveness, one or more TGF-beta superfamily heteromultimer complexes of the present disclosure may break the aforementioned cycle and thus alleviate not only the underlying anemia but also the associated complications of elevated erythropoietin levels, splenomegaly, bone pathology, and tissue iron overload. In some embodiments, one or more TGF-beta superfamily heteromultimer complexes of the present disclosure can be used to treat or prevent ineffective erythropoiesis, including anemia and elevated EPO levels as well as complications such as splenomegaly, erythroblast-induced bone pathology, iron overload, and their attendant pathologies. With splenomegaly, such pathologies include thoracic or abdominal pain and reticuloendothelial hyperplasia. Extramedullary hematopoiesis can occur not only in the spleen but potentially in other tissues in the form of extramedullary hematopoietic pseudotumors [see, e.g., Musallam et al. (2012) Cold Spring Harb Perspect Med 2:a013482]. With erythroblast-induced bone pathology, attendant pathologies include low bone mineral density, osteoporosis, and bone pain [see, e.g., Haidar et al. (2011) Bone 48:425-432]. With iron overload, attendant pathologies include hepcidin suppression and hyperabsorption of dietary iron [see, e.g., Musallam et al. (2012) Blood Rev 26(Suppl 1):S16-S19], multiple endocrinopathies and liver fibrosis/cirrhosis [see, e.g., Galanello et al. (2010) Orphanet J Rare Dis 5:11], and iron-overload cardiomyopathy [Lekawanvijit et al., 2009, Can J Cardiol 25:213-218].

The most common causes of ineffective erythropoiesis are the thalassemia syndromes, hereditary hemoglobinopathies in which imbalances in the production of intact alpha- and beta-hemoglobin chains lead to increased apoptosis during erythroblast maturation [see, e.g., Schrier (2002) Curr Opin Hematol 9:123-126]. Thalassemias are collectively among the most frequent genetic disorders worldwide, with changing epidemiologic patterns predicted to contribute to a growing public health problem in both the U.S. and globally [Vichinsky (2005) Ann NY Acad Sci 1054:18-24]. Thalassemia syndromes are named according to their severity. Thus, α-thalassemias include α-thalassemia minor (also known as α-thalassemia trait; two affected α-globin genes), hemoglobin H disease (three affected α-globin genes), and α-thalassemia major (also known as hydrops fetalis; four affected α-globin genes). β-Thalassemias include β-thalassemia minor (also known as β-thalassemia trait; one affected β-globin gene), β-thalassemia intermedia (two affected β-globin genes), hemoglobin E thalassemia (two affected β-globin genes), and β-thalassemia major (also known as Cooley's anemia; two affected β-globin genes resulting in a complete absence of β-globin protein). β-Thalassemia impacts multiple organs, is associated with considerable morbidity and mortality, and currently requires life-long care. Although life expectancy in patients with β-thalassemia has increased in recent years due to use of regular blood transfusions in combination with iron chelation, iron overload resulting both from transfusions and from excessive gastrointestinal absorption of iron can cause serious complications such as heart disease, thrombosis, hypogonadism, hypothyroidism, diabetes, osteoporosis, and osteopenia [see, e.g., Rund et al. (2005) N Engl J Med 353:1135-1146]. In certain embodiments, one or more TGF-beta superfamily heteromultimer complexes of the disclosure, optionally combined with an EPO receptor activator, can be used to treat or prevent a thalassemia syndrome.

In some embodiments, one or more TGF-beta superfamily heteromultimer complexes of the disclosure, optionally combined with an EPO receptor activator, can be used for treating disorders of ineffective erythropoiesis besides thalassemia syndromes. Such disorders include siderblastic anemia (inherited or acquired); dyserythropoietic anemia (types I and II); sickle cell anemia; hereditary spherocytosis; pyruvate kinase deficiency; megaloblastic anemias, potentially caused by conditions such as folate deficiency (due to congenital diseases, decreased intake, or increased requirements), cobalamin deficiency (due to congenital diseases, pernicious anemia, impaired absorption, pancreatic insufficiency, or decreased intake), certain drugs, or unexplained causes (congenital dyserythropoietic anemia, refractory megaloblastic anemia, or erythroleukemia); myelophthisic anemias including, for example, myelofibrosis (myeloid metaplasia) and myelophthisis; congenital erythropoietic porphyria; and lead poisoning.

In certain embodiments, one or more TGF-beta superfamily heteromultimer complexes of the disclosure may be used in combination with supportive therapies for ineffective erythropoiesis. Such therapies include transfusion with either red blood cells or whole blood to treat anemia. In chronic or hereditary anemias, normal mechanisms for iron homeostasis are overwhelmed by repeated transfusions, eventually leading to toxic and potentially fatal accumulation of iron in vital tissues such as heart, liver, and endocrine glands. Thus, supportive therapies for patients chronically afflicted with ineffective erythropoiesis also include treatment with one or more iron-chelating molecules to promote iron excretion in the urine and/or stool and thereby prevent, or reverse, tissue iron overload [see, e.g., Hershko (2006) Haematologica 91:1307-1312; Cao et al. (2011), Pediatr Rep 3(2):e17]. Effective iron-chelating agents should be able to selectively bind and neutralize ferric iron, the oxidized form of non-transferrin bound iron which likely accounts for most iron toxicity through catalytic production of hydroxyl radicals and oxidation products [see, e.g., Esposito et al. (2003) Blood 102:2670-2677]. These agents are structurally diverse, but all possess oxygen or nitrogen donor atoms able to form neutralizing octahedral coordination complexes with individual iron atoms in stoichiometries of 1:1 (hexadentate agents), 2:1 (tridentate), or 3:1 (bidentate) [Kalinowski et al. (2005) Pharmacol Rev 57:547-583]. In general, effective iron-chelating agents also are relatively low molecular weight (e.g., less than 700 daltons), with solubility in both water and lipids to enable access to affected tissues. Specific examples of iron-chelating molecules include deferoxamine, a hexadentate agent of bacterial origin requiring daily parenteral administration, and the orally active synthetic agents deferiprone (bidentate) and deferasirox (tridentate). Combination therapy consisting of same-day administration of two iron-chelating agents shows promise in patients unresponsive to chelation monotherapy and also in overcoming issues of poor patient compliance with dereroxamine alone [Cao et al. (2011) Pediatr Rep 3(2):e17; Galanello et al. (2010) Ann NY Acad Sci 1202:79-86].

As used herein, “in combination with” or “conjoint administration” refers to any form of administration such that the second therapy is still effective in the body (e.g., the two compounds are simultaneously effective in the patient, which may include synergistic effects of the two compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, an individual who receives such treatment can benefit from a combined effect of different therapies. One or more TGF-beta superfamily heteromultimer complexes of the disclosure can be administered concurrently with, prior to, or subsequent to, one or more other additional agents or supportive therapies. In general, each therapeutic agent will be administered at a dose and/or on a time schedule determined for that particular agent. The particular combination to employ in a regimen will take into account compatibility of the antagonist of the present disclosure with the therapy and/or the desired therapeutic effect to be achieved.

In certain embodiments, one or more TGF-beta superfamily heteromultimer complexes of the disclosure may be used in combination with hepcidin or a hepcidin agonist for ineffective erythropoiesis. A circulating polypeptide produced mainly in the liver, hepcidin is considered a master regulator of iron metabolism by virtue of its ability to induce the degradation of ferroportin, an iron-export protein localized on absorptive enterocytes, hepatocytes, and macrophages. Broadly speaking, hepcidin reduces availability of extracellular iron, so hepcidin agonists may be beneficial in the treatment of ineffective erythropoiesis [see, e.g., Nemeth (2010) Adv Hematol 2010:750643]. This view is supported by beneficial effects of increased hepcidin expression in a mouse model of β-thalassemia [Gardenghi et al. (2010) J Clin Invest 120:4466-4477].

One or more TGF-beta superfamily heteromultimer complexes of the disclosure, optionally combined with an EPO receptor activator, would also be appropriate for treating anemias of disordered RBC maturation, which are characterized in part by undersized (microcytic), oversized (macrocytic), misshapen, or abnormally colored (hypochromic) RBCs.

In certain embodiments, the present disclosure provides methods of treating or preventing anemia in an individual in need thereof by administering to the individual a therapeutically effective amount of one or more TGF-beta superfamily heteromultimer complexes of the disclosure and a EPO receptor activator. In certain embodiments, one or more TGF-beta superfamily heteromultimer complexes of the disclosure may be used in combination with EPO receptor activators to reduce the required dose of these activators in patients that are susceptible to adverse effects of EPO. These methods may be used for therapeutic and prophylactic treatments of a patient.

One or more TGF-beta superfamily heteromultimer complexes of the disclosure may be used in combination with EPO receptor activators to achieve an increase in red blood cells, particularly at lower dose ranges of EPO receptor activators. This may be beneficial in reducing the known off-target effects and risks associated with high doses of EPO receptor activators. The primary adverse effects of EPO include, for example, an excessive increase in the hematocrit or hemoglobin levels and polycythemia. Elevated hematocrit levels can lead to hypertension (more particularly aggravation of hypertension) and vascular thrombosis. Other adverse effects of EPO which have been reported, some of which relate to hypertension, are headaches, influenza-like syndrome, obstruction of shunts, myocardial infarctions and cerebral convulsions due to thrombosis, hypertensive encephalopathy, and red cell blood cell aplasia. See, e.g., Singibarti (1994) J. Clin Investig 72(suppl 6), S36-S43; Horl et al. (2000) Nephrol Dial Transplant 15(suppl 4), 51-56; Delanty et al. (1997) Neurology 49, 686-689; and Bunn (2002) N Engl J Med 346(7), 522-523).

Provided that TGF-beta superfamily heteromultimer complexes of the present disclosure act by a different mechanism than EPO, these antagonists may be useful for increasing red blood cell and hemoglobin levels in patients that do not respond well to EPO. For example, a TGF-beta superfamily heteromultimer complex of the present disclosure may be beneficial for a patient in which administration of a normal-to-increased dose of EPO (>300 IU/kg/week) does not result in the increase of hemoglobin level up to the target level. Patients with an inadequate EPO response are found in all types of anemia, but higher numbers of non-responders have been observed particularly frequently in patients with cancers and patients with end-stage renal disease. An inadequate response to EPO can be either constitutive (observed upon the first treatment with EPO) or acquired (observed upon repeated treatment with EPO).

In certain embodiments, the present disclosure provides methods for managing a patient that has been treated with, or is a candidate to be treated with, one or more TGF-beta superfamily heteromultimer complexes of the disclosure by measuring one or more hematologic parameters in the patient. The hematologic parameters may be used to evaluate appropriate dosing for a patient who is a candidate to be treated with the antagonist of the present disclosure, to monitor the hematologic parameters during treatment, to evaluate whether to adjust the dosage during treatment with one or more antagonist of the disclosure, and/or to evaluate an appropriate maintenance dose of one or more antagonists of the disclosure. If one or more of the hematologic parameters are outside the normal level, dosing with one or more TGF-beta superfamily heteromultimer complexes of the disclosure may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art-recognized methods. Such parameters may be determined using a blood sample from a patient. Increases in red blood cell levels, hemoglobin levels, and/or hematocrit levels may cause increases in blood pressure.

In one embodiment, if one or more hematologic parameters are outside the normal range or on the high side of normal in a patient who is a candidate to be treated with one or more TGF-beta superfamily heteromultimer complexes of the disclosure, then onset of administration of the one or more TGF-beta superfamily heteromultimer complexes of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. For patients having higher than normal red blood cell levels and/or hemoglobin levels, then administration of the one or more TGF-beta superfamily heteromultimer complexes of the disclosure may be delayed until the levels have returned to a normal or acceptable level.

In certain embodiments, if one or more hematologic parameters are outside the normal range or on the high side of normal in a patient who is a candidate to be treated with one or more TGF-beta superfamily heteromultimer complexes of the disclosure, then the onset of administration may not be delayed. However, the dosage amount or frequency of dosing of the one or more TGF-beta superfamily heteromultimer complexes of the disclosure may be set at an amount that would reduce the risk of an unacceptable increase in the hematologic parameters arising upon administration of the one or more TGF-beta superfamily heteromultimer complexes of the disclosure. Alternatively, a therapeutic regimen may be developed for the patient that combines one or more TGF-beta superfamily heteromultimer complexes of the disclosure with a therapeutic agent that addresses the undesirable level of the hematologic parameter. For example, if the patient has elevated blood pressure, then a therapeutic regimen involving administration of one or more TGF-beta superfamily heteromultimer complexes of the disclosure and a blood pressure-lowering agent may be designed. For a patient having lower than desired iron stores, a therapeutic regimen of one or more TGF-beta superfamily heteromultimer complexes of the disclosure and iron supplementation may be developed.

In one embodiment, baseline parameter(s) for one or more hematologic parameters may be established for a patient who is a candidate to be treated with one or more TGF-beta superfamily heteromultimer complexes of the disclosure and an appropriate dosing regimen established for that patient based on the baseline value(s). Alternatively, established baseline parameters based on a patient's medical history could be used to inform an appropriate dosing regimen for a patient. For example, if a healthy patient has an established baseline blood pressure reading that is above the defined normal range it may not be necessary to bring the patient's blood pressure into the range that is considered normal for the general population prior to treatment with the one or more TGF-beta superfamily heteromultimer complexes of the disclosure. A patient's baseline values for one or more hematologic parameters prior to treatment with one or more TGF-beta superfamily heteromultimer complexes of the disclosure may also be used as the relevant comparative values for monitoring any changes to the hematologic parameters during treatment with the one or more TGF-beta superfamily heteromultimer complexes of the disclosure.

In certain embodiments, one or more hematologic parameters are measured in patients who are being treated with a one or more TGF-beta superfamily heteromultimer complexes of the disclosure. The hematologic parameters may be used to monitor the patient during treatment and permit adjustment or termination of the dosing with the one or more TGF-beta superfamily heteromultimer complexes of the disclosure or additional dosing with another therapeutic agent. For example, if administration of one or more TGF-beta superfamily heteromultimer complexes of the disclosure of the disclosure results in an increase in blood pressure, red blood cell level, or hemoglobin level, or a reduction in iron stores, then the dose of the one or more TGF-beta superfamily heteromultimer complexes of the disclosure may be reduced in amount or frequency in order to decrease the effects of the one or more TGF-beta superfamily heteromultimer complexes of the disclosure on the one or more hematologic parameters. If administration of one or more TGF-beta superfamily heteromultimer complexes of the disclosure results in a change in one or more hematologic parameters that is adverse to the patient, then the dosing of the one or more TGF-beta superfamily heteromultimer complexes of the disclosure may be terminated either temporarily, until the hematologic parameter(s) return to an acceptable level, or permanently. Similarly, if one or more hematologic parameters are not brought within an acceptable range after reducing the dose or frequency of administration of the one or more TGF-beta superfamily heteromultimer complexes of the disclosure, then the dosing may be terminated. As an alternative, or in addition to, reducing or terminating the dosing with the one or more TGF-beta superfamily heteromultimer complexes of the disclosure, the patient may be dosed with an additional therapeutic agent that addresses the undesirable level in the hematologic parameter(s), such as, for example, a blood pressure-lowering agent or an iron supplement. For example, if a patient being treated with one or more TGF-beta superfamily heteromultimer complexes of the disclosure has elevated blood pressure, then dosing with the one or more TGF-beta superfamily heteromultimer complexes of the disclosure may continue at the same level and a blood pressure-lowering agent is added to the treatment regimen, dosing with the one or more TGF-beta superfamily heteromultimer complexes of the disclosure may be reduced (e.g., in amount and/or frequency) and a blood pressure-lowering agent is added to the treatment regimen, or dosing with the one or more TGF-beta superfamily heteromultimer complexes of the disclosure may be terminated and the patient may be treated with a blood pressure-lowering agent.

### 6. Pharmaceutical Compositions

In certain aspects, TGF-beta superfamily heteromultimer complexes of the present disclosure can be administered alone or as a component of a pharmaceutical formulation (also referred to as a therapeutic composition or pharmaceutical composition). A pharmaceutical formation refers to a preparation which is in such form as to permit the biological activity of an active ingredient (e.g., an agent of the present disclosure) contained therein to be effective and which contains no additional components which are unacceptably toxic to a subject to which the formulation would be administered. The subject compounds may be formulated for administration in any convenient way for use in human or veterinary medicine. For example, one or more agents of the present disclosure may be formulated with a pharmaceutically acceptable carrier. A pharmaceutically acceptable carrier refers to an ingredient in a pharmaceutical formulation, other than an active ingredient, which is generally nontoxic to a subject. A pharmaceutically acceptable carrier includes, but is not limited to, a buffer, excipient, stabilizer, and/or preservative. In general, pharmaceutical formulations for use in the present disclosure are in a pyrogen-free, physiologically-acceptable form when administered to a subject. Therapeutically useful agents other than those described herein, which may optionally be included in the formulation as described above, may be administered in combination with the subject agents in the methods of the present disclosure.

In certain embodiments, compositions will be administered parenterally [e.g., by intravenous (I.V.) injection, intraarterial injection, intraosseous injection, intramuscular injection, intrathecal injection, subcutaneous injection, or intradermal injection]. Pharmaceutical compositions suitable for parenteral administration may comprise one or more agents of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use. Injectable solutions or dispersions may contain antioxidants, buffers, bacteriostats, suspending agents, thickening agents, or solutes which render the formulation isotonic with the blood of the intended recipient. Examples of suitable aqueous and nonaqueous carriers which may be employed in the pharmaceutical formulations of the present disclosure include water, ethanol, polyols (e.g., glycerol, propylene glycol, polyethylene glycol, etc.), vegetable oils (e.g., olive oil), injectable organic esters (e.g., ethyl oleate), and suitable mixtures thereof. Proper fluidity can be maintained, for example, by the use of coating materials (e.g., lecithin), by the maintenance of the required particle size in the case of dispersions, and by the use of surfactants.

In some embodiments, a therapeutic method of the present disclosure includes administering the pharmaceutical composition systemically, or locally, from an implant or device. Further, the pharmaceutical composition may be encapsulated or injected in a form for delivery to a target tissue site (e.g., bone marrow or muscle). In certain embodiments, compositions of the present disclosure may include a matrix capable of delivering one or more of the agents of the present disclosure to a target tissue site (e.g., bone marrow or muscle), providing a structure for the developing tissue and optimally capable of being resorbed into the body. For example, the matrix may provide slow release of one or more agents of the present disclosure. Such matrices may be formed of materials presently in use for other implanted medical applications.

The choice of matrix material may be based on one or more of: biocompatibility, biodegradability, mechanical properties, cosmetic appearance, and interface properties. The particular application of the subject compositions will define the appropriate formulation. Potential matrices for the compositions may be biodegradable and chemically defined calcium sulfate, tricalciumphosphate, hydroxyapatite, polylactic acid, and polyanhydrides. Other potential materials are biodegradable and biologically well-defined including, for example, bone or dermal collagen. Further matrices are comprised of pure proteins or extracellular matrix components. Other potential matrices are non-biodegradable and chemically defined including, for example, sintered hydroxyapatite, bioglass, aluminates, or other ceramics. Matrices may be comprised of combinations of any of the above mentioned types of material including, for example, polylactic acid and hydroxyapatite or collagen and tricalciumphosphate. The bioceramics may be altered in composition (e.g., calcium-aluminate-phosphate) and processing to alter one or more of pore size, particle size, particle shape, and biodegradability.

In certain embodiments, pharmaceutical compositions of present disclosure can be administered topically. “Topical application” or “topically” means contact of the pharmaceutical composition with body surfaces including, for example, the skin, wound sites, and mucous membranes. The topical pharmaceutical compositions can have various application forms and typically comprises a drug-containing layer, which is adapted to be placed near to or in direct contact with the tissue upon topically administering the composition. Pharmaceutical compositions suitable for topical administration may comprise one or more one or more TGFβ superfamily type I and/or type II receptor polypeptide complexes of the disclosure in combination formulated as a liquid, a gel, a cream, a lotion, an ointment, a foam, a paste, a putty, a semi-solid, or a solid. Compositions in the liquid, gel, cream, lotion, ointment, foam, paste, or putty form can be applied by spreading, spraying, smearing, dabbing or rolling the composition on the target tissue. The compositions also may be impregnated into sterile dressings, transdermal patches, plasters, and bandages. Compositions of the putty, semi-solid or solid forms may be deformable. They may be elastic or non-elastic (e.g., flexible or rigid). In certain aspects, the composition forms part of a composite and can include fibers, particulates, or multiple layers with the same or different compositions.

Topical compositions in the liquid form may include pharmaceutically acceptable solutions, emulsions, microemulsions, and suspensions. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof].

Topical gel, cream, lotion, ointment, semi-solid or solid compositions may include one or more thickening agents, such as a polysaccharide, synthetic polymer or protein-based polymer. In one embodiment of the invention, the gelling agent herein is one that is suitably nontoxic and gives the desired viscosity. The thickening agents may include polymers, copolymers, and monomers of: vinylpyrrolidones, methacrylamides, acrylamides N-vinylimidazoles, carboxy vinyls, vinyl esters, vinyl ethers, silicones, polyethyleneoxides, polyethyleneglycols, vinylalcohols, sodium acrylates, acrylates, maleic acids, NN-dimethylacrylamides, diacetone acrylamides, acrylamides, acryloyl morpholine, pluronic, collagens, polyacrylamides, polyacrylates, polyvinyl alcohols, polyvinylenes, polyvinyl silicates, polyacrylates substituted with a sugar (e.g., sucrose, glucose, glucosamines, galactose, trehalose, mannose, or lactose), acylamidopropane sulfonic acids, tetramethoxyorthosilicates, methyltrimethoxyorthosilicates, tetraalkoxyorthosilicates, trialkoxyorthosilicates, glycols, propylene glycol, glycerine, polysaccharides, alginates, dextrans, cyclodextrin, celluloses, modified celluloses, oxidized celluloses, chitosans, chitins, guars, carrageenans, hyaluronic acids, inulin, starches, modified starches, agarose, methylcelluloses, plant gums, hylaronans, hydrogels, gelatins, glycosaminoglycans, carboxymethyl celluloses, hydroxyethyl celluloses, hydroxy propyl methyl celluloses, pectins, low-methoxy pectins, cross-linked dextrans, starch-acrylonitrile graft copolymers, starch sodium polyacrylate, hydroxyethyl methacrylates, hydroxyl ethyl acrylates, polyvinylene, polyethylvinylethers, polymethyl methacrylates, polystyrenes, polyurethanes, polyalkanoates, polylactic acids, polylactates, poly(3-hydroxybutyrate), sulfonated hydrogels, AMPS (2-acrylamido-2-methyl-1-propanesulfonic acid), SEM (sulfoethylmethacrylate), SPM (sulfopropyl methacrylate), SPA (sulfopropyl acrylate), N,N-dimethyl-N-methacryloxyethyl-N-(3-sulfopropyl)ammonium betaine, methacryllic acid amidopropyl-dimethyl ammonium sulfobetaine, SPI (itaconic acid-bis(1-propyl sulfonizacid-3) ester di-potassium salt), itaconic acids, AMBC (3-acrylamido-3-methylbutanoic acid), beta-carboxyethyl acrylate (acrylic acid dimers), and maleic anhydride-methylvinyl ether polymers, derivatives thereof, salts thereof, acids thereof, and combinations thereof. In certain embodiments, pharmaceutical compositions of present disclosure can be administered orally, for example, in the form of capsules, cachets, pills, tablets, lozenges (using a flavored basis such as sucrose and acacia or tragacanth), powders, granules, a solution or a suspension in an aqueous or non-aqueous liquid, an oil-in-water or water-in-oil liquid emulsion, or an elixir or syrup, or pastille (using an inert base, such as gelatin and glycerin, or sucrose and acacia), and/or a mouth wash, each containing a predetermined amount of a compound of the present disclosure and optionally one or more other active ingredients. A compound of the present disclosure and optionally one or more other active ingredients may also be administered as a bolus, electuary, or paste.

In solid dosage forms for oral administration (e.g., capsules, tablets, pills, dragees, powders, and granules), one or more compounds of the present disclosure may be mixed with one or more pharmaceutically acceptable carriers including, for example, sodium citrate, dicalcium phosphate, a filler or extender (e.g., a starch, lactose, sucrose, glucose, mannitol, and silicic acid), a binder (e.g. carboxymethylcellulose, an alginate, gelatin, polyvinyl pyrrolidone, sucrose, and acacia), a humectant (e.g., glycerol), a disintegrating agent (e.g., agar-agar, calcium carbonate, potato or tapioca starch, alginic acid, a silicate, and sodium carbonate), a solution retarding agent (e.g. paraffin), an absorption accelerator (e.g. a quaternary ammonium compound), a wetting agent (e.g., cetyl alcohol and glycerol monostearate), an absorbent (e.g., kaolin and bentonite clay), a lubricant (e.g., a talc, calcium stearate, magnesium stearate, solid polyethylene glycols, sodium lauryl sulfate), a coloring agent, and mixtures thereof. In the case of capsules, tablets, and pills, the pharmaceutical formulation (composition) may also comprise a buffering agent. Solid compositions of a similar type may also be employed as fillers in soft and hard-filled gelatin capsules using one or more excipients including, e.g., lactose or a milk sugar as well as a high molecular-weight polyethylene glycol.

Liquid dosage forms for oral administration of the pharmaceutical composition may include pharmaceutically acceptable emulsions, microemulsions, solutions, suspensions, syrups, and elixirs. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof]. Besides inert diluents, the oral formulation can also include an adjuvant including, for example, a wetting agent, an emulsifying and suspending agent, a sweetening agent, a flavoring agent, a coloring agent, a perfuming agent, a preservative agent, and combinations thereof.

Suspensions, in addition to the active compounds, may contain suspending agents including, for example, an ethoxylated isostearyl alcohol, polyoxyethylene sorbitol, a sorbitan ester, microcrystalline cellulose, aluminum metahydroxide, bentonite, agar-agar, tragacanth, and combinations thereof.

Prevention of the action and/or growth of microorganisms may be ensured by the inclusion of various antibacterial and antifungal agents including, for example, paraben, chlorobutanol, and phenol sorbic acid.

In certain embodiments, it may be desirable to include an isotonic agent including, for example, a sugar or sodium chloride into the compositions. In addition, prolonged absorption of an injectable pharmaceutical form may be brought about by the inclusion of an agent that delay absorption including, for example, aluminum monostearate and gelatin.

It is understood that the dosage regimen will be determined by the attending physician considering various factors which modify the action of the one or more of the agents of the present disclosure. In the case of a TGF-beta superfamily heteromultimer complex that promotes red blood cell formation, various factors may include, but are not limited to, the patient's red blood cell count, hemoglobin level, the desired target red blood cell count, the patient's age, the patient's sex, the patient's diet, the severity of any disease that may be contributing to a depressed red blood cell level, the time of administration, and other clinical factors. The addition of other known active agents to the final composition may also affect the dosage. Progress can be monitored by periodic assessment of one or more of red blood cell levels, hemoglobin levels, reticulocyte levels, and other indicators of the hematopoietic process.

In certain embodiments, the present disclosure also provides gene therapy for the in vivo production of one or more of the agents of the present disclosure. Such therapy would achieve its therapeutic effect by introduction of the agent sequences into cells or tissues having one or more of the disorders as listed above. Delivery of the agent sequences can be achieved, for example, by using a recombinant expression vector such as a chimeric virus or a colloidal dispersion system. Preferred therapeutic delivery of one or more of agent sequences of the disclosure is the use of targeted liposomes.

Various viral vectors which can be utilized for gene therapy as taught herein include adenovirus, herpes virus, vaccinia, or an RNA virus (e.g., a retrovirus). The retroviral vector may be a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing one or more of the agents of the present disclosure.

Alternatively, tissue culture cells can be directly transfected with plasmids encoding the retroviral structural genes (gag, pol, and env), by conventional calcium phosphate transfection. These cells are then transfected with the vector plasmid containing the genes of interest. The resulting cells release the retroviral vector into the culture medium.

Another targeted delivery system for one or more of the agents of the present disclosure is a colloidal dispersion system. Colloidal dispersion systems include, for example, macromolecule complexes, nanocapsules, microspheres, beads, and lipid-based systems including oil-in-water emulsions, micelles, mixed micelles, and liposomes. In certain embodiments, the preferred colloidal system of this disclosure is a liposome. Liposomes are artificial membrane vesicles which are useful as delivery vehicles in vitro and in vivo. RNA, DNA, and intact virions can be encapsulated within the aqueous interior and be delivered to cells in a biologically active form. See, e.g., Fraley, et al. (1981) Trends Biochem. Sci., 6:77. Methods for efficient gene transfer using a liposome vehicle are known in the art. See, e.g., Mannino, et al. (1988) Biotechniques, 6:682, 1988.

The composition of the liposome is usually a combination of phospholipids, which may include a steroid (e.g. cholesterol). The physical characteristics of liposomes depend on pH, ionic strength, and the presence of divalent cations. Other phospholipids or other lipids may also be used including, for example a phosphatidyl compound (e.g., phosphatidylglycerol, phosphatidylcholine, phosphatidylserine, phosphatidylethanolamine, a sphingolipid, a cerebroside, and a ganglioside), egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

### EXEMPLIFICATION

The invention now being generally described, it will be more readily understood by reference to the following examples, which are included merely for purposes of illustration of certain embodiments and embodiments of the present invention, and are not intended to limit the invention.

### Example 1. Generation of an ActRIIB-Fc:ALK4-Fc Heterodimer

Applicants constructed a soluble ActRIIB-Fc:ALK4-Fc heteromeric complex comprising the extracellular domains of human ActRIIB and human ALK4, which are each separately fused to an Fc domain with a linker positioned between the extracellular domain and the Fc domain. The individual constructs are referred to as ActRIIB-Fc fusion polypeptide and ALK4-Fc fusion polypeptide, respectively, and the sequences for each are provided below.

A methodology for promoting formation of ActRIIB-Fc:ALK4-Fc heteromeric complexes, as opposed to the ActRIIB-Fc or ALK4-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIB-Fc and ALK4-Fc polypeptide sequences of SEQ ID NOs: 100-102 and 104-106, respectively, one Fc domain is altered to introduce cationic amino acids at the interaction face, while the other Fc domain is altered to introduce anionic amino acids at the interaction face. The ActRIIB-Fc fusion polypeptide and ALK4-Fc fusion polypeptide each employ the tissue plasminogen activator (TPA) leader:

(SEQ ID NO: 98)

MDAMKRGLCCVLLLCGAVFVSP.

The ActRIIB-Fc polypeptide sequence (SEQ ID NO: 100) is shown below:

| (SEQ ID NO: 100) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSRKEMT KNQVSLTCLV KGFYPSDIAV |
| 301 | EWESNGQPEN NYKTTPPVLK SDGSFFLYSK LTVDKSRWQQ GNVFSCSVMH |
| 351 | EALHNHYTQK SLSLSPGK |

The leader (signal) sequence and linker are underlined. To promote formation of the ActRIIB-Fc:ALK4-Fc heterodimer rather than either of the possible homodimeric complexes, two amino acid substitutions (replacing acidic amino acids with lysine) can be introduced into the Fc domain of the ActRIIB fusion protein as indicated by double underline above. The amino acid sequence of SEQ ID NO: 100 may optionally be provided with lysine (K) removed from the C-terminus.

This ActRIIB-Fc fusion protein is encoded by the following nucleic acid sequence (SEQ ID NO: 101):

| (SEQ ID NO: 101) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGAA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGAAG TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAA |

The mature ActRIIB-Fc fusion polypeptide (SEQ ID NO: 102) is as follows, and may optionally be provided with lysine removed from the C-terminus.

| (SEQ ID NO: 102) | |
|---|---|
| 1 | GRGEAETREC IYYNANWELE RTNQSGLERC EGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | RKEMTKNQVS LTCLVKGFYP SDIAVEWESN GQPENNYKTT PPVLKSDGSF |
| 301 | FLYSKLTVDK SRWQQGNVFS CSVMHEALHN HYTQKSLSLS PGK |

The complementary form of ALK4-Fc fusion polypeptide (SEQ ID NO: 104) is as follows:

| (SEQ ID NO: 104) | |
|---|---|
| 1 | MDAMKRGLCC VLLLCGAVFV SPGASGPRGV QALLCACTSC LQANYTCETD |
| 51 | GACMVSIFNL DGMEHHVRTC IPKVELVPAG KPFYCLSSED LRNTHCCYTD |
| 101 | YCNRIDLRVP SGHLKEPEHP SMWGPVETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVYTL PPSREEMTKN QVSLTCLVKG FYPSDIAVEW ESNGQPENNY |
| 301 | DTTPPVLDSD GSFFLYSDLT VDKSRWQQGN VFSCSVMHEA LHNHYTQKSL |
| 351 | SLSPG |

The leader sequence and linker sequence are underlined. To guide heterodimer formation with the ActRIIB-Fc fusion polypeptide of SEQ ID NOs: 100 and 102 above, two amino acid substitutions (replacing lysines with aspartic acids) can be introduced into the Fc domain of the ALK4-Fc fusion polypeptide as indicated by double underline above. The amino acid sequence of SEQ ID NO: 104 may optionally be provided with lysine added at the C-terminus.

This ALK4-Fc fusion protein is encoded by the following nucleic acid (SEQ ID NO:

| (SEQ ID NO: 105) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCCGGGCC CCGGGGGGTC CAGGCTCTGC |
| 101 | TGTGTGCGTG CACCAGCTGC CTCCAGGCCA ACTACACGTG TGAGACAGAT |
| 151 | GGGGCCTGCA TGGTTTCCAT TTTCAATCTG GATGGGATGG AGCACCATGT |
| 201 | GCGCACCTGC ATCCCCAAAG TGGAGCTGGT CCCTGCCGGG AAGCCCTTCT |
| 251 | ACTGCCTGAG CTCGGAGGAC CTGCGCAACA CCCACTGCTG CTACACTGAC |
| 301 | TACTGCAACA GGATCGACTT GAGGGTGCCC AGTGGTCACC TCAAGGAGCC |
| 351 | TGAGCACCCG TCCATGTGGG GCCCGGTGGA GACCGGTGGT GGAACTCACA |
| 401 | CATGCCCACC GTGCCCAGCA CCTGAACTCC TGGGGGGACC GTCAGTCTTC |
| 451 | CTCTTCCCCC CAAAACCCAA GGACACCCTC ATGATCTCCC GGACCCCTGA |
| 501 | GGTCACATGC GTGGTGGTGG ACGTGAGCCA CGAAGACCCT GAGGTCAAGT |
| 551 | TCAACTGGTA CGTGGACGGC GTGGAGGTGC ATAATGCCAA GACAAAGCCG |
| 601 | CGGGAGGAGC AGTACAACAG CACGTACCGT GTGGTCAGCG TCCTCACCGT |
| 651 | CCTGCACCAG GACTGGCTGA ATGGCAAGGA GTACAAGTGC AAGGTCTCCA |
| 701 | ACAAAGCCCT CCCAGCCCCC ATCGAGAAAA CCATCTCCAA AGCCAAAGGG |
| 751 | CAGCCCCGAG AACCACAGGT GTACACCCTG CCCCCATCCC GGGAGGAGAT |
| 801 | GACCAAGAAC CAGGTCAGCC TGACCTGCCT GGTCAAAGGC TTCTATCCCA |
| 851 | GCGACATCGC CGTGGAGTGG GAGAGCAATG GGCAGCCGGA GAACAACTAC |
| 901 | GACACCACGC CTCCCGTGCT GGACTCCGAC GGCTCCTTCT TCCTCTATAG |
| 951 | CGACCTCACC GTGGACAAGA GCAGGTGGCA GCAGGGGAAC GTCTTCTCAT |
| 1001 | GCTCCGTGAT GCATGAGGCT CTGCACAACC ACTACACGCA GAAGAGCCTC |
| 1051 | TCCCTGTCTC CGGGT |

The mature ALK4-Fc fusion protein sequence (SEQ ID NO: 106) is as follows and may optionally be provided with lysine added at the C-terminus.

| (SEQ ID NO: 106) | |
|---|---|
| 1 | SGPRGVQALL CACTSCLQAN YTCETDGACM VSIFNLDGME HHVRTCIPKV |
| 51 | ELVPAGKPFY CLSSEDLRNT HCCYTDYCNR IDLRVPSGHL KEPEHPSMWG |
| 101 | PVETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVYTLPPSR EEMTKNQVSL |
| 251 | TCLVKGFYPS DIAVEWESNG QPENNYDTTP PVLDSDGSFF LYSDLTVDKS |
| 301 | RWQQGNVFSC SVMHEALHNH YTQKSLSLSP G |

The ActRIIB-Fc and ALK4-Fc proteins of SEQ ID NO: 102 and SEQ ID NO: 106, respectively, may be co-expressed and purified from a CHO cell line, to give rise to a heteromeric complex comprising ActRIIB-Fc:ALK4-Fc.

In another approach to promote the formation of heteromultimer complexes using asymmetric Fc fusion proteins the Fc domains are altered to introduce complementary hydrophobic interactions and an additional intermolecular disulfide bond as illustrated in the ActRIIB-Fc and ALK4-Fc polypeptide sequences of SEQ ID NOs: 401-402 and 403-404, respectively. The ActRIIB-Fc fusion polypeptide and ALK4-Fc fusion polypeptide each employ the tissue plasminogen activator (TPA) leader: MDAMKRGLCCVLLLCGAVFVSP (SEQ ID NO: 98).

The ActRIIB-Fc polypeptide sequence (SEQ ID NO: 401) is shown below:

| (SEQ ID NO: 401) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPCREEMT KNQVSLWCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

The leader (signal) sequence and linker sequence are underlined. To promote formation of the ActRIIB-Fc:ALK4-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 401 may optionally be provided with lysine removed from the C-terminus.

The mature ActRIIB-Fc fusion polypeptide is as follows:

| (SEQ ID NO: 402) | |
|---|---|
| 1 | GRGEAETREC IYYNANWELE RTNQSGLERC EGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPC |
| 251 | REEMTKNQVS LWCLVKGFYP SDIAVEWESN GQPENNYKTT PPVLDSDGSF |
| 301 | FLYSKLTVDK SRWQQGNVFS CSVMHEALHN HYTQKSLSLS PGK |

The complementary form of ALK4-Fc fusion polypeptide (SEQ ID NO: 403) is as follows and may optionally be provided with lysine removed from the C-terminus.

| (SEQ ID NO: 403) | |
|---|---|
| 1 | MDAMKRGLCC VLLLCGAVFV SPGASGPRGV QALLCACTSC LQANYTCETD |
| 51 | GACMVSIFNL DGMEHHVRTC IPKVELVPAG KPFYCLSSED LRNTHCCYTD |
| 101 | YCNRIDLRVP SGHLKEPEHP SMWGPVETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVCTL PPSREEMTKN QVSLSCAVKG FYPSDIAVEW ESNGQPENNY |
| 301 | KTTPPVLDSD GSFFLVSKLT VDKSRWQQGN VFSCSVMHEA LHNHYTQKSL |
| 351 | SLSPGK |

The leader sequence and the linker are underlined. To guide heterodimer formation with the ActRIIB-Fc fusion polypeptide of SEQ ID NOs: 401 and 402 above, four amino acid substitutions can be introduced into the Fc domain of the ALK4 fusion polypeptide as indicated by double underline above. The amino acid sequence of SEQ ID NO: 403 may optionally be provided with lysine removed from the C-terminus.

The mature ALK4-Fc fusion protein sequence is as follows and may optionally be provided with lysine removed from the C-terminus.

| (SEQ ID NO: 404) | |
|---|---|
| 1 | SGPRGVQALL CACTSCLQAN YTCETDGACM VSIFNLDGME HHVRTCIPKV |
| 51 | ELVPAGKPFY CLSSEDLRNT HCCYTDYCNR IDLRVPSGHL KEPEHPSMWG |
| 101 | PVETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVCTLPPSR EEMTKNQVSL |
| 251 | SCAVKGFYPS DIAVEWESNG QPENNYKTTP PVLDSDGSFF LVSKLTVDKS |
| 301 | RWQQGNVFSC SVMHEALHNH YTQKSLSLSP GK |

The ActRIIB-Fc and ALK4-Fc proteins of SEQ ID NO: 402 and SEQ ID NO: 404, respectively, may be co-expressed and purified from a CHO cell line, to give rise to a heteromeric complex comprising ActRIITB-Fc:ALK4-Fc.

Purification of various ActRIIB-Fc:ALK4-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 2. Ligand Binding Profile of ActRIIB-Fc:ALK4-Fc Heterodimer Compared to ActRIIB-Fc Homodimer and ALK4-Fc Homodimer

A Biacore™-based binding assay was used to compare ligand binding selectivity of the ActRIIB-Fc:ALK4-Fc heterodimeric complex described above with that of ActRIIB-Fc and ALK4-Fc homodimeric complexes. The ActRIIB-Fc:ALK4-Fc heterodimer, ActRIIB-Fc homodimer, and ALK4-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand off-rate is a particularly significant parameter to evaluate for ligand traps. Soluble receptor-Fc proteins administered in vivo are in constant competition with native receptors for ligands. When endogenous ligands of the TGFbeta superfamily typically bind to cognate receptors at the cell surface, a multi-step signal transduction process is triggered that is relatively slow on a molecular time scale. Native receptors dissociate from ligand slowly in part because significant time is required to generate an intracellular signal from a ligand binding event. For a soluble receptor-Fc protein to compete effectively for ligand, the off-rate for its complex with the ligand needs to be similar to, or slower than, the off-rate for a ligand complex with native receptor. Ligand binding is a dynamic process and some fraction of ligands will always be in unbound form, so it is important therapeutically for a dose of receptor-Fc protein to capture target ligand for as long as possible. One way to shift the binding equilibrium in favor of more captured ligand is to increase the concentration (dose level) of inhibitor, however this can generate off-target effects that reduce tolerability and safety. A preferable approach is to use an inhibitor with a slower ligand off-rate (longer capture time) combined with ligand binding selectivity to achieve an effective level of ligand antagonism at a lower concentration of inhibitor.

Ligand binding profile of ActRIIB-Fc:ALK4-Fc heterodimer
compared to ActRIIB-Fc homodimer and ALK4-Fc homodimer

| | ActRIIB-Fc | | | ALK4-Fc | | | ActRIIB-Fc:ALK4-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |
| Activin A | 1.2 × 10⁷ | 2.3 × 10⁻⁴ | 19 | 5.8 × 10⁵ | 1.2 × 10⁻² | 20000 | 1.3 × 10⁷ | 1.5 × 10⁻⁴ | 12 |
| Activin B | 5.1 × 10⁶ | 1.0 × 10⁻⁴ | 20 | No binding | | | 7.1 × 10⁶ | 4.0 × 10⁻⁵ | 6 |
| BMP6 | 3.2 × 10⁷ | 6.8 × 10⁻³ | 190 | — | | | 2.0 × 10⁶ | 5.5 × 10⁻³ | 2700 |
| BMP9 | 1.4 × 10⁷ | 1.1 × 10⁻³ | 77 | — | | | Transient* | | 3400 |
| BMP10 | 2.3 × 10⁷ | 2.6 × 10⁻⁴ | 11 | — | | | 5.6 × 10⁷ | 4.1 × 10⁻³ | 74 |
| GDF3 | 1.4 × 10⁶ | 2.2 × 10⁻³ | 1500 | — | | | 3.4 × 10⁶ | 1.7 × 10⁻² | 4900 |
| GDF8 | 8.3 × 10⁵ | 2.3 × 10⁻⁴ | 280 | 1.3 × 10⁵ | 1.9 × 10⁻³ | 15000† | 3.9 × 10⁵ | 2.1 × 10⁻⁴ | 550 |
| GDF11 | 5.0 × 10⁷ | 1.1 × 10⁻⁴ | 2 | 5.0 × 10⁶ | 4.8 × 10⁻³ | 270† | 3.8 × 10⁷ | 1.1 × 10⁻⁴ | 3 |

*Indeterminate due to transient nature of interaction
†Very low signal
— Not tested

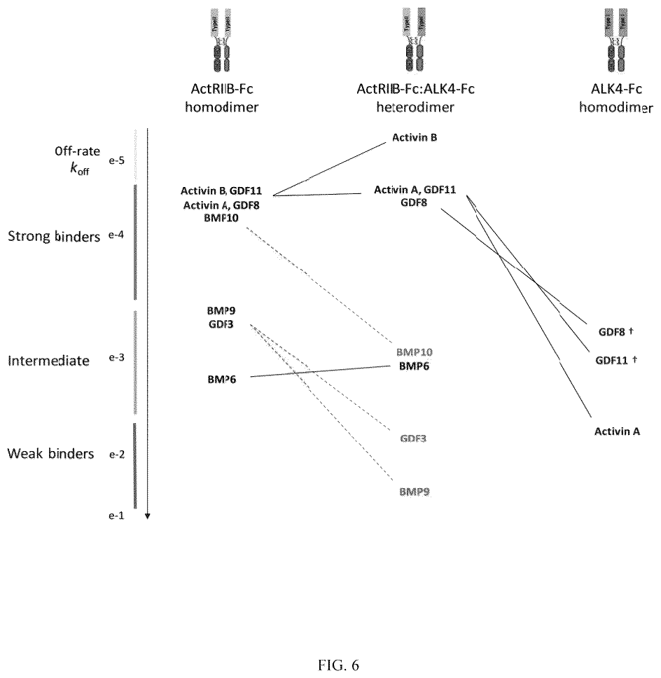

These comparative binding data demonstrate that the ActRIIB-Fc:ALK4-Fc heterodimer has an altered binding profile/selectivity relative to either the ActRIIB-Fc or ALK4-Fc homodimers. The ActRIIB-Fc:ALK4-Fc heterodimer displays enhanced binding to activin B compared with either homodimer, retains strong binding to activin A, GDF8, and GDF11 as observed with ActRIIB-Fc homodimer, and exhibits substantially reduced binding to BMP9, BMP10, and GDF3. In particular, BMP9 displays low or no observable affinity for the ActRIIB-Fc:ALK4-Fc heterodimer, whereas this ligand binds strongly to ActRIIB-Fc homodimer. Like ActRIIB-Fc homodimer, the heterodimer retains intermediate-level binding to BMP6. See FIG. 6.

These results therefore demonstrate that the ActRIIB-Fc:ALK4-Fc heterodimer is a more selective antagonist of activin A, activin B, GDF8, and GDF11 compared to a ActRIIB-Fc homodimer. Accordingly, an ActRIIB-Fc:ALK4-Fc heterodimer will be more useful than an ActRIIB-Fc homodimer in certain applications where such selective antagonism is advantageous. Examples include therapeutic applications where it is desirable to retain antagonism of one or more of activin A, activin B, activin AC, GDF8, and GDF11 but minimize antagonism of one or more of BMP9, BMP10, and BMP6.

### Example 3. Activity Profile of ActRIIB-Fc:ALK4-Fc Heterodimer in Mice Compared to ActRIIB-Fc Homodimer

Homodimeric and heterodimeric complexes were tested in mice to investigate differences in their activity profiles in vivo. Wild-type C₅₇BL/6 mice were dosed subcutaneously with an ActRIIB-Fc homodimer (10 mg/kg), an ActRIIB-Fc:ALK4-Fc heterodimer (3 or 10 mg/kg), or vehicle (phosphate-buffered saline, PBS) twice per week for 4 weeks beginning at approximately 10 weeks of age (n=9 mice per group). ALK4-Fc homodimer was not tested in vivo due to its inability to bind ligands with high affinity under cell-free conditions as determined by surface plasmon resonance. Study endpoints included: body weight; total lean mass and total adipose mass as determined by nuclear magnetic resonance (NVR) at baseline and study completion (4 weeks); total bone mineral density as determined by dual energy x-ray absorptiometry (DEXA) at baseline and 4 weeks; and weights of the gastrocnemius, rectus femoris, and pectoralis muscles determined at 4 weeks.

Activity of ActRIIB-Fc and ALK4-Fc
Complexes in Wild-Type Mice

| | | ActRIIB-Fc | ActRIIB-Fc:ALK4-Fc | |
|---|---|---|---|---|
| Endpoint | | homodimer | heterodimer | |
| (4 wk) | Vehicle | 10 mg/kg | 10 mg/kg | 3 mg/kg |
| Change in body weight | ↑ 15% | ↑ 38% ** | ↑ 41% ** | ↑ 33% ** |
| from baseline | | | | |
| Change in total lean | ↓ 1% | ↑ 5% ** | ↑ 5% ** | ↑ 5% ** |
| mass from baseline | | | | |
| Change in total | ↑ 5% | ↓ 36% ** | ↓ 35% ** | ↓ 35% ** |
| adipose mass from | | | | |
| baseline | | | | |
| Change in total bone | ↑ 8% | ↑ 14% * | ↑ 12% * | ↑ 11% |
| mineral density from | | | | |
| baseline | | | | |
| Gastrocnemius weight † | 23 | 36 ** | 35 ** | 30 ** |
| Femoris weight † | 11.5 | 17 ** | 16 ** | 14 ** |
| Pectoralis weight † | 15 | 23 ** | 28 ** | 23 ** |

* P < 0.05 vs. vehicle
** P < 0.01 vs. vehicle
† Combined left and right muscle weights normalized to femur length (mg/mm) to control for body size

Study results are summarized in the table above. As expected, ActRIIB-Fc homodimer caused marked changes in body composition, many consistent with known effects of GDF8 and activin inhibition. Treatment of wild-type mice with ActRIIB-Fc homodimer more than doubled body weight gain over the course of the study compared to vehicle-treated controls.

Accompanying this net weight gain were signficant increases in total lean mass and total bone mineral density, as well as a significant reduction in total adipose mass, compared to vehicle. It should be recognized that normalized (percentage-based) changes in lean and adipose tissues differ in their correspondence to absolute changes because lean mass (typically about 70% of body weight in a mouse) is much larger than adipose mass (typically about 10% of body weight). Individual skeletal muscles examined, including the gastrocnemius, femoris, and pectoralis all increased significantly in weight compared to vehicle controls over the course of treatment with ActRIIB-Fc homodimer.

The ActRIIB-Fc:ALK4-Fc heterodimer produced certain effects strikingly similar to those of the ActRIIB-Fc homodimer despite differential ligand selectivity of the two complexes. As shown in the table above, treatment of mice with the ActRIIB-Fc:ALK4-Fc heterodimer at a dose level of 10 mg/kg matched, nearly matched, or exceeded the effects of ActRIIB-Fc homodimer at the same dose level for all endpoints listed. Effects of the ActRIIB-Fc:ALK4-Fc heterodimer at 3 mg/kg were mildly attenuated for several endpoints compared to 10 mg/kg, thus providing evidence for a dose-effect relationship.

Thus, an ActRIIB-Fc:ALK4-Fc heterodimer exerts beneficial anabolic effects on skeletal muscle and bone, and catabolic effects on adipose tissue, very similar to those of ActRIIB-Fc homodimer. However, unlike ActRIIB homodimer, an ActRIIB-Fc:ALK4-Fc heterodimer exhibits only low-affinity or transient binding to BMP9 and BMP10 and so will not concurrently inhibit processes mediated by those ligands, such as angiogenesis. This novel selectivity will be useful, for example, in treating patients in need of stimulatory effects on muscle and bone, and inhibitory effects on fat, but not in need of altered angiogenesis.

### Example 4. Generation of an ActRIIB-Fc:ALK3-Fc Heterodimer

Applicants constructed a soluble ActRIIB-Fc:ALK3-Fc heteromeric complex comprising the extracellular domains of human ActRIIB and human ALK3, which are each fused to an Fc domain with a linker positioned between the extracellular domain and the Fe domain. The individual constructs are referred to as ActRIIB-Fc and ALK3-Fc, respectively.

Formation of heteromeric ActRIIB-Fc:ALK3-Fc may be guided by approaches similar to those described in Example 1.

In a first approach, the polypeptide sequence of the ActRIIB-Fc fusion protein and a nucleic acid sequence encoding it are provided above in Example 1 as SEQ ID NOs: 100-102.

The complementary ALK3-Fc fusion protein employs the TPA leader and is as follows:

| (SEQ ID NO: 115) | |
|---|---|
| 1 | MDAMKRGLCC VLLLCGAVFV SPGAQNLDSM LHGTGMKSDS DQKKSENGVT |
| 51 | LAPEDTLPFL KCYCSGHCPD DAINNTCITN GHCFAIIEED DQGETTLASG |
| 101 | CMKYEGSDFQ CKDSPKAQLR RTIECCRTNL CNQYLQPTLP PVVIGPFFDG |
| 151 | SIRTGGGTHT CPPCPAPELL GGPSVFLFPP KPKDTLMISR TPEVTCVVVD |
| 201 | VSHEDPEVKF NWYVDGVEVH NAKTKPREEQ YNSTYRVVSV LTVLHQDWLN |
| 251 | GKEYKCKVSN KALPAPIEKT ISKAKGQPRE PQVYTLPPSR EEMTKNQVSL |
| 301 | TCLVKGFYPS DIAVEWESNG QPENNYDTTP PVLDSDGSFF LYSDLTVDKS |
| 351 | RWQQGNVFSC SVMHEALHNH YTQKSLSLSP G |

The leader and linker sequences are underlined. To promote formation of the ActRIIB-Fc:ALK3-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 115 may optionally be provided with a lysine added at the C-terminus.

This ALK3-Fc fusion protein is encoded by the following nucleic acid (SEQ ID NO: 116).

| (SEQ ID NO: 116) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCCAGAATCT GGATAGTATG CTTCATGGCA |
| 101 | CTGGGATGAA ATCAGACTCC GACCAGAAAA AGTCAGAAAA TGGAGTAACC |
| 151 | TTAGCACCAG AGGATACCTT GCCTTTTTTA AAGTGCTATT GCTCAGGGCA |
| 201 | CTGTCCAGAT GATGCTATTA ATAACACATG CATAACTAAT GGACATTGCT |
| 251 | TTGCCATCAT AGAAGAAGAT GACCAGGGAG AAACCACATT AGCTTCAGGG |
| 301 | TGTATGAAAT ATGAAGGATC TGATTTTCAG TGCAAAGATT CTCCAAAAGC |
| 351 | CCAGCTACGC CGGACAATAG AATGTTGTCG GACCAATTTA TGTAACCAGT |
| 401 | ATTTGCAACC CACACTGCCC CCTGTTGTCA TAGGTCCGTT TTTTGATGGC |
| 451 | AGCATTCGAA CCGGTGGTGG AACTCACACA TGCCCACCGT GCCCAGCACC |
| 501 | TGAACTCCTG GGGGGACCGT CAGTCTTCCT CTTCCCCCCA AAACCCAAGG |
| 551 | ACACCCTCAT GATCTCCCGG ACCCCTGAGG TCACATGCGT GGTGGTGGAC |
| 601 | GTGAGCCACG AAGACCCTGA GGTCAAGTTC AACTGGTACG TGGACGGCGT |
| 651 | GGAGGTGCAT AATGCCAAGA CAAAGCCGCG GGAGGAGCAG TACAACAGCA |
| 701 | CGTACCGTGT GGTCAGCGTC CTCACCGTCC TGCACCAGGA CTGGCTGAAT |
| 751 | GGCAAGGAGT ACAAGTGCAA GGTCTCCAAC AAAGCCCTCC CAGCCCCCAT |
| 801 | CGAGAAAACC ATCTCCAAAG CCAAAGGGCA GCCCCGAGAA CCACAGGTGT |
| 851 | ACACCCTGCC CCCATCCCGG GAGGAGATGA CCAAGAACCA GGTCAGCCTG |
| 901 | ACCTGCCTGG TCAAAGGCTT CTATCCCAGC GACATCGCCG TGGAGTGGGA |
| 951 | GAGCAATGGG CAGCCGGAGA ACAACTACGA CACCACGCCT CCCGTGCTGG |
| 1001 | ACTCCGACGG CTCCTTCTTC CTCTATAGCG ACCTCACCGT GGACAAGAGC |
| 1051 | AGGTGGCAGC AGGGGAACGT CTTCTCATGC TCCGTGATGC ATGAGGCTCT |
| 1101 | GCACAACCAC TACACGCAGA AGAGCCTCTC CCTGTCTCCG GGT |

The mature ALK3-Fc fusion protein sequence is as follows (SEQ ID NO: 117) and may optionally be provided with a lysine added at the C-terminus.

| (SEQ ID NO: 117) | |
|---|---|
| 1 | GAQNLDSMLH GTGMKSDSDQ KKSENGVTLA PEDTLPFLKC YCSGHCPDDA |
| 51 | INNTCITNGH CFAIIEEDDQ GETTLASGCM KYEGSDFQCK DSPKAQLRRT |
| 101 | IECCRTNLCN QYLQPTLPPV VIGPFFDGSI RTGGGTHTCP PCPAPELLGG |
| 151 | PSVFLFPPKP KDTLMISRTP EVTCVVVDVS HEDPEVKFNW YVDGVEVHNA |
| 201 | KTKPREEQYN STYRVVSVLT VLHQDWLNGK EYKCKVSNKA LPAPIEKTIS |
| 251 | KAKGQPREPQ VYTLPPSREE MTKNQVSLTC LVKGFYPSDI AVEWESNGQP |
| 301 | ENNYDTTPPV LDSDGSFFLY SDLTVDKSRW QQGNVFSCSV MHEALHNHYT |
| 351 | QKSLSLSPG |

The ActRIIB-Fc and ALK3-Fc fusion proteins of SEQ ID NO: 102 and SEQ ID NO: 117, respectively, may be co-expressed and purified from a CHO cell line to give rise to a heteromeric complex comprising ActRIIB-Fc:ALK3-Fc.

In another approach to promoting the formation of heteromultimer complexes using asymmetric Fc fusion proteins, illustrated in the ActRIIB-Fc and ALK3-Fc polypeptide sequences of SEQ ID NOs: 401-402 and 407-408, respectively, the Fc domains are altered to introduce complementary hydrophobic interactions and an additional intermolecular disulfide bond. The ActRIIB-Fc fusion polypeptide sequences are discussed in Example 1.

The complementary form of ALK3-Fc fusion polypeptide (SEQ ID NO: 407) is as follows:

| (SEQ ID NO: 407) | |
|---|---|
| 1 | MDAMKRGLCC VLLLCGAVFV SPGAQNLDSM LHGTGMKSDS DQKKSENGVT |
| 51 | LAPEDTLPFL KCYCSGHCPD DAINNTCITN GHCFAIIEED DQGETTLASG |
| 101 | CMKYEGSDFQ CKDSPKAQLR RTIECCRTNL CNQYLQPTLP PVVIGPFFDG |
| 151 | SIRTGGGTHT CPPCPAPELL GGPSVFLFPP KPKDTLMISR TPEVTCVVVD |
| 201 | VSHEDPEVKF NWYVDGVEVH NAKTKPREEQ YNSTYRVVSV LTVLHQDWLN |
| 251 | GKEYKCKVSN KALPAPIEKT ISKAKGQPRE PQVCTLPPSR EEMTKNQVSL |
| 301 | SCAVKGFYPS DIAVEWESNG QPENNYKTTP PVLDSDGSFF LVSKLTVDKS |
| 351 | RWQQGNVFSC SVMHEALHNH YTQKSLSLSP GK |

The leader sequence and linker are underlined. To guide heterodimer formation with the ActRIIB-Fc fusion polypeptide of SEQ ID NOs 401 and 402 above, four amino acid substitutions can be introduced into the Fc domain of the ALK3 fusion polypeptide as indicated by double underline above. The amino acid sequence of SEQ ID NO: 407 may optionally be provided with the lysine removed from the C-terminus.

The mature ALK3-Fc fusion protein sequence (SEQ ID NO: 408) is as follows and may optionally be provided with the lysine (K) removed from the C-terminus.

| (SEQ ID NO: 408) | |
|---|---|
| 1 | GAQNLDSMLH GTGMKSDSDQ KKSENGVTLA PEDTLPFLKC YCSGHCPDDA |
| 51 | INNTCITNGH CFAIIEEDDQ GETTLASGCM KYEGSDFQCK DSPKAQLRRT |
| 101 | IECCRTNLCN QYLQPTLPPV VIGPFFDGSI RTGGGTHTCP PCPAPELLGG |
| 151 | PSVFLFPPKP KDTLMISRTP EVTCVVVDVS HEDPEVKFNW YVDGVEVHNA |
| 201 | KTKPREEQYN STYRVVSVLT VLHQDWLNGK EYKCKVSNKA LPAPIEKTIS |
| 251 | KAKGQPREPQ VCTLPPSREE MTKNQVSLSC AVKGFYPSDI AVEWESNGQP |
| 301 | ENNYKTTPPV LDSDGSFFLV SKLTVDKSRW QQGNVFSCSV MHEALHNHYT |
| 351 | QKSLSLSPGK |

The ActRIIB-Fc and ALK3-Fc proteins of SEQ ID NO: 402 and SEQ ID NO: 408, respectively, may be co-expressed and purified from a CHO cell line, to give rise to a heteromeric complex comprising ActRIITB-Fc:ALK3-Fc.

Purification of various ActRIIB-Fc:ALK3-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 5. Ligand Binding Profile of ActRIIB-Fc:ALK3-Fc Heterodimer Compared to ActRIIB-Fc Homodimer and ALK3-Fc Homodimer

A Biacore™-based binding assay was used to compare ligand binding selectivity of the ActRIIB-Fc:ALK3-Fc heterodimeric complex described above with that of ActRIIB-Fc and ALK3-Fc homodimeric complexes. The ActRIIB-Fc:ALK3-Fc heterodimer, ActRIIB-Fc homodimer, and ALK3-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand binding profile of ActRIIB-Fc:ALK3-Fc heterodimer
compared to ActRIIB-Fc homodimer and ALK3-Fc homodimer

| | ActRIIB-Fc | | | ALK3-Fc | | | ActRIIB-Fc:ALK3-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |
| Activin A | 1.3 × 10⁷ | 1.4 × 10⁻⁴ | 11 | No binding | | | 3.4 × 10⁷ | 5.0 × 10⁻³ | 150 |
| Activin B | 5.1 × 10⁶ | 1.0 × 10⁻⁴ | 20 | No binding | | | 2.8 × 10⁶ | 5.7 × 10⁻⁴ | 200 |
| BMP2 | Transient* | | >66000 | 6.8 × 10⁵ | 8.9 × 10⁻⁵ | 130 | 8.0 × 10⁶ | 1.1 × 10⁻⁵ | 1 |
| BMP4 | — | | | 3.0 × 10⁵ | 5.3 × 10⁻⁵ | 180 | 2.6 × 10⁶ | 6.5 × 10⁻⁶ | 3 |
| BMP5 | 2.6 × 10⁷ | 7.5 × 10⁻² | 2900 | 2.9 × 10⁴ | 2.0 × 10⁻³ | 70000 | 9.0 × 10⁵ | 5.8 × 10⁻⁴ | 640 |
| BMP6 | 3.5 × 10⁷ | 6.8 × 10⁻³ | 190 | 1.4 × 10⁵ | 4.9 × 10⁻³ | 35000 | 2.0 × 10⁷ | 2.9 × 10⁻⁴ | 15 |
| BMP7 | 8.8 × 10⁶ | 1.4 × 10⁻² | 1600 | 1.2 × 10⁶ | 1.8 × 10⁻² | 15000 | 8.2 × 10⁵ | 1.5 × 10⁻³ | 1900 |

| BMP9 | 3.9 × 10⁷ | 1.3 × 10⁻³ | 34 | No binding | Transient* | | >33000 |
|---|---|---|---|---|---|---|---|
| BMP10 | 5.9 × 10⁷ | 2.0 × 10⁻⁴ | 4 | No binding | 3.0 × 10⁷ | 9.4 × 10⁻⁴ | 31 |
| GDF3 | 1.6 × 10⁶ | 2.3 × 10⁻³ | 1400 | No binding | 1.4 × 10⁷ | 8.2 × 10⁻² | 5900 |

| GDF5 | Transient* | >9600 | 4.8 × 10⁵ | 1.1 × 10⁻² | 22000 | 1.2 × 10⁷ | 8.3 × 10⁻⁴ | 70 |
|---|---|---|---|---|---|---|---|---|
| GDF6 | — | | 3.4 × 10⁴ | 1.3 × 10⁻³ | 40000 | 2.8 × 10⁵ | 4.5 × 10⁻⁴ | 1600 |
| GDF7 | Transient* | >12000 | 2.2 × 10⁵ | 2.7 × 10⁻² | 12000 | 7.5 × 10⁶ | 4.0 × 10⁻⁴ | 52 |

| | | | | | | | |
|---|---|---|---|---|---|---|---|
| GDF8 | 8.3 × 10⁵ | 2.3 × 10⁻⁴ | 280 | No binding | 3.0 × 10⁶ | 9.2 × 10⁻⁴ | 310 |
| GDF11 | 5.0 × 10⁷ | 1.1 × 10⁻⁴ | 2 | No binding | 1.6 × 10⁷ | 1.1 × 10⁻³ | 66 |

*Indeterminate due to transient nature of interaction
— Not tested

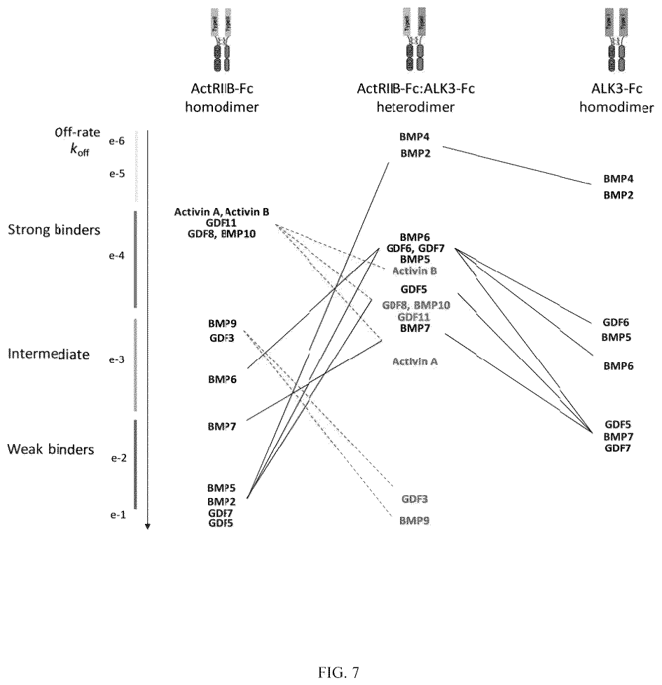

These comparative binding data demonstrate that the ActRIIB-Fc:ALK3-Fc heterodimer has an altered binding profile/selectivity relative to either the ActRIIB-Fc homodimer or ALK3-Fe homodimer. The ActRIIB-Fc:ALK3-Fc heterodimer binds BMIP2 and BMP4 with exceptionally high affinity and displays greatly enhanced binding to BMIP5, BMIP6, BMIP7, GDF5, GDF6, and GDF7 compared with either homodimer. Compared to ActRIIB homodimer, the ActRIIB-Fc:ALK3-Fc heterodimer displays reduced binding to activin A, activin B, BMIP10, GDF8, and GDF11 and also discriminates among these ligands to a greater degree, particularly between activin A and activin B. In addition, the ability of ActRIIB-Fc homodimer to bind BMP9 and GDF3 with high affinity is absent for ActRIIB-Fc:ALK3-Fc heterodimer. See FIG. 7.

These results therefore demonstrate that the ActRIIB-Fc:ALK3-Fc heterodimer is a selective inhibitor of activin B, the GDF5/GDF6/GDF7 ligand subfamily, and several key BMP ligands excluding most notably BMIP9. Accordingly, an ActRIIB-Fc:ALK3-Fc heterodimer will be more useful than either an ActRIIB-Fc homodimer or an ALK3-Fc homodimer in certain applications where such selective antagonism is advantageous. Examples include therapeutic applications where it is desirable to retain antagonism of BMP2, BMP4, BMP5, and BMP6 or activin B but minimize antagonism of one or more ligands with anabolic muscle effects (e.g., activin A and GDF8) or ligands with angiogenic effects (e.g., BMP9 and BMP10).

### Example 6. Activity Profile of ActRIIB-Fc:ALK3-Fc Heterodimer in Mice Compared to ActRIIB-Fc Homodimer and ALK3-Fc Homodimer

Homodimeric and heterodimeric complexes were tested in mice to investigate differences in their activity profiles in vivo. Wild-type C₅₇BL/6 mice were dosed intraperitoneally with ActRIIB-Fc homodimer (10 mg/kg), ALK3-Fc homodimer (10 mg/kg), ActRIIB-Fc:ALK4-Fc heterodimer (3 or 10 mg/kg), or vehicle (phosphate-buffered saline, PBS) twice per week for 6.5 weeks (46 days) beginning at 10 weeks of age (n=5 mice per group). Study endpoints included body weight, total adipose mass as determined by nuclear magnetic resonance (NMR) at baseline and study completion (6.5 weeks), and total bone mineral density as determined by dual energy x-ray absorptiometry (DEXA) at baseline and 6.5 weeks.

Activity of ActRIIB-Fc and ALK3-Fc Complexes
in Wild-Type Mice Compared to Vehicle

| | ActRIIB-Fc | ALK3-Fc | ActRIIB-Fc:ALK3-Fc | |
|---|---|---|---|---|
| Endpoint | homodimer | homodimer | heterodimer | |
| 6.5 wk | 10 mg/kg | 10 mg/kg | 10 mg/kg | 3 mg/kg |
| Body weight | ↑ 23% * | ↓ 3% | ↓ 0.5% | ↓ 1% |
| Total adipose mass | ↓ 41% * | ↓ 12% | ↓ 14% * | ↓ 18% * |
| Total bone mineral | ↑ 8% * | ↑ 6% * | ↑ 9% * | ↑ 10% * |
| density | | | | |

* P < 0.05 vs. vehicle

Study results are summarized in the table above. As expected, the ActRIIB-Fc homodimer significantly increased body weight and total bone mineral density, and significantly reduced total adipose mass, all compared to vehicle. Also as expected, the ALK3-Fc homodimer significantly increased total bone mineral density compared to vehicle but unlike the ActRIIB-Fc homodimer did not significantly alter either body weight or total adipose mass. The ActRIIB-Fc:ALK3-Fc heterodimer notably displayed an activity profile different from either the ActRIIB-Fc homodimer or the ALK3-Fc homodimer. Treatment of mice with the ActRIIB-Fc:ALK4-Fc heterodimer at either dose level significantly increased bone mineral density at least as well either homodimer. However, unlike ALK3-Fc homodimer, the ActRIIB-Fc:ALK3-Fc heterodimer significantly reduced adipose mass, and unlike ActRIIB-Fc homodimer, the ActRIIB-Fc:ALK3-Fc heterodimer signficantly reduced adipose mass without altering body weight. Thus, an ActRIIB-Fc:ALK3-Fc heterodimer exerts beneficial effects on bone together with potentially beneficial effects on adipose tissue. This novel selectivity will be useful, for example, in treating patients in need of stimulatory effects on bone and inhibitory effects on fat but not in need of altered body weight.

### Example 7. Generation of an ActRIIB-Fc:ALK7-Fc Heterodimer

Applicants constructed a soluble ActRIIB-Fc:ALK7-Fc heteromeric complex comprising the extracellular domains of human ActRIIB and human ALK7, which are each fused to an Fc domain with a linker positioned between the extracellular domain and the Fc domain. The individual constructs are referred to as ActRIIB-Fc and ALK7-Fc, respectively.

Formation of heteromeric ALK7-Fc:ActRIIB-Fc may be guided by approaches similar to those described in Example 1.

In a first approach, the polypeptide sequence of the ActRIIB-Fc fusion protein and a nucleic acid sequence encoding it are provided above in Example 1 as SEQ ID NOs: 100-102.

The complementary ALK7-Fc fusion protein employs the TPA leader and is as follows (SEQ ID NO: 112):

| (SEQ ID NO: 112) | |
|---|---|
| 1 | MDAMKRGLCC VLLLCGAVFV SPGAGLKCVC LLCDSSNFTC QTEGACWASV |
| 51 | MLTNGKEQVI KSCVSLPELN AQVFCHSSNN VTKTECCFTD FCNNITLHLP |
| 101 | TASPNAPKLG PMETGGGTHT CPPCPAPELL GGPSVFLFPP KPKDTLMISR |
| 151 | TPEVTCVVVD VSHEDPEVKF NWYVDGVEVH NAKTKPREEQ YNSTYRVVSV |
| 201 | LTVLHQDWLN GKEYKCKVSN KALPAPIEKT ISKAKGQPRE PQVYTLPPSR |
| 251 | EEMTKNQVSL TCLVKGFYPS DIAVEWESNG QPENNYDTTP PVLDSDGSFF |
| 301 | LYSDLTVDKS RWQQGNVFSC SVMHEALHNH YTQKSLSLSP G |

The signal sequence and linker sequence are underlined. To promote formation of the ActRIIB-Fc:ALK7-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 112 may optionally be provided with a lysine added at the C-terminus.

This ALK7-Fc fusion protein is encoded by the following nucleic acid (SEQ ID NO: 113):

| (SEQ ID NO: 113) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGGACTGAA GTGTGTATGT CTTTTGTGTG |
| 101 | ATTCTTCAAA CTTTACCTGC CAAACAGAAG GAGCATGTTG GGCATCAGTC |
| 151 | ATGCTAACCA ATGGAAAAGA GCAGGTGATC AAATCCTGTG TCTCCCTTCC |
| 201 | AGAACTGAAT GCTCAAGTCT TCTGTCATAG TTCCAACAAT GTTACCAAAA |
| 251 | CCGAATGCTG CTTCACAGAT TTTTGCAACA ACATAACACT GCACCTTCCA |
| 301 | ACAGCATCAC CAAATGCCCC AAAACTTGGA CCCATGGAGA CCGGTGGTGG |
| 351 | AACTCACACA TGCCCACCGT GCCCAGCACC TGAACTCCTG GGGGGACCGT |
| 401 | CAGTCTTCCT CTTCCCCCCA AAACCCAAGG ACACCCTCAT GATCTCCCGG |
| 451 | ACCCCTGAGG TCACATGCGT GGTGGTGGAC GTGAGCCACG AAGACCCTGA |
| 501 | GGTCAAGTTC AACTGGTACG TGGACGGCGT GGAGGTGCAT AATGCCAAGA |
| 551 | CAAAGCCGCG GGAGGAGCAG TACAACAGCA CGTACCGTGT GGTCAGCGTC |
| 601 | CTCACCGTCC TGCACCAGGA CTGGCTGAAT GGCAAGGAGT ACAAGTGCAA |
| 651 | GGTCTCCAAC AAAGCCCTCC CAGCCCCCAT CGAGAAAACC ATCTCCAAAG |
| 701 | CCAAAGGGCA GCCCCGAGAA CCACAGGTGT ACACCCTGCC CCCATCCCGG |
| 751 | GAGGAGATGA CCAAGAACCA GGTCAGCCTG ACCTGCCTGG TCAAAGGCTT |
| 801 | CTATCCCAGC GACATCGCCG TGGAGTGGGA GAGCAATGGG CAGCCGGAGA |
| 851 | ACAACTACGA CACCACGCCT CCCGTGCTGG ACTCCGACGG CTCCTTCTTC |
| 901 | CTCTATAGCG ACCTCACCGT GGACAAGAGC AGGTGGCAGC AGGGGAACGT |
| 951 | CTTCTCATGC TCCGTGATGC ATGAGGCTCT GCACAACCAC TACACGCAGA |
| 1001 | AGAGCCTCTC CCTGTCTCCG GGT |

The mature ALK7-Fc fusion protein sequence (SEQ ID NO: 114) is expected to be as follows and may optionally be provided with a lysine added at the C-terminus.

| (SEQ ID NO: 114) | |
|---|---|
| 1 | GLKCVCLLCD SSNFTCQTEG ACWASVMLTN GKEQVIKSCV SLPELNAQVF |
| 51 | CHSSNNVTKT ECCFTDFCNN ITLHLPTASP NAPKLGPMET GGGTHTCPPC |
| 101 | PAPELLGGPS VFLFPPKPKD TLMISRTPEV TCVVVDVSHE DPEVKFNWYV |
| 151 | DGVEVHNAKT KPREEQYNST YRVVSVLTVL HQDWLNGKEY KCKVSNKALP |
| 201 | APIEKTISKA KGQPREPQVY TLPPSREEMT KNQVSLTCLV KGFYPSDIAV |
| 251 | EWESNGQPEN NYDTTPPVLD SDGSFFLYSD LTVDKSRWQQ GNVFSCSVMH |
| 301 | EALHNHYTQK SLSLSPG |

The ActRIIB-Fc and ALK7-Fc fusion proteins of SEQ ID NO: 102 and SEQ ID NO: 114, respectively, may be co-expressed and purified from a CHO cell line to give rise to a heteromeric complex comprising ActRIIB-Fc:ALK7-Fc.

In another approach to promoting the formation of heteromultimer complexes using asymmetric Fc fusion proteins, illustrated in the ActRIIB-Fc and ALK7-Fc polypeptide sequences of SEQ ID NOs: 401-402 and 405-406, respectively, the Fc domains are altered to introduce complementary hydrophobic interactions and an additional intermolecular disulfide bond. The ActRIIB-Fc fusion polypeptide sequences are discussed in Example 1.

The complementary form of ALK7-Fc fusion polypeptide (SEQ ID NO: 405) is as follows:

| (SEQ ID NO: 405) | |
|---|---|
| 1 | MDAMKRGLCC VLLLCGAVFV SPGAGLKCVC LLCDSSNFTC QTEGACWASV |
| 51 | MLTNGKEQVI KSCVSLPELN AQVFCHSSNN VTKTECCFTD FCNNITLHLP |
| 101 | TASPNAPKLG PMETGGGTHT CPPCPAPELL GGPSVFLFPP KPKDTLMISR |
| 151 | TPEVTCVVVD VSHEDPEVKF NWYVDGVEVH NAKTKPREEQ YNSTYRVVSV |
| 201 | LTVLHQDWLN GKEYKCKVSN KALPAPIEKT ISKAKGQPRE PQVCTLPPSR |
| 251 | EEMTKNQVSL SCAVKGFYPS DIAVEWESNG QPENNYKTTP PVLDSDGSFF |
| 301 | LVSKLTVDKS RWQQGNVFSC SVMHEALHNH YTQKSLSLSP GK |

The leader sequence and linker sequence are underlined. To guide heterodimer formation with the ActRIIB-Fc fusion polypeptide of SEQ ID NOs 401 and 402 above, four amino acid substitutions can be introduced into the Fc domain of the ALK7 fusion polypeptide as indicated by double underline above. Furthermore, the C-terminal lysine residue of the Fc domain can be deleted. The amino acid sequence of SEQ ID NO: 405 may optionally be provided with the lysine removed from the C-terminus.

The mature ALK7-Fc fusion protein sequence (SEQ ID NO: 406) is expected to be as follows and may optionally be provided with the lysine removed from the C-terminus.

| (SEQ ID NO: 406) | |
|---|---|
| 1 | GLKCVCLLCD SSNFTCQTEG ACWASVMLTN GKEQVIKSCV SLPELNAQVF |
| 51 | CHSSNNVTKT ECCFTDFCNN ITLHLPTASP NAPKLGPMET GGGTHTCPPC |
| 101 | PAPELLGGPS VFLFPPKPKD TLMISRTPEV TCVVVDVSHE DPEVKFNWYV |
| 151 | DGVEVHNAKT KPREEQYNST YRVVSVLTVL HQDWLNGKEY KCKVSNKALP |
| 201 | APIEKTISKA KGQPREPQVC TLPPSREEMT KNQVSLSCAV KGFYPSDIAV |
| 251 | EWESNGQPEN NYKTTPPVLD SDGSFFLVSK LTVDKSRWQQ GNVFSCSVMH |
| 301 | EALHNHYTQK SLSLSPGK |

The ActRIIB-Fc and ALK7-Fc proteins of SEQ ID NO: 402 and SEQ ID NO: 406, respectively, may be co-expressed and purified from a CHO cell line, to give rise to a heteromeric complex comprising ActRIIB-Fc:ALK7-Fc.

Purification of various ActRIIB-Fc:ALK7-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 8. Ligand Binding Profile of ActRIIB-Fc:ALK7-Fc Heterodimer Compared to ActRIIB-Fc Homodimer and ALK7-Fc Homodimer

A Biacore™-based binding assay was used to compare ligand binding selectivity of the ActRIIB-Fc:ALK7-Fc heterodimeric complex described above with that of ActRIIB-Fc and ALK7-Fc homodimeric complexes. The ActRIIB-Fc:ALK7-Fc heterodimer, ActRIIB-Fc homodimer, and ALK7-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand binding profile of ActRIIB-Fc:ALK7-Fc heterodimer
compared to ActRIIB-Fc homodimer and ALK7-Fc homodimer

| | ActRIIB-Fc | | | ALK7-FC | | | ActRIIB-Fc:ALK7-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |

| Activin A | 1.3 × 10⁷ | 1.4 × 10⁻⁴ | 11 | No binding | 4.4 × 10⁷ | 1.9 × 10⁻³ | 43 |
|---|---|---|---|---|---|---|---|
| Activin B | 1.5 × 10⁷ | 1.6 × 10⁻⁴ | 8 | No binding | 1.2 × 10⁷ | 2.0 × 10⁻⁴ | 17 |
| BMP5 | 2.6 × 10⁷ | 7.5 × 10⁻² | 2900 | No binding | 1.5 × 10⁵ | 8.5 × 10⁻³ | 57000 |
| BMP6 | 2.4 × 10⁷ | 3.9 × 10⁻³ | 160 | No binding | 1.2 × 10⁶ | 6.3 × 10⁻³ | 5300 |
| BMP9 | 1.2 × 10⁸ | 1.2 × 10⁻³ | 10 | No binding | Transient* | | >1400 |
| BMP10 | 5.9 × 10⁶ | 1.5 × 10⁻⁴ | 25 | No binding | 1.5 × 10⁷ | 2.8 × 10⁻³ | 190 |
| GDF3 | 1.4 × 10⁶ | 2.2 × 10⁻³ | 1500 | No binding | 2.3 × 10⁶ | 1.0 × 10⁻² | 4500 |
| GDF8 | 3.5 × 10⁶ | 2.4 × 10⁻⁴ | 69 | No binding | 3.7 × 10⁶ | 1.0 × 10⁻³ | 270 |
| GDF11 | 9.6 × 10⁷ | 1.5 × 10⁻⁴ | 2 | No binding | 9.5 × 10⁷ | 7.5 × 10⁻⁴ | 8 |

*Indeterminate due to transient nature of interaction

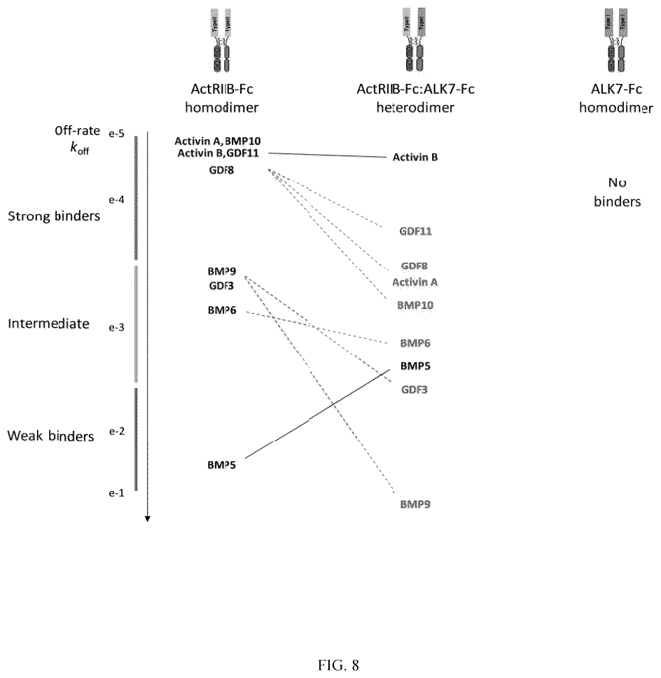

These comparative binding data demonstrate that the ActRIIB-Fc:ALK7-Fc heterodimer has a different binding profile compared to either the ActRIIB-Fc homodimer or ALK7-Fc homodimer. Interestingly, four of the five ligands with strong binding to ActRIIB-Fc homodimer (activin A, BMP10, GDF8, and GDF11) exhibit reduced binding to the ActRIIB-Fc:ALK7-Fc heterodimer, the exception being activin B which retains tight binding to the heterodimer. In addition, three ligands with intermediate binding to ActRIIB-Fc homodimer (GDF3, BMP6, and particularly BMP9) exhibit reduced binding to the ActRIIB-Fc:ALK7-Fc heterodimer. In contrast, BMP5 binds the ActRIIB-Fc:ALK7 heterodimer with intermediate strength despite only weak binding to ActRIIB-Fc homodimer. No ligands tested bind to ALK7-Fc homodimer. See FIG. 8.

These results therefore demonstrate that the ActRIIB-Fc:ALK7-Fc heterodimer is a more selective antagonist of activin B in comparison to a ActRIIB-Fc homodimer. Accordingly, an ActRIIB-Fc:ALK7-Fc heterodimer will be more useful than an ActRIIB-Fc homodimer in certain applications where such selective antagonism is advantageous. Examples include therapeutic applications where it is desirable to retain antagonism of activin B but minimize antagonism of one or more of activin A, GDF3, GDF8, GDF11, BMP9, or BMP10.

### Example 9. Generation of an ActRIIB-Fc:ALK2-Fc Heterodimer

Applicants constructed a soluble ActRIIB-Fc:ALK2-Fc heteromeric complex comprising the extracellular domains of human ActRIIB and human ALK2, which are each fused to an Fc domain with a linker positioned between the extracellular domain and the Fc domain. The individual constructs are referred to as ActRIIB-Fc and ALK2-Fc, respectively.

Formation of heteromeric ActRIIB-Fc:ALK2-Fc may be guided by approaches similar to those described in Example 1.

In a first approach, the polypeptide sequence of the ActRIIB-Fc fusion protein and a nucleic acid sequence encoding it are provided in Example 1 as SEQ ID NOs: 100-102.

The complementary ALK2-Fc fusion protein employs the TPA leader and is as follows (SEQ ID NO: 136):

| (SEQ ID NO: 136) | |
|---|---|
| 1 | MDAMKRGLCC VLLLCGAVFV SPGAMEDEKP KVNPKLYMCV CEGLSCGNED |
| 51 | HCEGQQCFSS LSINDGFHVY QKGCFQVYEQ GKMTCKTPPS PGQAVECCQG |
| 101 | DWCNRNITAQ LPTKGKSFPG TQNFHLETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVYTL PPSREEMTKN QVSLTCLVKG FYPSDIAVEW ESNGQPENNY |
| 301 | DTTPPVLDSD GSFFLYSDLT VDKSRWQQGN VFSCSVMHEA LHNHYTQKSL |
| 351 | SLSPG |

The signal sequence and linker sequence are underlined. To promote formation of the ActRIIB-Fc:ALK2-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 136 may optionally be provided with a lysine added at the C-terminus.

This ALK2-Fc fusion protein is encoded by the following nucleic acid (SEQ ID NO: 137):

| (SEQ ID NO: 137) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCATGGAAGA TGAGAAGCCC AAGGTCAACC |
| 101 | CCAAACTCTA CATGTGTGTG TGTGAAGGTC TCTCCTGCGG TAATGAGGAC |
| 151 | CACTGTGAAG GCCAGCAGTG CTTTTCCTCA CTGAGCATCA ACGATGGCTT |
| 201 | CCACGTCTAC CAGAAAGGCT GCTTCCAGGT TTATGAGCAG GGAAAGATGA |
| 251 | CCTGTAAGAC CCCGCCGTCC CCTGGCCAAG CTGTGGAGTG CTGCCAAGGG |
| 301 | GACTGGTGTA ACAGGAACAT CACGGCCCAG CTGCCCACTA AAGGAAAATC |
| 351 | CTTCCCTGGA ACACAGAATT TCCACTTGGA GACCGGTGGT GGAACTCACA |
| 401 | CATGCCCACC GTGCCCAGCA CCTGAACTCC TGGGGGGACC GTCAGTCTTC |
| 451 | CTCTTCCCCC CAAAACCCAA GGACACCCTC ATGATCTCCC GGACCCCTGA |
| 501 | GGTCACATGC GTGGTGGTGG ACGTGAGCCA CGAAGACCCT GAGGTCAAGT |
| 551 | TCAACTGGTA CGTGGACGGC GTGGAGGTGC ATAATGCCAA GACAAAGCCG |
| 601 | CGGGAGGAGC AGTACAACAG CACGTACCGT GTGGTCAGCG TCCTCACCGT |
| 651 | CCTGCACCAG GACTGGCTGA ATGGCAAGGA GTACAAGTGC AAGGTCTCCA |
| 701 | ACAAAGCCCT CCCAGCCCCC ATCGAGAAAA CCATCTCCAA AGCCAAAGGG |
| 751 | CAGCCCCGAG AACCACAGGT GTACACCCTG CCCCCATCCC GGGAGGAGAT |
| 801 | GACCAAGAAC CAGGTCAGCC TGACCTGCCT GGTCAAAGGC TTCTATCCCA |
| 851 | GCGACATCGC CGTGGAGTGG GAGAGCAATG GGCAGCCGGA GAACAACTAC |
| 901 | GACACCACGC CTCCCGTGCT GGACTCCGAC GGCTCCTTCT TCCTCTATAG |
| 951 | CGACCTCACC GTGGACAAGA GCAGGTGGCA GCAGGGGAAC GTCTTCTCAT |
| 1001 | GCTCCGTGAT GCATGAGGCT CTGCACAACC ACTACACGCA GAAGAGCCTC |
| 1051 | TCCCTGTCTC CGGGT |

The mature ALK2-Fc fusion protein sequence (SEQ ID NO: 138) is as follows and may optionally be provided with a lysine added at the C-terminus.

| (SEQ ID NO: 138) | |
|---|---|
| 1 | MEDEKPKVNP KLYMCVCEGL SCGNEDHCEG QQCFSSLSIN DGFHVYQKGC |
| 51 | FQVYEQGKMT CKTPPSPGQA VECCQGDWCN RNITAQLPTK GKSFPGTQNF |
| 101 | HLETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVYTLPPSR EEMTKNQVSL |
| 251 | TCLVKGFYPS DIAVEWESNG QPENNYDTTP PVLDSDGSFF LYSDLTVDKS |
| 301 | RWQQGNVFSC SVMHEALHNH YTQKSLSLSP G |

The ActRIIB-Fc and ALK2-Fc fusion proteins of SEQ ID NO: 102 and SEQ ID NO: 138, respectively, may be co-expressed and purified from a CHO cell line to give rise to a heteromeric complex comprising ActRIIB-Fc:ALK2-Fc.

In another approach to promoting the formation of heteromultimer complexes using asymmetric Fc fusion proteins, illustrated in the ActRIIB-Fc and ALK2-Fc polypeptide sequences of SEQ ID NOs: 401-402 and 421-422, respectively, the Fc domains are altered to introduce complementary hydrophobic interactions and an additional intermolecular disulfide bond. The ActRIIB-Fc fusion polypeptide sequences are discussed in Example 1.

The complementary form of ALK2-Fc fusion polypeptide (SEQ ID NO: 421) is as follows:

| (SEQ ID NO: 421) | |
|---|---|
| 1 | MDAMKRGLCC VLLLCGAVFV SPGAMEDEKP KVNPKLYMCV CEGLSCGNED |
| 51 | HCEGQQCFSS LSINDGFHVY QKGCFQVYEQ GKMTCKTPPS PGQAVECCQG |
| 101 | DWCNRNITAQ LPTKGKSFPG TQNFHLETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVCTL PPSREEMTKN QVSLSCAVKG FYPSDIAVEW ESNGQPENNY |
| 301 | KTTPPVLDSD GSFFLVSKLT VDKSRWQQGN VFSCSVMHEA LHNHYTQKSL |
| 351 | SLSPGK |

The leader sequence and linker sequence are underlined. To guide heterodimer formation with the ActRIIB-Fc fusion polypeptide of SEQ ID NOs 401 and 402 above, four amino acid substitutions can be introduced into the Fc domain of the ALK2 fusion polypeptide as indicated by double underline above. Furthermore, the C-terminal lysine residue of the Fc domain can be deleted. The amino acid sequence of SEQ ID NO: 421 may optionally be provided with the lysine removed from the C-terminus.

The mature ALK2-Fc fusion protein sequence (SEQ ID NO: 422) is as follows and may optionally be provided with the lysine removed from the C-terminus.

| (SEQ ID NO: 422) | |
|---|---|
| 1 | MEDEKPKVNP KLYMCVCEGL SCGNEDHCEG QQCFSSLSIN DGFHVYQKGC |
| 51 | FQVYEQGKMT CKTPPSPGQA VECCQGDWCN RNITAQLPTK GKSFPGTQNF |
| 101 | HLETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVCTLPPSR EEMTKNQVSL |
| 251 | SCAVKGFYPS DIAVEWESNG QPENNYKTTP PVLDSDGSFF LVSKLTVDKS |
| 301 | RWQQGNVFSC SVMHEALHNH YTQKSLSLSP GK |

The ActRIIB-Fc and ALK2-Fc proteins of SEQ ID NO: 402 and SEQ ID NO: 422, respectively, may be co-expressed and purified from a CHO cell line, to give rise to a heteromeric complex comprising ActRIIB-Fc:ALK2-Fc.

Purification of various ActRIIB-Fc:ALK2-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 10. Ligand Binding Profile of ActRIIB-Fc:ALK2-Fc Heterodimer Compared to ActRIIB-Fc Homodimer and ALK2-Fc Homodimer

A Biacore™-based binding assay was used to compare ligand binding selectivity of the ActRIIB-Fc:ALK2-Fc heterodimeric complex described above with that of ActRIIB-Fc and ALK2-Fc homodimeric complexes. The ActRIIB-Fc:ALK2-Fc heterodimer, ActRIIB-Fc homodimer, and ALK2-Fc homodimer were independently captured onto the system using an anti-Fe antibody. Ligands were injected and allowed to flow over the captured receptor protein. Results are summarized in the table below, in which ligand off-rates (k_{d}) most indicative of effective ligand traps are denoted in bold.

Ligand binding profile of ActRIIB-Fc:ALK2-Fc heterodimer
compared to ActRIIB-Fc homodimer and ALK2-Fc homodimer

| | ActRIIB-Fc | | | ALK2-Fc | | | ActRIIB-Fc:ALK2-Fc | | |
|---|---|---|---|---|---|---|---|---|---|
| | Homodimer | | | Homodimer | | | Heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |

| Activin A | 1.2 × 10⁷ | 1.7 × 10⁻⁴ | 15 | No binding | 3.4 × 10⁷ | 2.6 × 10⁻³ | 76 |
|---|---|---|---|---|---|---|---|
| Activin B | 3.8 × 10⁶ | 1.1 × 10⁻⁴ | 28 | No binding | 3.2 × 10⁶ | 1.5 × 10⁻⁴ | 47 |
| BMP5 | 3.8 × 10⁶ | 3.7 × 10⁻² | 9700 | No binding | 1.2 × 10⁶ | 1.4 × 10⁻³ | 1200 |
| BMP7 | 8.8 × 10⁶ | 1.4 × 10⁻² | 1600 | No binding | 1.5 × 10⁷ | 2.6 × 10⁻³ | 170 |
| BMP9 | 3.9 × 10⁷ | 1.3 × 10⁻³ | 34 | No binding | 3.2 × 10⁶ | 8.9 × 10⁻⁴ | 280 |
| BMP10 | 5.4 × 10⁷ | 2.8 × 10⁻⁴ | 5 | No binding | 5.5 × 10⁷ | 2.9 × 10⁻³ | 53 |
| GDF3 | 1.2 × 10⁶ | 2.0 × 10⁻³ | 1700 | No binding | 1.8 × 10⁶ | 1.2 × 10⁻² | 6500 |
| GDF5 | 1.2 × 10⁶ | 1.4 × 10⁻³ | 1100 | No binding | 8.8 × 10⁵ | 4.4 × 10⁻³ | 5000 |
| GDF6 | 1.5 × 10⁵ | 5.7 × 10⁻³ | 39000 | No binding | Transient* | | >240000 |
| GDF8 | 2.5 × 10⁶ | 3.2 × 10⁻⁴ | 130 | No binding | 2.1 × 10⁶ | 7.3 × 10⁻⁴ | 360 |
| GDF11 | 2.0 × 10⁶ | 2.2 × 10⁻⁴ | 110 | No binding | 1.6 × 10⁶ | 9.3 × 10⁻⁴ | 600 |

*Indeterminate due to transient nature of interaction

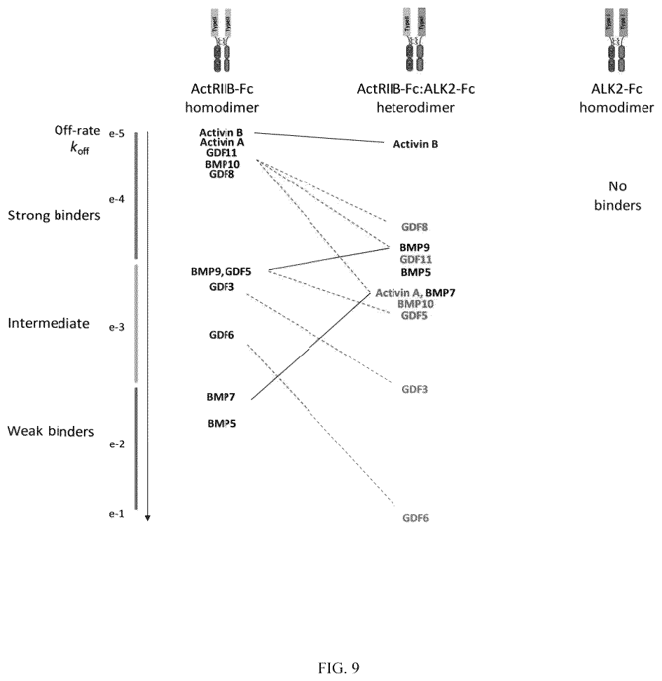

These comparative binding data demonstrate that the ActRIIB-Fc:ALK2-Fc heterodimer exhibits a ligand binding profile different from either the ActRIIB-Fc homodimer or the ALK2-Fc homodimer. ActRIIB-Fc:ALK2-Fc heterodimer exhibits preferential and strong binding to activin B, thus resembling ActRIIB-Fc:ALK7-Fc heterodimer (see Example 8). However, ActRIIB-Fc:ALK2-Fc heterodimer differs from ActRIIB-Fc:ALK7-Fc in part by retaining the tight binding to BMP9 characteristic of ActRIIB-Fc homodimer, whereas ActRIIB-Fc:ALK7-Fc binds BMP9 very weakly, if at all. No ligands tested bind to ALK2-Fc homodimer. See FIG. 9.

These results demonstrate that the ActRIIB-Fc:ALK2-Fc heterodimer is a more selective antagonist of activin B compared to ActRIIB-Fc homodimer. Accordingly, an ActRIIB-Fc:ALK2-Fc heterodimer will be useful in certain applications where such selective antagonism is advantageous. Examples include therapeutic applications where it is desirable to retain antagonism primarily of activin B and to supplement that with antagonism secondarily of BMP9, GDF8, and GDF11.

### Example 11. Generation of an ActRIIB-Fc:ALK5-Fc Heterodimer

Applicants constructed a soluble ActRIIB-Fc:ALK5-Fc heteromeric complex comprising the extracellular domains of human ActRIIB and human ALK5, which are each fused to an Fc domain with a linker positioned between the extracellular domain and the Fc domain. The individual constructs are referred to as ActRIIB-Fc and ALK5-Fc, respectively.

Formation of heteromeric ActRIIB-Fc: ALK5-Fc may be guided by approaches similar to those described in Example 1.

In a first approach, the polypeptide sequence of the ActRIIB-Fc fusion protein and a nucleic acid sequence encoding it are provided in Example 1 as SEQ ID NOs: 100-102.

The complementary ALK5-Fc fusion protein employs the TPA leader and is as follows (SEQ ID NO: 139):

| (SEQ ID NO: 139) | |
|---|---|
| 1 | MDAMKRGLCC VLLLCGAVFV SPGAALLPGA TALQCFCHLC TKDNFTCVTD |
| 51 | GLCFVSVTET TDKVIHNSMC IAEIDLIPRD RPFVCAPSSK TGSVTTTYCC |
| 101 | NQDHCNKIEL PTTVKSSPGL GPVETGGGTH TCPPCPAPEL LGGPSVFLFP |
| 151 | PKPKDTLMIS RTPEVTCVVV DVSHEDPEVK FNWYVDGVEV HNAKTKPREE |
| 201 | QYNSTYRVVS VLTVLHQDWL NGKEYKCKVS NKALPAPIEK TISKAKGQPR |
| 251 | EPQVYTLPPS REEMTKNQVS LTCLVKGFYP SDIAVEWESN GQPENNYDTT |
| 301 | PPVLDSDGSF FLYSDLTVDK SRWQQGNVFS CSVMHEALHN HYTQKSLSLS |
| 351 | PG |

The signal sequence and linker sequence are underlined. To promote formation of the ActRIIB-Fc:ALK5-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 139 may optionally be provided with a lysine added at the C-terminus.

This ALK5-Fc fusion protein is encoded by the following nucleic acid (SEQ ID NO: 140):

| (SEQ ID NO: 140) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGCGCTGCT CCCGGGGGCG ACGGCGTTAC |
| 101 | AGTGTTTCTG CCACCTCTGT ACAAAAGACA ATTTTACTTG TGTGACAGAT |
| 151 | GGGCTCTGCT TTGTCTCTGT CACAGAGACC ACAGACAAAG TTATACACAA |
| 201 | CAGCATGTGT ATAGCTGAAA TTGACTTAAT TCCTCGAGAT AGGCCGTTTG |
| 251 | TATGTGCACC CTCTTCAAAA ACTGGGTCTG TGACTACAAC ATATTGCTGC |
| 301 | AATCAGGACC ATTGCAATAA AATAGAACTT CCAACTACTG TAAAGTCATC |
| 351 | ACCTGGCCTT GGTCCTGTGG AAACCGGTGG TGGAACTCAC ACATGCCCAC |
| 401 | CGTGCCCAGC ACCTGAACTC CTGGGGGGAC CGTCAGTCTT CCTCTTCCCC |
| 451 | CCAAAACCCA AGGACACCCT CATGATCTCC CGGACCCCTG AGGTCACATG |
| 501 | CGTGGTGGTG GACGTGAGCC ACGAAGACCC TGAGGTCAAG TTCAACTGGT |
| 551 | ACGTGGACGG CGTGGAGGTG CATAATGCCA AGACAAAGCC GCGGGAGGAG |
| 601 | CAGTACAACA GCACGTACCG TGTGGTCAGC GTCCTCACCG TCCTGCACCA |
| 651 | GGACTGGCTG AATGGCAAGG AGTACAAGTG CAAGGTCTCC AACAAAGCCC |
| 701 | TCCCAGCCCC CATCGAGAAA ACCATCTCCA AAGCCAAAGG GCAGCCCCGA |
| 751 | GAACCACAGG TGTACACCCT GCCCCCATCC CGGGAGGAGA TGACCAAGAA |
| 801 | CCAGGTCAGC CTGACCTGCC TGGTCAAAGG CTTCTATCCC AGCGACATCG |
| 851 | CCGTGGAGTG GGAGAGCAAT GGGCAGCCGG AGAACAACTA CGACACCACG |
| 901 | CCTCCCGTGC TGGACTCCGA CGGCTCCTTC TTCCTCTATA GCGACCTCAC |
| 951 | CGTGGACAAG AGCAGGTGGC AGCAGGGGAA CGTCTTCTCA TGCTCCGTGA |
| 1001 | TGCATGAGGC TCTGCACAAC CACTACACGC AGAAGAGCCT CTCCCTGTCT |
| 1051 | CCGGGT |

The mature ALK5-Fc fusion protein sequence (SEQ ID NO: 141) is as follows and may optionally be provided with a lysine added at the C-terminus.

| (SEQ ID NO: 141) | |
|---|---|
| 1 | ALLPGATALQ CFCHLCTKDN FTCVTDGLCF VSVTETTDKV IHNSMCIAEI |
| 51 | DLIPRDRPFV CAPSSKTGSV TTTYCCNQDH CNKIELPTTV KSSPGLGPVE |
| 101 | TGGGTHTCPP CPAPELLGGP SVFLFPPKPK DTLMISRTPE VTCVVVDVSH |
| 151 | EDPEVKFNWY VDGVEVHNAK TKPREEQYNS TYRVVSVLTV LHQDWLNGKE |
| 201 | YKCKVSNKAL PAPIEKTISK AKGQPREPQV YTLPPSREEM TKNQVSLTCL |
| 251 | VKGFYPSDIA VEWESNGQPE NNYDTTPPVL DSDGSFFLYS DLTVDKSRWQ |
| 301 | QGNVFSCSVM HEALHNHYTQ KSLSLSPG |

The ActRIIB-Fc and ALK5-Fc fusion proteins of SEQ ID NO: 102 and SEQ ID NO: 141,G-respectively, may be co-expressed and purified from a CHO cell line to give rise to a heteromeric complex comprising ActRIIB-Fc:ALK5-Fc.

In another approach to promoting the formation of heteromultimer complexes using asymmetric Fc fusion proteins, illustrated in the ActRIIB-Fc and ALK5-Fc polypeptide sequences of SEQ ID NOs: 401-402 and 423-424, respectively, the Fc domains are altered to introduce complementary hydrophobic interactions and an additional intermolecular disulfide bond. The ActRIIB-Fc fusion polypeptide sequences are discussed in Example 1.

The complementary form of ALK5-Fc fusion polypeptide (SEQ ID NO: 423) is as follows:

| (SEQ ID NO: 423) | |
|---|---|
| 1 | MDAMKRGLCC VLLLCGAVFV SPGAALLPGA TALQCFCHLC TKDNFTCVTD |
| 51 | GLCFVSVTET TDKVIHNSMC IAEIDLIPRD RPFVCAPSSK TGSVTTTYCC |
| 101 | NQDHCNKIEL PTTVKSSPGL GPVETGGGTH TCPPCPAPEL LGGPSVFLFP |
| 151 | PKPKDTLMIS RTPEVTCVVV DVSHEDPEVK FNWYVDGVEV HNAKTKPREE |
| 201 | QYNSTYRVVS VLTVLHQDWL NGKEYKCKVS NKALPAPIEK TISKAKGQPR |
| 251 | EPQVCTLPPS REEMTKNQVS LSCAVKGFYP SDIAVEWESN GQPENNYKTT |
| 301 | PPVLDSDGSF FLVSKLTVDK SRWQQGNVFS CSVMHEALHN HYTQKSLSLS |
| 351 | PGK |

The leader sequence and linker sequence are underlined. To guide heterodimer formation with the ActRIIB-Fc fusion polypeptide of SEQ ID NOs 401 and 402 above, four amino acid substitutions can be introduced into the Fc domain of the ALK5 fusion polypeptide as indicated by double underline above. Furthermore, the C-terminal lysine residue of the Fe domain can be deleted. The amino acid sequence of SEQ ID NO: 423 may optionally be provided with the lysine removed from the C-terminus.

The mature ALK5-Fc fusion protein sequence (SEQ ID NO: 424) is as follows and may optionally be provided with the lysine removed from the C-terminus.

| (SEQ ID NO: 424) | |
|---|---|
| 1 | ALLPGATALQ CFCHLCTKDN FTCVTDGLCF VSVTETTDKV IHNSMCIAEI |
| 51 | DLIPRDRPFV CAPSSKTGSV TTTYCCNQDH CNKIELPTTV KSSPGLGPVE |
| 101 | TGGGTHTCPP CPAPELLGGP SVFLFPPKPK DTLMISRTPE VTCVVVDVSH |
| 151 | EDPEVKFNWY VDGVEVHNAK TKPREEQYNS TYRVVSVLTV LHQDWLNGKE |
| 201 | YKCKVSNKAL PAPIEKTISK AKGQPREPQV CTLPPSREEM TKNQVSLSCA |
| 251 | VKGFYPSDIA VEWESNGQPE NNYKTTPPVL DSDGSFFLVS KLTVDKSRWQ |
| 301 | QGNVFSCSVM HEALHNHYTQ KSLSLSPGK |

The ActRIIB-Fc and ALK5-Fc proteins of SEQ ID NO: 402 and SEQ ID NO: 424, respectively, may be co-expressed and purified from a CHO cell line, to give rise to a heteromeric complex comprising ActRIITB-Fc:ALK5-Fc.

Purification of various ActRIIB-Fc:ALK5-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 12. Ligand Binding Profile of ActRIIB-Fc:ALK5-Fc Heterodimer Compared to ActRIIB-Fc Homodimer and ALK5-Fc Homodimer

A Biacore™-based binding assay was used to compare ligand binding selectivity of the ActRIIB-Fc:ALK5-Fc heterodimeric complex described above with that of ActRIIB-Fc and ALK5-Fc homodimeric complexes. The ActRIIB-Fc:ALK5-Fc heterodimer, ActRIIB-Fc homodimer, and ALK5-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand binding profile of ActRIIB-Fc:ALK5-Fc heterodimer
compared to ActRIIB-Fc homodimer and ALK5-Fc homodimer

| | ActRIIB-Fc | | | ALK5-Fc | | | ActRIIB-Fc:ALK5-Fc | | |
|---|---|---|---|---|---|---|---|---|---|
| | Homodimer | | | Homodimer | | | Heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |

| Activin A | 1.2 × 10⁷ | 2.3 × 10⁻⁴ | 19 | No binding | 3.6 × 10⁷ | 1.6 × 10⁻³ | 46 |
|---|---|---|---|---|---|---|---|
| Activin B | 5.1 × 10⁶ | 1.0 × 10⁻⁴ | 20 | No binding | 3.9 × 10⁶ | 3.1 × 10⁻⁴ | 79 |
| BMP6 | 6.4 × 10⁶ | 7.0 × 10⁻³ | 1100 | No binding | 9.3 × 10⁶ | 1.5 × 10⁻² | 1700 |
| BMP9 | 3.9 × 10⁷ | 1.3 × 10⁻³ | 34 | No binding | Transient* | | >6600 |
| BMP10 | 2.1 × 10⁷ | 3.8 × 10⁻⁴ | 18 | No binding | 2.3 × 10⁷ | 2.2 × 10⁻³ | 150 |
| GDF3 | 4.7 × 10⁵ | 1.8 × 10⁻³ | 3900 | No binding | 1.1 × 10⁵ | 9.7 × 10⁻³ | 8500 |
| GDF8 | 1.2 × 10⁶ | 1.9 × 10⁻⁴ | 160 | No binding | 1.1 × 10⁶ | 5.2 × 10⁻⁴ | 490 |
| GDF11 | 1.9 × 10⁶ | 1.4 × 10⁻⁴ | 74 | No binding | 2.3 × 10⁶ | 4.6 × 10⁻⁴ | 600 |

*Indeterminate due to transient nature of interaction

### Example 13. Generation of an ActRIIB-Fc:ALK6-Fc Heterodimer

A soluble ActRIIB-Fc:ALK6-Fc heteromeric complex can be generated comprising the extracellular domains of human ActRIIB and human ALK6, which can each be fused to an Fc domain with a linker positioned between the extracellular domain and the Fc domain. The individual constructs are referred to as ActRIIB-Fc and ALK6-Fc, respectively.

Formation of heteromeric ActRIIB-Fc: ALK6-Fc may be guided by approaches similar to those described in Example 1.

In a first approach, the polypeptide sequence of the ActRIIB-Fc fusion protein and a nucleic acid sequence encoding it are provided above in Example 1 as SEQ ID NOs: 100-102.

The complementary ALK6-Fc fusion protein employs the TPA leader and is as follows (SEQ ID NO: 142):

| (SEQ ID NO: 142) | |
|---|---|
| 1 | MDAMKRGLCC VLLLCGAVFV SPGAKKEDGE STAPTPRPKV LRCKCHHHCP |
| 51 | EDSVNNICST DGYCFTMIEE DDSGLPVVTS GCLGLEGSDF QCRDTPIPHQ |
| 101 | RRSIECCTER NECNKDLHPT LPPLKNRDFV DGPIHHRTGG GTHTCPPCPA |
| 151 | PELLGGPSVF LFPPKPKDTL MISRTPEVTC VVVDVSHEDP EVKFNWYVDG |
| 201 | VEVHNAKTKP REEQYNSTYR VVSVLTVLHQ DWLNGKEYKC KVSNKALPAP |
| 251 | IEKTISKAKG QPREPQVYTL PPSREEMTKN QVSLTCLVKG FYPSDIAVEW |
| 301 | ESNGQPENNY DTTPPVLDSD GSFFLYSDLT VDKSRWQQGN VFSCSVMHEA |
| 351 | LHNHYTQKSL SLSPG |

The signal sequence and linker sequence are underlined. To promote formation of the ActRIIB-Fc:ALK6-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 142 may optionally be provided with a lysine added at the C-terminus.

This ALK6-Fc fusion protein is encoded by the following nucleic acid (SEQ ID NO: 143):

| (SEQ ID NO: 143) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCAAGAAAGA GGATGGTGAG AGTACAGCCC |
| 101 | CCACCCCCCG TCCAAAGGTC TTGCGTTGTA AATGCCACCA CCATTGTCCA |
| 151 | GAAGACTCAG TCAACAATAT TTGCAGCACA GACGGATATT GTTTCACGAT |
| 201 | GATAGAAGAG GATGACTCTG GGTTGCCTGT GGTCACTTCT GGTTGCCTAG |
| 251 | GACTAGAAGG CTCAGATTTT CAGTGTCGGG ACACTCCCAT TCCTCATCAA |
| 301 | AGAAGATCAA TTGAATGCTG CACAGAAAGG AACGAATGTA ATAAAGACCT |
| 351 | ACACCCTACA CTGCCTCCAT TGAAAAACAG AGATTTTGTT GATGGACCTA |
| 401 | TACACCACAG GACCGGTGGT GGAACTCACA CATGCCCACC GTGCCCAGCA |
| 451 | CCTGAACTCC TGGGGGGACC GTCAGTCTTC CTCTTCCCCC CAAAACCCAA |
| 501 | GGACACCCTC ATGATCTCCC GGACCCCTGA GGTCACATGC GTGGTGGTGG |
| 551 | ACGTGAGCCA CGAAGACCCT GAGGTCAAGT TCAACTGGTA CGTGGACGGC |
| 601 | GTGGAGGTGC ATAATGCCAA GACAAAGCCG CGGGAGGAGC AGTACAACAG |
| 651 | CACGTACCGT GTGGTCAGCG TCCTCACCGT CCTGCACCAG GACTGGCTGA |
| 701 | ATGGCAAGGA GTACAAGTGC AAGGTCTCCA ACAAAGCCCT CCCAGCCCCC |
| 751 | ATCGAGAAAA CCATCTCCAA AGCCAAAGGG CAGCCCCGAG AACCACAGGT |
| 801 | GTACACCCTG CCCCCATCCC GGGAGGAGAT GACCAAGAAC CAGGTCAGCC |
| 851 | TGACCTGCCT GGTCAAAGGC TTCTATCCCA GCGACATCGC CGTGGAGTGG |
| 901 | GAGAGCAATG GGCAGCCGGA GAACAACTAC GACACCACGC CTCCCGTGCT |
| 951 | GGACTCCGAC GGCTCCTTCT TCCTCTATAG CGACCTCACC GTGGACAAGA |
| 1001 | GCAGGTGGCA GCAGGGGAAC GTCTTCTCAT GCTCCGTGAT GCATGAGGCT |
| 1051 | CTGCACAACC ACTACACGCA GAAGAGCCTC TCCCTGTCTC CGGGT |

The mature ALK6-Fc fusion protein sequence (SEQ ID NO: 144) is as follows and may optionally be provided with a lysine added at the C-terminus.

| (SEQ ID NO: 144) | |
|---|---|
| 1 | KKEDGESTAP TPRPKVLRCK CHHHCPEDSV NNICSTDGYC FTMIEEDDSG |
| 51 | LPVVTSGCLG LEGSDFQCRD TPIPHQRRSI ECCTERNECN KDLHPTLPPL |
| 101 | KNRDFVDGPI HHRTGGGTHT CPPCPAPELL GGPSVFLFPP KPKDTLMISR |
| 151 | TPEVTCVVVD VSHEDPEVKF NWYVDGVEVH NAKTKPREEQ YNSTYRVVSV |
| 201 | LTVLHQDWLN GKEYKCKVSN KALPAPIEKT ISKAKGQPRE PQVYTLPPSR |
| 251 | EEMTKNQVSL TCLVKGFYPS DIAVEWESNG QPENNYDTTP PVLDSDGSFF |
| 301 | LYSDLTVDKS RWQQGNVFSC SVMHEALHNH YTQKSLSLSP G |

The ActRIIB-Fc and ALK6-Fc fusion proteins of SEQ ID NO: 102 and SEQ ID NO: 144, respectively, may be co-expressed and purified from a CHO cell line to give rise to a heteromeric complex comprising ActRIIB-Fc:ALK6-Fc.

In another approach to promoting the formation of heteromultimer complexes using asymmetric Fc fusion proteins, illustrated in the ActRIIB-Fc and ALK6-Fc polypeptide sequences of SEQ ID NOs: 401-402 and 425-426, respectively, the Fc domains can be altered to introduce complementary hydrophobic interactions and an additional intermolecular disulfide bond. The ActRIIB-Fc fusion polypeptide sequences are discussed in Example 1.

The complementary form of ALK6-Fc fusion polypeptide (SEQ ID NO: 425) is as follows:

| (SEQ ID NO: 425) | |
|---|---|
| 1 | MDAMKRGLCC VLLLCGAVFV SPGAKKEDGE STAPTPRPKV LRCKCHHHCP |
| 51 | EDSVNNICST DGYCFTMIEE DDSGLPVVTS GCLGLEGSDF QCRDTPIPHQ |
| 101 | RRSIECCTER NECNKDLHPT LPPLKNRDFV DGPIHHRTGG GTHTCPPCPA |
| 151 | PELLGGPSVF LFPPKPKDTL MISRTPEVTC VVVDVSHEDP EVKFNWYVDG |
| 201 | VEVHNAKTKP REEQYNSTYR VVSVLTVLHQ DWLNGKEYKC KVSNKALPAP |
| 251 | IEKTISKAKG QPREPQVCTL PPSREEMTKN QVSLSCAVKG FYPSDIAVEW |
| 301 | ESNGQPENNY KTTPPVLDSD GSFFLVSKLT VDKSRWQQGN VFSCSVMHEA |
| 351 | LHNHYTQKSL SLSPGK |

The leader sequence and linker sequence are underlined. To guide heterodimer formation with the ActRIIB-Fc fusion polypeptide of SEQ ID NOs 401 and 402 above, four amino acid substitutions can be introduced into the Fc domain of the ALK6 fusion polypeptide as indicated by double underline above. Furthermore, the C-terminal lysine residue of the Fc domain can be deleted. The amino acid sequence of SEQ ID NO: 425 may optionally be provided with the lysine removed from the C-terminus.

The mature ALK6-Fc fusion protein sequence (SEQ ID NO: 426) can be as follows and may optionally be provided with the lysine removed from the C-terminus.

| (SEQ ID NO: 426) | |
|---|---|
| 1 | KKEDGESTAP TPRPKVLRCK CHHHCPEDSV NNICSTDGYC FTMIEEDDSG |
| 51 | LPVVTSGCLG LEGSDFQCRD TPIPHQRRSI ECCTERNECN KDLHPTLPPL |
| 101 | KNRDFVDGPI HHRTGGGTHT CPPCPAPELL GGPSVFLFPP KPKDTLMISR |
| 151 | TPEVTCVVVD VSHEDPEVKF NWYVDGVEVH NAKTKPREEQ YNSTYRVVSV |
| 201 | LTVLHQDWLN GKEYKCKVSN KALPAPIEKT ISKAKGQPRE PQVCTLPPSR |
| 251 | EEMTKNQVSL SCAVKGFYPS DIAVEWESNG QPENNYKTTP PVLDSDGSFF |
| 301 | LVSKLTVDKS RWQQGNVFSC SVMHEALHNH YTQKSLSLSP GK |

The ActRIIB-Fc and ALK6-Fc proteins of SEQ ID NO: 402 and SEQ ID NO: 426, respectively, may be co-expressed and purified from a CHO cell line, to give rise to a heteromeric complex comprising ActRIIB-Fc:ALK6-Fc.

Purification of various ActRIIB-Fc:ALK6-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 14. Generation of an ActRIIA-Fc:ALK4-Fc Heterodimer

Applicants constructed a soluble ActRIIA-Fc:ALK4-Fc heteromeric complex comprising the extracellular domains of human ActRIIA and human ALK4, which are each separately fused to an Fc domain with a linker positioned between the extracellular domain and the Fc domain. The individual constructs are referred to as ActRIIA-Fc fusion polypeptide and ALK4-Fc fusion polypeptide, respectively.

Formation of heteromeric ActRIIA-Fc:ALK4-Fc may be guided by approaches similar to those described in Example 1. In a first approach, one Fc domain is altered to introduce cationic amino acids at the interaction face, while the other Fc domain is altered to introduce anionic amino acids at the interaction face.

The ActRIIA-Fc polypeptide sequence (SEQ ID NO: 118) is shown below:

| (SEQ ID NO: 118) | |
|---|---|
| 1 | MDAMKRGLCC VLLLCGAVFV SPGAAILGRS ETQECLFFNA NWEKDRTNQT |
| 51 | GVEPCYGDKD KRRHCFATWK NISGSIEIVK QGCWLDDINC YDRTDCVEKK |
| 101 | DSPEVYFCCC EGNMCNEKFS YFPEMEVTQP TSNPVTPKPP TGGGTHTCPP |
| 151 | CPAPELLGGP SVFLFPPKPK DTLMISRTPE VTCVVVDVSH EDPEVKFNWY |
| 201 | VDGVEVHNAK TKPREEQYNS TYRVVSVLTV LHQDWLNGKE YKCKVSNKAL |
| 251 | PAPIEKTISK AKGQPREPQV YTLPPSRKEM TKNQVSLTCL VKGFYPSDIA |
| 301 | VEWESNGQPE NNYKTTPPVL KSDGSFFLYS KLTVDKSRWQ QGNVFSCSVM |
| 351 | HEALHNHYTQ KSLSLSPGK |

The leader sequence and linker sequence are underlined. To promote formation of the ActRIIA-Fc:ALK4-Fc heterodimer rather than either of the possible homodimeric complexes, two amino acid substitutions (replacing acidic amino acids with lysine) can be introduced into the Fe domain of the ActRIIA fusion protein as indicated by double underline above. The amino acid sequence of SEQ ID NO: 118 may optionally be provided with the lysine removed from the C-terminus.

This ActRIIA-Fc fusion protein is encoded by the following nucleic acid sequence (SEQ ID NO: 119):

| (SEQ ID NO: 119) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGCTATACT TGGTAGATCA GAAACTCAGG |
| 101 | AGTGTCTTTT CTTTAATGCT AATTGGGAAA AAGACAGAAC CAATCAAACT |
| 151 | GGTGTTGAAC CGTGTTATGG TGACAAAGAT AAACGGCGGC ATTGTTTTGC |
| 201 | TACCTGGAAG AATATTTCTG GTTCCATTGA AATAGTGAAA CAAGGTTGTT |
| 251 | GGCTGGATGA TATCAACTGC TATGACAGGA CTGATTGTGT AGAAAAAAAA |
| 301 | GACAGCCCTG AAGTATATTT CTGTTGCTGT GAGGGCAATA TGTGTAATGA |
| 351 | AAAGTTTTCT TATTTTCCGG AGATGGAAGT CACACAGCCC ACTTCAAATC |
| 401 | CAGTTACACC TAAGCCACCC ACCGGTGGTG GAACTCACAC ATGCCCACCG |
| 451 | TGCCCAGCAC CTGAACTCCT GGGGGGACCG TCAGTCTTCC TCTTCCCCCC |
| 501 | AAAACCCAAG GACACCCTCA TGATCTCCCG GACCCCTGAG GTCACATGCG |
| 551 | TGGTGGTGGA CGTGAGCCAC GAAGACCCTG AGGTCAAGTT CAACTGGTAC |
| 601 | GTGGACGGCG TGGAGGTGCA TAATGCCAAG ACAAAGCCGC GGGAGGAGCA |
| 651 | GTACAACAGC ACGTACCGTG TGGTCAGCGT CCTCACCGTC CTGCACCAGG |
| 701 | ACTGGCTGAA TGGCAAGGAG TACAAGTGCA AGGTCTCCAA CAAAGCCCTC |
| 751 | CCAGCCCCCA TCGAGAAAAC CATCTCCAAA GCCAAAGGGC AGCCCCGAGA |
| 801 | ACCACAGGTG TACACCCTGC CCCCATCCCG GAAGGAGATG ACCAAGAACC |
| 851 | AGGTCAGCCT GACCTGCCTG GTCAAAGGCT TCTATCCCAG CGACATCGCC |
| 901 | GTGGAGTGGG AGAGCAATGG GCAGCCGGAG AACAACTACA AGACCACGCC |
| 951 | TCCCGTGCTG AAGTCCGACG GCTCCTTCTT CCTCTATAGC AAGCTCACCG |
| 1001 | TGGACAAGAG CAGGTGGCAG CAGGGGAACG TCTTCTCATG CTCCGTGATG |
| 1051 | CATGAGGCTC TGCACAACCA CTACACGCAG AAGAGCCTCT CCCTGTCTCC |
| 1101 | GGGTAAA |

The mature ActRIIA-Fc fusion polypeptide (SEQ ID NO: 120) is as follows and may optionally be provided with the lysine removed from the C-terminus.

| (SEQ ID NO: 120) | |
|---|---|
| 1 | ILGRSETQEC LFFNANWEKD RTNQTGVEPC YGDKDKRRHC FATWKNISGS |
| 51 | IEIVKQGCWL DDINCYDRTD CVEKKDSPEV YFCCCEGNMC NEKFSYFPEM |
| 101 | EVTQPTSNPV TPKPPTGGGT HTCPPCPAPE LLGGPSVFLF PPKPKDTLMI |
| 151 | SRTPEVTCVV VDVSHEDPEV KFNWYVDGVE VHNAKTKPRE EQYNSTYRVV |
| 201 | SVLTVLHQDW LNGKEYKCKV SNKALPAPIE KTISKAKGQP REPQVYTLPP |
| 251 | SRKEMTKNQV SLTCLVKGFY PSDIAVEWES NGQPENNYKT TPPVLKSDGS |
| 301 | FFLYSKLTVD KSRWQQGNVF SCSVMHEALH NHYTQKSLSL SPGK |

In this first approach, the polypeptide sequence of the complementary ALK4-Fc fusion protein and a nucleic acid sequence encoding it are provided above in Example 1 as SEQ ID NOs: 104-106.

The ActRIIA-Fc and ALK4-Fc proteins of SEQ ID NO: 120 and SEQ ID NO: 106, respectively, may be co-expressed and purified from a CHO cell line, to give rise to a heteromeric complex comprising ActRIIA-Fc:ALK4-Fc.

In a second approach to promote the formation of heteromultimer complexes using asymmetric Fc fusion proteins, the Fc domains are altered to introduce complementary hydrophobic interactions and an additional intermolecular disulfide bond.

The ActRIIA-Fc polypeptide sequence (SEQ ID NO: 409) is shown below:

| (SEQ ID NO: 409) | |
|---|---|
| 1 | MDAMKRGLCC VLLLCGAVFV SPGAAILGRS ETQECLFFNA NWEKDRTNQT |
| 51 | GVEPCYGDKD KRRHCFATWK NISGSIEIVK QGCWLDDINC YDRTDCVEKK |
| 101 | DSPEVYFCCC EGNMCNEKFS YFPEMEVTQP TSNPVTPKPP TGGGTHTCPP |
| 151 | CPAPELLGGP SVFLFPPKPK DTLMISRTPE VTCVVVDVSH EDPEVKFNWY |
| 201 | VDGVEVHNAK TKPREEQYNS TYRVVSVLTV LHQDWLNGKE YKCKVSNKAL |
| 251 | PAPIEKTISK AKGQPREPQV YTLPPCREEM TKNQVSLWCL VKGFYPSDIA |
| 301 | VEWESNGQPE NNYKTTPPVL DSDGSFFLYS KLTVDKSRWQ QGNVFSCSVM |
| 351 | HEALHNHYTQ KSLSLSPGK |

The leader sequence and linker sequence are underlined. To promote formation of the ActRIIA-Fc:ALK4-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 409 may optionally be provided with the lysine removed from the C-terminus.

The mature ActRIIA-Fc fusion polypeptide (SEQ ID NO: 410) is as follows and may optionally be provided with the lysine removed from the C-terminus.

| (SEQ ID NO: 410) | |
|---|---|
| 1 | ILGRSETQEC LFFNANWEKD RTNQTGVEPC YGDKDKRRHC FATWKNISGS |
| 51 | IEIVKQGCWL DDINCYDRTD CVEKKDSPEV YFCCCEGNMC NEKFSYFPEM |
| 101 | EVTQPTSNPV TPKPPTGGGT HTCPPCPAPE LLGGPSVFLF PPKPKDTLMI |
| 151 | SRTPEVTCVV VDVSHEDPEV KFNWYVDGVE VHNAKTKPRE EQYNSTYRVV |
| 201 | SVLTVLHQDW LNGKEYKCKV SNKALPAPIE KTISKAKGQP REPQVYTLPP |
| 251 | CREEMTKNQV SLWCLVKGFY PSDIAVEWES NGQPENNYKT TPPVLDSDGS |
| 301 | FFLYSKLTVD KSRWQQGNVF SCSVMHEALH NHYTQKSLSL SPGK |

In this second approach, the polypeptide sequence of the complementary ALK4-Fc fusion protein and a nucleic acid sequence encoding it are provided above in Example 1 as SEQ ID NOs: 403-404.

The ActRIIA-Fc and ALK4-Fc proteins of SEQ ID NO: 410 and SEQ ID NO: 404, respectively, may be co-expressed and purified from a CHO cell line, to give rise to a heteromeric complex comprising ActRIIA-Fc:ALK4-Fc.

Purification of various ActRIIA-Fc:ALK4-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 15. Ligand Binding Profile of ActRIIA-Fc:ALK4-Fc Heterodimer Compared to ActRIIA-Fc Homodimer and ALK4-Fc Homodimer

A Biacore™-based binding assay was used to compare ligand binding selectivity of the ActRIIA-Fc:ALK4-Fc heterodimeric complex described above with that of ActRIIA-Fc and ALK4-Fc homodimeric complexes. The ActRIA-Fc:ALK4-Fc heterodimer, ActRIIA-Fc homodimer, and ALK4-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand binding profile of ActRIIA-Fc:ALK4-Fc heterodimer
compared to ActRIIA-Fc homodimer and ALK4-Fc homodimer

| | ActRIIA-Fc | | | ALK4-Fc | | | ActRIIA-Fc:ALK4-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |
| Activin A | 1.4 × 10⁷ | 6.2 × 10⁻⁴ | 45 | 5.8 × 10⁵ | 1.2 × 10⁻² | 20000 | 7.4 × 10⁶ | 2.4 × 10⁻⁴ | 32 |
| Activin B | 1.1 × 10⁷ | 1.1 × 10⁻⁴ | 10 | No binding | | | 9.5 × 10⁶ | 4.8 × 10⁻⁴ | 50 |
| Activin AB | 2.8 × 10⁷ | 2.6 × 10⁻⁴ | 9 | 1.8 × 10⁶ | 3.6 × 10⁻³ | 2000 | 1.8 × 10⁷ | 2.3 × 10⁻⁴ | 13 |
| Activin AC | 2.2 × 10⁷ | 7.9 × 10⁻³ | 360 | No binding | | | 3.2 × 10⁶ | 5.4 × 10⁻⁴ | 170 |
| BMP6 | 2.7 × 10⁸ | 2.2 × 10⁻² | 800 | No binding | | | 5.4 × 10⁶ | 1.2 × 10⁻² | 2200 |
| BMP7 | 8.9 × 10⁶ | 3.3 × 10⁻² | 3700 | No binding | | | 2.0 × 10⁷ | 7.2 × 10⁻² | 3500 |
| BMP9 | Transient* | | >10000 | — | | | No binding | | |
| BMP10 | 2.9 × 10⁷ | 2.5 × 10⁻³ | 85 | No binding | | | Transient* | | >6000 |
| GDF3 | 1.5 × 10⁶ | 3.6 × 10⁻³ | 2400 | — | | | 4.9 × 10⁷ | 4.8 × 10⁻³ | 9800 |
| GDF8 | 1.4 × 10⁶ | 1.4 × 10⁻³ | 99 | 1.3 × 10⁵ | 1.9 × 10⁻³ | 15000† | 1.8 × 10⁷ | 2.8 × 10⁻³ | 150 |
| GDF11 | 7.3 × 10⁷ | 9.2 × 10⁻⁴ | 13 | 5.0 × 10⁶ | 4.8 × 10⁻³ | 970† | 3.0 × 10⁷ | 6.5 × 10⁻⁴ | 22 |

*Indeterminate due to transient nature of interaction
†Very low signal
— Not tested

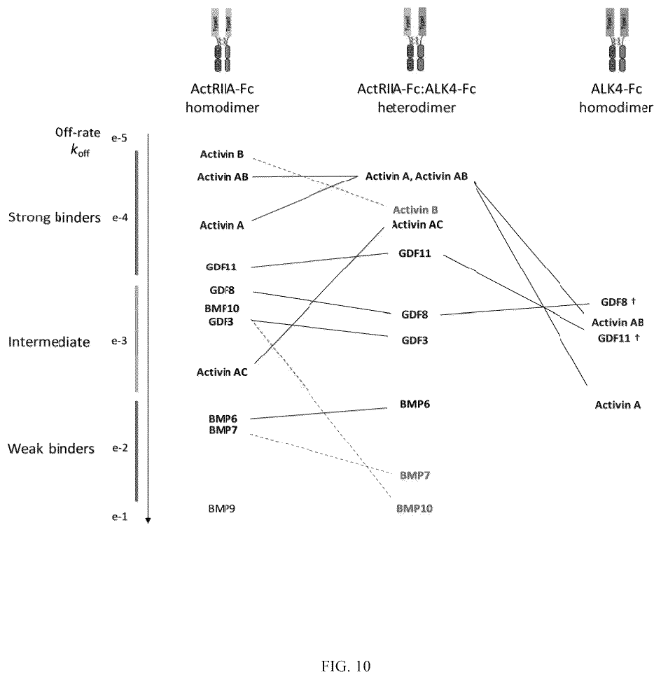

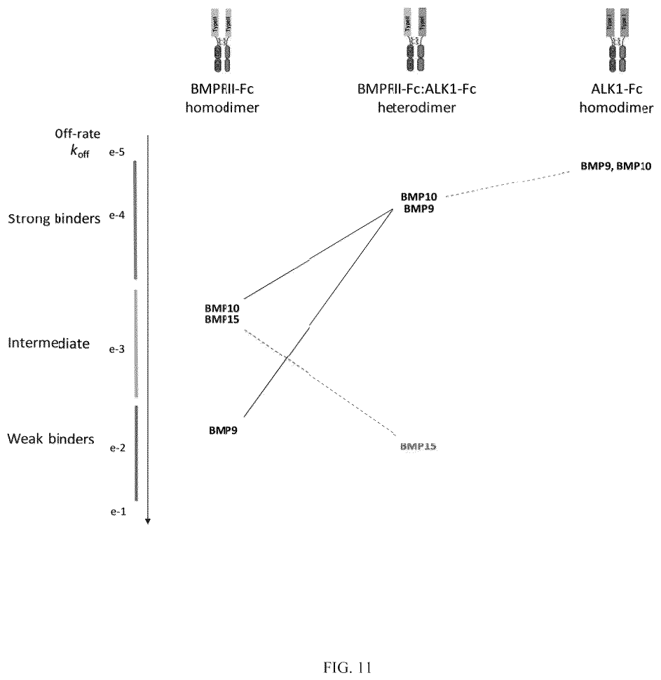

These comparative binding data demonstrate that the ActRIIA-Fc:ALK4-Fc heterodimer has an altered binding profile/selectivity relative to either the ActRIIA-Fc or ALK4-Fc homodimers. For example, the ActRIIA-Fc:ALK4-Fc heterodimer exhibits enhanced binding to activin A, and particularly enhanced binding to activin AC, compared to ActRIIA-Fc homodimer, while retaining strong binding to activin AB and GDF11. In addition, the ligand with highest affinity for ActRIIA-Fc homodimer, activin B, displays reduced affinity (albeit still within the high-affinity range) for the ActRIIA-Fc:ALK4-Fc heterodimer. The ActRIIA-Fc:ALK4-Fc heterodimer also exhibits markedly reduced binding to BMP10 compared to ActRIIA-Fc homodimer. See FIG. 10.

These results demonstrate that the ActRIIA-Fc:ALK4-Fc heterodimer is a more selective antagonist of activin A and activin AB over activin B than is ActRIIA-Fc homodimer. In addition, the ActRIIA-Fc:ALK4-Fc heterodimer has substantially increased affinity for activin AC and greatly reduced affinity for BMP10 compared to ActRIIA-Fc homodimer. Accordingly, an ActRIIA-Fc:ALK4-Fc heterodimer will be more useful than ActRIIA-Fc homodimer in certain applications where such selective antagonism is advantageous. Examples include therapeutic applications where it is desirable to antagonize activin A and/or activin AB preferentially over activin B, and to obtain strong inhibition of activin AC, while avoiding inhibition of BMP10.

### Example 16. Generation of a BMPRII-Fc:ALK1-Fc Heterodimer

Applicants constructed a soluble BMPRII-Fc:ALK1-Fc heteromeric complex comprising the extracellular domains of human BMPRII and human ALK1, which are each separately fused to an Fc domain with a linker positioned between the extracellular domain and the Fc domain. The individual constructs are referred to as BMPRII-Fc fusion polypeptide and ALK1-Fc fusion polypeptide, respectively, and the sequences for each are provided below.

Formation of heteromeric BMPRII-Fc:ALK1-Fc may be guided by approaches similar to those described in Example 1. In a first approach, one Fc domain is altered to introduce cationic amino acids at the interaction face, while the other Fc domain is altered to introduce anionic amino acids at the interaction face.

The BMPRII-Fc polypeptide sequence (SEQ ID NO: 121) is shown below:

| (SEQ ID NO: 121) | |
|---|---|
| 1 | MDAMKRGLCC VLLLCGAVFV SPGASQNQER LCAFKDPYQQ DLGIGESRIS |
| 51 | HENGTILCSK GSTCYGLWEK SKGDINLVKQ GCWSHIGDPQ ECHYEECVVT |
| 101 | TTPPSIQNGT YRFCCCSTDL CNVNFTENFP PPDTTPLSPP HSFNRDETGG |
| 151 | GTHTCPPCPA PELLGGPSVF LFPPKPKDTL MISRTPEVTC VVVDVSHEDP |
| 201 | EVKFNWYVDG VEVHNAKTKP REEQYNSTYR VVSVLTVLHQ DWLNGKEYKC |
| 251 | KVSNKALPAP IEKTISKAKG QPREPQVYTL PPSRKEMTKN QVSLTCLVKG |
| 301 | FYPSDIAVEW ESNGQPENNY KTTPPVLKSD GSFFLYSKLT VDKSRWQQGN |
| 351 | VFSCSVMHEA LHNHYTQKSL SLSPGK |

The leader sequence and linker sequence are underlined. To promote formation of the BMPRII-Fc:ALK1-Fc heterodimer rather than either of the possible homodimeric complexes, two amino acid substitutions (replacing acidic amino acids with lysine) can be introduced into the Fc domain of the BMPRII-Fc fusion protein as indicated by double underline above. The amino acid sequence of SEQ ID NO: 121 may optionally be provided with the lysine removed from the C-terminus.

This BMPRII-Fc fusion protein is encoded by the following nucleic acid sequence (SEQ ID NO: 122):

| (SEQ ID NO: 122) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCGCAGAA TCAAGAACGC CTATGTGCGT |
| 101 | TTAAAGATCC GTATCAGCAA GACCTTGGGA TAGGTGAGAG TAGAATCTCT |
| 151 | CATGAAAATG GGACAATATT ATGCTCGAAA GGTAGCACCT GCTATGGCCT |
| 201 | TTGGGAGAAA TCAAAAGGGG ACATAAATCT TGTAAAACAA GGATGTTGGT |
| 251 | CTCACATTGG AGATCCCCAA GAGTGTCACT ATGAAGAATG TGTAGTAACT |
| 301 | ACCACTCCTC CCTCAATTCA GAATGGAACA TACCGTTTCT GCTGTTGTAG |
| 351 | CACAGATTTA TGTAATGTCA ACTTTACTGA GAATTTTCCA CCTCCTGACA |
| 401 | CAACACCACT CAGTCCACCT CATTCATTTA ACCGAGATGA GACCGGTGGT |
| 451 | GGAACTCACA CATGCCCACC GTGCCCAGCA CCTGAACTCC TGGGGGGACC |
| 501 | GTCAGTCTTC CTCTTCCCCC CAAAACCCAA GGACACCCTC ATGATCTCCC |
| 551 | GGACCCCTGA GGTCACATGC GTGGTGGTGG ACGTGAGCCA CGAAGACCCT |
| 601 | GAGGTCAAGT TCAACTGGTA CGTGGACGGC GTGGAGGTGC ATAATGCCAA |
| 651 | GACAAAGCCG CGGGAGGAGC AGTACAACAG CACGTACCGT GTGGTCAGCG |
| 701 | TCCTCACCGT CCTGCACCAG GACTGGCTGA ATGGCAAGGA GTACAAGTGC |
| 751 | AAGGTCTCCA ACAAAGCCCT CCCAGCCCCC ATCGAGAAAA CCATCTCCAA |
| 801 | AGCCAAAGGG CAGCCCCGAG AACCACAGGT GTACACCCTG CCCCCATCCC |
| 851 | GGAAGGAGAT GACCAAGAAC CAGGTCAGCC TGACCTGCCT GGTCAAAGGC |
| 901 | TTCTATCCCA GCGACATCGC CGTGGAGTGG GAGAGCAATG GGCAGCCGGA |
| 951 | GAACAACTAC AAGACCACGC CTCCCGTGCT GAAGTCCGAC GGCTCCTTCT |
| 1001 | TCCTCTATAG CAAGCTCACC GTGGACAAGA GCAGGTGGCA GCAGGGGAAC |
| 1051 | GTCTTCTCAT GCTCCGTGAT GCATGAGGCT CTGCACAACC ACTACACGCA |
| 1101 | GAAGAGCCTC TCCCTGTCTC CGGGTAAA |

The mature BMPRII-Fc fusion polypeptide (SEQ ID NO: 123) is as follows and may optionally be provided with the lysine removed from the C-terminus.

| (SEQ ID NO: 123) | |
|---|---|
| 1 | SQNQERLCAF KDPYQQDLGI GESRISHENG TILCSKGSTC YGLWEKSKGD |
| 51 | INLVKQGCWS HIGDPQECHY EECVVTTTPP SIQNGTYRFC CCSTDLCNVN |
| 101 | FTENFPPPDT TPLSPPHSFN RDETGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVYTLPPSR KEMTKNQVSL TCLVKGFYPS DIAVEWESNG QPENNYKTTP |
| 301 | PVLKSDGSFF LYSKLTVDKS RWQQGNVFSC SVMHEALHNH YTQKSLSLSP |
| 351 | GK |

The complementary form of ALK1-Fc fusion polypeptide (SEQ ID NO: 124) is as follows:

| (SEQ ID NO: 124) | |
|---|---|
| 1 | MDAMKRGLCC VLLLCGAVFV SPGADPVKPS RGPLVTCTCE SPHCKGPTCR |
| 51 | GAWCTVVLVR EEGRHPQEHR GCGNLHRELC RGRPTEFVNH YCCDSHLCNH |
| 101 | NVSLVLEATQ PPSEQPGTDG QLATGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVYTLPPSR EEMTKNQVSL TCLVKGFYPS DIAVEWESNG QPENNYDTTP |
| 301 | PVLDSDGSFF LYSDLTVDKS RWQQGNVFSC SVMHEALHNH YTQKSLSLSP |
| 351 | G |

The leader sequence and linker sequence are underlined. To guide heterodimer formation with the BMPRII-Fc fusion polypeptide of SEQ ID NOs: 121 and 123 above, two amino acid substitutions (replacing lysines with aspartic acids) can be introduced into the Fc domain of the ALK1-Fc fusion polypeptide as indicated by double underline above. The amino acid sequence of SEQ ID NO: 124 may optionally be provided with a lysine added at the C-terminus.

This ALK1-Fc fusion protein is encoded by the following nucleic acid (SEQ ID NO: 125):

| (SEQ ID NO: 125) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGACCCTGT GAAGCCGTCT CGGGGCCCGC |
| 101 | TGGTGACCTG CACGTGTGAG AGCCCACATT GCAAGGGGCC TACCTGCCGG |
| 151 | GGGGCCTGGT GCACAGTAGT GCTGGTGCGG GAGGAGGGGA GGCACCCCCA |
| 201 | GGAACATCGG GGCTGCGGGA ACTTGCACAG GGAGCTCTGC AGGGGCCGCC |
| 251 | CCACCGAGTT CGTCAACCAC TACTGCTGCG ACAGCCACCT CTGCAACCAC |
| 301 | AACGTGTCCC TGGTGCTGGA GGCCACCCAA CCTCCTTCGG AGCAGCCGGG |
| 351 | AACAGATGGC CAGCTGGCCA CCGGTGGTGG AACTCACACA TGCCCACCGT |
| 401 | GCCCAGCACC TGAACTCCTG GGGGGACCGT CAGTCTTCCT CTTCCCCCCA |
| 451 | AAACCCAAGG ACACCCTCAT GATCTCCCGG ACCCCTGAGG TCACATGCGT |
| 501 | GGTGGTGGAC GTGAGCCACG AAGACCCTGA GGTCAAGTTC AACTGGTACG |
| 551 | TGGACGGCGT GGAGGTGCAT AATGCCAAGA CAAAGCCGCG GGAGGAGCAG |
| 601 | TACAACAGCA CGTACCGTGT GGTCAGCGTC CTCACCGTCC TGCACCAGGA |
| 651 | CTGGCTGAAT GGCAAGGAGT ACAAGTGCAA GGTCTCCAAC AAAGCCCTCC |
| 701 | CAGCCCCCAT CGAGAAAACC ATCTCCAAAG CCAAAGGGCA GCCCCGAGAA |
| 751 | CCACAGGTGT ACACCCTGCC CCCATCCCGG GAGGAGATGA CCAAGAACCA |
| 801 | GGTCAGCCTG ACCTGCCTGG TCAAAGGCTT CTATCCCAGC GACATCGCCG |
| 851 | TGGAGTGGGA GAGCAATGGG CAGCCGGAGA ACAACTACGA CACCACGCCT |
| 901 | CCCGTGCTGG ACTCCGACGG CTCCTTCTTC CTCTATAGCG ACCTCACCGT |
| 951 | GGACAAGAGC AGGTGGCAGC AGGGGAACGT CTTCTCATGC TCCGTGATGC |
| 1001 | ATGAGGCTCT GCACAACCAC TACACGCAGA AGAGCCTCTC CCTGTCTCCG |
| 1051 | GGT |

The mature ALK1-Fc fusion protein sequence (SEQ ID NO: 126) is as follows and may optionally be provided with a lysine added at the C-terminus.

| (SEQ ID NO: 126) | |
|---|---|
| 1 | DPVKPSRGPL VTCTCESPHC KGPTCRGAWC TVVLVREEGR HPQEHRGCGN |
| 51 | LHRELCRGRP TEFVNHYCCD SHLCNHNVSL VLEATQPPSE QPGTDGQLAT |
| 101 | GGGTHTCPPC PAPELLGGPS VFLFPPKPKD TLMISRTPEV TCVVVDVSHE |
| 151 | DPEVKFNWYV DGVEVHNAKT KPREEQYNST YRVVSVLTVL HQDWLNGKEY |
| 201 | KCKVSNKALP APIEKTISKA KGQPREPQVY TLPPSREEMT KNQVSLTCLV |
| 251 | KGFYPSDIAV EWESNGQPEN NYDTTPPVLD SDGSFFLYSD LTVDKSRWQQ |
| 301 | GNVFSCSVMH EALHNHYTQK SLSLSPG |

The BMPRII-Fc and ALK1-Fc proteins of SEQ ID NO: 123 and SEQ ID NO: 126, respectively, may be co-expressed and purified from a CHO cell line, to give rise to a heteromeric complex comprising BMPRII-Fc:ALK1-Fc.

In a second approach to promote the formation of heteromultimer complexes using asymmetric Fc fusion proteins, the Fc domains are altered to introduce complementary hydrophobic interactions and an additional intermolecular disulfide bond.

The BMPRII-Fc polypeptide sequence (SEQ ID NO: 411) is shown below:

| (SEQ ID NO: 411) | |
|---|---|
| 1 | MDAMKRGLCC VLLLCGAVFV SPGASQNQER LCAFKDPYQQ DLGIGESRIS |
| 51 | HENGTILCSK GSTCYGLWEK SKGDINLVKQ GCWSHIGDPQ ECHYEECVVT |
| 101 | TTPPSIQNGT YRFCCCSTDL CNVNFTENFP PPDTTPLSPP HSFNRDETGG |
| 151 | GTHTCPPCPA PELLGGPSVF LFPPKPKDTL MISRTPEVTC VVVDVSHEDP |
| 201 | EVKFNWYVDG VEVHNAKTKP REEQYNSTYR VVSVLTVLHQ DWLNGKEYKC |
| 251 | KVSNKALPAP IEKTISKAKG QPREPQVYTL PPCREEMTKN QVSLWCLVKG |
| 301 | FYPSDIAVEW ESNGQPENNY KTTPPVLDSD GSFFLYSKLT VDKSRWQQGN |
| 351 | VFSCSVMHEA LHNHYTQKSL SLSPGK |

The leader sequence and linker sequence are underlined. To promote formation of the BMPRII-Fc:ALK1-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 411 may optionally be provided with the lysine removed from the C-terminus.

The mature BMPRII-Fc fusion polypeptide (SEQ ID NO: 412) is as follows and may optionally be provided with the lysine (K) removed from the C-terminus.

| (SEQ ID NO: 412) | |
|---|---|
| 1 | SQNQERLCAF KDPYQQDLGI GESRISHENG TILCSKGSTC YGLWEKSKGD |
| 51 | INLVKQGCWS HIGDPQECHY EECVVTTTPP SIQNGTYRFC CCSTDLCNVN |
| 101 | FTENFPPPDT TPLSPPHSFN RDETGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVYTLPPCR EEMTKNQVSL WCLVKGFYPS DIAVEWESNG QPENNYKTTP |
| 301 | PVLDSDGSFF LYSKLTVDKS RWQQGNVFSC SVMHEALHNH YTQKSLSLSP |
| 351 | GK |

The complementary form of ALK1-Fc fusion polypeptide (SEQ ID NO: 413) is as follows:

| (SEQ ID NO: 413) | |
|---|---|
| 1 | MDAMKRGLCC VLLLCGAVFV SPGADPVKPS RGPLVTCTCE SPHCKGPTCR |
| 51 | GAWCTVVLVR EEGRHPQEHR GCGNLHRELC RGRPTEFVNH YCCDSHLCNH |
| 101 | NVSLVLEATQ PPSEQPGTDG QLATGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVCTLPPSR EEMTKNQVSL SCAVKGFYPS DIAVEWESNG QPENNYKTTP |
| 301 | PVLDSDGSFF LVSKLTVDKS RWQQGNVFSC SVMHEALHNH YTQKSLSLSP |
| 351 | GK |

The leader sequence and linker sequence are underlined. To guide heterodimer formation with the BMPRII-Fc fusion polypeptide of SEQ ID NOs: 411 and 412 above, four amino acid substitutions can be introduced into the Fc domain of the ALK1 fusion polypeptide as indicated by double underline above. The amino acid sequence of SEQ ID NO: 413 may optionally be provided with the lysine removed from the C-terminus.

The mature ALK1-Fc fusion protein sequence (SEQ ID NO: 414) is as follows and may optionally be provided with the lysine removed from the C-terminus.

| (SEQ ID NO: 414) | |
|---|---|
| 1 | DPVKPSRGPL VTCTCESPHC KGPTCRGAWC TVVLVREEGR HPQEHRGCGN |
| 51 | LHRELCRGRP TEFVNHYCCD SHLCNHNVSL VLEATQPPSE QPGTDGQLAT |
| 101 | GGGTHTCPPC PAPELLGGPS VFLFPPKPKD TLMISRTPEV TCVVVDVSHE |
| 151 | DPEVKFNWYV DGVEVHNAKT KPREEQYNST YRVVSVLTVL HQDWLNGKEY |
| 201 | KCKVSNKALP APIEKTISKA KGQPREPQVC TLPPSREEMT KNQVSLSCAV |
| 251 | KGFYPSDIAV EWESNGQPEN NYKTTPPVLD SDGSFFLVSK LTVDKSRWQQ |
| 301 | GNVFSCSVMH EALHNHYTQK SLSLSPGK |

The BMPRII-Fc and ALK1-Fc proteins of SEQ ID NO: 412 and SEQ ID NO: 414, respectively, may be co-expressed and purified from a CHO cell line, to give rise to a heteromeric complex comprising BMPRII-Fc:ALK1-Fc.

Purification of various BMPRII-Fc:ALK1-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 17. Ligand Binding Profile of BMPRII-Fc:ALK1-Fc Heterodimer Compared to BMPRII-Fc Homodimer and ALK1-Fc Homodimer

A Biacore™-based binding assay was used to compare ligand binding selectivity of the BMPRII-Fc:ALK1-Fc heterodimeric complex described above with that of BMPRII-Fc and ALK1-Fc homodimeric complexes. The BMPRII-Fc:ALK1-Fc heterodimer, BMPRII-Fc homodimer, and ALK1-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand binding profile of BMPRII-Fc:ALK1-Fc heterodimer
compared to BMPRII-Fc homodimer and ALK1-Fc homodimer

| | BMPRII-Fc | | | ALK1-Fc | | | BMPRII-Fc:ALK1-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |
| BMP9 | 1.2 × 10⁷ | 2.6 × 10⁻² | 2100 | 7.8 × 10⁶ | 1.3 × 10⁻⁴ | 16 | 1.2 × 10⁶ | 4.1 × 10⁻⁴ | 360 |
| BMP10 | 2.6 × 10⁷ | 2.5 × 10⁻³ | 100 | 4.1 × 10⁶ | 1.6 × 10⁻⁴ | 38 | 1.5 × 10⁷ | 3.5 × 10⁻⁴ | 23 |

| | | | | | | | |
|---|---|---|---|---|---|---|---|
| BMP15 | 9.9 × 10⁶ | 2.8 × 10⁻³ | 290 | No binding | 1.2 × 10⁷ | 4.2 × 10⁻² | 3500 |

These comparative binding data demonstrate that the BMPRII-Fc:ALK1-Fc heterodimer has a binding profile/selectivity which differs from that of BMPRII-Fc homodimer but is similar to that of ALK1-Fc homodimer. For example, the BMPRII-Fc:ALK1-Fc heterodimer largely retains the strong binding to BMP9 and BMP10 characteristic of ALK1-Fc homodimer; however, the heterodimer displays modest selectivity for BMP10 over BMP9 not present with the homodimer. Also unlike ALK1-Fc homodimer, the BMPRII-Fc:ALK1-Fc heterodimer binds to BMP15, albeit with an affinity approximately an order of magnitude weaker than that of BMPRII-Fc homodimer. See FIG. 11. Accordingly, a BMPRII-Fc:ALK1-Fc heterodimer will be unexpectedly useful in certain therapeutic applications where selective antagonism of BMP9 and particularly BMP10 is advantageous, e.g., for inhibition of angiogenesis, or in applications where antagonism of BMP15 is also advantageous.

### Example 18. Generation of a BMPRII-Fc:ALK2-Fc Heterodimer

Applicants constructed a soluble BMPRII-Fc:ALK2-Fc heteromeric complex comprising the extracellular domains of human BMPRII and human ALK2, which are each separately fused to an Fc domain with a linker positioned between the extracellular domain and the Fc domain. The individual constructs are referred to as BMPRII-Fc fusion polypeptide and ALK2-Fc fusion polypeptide, respectively, and the sequences for each are provided herein.

Formation of heteromeric BMPRII-Fc:ALK2-Fc may be guided by approaches similar to those described in Example 1. In a first approach, one Fc domain is altered to introduce cationic amino acids at the interaction face, while the other Fc domain is altered to introduce anionic amino acids at the interaction face. The polypeptide sequence of the BMPRII-Fc fusion polypeptide and a nucleic acid sequence encoding it are provided above in Example 16 as SEQ ID NOs: 121-123. To promote formation of the BMPRII-Fc:ALK2-Fc heterodimer rather than either of the possible homodimeric complexes, two amino acid substitutions (replacing acidic amino acids with lysine) can be introduced into the Fc domain of the BMPRII-Fc fusion protein as indicated in Example 16. The amino acid sequences of SEQ ID NOs: 121 and 123 may optionally be provided with the lysine removed from the C-terminus.

The polypeptide sequence of the complementary ALK2-Fc fusion polypeptide and a nucleic acid sequence encoding it are provided in Example 9 as SEQ ID NOs: 136-138. To guide heterodimer formation with the BMPRII-Fc fusion polypeptide of SEQ ID NOs: 121 and 123, two amino acid substitutions (replacing lysines with aspartic acids) can be introduced into the Fc domain of the ALK2-Fc fusion polypeptide as indicated in Example 9. The amino acid sequences of SEQ ID NOs: 136 and 138 may optionally be provided with a lysine added at the C-terminus.

The BMPRII-Fc and ALK2-Fc fusion polypeptides of SEQ ID NO: 123 and SEQ ID NO: 138, respectively, may be co-expressed and purified from a CHO cell line, to give rise to a heteromeric complex comprising BMPRII-Fc:ALK2-Fc.

In a second approach to promote the formation of heteromultimer complexes using asymmetric Fc fusion proteins, the Fc domains are altered to introduce complementary hydrophobic interactions and an additional intermolecular disulfide bond. BMPRII-Fc fusion polypeptide sequences (SEQ ID NOs: 411-412) are discussed in Example 16. To promote formation of the BMPRII-Fc:ALK2-Fc heterodimer rather than either of the possible homodimeric complexes, two amino acid substitutions (replacing a serine with a cysteine and a threonine with a trytophan) can be introduced into the Fc domain of the BMPRII-Fc polypeptide as indicated in Example 16. The amino acid sequences of SEQ ID NOs: 411 and 412 may optionally be provided with the lysine removed from the C-terminus.

Polypeptide sequences of the complementary ALK2-Fc fusion polypeptide (SEQ ID NOs: 421-422) are discussed in Example 9. To guide heterodimer formation with the BMPRII-Fc fusion polypeptide of SEQ ID NOs: 411-412, four amino acid substitutions can be introduced into the Fc domain of the ALK2 fusion polypeptide as indicated in Example 9. The amino acid sequences of SEQ ID NOs: 421-422 may optionally be provided with the lysine removed from the C-terminus.

The BMPRII-Fc and ALK2-Fc fusion polypeptides of SEQ ID NO: 412 and SEQ ID NO: 422, respectively, may be co-expressed and purified from a CHO cell line, to give rise to a heteromeric complex comprising BMPRII-Fc:ALK2-Fc.

Purification of various BMPRII-Fc:ALK2-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 19. Ligand Binding Profile of BMPRII-Fc:ALK2-Fc Heterodimer Compared to BMPRII-Fc Homodimer and ALK2-Fc Homodimer

A Biacore™-based binding assay was used to compare ligand binding selectivity of the BMPRII-Fc:ALK2-Fc heterodimeric complex described above with that of BMPRII-Fc and ALK2-Fc homodimeric complexes. The BMPRII-Fc:ALK2-Fc heterodimer, BMPRII-Fc homodimer, and ALK2-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted by gray shading.

Ligand binding profile of BMPRII-Fc:ALK2-Fc heterodimer
compared to BMPRII-Fc homodimer and ALK2-Fc homodimer

| | BMPRII-Fc | | | ALK2-Fc | | | BMPRII-Fc:ALK2-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |

| Activin B | 1.9 × 10⁶ | 4.9 × 10⁻³ | 2600 | No binding | 5.9 × 10⁵ | 3.1 × 10⁻³ | 5200 |
|---|---|---|---|---|---|---|---|
| BMP5 | 1.9 × 10⁶ | 1.9 × 10⁻² | 9900 | No binding | 1.8 × 10⁶ | 5.0 × 10⁻³ | 2800 |
| BMP7 | Transient* | | >93000 | No binding | 1.5 × 10⁷ | 1.2 × 10⁻² | 760 |
| BMP9 | 4.5 × 10⁷ | 7.3 × 10⁻² | 1600 | No binding | 1.0 × 10⁷ | 5.1 × 10⁻³ | 500 |
| BMP10 | 3.8 × 10⁷ | 5.0 × 10⁻³ | 130 | No binding | 1.1 × 10⁸ | 3.4 × 10⁻² | 300 |
| BMP15 | 5.8 × 10⁶ | 4.2 × 10⁻³ | 720 | No binding | 9.6 × 10⁶ | 1.1 × 10⁻² | 1100 |

*Indeterminate due to transient nature of interaction

### Example 20. Generation of a BMPRII-Fc:ALK3-Fc Heterodimer

Applicants constructed a soluble BMPRII-Fc:ALK3-Fc heteromeric complex comprising the extracellular domains of human BMPRII and human ALK3, which are each separately fused to an Fc domain with a linker positioned between the extracellular domain and the Fc domain. The individual constructs are referred to as BMPRII-Fc fusion polypeptide and ALK3-Fc fusion polypeptide, respectively, and the sequences for each are provided herein.

Formation of heteromeric BMPRII-Fc:ALK3-Fc may be guided by approaches similar to those described in Example 1. In a first approach, one Fc domain is altered to introduce cationic amino acids at the interaction face, while the other Fc domain is altered to introduce anionic amino acids at the interaction face. The polypeptide sequence of the BMPRII-Fc fusion polypeptide and a nucleic acid sequence encoding it are provided above in Example 16 as SEQ ID NOs: 121-123. To promote formation of the BMPRII-Fc:ALK3-Fc heterodimer rather than either of the possible homodimeric complexes, two amino acid substitutions (replacing acidic amino acids with lysine) can be introduced into the Fc domain of the BMPRII-Fc fusion protein as indicated in Example 16. The amino acid sequences of SEQ ID NOs: 121 and 123 may optionally be provided with the lysine removed from the C-terminus.

The polypeptide sequence of the complementary ALK3-Fc fusion polypeptide and a nucleic acid sequence encoding it are provided in Example 4 as SEQ ID NOs: 115-117. To guide heterodimer formation with the BMPRII-Fc fusion polypeptide of SEQ ID NOs: 121 and 123, two amino acid substitutions (replacing lysines with aspartic acids) can be introduced into the Fc domain of the ALK3-Fc fusion polypeptide as indicated in Example 4. The amino acid sequences of SEQ ID NOs: 115 and 117 may optionally be provided with a lysine added at the C-terminus.

The BMPRII-Fc and ALK3-Fc fusion polypeptides of SEQ ID NO: 123 and SEQ ID NO: 117, respectively, may be co-expressed and purified from a CHO cell line, to give rise to a heteromeric complex comprising BMPRII-Fc:ALK3-Fc.

In a second approach to promote the formation of heteromultimer complexes using asymmetric Fc fusion proteins, the Fc domains are altered to introduce complementary hydrophobic interactions and an additional intermolecular disulfide bond. BMPRII-Fc fusion polypeptide sequences (SEQ ID NOs: 411-412) are discussed in Example 16. To promote formation of the BMPRII-Fc:ALK3-Fc heterodimer rather than either of the possible homodimeric complexes, two amino acid substitutions (replacing a serine with a cysteine and a threonine with a trytophan) can be introduced into the Fc domain of the BMPRII-Fc polypeptide as indicated in Example 16. The amino acid sequences of SEQ ID NOs: 411 and 412 may optionally be provided with the lysine removed from the C-terminus.

Polypeptide sequences of the complementary ALK3-Fc fusion polypeptide (SEQ ID NOs: 407-408) are discussed in Example 4. To guide heterodimer formation with the BMPRII-Fc fusion polypeptide of SEQ ID NOs: 411-412, four amino acid substitutions can be introduced into the Fc domain of the ALK3 fusion polypeptide as indicated in Example 4. The amino acid sequences of SEQ ID NOs: 407 and 408 may optionally be provided with the lysine removed from the C-terminus.

The BMPRII-Fc and ALK3-Fc fusion polypeptides of SEQ ID NO: 412 and SEQ ID NO: 408, respectively, may be co-expressed and purified from a CHO cell line, to give rise to a heteromeric complex comprising BMPRII-Fc:ALK3-Fc.

Purification of various BMPRII-Fc:ALK3-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 21. Ligand Binding Profile of BMPRII-Fc:ALK3-Fc Heterodimer Compared to BMPRII-Fc Homodimer and ALK3-Fc Homodimer

A Biacore™-based binding assay was used to compare ligand binding selectivity of the BMPRII-Fc:ALK3-Fc heterodimeric complex described above with that of BMPRII-Fc and ALK3-Fc homodimeric complexes. The BMPRII-Fc:ALK3-Fc heterodimer, BMPRII-Fc homodimer, and ALK3-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand binding profile of BMPRII-Fc:ALK3-Fc heterodimer
compared to BMPRII-Fc homodimer and ALK3-Fc homodimer

| | BMPRII-Fc | | | ALK3-Fc | | | BMPRII-Fc:ALK3-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |

| | | | | | |
|---|---|---|---|---|---|
| Activin B | 2.0 × 10⁷ | 7.5 × 10⁻² | 3800 | No binding | Minimal binding |

| BMP2 | Transient* | >2 × 10⁶ | 6.2 × 10⁵ | 1.4 × 10⁻⁴ | 230 | 2.9 × 10⁶ | 1.5 × 10⁻⁴ | 51 |
|---|---|---|---|---|---|---|---|---|
| BMP4 | — | | 2.6 × 10⁵ | 5.5 × 10⁻⁵ | 210 | 9.1 × 10⁵ | 9.1 × 10⁻⁵ | 100 |
| BMP5 | — | | 2.9 × 10⁴ | 2.3 × 10⁻³ | 70000 | 4.3 × 10⁵ | 1.4 × 10⁻³ | 3200 |
| BMP6 | Transient* | >8900 | 1.4 × 10⁵ | 4.9 × 10⁻³ | 35000 | 3.6 × 10⁵ | 5.9 × 10⁻⁴ | 1600 |
| BMP7 | Transient* | >38000 | 1.2 × 10⁶ | 1.8 × 10⁻² | 15000 | 1.2 × 10⁷ | 1.2 × 10⁻² | 1000 |

| BMP9 | 1.2 × 10⁷ | 2.6 × 10⁻² | 2100 | No binding | No binding | | |
|---|---|---|---|---|---|---|---|
| BMP10 | 2.6 × 10⁷ | 2.5 × 10⁻³ | 100 | — | 6.8 × 10⁵ | 1.6 × 10⁻³ | 2400 |
| BMP15 | 9.9 × 10⁶ | 2.8 × 10⁻³ | 290 | — | 9.1 × 10⁵ | 5.5 × 10⁻³ | 6000 |

| GDF5 | No binding | | 4.3 × 10⁵ | 1.1 × 10⁻² | 22000 | Minimal binding | | |
|---|---|---|---|---|---|---|---|---|
| GDF6 | Transient* | >88000 | 3.4 × 10⁴ | 1.3 × 10⁻³ | 40000 | 1.4 × 10⁶ | 1.9 × 10⁻³ | 1400 |

*Indeterminate due to transient nature of interaction
— Not tested

These comparative binding data demonstrate that the BMPRII-Fc:ALK3-Fc heterodimer has ligand binding selectivity which is clearly unlike that of BMPRII-Fc homodimer but also differs from that of ALK3-Fc homodimer. BMPRII-Fc:ALK3-Fc heterodimer binds much more strongly to BMP6 than does ALK3-Fc homodimer, reflecting an off-rate nearly ten-fold slower. With its largely unchanged binding to BMP2 and BMP4, the BMPRII-Fc:ALK3 heterodimer can therefore be considered a joint inhibitor of BMP2, BMP4, and BMP6. This binding profile contrasts with that of ALK3-Fc homodimer, whose exceptionally strongly binding to BMP4 and BMP2 identifies it as highly selective for this ligand pair compared to four ligands with intermediate-level binding, including BMP6. See FIG. 12. Accordingly, a BMPRII-Fc:ALK3-Fc heterodimer will be unexpectedly useful in certain therapeutic applications where joint antagonism of BMP2, BMP4, and BMP6 is advantageous.

### Example 22. Generation of a BMPRII-Fc:ALK4-Fc Heterodimer

Applicants constructed a soluble BMPRII-Fc:ALK4-Fc heteromeric complex comprising the extracellular domains of human BMPRII and human ALK4, which are each separately fused to an Fc domain with a linker positioned between the extracellular domain and the Fc domain. The individual constructs are referred to as BMPRII-Fc fusion polypeptide and ALK4-Fc fusion polypeptide, respectively, and the sequences for each are provided herein.

Formation of heteromeric BMPRII-Fc:ALK4-Fc may be guided by approaches similar to those described in Example 1. In a first approach, one Fc domain is altered to introduce cationic amino acids at the interaction face, while the other Fc domain is altered to introduce anionic amino acids at the interaction face. The polypeptide sequence of the BMPRII-Fc fusion polypeptide and a nucleic acid sequence encoding it are provided above in Example 16 as SEQ ID NOs: 121-123. To promote formation of the BMPRII-Fc:ALK4-Fc heterodimer rather than either of the possible homodimeric complexes, two amino acid substitutions (replacing acidic amino acids with lysine) can be introduced into the Fc domain of the BMPRII-Fc fusion protein as indicated in Example 16. The amino acid sequences of SEQ ID NOs: 121 and 123 may optionally be provided with the lysine removed from the C-terminus.

The polypeptide sequence of the complementary ALK4-Fc fusion polypeptide and a nucleic acid sequence encoding it are provided in Example 1 as SEQ ID NOs: 104-106. To guide heterodimer formation with the BMPRII-Fc fusion polypeptide of SEQ ID NOs: 121 and 123, two amino acid substitutions (replacing lysines with aspartic acids) can be introduced into the Fc domain of the ALK4-Fc fusion polypeptide as indicated in Example 1. The amino acid sequences of SEQ ID NOs: 104 and 106 may optionally be provided with a lysine added at the C-terminus.

The BMPRII-Fc and ALK4-Fc fusion polypeptides of SEQ ID NO: 123 and SEQ ID NO: 106, respectively, may be co-expressed and purified from a CHO cell line, to give rise to a heteromeric complex comprising BMPRII-Fc:ALK4-Fc.

In a second approach to promote the formation of heteromultimer complexes using asymmetric Fc fusion proteins, the Fc domains are altered to introduce complementary hydrophobic interactions and an additional intermolecular disulfide bond. BMPRII-Fc fusion polypeptide sequences (SEQ ID NOs: 411 and 412) are discussed in Example 16. To promote formation of the BMPRII-Fc:ALK4-Fc heterodimer rather than either of the possible homodimeric complexes, two amino acid substitutions (replacing a serine with a cysteine and a threonine with a trytophan) can be introduced into the Fc domain of the BMPRII-Fc polypeptide as indicated in Example 16. The amino acid sequences of SEQ ID NOs: 411 and 412 may optionally be provided with the lysine removed from the C-terminus.

Polypeptide sequences of the complementary ALK4-Fc fusion polypeptide (SEQ ID NOs: 403 and 404) are discussed in Example 1. To guide heterodimer formation with the BMPRII-Fc fusion polypeptide of SEQ ID NOs: 411 and 412, four amino acid substitutions can be introduced into the Fc domain of the ALK4 fusion polypeptide as indicated in Example 1. The amino acid sequences of SEQ ID NOs: 403 and 404 may optionally be provided with the lysine removed from the C-terminus.

The BMPRII-Fc and ALK4-Fc fusion polypeptides of SEQ ID NO: 412 and SEQ ID NO: 404, respectively, may be co-expressed and purified from a CHO cell line, to give rise to a heteromeric complex comprising BMPRII-Fc:ALK4-Fc.

Purification of various BMPRII-Fc:ALK4-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 23. Ligand Binding Profile of BMPRII-Fc:ALK4-Fc Heterodimer Compared to BMPRII-Fc Homodimer and ALK4-Fc Homodimer

A Biacore™-based binding assay was used to compare ligand binding selectivity of the BMPRII-Fc:ALK4-Fc heterodimeric complex described above with that of BMPRII-Fc and ALK4-Fc homodimeric complexes. The BMPRII-Fc:ALK4-Fc heterodimer, BMPRII-Fc homodimer, and ALK4-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand binding profile of BMPRII-Fc:ALK4-Fc heterodimer
compared to BMPRII-Fc homodimer and ALK4-Fc homodimer

| | BMPRII-Fc | | | ALK4-Fc | | | BMPRII-Fc:ALK4-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |
| Activin A | Transient* | | >43000 | 5.8 × 10⁵ | 1.2 × 10⁻² | 20000 | 2.0 × 10⁶ | 2.2 × 10⁻³ | 1100 |
| Activin B | 2.0 × 10⁷ | 7.5 × 10⁻² | 3800 | No binding | | | 1.6 × 10⁶ | 2.6 × 10⁻³ | 1700 |
| Activin AB | — | — | — | 4.4 × 10⁶ | 6.4 × 10⁻³ | 1500 | 3.6 × 10⁶ | 5.0 × 10⁻⁴ | 140 |

| BMP9 | 1.2 × 10⁷ | 2.6 × 10⁻² | 2100 | No binding | Transient* | | >140000 |
|---|---|---|---|---|---|---|---|
| BMP10 | 2.6 × 10⁷ | 2.5 × 10⁻³ | 100 | No binding | 8.0 × 10⁵ | 1.8 × 10⁻³ | 2200 |
| BMP15 | 9.9 × 10⁶ | 2.8 × 10⁻³ | 290 | No binding | 2.8 × 10⁷ | 4.8 × 10⁻² | 1700 |

*Indeterminate due to transient nature of interaction
— Not tested

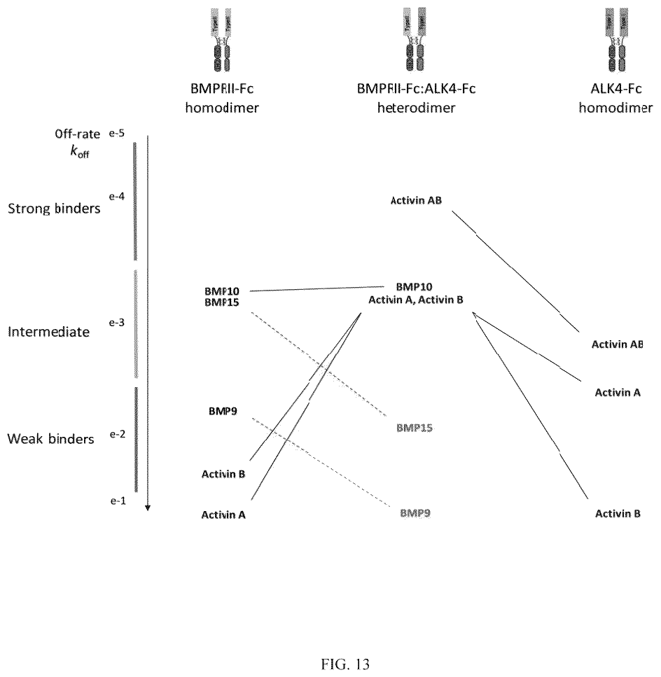

These comparative binding data demonstrate that the BMPRII-Fc:ALK4-Fc heterodimer has ligand binding selectivity which is unlike that of either BMPRII-Fc homodimer or ALK4-Fc homodimer. BMPRII-Fc:ALK4-Fc heterodimer differs from both homodimers by binding several activin ligands with high or intermediate strength and differs from BMPRII-Fc homodimer by binding BMP15 only weakly. Most notably, BMPRII-Fc:ALK4-Fc heterodimer binds strongly and preferentially to the heterodimeric ligand activin AB. See FIG. 13. Accordingly, a BMPRII-Fc:ALK4-Fc heterodimer will be unexpectedly useful in certain therapeutic applications where antagonism of activin A, activin B, and particularly activin AB are advantageous and where antagonism of BMP15 (which is heavily implicated in ovulation) is to be avoided.

### Example 24. Generation of a TGFβRII-Fc:ALK1-Fc Heterodimer

Applicants constructed a soluble TGFβRII-Fc:ALK1-Fc heteromeric complex comprising the extracellular domains of the short (canonical) isoform of human TGFβRII and human ALK1, which are each separately fused to an Fc domain with a linker positioned between the extracellular domain and the Fc domain. The individual constructs are referred to as TGFβRII_{SHORT}-Fc fusion polypeptide and ALK1-Fc fusion polypeptide, respectively, and the sequences for each are provided herein.

Formation of heteromeric TGFβRII_{SHORT}-Fc:ALK1-Fc may be guided by approaches similar to those described in Example 1. In a first approach, one Fc domain is altered to introduce cationic amino acids at the interaction face, while the other Fc domain is altered to introduce anionic amino acids at the interaction face.

The TGFβRII_{SHORT}-Fc polypeptide sequence (SEQ ID NO: 127) is shown below:

| (SEQ ID NO: 127) | |
|---|---|
| 1 | MDAMKRGLCC VLLLCGAVFV SPGATIPPHV QKSVNNDMIV TDNNGAVKFP |
| 51 | QLCKFCDVRF STCDNQKSCM SNCSITSICE KPQEVCVAVW RKNDENITLE |
| 101 | TVCHDPKLPY HDFILEDAAS PKCIMKEKKK PGETFFMCSC SSDECNDNII |
| 151 | FSEEYNTSNP DTGGGTHTCP PCPAPELLGG PSVFLFPPKP KDTLMISRTP |
| 201 | EVTCVVVDVS HEDPEVKFNW YVDGVEVHNA KTKPREEQYN STYRVVSVLT |
| 251 | VLHQDWLNGK EYKCKVSNKA LPAPIEKTIS KAKGQPREPQ VYTLPPSRKE |
| 301 | MTKNQVSLTC LVKGFYPSDI AVEWESNGQP ENNYKTTPPV LKSDGSFFLY |
| 351 | SKLTVDKSRW QQGNVFSCSV MHEALHNHYT QKSLSLSPGK |

The leader sequence and linker sequence are underlined. To promote formation of the TGFβRII_{SHORT}-Fc:ALK1-Fc heterodimer rather than either of the possible homodimeric complexes, two amino acid substitutions (replacing acidic amino acids with lysine) can be introduced into the Fc domain of the TGFβRII_{SHORT}-Fc fusion protein as indicated by double underline above. The amino acid sequence of SEQ ID NO: 127 may optionally be provided with the lysine removed from the C-terminus.

This TGFβRII_{SHORT}-Fc fusion protein is encoded by the following nucleic acid sequence (SEQ ID NO: 128):

| (SEQ ID NO: 128) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | TTAATAACGA CATGATAGTC ACTGACAACA ACGGTGCAGT CAAGTTTCCA |
| 151 | CAACTGTGTA AATTTTGTGA TGTGAGATTT TCCACCTGTG ACAACCAGAA |
| 201 | ATCCTGCATG AGCAACTGCA GCATCACCTC CATCTGTGAG AAGCCACAGG |
| 251 | AAGTCTGTGT GGCTGTATGG AGAAAGAATG ACGAGAACAT AACACTAGAG |
| 301 | ACAGTTTGCC ATGACCCCAA GCTCCCCTAC CATGACTTTA TTCTGGAAGA |
| 351 | TGCTGCTTCT CCAAAGTGCA TTATGAAGGA AAAAAAAAAG CCTGGTGAGA |
| 401 | CTTTCTTCAT GTGTTCCTGT AGCTCTGATG AGTGCAATGA CAACATCATC |
| 451 | TTCTCAGAAG AATATAACAC CAGCAATCCT GACACCGGTG GTGGAACTCA |
| 501 | CACATGCCCA CCGTGCCCAG CACCTGAACT CCTGGGGGGA CCGTCAGTCT |
| 551 | TCCTCTTCCC CCCAAAACCC AAGGACACCC TCATGATCTC CCGGACCCCT |
| 601 | GAGGTCACAT GCGTGGTGGT GGACGTGAGC CACGAAGACC CTGAGGTCAA |
| 651 | GTTCAACTGG TACGTGGACG GCGTGGAGGT GCATAATGCC AAGACAAAGC |
| 701 | CGCGGGAGGA GCAGTACAAC AGCACGTACC GTGTGGTCAG CGTCCTCACC |
| 751 | GTCCTGCACC AGGACTGGCT GAATGGCAAG GAGTACAAGT GCAAGGTCTC |
| 801 | CAACAAAGCC CTCCCAGCCC CCATCGAGAA AACCATCTCC AAAGCCAAAG |
| 851 | GGCAGCCCCG AGAACCACAG GTGTACACCC TGCCCCCATC CCGGAAGGAG |
| 901 | ATGACCAAGA ACCAGGTCAG CCTGACCTGC CTGGTCAAAG GCTTCTATCC |
| 951 | CAGCGACATC GCCGTGGAGT GGGAGAGCAA TGGGCAGCCG GAGAACAACT |
| 1001 | ACAAGACCAC GCCTCCCGTG CTGAAGTCCG ACGGCTCCTT CTTCCTCTAT |
| 1051 | AGCAAGCTCA CCGTGGACAA GAGCAGGTGG CAGCAGGGGA ACGTCTTCTC |
| 1101 | ATGCTCCGTG ATGCATGAGG CTCTGCACAA CCACTACACG CAGAAGAGCC |
| 1151 | TCTCCCTGTC TCCGGGTAAA |

The mature TGFβRII_{SHORT}-Fc fusion polypeptide (SEQ ID NO: 129) is as follows and may optionally be provided with the lysine removed from the C-terminus.

| (SEQ ID NO: 129) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE YNTSNPDTGG GTHTCPPCPA |
| 151 | PELLGGPSVF LFPPKPKDTL MISRTPEVTC VVVDVSHEDP EVKFNWYVDG |
| 201 | VEVHNAKTKP REEQYNSTYR VVSVLTVLHQ DWLNGKEYKC KVSNKALPAP |
| 251 | IEKTISKAKG QPREPQVYTL PPSRKEMTKN QVSLTCLVKG FYPSDIAVEW |
| 301 | ESNGQPENNY KTTPPVLKSD GSFFLYSKLT VDKSRWQQGN VFSCSVMHEA |
| 351 | LHNHYTQKSL SLSPGK |

The polypeptide sequence of the complementary ALK1-Fc fusion polypeptide and a nucleic acid sequence encoding it are provided in Example 16 as SEQ ID NOs: 124-126. To guide heterodimer formation with the TGFβRII_{SHORT}-Fc fusion polypeptide of SEQ ID NOs: 127 and 129, two amino acid substitutions (replacing lysines with aspartic acids) can be introduced into the Fc domain of the ALK1-Fc fusion polypeptide as indicated in Example 16. The amino acid sequences of SEQ ID NOs: 124 and 126 may optionally be provided with a lysine added at the C-terminus.

The TGFβRII_{SHORT}-Fc and ALK1-Fc proteins of SEQ ID NO: 129 and SEQ ID NO: 126, respectively, may be co-expressed and purified from a CHO cell line, to give rise to a heteromeric complex comprising TGFβRII_{SHORT}-Fc:ALK1-Fc.

A variant TGFβII-Fc:ALK1-Fc heteromeric complex may be generated in which the ALK1-Fc polypeptide described above (SEQ ID NO: 126) is paired with an Fc fusion protein comprising the extracellular domain of the long (A) isoform of TGFβRII (TGFβRII_{LONG}) in place of the extracellular domain of the short isoform.

The TGFβRII_{LONG}-Fc polypeptide sequence (SEQ ID NO: 130) is shown below:

| (SEQ ID NO: 130) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG THTCPPCPAP |
| 201 | ELLGGPSVFL FPPKPKDTLM ISRTPEVTCV VVDVSHEDPE VKFNWYVDGV |
| 251 | EVHNAKTKPR EEQYNSTYRV VSVLTVLHQD WLNGKEYKCK VSNKALPAPI |
| 301 | EKTISKAKGQ PREPQVYTLP PSRKEMTKNQ VSLTCLVKGF YPSDIAVEWE |
| 351 | SNGQPENNYK TTPPVLKSDG SFFLYSKLTV DKSRWQQGNV FSCSVMHEAL |
| 401 | HNHYTQKSLS LSPGK |

The leader sequence and linker sequence are underlined. To promote formation of the TGFβRII_{LONG}-Fc:ALK1-Fc heterodimer rather than either of the possible homodimeric complexes, two amino acid substitutions (replacing acidic amino acids with lysine) can be introduced into the Fc domain of the TGFβRII_{LONG}-Fc fusion protein as indicated by double underline above. The amino acid sequence of SEQ ID NO: 130 may optionally be provided with the lysine removed from the C-terminus.

This TGFβRII_{LONG}-Fc fusion protein is encoded by the following nucleic acid sequence (SEQ ID NO: 131):

| (SEQ ID NO: 131) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA ACTCACACAT GCCCACCGTG CCCAGCACCT |
| 601 | GAACTCCTGG GGGGACCGTC AGTCTTCCTC TTCCCCCCAA AACCCAAGGA |
| 651 | CACCCTCATG ATCTCCCGGA CCCCTGAGGT CACATGCGTG GTGGTGGACG |
| 701 | TGAGCCACGA AGACCCTGAG GTCAAGTTCA ACTGGTACGT GGACGGCGTG |
| 751 | GAGGTGCATA ATGCCAAGAC AAAGCCGCGG GAGGAGCAGT ACAACAGCAC |
| 801 | GTACCGTGTG GTCAGCGTCC TCACCGTCCT GCACCAGGAC TGGCTGAATG |
| 851 | GCAAGGAGTA CAAGTGCAAG GTCTCCAACA AAGCCCTCCC AGCCCCCATC |
| 901 | GAGAAAACCA TCTCCAAAGC CAAAGGGCAG CCCCGAGAAC CACAGGTGTA |
| 951 | CACCCTGCCC CCATCCCGGA AGGAGATGAC CAAGAACCAG GTCAGCCTGA |
| 1001 | CCTGCCTGGT CAAAGGCTTC TATCCCAGCG ACATCGCCGT GGAGTGGGAG |
| 1051 | AGCAATGGGC AGCCGGAGAA CAACTACAAG ACCACGCCTC CCGTGCTGAA |
| 1101 | GTCCGACGGC TCCTTCTTCC TCTATAGCAA GCTCACCGTG GACAAGAGCA |
| 1151 | GGTGGCAGCA GGGGAACGTC TTCTCATGCT CCGTGATGCA TGAGGCTCTG |
| 1201 | CACAACCACT ACACGCAGAA GAGCCTCTCC CTGTCTCCGG GTAAA |

The mature TGFβRII_{LONG}-Fc fusion polypeptide (SEQ ID NO: 132) is as follows and may optionally be provided with the lysine removed from the C-terminus.

| (SEQ ID NO: 132) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PDTGGGTHTC PPCPAPELLG GPSVFLFPPK PKDTLMISRT |
| 201 | PEVTCVVVDV SHEDPEVKFN WYVDGVEVHN AKTKPREEQY NSTYRVVSVL |
| 251 | TVLHQDWLNG KEYKCKVSNK ALPAPIEKTI SKAKGQPREP QVYTLPPSRK |
| 301 | EMTKNQVSLT CLVKGFYPSD IAVEWESNGQ PENNYKTTPP VLKSDGSFFL |
| 351 | YSKLTVDKSR WQQGNVFSCS VMHEALHNHY TQKSLSLSPG K |

In a second approach to promote the formation of heteromultimer complexes using asymmetric Fc fusion proteins, the Fe domains are altered to introduce complementary hydrophobic interactions and an additional intermolecular disulfide bond.

The TGFβRII_{SHORT}-Fc polypeptide sequence (SEQ ID NO: 415) is shown below:

| (SEQ ID NO: 415) | |
|---|---|
| 1 | MDAMKRGLCC VLLLCGAVFV SPGATIPPHV QKSVNNDMIV TDNNGAVKFP |
| 51 | QLCKFCDVRF STCDNQKSCM SNCSITSICE KPQEVCVAVW RKNDENITLE |
| 101 | TVCHDPKLPY HDFILEDAAS PKCIMKEKKK PGETFFMCSC SSDECNDNII |
| 151 | FSEEYNTSNP DTGGGTHTCP PCPAPELLGG PSVFLFPPKP KDTLMISRTP |
| 201 | EVTCVVVDVS HEDPEVKFNW YVDGVEVHNA KTKPREEQYN STYRVVSVLT |
| 251 | VLHQDWLNGK EYKCKVSNKA LPAPIEKTIS KAKGQPREPQ VYTLPPCREE |
| 301 | MTKNQVSLWC LVKGFYPSDI AVEWESNGQP ENNYKTTPPV LDSDGSFFLY |
| 351 | SKLTVDKSRW QQGNVFSCSV MHEALHNHYT QKSLSLSPGK |

The leader sequence and linker sequence are underlined. To promote formation of the TGFβRII_{SHORT}-Fc:ALK1-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 415 may optionally be provided with the lysine removed from the C-terminus.

The mature TGFβRII_{SHORT}-Fc fusion polypeptide (SEQ ID NO: 416) is as follows and may optionally be provided with the lysine removed from the C-terminus.

| (SEQ ID NO: 416) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE YNTSNPDTGG GTHTCPPCPA |
| 151 | PELLGGPSVF LFPPKPKDTL MISRTPEVTC VVVDVSHEDP EVKFNWYVDG |
| 201 | VEVHNAKTKP REEQYNSTYR VVSVLTVLHQ DWLNGKEYKC KVSNKALPAP |
| 251 | IEKTISKAKG QPREPQVYTL PPCREEMTKN QVSLWCLVKG FYPSDIAVEW |
| 301 | ESNGQPENNY KTTPPVLDSD GSFFLYSKLT VDKSRWQQGN VFSCSVMHEA |
| 351 | LHNHYTQKSL SLSPGK |

Polypeptide sequences of the complementary ALK1-Fc fusion polypeptide (SEQ ID NOs: 413 and 414) are discussed in Example 16. To guide heterodimer formation with the TGFβRII_{SHORT}-Fc fusion polypeptide of SEQ ID NOs: 415 and 416, four amino acid substitutions can be introduced into the Fc domain of the ALK1 fusion polypeptide as indicated in Example 16. The amino acid sequences of SEQ ID NOs: 413 and 414 may optionally be provided with the lysine removed from the C-terminus.

The TGFβRII_{SHORT}-Fc and ALK1-Fc proteins of SEQ ID NO: 416 and SEQ ID NO: 414, respectively, may be co-expressed and purified from a CHO cell line, to give rise to a heteromeric complex comprising TGFβRII-Fc:ALK1-Fc.

A variant TGFβRII-Fc:ALK1-Fc heteromeric complex may be generated in which the ALK1-Fc polypeptide described above (SEQ ID NO: 414) is paired with an Fc fusion protein comprising the extracellular domain of the long (A) isoform of TGFβRII (TGFβRII_{LONG}) in place of the extracellular domain of the short isoform.

The TGFβRII_{LONG}-Fc polypeptide sequence (SEQ ID NO: 417) is shown below:

| (SEQ ID NO: 417) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG THTCPPCPAP |
| 201 | ELLGGPSVFL FPPKPKDTLM ISRTPEVTCV VVDVSHEDPE VKFNWYVDGV |
| 251 | EVHNAKTKPR EEQYNSTYRV VSVLTVLHQD WLNGKEYKCK VSNKALPAPI |
| 301 | EKTISKAKGQ PREPQVYTLP PCREEMTKNQ VSLWCLVKGF YPSDIAVEWE |
| 351 | SNGQPENNYK TTPPVLDSDG SFFLYSKLTV DKSRWQQGNV FSCSVMHEAL |
| 401 | HNHYTQKSLS LSPGK |

The leader sequence and linker sequence are underlined. To promote formation of the TGFβRII_{LONG}-Fc:ALK1-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 417 may optionally be provided with the lysine removed from the C-terminus.

The mature TGFβRII_{LONG}-Fc fusion polypeptide (SEQ ID NO: 418) is as follows and may optionally be provided with the lysine removed from the C-terminus.

| (SEQ ID NO: 418) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PDTGGGTHTC PPCPAPELLG GPSVFLFPPK PKDTLMISRT |
| 201 | PEVTCVVVDV SHEDPEVKFN WYVDGVEVHN AKTKPREEQY NSTYRVVSVL |
| 251 | TVLHQDWLNG KEYKCKVSNK ALPAPIEKTI SKAKGQPREP QVYTLPPCRE |
| 301 | EMTKNQVSLW CLVKGFYPSD IAVEWESNGQ PENNYKTTPP VLDSDGSFFL |
| 351 | YSKLTVDKSR WQQGNVFSCS VMHEALHNHY TQKSLSLSPG K |

The TGFβRII_{LONG}-Fc and ALK1-Fc proteins of SEQ ID NO: 418 and SEQ ID NO: 414, respectively, may be co-expressed and purified from a CHO cell line, to give rise to a heteromeric complex comprising TGFβRII_{LONG}-Fc:ALK1-Fc.

Purification of various TGFβRII-Fc:ALK1-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 25. Ligand Binding Profile of TGFβRII-Fc:ALK1-Fc Heterodimer Compared to TGFβRII-Fc Homodimer and ALK1-Fc Homodimer

A Biacore™-based binding assay was used to compare ligand binding selectivity of the TGFβRII_{SHORT}-Fc:ALK1-Fc heterodimeric complex described above with that of TGFβRII_{SHORT}-Fc and ALK1-Fc homodimeric complexes. The TGFβRII_{SHORT}-Fc:ALK1-Fc heterodimer, TGFβRII_{SHORT}-Fc homodimer, and ALK1-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand binding profile of TGFBRII_{SHORT}-Fc:ALK1-Fc heterodimer
compared to TGFBRII_{SHORT}-Fc homodimer and ALK1-Fc homodimer

| | TGFBRII_{SHORT}-Fc | | | ALK1-Fc | | | TGFBRII_{SHORT}-Fc:ALK1-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |

| | | | | | | | |
|---|---|---|---|---|---|---|---|
| BMP9 | No binding | 7.9 × 10⁶ | 1.3 × 10⁻⁴ | 16 | 2.1 × 10⁷ | 2.2 × 10⁻³ | 110 |
| BMP10 | No binding | 1.7 × 10⁷ | 1.1 × 10⁻⁴ | 6 | 1.2 × 10⁷ | 9.6 × 10⁻⁴ | 78 |

| TGFβ1 | 4.2 × 10⁷ | 1.1 × 10⁻³ | 25 | No binding | Transient* | >5300 |
|---|---|---|---|---|---|---|
| TGFβ2 | Transient* | | >44000 | No binding | No binding | |
| TGFβ3 | 5.9 × 10⁷ | 5.9 × 10⁻³ | 99 | No binding | Transient* | >4700 |

*Indeterminate due to transient nature of interaction

### Example 26. Generation of a TGFβRII_{SHORT}-Fc:ALK5-Fc Heterodimer

Applicants constructed a soluble TGFβRII_{SHORT}-Fc:ALK5-Fc heteromeric complex comprising the extracellular domains of the human TGFβRII short (canonical) isoform and human ALK5, which are each separately fused to an Fc domain with a linker positioned between the extracellular domain and the Fc domain. The individual constructs are referred to as TGFβRII_{SHORT}-Fc fusion polypeptide and ALK5-Fc fusion polypeptide, respectively, and the sequences for each are provided herein.

Formation of heteromeric TGFβRII_{SHORT}-Fc:ALK5-Fc may be guided by approaches similar to those described in Example 1. In a first approach, one Fc domain is altered to introduce cationic amino acids at the interaction face, while the other Fc domain is altered to introduce anionic amino acids at the interaction face. The polypeptide sequence of the TGFβRII_{SHORT}-Fc fusion polypeptide and a nucleic acid sequence encoding it are provided above in Example 24 as SEQ ID NOs: 127-129. To promote formation of the TGFβRII_{SHORT}-Fc:ALK5-Fc heterodimer rather than either of the possible homodimeric complexes, two amino acid substitutions (replacing acidic amino acids with lysine) can be introduced into the Fc domain of the TGFβRII_{SHORT}-Fc fusion protein as indicated in Example 24. The amino acid sequences of SEQ ID NOs: 127 and 129 may optionally be provided with the lysine removed from the C-terminus.

The polypeptide sequence of the complementary ALK5-Fc fusion polypeptide and a nucleic acid sequence encoding it are provided in Example 11 as SEQ ID NOs: 139-141. To guide heterodimer formation with the TGFβRII_{SHORT}-Fc fusion polypeptide of SEQ ID NOs: 127 and 129, two amino acid substitutions (replacing lysines with aspartic acids) can be introduced into the Fc domain of the ALK5-Fc fusion polypeptide as indicated in Example 11. The amino acid sequences of SEQ ID NOs: 139 and 141 may optionally be provided with a lysine added at the C-terminus.

The TGFβRII_{SHORT}-Fc and ALK5-Fc fusion polypeptides of SEQ ID NO: 129 and SEQ ID NO: 141, respectively, may be co-expressed and purified from a CHO cell line, to give rise to a heteromeric complex comprising TGFβRII_{SHORT}-Fc:ALK5-Fc.

In a second approach to promote the formation of heteromultimer complexes using asymmetric Fc fusion proteins, the Fc domains are altered to introduce complementary hydrophobic interactions and an additional intermolecular disulfide bond. TGFβRII_{SHORT}-Fc fusion polypeptide sequences (SEQ ID NOs: 415-416) are discussed in Example 24. To promote formation of the TGFβRII_{SHORT}-Fc:ALK5-Fc heterodimer rather than either of the possible homodimeric complexes, two amino acid substitutions (replacing a serine with a cysteine and a threonine with a trytophan) can be introduced into the Fc domain of the TGFβRII_{SHORT}-Fc polypeptide as indicated in Example 24. The amino acid sequences of SEQ ID NOs: 415-416 may optionally be provided with the lysine removed from the C-terminus.

Polypeptide sequences of the complementary ALK5-Fc fusion polypeptide (SEQ ID NOs: 423-424) are discussed in Example 11. To guide heterodimer formation with the TGFβRII_{SHORT}-Fc fusion polypeptide of SEQ ID NOs: 415-416, four amino acid substitutions can be introduced into the Fc domain of the ALK5 fusion polypeptide as indicated in Example 11. The amino acid sequences of SEQ ID NOs: 423-424 may optionally be provided with the lysine removed from the C-terminus.

The TGFβRII_{SHORT}-Fc and ALK5-Fc fusion polypeptides of SEQ ID NO: 416 and SEQ ID NO: 424, respectively, may be co-expressed and purified from a CHO cell line, to give rise to a heteromeric complex comprising TGFβRII_{SHORT}-Fc:ALK5-Fc.

Purification of various TGFβRII_{SHORT}-Fc:ALK5-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 27. Generation of a TGFβRII_{LONG}-Fc:ALK5-Fc heterodimer

Applicants constructed a soluble TGFβRII_{LONG}-Fc:ALK5-Fc heteromeric complex comprising the extracellular domain of the long (A) isoform of human TGFβRII and the extracellular domain of human ALK5, which are each separately fused to an Fc domain with a linker positioned between the extracellular domain and the Fc domain. The individual constructs are referred to as TGFβRII_{LONG}-Fc fusion polypeptide and ALK5-Fc fusion polypeptide, respectively, and the sequences for each are provided herein.

Formation of heteromeric TGFβRII_{LONG}-Fc:ALK5-Fc may be guided by approaches similar to those described in Example 1. In a first approach, one Fc domain is altered to introduce cationic amino acids at the interaction face, while the other Fc domain is altered to introduce anionic amino acids at the interaction face. The polypeptide sequence of the TGFβRII_{LONG}-Fc fusion polypeptide and a nucleic acid sequence encoding it are provided above in Example 24 as SEQ ID NOs: 130-132. To promote formation of the TGFβRII_{LONG}-Fc:ALK5-Fc heterodimer rather than either of the possible homodimeric complexes, two amino acid substitutions (replacing acidic amino acids with lysine) can be introduced into the Fc domain of the TGFβRII_{LONG}-Fc fusion protein as indicated in Example 24. The amino acid sequences of SEQ ID NOs: 130 and 132 may optionally be provided with the lysine removed from the C-terminus.

The polypeptide sequence of the complementary ALK5-Fc fusion polypeptide and a nucleic acid sequence encoding it are provided in Example 11 as SEQ ID NOs: 139-141. To guide heterodimer formation with the TGFβRII_{LONG}-Fc fusion polypeptide of SEQ ID NOs: 130 and 132, two amino acid substitutions (replacing lysines with aspartic acids) can be introduced into the Fc domain of the ALK5-Fc fusion polypeptide as indicated in Example 11. The amino acid sequences of SEQ ID NOs: 139 and 142 may optionally be provided with a lysine added at the C-terminus.

The TGFβRII_{LONG}-Fc and ALK5-Fc fusion polypeptides of SEQ ID NO: 132 and SEQ ID NO: 141, respectively, may be co-expressed and purified from a CHO cell line, to give rise to a heteromeric complex comprising TGFβRII_{LONG}-Fc:ALK5-Fc.

In a second approach to promote the formation of heteromultimer complexes using asymmetric Fc fusion proteins, the Fc domains are altered to introduce complementary hydrophobic interactions and an additional intermolecular disulfide bond. TGFβRII_{LONG}-Fc fusion polypeptide sequences (SEQ ID NOs: 417-418) are discussed in Example 24. To promote formation of the TGFβRII_{LONG}-Fc:ALK5-Fc heterodimer rather than either of the possible homodimeric complexes, two amino acid substitutions (replacing a serine with a cysteine and a threonine with a trytophan) can be introduced into the Fc domain of the TGFβRII_{LONG}-Fc polypeptide as indicated in Example 24. The amino acid sequences of SEQ ID NOs: 417-418 may optionally be provided with the lysine removed from the C-terminus.

Polypeptide sequences of the complementary ALK5-Fc fusion polypeptide (SEQ ID NOs: 423-424) are discussed in Example 11. To guide heterodimer formation with the TGFβRII_{LONG}-Fc fusion polypeptide of SEQ ID NOs: 417-418, four amino acid substitutions can be introduced into the Fc domain of the ALK5 fusion polypeptide as indicated in Example 11. The amino acid sequences of SEQ ID NOs: 423-424 may optionally be provided with the lysine removed from the C-terminus.

The TGFβRII_{LONG}-Fc and ALK5-Fc fusion polypeptides of SEQ ID NO: 418 and SEQ ID NO: 424, respectively, may be co-expressed and purified from a CHO cell line, to give rise to a heteromeric complex comprising TGFβRII_{LONG}-Fc:ALK5-Fc.

Purification of various TGFβRII_{LONG}-Fc:ALK5-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 28. Activity Profiles of TGFβRII-Fc:ALK5-Fc Heterodimers Compared to TGFβRII-Fc Homodimer and ALK5-Fc Homodimer

A Biacore™-based binding assay was used to compare ligand binding selectivity of the TGFβRII_{SHORT}-Fc:ALK5-Fc and TGFβRII_{LONG}-Fc:ALK5-Fc heterodimeric complexes described in Examples 26-27 with that of TGFβRII_{SHORT}-Fc and ALK5-Fc homodimeric complexes. The heteromeric or homomeric protein complexes were independently captured onto the system using an anti-Fc antibody. Ligands were injected and allowed to flow over the captured receptor protein. Results are summarized in the table below, in which ligand off-rates (k_{d}) most indicative of effective ligand traps are denoted in bold.

Ligand binding profiles of TGFβRII-Fc:ALK5-Fc heterodimers
compared to TGFβRII-Fc homodimer and ALK5-Fc homodimer

| | | | | TGFβRII_{SHORT}-Fc | | | TGFβRII_{SHORT}:ALK5-Fc | | | TGFβRII_{LONG}-Fc:ALK5-Fc | | |
|---|---|---|---|---|---|---|---|---|---|---|---|---|
| | ALK5-Fc | | | Homodimer | | | Heterodimer* | | | Heterodimer* | | |
| | Homodimer | | | kₐ | k_{d} | K_{D} | kₐ | k_{d} | K_{D} | kₐ | k_{d} | K_{D} |
| Ligand | kₐ | k_{d} | K_{D} | (1/Ms) | (1/s) | (pM) | (1/Ms) | (1/s) | (pM) | (1/Ms) | (1/s) | (pM) |

| | | | | | | | | | | |
|---|---|---|---|---|---|---|---|---|---|---|
| TGFβ1 | No binding | 5.6 × 10⁷ | 1.1 × 10⁻³ | 20 | 1.4 × 10⁸ | 1.7 × 10⁻³ | 12 | 6.6 × 10⁷ | 9.2 × 10⁻⁴ | 14 |
| TGFβ2 | No binding | 2.1 × 10⁵ | 2.2 × 10⁻³ | 11000 | 6.6 × 10⁶ | 2.9 × 10⁻⁶ | 0.4 | 4.2 × 10⁶ | 2.8 × 10⁻⁷ | 0.07 |
| TGFβ3 | No binding | 1.9 × 10⁷ | 1.4 × 10⁻³ | 71 | 2.7 × 10⁷ | 1.0 × 10⁻³ | 38 | 2.7 × 10⁷ | 1.0 × 10⁻³ | 38 |

*Low signal which suggests that a substantial fraction of the protein is inactive

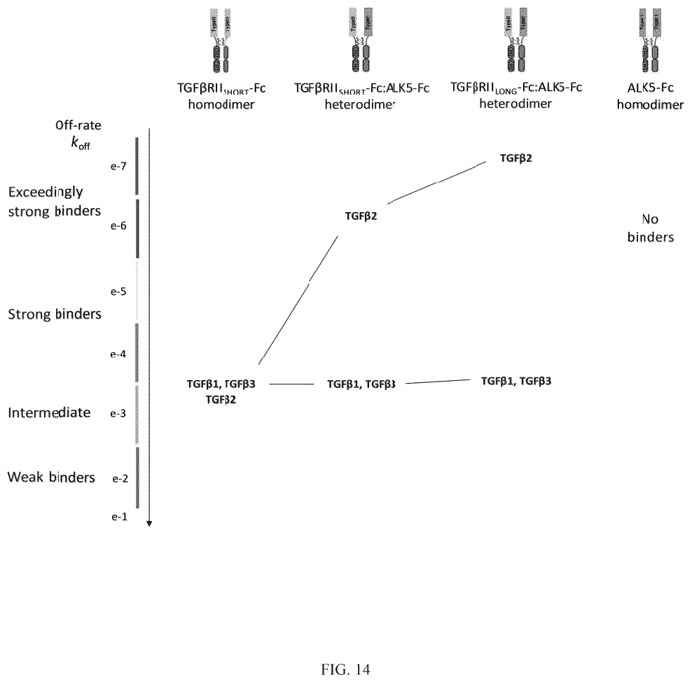

These comparative binding data indicate that the ligand binding profiles of TGFβRII-Fc:ALK5-Fc heterodimers are markedly different from that of TGFβRII-Fc homodimer and from ALK5-Fc homodimer, which did not bind any ligands. Based on the equilibrium dissociation constant (K_{D}), TGFβRII-Fc homodimer bound TGFβ1 and TGFβ3 with much higher affinity than TGFβ 1, even though off-rates for the three TGFβ ligands were similar. In contrast, TGFβRII-Fc:ALK5-Fc heterodimers displayed high selectivity for TGFβ2 over TGFβ1/TGFβ3. In particular, TGFβRII_{LONG}-Fc:ALK5-Fc heterodimer bound TGFβ2 with an affinity approximately five orders of magnitude higher and an off-rate approximately four orders of magnitude slower than did TGFβRII-Fc homodimer. TGFβRII_{LONG}-Fc:ALK5-Fc heterodimer also bound TGFβ2 more strongly than did heterodimer containing the short isoform. See FIG. 14. Neither of the TGFβRII-Fc:ALK5-Fc heterodimers was able to bind BMP9 or BMP10 (data not shown), which distinguishes these TGFβRII-Fc:ALK5-Fc heterodimers from TGFβRII-Fc:ALK1-Fc heterodimer (see Example 25). Sensograms for the two TGFβRII-Fc:ALK5-Fc heterodimers exhibited low signal amplitude which suggests that a substantial fraction of each protein was inactive.

To better interpret these data obtained by surface plasmon resonance, a reporter gene assay in A549 cells was used to determine the ability of TGFβRII fusion proteins to inhibit activity of TGF(31, TGFβ2, and TGFβ3. This assay is based on a human lung carcinoma cell line transfected with reporter plasmids pGL3(CAGA)12-firefly luciferase (Dennler et al, 1998, EMBO 17: 3091-3100) and pRLCMV-renilla luciferase, the latter to control for transfection efficiency. The CAGA motif is present in the promoters of TGFβ-responsive genes (for example, PAI-1), so this vector is of general use for factors signaling through SMAD2 and SMAD3.

On the first day of the assay, A549 cells (ATCC®: CCL-185™) were distributed in 48-well plates at 6.5×10⁴ cells per well and incubated overnight. All incubations were at 37° C. and 5% CO₂ in a tissue culture incubator unless otherwise indicated. On the second day, a solution containing 10 μg pGL3(CAGA)12-firefly luciferase, 100 ng pRLCMV-renilla luciferase, 30 μL X-tremeGENE 9 (Roche Applied Science), and 970 μL OptiMEM (Invitrogen) was preincubated for 30 min at room temperature, then added to 24 mL Eagle's minimum essential medium (EMEM, ATCC®) supplemented with 0.1% BSA. Medium was removed from the plated cells and this transfection mixture was applied to the cells (500 μl/well) for an overnight incubation. On the third day, medium was removed, and cells were incubated overnight with a mixture of ligands and inhibitors prepared as described below.

Serial dilutions of test articles were made in a 48-well plate in a 200 μL volume of assay buffer (EMEM+0.1% BSA). An equal volume of assay buffer containing the test ligand was added to obtain a final ligand concentration equal to the EC₅₀ determined previously. Human TGFβ1, TGFβ2, and TGFβ3 were obtained from PeproTech. Test solutions were incubated for 30 minutes, then 250 μL of the mixture was added to the transfected cells. Each concentration of test article was determined in duplicate. After incubation with test solutions overnight, cells were rinsed with phosphate-buffered saline, then lysed with passive lysis buffer (Promega E1941) and stored overnight at −70° C. On the fourth and final day, plates were warmed to room temperature with gentle shaking. Cell lysates were transferred to a chemiluminescence plate (96-well) and analyzed in a luminometer with reagents from a Dual-Luciferase Reporter Assay system (Promega E1980) to determine normalized luciferase activity.

This assay was used to compare the ability of TGFβRII fusion protein variants to inhibit cell signaling by TGFβRII ligands. Results are shown in the table below.

Inhibitory Activity of TGFβRII Fusion Proteins in A549 Cells

| | IC₅₀ (pM) | | |
|---|---|---|---|
| | TGFβ1 | TGFβ2 | TGFβ3 |
| Construct | (640 pg/mL) | (480 pg/mL) | (270 pg/mL) |
| TGFβRII_{SHORT}-Fc | 90 | — | 9 |
| homodimer | | | |
| TGFβRII_{SHORT}-Fc:ALK5-Fc | <350* | ~200* | <90* |
| heterodimer* | | | |
| TGFβRII_{LONG}-Fc:ALK5-Fc | 204 | 154 | 35 |
| heterodimer | | | |

— No inhibition (tested at concentrations up to 10 nM)
*Value imprecise due to range of concentrations tested

Results with TGFβRII-Fc homodimer were consistent with previous reports concerning wild-type TGFβRII_{SHORT}-Fc and TGFβRII_{LONG}-Fc homodimers (del Re et al., J Biol Chem 279:22765, 2004). In this experiment, TGFβRII_{SHORT}-Fc homodimer potently inhibited TGFβ 1 and TGFβ3 but was unable to inhibit TGFβ2 at homodimer concentrations up to 10 nM. This finding is consistent with the low affinity of TGFβ2 binding to TGFβRII-Fc homodimer but oddly inconsistent with its slow off-rate (see binding results above). In contrast, TGFβRII-Fc:ALK5-Fc heterodimers potently inhibited all three TGFβ ligands in a cellular environment. Accordingly, a TGFβRII-Fc:ALK5-Fc heterodimer will be unexpectedly useful in certain therapeutic applications where preferential antagonism of TGFβ2—or combined antagonism of TGFβ1, TGFβ2, and TGFβ3—are advantageous.

### Example 29. Generation of MISRII-Fc:ALK3-Fc Heterodimers

A soluble MISRII-Fc:ALK3-Fc heteromeric complex can be generated comprising the extracellular domains of human MISRII and human ALK3, which can each be separately fused to an Fc domain with a linker positioned between the extracellular domain and the Fc domain. The individual constructs are referred to as MISRII-Fc fusion polypeptide and ALK3-Fc fusion polypeptide, respectively.

Formation of heteromeric MISRII-Fc:ALK3-Fc may be guided by approaches similar to those described in Example 1. In a first approach, one Fc domain is altered to introduce cationic amino acids at the interaction face, while the other Fc domain is altered to introduce anionic amino acids at the interaction face.

The MISRII-Fc polypeptide sequence (SEQ ID NO: 133) is shown below:

| (SEQ ID NO: 133) | |
|---|---|
| 1 | MDAMKRGLCC VLLLCGAVFV SPGAPPNRRT CVFFEAPGVR GSTKTLGELL |
| 51 | DTGTELPRAI RCLYSRCCFG IWNLTQDRAQ VEMQGCRDSD EPGCESLHCD |
| 101 | PSPRAHPSPG STLFTCSCGT DFCNANYSHL PPPGSPGTPG SQGPQAAPGE |
| 151 | SIWMALTGGG THTCPPCPAP ELLGGPSVFL FPPKPKDTLM ISRTPEVTCV |
| 201 | VVDVSHEDPE VKFNWYVDGV EVHNAKTKPR EEQYNSTYRV VSVLTVLHQD |
| 251 | WLNGKEYKCK VSNKALPAPI EKTISKAKGQ PREPQVYTLP PSRKEMTKNQ |
| 301 | VSLTCLVKGF YPSDIAVEWE SNGQPENNYK TTPPVLKSDG SFFLYSKLTV |
| 351 | DKSRWQQGNV FSCSVMHEAL HNHYTQKSLS LSPGK |

The leader sequence and linker sequence are underlined. To promote formation of the MISRII-Fc:ALK3-Fc heterodimer rather than either of the possible homodimeric complexes, two amino acid substitutions (replacing acidic amino acids with lysine) can be introduced into the Fc domain of the MISRII fusion protein as indicated by double underline above. The amino acid sequence of SEQ ID NO: 133 may optionally be provided with the lysine removed from the C-terminus.

The mature MISRII-Fc fusion polypeptide (SEQ ID NO: 135) is as follows and may optionally be provided with the lysine removed from the C-terminus.

| (SEQ ID NO: 135) | |
|---|---|
| 1 | PPNRRTCVFF EAPGVRGSTK TLGELLDTGT ELPRAIRCLY SRCCFGIWNL |
| 51 | TQDRAQVEMQ GCRDSDEPGC ESLHCDPSPR AHPSPGSTLF TCSCGTDFCN |
| 101 | ANYSHLPPPG SPGTPGSQGP QAAPGESIWM ALTGGGTHTC PPCPAPELLG |
| 151 | GPSVFLFPPK PKDTLMISRT PEVTCVVVDV SHEDPEVKFN WYVDGVEVHN |
| 201 | AKTKPREEQY NSTYRVVSVL TVLHQDWLNG KEYKCKVSNK ALPAPIEKTI |
| 251 | SKAKGQPREP QVYTLPPSRK EMTKNQVSLT CLVKGFYPSD IAVEWESNGQ |
| 301 | PENNYKTTPP VLKSDGSFFL YSKLTVDKSR WQQGNVFSCS VMHEALHNHY |
| 351 | TQKSLSLSPG K |

In this first approach, the polypeptide sequence of the complementary ALK3-Fc fusion protein and a nucleic acid sequence encoding it are provided above in Example 4 as SEQ ID NOs: 115-117.

The MISRII-Fc and ALK3-Fc proteins of SEQ ID NO: 135 and SEQ ID NO: 117, respectively, may be co-expressed and purified from a CHO cell line, to give rise to a heteromeric complex comprising MISRII-Fc:ALK3-Fc. Similar heteromeric complexes may generated by pairing the ALK3-Fc protein described above (SEQ ID NO: 117) with an Fc fusion protein comprising the extracellular domain of MISRII isoform 2 or or the extracellular domain of MISRII isoform 3 in place of the extracellular domain of MISRII isoform 1.

In a second approach to promote the formation of heteromultimer complexes using asymmetric Fc fusion proteins, the Fc domains can be altered to introduce complementary hydrophobic interactions and an additional intermolecular disulfide bond.

The MISRII-Fc polypeptide sequence (SEQ ID NO: 419) is shown below:

| (SEQ ID NO: 419) | |
|---|---|
| 1 | MDAMKRGLCC VLLLCGAVFV SPGAPPNRRT CVFFEAPGVR GSTKTLGELL |
| 51 | DTGTELPRAI RCLYSRCCFG IWNLTQDRAQ VEMQGCRDSD EPGCESLHCD |
| 101 | PSPRAHPSPG STLFTCSCGT DFCNANYSHL PPPGSPGTPG SQGPQAAPGE |
| 151 | SIWMALTGGG THTCPPCPAP ELLGGPSVFL FPPKPKDTLM ISRTPEVTCV |
| 201 | VVDVSHEDPE VKFNWYVDGV EVHNAKTKPR EEQYNSTYRV VSVLTVLHQD |
| 251 | WLNGKEYKCK VSNKALPAPI EKTISKAKGQ PREPQVYTLP PCREEMTKNQ |
| 301 | VSLWCLVKGF YPSDIAVEWE SNGQPENNYK TTPPVLDSDG SFFLYSKLTV |
| 351 | DKSRWQQGNV FSCSVMHEAL HNHYTQKSLS LSPGK |

The leader sequence and linker sequence are underlined. To promote formation of the MISRII-Fc:ALK3-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 419 may optionally be provided with the lysine removed from the C-terminus.

The mature MISRII-Fc fusion polypeptide (SEQ ID NO: 420) is as follows and may optionally be provided with the lysine removed from the C-terminus.

| (SEQ ID NO: 420) | |
|---|---|
| 1 | PPNRRTCVFF EAPGVRGSTK TLGELLDTGT ELPRAIRCLY SRCCFGIWNL |
| 51 | TQDRAQVEMQ GCRDSDEPGC ESLHCDPSPR AHPSPGSTLF TCSCGTDFCN |
| 101 | ANYSHLPPPG SPGTPGSQGP QAAPGESIWM ALTGGGTHTC PPCPAPELLG |
| 151 | GPSVFLFPPK PKDTLMISRT PEVTCVVVDV SHEDPEVKFN WYVDGVEVHN |
| 201 | AKTKPREEQY NSTYRVVSVL TVLHQDWLNG KEYKCKVSNK ALPAPIEKTI |
| 251 | SKAKGQPREP QVYTLPPCRE EMTKNQVSLW CLVKGFYPSD IAVEWESNGQ |
| 301 | PENNYKTTPP VLDSDGSFFL YSKLTVDKSR WQQGNVFSCS VMHEALHNHY |
| 351 | TQKSLSLSPG K |

In this second approach, the polypeptide sequence of the complementary ALK3-Fc fusion protein and a nucleic acid sequence encoding it are provided above in Example 4 as SEQ ID NOs: 407-408.

The MISRII-Fc and ALK3-Fc proteins of SEQ ID NO: 420 and SEQ ID NO: 408, respectively, may be co-expressed and purified from a CHO cell line, to give rise to a heteromeric complex comprising MISRII-Fc:ALK3-Fc. Similar heteromeric complexes may be generated by pairing the ALK3-Fc protein described above (SEQ ID NO: 408) with an Fc fusion protein comprising the extracellular domain of MISRII isoform 2 or the extracellular domain of MISRII isoform 3 in place of the extracellular domain of MISRII isoform 1 described above.

Purification of various MISRII-Fc:ALK3-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 30. Generation of an ActRIIA-Fc:ActRIIB-Fc Heterodimer

A soluble ActRIIA-Fc:ActRIIB-Fc heteromeric complex can be generated comprising the extracellular domains of human ActRIIA and human ActRIIB, which are each fused to an Fc domain with a linker positioned between the extracellular domain and the Fc domain. The individual constructs are referred to as ActRIIA-Fc and ActRIIB-Fc fusion proteins, respectively.

Formation of heteromeric ActRIIA-Fc:ActRIIB-Fc may be guided by approaches similar to those described in Example 1.

### Electrostatic Approach

In a first approach, the polypeptide sequence of the ActRIIA-Fc fusion protein and a nucleic acid sequence encoding it are provided above in Example 14 as SEQ ID NOs: 118-120.

The polypeptide sequence of the complementary ActRIIB-Fc fusion protein (SEQ ID NO: 153) employs the TPA leader and is as follows:

| (SEQ ID NO: 153) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYDTTPPVLD SDGSFFLYSD LTVDKSRWQQ GNVFSCSVMH |
| 351 | EALHNHYTQK SLSLSPGK |

The leader and linker sequences are underlined. To promote formation of the ActRIIA-Fc:ActRIIB-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 153 may optionally be provided with the lysine removed from the C-terminus.

The mature ActRIIB-Fc fusion protein sequence is as follows (SEQ ID NO: 154) and may optionally be provided with the lysine removed from the C-terminus.

| (SEQ ID NO: 154) | |
|---|---|
| 1 | GRGEAETREC IYYNANWELE RTNQSGLERC EGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYDTT PPVLDSDGSF |
| 301 | FLYSDLTVDK SRWQQGNVFS CSVMHEALHN HYTQKSLSLS PGK |

The ActRIIA-Fc and ActRIIB-Fc fusion proteins of SEQ ID NO: 120 and SEQ ID NO: 154, respectively, may be co-expressed and purified from a CHO cell line to give rise to a heteromeric complex comprising ActRIIA-Fc:ActRIIB-Fc.

### Reverse Electrostatic Approach

In a reverse approach, receptor extracellular domains are both linked to the opposite member of the Fc interaction pair compared to the first approach described above. In this example, an alternative polypeptide sequence of the ActRIIA-Fc fusion protein (SEQ ID NO: 151) employs the TPA leader and is as follows.

| (SEQ ID NO: 151) | |
|---|---|
| 1 | MDAMKRGLCC VLLLCGAVFV SPGAAILGRS ETQECLFFNA NWEKDRTNQT |
| 51 | GVEPCYGDKD KRRHCFATWK NISGSIEIVK QGCWLDDINC YDRTDCVEKK |
| 101 | DSPEVYFCCC EGNMCNEKFS YFPEMEVTQP TSNPVTPKPP TGGGTHTCPP |
| 151 | CPAPELLGGP SVFLFPPKPK DTLMISRTPE VTCVVVDVSH EDPEVKFNWY |
| 201 | VDGVEVHNAK TKPREEQYNS TYRVVSVLTV LHQDWLNGKE YKCKVSNKAL |
| 251 | PAPIEKTISK AKGQPREPQV YTLPPSREEM TKNQVSLTCL VKGFYPSDIA |
| 301 | VEWESNGQPE NNYDTTPPVL DSDGSFFLYS DLTVDKSRWQ QGNVFSCSVM |
| 351 | HEALHNHYTQ KSLSLSPGK |

The leader and linker sequences are underlined. To promote formation of the ActRIIA-Fc:ActRIIB-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 151 may optionally be provided with the lysine removed from the C-terminus.

The mature ActRIIA-Fc fusion protein sequence is as follows (SEQ ID NO: 152) and may optionally be provided with the lysine removed from the C-terminus.

| (SEQ ID NO: 152) | |
|---|---|
| 1 | ILGRSETQEC LFFNANWEKD RTNQTGVEPC YGDKDKRRHC FATWKNISGS |
| 51 | IEIVKQGCWL DDINCYDRTD CVEKKDSPEV YFCCCEGNMC NEKFSYFPEM |
| 101 | EVTQPTSNPV TPKPPTGGGT HTCPPCPAPE LLGGPSVFLF PPKPKDTLMI |
| 151 | SRTPEVTCVV VDVSHEDPEV KFNWYVDGVE VHNAKTKPRE EQYNSTYRVV |
| 201 | SVLTVLHQDW LNGKEYKCKV SNKALPAPIE KTISKAKGQP REPQVYTLPP |
| 251 | SREEMTKNQV SLTCLVKGFY PSDIAVEWES NGQPENNYDT TPPVLDSDGS |
| 301 | FFLYSDLTVD KSRWQQGNVF SCSVMHEALH NHYTQKSLSL SPGK |

The polypeptide sequence of the ActRIIB-Fc fusion protein and a nucleic acid sequence encoding it are provided in Example 1 as SEQ ID NOs: 100-102.

The ActRIIA-Fc and ActRIIB-Fc fusion proteins of SEQ ID NO: 152 and SEQ ID NO: 102, respectively, may be co-expressed and purified from a CHO cell line to give rise to a variant heteromeric complex comprising ActRIIA-Fc:ActRIIB-Fc.

### Hydrophobic Approach

In another approach to promoting the formation of heteromultimer complexes using asymmetric Fc fusion proteins, the Fe domains are altered to introduce complementary hydrophobic interactions and an additional intermolecular disulfide bond. The polypeptide sequence of the ActRIIA-Fc fusion protein is provided in Example 14 as SEQ ID NOs: 409-410.

The complementary form of ActRIIB-Fc fusion polypeptide (SEQ ID NO: 453) is as follows:

| (SEQ ID NO: 453) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVC TLPPSREEMT KNQVSLSCAV KGFYPSDIAV |
| 301 | EWESNGQPEN NYKTTPPVLD SDGSFFLVSK LTVDKSRWQQ GNVFSCSVMH |
| 351 | EALHNHYTQK SLSLSPGK |

The leader and linker sequences are underlined. To guide heterodimer formation with the ActRIIA-Fc fusion polypeptide of SEQ ID NOs: 409-410, four amino acid substitutions can be introduced into the Fc domain of the ActRIIB-Fc fusion polypeptide as indicated by double underline above. The amino acid sequence of SEQ ID NO: 453 may optionally be provided with a lysine removed from the C-terminus.

The mature ActRIIB-Fc fusion protein sequence (SEQ ID NO: 454) is as follows and may optionally be provided with a lysine removed from the C-terminus.

| (SEQ ID NO: 454) | |
|---|---|
| 1 | GRGEAETREC IYYNANWELE RTNQSGLERC EGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVCTLPPS |
| 251 | REEMTKNQVS LSCAVKGFYP SDIAVEWESN GQPENNYKTT PPVLDSDGSF |
| 301 | FLVSKLTVDK SRWQQGNVFS CSVMHEALHN HYTQKSLSLS PGK |

The ActRIIA-Fc and ActRIIB-Fc proteins of SEQ ID NO: 410 and SEQ ID NO: 454, respectively, may be co-expressed and purified from a CHO cell line, to give rise to a variant heteromeric complex comprising ActRIIA-Fc:ActRIIB-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, an alternative polypeptide sequence of the ActRIIA-Fc fusion protein (SEQ ID NO: 451) employs the TPA leader and is as follows.

| (SEQ ID NO: 451) | |
|---|---|
| 1 | MDAMKRGLCC VLLLCGAVFV SPGAAILGRS ETQECLFFNA NWEKDRTNQT |
| 51 | GVEPCYGDKD KRRHCFATWK NISGSIEIVK QGCWLDDINC YDRTDCVEKK |
| 101 | DSPEVYFCCC EGNMCNEKFS YFPEMEVTQP TSNPVTPKPP TGGGTHTCPP |
| 151 | CPAPELLGGP SVFLFPPKPK DTLMISRTPE VTCVVVDVSH EDPEVKFNWY |
| 201 | VDGVEVHNAK TKPREEQYNS TYRVVSVLTV LHQDWLNGKE YKCKVSNKAL |
| 251 | PAPIEKTISK AKGQPREPQV CTLPPSREEM TKNQVSLSCA VKGFYPSDIA |
| 301 | VEWESNGQPE NNYKTTPPVL DSDGSFFLVS KLTVDKSRWQ QGNVFSCSVM |
| 351 | HEALHNHYTQ KSLSLSPGK |

The leader and linker sequences are underlined. To guide heterodimer formation with the ActRIIB-Fc fusion polypeptide of SEQ ID NOs 401-402, four amino acid substitutions can be introduced into the Fc domain of the ActRIIA-Fc fusion polypeptide as indicated by double underline above. The amino acid sequence of SEQ ID NO: 451 may optionally be provided with the lysine removed from the C-terminus.

The mature ActRIIA-Fc fusion protein sequence is as follows (SEQ ID NO: 452) and may optionally be provided with the lysine removed from the C-terminus.

| (SEQ ID NO: 452) | |
|---|---|
| 1 | ILGRSETQEC LFFNANWEKD RTNQTGVEPC YGDKDKRRHC FATWKNISGS |
| 51 | IEIVKQGCWL DDINCYDRTD CVEKKDSPEV YFCCCEGNMC NEKFSYFPEM |
| 101 | EVTQPTSNPV TPKPPTGGGT HTCPPCPAPE LLGGPSVFLF PPKPKDTLMI |
| 151 | SRTPEVTCVV VDVSHEDPEV KFNWYVDGVE VHNAKTKPRE EQYNSTYRVV |
| 201 | SVLTVLHQDW LNGKEYKCKV SNKALPAPIE KTISKAKGQP REPQVCTLPP |
| 251 | SREEMTKNQV SLSCAVKGFY PSDIAVEWES NGQPENNYKT TPPVLDSDGS |
| 301 | FFLVSKLTVD KSRWQQGNVF SCSVMHEALH NHYTQKSLSL SPGK |

The polypeptide sequence of the ActRIIB-Fc fusion protein is provided in Example 1 as SEQ ID NOs: 401-402.

The ActRIIA-Fc and ActRIIB-Fc proteins of SEQ ID NO: 452 and SEQ ID NO: 402, respectively, may be co-expressed and purified from a CHO cell line, to give rise to a variant heteromeric complex comprising ActRIIA-Fc:ActRIIB-Fc.

Purification of various ActRIIA-Fc:ActRIIB-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 31. Generation of an ActRIIA-Fc:BMPRII-Fc Heterodimer

A soluble ActRIIA-Fc:BMPRII-Fc heteromeric complex can be generated comprising the extracellular domains of human ActRIIA and human BMPRII, which are each fused to an Fc domain with a linker positioned between the extracellular domain and the Fc domain. The individual constructs are referred to as ActRIIA-Fc and BMPRII-Fc fusion proteins, respectively.

Formation of heteromeric ActRIIA-Fc:BMPRII-Fc may be guided by approaches similar to those described in Example 30.

### Electrostatic Approach

In a first approach, the polypeptide sequence of the ActRIIA-Fc fusion protein and a nucleic acid sequence encoding it are provided in Example 14 as SEQ ID NOs: 118-120.

The polypeptide sequence of the complementary BMPRII-Fc fusion protein (SEQ ID NO: 155) employs the TPA leader and is as follows:

| (SEQ ID NO: 155) | |
|---|---|
| 1 | MDAMKRGLCC VLLLCGAVFV SPGASQNQER LCAFKDPYQQ DLGIGESRIS |
| 51 | HENGTILCSK GSTCYGLWEK SKGDINLVKQ GCWSHIGDPQ ECHYEECVVT |
| 101 | TTPPSIQNGT YRFCCCSTDL CNVNFTENFP PPDTTPLSPP HSFNRDETGG |
| 151 | GTHTCPPCPA PELLGGPSVF LFPPKPKDTL MISRTPEVTC VVVDVSHEDP |
| 201 | EVKFNWYVDG VEVHNAKTKP REEQYNSTYR VVSVLTVLHQ DWLNGKEYKC |
| 251 | KVSNKALPAP IEKTISKAKG QPREPQVYTL PPSREEMTKN QVSLTCLVKG |
| 301 | FYPSDIAVEW ESNGQPENNY DTTPPVLDSD GSFFLYSDLT VDKSRWQQGN |
| 351 | VFSCSVMHEA LHNHYTQKSL SLSPGK |

The leader and linker sequences are underlined. To promote formation of the ActRIIA-Fc:BMPRII-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 155 may optionally be provided with the lysine removed from the C-terminus. The mature BMPRII-Fc fusion protein sequence is as follows (SEQ ID NO: 156) and may optionally be provided with the lysine removed from the C-terminus.

| (SEQ ID NO: 156) | |
|---|---|
| 1 | SQNQERLCAF KDPYQQDLGI GESRISHENG TILCSKGSTC YGLWEKSKGD |
| 51 | INLVKQGCWS HIGDPQECHY EECVVTTTPP SIQNGTYRFC CCSTDLCNVN |
| 101 | FTENFPPPDT TPLSPPHSFN RDETGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVYTLPPSR EEMTKNQVSL TCLVKGFYPS DIAVEWESNG QPENNYDTTP |
| 301 | PVLDSDGSFF LYSDLTVDKS RWQQGNVFSC SVMHEALHNH YTQKSLSLSP |
| 351 | GK |

The ActRIIA-Fc and BMPRII-Fc fusion proteins of SEQ ID NO: 120 and SEQ ID NO: 156, respectively, may be co-expressed and purified from a CHO cell line to give rise to a heteromeric complex comprising ActRIIA-Fc:BMPRII-Fc.

### Reverse Electrostatic Approach

In a reverse approach, receptor extracellular domains are linked to the opposite member of the Fc interaction pair compared to the first approach described above. In this example, the alternative polypeptide sequence of the ActRIIA-Fc fusion protein is provided in Example 30 as SEQ ID NOs: 151-152.

The polypeptide sequence of the BMPRII-Fc fusion protein and a nucleic acid sequence encoding it are provided in Example 16 as SEQ ID NOs: 121-123.

The ActRIIA-Fc and BMPRII-Fc fusion proteins of SEQ ID NO: 152 and SEQ ID NO: 123, respectively, may be co-expressed and purified from a CHO cell line to give rise to a variant heteromeric complex comprising ActRIIA-Fc:BMPRII-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ActRIIA-Fc fusion protein is provided in Example 14 as SEQ ID NOs 409-410.

The complementary form of BMPRII-Fc fusion polypeptide (SEQ ID NO: 455) is as follows:

| (SEQ ID NO: 455) | |
|---|---|
| 1 | MDAMKRGLCC VLLLCGAVFV SPGASQNQER LCAFKDPYQQ DLGIGESRIS |
| 51 | HENGTILCSK GSTCYGLWEK SKGDINLVKQ GCWSHIGDPQ ECHYEECVVT |
| 101 | TTPPSIQNGT YRFCCCSTDL CNVNFTENFP PPDTTPLSPP HSFNRDETGG |
| 151 | GTHTCPPCPA PELLGGPSVF LFPPKPKDTL MISRTPEVTC VVVDVSHEDP |
| 201 | EVKFNWYVDG VEVHNAKTKP REEQYNSTYR VVSVLTVLHQ DWLNGKEYKC |
| 251 | KVSNKALPAP IEKTISKAKG QPREPQVCTL PPSREEMTKN QVSLSCAVKG |
| 301 | FYPSDIAVEW ESNGQPENNY KTTPPVLDSD GSFFLVSKLT VDKSRWQQGN |
| 351 | VFSCSVMHEA LHNHYTQKSL SLSPGK |

The leader and linker sequences are underlined. To guide heterodimer formation with the ActRIIA-Fc fusion polypeptide of SEQ ID NOs: 409-410, four amino acid substitutions can be introduced into the Fc domain of the BMPRII-Fc fusion polypeptide as indicated by double underline above. The amino acid sequence of SEQ ID NO: 455 may optionally be provided with a lysine removed from the C-terminus.

The mature BMPRII-Fc fusion protein sequence (SEQ ID NO: 456) is as follows and may optionally be provided with a lysine removed from the C-terminus.

| (SEQ ID NO: 456) | |
|---|---|
| 1 | SQNQERLCAF KDPYQQDLGI GESRISHENG TILCSKGSTC YGLWEKSKGD |
| 51 | INLVKQGCWS HIGDPQECHY EECVVTTTPP SIQNGTYRFC CCSTDLCNVN |
| 101 | FTENFPPPDT TPLSPPHSFN RDETGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVCTLPPSR EEMTKNQVSL SCAVKGFYPS DIAVEWESNG QPENNYKTTP |
| 301 | PVLDSDGSFF LVSKLTVDKS RWQQGNVFSC SVMHEALHNH YTQKSLSLSP |
| 351 | GK |

The ActRIIA-Fc and BMPRII-Fc proteins of SEQ ID NO: 410 and SEQ ID NO: 456, respectively, may be co-expressed and purified from a CHO cell line, to give rise to a variant heteromeric complex comprising ActRIIA-Fc:BMPRII-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, the alternative polypeptide sequence of the ActRIIA-Fc fusion protein is provided in Example 30 as SEQ ID NOs: 451-452.

The polypeptide sequence of the BMPRII-Fc fusion protein is provided in Example 16 as SEQ ID NOs: 411-412.

The ActRIIA-Fc and BMPRII-Fc proteins of SEQ ID NO: 452 and SEQ ID NO: 412, respectively, may be co-expressed and purified from a CHO cell line, to give rise to a variant heteromeric complex comprising ActRIIA-Fc:BMPRII-Fc.

Purification of various ActRIIA-Fc:BMPRII-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 32. Generation of an ActRIIA-Fc:MISRII-Fc Heterodimer

A soluble ActRIIA-Fc:MISRII-Fc heteromeric complex can be generated comprising the extracellular domains of human ActRIIA and human MISRII, which are each fused to an Fc domain with a linker positioned between the extracellular domain and the Fc domain. The individual constructs are referred to as ActRIIA-Fc and MISRII-Fc fusion proteins, respectively.

Formation of heteromeric ActRIIA-Fc:MISRII-Fc may be guided by approaches similar to those described in Example 30.

### Electrostatic Approach

In a first approach, the polypeptide sequence of the ActRIIA-Fc fusion protein and a nucleic acid sequence encoding it are provided in Example 14 as SEQ ID NOs: 118-120.

The polypeptide sequence of the complementary MISRII-Fc fusion protein (SEQ ID NO: 161) employs the TPA leader and is as follows:

| (SEQ ID NO: 161) | |
|---|---|
| 1 | MDAMKRGLCC VLLLCGAVFV SPGAPPNRRT CVFFEAPGVR GSTKTLGELL |
| 51 | DTGTELPRAI RCLYSRCCFG IWNLTQDRAQ VEMQGCRDSD EPGCESLHCD |
| 101 | PSPRAHPSPG STLFTCSCGT DFCNANYSHL PPPGSPGTPG SQGPQAAPGE |
| 151 | SIWMALTGGG THTCPPCPAP ELLGGPSVFL FPPKPKDTLM ISRTPEVTCV |
| 201 | VVDVSHEDPE VKFNWYVDGV EVHNAKTKPR EEQYNSTYRV VSVLTVLHQD |
| 251 | WLNGKEYKCK VSNKALPAPI EKTISKAKGQ PREPQVYTLP PSREEMTKNQ |
| 301 | VSLTCLVKGF YPSDIAVEWE SNGQPENNYD TTPPVLDSDG SFFLYSDLTV |
| 351 | DKSRWQQGNV FSCSVMHEAL HNHYTQKSLS LSPGK |

The leader and linker sequences are underlined. To promote formation of the ActRIIA-Fc:MISRII-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 161 may optionally be provided with the lysine removed from the C-terminus.

The mature MISRII-Fc fusion protein sequence is as follows (SEQ ID NO: 162) and may optionally be provided with the lysine removed from the C-terminus.

| (SEQ ID NO: 162) | |
|---|---|
| 1 | PPNRRTCVFF EAPGVRGSTK TLGELLDTGT ELPRAIRCLY SRCCFGIWNL |
| 51 | TQDRAQVEMQ GCRDSDEPGC ESLHCDPSPR AHPSPGSTLF TCSCGTDFCN |
| 101 | ANYSHLPPPG SPGTPGSQGP QAAPGESIWM ALTGGGTHTC PPCPAPELLG |
| 151 | GPSVFLFPPK PKDTLMISRT PEVTCVVVDV SHEDPEVKFN WYVDGVEVHN |
| 201 | AKTKPREEQY NSTYRVVSVL TVLHQDWLNG KEYKCKVSNK ALPAPIEKTI |
| 251 | SKAKGQPREP QVYTLPPSRE EMTKNQVSLT CLVKGFYPSD IAVEWESNGQ |
| 301 | PENNYDTTPP VLDSDGSFFL YSDLTVDKSR WQQGNVFSCS VMHEALHNHY |
| 351 | TQKSLSLSPG K |

The ActRIIA-Fc and MISRII-Fc fusion proteins of SEQ ID NO: 120 and SEQ ID NO: 162, respectively, may be co-expressed and purified from a CHO cell line to give rise to a heteromeric complex comprising ActRIIA-Fc:MISRII-Fc.

### Reverse Electrostatic Approach

In a reverse approach, receptor extracellular domains are linked to the opposite member of the Fc interaction pair compared to the first approach described above. In this example, the alternative polypeptide sequence of the ActRIIA-Fc fusion protein is provided in Example 30 as SEQ ID NOs: 151 and 152.

The polypeptide sequence of the MISRII-Fc fusion protein is provided in Example 29 as SEQ ID NOs: 133 and 135.

The ActRIIA-Fc and MISRII-Fc fusion proteins of SEQ ID NO: 152 and SEQ ID NO: 135, respectively, may be co-expressed and purified from a CHO cell line to give rise to a variant heteromeric complex comprising ActRIIA-Fc:MISRII-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ActRIIA-Fc fusion protein is provided in Example 14 as SEQ ID NOs 409-410.

The complementary form of MISRII-Fc fusion polypeptide (SEQ ID NO: 457) is as follows:

| (SEQ ID NO: 457) | |
|---|---|
| 1 | MDAMKRGLCC VLLLCGAVFV SPGAPPNRRT CVFFEAPGVR GSTKTLGELL |
| 51 | DTGTELPRAI RCLYSRCCFG IWNLTQDRAQ VEMQGCRDSD EPGCESLHCD |
| 101 | PSPRAHPSPG STLFTCSCGT DFCNANYSHL PPPGSPGTPG SQGPQAAPGE |
| 151 | SIWMALTGGG THTCPPCPAP ELLGGPSVFL FPPKPKDTLM ISRTPEVTCV |
| 201 | VVDVSHEDPE VKFNWYVDGV EVHNAKTKPR EEQYNSTYRV VSVLTVLHQD |
| 251 | WLNGKEYKCK VSNKALPAPI EKTISKAKGQ PREPQVCTLP PSREEMTKNQ |
| 301 | VSLSCAVKGF YPSDIAVEWE SNGQPENNYK TTPPVLDSDG SFFLVSKLTV |
| 351 | DKSRWQQGNV FSCSVMHEAL HNHYTQKSLS LSPGK |

The leader and linker sequences are underlined. To guide heterodimer formation with the ActRIIA-Fc fusion polypeptide of SEQ ID NOs: 409-410, four amino acid substitutions can be introduced into the Fc domain of the MISRII-Fc fusion polypeptide as indicated by double underline above. The amino acid sequence of SEQ ID NO: 457 may optionally be provided with a lysine removed from the C-terminus.

The mature MISRII-Fc fusion protein sequence (SEQ ID NO: 458) is as follows and may optionally be provided with a lysine removed from the C-terminus.

| (SEQ ID NO: 458) | |
|---|---|
| 1 | PPNRRTCVFF EAPGVRGSTK TLGELLDTGT ELPRAIRCLY SRCCFGIWNL |
| 51 | TQDRAQVEMQ GCRDSDEPGC ESLHCDPSPR AHPSPGSTLF TCSCGTDFCN |
| 101 | ANYSHLPPPG SPGTPGSQGP QAAPGESIWM ALTGGGTHTC PPCPAPELLG |
| 151 | GPSVFLFPPK PKDTLMISRT PEVTCVVVDV SHEDPEVKFN WYVDGVEVHN |
| 201 | AKTKPREEQY NSTYRVVSVL TVLHQDWLNG KEYKCKVSNK ALPAPIEKTI |
| 251 | SKAKGQPREP QVCTLPPSRE EMTKNQVSLS CAVKGFYPSD IAVEWESNGQ |
| 301 | PENNYKTTPP VLDSDGSFFL VSKLTVDKSR WQQGNVFSCS VMHEALHNHY |
| 351 | TQKSLSLSPG K |

The ActRIIA-Fc and MISRII-Fc proteins of SEQ ID NO: 410 and SEQ ID NO: 458, respectively, may be co-expressed and purified from a CHO cell line, to give rise to a variant heteromeric complex comprising ActRIIA-Fc:MISRII-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, the alternative polypeptide sequence of the ActRIIA-Fc fusion protein is provided in Example 30 as SEQ ID NOs: 451-452.

The polypeptide sequence of the MISRII-Fc fusion protein is provided in Example 29 as SEQ ID NOs: 419-420.

The ActRIIA-Fc and MISRII-Fc proteins of SEQ ID NO: 452 and SEQ ID NO: 420, respectively, may be co-expressed and purified from a CHO cell line, to give rise to a variant heteromeric complex comprising ActRIIA-Fc:MISRII-Fc.

Purification of various ActRIIA-Fc:MISRII-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 33. Generation of an ActRIIA-Fc:TGFβRII_{SHORT}-Fc Heterodimer

A soluble ActRIIA-Fc:TGFβRII_{SHORT}-Fc heteromeric complex can be generated comprising the extracellular domains of human ActRIIA and human TGFβRII_{SHORT}, which are each fused to an Fc domain with a linker positioned between the extracellular domain and the Fc domain. The individual constructs are referred to as ActRIIA-Fc and TGFβRII_{SHORT}-Fc fusion proteins, respectively.

Formation of heteromeric ActRIIA-Fc:TGFβRII_{SHORT}-Fc may be guided by approaches similar to those described in Example 30.

### Electrostatic Approach

In a first approach, the polypeptide sequence of the ActRIIA-Fc fusion protein and a nucleic acid sequence encoding it are provided in Example 14 as SEQ ID NOs: 118-120.

The polypeptide sequence of the complementary TGFβRII_{SHORT}-Fc fusion protein (SEQ ID NO: 157) employs the TPA leader and is as follows:

| (SEQ ID NO: 157) | |
|---|---|
| 1 | MDAMKRGLCC VLLLCGAVFV SPGATIPPHV QKSVNNDMIV TDNNGAVKFP |
| 51 | QLCKFCDVRF STCDNQKSCM SNCSITSICE KPQEVCVAVW RKNDENITLE |
| 101 | TVCHDPKLPY HDFILEDAAS PKCIMKEKKK PGETFFMCSC SSDECNDNII |
| 151 | FSEEYNTSNP DTGGGTHTCP PCPAPELLGG PSVFLFPPKP KDTLMISRTP |
| 201 | EVTCVVVDVS HEDPEVKFNW YVDGVEVHNA KTKPREEQYN STYRVVSVLT |
| 251 | VLHQDWLNGK EYKCKVSNKA LPAPIEKTIS KAKGQPREPQ VYTLPPSREE |
| 301 | MTKNQVSLTC LVKGFYPSDI AVEWESNGQP ENNYDTTPPV LDSDGSFFLY |
| 351 | SDLTVDKSRW QQGNVFSCSV MHEALHNHYT QKSLSLSPGK |

The leader and linker sequences are underlined. To promote formation of the ActRIIA-Fc: TGFβRII_{SHORT}-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 157 may optionally be provided with the lysine removed from the C-terminus.

The mature TGFβRII_{SHORT}-Fc fusion protein sequence is as follows (SEQ ID NO: 158) and may optionally be provided with the lysine removed from the C-terminus.

| (SEQ ID NO: 158) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE YNTSNPDTGG GTHTCPPCPA |
| 151 | PELLGGPSVF LFPPKPKDTL MISRTPEVTC VVVDVSHEDP EVKFNWYVDG |
| 201 | VEVHNAKTKP REEQYNSTYR VVSVLTVLHQ DWLNGKEYKC KVSNKALPAP |
| 251 | IEKTISKAKG QPREPQVYTL PPSREEMTKN QVSLTCLVKG FYPSDIAVEW |
| 301 | ESNGQPENNY DTTPPVLDSD GSFFLYSDLT VDKSRWQQGN VFSCSVMHEA |
| 351 | LHNHYTQKSL SLSPGK |

The ActRIIA-Fc and TGFβRII_{SHORT}-Fc fusion proteins of SEQ ID NO: 120 and SEQ ID NO: 158, respectively, may be co-expressed and purified from a CHO cell line to give rise to a heteromeric complex comprising ActRIIA-Fc:TGFβRII_{SHORT}-Fc.

### Reverse Electrostatic Approach

In a reverse approach, receptor extracellular domains are linked to the opposite member of the Fc interaction pair compared to the first approach described above. In this example, the alternative polypeptide sequence of the ActRIIA-Fc fusion protein is provided in Example 30 as SEQ ID NOs: 151-152.

The polypeptide sequence of the TGFβRII_{SHORT}-Fc fusion protein and a nucleic acid sequence encoding it are provided in Example 24 as SEQ ID NOs: 127-129.

The ActRIIA-Fc and TGFβRII_{SHORT}-Fc fusion proteins of SEQ ID NO: 152 and SEQ ID NO: 129, respectively, may be co-expressed and purified from a CHO cell line to give rise to a variant heteromeric complex comprising ActRIIA-Fc:TGFβRII_{SHORT}-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ActRIIA-Fc fusion protein is provided in Example 14 as SEQ ID NOs 409-410. The complementary form of TGFβRII_{SHORT}-Fc fusion polypeptide (SEQ ID NO: 459) is as follows:

| (SEQ ID NO: 459) | |
|---|---|
| 1 | MDAMKRGLCC VLLLCGAVFV SPGATIPPHV QKSVNNDMIV TDNNGAVKFP |
| 51 | QLCKFCDVRF STCDNQKSCM SNCSITSICE KPQEVCVAVW RKNDENITLE |
| 101 | TVCHDPKLPY HDFILEDAAS PKCIMKEKKK PGETFFMCSC SSDECNDNII |
| 151 | FSEEYNTSNP DTGGGTHTCP PCPAPELLGG PSVFLFPPKP KDTLMISRTP |
| 201 | EVTCVVVDVS HEDPEVKFNW YVDGVEVHNA KTKPREEQYN STYRVVSVLT |
| 251 | VLHQDWLNGK EYKCKVSNKA LPAPIEKTIS KAKGQPREPQ VCTLPPSREE |
| 301 | MTKNQVSLSC AVKGFYPSDI AVEWESNGQP ENNYKTTPPV LDSDGSFFLV |
| 351 | SKLTVDKSRW QQGNVFSCSV MHEALHNHYT QKSLSLSPGK |

The leader and linker sequences are underlined. To guide heterodimer formation with the ActRIIA-Fc fusion polypeptide of SEQ ID NOs: 409-410, four amino acid substitutions can be introduced into the Fc domain of the TGFβRII_{SHORT}-Fc fusion polypeptide as indicated by double underline above. The amino acid sequence of SEQ ID NO: 459 may optionally be provided with a lysine removed from the C-terminus.

The mature TGFβRII_{SHORT}-Fc fusion protein sequence (SEQ ID NO: 460) is as follows and may optionally be provided with a lysine removed from the C-terminus.

| (SEQ ID NO: 460) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE YNTSNPDTGG GTHTCPPCPA |
| 151 | PELLGGPSVF LFPPKPKDTL MISRTPEVTC VVVDVSHEDP EVKFNWYVDG |
| 201 | VEVHNAKTKP REEQYNSTYR VVSVLTVLHQ DWLNGKEYKC KVSNKALPAP |
| 251 | IEKTISKAKG QPREPQVCTL PPSREEMTKN QVSLSCAVKG FYPSDIAVEW |
| 301 | ESNGQPENNY KTTPPVLDSD GSFFLVSKLT VDKSRWQQGN VFSCSVMHEA |
| 351 | LHNHYTQKSL SLSPGK |

The ActRIIA-Fc and TGFβRII_{SHORT}-Fc proteins of SEQ ID NO: 410 and SEQ ID NO: 460, respectively, may be co-expressed and purified from a CHO cell line, to give rise to a variant heteromeric complex comprising ActRIIA-Fc:TGFβRII_{SHORT}-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, the alternative polypeptide sequence of the ActRIIA-Fc fusion protein is provided in Example 30 as SEQ ID NOs: 451-452.

The polypeptide sequence of the TGFβRII_{SHORT}-Fc fusion protein is provided in Example 24 as SEQ ID NOs: 415-416.

The ActRIIA-Fc and TGFβRII_{SHORT}-Fc proteins of SEQ ID NO: 452 and SEQ ID NO: 416, respectively, may be co-expressed and purified from a CHO cell line, to give rise to a variant heteromeric complex comprising ActRIIA-Fc:TGFβRII_{SHORT}-Fc.

Purification of various ActRIIA-Fc:TGFβRII_{SHORT}-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 34. Generation of an ActRIIA-Fc:TGFβRII_{LONG}-Fc Heterodimer

A soluble ActRIIA-Fc:TGFβRII_{LONG}-Fc heteromeric complex can be generated comprising the extracellular domains of human ActRIIA and human TGFβRII_{LONG}, which are each fused to an Fc domain with a linker positioned between the extracellular domain and the Fc domain. The individual constructs are referred to as ActRIIA-Fc and TGFβRII_{LONG}-Fc fusion proteins, respectively.

Formation of heteromeric ActRIIA-Fc:TGFβRII_{LONG}-Fc may be guided by approaches similar to those described in Example 30.

### Electrostatic Approach

In a first approach, the polypeptide sequence of the ActRIIA-Fc fusion protein and a nucleic acid sequence encoding it are provided in Example 14 as SEQ ID NOs: 118-120.

The polypeptide sequence of the complementary TGFβRII_{LONG}-Fc fusion protein (SEQ ID NO: 159) employs the TPA leader and is as follows:

| (SEQ ID NO: 159) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG THTCPPCPAP |
| 201 | ELLGGPSVFL FPPKPKDTLM ISRTPEVTCV VVDVSHEDPE VKFNWYVDGV |
| 251 | EVHNAKTKPR EEQYNSTYRV VSVLTVLHQD WLNGKEYKCK VSNKALPAPI |
| 301 | EKTISKAKGQ PREPQVYTLP PSREEMTKNQ VSLTCLVKGF YPSDIAVEWE |
| 351 | SNGQPENNYD TTPPVLDSDG SFFLYSDLTV DKSRWQQGNV FSCSVMHEAL |
| 401 | HNHYTQKSLS LSPGK |

The leader and linker sequences are underlined. To promote formation of the ActRIIA-Fc:TGFβRII_{LONG}-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 159 may optionally be provided with the lysine removed from the C-terminus.

The mature TGFβRII_{LONG}-Fc fusion protein sequence is as follows (SEQ ID NO: 160) and may optionally be provided with the lysine removed from the C-terminus.

| (SEQ ID NO: 160) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PDTGGGTHTC PPCPAPELLG GPSVFLFPPK PKDTLMISRT |
| 201 | PEVTCVVVDV SHEDPEVKFN WYVDGVEVHN AKTKPREEQY NSTYRVVSVL |
| 251 | TVLHQDWLNG KEYKCKVSNK ALPAPIEKTI SKAKGQPREP QVYTLPPSRE |
| 301 | EMTKNQVSLT CLVKGFYPSD IAVEWESNGQ PENNYDTTPP VLDSDGSFFL |
| 351 | YSDLTVDKSR WQQGNVFSCS VMHEALHNHY TQKSLSLSPG K |

The ActRIIA-Fc and TGFβRII_{LONG}-Fc fusion proteins of SEQ ID NO: 120 and SEQ ID NO: 160, respectively, may be co-expressed and purified from a CHO cell line to give rise to a heteromeric complex comprising ActRIIA-Fc:TGFβRII_{LONG}-Fc.

### Reverse Electrostatic Approach

In a reverse approach, receptor extracellular domains are linked to the opposite member of the Fc interaction pair compared to the first approach described above. In this example, the alternative polypeptide sequence of the ActRIIA-Fc fusion protein is provided in Example 30 as SEQ ID NOs: 151-152.

The polypeptide sequence of the TGFβRII_{LONG}-Fc fusion protein and a nucleic acid sequence encoding it are provided in Example 24 as SEQ ID NOs: 130-132.

The ActRIIA-Fc and TGFβRII_{LONG}-Fc fusion proteins of SEQ ID NO: 152 and SEQ ID NO: 132, respectively, may be co-expressed and purified from a CHO cell line to give rise to a variant heteromeric complex comprising ActRIIA-Fc:TGFβRII_{LONG}-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ActRIIA-Fc fusion protein is provided in Example 14 as SEQ ID NOs 409-410. The complementary form of TGFβRII_{LONG}-Fc fusion polypeptide (SEQ ID NO: 461) is as follows:

| (SEQ ID NO: 461) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG THTCPPCPAP |
| 201 | ELLGGPSVFL FPPKPKDTLM ISRTPEVTCV VVDVSHEDPE VKFNWYVDGV |
| 251 | EVHNAKTKPR EEQYNSTYRV VSVLTVLHQD WLNGKEYKCK VSNKALPAPI |
| 301 | EKTISKAKGQ PREPQVCTLP PSREEMTKNQ VSLSCAVKGF YPSDIAVEWE |
| 351 | SNGQPENNYK TTPPVLDSDG SFFLVSKLTV DKSRWQQGNV FSCSVMHEAL |
| 401 | HNHYTQKSLS LSPGK |

The leader and linker sequences are underlined. To guide heterodimer formation with the ActRIIA-Fc fusion polypeptide of SEQ ID NOs: 409-410, four amino acid substitutions can be introduced into the Fc domain of the ActRIIB-Fc fusion polypeptide as indicated by double underline above. The amino acid sequence of SEQ ID NO: 461 may optionally be provided with the lysine removed from the C-terminus.

The mature TGFβRII_{LONG}-Fc fusion protein sequence (SEQ ID NO: 462) is as follows and may optionally be provided with the lysine removed from the C-terminus.

| (SEQ ID NO: 462) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PDTGGGTHTC PPCPAPELLG GPSVFLFPPK PKDTLMISRT |
| 201 | PEVTCVVVDV SHEDPEVKFN WYVDGVEVHN AKTKPREEQY NSTYRVVSVL |
| 251 | TVLHQDWLNG KEYKCKVSNK ALPAPIEKTI SKAKGQPREP QVCTLPPSRE |
| 301 | EMTKNQVSLS CAVKGFYPSD IAVEWESNGQ PENNYKTTPP VLDSDGSFFL |
| 351 | VSKLTVDKSR WQQGNVFSCS VMHEALHNHY TQKSLSLSPG K |

The ActRIIA-Fc and TGFβRII_{LONG}-Fc proteins of SEQ ID NO: 410 and SEQ ID NO: 462, respectively, may be co-expressed and purified from a CHO cell line, to give rise to a variant heteromeric complex comprising ActRIIA-Fc: TGFβRII_{LONG}-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, the alternative polypeptide sequence of the ActRIIA-Fc fusion protein is provided in Example 30 as SEQ ID NOs: 451-452.

The polypeptide sequence of the TGFβRII_{LONG}-Fc fusion protein is provided in Example 24 as SEQ ID NOs: 417-418.

The ActRIIA-Fc and TGFβRII_{LONG}-Fc proteins of SEQ ID NO: 452 and SEQ ID NO: 418, respectively, may be co-expressed and purified from a CHO cell line, to give rise to a variant heteromeric complex comprising ActRIIA-Fc:TGFβRII_{LONG}-Fc.

Purification of various ActRIIA-Fc:TGFβRII_{LONG}-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 35. Generation of Additional Heterodimers Comprising Two Type II Receptors

Formation of additional heterdimeric protein complexes comprising extracellular domains of two type II receptors may be guided by approaches similar to those described in Example 30. Amino acid SEQ ID NOs for these variant heterodimers are disclosed in the following table, which for completeness also includes the heterodimers already discussed that comprise two type II receptors (Examples 30-34). As disclosed in Examples 30-34, the C-terminal lysine of each receptor-Fc fusion protein may optionally be included or omitted.

In each case, a soluble heteromeric complex can be generated comprising the extracellular domains (ECD) of two different type II receptors, which are each fused to an Fc domain with a linker positioned between the extracellular domain and the Fc domain. In each case, the fusion proteins may be co-expressed and purified from a CHO cell line to give rise to a variant heteromeric protein complex. In each case, purification of various heteromeric complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

TABLE
“Type II:Type II Receptor Heterodimers”
Amino Acid SEQ ID NOs for Type II:Type II Receptor Heterodimers

| | | | | Amino Acid | |
|---|---|---|---|---|---|
| Heteromeric | | ECD | | SEQ ID NO | |
| Fusion Protein | | Correspondence | Receptor-Fc | With | |
| Complex | Fc Pair Type | to Fc Pair | Fusion Protein | Leader | Mature |
| ActRIIA-Fc:ActRIIB-Fc | Electrostatic | A | ActRIIA-Fc | 118 | 120 |
| | | | ActRIIB-Fc | 153 | 154 |
| | | B | ActRIIA-Fc | 151 | 152 |
| | | | ActRIIB-Fc | 100 | 102 |
| | Hydrophobic | A | ActRIIA-Fc | 409 | 410 |
| | | | ActRIIB-Fc | 453 | 454 |
| | | B | ActRIIA-Fc | 451 | 452 |
| | | | ActRIIB-Fc | 401 | 402 |
| ActRIIA-Fc:BMPRII-Fc | Electrostatic | A | ActRIIA-Fc | 118 | 120 |
| | | | BMPRII-Fc | 155 | 156 |
| | | B | ActRIIA-Fc | 151 | 152 |
| | | | BMPRII-Fc | 121 | 123 |
| | Hydrophobic | A | ActRIIA-Fc | 409 | 410 |
| | | | BMPRII-Fc | 455 | 456 |
| | | B | ActRIIA-Fc | 451 | 452 |
| | | | BMPRII-Fc | 411 | 412 |
| ActRIIA-Fc:MISRII-Fc | Electrostatic | A | ActRIIA-Fc | 118 | 120 |
| | | | MISRII-Fc | 161 | 162 |
| | | B | ActRIIA-Fc | 151 | 152 |
| | | | MISRII-Fc | 133 | 135 |
| | Hydrophobic | A | ActRIIA-Fc | 409 | 410 |
| | | | MISRII-Fc | 457 | 458 |
| | | B | ActRIIA-Fc | 451 | 452 |
| | | | MISRII-Fc | 419 | 420 |
| ActRIIA-Fc:TGFβRII_{SHORT}-Fc | Electrostatic | A | ActRIIA-Fc | 118 | 120 |
| | | | TGFβRII_{SHORT}-Fc | 157 | 158 |
| | | B | ActRIIA-Fc | 151 | 152 |
| | | | TGFβRII_{SHORT}-Fc | 127 | 129 |
| | Hydrophobic | A | ActRIIA-Fc | 409 | 410 |
| | | | TGFβRII_{SHORT}-Fc | 459 | 460 |
| | | B | ActRIIA-Fc | 451 | 452 |
| | | | TGFβRII_{SHORT}-Fc | 415 | 416 |
| ActRIIA-Fc:TGFβRII_{LONG}-Fc | Electrostatic | A | ActRIIA-Fc | 118 | 120 |
| | | | TGFβRII_{LONG}-Fc | 159 | 160 |
| | | B | ActRIIA-Fc | 151 | 152 |
| | | | TGFβRII_{LONG}-Fc | 130 | 132 |
| | Hydrophobic | A | ActRIIA-Fc | 409 | 410 |
| | | | TGFβRII_{LONG}-Fc | 461 | 462 |
| | | B | ActRIIA-Fc | 451 | 452 |
| | | | TGFβRII_{LONG}-Fc | 417 | 418 |
| ActRIIB-Fc:BMPRII-Fc | Electrostatic | A | ActRIIB-Fc | 100 | 102 |
| | | | BMPRII-Fc | 155 | 156 |
| | | B | ActRIIB-Fc | 153 | 154 |
| | | | BMPRII-Fc | 121 | 123 |
| | Hydrophobic | A | ActRIIB-Fc | 401 | 402 |
| | | | BMPRII-Fc | 455 | 456 |
| | | B | ActRIIB-Fc | 453 | 454 |
| | | | BMPRII-Fc | 411 | 412 |
| ActRIIB-Fc:MISRII-Fc | Electrostatic | A | ActRIIB-Fc | 100 | 102 |
| | | | MISRII-Fc | 161 | 162 |
| | | B | ActRIIB-Fc | 153 | 154 |
| | | | MISRII-Fc | 133 | 135 |
| | Hydrophobic | A | ActRIIB-Fc | 401 | 402 |
| | | | MISRII-Fc | 457 | 458 |
| | | B | ActRIIB-Fc | 453 | 454 |
| | | | MISRII-Fc | 419 | 420 |
| ActRIIB-Fc:TGFβRII_{SHORT}-Fc | Electrostatic | A | ActRIIB-Fc | 100 | 102 |
| | | | TGFβRII_{SHORT}-Fc | 157 | 158 |
| | | B | ActRIIB-Fc | 153 | 154 |
| | | | TGFβRII_{SHORT}-Fc | 127 | 129 |
| | Hydrophobic | A | ActRIIB-Fc | 401 | 402 |
| | | | TGFβRII_{SHORT}-Fc | 459 | 460 |
| | | B | ActRIIB-Fc | 453 | 454 |
| | | | TGFβRII_{SHORT}-Fc | 415 | 416 |
| ActRIIB-Fc:TGFβRII_{LONG}-Fc | Electrostatic | A | ActRIIB-Fc | 100 | 102 |
| | | | TGFβRII_{LONG}-Fc | 159 | 160 |
| | | B | ActRIIB-Fc | 153 | 154 |
| | | | TGFβRII_{LONG}-Fc | 130 | 132 |
| | Hydrophobic | A | ActRIIB-Fc | 401 | 402 |
| | | | TGFβRII_{LONG}-Fc | 461 | 462 |
| | | B | ActRIIB-Fc | 453 | 454 |
| | | | TGFβRII_{LONG}-Fc | 417 | 418 |
| BMPRII-Fc:MISRII-Fc | Electrostatic | A | BMPRII-Fc | 121 | 123 |
| | | | MISRII-Fc | 161 | 162 |
| | | B | BMPRII-Fc | 155 | 156 |
| | | | MISRII-Fc | 133 | 135 |
| | Hydrophobic | A | BMPRII-Fc | 411 | 412 |
| | | | MISRII-Fc | 457 | 458 |
| | | B | BMPRII-Fc | 455 | 456 |
| | | | MISRII-Fc | 419 | 420 |
| BMPRII-Fc:TGFβRII_{SHORT}-Fc | Electrostatic | A | BMPRII-Fc | 121 | 123 |
| | | | TGFβRII_{SHORT}-Fc | 157 | 158 |
| | | B | BMPRII-Fc | 155 | 156 |
| | | | TGFβRII_{SHORT}-Fc | 127 | 129 |
| | Hydrophobic | A | BMPRII-Fc | 411 | 412 |
| | | | TGFβRII_{SHORT}-Fc | 459 | 460 |
| | | B | BMPRII-Fc | 455 | 456 |
| | | | TGFβRII_{SHORT}-Fc | 415 | 416 |
| BMPRII-Fc:TGFβRII_{LONG}-Fc | Electrostatic | A | BMPRII-Fc | 121 | 123 |
| | | | TGFβRII_{LONG}-Fc | 159 | 160 |
| | | B | BMPRII-Fc | 155 | 156 |
| | | | TGFβRII_{LONG}-Fc | 130 | 132 |
| | Hydrophobic | A | BMPRII-Fc | 411 | 412 |
| | | | TGFβRII_{LONG}-Fc | 461 | 462 |
| | | B | BMPRII-Fc | 455 | 456 |
| | | | TGFβRII_{LONG}-Fc | 417 | 418 |
| MISRII-Fc:TGFβRII_{SHORT}-Fc | Electrostatic | A | MISRII-Fc | 133 | 135 |
| | | | TGFβRII_{SHORT}-Fc | 157 | 158 |
| | | B | MISRII-Fc | 161 | 162 |
| | | | TGFβRII_{SHORT}-Fc | 127 | 129 |
| | Hydrophobic | A | MISRII-Fc | 419 | 420 |
| | | | TGFβRII_{SHORT}-Fc | 459 | 460 |
| | | B | MISRII-Fc | 457 | 458 |
| | | | TGFβRII_{SHORT}-Fc | 415 | 416 |
| MISRII-Fc:TGFβRII_{LONG}-Fc | Electrostatic | A | MISRII-Fc | 133 | 135 |
| | | | TGFβRII_{LONG}-Fc | 159 | 160 |
| | | B | MISRII-Fc | 161 | 162 |
| | | | TGFβRII_{LONG}-Fc | 130 | 132 |
| | Hydrophobic | A | MISRII-Fc | 419 | 420 |
| | | | TGFβRII_{LONG}-Fc | 461 | 462 |
| | | B | MISRII-Fc | 457 | 458 |
| | | | TGFβRII_{LONG}-Fc | 417 | 418 |
| TGFβRII_{SHORT}-Fc:TGFβRII_{LONG}-Fc | Electrostatic | A | TGFβRII_{SHORT}-Fc | 127 | 129 |
| | | | TGFβRII_{LONG}-Fc | 159 | 160 |
| | | B | TGFβRII_{SHORT}-Fc | 157 | 158 |
| | | | TGFβRII_{LONG}-Fc | 130 | 132 |
| | Hydrophobic | A | TGFβRII_{SHORT}-Fc | 415 | 416 |
| | | | TGFβRII_{LONG}-Fc | 461 | 462 |
| | | B | TGFβRII_{SHORT}-Fc | 459 | 460 |
| | | | TGFβRII_{LONG}-Fc | 417 | 418 |

### Example 36. Generation of an ALK1-Fc:ALK2-Fc Heterodimer

A soluble ALK1-Fc:ALK2-Fc heteromeric complex can be generated comprising the extracellular domains of human ALK1 and human ALK2, which are each fused to an Fc domain with a linker positioned between the extracellular domain and the Fc domain. The individual constructs are referred to as ALK1-Fc and ALK2-Fc fusion proteins, respectively.

Formation of heteromeric ALK1-Fc:ALK2-Fc may be guided by approaches similar to those described in Example 1.

### Electrostatic Approach

In a first approach, the polypeptide sequence of the ALK1-Fc fusion protein and a nucleic acid sequence encoding it are provided above in Example 16 as SEQ ID NOs: 124-126.

The polypeptide sequence of the complementary ALK2-Fc fusion protein (SEQ ID NO: 173) employs the TPA leader and is as follows:

| (SEQ ID NO: 173) | |
|---|---|
| 1 | MDAMKRGLCC VLLLCGAVFV SPGAMEDEKP KVNPKLYMCV CEGLSCGNED |
| 51 | HCEGQQCFSS LSINDGFHVY QKGCFQVYEQ GKMTCKTPPS PGQAVECCQG |
| 101 | DWCNRNITAQ LPTKGKSFPG TQNFHLETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVYTL PPSRKEMTKN QVSLTCLVKG FYPSDIAVEW ESNGQPENNY |
| 301 | KTTPPVLKSD GSFFLYSKLT VDKSRWQQGN VFSCSVMHEA LHNHYTQKSL |
| 351 | SLSPG |

The leader and linker sequences are underlined. To promote formation of the ALK1-Fc:ALK2-Fc heterodimer rather than either of the possible homodimeric complexes, two amino acid substitutions (replacing acidic amino acids with lysines) can be introduced into the Fc domain of the fusion protein as indicated by double underline above. The amino acid sequence of SEQ ID NO: 173 may optionally be provided with a lysine added to the C-terminus.

The mature ALK2-Fc fusion protein sequence is as follows (SEQ ID NO: 174) and may optionally be provided with a lysine added to the C-terminus.

| (SEQ ID NO: 174) | |
|---|---|
| 1 | MEDEKPKVNP KLYMCVCEGL SCGNEDHCEG QQCFSSLSIN DGFHVYQKGC |
| 51 | FQVYEQGKMT CKTPPSPGQA VECCQGDWCN RNITAQLPTK GKSFPGTQNF |
| 101 | HLETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVYTLPPSR KEMTKNQVSL |
| 251 | TCLVKGFYPS DIAVEWESNG QPENNYKTTP PVLKSDGSFF LYSKLTVDKS |
| 301 | RWQQGNVFSC SVMHEALHNH YTQKSLSLSP G |

The ALK1-Fc and ALK2-Fc fusion proteins of SEQ ID NO: 126 and SEQ ID NO: 174, respectively, may be co-expressed and purified from a CHO cell line to give rise to a heteromeric complex comprising ALK1-Fc:ALK2-Fc.

### Reverse Electrostatic Approach

In a reverse approach, receptor extracellular domains are both linked to the opposite member of the Fc interaction pair compared to the first approach described above. In this example, an alternative polypeptide sequence of the ALK1-Fc fusion protein (SEQ ID NO: 171) employs the TPA leader and is as follows.

| (SEQ ID NO: 171) | |
|---|---|
| 1 | MDAMKRGLCC VLLLCGAVFV SPGADPVKPS RGPLVTCTCE SPHCKGPTCR |
| 51 | GAWCTVVLVR EEGRHPQEHR GCGNLHRELC RGRPTEFVNH YCCDSHLCNH |
| 101 | NVSLVLEATQ PPSEQPGTDG QLATGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVYTLPPSR KEMTKNQVSL TCLVKGFYPS DIAVEWESNG QPENNYKTTP |
| 301 | PVLKSDGSFF LYSKLTVDKS RWQQGNVFSC SVMHEALHNH YTQKSLSLSP |
| 351 | G |

The leader and linker sequences are underlined. To promote formation of the ALK1-Fc:ALK2-Fc heterodimer rather than either of the possible homodimeric complexes, two amino acid substitutions (replacing acidic amino acids with lysines) can be introduced into the Fc domain of the fusion protein as indicated by double underline above. The amino acid sequence of SEQ ID NO: 171 may optionally be provided with a lysine added to the C-terminus.

The mature ALK1-Fc fusion protein sequence is as follows (SEQ ID NO: 172) and may optionally be provided with a lysine added to the C-terminus.

| (SEQ ID NO: 172) | |
|---|---|
| 1 | DPVKPSRGPL VTCTCESPHC KGPTCRGAWC TVVLVREEGR HPQEHRGCGN |
| 51 | LHRELCRGRP TEFVNHYCCD SHLCNHNVSL VLEATQPPSE QPGTDGQLAT |
| 101 | GGGTHTCPPC PAPELLGGPS VFLFPPKPKD TLMISRTPEV TCVVVDVSHE |
| 151 | DPEVKFNWYV DGVEVHNAKT KPREEQYNST YRVVSVLTVL HQDWLNGKEY |
| 201 | KCKVSNKALP APIEKTISKA KGQPREPQVY TLPPSRKEMT KNQVSLTCLV |
| 251 | KGFYPSDIAV EWESNGQPEN NYKTTPPVLK SDGSFFLYSK LTVDKSRWQQ |
| 301 | GNVFSCSVMH EALHNHYTQK SLSLSPG |

The polypeptide sequence of the ALK2-Fc fusion protein and a nucleic acid sequence encoding it are provided in Example 9 as SEQ ID NOs: 136-138.

The ALK1-Fc and ALK2-Fc fusion proteins of SEQ ID NO: 172 and SEQ ID NO: 138, respectively, may be co-expressed and purified from a CHO cell line to give rise to a variant heteromeric complex comprising ALK1-Fc:ALK2-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ALK1-Fc fusion protein is provided in Example 16 as SEQ ID NOs: 413-414. The complementary form of ALK2-Fc fusion polypeptide (SEQ ID NO: 465) is as follows:

| (SEQ ID NO: 465) | |
|---|---|
| 1 | MDAMKRGLCC VLLLCGAVFV SPGAMEDEKP KVNPKLYMCV CEGLSCGNED |
| 51 | HCEGQQCFSS LSINDGFHVY QKGCFQVYEQ GKMTCKTPPS PGQAVECCQG |
| 101 | DWCNRNITAQ LPTKGKSFPG TQNFHLETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVYTL PPCREEMTKN QVSLWCLVKG FYPSDIAVEW ESNGQPENNY |
| 301 | KTTPPVLDSD GSFFLYSKLT VDKSRWQQGN VFSCSVMHEA LHNHYTQKSL |
| 351 | SLSPGK |

The leader and linker sequences are underlined. To guide heterodimer formation with the ALK1-Fc fusion polypeptide of SEQ ID NOs: 413-414, two amino acid substitutions (replacing a serine with a cysteine and a threonine with a tryptophan) can be introduced into the Fc domain of the ALK2-Fc fusion polypeptide as indicated by double underline above. The amino acid sequence of SEQ ID NO: 465 may optionally be provided with the lysine removed from the C-terminus.

The mature ALK2-Fc fusion protein sequence (SEQ ID NO: 466) is as follows and may optionally be provided with the lysine removed from the C-terminus.

| (SEQ ID NO: 466) | |
|---|---|
| 1 | MEDEKPKVNP KLYMCVCEGL SCGNEDHCEG QQCFSSLSIN DGFHVYQKGC |
| 51 | FQVYEQGKMT CKTPPSPGQA VECCQGDWCN RNITAQLPTK GKSFPGTQNF |
| 101 | HLETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVYTLPPCR EEMTKNQVSL |
| 251 | WCLVKGFYPS DIAVEWESNG QPENNYKTTP PVLDSDGSFF LYSKLTVDKS |
| 301 | RWQQGNVFSC SVMHEALHNH YTQKSLSLSP GK |

The ALK1-Fc and ALK2-Fc proteins of SEQ ID NO: 414 and SEQ ID NO: 466, respectively, may be co-expressed and purified from a CHO cell line, to give rise to a variant heteromeric complex comprising ALK1-Fc:ALK2-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, an alternative polypeptide sequence of the ALK1-Fc fusion protein (SEQ ID NO: 463) employs the TPA leader and is as follows.

| (SEQ ID NO: 463) | |
|---|---|
| 1 | MDAMKRGLCC VLLLCGAVFV SPGADPVKPS RGPLVTCTCE SPHCKGPTCR |
| 51 | GAWCTVVLVR EEGRHPQEHR GCGNLHRELC RGRPTEFVNH YCCDSHLCNH |
| 101 | NVSLVLEATQ PPSEQPGTDG QLATGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVYTLPPCR EEMTKNQVSL WCLVKGFYPS DIAVEWESNG QPENNYKTTP |
| 301 | PVLDSDGSFF LYSKLTVDKS RWQQGNVFSC SVMHEALHNH YTQKSLSLSP |
| 351 | GK |

The leader and linker sequences are underlined. To guide heterodimer formation with the ALK2-Fc fusion polypeptide of SEQ ID NOs 421-422, two amino acid substitutions (replacing a serine with a cysteine and a threonine with a tryptophan) can be introduced into the Fc domain of the ALK1-Fc fusion polypeptide as indicated by double underline above. The amino acid sequence of SEQ ID NO: 463 may optionally be provided with the lysine removed from the C-terminus.

The mature ALK1-Fc fusion protein sequence is as follows (SEQ ID NO: 464) and may optionally be provided with the lysine removed from the C-terminus.

| (SEQ ID NO: 464) | |
|---|---|
| 1 | DPVKPSRGPL VTCTCESPHC KGPTCRGAWC TVVLVREEGR HPQEHRGCGN |
| 51 | LHRELCRGRP TEFVNHYCCD SHLCNHNVSL VLEATQPPSE QPGTDGQLAT |
| 101 | GGGTHTCPPC PAPELLGGPS VFLFPPKPKD TLMISRTPEV TCVVVDVSHE |
| 151 | DPEVKFNWYV DGVEVHNAKT KPREEQYNST YRVVSVLTVL HQDWLNGKEY |
| 201 | KCKVSNKALP APIEKTISKA KGQPREPQVY TLPPCREEMT KNQVSLWCLV |
| 251 | KGFYPSDIAV EWESNGQPEN NYKTTPPVLD SDGSFFLYSK LTVDKSRWQQ |
| 301 | GNVFSCSVMH EALHNHYTQK SLSLSPGK |

The polypeptide sequence of the ALK2-Fc fusion protein is provided in Example 9 as SEQ ID NOs: 421-422.

The ALK1-Fc and ALK2-Fc proteins of SEQ ID NO: 464 and SEQ ID NO: 422, respectively, may be co-expressed and purified from a CHO cell line, to give rise to a variant heteromeric complex comprising ALK1-Fc:ALK2-Fc.

Purification of various ALK1-Fc:ALK2-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 37. Generation of an ALK1-Fc:ALK3-Fc Heterodimer

A soluble ALK1-Fc:ALK3-Fc heteromeric complex can be generated comprising the extracellular domains of human ALK1 and human ALK3, which are each fused to an Fc domain with a linker positioned between the extracellular domain and the Fc domain. The individual constructs are referred to as ALK1-Fc and ALK3-Fc fusion proteins, respectively.

Formation of heteromeric ALK1-Fc:ALK3-Fc may be guided by approaches similar to those described in Example 36.

### Electrostatic Approach

In a first approach, the polypeptide sequence of the ALK1-Fc fusion protein and a nucleic acid sequence encoding it are provided in Example 16 as SEQ ID NOs: 124-126.

The polypeptide sequence of the complementary ALK3-Fc fusion protein (SEQ ID NO: 175) employs the TPA leader and is as follows:

| (SEQ ID NO: 175) | |
|---|---|
| 1 | MDAMKRGLCC VLLLCGAVFV SPGAQNLDSM LHGTGMKSDS DQKKSENGVT |
| 51 | LAPEDTLPFL KCYCSGHCPD DAINNTCITN GHCFAIIEED DQGETTLASG |
| 101 | CMKYEGSDFQ CKDSPKAQLR RTIECCRTNL CNQYLQPTLP PVVIGPFFDG |
| 151 | SIRTGGGTHT CPPCPAPELL GGPSVFLFPP KPKDTLMISR TPEVTCVVVD |
| 201 | VSHEDPEVKF NWYVDGVEVH NAKTKPREEQ YNSTYRVVSV LTVLHQDWLN |
| 251 | GKEYKCKVSN KALPAPIEKT ISKAKGQPRE PQVYTLPPSR KEMTKNQVSL |
| 301 | TCLVKGFYPS DIAVEWESNG QPENNYKTTP PVLKSDGSFF LYSKLTVDKS |
| 351 | RWQQGNVFSC SVMHEALHNH YTQKSLSLSP G |

The leader and linker sequences are underlined. To promote formation of the ALK1-Fc:ALK3-Fc heterodimer rather than either of the possible homodimeric complexes, two amino acid substitutions (replacing acidic amino acids with lysines) can be introduced into the Fc domain of the fusion protein as indicated by double underline above. The amino acid sequence of SEQ ID NO: 175 may optionally be provided with a lysine added to the C-terminus.

The mature ALK3-Fc fusion protein sequence is as follows (SEQ ID NO: 176) and may optionally be provided with a lysine added to the C-terminus.

| (SEQ ID NO: 176) | |
|---|---|
| 1 | GAQNLDSMLH GTGMKSDSDQ KKSENGVTLA PEDTLPFLKC YCSGHCPDDA |
| 51 | INNTCITNGH CFAIIEEDDQ GETTLASGCM KYEGSDFQCK DSPKAQLRRT |
| 101 | IECCRTNLCN QYLQPTLPPV VIGPFFDGSI RTGGGTHTCP PCPAPELLGG |
| 151 | PSVFLFPPKP KDTLMISRTP EVTCVVVDVS HEDPEVKFNW YVDGVEVHNA |
| 201 | KTKPREEQYN STYRVVSVLT VLHQDWLNGK EYKCKVSNKA LPAPIEKTIS |
| 251 | KAKGQPREPQ VYTLPPSRKE MTKNQVSLTC LVKGFYPSDI AVEWESNGQP |
| 301 | ENNYKTTPPV LKSDGSFFLY SKLTVDKSRW QQGNVFSCSV MHEALHNHYT |
| 351 | QKSLSLSPG |

The ALK1-Fc and ALK3-Fc fusion proteins of SEQ ID NO: 126 and SEQ ID NO: 176, respectively, may be co-expressed and purified from a CHO cell line to give rise to a heteromeric complex comprising ALK1-Fc:ALK3-Fc.

### Reverse Electrostatic Approach

In a reverse approach, receptor extracellular domains are linked to the opposite member of the Fc interaction pair compared to the first approach described above. In this example, the alternative polypeptide sequence of the ALK1-Fc fusion protein is provided in Example 36 as SEQ ID NOs: 171-172.

The polypeptide sequence of the ALK3-Fc fusion protein and a nucleic acid sequence encoding it are provided in Example 4 as SEQ ID NOs: 115-117.

The ALK1-Fc and ALK3-Fc fusion proteins of SEQ ID NO: 172 and SEQ ID NO: 117, respectively, may be co-expressed and purified from a CHO cell line to give rise to a variant heteromeric complex comprising ALK1-Fc:ALK3-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ALK1-Fc fusion protein is provided in Example 16 as SEQ ID NOs 413-414.

The complementary form of ALK3-Fc fusion polypeptide (SEQ ID NO: 467) is as follows:

| (SEQ ID NO: 467) | |
|---|---|
| 1 | MDAMKRGLCC VLLLCGAVFV SPGAQNLDSM LHGTGMKSDS DQKKSENGVT |
| 51 | LAPEDTLPFL KCYCSGHCPD DAINNTCITN GHCFAIIEED DQGETTLASG |
| 101 | CMKYEGSDFQ CKDSPKAQLR RTIECCRTNL CNQYLQPTLP PVVIGPFFDG |
| 151 | SIRTGGGTHT CPPCPAPELL GGPSVFLFPP KPKDTLMISR TPEVTCVVVD |
| 201 | VSHEDPEVKF NWYVDGVEVH NAKTKPREEQ YNSTYRVVSV LTVLHQDWLN |
| 251 | GKEYKCKVSN KALPAPIEKT ISKAKGQPRE PQVYTLPPCR EEMTKNQVSL |
| 301 | WCLVKGFYPS DIAVEWESNG QPENNYKTTP PVLDSDGSFF LYSKLTVDKS |
| 351 | RWQQGNVFSC SVMHEALHNH YTQKSLSLSP GK |

The leader and linker sequences are underlined. To guide heterodimer formation with the ALK1-Fc fusion polypeptide of SEQ ID NOs: 413-414, two amino acid substitutions (replacing a serine with a cysteine and a threonine with a tryptophan) can be introduced into the Fc domain of the ALK3-Fc fusion polypeptide as indicated by double underline above. The amino acid sequence of SEQ ID NO: 467 may optionally be provided with the lysine removed from the C-terminus.

The mature ALK3-Fc fusion protein sequence (SEQ ID NO: 468) is as follows and may optionally be provided with the lysine removed from the C-terminus.

| (SEQ ID NO: 468) | |
|---|---|
| 1 | GAQNLDSMLH GTGMKSDSDQ KKSENGVTLA PEDTLPFLKC YCSGHCPDDA |
| 51 | INNTCITNGH CFAIIEEDDQ GETTLASGCM KYEGSDFQCK DSPKAQLRRT |
| 101 | IECCRTNLCN QYLQPTLPPV VIGPFFDGSI RTGGGTHTCP PCPAPELLGG |
| 151 | PSVFLFPPKP KDTLMISRTP EVTCVVVDVS HEDPEVKFNW YVDGVEVHNA |
| 201 | KTKPREEQYN STYRVVSVLT VLHQDWLNGK EYKCKVSNKA LPAPIEKTIS |
| 251 | KAKGQPREPQ VYTLPPCREE MTKNQVSLWC LVKGFYPSDI AVEWESNGQP |
| 301 | ENNYKTTPPV LDSDGSFFLY SKLTVDKSRW QQGNVFSCSV MHEALHNHYT |
| 351 | QKSLSLSPGK |

The ALK1-Fc and ALK3-Fc proteins of SEQ ID NO: 414 and SEQ ID NO: 468, respectively, may be co-expressed and purified from a CHO cell line, to give rise to a variant heteromeric complex comprising ALK1-Fc:ALK3-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, the alternative polypeptide sequence of the ALK1-Fc fusion protein is provided in Example 36 as SEQ ID NOs: 463-464.

The polypeptide sequence of the ALK3-Fc fusion protein is provided in Example 4 as SEQ ID NOs: 407-408.

The ALK1-Fc and ALK3-Fc proteins of SEQ ID NO: 464 and SEQ ID NO: 408, respectively, may be co-expressed and purified from a CHO cell line, to give rise to a variant heteromeric complex comprising ALK1-Fc:ALK3-Fc.

Purification of various ALK1-Fc:ALK3-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 38. Generation of an ALK1-Fc:ALK4-Fc Heterodimer

A soluble ALK1-Fc:ALK4-Fc heteromeric complex can be generated comprising the extracellular domains of human ALK1 and human ALK4, which are each fused to an Fc domain with a linker positioned between the extracellular domain and the Fc domain. The individual constructs are referred to as ALK1-Fc and ALK4-Fc fusion proteins, respectively.

Formation of heteromeric ALK1-Fc:ALK4-Fc may be guided by approaches similar to those described in Example 36.

### Electrostatic Approach

In a first approach, the polypeptide sequence of the ALK1-Fc fusion protein and a nucleic acid sequence encoding it are provided in Example 16 as SEQ ID NOs: 124-126.

The polypeptide sequence of the complementary ALK4-Fc fusion protein (SEQ ID NO: 177) employs the TPA leader and is as follows:

| (SEQ ID NO: 177) | |
|---|---|
| 1 | MDAMKRGLCC VLLLCGAVFV SPGASGPRGV QALLCACTSC LQANYTCETD |
| 51 | GACMVSIFNL DGMEHHVRTC IPKVELVPAG KPFYCLSSED LRNTHCCYTD |
| 101 | YCNRIDLRVP SGHLKEPEHP SMWGPVETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVYTL PPSRKEMTKN QVSLTCLVKG FYPSDIAVEW ESNGQPENNY |
| 301 | KTTPPVLKSD GSFFLYSKLT VDKSRWQQGN VFSCSVMHEA LHNHYTQKSL |
| 351 | SLSPG |

The leader and linker sequences are underlined. To promote formation of the ALK1-Fc:ALK4-Fc heterodimer rather than either of the possible homodimeric complexes, two amino acid substitutions (replacing acidic amino acids with lysines) can be introduced into the Fc domain of the fusion protein as indicated by double underline above. The amino acid sequence of SEQ ID NO: 177 may optionally be provided with a lysine added to the C-terminus.

The mature ALK4-Fc fusion protein sequence is as follows (SEQ ID NO: 178) and may optionally be provided with a lysine added to the C-terminus.

| (SEQ ID NO: 178) | |
|---|---|
| 1 | SGPRGVQALL CACTSCLQAN YTCETDGACM VSIFNLDGME HHVRTCIPKV |
| 51 | ELVPAGKPFY CLSSEDLRNT HCCYTDYCNR IDLRVPSGHL KEPEHPSMWG |
| 101 | PVETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVYTLPPSR KEMTKNQVSL |
| 251 | TCLVKGFYPS DIAVEWESNG QPENNYKTTP PVLKSDGSFF LYSKLTVDKS |
| 301 | RWQQGNVFSC SVMHEALHNH YTQKSLSLSP G |

The ALK1-Fc and ALK4-Fc fusion proteins of SEQ ID NO: 126 and SEQ ID NO: 178, respectively, may be co-expressed and purified from a CHO cell line to give rise to a heteromeric complex comprising ALK1-Fc:ALK4-Fc.

### Reverse Electrostatic Approach

In a reverse approach, receptor extracellular domains are linked to the opposite member of the Fc interaction pair compared to the first approach described above. In this example, the alternative polypeptide sequence of the ALK1-Fc fusion protein is provided in Example 36 as SEQ ID NOs: 171-172.

The polypeptide sequence of the ALK4-Fc fusion protein and a nucleic acid sequence encoding it are provided in Example 1 as SEQ ID NOs: 104-106.

The ALK1-Fc and ALK4-Fc fusion proteins of SEQ ID NO: 172 and SEQ ID NO: 106, respectively, may be co-expressed and purified from a CHO cell line to give rise to a variant heteromeric complex comprising ALK1-Fc:ALK4-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ALK1-Fc fusion protein is provided in Example 16 as SEQ ID NOs 413-414.

The complementary form of ALK4-Fc fusion polypeptide (SEQ ID NO: 469) is as follows:

| (SEQ ID NO: 469) | |
|---|---|
| 1 | MDAMKRGLCC VLLLCGAVFV SPGASGPRGV QALLCACTSC LQANYTCETD |
| 51 | GACMVSIFNL DGMEHHVRTC IPKVELVPAG KPFYCLSSED LRNTHCCYTD |
| 101 | YCNRIDLRVP SGHLKEPEHP SMWGPVETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVYTL PPCREEMTKN QVSLWCLVKG FYPSDIAVEW ESNGQPENNY |
| 301 | KTTPPVLDSD GSFFLYSKLT VDKSRWQQGN VFSCSVMHEA LHNHYTQKSL |
| 351 | SLSPGK |

The leader and linker sequences are underlined. To guide heterodimer formation with the ALK1-Fc fusion polypeptide of SEQ ID NOs: 413-414, two amino acid substitutions (replacing a serine with a cysteine and a threonine with a tryptophan) can be introduced into the Fc domain of the ALK4-Fc fusion polypeptide as indicated by double underline above. The amino acid sequence of SEQ ID NO: 469 may optionally be provided with the lysine removed from the C-terminus.

The mature ALK4-Fc fusion protein sequence (SEQ ID NO: 470) is as follows and may optionally be provided with the lysine removed from the C-terminus.

| (SEQ ID NO: 470) | |
|---|---|
| 1 | SGPRGVQALL CACTSCLQAN YTCETDGACM VSIFNLDGME HHVRTCIPKV |
| 51 | ELVPAGKPFY CLSSEDLRNT HCCYTDYCNR IDLRVPSGHL KEPEHPSMWG |
| 101 | PVETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVYTLPPCR EEMTKNQVSL |
| 251 | WCLVKGFYPS DIAVEWESNG QPENNYKTTP PVLDSDGSFF LYSKLTVDKS |
| 301 | RWQQGNVFSC SVMHEALHNH YTQKSLSLSP GK |

The ALK1-Fc and ALK4-Fc proteins of SEQ ID NO: 414 and SEQ ID NO: 470, respectively, may be co-expressed and purified from a CHO cell line, to give rise to a variant heteromeric complex comprising ALK1-Fc:ALK4-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, the alternative polypeptide sequence of the ALK1-Fc fusion protein is provided in Example 36 as SEQ ID NOs: 463-464.

The polypeptide sequence of the ALK4-Fc fusion protein is provided in Example 1 as SEQ ID NOs: 403-404.

The ALK1-Fc and ALK4-Fc proteins of SEQ ID NO: 464 and SEQ ID NO: 404, respectively, may be co-expressed and purified from a CHO cell line, to give rise to a variant heteromeric complex comprising ALK1-Fc:ALK4-Fc.

Purification of various ALK1-Fc:ALK4-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 39. Generation of an ALK1-Fc:ALK5-Fc Heterodimer

A soluble ALK1-Fc:ALK5-Fc heteromeric complex can be generated comprising the extracellular domains of human ALK1 and human ALK5, which are each fused to an Fc domain with a linker positioned between the extracellular domain and the Fc domain. The individual constructs are referred to as ALK1-Fc and ALK5-Fc fusion proteins, respectively.

Formation of heteromeric ALK1-Fc:ALK5-Fc may be guided by approaches similar to those described in Example 36.

### Electrostatic Approach

In a first approach, the polypeptide sequence of the ALK1-Fc fusion protein and a nucleic acid sequence encoding it are provided in Example 16 as SEQ ID NOs: 124-126.

The polypeptide sequence of the complementary ALK5-Fc fusion protein (SEQ ID NO: 179) employs the TPA leader and is as follows:

| (SEQ ID NO: 179) | |
|---|---|
| 1 | MDAMKRGLCC VLLLCGAVFV SPGAALLPGA TALQCFCHLC TKDNFTCVTD |
| 51 | GLCFVSVTET TDKVIHNSMC IAEIDLIPRD RPFVCAPSSK TGSVTTTYCC |
| 101 | NQDHCNKIEL PTTVKSSPGL GPVETGGGTH TCPPCPAPEL LGGPSVFLFP |
| 151 | PKPKDTLMIS RTPEVTCVVV DVSHEDPEVK FNWYVDGVEV HNAKTKPREE |
| 201 | QYNSTYRVVS VLTVLHQDWL NGKEYKCKVS NKALPAPIEK TISKAKGQPR |
| 251 | EPQVYTLPPS RKEMTKNQVS LTCLVKGFYP SDIAVEWESN GQPENNYKTT |
| 301 | PPVLKSDGSF FLYSKLTVDK SRWQQGNVFS CSVMHEALHN HYTQKSLSLS |
| 351 | PG |

The leader and linker sequences are underlined. To promote formation of the ALK1-Fc:ALK5-Fc heterodimer rather than either of the possible homodimeric complexes, two amino acid substitutions (replacing acidic amino acids with lysines) can be introduced into the Fc domain of the fusion protein as indicated by double underline above. The amino acid sequence of SEQ ID NO: 179 may optionally be provided with a lysine added to the C-terminus.

The mature ALK5-Fc fusion protein sequence is as follows (SEQ ID NO: 180) and may optionally be provided with a lysine added to the C-terminus.

| (SEQ ID NO: 180) | |
|---|---|
| 1 | ALLPGATALQ CFCHLCTKDN FTCVTDGLCF VSVTETTDKV IHNSMCIAEI |
| 51 | DLIPRDRPFV CAPSSKTGSV TTTYCCNQDH CNKIELPTTV KSSPGLGPVE |
| 101 | TGGGTHTCPP CPAPELLGGP SVFLFPPKPK DTLMISRTPE VTCVVVDVSH |
| 151 | EDPEVKFNWY VDGVEVHNAK TKPREEQYNS TYRVVSVLTV LHQDWLNGKE |
| 201 | YKCKVSNKAL PAPIEKTISK AKGQPREPQV YTLPPSRKEM TKNQVSLTCL |
| 251 | VKGFYPSDIA VEWESNGQPE NNYKTTPPVL KSDGSFFLYS KLTVDKSRWQ |
| 301 | QGNVFSCSVM HEALHNHYTQ KSLSLSPG |

The ALK1-Fc and ALK5-Fc fusion proteins of SEQ ID NO: 126 and SEQ ID NO: 180, respectively, may be co-expressed and purified from a CHO cell line to give rise to a heteromeric complex comprising ALK1-Fc:ALK5-Fc.

### Reverse Electrostatic Approach

In a reverse approach, receptor extracellular domains are linked to the opposite member of the Fc interaction pair compared to the first approach described above. In this example, the alternative polypeptide sequence of the ALK1-Fc fusion protein is provided in Example 36 as SEQ ID NOs: 171-172.

The polypeptide sequence of the ALK5-Fc fusion protein and a nucleic acid sequence encoding it are provided in Example 11 as SEQ ID NOs: 139-141.

The ALK1-Fc and ALK5-Fc fusion proteins of SEQ ID NO: 172 and SEQ ID NO: 141, respectively, may be co-expressed and purified from a CHO cell line to give rise to a variant heteromeric complex comprising ALK1-Fc:ALK5-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ALK1-Fc fusion protein is provided in Example 16 as SEQ ID NOs 413-414.

The complementary form of ALK5-Fc fusion polypeptide (SEQ ID NO: 471) is as follows:

| (SEQ ID NO: 471) | |
|---|---|
| 1 | MDAMKRGLCC VLLLCGAVFV SPGAALLPGA TALQCFCHLC TKDNFTCVTD |
| 51 | GLCFVSVTET TDKVIHNSMC IAEIDLIPRD RPFVCAPSSK TGSVTTTYCC |
| 101 | NQDHCNKIEL PTTVKSSPGL GPVETGGGTH TCPPCPAPEL LGGPSVFLFP |
| 151 | PKPKDTLMIS RTPEVTCVVV DVSHEDPEVK FNWYVDGVEV HNAKTKPREE |
| 201 | QYNSTYRVVS VLTVLHQDWL NGKEYKCKVS NKALPAPIEK TISKAKGQPR |
| 251 | EPQVYTLPPC REEMTKNQVS LWCLVKGFYP SDIAVEWESN GQPENNYKTT |
| 301 | PPVLDSDGSF FLYSKLTVDK SRWQQGNVFS CSVMHEALHN HYTQKSLSLS |
| 351 | PGK |

The leader and linker sequences are underlined. To guide heterodimer formation with the ALK1-Fc fusion polypeptide of SEQ ID NOs: 413-414, two amino acid substitutions (replacing a serine with a cysteine and a threonine with a tryptophan) can be introduced into the Fc domain of the ALK5-Fc fusion polypeptide as indicated by double underline above. The amino acid sequence of SEQ ID NO: 471 may optionally be provided with the lysine removed from the C-terminus.

The mature ALK5-Fc fusion protein sequence (SEQ ID NO: 472) is as follows and may optionally be provided with the lysine removed from the C-terminus.

| (SEQ ID NO: 472) | |
|---|---|
| 1 | ALLPGATALQ CFCHLCTKDN FTCVTDGLCF VSVTETTDKV IHNSMCIAEI |
| 51 | DLIPRDRPFV CAPSSKTGSV TTTYCCNQDH CNKIELPTTV KSSPGLGPVE |
| 101 | TGGGTHTCPP CPAPELLGGP SVFLFPPKPK DTLMISRTPE VTCVVVDVSH |
| 151 | EDPEVKFNWY VDGVEVHNAK TKPREEQYNS TYRVVSVLTV LHQDWLNGKE |
| 201 | YKCKVSNKAL PAPIEKTISK AKGQPREPQV YTLPPCREEM TKNQVSLWCL |
| 251 | VKGFYPSDIA VEWESNGQPE NNYKTTPPVL DSDGSFFLYS KLTVDKSRWQ |
| 301 | QGNVFSCSVM HEALHNHYTQ KSLSLSPGK |

The ALK1-Fc and ALK5-Fc proteins of SEQ ID NO: 414 and SEQ ID NO: 472, respectively, may be co-expressed and purified from a CHO cell line, to give rise to a variant heteromeric complex comprising ALK1-Fc:ALK5-Fc.

### Reverse Hydrophobic Approach

In another approach to promoting the formation of heteromultimer complexes using asymmetric Fc fusion proteins, the Fe domains are altered to introduce complementary hydrophobic interactions and an additional intermolecular disulfide bond as described immediately above. However, receptor extracellular domains in this approach are linked to the opposite member of the Fc interaction pair compared to the above approach. In this example, the alternative polypeptide sequence of the ALK1-Fc fusion protein is provided in Example 36 as SEQ ID NOs: 463-464.

The polypeptide sequence of the ALK5-Fc fusion protein is provided in Example 16 as SEQ ID NOs: 423-424.

The ALK1-Fc and ALK5-Fc proteins of SEQ ID NO: 464 and SEQ ID NO: 424, respectively, may be co-expressed and purified from a CHO cell line, to give rise to a variant heteromeric complex comprising ALK1-Fc:ALK5-Fc.

Purification of various ALK1-Fc:ALK5-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 40. Generation of an ALK1-Fc:ALK6-Fc Heterodimer

A soluble ALK1-Fc:ALK6-Fc heteromeric complex can be generated comprising the extracellular domains of human ALK1 and human ALK6, which are each fused to an Fc domain with a linker positioned between the extracellular domain and the Fc domain. The individual constructs are referred to as ALK1-Fc and ALK6-Fc fusion proteins, respectively.

Formation of heteromeric ALK1-Fc:ALK6-Fc may be guided by approaches similar to those described in Example 36.

### Electrostatic Approach

In a first approach, the polypeptide sequence of the ALK1-Fc fusion protein and a nucleic acid sequence encoding it are provided in Example 16 as SEQ ID NOs: 124-126.

The polypeptide sequence of the complementary ALK6-Fc fusion protein (SEQ ID NO: 181) employs the TPA leader and is as follows:

| (SEQ ID NO: 181) | |
|---|---|
| 1 | MDAMKRGLCC VLLLCGAVFV SPGAKKEDGE STAPTPRPKV LRCKCHHHCP |
| 51 | EDSVNNICST DGYCFTMIEE DDSGLPVVTS GCLGLEGSDF QCRDTPIPHQ |
| 101 | RRSIECCTER NECNKDLHPT LPPLKNRDFV DGPIHHRTGG GTHTCPPCPA |
| 151 | PELLGGPSVF LFPPKPKDTL MISRTPEVTC VVVDVSHEDP EVKFNWYVDG |
| 201 | VEVHNAKTKP REEQYNSTYR VVSVLTVLHQ DWLNGKEYKC KVSNKALPAP |
| 251 | IEKTISKAKG QPREPQVYTL PPSRKEMTKN QVSLTCLVKG FYPSDIAVEW |
| 301 | ESNGQPENNY KTTPPVLKSD GSFFLYSKLT VDKSRWQQGN VFSCSVMHEA |
| 351 | LHNHYTQKSL SLSPG |

The leader and linker sequences are underlined. To promote formation of the ALK1-Fc:ALK6-Fc heterodimer rather than either of the possible homodimeric complexes, two amino acid substitutions (replacing acidic amino acids with lysines) can be introduced into the Fc domain of the fusion protein as indicated by double underline above. The amino acid sequence of SEQ ID NO: 181 may optionally be provided with a lysine added to the C-terminus.

The mature ALK6-Fc fusion protein sequence is as follows (SEQ ID NO: 182) and may optionally be provided with a lysine added to the C-terminus.

| (SEQ ID NO: 182) | |
|---|---|
| 1 | KKEDGESTAP TPRPKVLRCK CHHHCPEDSV NNICSTDGYC FTMIEEDDSG |
| 51 | LPVVTSGCLG LEGSDFQCRD TPIPHQRRSI ECCTERNECN KDLHPTLPPL |
| 101 | KNRDFVDGPI HHRTGGGTHT CPPCPAPELL GGPSVFLFPP KPKDTLMISR |
| 151 | TPEVTCVVVD VSHEDPEVKF NWYVDGVEVH NAKTKPREEQ YNSTYRVVSV |
| 201 | LTVLHQDWLN GKEYKCKVSN KALPAPIEKT ISKAKGQPRE PQVYTLPPSR |
| 251 | KEMTKNQVSL TCLVKGFYPS DIAVEWESNG QPENNYKTTP PVLKSDGSFF |
| 301 | LYSKLTVDKS RWQQGNVFSC SVMHEALHNH YTQKSLSLSP G |

The ALK1-Fc and ALK6-Fc fusion proteins of SEQ ID NO: 126 and SEQ ID NO: 182, respectively, may be co-expressed and purified from a CHO cell line to give rise to a heteromeric complex comprising ALK1-Fc:ALK6-Fc.

### Reverse Electrostatic Approach

In a reverse approach, receptor extracellular domains are linked to the opposite member of the Fc interaction pair compared to the first approach described above. In this example, the alternative polypeptide sequence of the ALK1-Fc fusion protein is provided in Example 36 as SEQ ID NOs: 171-172.

The polypeptide sequence of the ALK6-Fc fusion protein and a nucleic acid sequence encoding it are provided in Example 13 as SEQ ID NOs: 142-144.

The ALK1-Fc and ALK6-Fc fusion proteins of SEQ ID NO: 172 and SEQ ID NO: 144, respectively, may be co-expressed and purified from a CHO cell line to give rise to a variant heteromeric complex comprising ALK1-Fc:ALK6-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ALK1-Fc fusion protein is provided in Example 16 as SEQ ID NOs 413-414.

The complementary form of ALK6-Fc fusion polypeptide (SEQ ID NO: 473) is as follows:

| (SEQ ID NO: 473) | |
|---|---|
| 1 | MDAMKRGLCC VLLLCGAVFV SPGAKKEDGE STAPTPRPKV LRCKCHHHCP |
| 51 | EDSVNNICST DGYCFTMIEE DDSGLPVVTS GCLGLEGSDF QCRDTPIPHQ |
| 101 | RRSIECCTER NECNKDLHPT LPPLKNRDFV DGPIHHRTGG GTHTCPPCPA |
| 151 | PELLGGPSVF LFPPKPKDTL MISRTPEVTC VVVDVSHEDP EVKFNWYVDG |
| 201 | VEVHNAKTKP REEQYNSTYR VVSVLTVLHQ DWLNGKEYKC KVSNKALPAP |
| 251 | IEKTISKAKG QPREPQVYTL PPCREEMTKN QVSLWCLVKG FYPSDIAVEW |
| 301 | ESNGQPENNY KTTPPVLDSD GSFFLYSKLT VDKSRWQQGN VFSCSVMHEA |
| 351 | LHNHYTQKSL SLSPGK |

The leader and linker sequences are underlined. To guide heterodimer formation with the ALK1-Fc fusion polypeptide of SEQ ID NOs: 413-414, two amino acid substitutions (replacing a serine with a cysteine and a threonine with a tryptophan) can be introduced into the Fc domain of the ALK6-Fc fusion polypeptide as indicated by double underline above. The amino acid sequence of SEQ ID NO: 473 may optionally be provided with the lysine removed from the C-terminus.

The mature ALK6-Fc fusion protein sequence (SEQ ID NO: 474) is as follows and may optionally be provided with the lysine removed from the C-terminus.

| (SEQ ID NO: 474) | |
|---|---|
| 1 | KKEDGESTAP TPRPKVLRCK CHHHCPEDSV NNICSTDGYC FTMIEEDDSG |
| 51 | LPVVTSGCLG LEGSDFQCRD TPIPHQRRSI ECCTERNECN KDLHPTLPPL |
| 101 | KNRDFVDGPI HHRTGGGTHT CPPCPAPELL GGPSVFLFPP KPKDTLMISR |
| 151 | TPEVTCVVVD VSHEDPEVKF NWYVDGVEVH NAKTKPREEQ YNSTYRVVSV |
| 201 | LTVLHQDWLN GKEYKCKVSN KALPAPIEKT ISKAKGQPRE PQVYTLPPCR |
| 251 | EEMTKNQVSL WCLVKGFYPS DIAVEWESNG QPENNYKTTP PVLDSDGSFF |
| 301 | LYSKLTVDKS RWQQGNVFSC SVMHEALHNH YTQKSLSLSP GK |

The ALK1-Fc and ALK6-Fc proteins of SEQ ID NO: 414 and SEQ ID NO: 474, respectively, may be co-expressed and purified from a CHO cell line, to give rise to a variant heteromeric complex comprising ALK1-Fc:ALK6-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, the alternative polypeptide sequence of the ALK1-Fc fusion protein is provided in Example 36 as SEQ ID NOs: 463-464.

The polypeptide sequence of the ALK6-Fc fusion protein is provided in Example 13 as SEQ ID NOs: 425-426.

The ALK1-Fc and ALK6-Fc proteins of SEQ ID NO: 464 and SEQ ID NO: 426, respectively, may be co-expressed and purified from a CHO cell line, to give rise to a variant heteromeric complex comprising ALK1-Fc:ALK6-Fc.

Purification of various ALK1-Fc:ALK6-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 41. Generation of an ALK1-Fc:ALK7-Fc Heterodimer

A soluble ALK1-Fc:ALK7-Fc heteromeric complex can be generated comprising the extracellular domains of human ALK1 and human ALK7, which are each fused to an Fc domain with a linker positioned between the extracellular domain and the Fc domain. The individual constructs are referred to as ALK1-Fc and ALK7-Fc fusion proteins, respectively.

Formation of heteromeric ALK1-Fc:ALK7-Fc may be guided by approaches similar to those described in Example 36.

### Electrostatic Approach

In a first approach, the polypeptide sequence of the ALK1-Fc fusion protein and a nucleic acid sequence encoding it are provided in Example 16 as SEQ ID NOs: 124-126.

The polypeptide sequence of the complementary ALK7-Fc fusion protein (SEQ ID NO: 183) employs the TPA leader and is as follows:

| (SEQ ID NO: 183) | |
|---|---|
| 1 | MDAMKRGLCC VLLLCGAVFV SPGAGLKCVC LLCDSSNFTC QTEGACWASV |
| 51 | MLTNGKEQVI KSCVSLPELN AQVFCHSSNN VTKTECCFTD FCNNITLHLP |
| 101 | TASPNAPKLG PMETGGGTHT CPPCPAPELL GGPSVFLFPP KPKDTLMISR |
| 151 | TPEVTCVVVD VSHEDPEVKF NWYVDGVEVH NAKTKPREEQ YNSTYRVVSV |
| 201 | LTVLHQDWLN GKEYKCKVSN KALPAPIEKT ISKAKGQPRE PQVYTLPPSR |
| 251 | KEMTKNQVSL TCLVKGFYPS DIAVEWESNG QPENNYKTTP PVLKSDGSFF |
| 301 | LYSKLTVDKS RWQQGNVFSC SVMHEALHNH YTQKSLSLSP G |

The leader and linker sequences are underlined. To promote formation of the ALK1-Fc:ALK7-Fc heterodimer rather than either of the possible homodimeric complexes, two amino acid substitutions (replacing acidic amino acids with lysines) can be introduced into the Fc domain of the fusion protein as indicated by double underline above. The amino acid sequence of SEQ ID NO: 183 may optionally be provided with a lysine added to the C-terminus.

The mature ALK7-Fc fusion protein sequence is expected to be as follows (SEQ ID NO: 184) and may optionally be provided with a lysine added to the C-terminus.

| (SEQ ID NO: 184) | |
|---|---|
| 1 | GLKCVCLLCD SSNFTCQTEG ACWASVMLTN GKEQVIKSCV SLPELNAQVF |
| 51 | CHSSNNVTKT ECCFTDFCNN ITLHLPTASP NAPKLGPMET GGGTHTCPPC |
| 101 | PAPELLGGPS VFLFPPKPKD TLMISRTPEV TCVVVDVSHE DPEVKFNWYV |
| 151 | DGVEVHNAKT KPREEQYNST YRVVSVLTVL HQDWLNGKEY KCKVSNKALP |
| 201 | APIEKTISKA KGQPREPQVY TLPPSRKEMT KNQVSLTCLV KGFYPSDIAV |
| 251 | EWESNGQPEN NYKTTPPVLK SDGSFFLYSK LTVDKSRWQQ GNVFSCSVMH |
| 301 | EALHNHYTQK SLSLSPG |

The ALK1-Fc and ALK7-Fc fusion proteins of SEQ ID NO: 126 and SEQ ID NO: 184, respectively, may be co-expressed and purified from a CHO cell line to give rise to a heteromeric complex comprising ALK1-Fc:ALK7-Fc.

### Reverse Electrostatic Approach

In a reverse approach, receptor extracellular domains are linked to the opposite member of the Fc interaction pair compared to the first approach described above. In this example, the alternative polypeptide sequence of the ALK1-Fc fusion protein is provided in Example 36 as SEQ ID NOs: 171-172.

The polypeptide sequence of the ALK7-Fc fusion protein and a nucleic acid sequence encoding it are provided in Example 7 as SEQ ID NOs: 112-114.

The ALK1-Fc and ALK7-Fc fusion proteins of SEQ ID NO: 172 and SEQ ID NO: 114, respectively, may be co-expressed and purified from a CHO cell line to give rise to a variant heteromeric complex comprising ALK1-Fc:ALK7-Fc.

### Hydrophobic Approach

In another approach to promoting the formation of heteromultimer complexes using asymmetric Fc fusion proteins, the Fc domains are altered to introduce complementary hydrophobic interactions and an additional intermolecular disulfide bond. The polypeptide sequence of the ALK1-Fc fusion protein is provided in Example 16 as SEQ ID NOs 413-414.

The complementary form of ALK7-Fc fusion polypeptide (SEQ ID NO: 475) is as follows:

| (SEQ ID NO: 475) | |
|---|---|
| 1 | MDAMKRGLCC VLLLCGAVFV SPGAGLKCVC LLCDSSNFTC QTEGACWASV |
| 51 | MLTNGKEQVI KSCVSLPELN AQVFCHSSNN VTKTECCFTD FCNNITLHLP |
| 101 | TASPNAPKLG PMETGGGTHT CPPCPAPELL GGPSVFLFPP KPKDTLMISR |
| 151 | TPEVTCVVVD VSHEDPEVKF NWYVDGVEVH NAKTKPREEQ YNSTYRVVSV |
| 201 | LTVLHQDWLN GKEYKCKVSN KALPAPIEKT ISKAKGQPRE PQVYTLPPCR |
| 251 | EEMTKNQVSL WCLVKGFYPS DIAVEWESNG QPENNYKTTP PVLDSDGSFF |
| 301 | LYSKLTVDKS RWQQGNVFSC SVMHEALHNH YTQKSLSLSP GK |

The leader and linker sequences are underlined. To guide heterodimer formation with the ALK1-Fc fusion polypeptide of SEQ ID NOs: 413-414, two amino acid substitutions (replacing a serine with a cysteine and a threonine with a tryptophan) can be introduced into the Fc domain of the ALK7-Fc fusion polypeptide as indicated by double underline above. The amino acid sequence of SEQ ID NO: 475 may optionally be provided with the lysine removed from the C-terminus.

The mature ALK7-Fc fusion protein sequence (SEQ ID NO: 476) is expected to be as follows and may optionally be provided with the lysine removed from the C-terminus.

| (SEQ ID NO: 476) | |
|---|---|
| 1 | GLKCVCLLCD SSNFTCQTEG ACWASVMLTN GKEQVIKSCV SLPELNAQVF |
| 51 | CHSSNNVTKT ECCFTDFCNN ITLHLPTASP NAPKLGPMET GGGTHTCPPC |
| 101 | PAPELLGGPS VFLFPPKPKD TLMISRTPEV TCVVVDVSHE DPEVKFNWYV |
| 151 | DGVEVHNAKT KPREEQYNST YRVVSVLTVL HQDWLNGKEY KCKVSNKALP |
| 201 | APIEKTISKA KGQPREPQVY TLPPCREEMT KNQVSLWCLV KGFYPSDIAV |
| 251 | EWESNGQPEN NYKTTPPVLD SDGSFFLYSK LTVDKSRWQQ GNVFSCSVMH |
| 301 | EALHNHYTQK SLSLSPGK |

The ALK1-Fc and ALK7-Fc proteins of SEQ ID NO: 414 and SEQ ID NO: 476, respectively, may be co-expressed and purified from a CHO cell line, to give rise to a variant heteromeric complex comprising ALK1-Fc:ALK7-Fc.

### Reverse Hydrophobic Approach

In another approach to promoting the formation of heteromultimer complexes using asymmetric Fc fusion proteins, the Fc domains are altered to introduce complementary hydrophobic interactions and an additional intermolecular disulfide bond as described immediately above. However, receptor extracellular domains in this approach are linked to the opposite member of the Fc interaction pair compared to the above approach. In this example, the alternative polypeptide sequence of the ALK1-Fc fusion protein is provided in Example 36 as SEQ ID NOs: 463-464.

The polypeptide sequence of the ALK7-Fc fusion protein is provided in Example 7 as SEQ ID NOs: 405-406.

The ALK1-Fc and ALK7-Fc proteins of SEQ ID NO: 464 and SEQ ID NO: 406, respectively, may be co-expressed and purified from a CHO cell line, to give rise to a variant heteromeric complex comprising ALK1-Fc:ALK7-Fc.

Purification of various ALK1-Fc:ALK7-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 42. Generation of Additional Heterodimers Comprising Two Type I Receptors

Formation of additional heterdimeric protein complexes comprising extracellular domains of two type I receptors may be guided by approaches similar to those described in Example 36. Amino acid SEQ ID NOs for these variant heterodimers are disclosed in the following table, which for completeness also includes the heterodimers already discussed that comprise two type I receptors (Examples 36-41). As disclosed in Examples 36-41, the C-terminal lysine of each receptor-Fc fusion protein may optionally be included or omitted.

In each case, a soluble heteromeric complex can be generated comprising the extracellular domains of two different type I receptors, which are each fused to an Fc domain with a linker positioned between the extracellular domain and the Fc domain. In each case, the fusion proteins may be co-expressed and purified from a CHO cell line to give rise to a variant heteromeric protein complex. In each case, purification of various heteromeric protein complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

TABLE
“Type I:Type I Receptor Heterodimers”
Amino Acid SEQ ID NOs for Type I:Type I Receptor Heterodimers

| | | | | Amino Acid | |
|---|---|---|---|---|---|
| Heteromeric | | ECD | | SEQ ID NO | |
| Fusion Protein | | Correspondence | Receptor-Fc | With | |
| Complex | Fc Pair Type | to Fc Pair | Fusion Protein | Leader | Mature |
| ALK1-Fc:ALK2-Fc | Electrostatic | A | ALK1-Fc | 124 | 126 |
| | | | ALK2-Fc | 173 | 174 |
| | | B | ALK1-Fc | 171 | 172 |
| | | | ALK2-Fc | 136 | 138 |
| | Hydrophobic | A | ALK1-Fc | 413 | 414 |
| | | | ALK2-Fc | 465 | 466 |
| | | B | ALK1-Fc | 463 | 464 |
| | | | ALK2-Fc | 421 | 422 |
| ALK1-Fc:ALK3-Fc | Electrostatic | A | ALK1-Fc | 124 | 126 |
| | | | ALK3-Fc | 175 | 176 |
| | | B | ALK1-Fc | 171 | 172 |
| | | | ALK3-Fc | 115 | 117 |
| | Hydrophobic | A | ALK1-Fc | 413 | 414 |
| | | | ALK3-Fc | 467 | 468 |
| | | B | ALK1-Fc | 463 | 464 |
| | | | ALK3-Fc | 407 | 408 |
| ALK1-Fc:ALK4-Fc | Electrostatic | A | ALK1-Fc | 124 | 126 |
| | | | ALK4-Fc | 177 | 178 |
| | | B | ALK1-Fc | 171 | 172 |
| | | | ALK4-Fc | 104 | 106 |
| | Hydrophobic | A | ALK1-Fc | 413 | 414 |
| | | | ALK4-Fc | 469 | 470 |
| | | B | ALK1-Fc | 463 | 464 |
| | | | ALK4-Fc | 403 | 404 |
| ALK1-Fc:ALK5-Fc | Electrostatic | A | ALK1-Fc | 124 | 126 |
| | | | ALK5-Fc | 179 | 180 |
| | | B | ALK1-Fc | 171 | 172 |
| | | | ALK5-Fc | 139 | 141 |
| | Hydrophobic | A | ALK1-Fc | 413 | 414 |
| | | | ALK5-Fc | 471 | 472 |
| | | B | ALK1-Fc | 463 | 464 |
| | | | ALK5-Fc | 423 | 424 |
| ALK1-Fc:ALK6-Fc | Electrostatic | A | ALK1-Fc | 124 | 126 |
| | | | ALK6-Fc | 181 | 182 |
| | | B | ALK1-Fc | 171 | 172 |
| | | | ALK6-Fc | 142 | 144 |
| | Hydrophobic | A | ALK1-Fc | 413 | 414 |
| | | | ALK6-Fc | 473 | 474 |
| | | B | ALK1-Fc | 463 | 464 |
| | | | ALK6-Fc | 425 | 426 |
| ALK1-Fc:ALK7-Fc | Electrostatic | A | ALK1-Fc | 124 | 126 |
| | | | ALK7-Fc | 183 | 184 |
| | | B | ALK1-Fc | 171 | 172 |
| | | | ALK7-Fc | 112 | 114 |
| | Hydrophobic | A | ALK1-Fc | 413 | 414 |
| | | | ALK7-Fc | 475 | 476 |
| | | B | ALK1-Fc | 463 | 464 |
| | | | ALK7-Fc | 405 | 406 |
| ALK2-Fc:ALK3-Fc | Electrostatic | A | ALK2-Fc | 136 | 138 |
| | | | ALK3-Fc | 175 | 176 |
| | | B | ALK2-Fc | 173 | 174 |
| | | | ALK3-Fc | 115 | 117 |
| | Hydrophobic | A | ALK2-Fc | 421 | 422 |
| | | | ALK3-Fc | 467 | 468 |
| | | B | ALK2-Fc | 465 | 466 |
| | | | ALK3-Fc | 407 | 408 |
| ALK2-Fc:ALK4-Fc | Electrostatic | A | ALK2-Fc | 136 | 138 |
| | | | ALK4-Fc | 177 | 178 |
| | | B | ALK2-Fc | 173 | 174 |
| | | | ALK4-Fc | 104 | 106 |
| | Hydrophobic | A | ALK2-Fc | 421 | 422 |
| | | | ALK4-Fc | 469 | 470 |
| | | B | ALK2-Fc | 465 | 466 |
| | | | ALK4-Fc | 403 | 404 |
| ALK2-Fc:ALK5-Fc | Electrostatic | A | ALK2-Fc | 136 | 138 |
| | | | ALK5-Fc | 179 | 180 |
| | | B | ALK2-Fc | 173 | 174 |
| | | | ALK5-Fc | 139 | 141 |
| | Hydrophobic | A | ALK2-Fc | 421 | 422 |
| | | | ALK5-Fc | 471 | 472 |
| | | B | ALK2-Fc | 465 | 466 |
| | | | ALK5-Fc | 423 | 424 |
| ALK2-Fc:ALK6-Fc | Electrostatic | A | ALK2-Fc | 136 | 138 |
| | | | ALK6-Fc | 181 | 182 |
| | | B | ALK2-Fc | 173 | 174 |
| | | | ALK6-Fc | 142 | 144 |
| | Hydrophobic | A | ALK2-Fc | 421 | 422 |
| | | | ALK6-Fc | 473 | 474 |
| | | B | ALK2-Fc | 465 | 466 |
| | | | ALK6-Fc | 425 | 426 |
| ALK2-Fc:ALK7-Fc | Electrostatic | A | ALK2-Fc | 136 | 138 |
| | | | ALK7-Fc | 183 | 184 |
| | | B | ALK2-Fc | 173 | 174 |
| | | | ALK7-Fc | 112 | 114 |
| | Hydrophobic | A | ALK2-Fc | 421 | 422 |
| | | | ALK7-Fc | 475 | 476 |
| | | B | ALK2-Fc | 465 | 466 |
| | | | ALK7-Fc | 405 | 406 |
| ALK3-Fc:ALK4-Fc | Electrostatic | A | ALK3-Fc | 115 | 117 |
| | | | ALK4-Fc | 177 | 178 |
| | | B | ALK3-Fc | 175 | 176 |
| | | | ALK4-Fc | 104 | 106 |
| | Hydrophobic | A | ALK3-Fc | 407 | 408 |
| | | | ALK4-Fc | 469 | 470 |
| | | B | ALK3-Fc | 467 | 468 |
| | | | ALK4-Fc | 403 | 404 |
| ALK3-Fc:ALK5-Fc | Electrostatic | A | ALK3-Fc | 115 | 117 |
| | | | ALK5-Fc | 179 | 180 |
| | | B | ALK3-Fc | 175 | 176 |
| | | | ALK5-Fc | 139 | 141 |
| | Hydrophobic | A | ALK3-Fc | 407 | 408 |
| | | | ALK5-Fc | 471 | 472 |
| | | B | ALK3-Fc | 467 | 468 |
| | | | ALK5-Fc | 423 | 424 |
| ALK3-Fc:ALK6-Fc | Electrostatic | A | ALK3-Fc | 115 | 117 |
| | | | ALK6-Fc | 181 | 182 |
| | | B | ALK3-Fc | 175 | 176 |
| | | | ALK6-Fc | 142 | 144 |
| | Hydrophobic | A | ALK3-Fc | 407 | 408 |
| | | | ALK6-Fc | 473 | 474 |
| | | B | ALK3-Fc | 467 | 468 |
| | | | ALK6-Fc | 425 | 426 |
| ALK3-Fc:ALK7-Fc | Electrostatic | A | ALK3-Fc | 115 | 117 |
| | | | ALK7-Fc | 183 | 184 |
| | | B | ALK3-Fc | 175 | 176 |
| | | | ALK7-Fc | 112 | 114 |
| | Hydrophobic | A | ALK3-Fc | 407 | 408 |
| | | | ALK7-Fc | 475 | 476 |
| | | B | ALK3-Fc | 467 | 468 |
| | | | ALK7-Fc | 405 | 406 |
| ALK4-Fc:ALK5-Fc | Electrostatic | A | ALK4-Fc | 104 | 106 |
| | | | ALK5-Fc | 179 | 180 |
| | | B | ALK4-Fc | 177 | 178 |
| | | | ALK5-Fc | 139 | 141 |
| | Hydrophobic | A | ALK4-Fc | 403 | 404 |
| | | | ALK5-Fc | 471 | 472 |
| | | B | ALK4-Fc | 469 | 470 |
| | | | ALK5-Fc | 423 | 424 |
| ALK4-Fc:ALK6-Fc | Electrostatic | A | ALK4-Fc | 104 | 106 |
| | | | ALK6-Fc | 181 | 182 |
| | | B | ALK4-Fc | 177 | 178 |
| | | | ALK6-Fc | 142 | 144 |
| | Hydrophobic | A | ALK4-Fc | 403 | 404 |
| | | | ALK6-Fc | 473 | 474 |
| | | B | ALK4-Fc | 469 | 470 |
| | | | ALK6-Fc | 425 | 426 |
| ALK4-Fc:ALK7-Fc | Electrostatic | A | ALK4-Fc | 104 | 106 |
| | | | ALK7-Fc | 183 | 184 |
| | | B | ALK4-Fc | 177 | 178 |
| | | | ALK7-Fc | 112 | 114 |
| | Hydrophobic | A | ALK4-Fc | 403 | 404 |
| | | | ALK7-Fc | 475 | 476 |
| | | B | ALK4-Fc | 469 | 470 |
| | | | ALK7-Fc | 405 | 406 |
| ALK5-Fc:ALK6-Fc | Electrostatic | A | ALK5-Fc | 139 | 141 |
| | | | ALK6-Fc | 181 | 182 |
| | | B | ALK5-Fc | 179 | 180 |
| | | | ALK6-Fc | 142 | 144 |
| | Hydrophobic | A | ALK5-Fc | 423 | 424 |
| | | | ALK6-Fc | 473 | 474 |
| | | B | ALK5-Fc | 471 | 472 |
| | | | ALK6-Fc | 425 | 426 |
| ALK5-Fc:ALK7-Fc | Electrostatic | A | ALK5-Fc | 139 | 141 |
| | | | ALK7-Fc | 183 | 184 |
| | | B | ALK5-Fc | 179 | 180 |
| | | | ALK7-Fc | 112 | 114 |
| | Hydrophobic | A | ALK5-Fc | 423 | 424 |
| | | | ALK7-Fc | 475 | 476 |
| | | B | ALK5-Fc | 471 | 472 |
| | | | ALK7-Fc | 405 | 406 |
| ALK6-Fc:ALK7-Fc | Electrostatic | A | ALK6-Fc | 142 | 144 |
| | | | ALK7-Fc | 183 | 184 |
| | | B | ALK6-Fc | 181 | 182 |
| | | | ALK7-Fc | 112 | 114 |
| | Hydrophobic | A | ALK6-Fc | 425 | 426 |
| | | | ALK7-Fc | 475 | 476 |
| | | B | ALK6-Fc | 473 | 474 |
| | | | ALK7-Fc | 405 | 406 |

### Example 43. Generation of Additional Heterodimers Comprising a Type I Receptor and a Type II Receptor

Formation of additional heterdimeric protein complexes comprising extracellular domains of a type I receptor and a type II receptor may be guided by approaches similar to those described in Example 1. Amino acid SEQ ID NOs for these variant heterodimers are disclosed in the following table, which for completeness also includes the heterodimers already discussed that comprise a type I receptor and a type II receptor (Examples 1-29). As disclosed in Examples 1-29, the C-terminal lysine of each receptor-Fc fusion protein may optionally be included or omitted.

In each case, a soluble heteromeric complex can be generated comprising the extracellular domains of a type I receptor and a type II receptor, which are each fused to an Fc domain with a linker positioned between the extracellular domain and the Fc domain. In each case, the fusion proteins may be co-expressed and purified from a CHO cell line to give rise to a variant heteromeric protein complex. In each case, purification of various heteromeric protein complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

TABLE
“Type I:Type II Receptor Heterodimers”
Amino Acid SEQ ID NOs for Type I:Type II Receptor Heterodimers

| | | | | Amino Acid | |
|---|---|---|---|---|---|
| Heteromeric | | ECD | | SEQ ID NO | |
| Fusion Protein | | Correspondence | Receptor-Fc | With | |
| Complex | Fc Pair Type | to Fc Pair | Fusion Protein | Leader | Mature |
| ActRIIA-Fc:ALK1-Fc | Electrostatic | A | ActRIIA-Fc | 118 | 120 |
| | | | ALK1-Fc | 124 | 126 |
| | | B | ActRIIA-Fc | 151 | 152 |
| | | | ALK1-Fc | 171 | 172 |
| | Hydrophobic | A | ActRIIA-Fc | 409 | 410 |
| | | | ALK1-Fc | 413 | 414 |
| | | B | ActRIIA-Fc | 451 | 452 |
| | | | ALK1-Fc | 463 | 464 |
| ActRIIA-Fc:ALK2-Fc | Electrostatic | A | ActRIIA-Fc | 118 | 120 |
| | | | ALK2-Fc | 136 | 138 |
| | | B | ActRIIA-Fc | 151 | 152 |
| | | | ALK2-Fc | 173 | 174 |
| | Hydrophobic | A | ActRIIA-Fc | 409 | 410 |
| | | | ALK2-Fc | 421 | 422 |
| | | B | ActRIIA-Fc | 451 | 452 |
| | | | ALK2-Fc | 465 | 466 |
| ActRIIA-Fc:ALK3-Fc | Electrostatic | A | ActRIIA-Fc | 118 | 120 |
| | | | ALK3-Fc | 115 | 117 |
| | | B | ActRIIA-Fc | 151 | 152 |
| | | | ALK3-Fc | 175 | 176 |
| | Hydrophobic | A | ActRIIA-Fc | 409 | 410 |
| | | | ALK3-Fc | 407 | 408 |
| | | B | ActRIIA-Fc | 451 | 452 |
| | | | ALK3-Fc | 467 | 468 |
| ActRIIA-Fc:ALK4-Fc | Electrostatic | A | ActRIIA-Fc | 118 | 120 |
| | | | ALK4-Fc | 104 | 106 |
| | | B | ActRIIA-Fc | 151 | 152 |
| | | | ALK4-Fc | 177 | 178 |
| | Hydrophobic | A | ActRIIA-Fc | 409 | 410 |
| | | | ALK4-Fc | 403 | 404 |
| | | B | ActRIIA-Fc | 451 | 452 |
| | | | ALK4-Fc | 469 | 470 |
| ActRIIA-Fc:ALK5-Fc | Electrostatic | A | ActRIIA-Fc | 118 | 120 |
| | | | ALK5-Fc | 139 | 141 |
| | | B | ActRIIA-Fc | 151 | 152 |
| | | | ALK5-Fc | 179 | 180 |
| | Hydrophobic | A | ActRIIA-Fc | 409 | 410 |
| | | | ALK5-Fc | 423 | 424 |
| | | B | ActRIIA-Fc | 451 | 452 |
| | | | ALK5-Fc | 471 | 472 |
| ActRIIA-Fc:ALK6-Fc | Electrostatic | A | ActRIIA-Fc | 118 | 120 |
| | | | ALK6-Fc | 142 | 144 |
| | | B | ActRIIA-Fc | 151 | 152 |
| | | | ALK6-Fc | 181 | 182 |
| | Hydrophobic | A | ActRIIA-Fc | 409 | 410 |
| | | | ALK6-Fc | 425 | 426 |
| | | B | ActRIIA-Fc | 451 | 452 |
| | | | ALK6-Fc | 473 | 474 |
| ActRIIA-Fc:ALK7-Fc | Electrostatic | A | ActRIIA-Fc | 118 | 120 |
| | | | ALK7-Fc | 112 | 114 |
| | | B | ActRIIA-Fc | 151 | 152 |
| | | | ALK7-Fc | 183 | 184 |
| | Hydrophobic | A | ActRIIA-Fc | 409 | 410 |
| | | | ALK7-Fc | 405 | 406 |
| | | B | ActRIIA-Fc | 451 | 452 |
| | | | ALK7-Fc | 475 | 476 |
| ActRIIB-Fc:ALK1-Fc | Electrostatic | A | ActRIIB-Fc | 100 | 102 |
| | | | ALK1-Fc | 124 | 126 |
| | | B | ActRIIB-Fc | 153 | 154 |
| | | | ALK1-Fc | 171 | 172 |
| | Hydrophobic | A | ActRIIB-Fc | 401 | 402 |
| | | | ALK1-Fc | 413 | 414 |
| | | B | ActRIIB-Fc | 453 | 454 |
| | | | ALK1-Fc | 463 | 464 |
| ActRIIB-Fc:ALK2-Fc | Electrostatic | A | ActRIIB-Fc | 100 | 102 |
| | | | ALK2-Fc | 136 | 138 |
| | | B | ActRIIB-Fc | 153 | 154 |
| | | | ALK2-Fc | 173 | 174 |
| | Hydrophobic | A | ActRIIB-Fc | 401 | 402 |
| | | | ALK2-Fc | 421 | 422 |
| | | B | ActRIIB-Fc | 453 | 454 |
| | | | ALK2-Fc | 465 | 466 |
| ActRIIB-Fc:ALK3-Fc | Electrostatic | A | ActRIIB-Fc | 100 | 102 |
| | | | ALK3-Fc | 115 | 117 |
| | | B | ActRIIB-Fc | 153 | 154 |
| | | | ALK3-Fc | 175 | 176 |
| | Hydrophobic | A | ActRIIB-Fc | 401 | 402 |
| | | | ALK3-Fc | 407 | 408 |
| | | B | ActRIIB-Fc | 453 | 454 |
| | | | ALK3-Fc | 467 | 468 |
| ActRIIB-Fc:ALK4-Fc | Electrostatic | A | ActRIIB-Fc | 100 | 102 |
| | | | ALK4-Fc | 104 | 106 |
| | | B | ActRIIB-Fc | 153 | 154 |
| | | | ALK4-Fc | 177 | 178 |
| | Hydrophobic | A | ActRIIB-Fc | 401 | 402 |
| | | | ALK4-Fc | 403 | 404 |
| | | B | ActRIIB-Fc | 453 | 454 |
| | | | ALK4-Fc | 469 | 470 |
| ActRIIB-Fc:ALK5-Fc | Electrostatic | A | ActRIIB-Fc | 100 | 102 |
| | | | ALK5-Fc | 139 | 141 |
| | | B | ActRIIB-Fc | 153 | 154 |
| | | | ALK5-Fc | 179 | 180 |
| | Hydrophobic | A | ActRIIB-Fc | 401 | 402 |
| | | | ALK5-Fc | 423 | 424 |
| | | B | ActRIIB-Fc | 453 | 454 |
| | | | ALK5-Fc | 471 | 472 |
| ActRIIB-Fc:ALK6-Fc | Electrostatic | A | ActRIIB-Fc | 100 | 102 |
| | | | ALK6-Fc | 142 | 144 |
| | | B | ActRIIB-Fc | 153 | 154 |
| | | | ALK6-Fc | 181 | 182 |
| | Hydrophobic | A | ActRIIB-Fc | 401 | 402 |
| | | | ALK6-Fc | 425 | 426 |
| | | B | ActRIIB-Fc | 453 | 454 |
| | | | ALK6-Fc | 473 | 474 |
| ActRIIB-Fc:ALK7-Fc | Electrostatic | A | ActRIIB-Fc | 100 | 102 |
| | | | ALK7-Fc | 112 | 114 |
| | | B | ActRIIB-Fc | 153 | 154 |
| | | | ALK7-Fc | 183 | 184 |
| | Hydrophobic | A | ActRIIB-Fc | 401 | 402 |
| | | | ALK7-Fc | 405 | 406 |
| | | B | ActRIIB-Fc | 453 | 454 |
| | | | ALK7-Fc | 475 | 476 |
| BMPRII-Fc:ALK1-Fc | Electrostatic | A | BMPRII-Fc | 121 | 123 |
| | | | ALK1-Fc | 124 | 126 |
| | | B | BMPRII-Fc | 155 | 156 |
| | | | ALK1-Fc | 171 | 172 |
| | Hydrophobic | A | BMPRII-Fc | 411 | 412 |
| | | | ALK1-Fc | 413 | 414 |
| | | B | BMPRII-Fc | 455 | 456 |
| | | | ALK1-Fc | 463 | 464 |
| BMPRII-Fc:ALK2-Fc | Electrostatic | A | BMPRII-Fc | 121 | 123 |
| | | | ALK2-Fc | 136 | 138 |
| | | B | BMPRII-Fc | 155 | 156 |
| | | | ALK2-Fc | 173 | 174 |
| | Hydrophobic | A | BMPRII-Fc | 411 | 412 |
| | | | ALK2-Fc | 421 | 422 |
| | | B | BMPRII-Fc | 455 | 456 |
| | | | ALK2-Fc | 465 | 466 |
| BMPRII-Fc:ALK3-Fc | Electrostatic | A | BMPRII-Fc | 121 | 123 |
| | | | ALK3-Fc | 115 | 117 |
| | | B | BMPRII-Fc | 155 | 156 |
| | | | ALK3-Fc | 175 | 176 |
| | Hydrophobic | A | BMPRII-Fc | 411 | 412 |
| | | | ALK3-Fc | 407 | 408 |
| | | B | BMPRII-Fc | 455 | 456 |
| | | | ALK3-Fc | 467 | 468 |
| BMPRII-Fc:ALK4-Fc | Electrostatic | A | BMPRII-Fc | 121 | 123 |
| | | | ALK4-Fc | 104 | 106 |
| | | B | BMPRII-Fc | 155 | 156 |
| | | | ALK4-Fc | 177 | 178 |
| | Hydrophobic | A | BMPRII-Fc | 411 | 412 |
| | | | ALK4-Fc | 403 | 404 |
| | | B | BMPRII-Fc | 455 | 456 |
| | | | ALK4-Fc | 469 | 470 |
| BMPRII-Fc:ALK5-Fc | Electrostatic | A | BMPRII-Fc | 121 | 123 |
| | | | ALK5-Fc | 139 | 141 |
| | | B | BMPRII-Fc | 155 | 156 |
| | | | ALK5-Fc | 179 | 180 |
| | Hydrophobic | A | BMPRII-Fc | 411 | 412 |
| | | | ALK5-Fc | 423 | 424 |
| | | B | BMPRII-Fc | 455 | 456 |
| | | | ALK5-Fc | 471 | 472 |
| BMPRII-Fc:ALK6-Fc | Electrostatic | A | BMPRII-Fc | 121 | 123 |
| | | | ALK6-Fc | 142 | 144 |
| | | B | BMPRII-Fc | 155 | 156 |
| | | | ALK6-Fc | 181 | 182 |
| | Hydrophobic | A | BMPRII-Fc | 411 | 412 |
| | | | ALK6-Fc | 425 | 426 |
| | | B | BMPRII-Fc | 455 | 456 |
| | | | ALK6-Fc | 473 | 474 |
| BMPRII-Fc:ALK7-Fc | Electrostatic | A | BMPRII-Fc | 121 | 123 |
| | | | ALK7-Fc | 112 | 114 |
| | | B | BMPRII-Fc | 155 | 156 |
| | | | ALK7-Fc | 183 | 184 |
| | Hydrophobic | A | BMPRII-Fc | 411 | 412 |
| | | | ALK7-Fc | 405 | 406 |
| | | B | BMPRII-Fc | 455 | 456 |
| | | | ALK7-Fc | 475 | 476 |
| MISRII-Fc:ALK1-Fc | Electrostatic | A | MISRII-Fc | 133 | 135 |
| | | | ALK1-Fc | 124 | 126 |
| | | B | MISRII-Fc | 161 | 162 |
| | | | ALK1-Fc | 171 | 172 |
| | Hydrophobic | A | MISRII-Fc | 419 | 420 |
| | | | ALK1-Fc | 413 | 414 |
| | | B | MISRII-Fc | 457 | 458 |
| | | | ALK1-Fc | 463 | 464 |
| MISRII-Fc:ALK2-Fc | Electrostatic | A | MISRII-Fc | 133 | 135 |
| | | | ALK2-Fc | 136 | 138 |
| | | B | MISRII-Fc | 161 | 162 |
| | | | ALK2-Fc | 173 | 174 |
| | Hydrophobic | A | MISRII-Fc | 419 | 420 |
| | | | ALK2-Fc | 421 | 422 |
| | | B | MISRII-Fc | 457 | 458 |
| | | | ALK2-Fc | 465 | 466 |
| MISRII-Fc:ALK3-Fc | Electrostatic | A | MISRII-Fc | 133 | 135 |
| | | | ALK3-Fc | 115 | 117 |
| | | B | MISRII-Fc | 161 | 162 |
| | | | ALK3-Fc | 175 | 176 |
| | Hydrophobic | A | MISRII-Fc | 419 | 420 |
| | | | ALK3-Fc | 407 | 408 |
| | | B | MISRII-Fc | 457 | 458 |
| | | | ALK3-Fc | 467 | 468 |
| MISRII-Fc:ALK4-Fc | Electrostatic | A | MISRII-Fc | 133 | 135 |
| | | | ALK4-Fc | 104 | 106 |
| | | B | MISRII-Fc | 161 | 162 |
| | | | ALK4-Fc | 177 | 178 |
| | Hydrophobic | A | MISRII-Fc | 419 | 420 |
| | | | ALK4-Fc | 403 | 404 |
| | | B | MISRII-Fc | 457 | 458 |
| | | | ALK4-Fc | 469 | 470 |
| MISRII-Fc:ALK5-Fc | Electrostatic | A | MISRII-Fc | 133 | 135 |
| | | | ALK5-Fc | 139 | 141 |
| | | B | MISRII-Fc | 161 | 162 |
| | | | ALK5-Fc | 179 | 180 |
| | Hydrophobic | A | MISRII-Fc | 419 | 420 |
| | | | ALK5-Fc | 423 | 424 |
| | | B | MISRII-Fc | 457 | 458 |
| | | | ALK5-Fc | 471 | 472 |
| MISRII-Fc:ALK6-Fc | Electrostatic | A | MISRII-Fc | 133 | 135 |
| | | | ALK6-Fc | 142 | 144 |
| | | B | MISRII-Fc | 161 | 162 |
| | | | ALK6-Fc | 181 | 182 |
| | Hydrophobic | A | MISRII-Fc | 419 | 420 |
| | | | ALK6-Fc | 425 | 426 |
| | | B | MISRII-Fc | 457 | 458 |
| | | | ALK6-Fc | 473 | 474 |
| MISRII-Fc:ALK7-Fc | Electrostatic | A | MISRII-Fc | 133 | 135 |
| | | | ALK7-Fc | 112 | 114 |
| | | B | MISRII-Fc | 161 | 162 |
| | | | ALK7-Fc | 183 | 184 |
| | Hydrophobic | A | MISRII-Fc | 419 | 420 |
| | | | ALK7-Fc | 405 | 406 |
| | | B | MISRII-Fc | 457 | 458 |
| | | | ALK7-Fc | 475 | 476 |
| TGFβRII_{SHORT}-Fc:ALK1-Fc | Electrostatic | A | TGFβRII_{SHORT}-Fc | 127 | 129 |
| | | | ALK1-Fc | 124 | 126 |
| | | B | TGFβRII_{SHORT}-Fc | 157 | 158 |
| | | | ALK1-Fc | 171 | 172 |
| | Hydrophobic | A | TGFβRII_{SHORT}-Fc | 415 | 416 |
| | | | ALK1-Fc | 413 | 414 |
| | | B | TGFβRII_{SHORT}-Fc | 459 | 460 |
| | | | ALK1-Fc | 463 | 464 |
| TGFβRII_{SHORT}-Fc:ALK2-Fc | Electrostatic | A | TGFβRII_{SHORT}-Fc | 127 | 129 |
| | | | ALK2-Fc | 136 | 138 |
| | | B | TGFβRII_{SHORT}-Fc | 157 | 158 |
| | | | ALK2-Fc | 173 | 174 |
| | Hydrophobic | A | TGFβRII_{SHORT}-Fc | 415 | 416 |
| | | | ALK2-Fc | 421 | 422 |
| | | B | TGFβRII_{SHORT}-Fc | 459 | 460 |
| | | | ALK2-Fc | 465 | 466 |
| TGFβRII_{SHORT}-Fc:ALK3-Fc | Electrostatic | A | TGFβRII_{SHORT}-Fc | 127 | 129 |
| | | | ALK3-Fc | 115 | 117 |
| | | B | TGFβRII_{SHORT}-Fc | 157 | 158 |
| | | | ALK3-Fc | 175 | 176 |
| | Hydrophobic | A | TGFβRII_{SHORT}-Fc | 415 | 416 |
| | | | ALK3-Fc | 407 | 408 |
| | | B | TGFβRII_{SHORT}-Fc | 459 | 460 |
| | | | ALK3-Fc | 467 | 468 |
| TGFβRII_{SHORT}-Fc:ALK4-Fc | Electrostatic | A | TGFβRII_{SHORT}-Fc | 127 | 129 |
| | | | ALK4-Fc | 104 | 106 |
| | | B | TGFβRII_{SHORT}-Fc | 157 | 158 |
| | | | ALK4-Fc | 177 | 178 |
| | Hydrophobic | A | TGFβRII_{SHORT}-Fc | 415 | 416 |
| | | | ALK4-Fc | 403 | 404 |
| | | B | TGFβRII_{SHORT}-Fc | 459 | 460 |
| | | | ALK4-Fc | 469 | 470 |
| TGFβRII_{SHORT}-Fc:ALK5-Fc | Electrostatic | A | TGFβRII_{SHORT}-Fc | 127 | 129 |
| | | | ALK5-Fc | 139 | 141 |
| | | B | TGFβRII_{SHORT}-Fc | 157 | 158 |
| | | | ALK5-Fc | 179 | 180 |
| | Hydrophobic | A | TGFβRII_{SHORT}-Fc | 415 | 416 |
| | | | ALK5-Fc | 423 | 424 |
| | | B | TGFβRII_{SHORT}-Fc | 459 | 460 |
| | | | ALK5-Fc | 471 | 472 |
| TGFβRII_{SHORT}-Fc:ALK6-Fc | Electrostatic | A | TGFβRII_{SHORT}-Fc | 127 | 129 |
| | | | ALK6-Fc | 142 | 144 |
| | | B | TGFβRII_{SHORT}-Fc | 157 | 158 |
| | | | ALK6-Fc | 181 | 182 |
| | Hydrophobic | A | TGFβRII_{SHORT}-Fc | 415 | 416 |
| | | | ALK6-Fc | 425 | 426 |
| | | B | TGFβRII_{SHORT}-Fc | 459 | 460 |
| | | | ALK6-Fc | 473 | 474 |
| TGFβRII_{SHORT}-Fc:ALK7-Fc | Electrostatic | A | TGFβRII_{SHORT}-Fc | 127 | 129 |
| | | | ALK7-Fc | 112 | 114 |
| | | B | TGFβRII_{SHORT}-Fc | 157 | 158 |
| | | | ALK7-Fc | 183 | 184 |
| | Hydrophobic | A | TGFβRII_{SHORT}-Fc | 415 | 416 |
| | | | ALK7-Fc | 405 | 406 |
| | | B | TGFβRII_{SHORT}-Fc | 459 | 460 |
| | | | ALK7-Fc | 475 | 476 |
| TGFβRII_{LONG}-Fc:ALK1-Fc | Electrostatic | A | TGFβRII_{LONG}-Fc | 130 | 132 |
| | | | ALK1-Fc | 124 | 126 |
| | | B | TGFβRII_{LONG}-Fc | 159 | 160 |
| | | | ALK1-Fc | 171 | 172 |
| | Hydrophobic | A | TGFβRII_{LONG}-Fc | 417 | 418 |
| | | | ALK1-Fc | 413 | 414 |
| | | B | TGFβRII_{LONG}-Fc | 461 | 462 |
| | | | ALK1-Fc | 463 | 464 |
| TGFβRII_{LONG}-Fc:ALK2-Fc | Electrostatic | A | TGFβRII_{LONG}-Fc | 130 | 132 |
| | | | ALK2-Fc | 136 | 138 |
| | | B | TGFβRII_{LONG}-Fc | 159 | 160 |
| | | | ALK2-Fc | 173 | 174 |
| | Hydrophobic | A | TGFβRII_{LONG}-Fc | 417 | 418 |
| | | | ALK2-Fc | 421 | 422 |
| | | B | TGFβRII_{LONG}-Fc | 461 | 462 |
| | | | ALK2-Fc | 465 | 466 |
| TGFβRII_{LONG}-Fc:ALK3-Fc | Electrostatic | A | TGFβRII_{LONG}-Fc | 130 | 132 |
| | | | ALK3-Fc | 115 | 117 |
| | | B | TGFβRII_{LONG}-Fc | 159 | 160 |
| | | | ALK3-Fc | 175 | 176 |
| | Hydrophobic | A | TGFβRII_{LONG}-Fc | 417 | 418 |
| | | | ALK3-Fc | 407 | 408 |
| | | B | TGFβRII_{LONG}-Fc | 461 | 462 |
| | | | ALK3-Fc | 467 | 468 |
| TGFβRII_{LONG}-Fc:ALK4-Fc | Electrostatic | A | TGFβRII_{LONG}-Fc | 130 | 132 |
| | | | ALK4-Fc | 104 | 106 |
| | | B | TGFβRII_{LONG}-Fc | 159 | 160 |
| | | | ALK4-Fc | 177 | 178 |
| | Hydrophobic | A | TGFβRII_{LONG}-Fc | 417 | 418 |
| | | | ALK4-Fc | 403 | 404 |
| | | B | TGFβRII_{LONG}-Fc | 461 | 462 |
| | | | ALK4-Fc | 469 | 470 |
| TGFβRII_{LONG}-Fc:ALK5-Fc | Electrostatic | A | TGFβRII_{LONG}-Fc | 130 | 132 |
| | | | ALK5-Fc | 139 | 141 |
| | | B | TGFβRII_{LONG}-Fc | 159 | 160 |
| | | | ALK5-Fc | 179 | 180 |
| | Hydrophobic | A | TGFβRII_{LONG}-Fc | 417 | 418 |
| | | | ALK5-Fc | 423 | 424 |
| | | B | TGFβRII_{LONG}-Fc | 461 | 462 |
| | | | ALK5-Fc | 471 | 472 |
| TGFβRII_{LONG}-Fc:ALK6-Fc | Electrostatic | A | TGFβRII_{LONG}-Fc | 130 | 132 |
| | | | ALK6-Fc | 142 | 144 |
| | | B | TGFβRII_{LONG}-Fc | 159 | 160 |
| | | | ALK6-Fc | 181 | 182 |
| | Hydrophobic | A | TGFβRII_{LONG}-Fc | 417 | 418 |
| | | | ALK6-Fc | 425 | 426 |
| | | B | TGFβRII_{LONG}-Fc | 461 | 462 |
| | | | ALK6-Fc | 473 | 474 |
| TGFβRII_{LONG}-Fc:ALK7-Fc | Electrostatic | A | TGFβRII_{LONG}-Fc | 130 | 132 |
| | | | ALK7-Fc | 112 | 114 |
| | | B | TGFβRII_{LONG}-Fc | 159 | 160 |
| | | | ALK7-Fc | 183 | 184 |
| | Hydrophobic | A | TGFβRII_{LONG}-Fc | 417 | 418 |
| | | | ALK7-Fc | 405 | 406 |
| | | B | TGFβRII_{LONG}-Fc | 461 | 462 |
| | | | ALK7-Fc | 475 | 476 |

In their entirety, these examples demonstrate that type I and type II receptor polypeptides, when placed in the context of a heteromeric complex, form novel binding pockets that exhibit altered selectivity relative to either type of homomeric complex, allowing the formation of novel protein agents for possible use as therapeutic agents.

1-225. (canceled)

226. A recombinant heteromultimer comprising an ALK7 fusion protein comprising an ALK7 domain and a heterologous domain, and an ActRIIB fusion protein comprising an ActRIIB domain and a heterologous domain.

227. The heteromultimer of claim 226, wherein the ActRIIB domain comprises an amino acid sequence that is at least 90% identical to amino acids 29-109 of SEQ ID NO: 1.

228. The heteromultimer of claim 226, wherein the ActRIIB domain comprises an amino acid sequence that is at least 90% identical to amino acids 25-131 of SEQ ID NO: 1.

229. The heteromultimer of claim 226, wherein the ActRIIB domain comprises an amino acid sequence that is at least 90% identical to a polypeptide that:
a) begins at any one of amino acids of 20-29 of SEQ ID NO: 1, and
b) ends at any one of amino acids 109-134 of SEQ ID NO: 1.

230. The heteromultimer of claim 226, wherein the ActRIIB fusion protein comprises an amino acid sequence that is at least 90% identical to any one of SEQ ID NOs: 100, 102, 153, 154, 401, 402, 453, and 454.

231. The heteromultimer of claim 229, wherein the ALK7 domain comprises an amino acid sequence that is at least 90% identical to amino acids 28-92 of SEQ ID Nos: 38, 305, or 309.

232. The heteromultimer of claim 229, wherein the ALK7 domain comprises an amino acid sequence that is at least 90% identical to amino acids 21-133 of SEQ ID NOs: 38, 305, or 309.

233. The heteromultimer of claim 229, wherein the ALK7 domain comprises an amino acid sequence that is at least 90% identical to a polypeptide that:
a) begins at any one of amino acids of 21-28 of SEQ ID NO: 38, 305, or 309, and
b) ends at any one of amino acids 92-133 of SEQ ID NO: 38, 305, or 309.

234. The heteromultimer of claim 226, wherein the fusion protein comprises an amino acid sequence that is at least 90% identical to any one of SEQ ID NOs: 112, 114, 183, 184, 405, 406, 475, and 476.

235-495. (canceled)

460-461.¹ (canceled)

498. The heteromultimer of claim 226, wherein the ALK7 domain comprises an amino acid sequence that is at least 97% identical to amino acids 28-92 of SEQ ID NOs: 38 or 305, and wherein the ActRIIB domain comprises an amino acid sequence that is at least 95% identical to amino acids 29-109 of SEQ ID NO: 1.

499. The heteromultimer of claim 226, wherein the ALK7 domain comprises an amino acid sequence that is at least 99% identical to amino acids 28-92 of SEQ ID NOs: 38 or 305, and wherein the ActRIIB domain comprises an amino acid sequence that is at least 99% identical to amino acids 29-109 of SEQ ID NO: 1.

500. The heteromultimer of claim 226, wherein the ALK7 domain comprises the amino acid sequence corresponding to amino acids 28-92 of SEQ ID NOs: 38 or 305, and wherein the ActRIIB domain comprises the amino acid sequence corresponding to amino acids 29-109 of SEQ ID NO: 1.

501. The heteromultimer of claim 498, wherein the heterologous domain of the ALK7 fusion protein and the heterologous domain of the ActRIIB fusion protein each comprises an Fc immunoglobulin domain, wherein the Fc immunoglobulin domain is an IgG1 immunoglobulin domain.

502. The heteromultimer of claim 499, wherein the heterologous domain of the ALK7 polypeptide and the heterologous domain of the ActRIIB polypeptide each comprises an Fc immunoglobulin domain, wherein the Fc immunoglobulin domain is an IgG1 immunoglobulin domain.

503. The heteromultimer of claim 500, wherein the heterologous domain of the ALK7 polypeptide and the heterologous domain of the ActRIIB polypeptide each comprises an Fc immunoglobulin domain, wherein the Fc immunoglobulin domain is an IgG1 immunoglobulin domain.

504. The heteromultimer of claim 498, wherein a linker domain is positioned between the ALK7 domain and the heterologous domain and/or a linker domain is positioned between the ActRIIB domain and the heterologous domain.

505. The heteromultimer of claim 499, wherein a linker domain is positioned between the ALK7 domain and the heterologous domain and/or a linker domain is positioned between the ActRIIB domain and the heterologous domain.

506. The heteromultimer of claim 500, wherein a linker domain is positioned between the ALK7 domain and the heterologous domain and/or a linker domain is positioned between the ActRIIB domain and the heterologous domain.

507. A method of treating a muscle disorder and/or neuromuscular disorder in a subject in need thereof, comprising administering a therapeutically effective amount of the heteromultimer of claim 226 to the subject.


SEQUENCE LISTING (509 sequences)

SEQ ID NO: 1 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 2 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 3 - protein, 100 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 4 - protein, 512 aa, Homo sapiens
MTAPWVALALLWGSLCAGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 5 - protein, 115 aa, Homo sapiens
GRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 6 - protein, 100 aa, Homo sapiens
GRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 7 - DNA, 1536 bp, Homo sapiens
  source 1..1536: mol_type = unassigned DNA
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatc

SEQ ID NO: 8 - DNA, 345 bp, Homo sapiens
  source 1..345: mol_type = unassigned DNA
gggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccacc

SEQ ID NO: 9 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 10 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 11 - protein, 100 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 12 - DNA, 1539 bp, Homo sapiens
  source 1..1539: mol_type = unassigned DNA
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 13 - DNA, 345 bp, Homo sapiens
  source 1..345: mol_type = unassigned DNA
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 14 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 15 - protein, 97 aa, Homo sapiens
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQ

SEQ ID NO: 16 - DNA, 1509 bp, Homo sapiens
  source 1..1509: mol_type = unassigned DNA
atgaccttgggctcccccaggaaaggccttctgatgctgctgatggccttggtgacccagggagaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccagctggccctgatcctgggccccgtgctggccttgctggccctggtggccctgggtgtcctgggcctgtggcatgtccgacggaggcaggagaagcagcgtggcctgcacagcgagctgggagagtccagtctcatcctgaaagcatctgagcagggcgacagcatgttgggggacctcctggacagtgactgcaccacagggagtggctcagggctccccttcctggtgcagaggacagtggcacggcaggttgccttggtggagtgtgtgggaaaaggccgctatggcgaagtgtggcggggcttgtggcacggtgagagtgtggccgtcaagatcttctcctcgagggatgaacagtcctggttccgggagactgagatctataacacagtgttgctcagacacgacaacatcctaggcttcatcgcctcagacatgacctcccgcaactcgagcacgcagctgtggctcatcacgcactaccacgagcacggctccctctacgactttctgcagagacagacgctggagccccatctggctctgaggctagctgtgtccgcggcatgcggcctggcgcacctgcacgtggagatcttcggtacacagggcaaaccagccattgcccaccgcgacttcaagagccgcaatgtgctggtcaagagcaacctgcagtgttgcatcgccgacctgggcctggctgtgatgcactcacagggcagcgattacctggacatcggcaacaacccgagagtgggcaccaagcggtacatggcacccgaggtgctggacgagcagatccgcacggactgctttgagtcctacaagtggactgacatctgggcctttggcctggtgctgtgggagattgcccgccggaccatcgtgaatggcatcgtggaggactatagaccacccttctatgatgtggtgcccaatgaccccagctttgaggacatgaagaaggtggtgtgtgtggatcagcagacccccaccatccctaaccggctggctgcagacccggtcctctcaggcctagctcagatgatgcgggagtgctggtacccaaacccctctgcccgactcaccgcgctgcggatcaagaagacactacaaaaaattagcaacagtccagagaagcctaaagtgattcaa

SEQ ID NO: 17 - DNA, 291 bp, Homo sapiens
  source 1..291: mol_type = unassigned DNA
gaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccag

SEQ ID NO: 18 - protein, 509 aa, Homo sapiens
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 19 - protein, 103 aa, Homo sapiens
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLE

SEQ ID NO: 20 - DNA, 1527 bp, Homo sapiens
  source 1..1527: mol_type = unassigned DNA
atggtagatggagtgatgattcttcctgtgcttatcatgattgctctcccctcccctagtatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagccgtggagtgctgccaaggggactggtgtaacaggaacatcacggcccagctgcccactaaaggaaaatccttccctggaacacagaatttccacttggaggttggcctcattattctctctgtagtgttcgcagtatgtcttttagcctgcctgctgggagttgctctccgaaaatttaaaaggcgcaaccaagaacgcctcaatccccgagacgtggagtatggcactatcgaagggctcatcaccaccaatgttggagacagcactttagcagatttattggatcattcgtgtacatcaggaagtggctctggtcttccttttctggtacaaagaacagtggctcgccagattacactgttggagtgtgtcgggaaaggcaggtatggtgaggtgtggaggggcagctggcaaggggagaatgttgccgtgaagatcttctcctcccgtgatgagaagtcatggttcagggaaacggaattgtacaacactgtgatgctgaggcatgaaaatatcttaggtttcattgcttcagacatgacatcaagacactccagtacccagctgtggttaattacacattatcatgaaatgggatcgttgtacgactatcttcagcttactactctggatacagttagctgccttcgaatagtgctgtccatagctagtggtcttgcacatttgcacatagagatatttgggacccaagggaaaccagccattgcccatcgagatttaaagagcaaaaatattctggttaagaagaatggacagtgttgcatagcagatttgggcctggcagtcatgcattcccagagcaccaatcagcttgatgtggggaacaatccccgtgtgggcaccaagcgctacatggcccccgaagttctagatgaaaccatccaggtggattgtttcgattcttataaaagggtcgatatttgggcctttggacttgttttgtgggaagtggccaggcggatggtgagcaatggtatagtggaggattacaagccaccgttctacgatgtggttcccaatgacccaagttttgaagatatgaggaaggtagtctgtgtggatcaacaaaggccaaacatacccaacagatggttctcagacccgacattaacctctctggccaagctaatgaaagaatgctggtatcaaaatccatccgcaagactcacagcactgcgtatcaaaaagactttgaccaaaattgataattccctcgacaaattgaaaactgactgt

SEQ ID NO: 21 - DNA, 309 bp, Homo sapiens
  source 1..309: mol_type = unassigned DNA
atggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagccgtggagtgctgccaaggggactggtgtaacaggaacatcacggcccagctgcccactaaaggaaaatccttccctggaacacagaatttccacttggag

SEQ ID NO: 22 - protein, 532 aa, Homo sapiens
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 23 - protein, 129 aa, Homo sapiens
QNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIR

SEQ ID NO: 24 - DNA, 1596 bp, Homo sapiens
  source 1..1596: mol_type = unassigned DNA
atgcctcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatc

SEQ ID NO: 25 - DNA, 387 bp, Homo sapiens
  source 1..387: mol_type = unassigned DNA
cagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcga

SEQ ID NO: 26 - protein, 505 aa, Homo sapiens
MAESAGASSFFPLVVLLLAGSGGSGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVELVGIIAGPVFLLFLIIIIVFLVINYHQRVYHNRQRLDMEDPSCEMCLSKDKTLQDLVYDLSTSGSGSGLPLFVQRTVARTIVLQEIIGKGRFGEVWRGRWRGGDVAVKIFSSREERSWFREAEIYQTVMLRHENILGFIAADNKDNGTWTQLWLVSDYHEHGSLFDYLNRYTVTIEGMIKLALSAASGLAHLHMEIVGTQGKPGIAHRDLKSKNILVKKNGMCAIADLGLAVRHDAVTDTIDIAPNQRVGTKRYMAPEVLDETINMKHFDSFKCADIYALGLVYWEIARRCNSGGVHEEYQLPYYDLVPSDPSIEEMRKVVCDQKLRPNIPNWWQSYEALRVMGKMMRECWYANGAARLTALRIKKTLSQLSVQEDVKI

SEQ ID NO: 27 - protein, 103 aa, Homo sapiens
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVE

SEQ ID NO: 28 - DNA, 1515 bp, Homo sapiens
  source 1..1515: mol_type = unassigned DNA
atggcggagtcggccggagcctcctccttcttcccccttgttgtcctcctgctcgccggcagcggcgggtccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtaggcatcatcgccggcccggtgttcctcctgttcctcatcatcatcattgttttccttgtcattaactatcatcagcgtgtctatcacaaccgccagagactggacatggaagatccctcatgtgagatgtgtctctccaaagacaagacgctccaggatcttgtctacgatctctccacctcagggtctggctcagggttacccctctttgtccagcgcacagtggcccgaaccatcgttttacaagagattattggcaagggtcggtttggggaagtatggcggggccgctggaggggtggtgatgtggctgtgaaaatattctcttctcgtgaagaacggtcttggttcagggaagcagagatataccagacggtcatgctgcgccatgaaaacatccttggatttattgctgctgacaataaagataatggcacctggacacagctgtggcttgtttctgactatcatgagcacgggtccctgtttgattatctgaaccggtacacagtgacaattgaggggatgattaagctggccttgtctgctgctagtgggctggcacacctgcacatggagatcgtgggcacccaagggaagcctggaattgctcatcgagacttaaagtcaaagaacattctggtgaagaaaaatggcatgtgtgccatagcagacctgggcctggctgtccgtcatgatgcagtcactgacaccattgacattgccccgaatcagagggtggggaccaaacgatacatggcccctgaagtacttgatgaaaccattaatatgaaacactttgactcctttaaatgtgctgatatttatgccctcgggcttgtatattgggagattgctcgaagatgcaattctggaggagtccatgaagaatatcagctgccatattacgacttagtgccctctgacccttccattgaggaaatgcgaaaggttgtatgtgatcagaagctgcgtcccaacatccccaactggtggcagagttatgaggcactgcgggtgatggggaagatgatgcgagagtgttggtatgccaacggcgcagcccgcctgacggccctgcgcatcaagaagaccctctcccagctcagcgtgcaggaagacgtgaagatc

SEQ ID NO: 29 - DNA, 309 bp, Homo sapiens
  source 1..309: mol_type = unassigned DNA
tccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggag

SEQ ID NO: 30 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 31 - protein, 102 aa, Homo sapiens
AALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 32 - DNA, 1509 bp, Homo sapiens
  source 1..1509: mol_type = unassigned DNA
atggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaaccgcactgtcattcaccatcgagtgccaaatgaagaggacccttcattagatcgcccttttatttcagagggtactacgttgaaagacttaatttatgatatgacaacgtcaggttctggctcaggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatg

SEQ ID NO: 33 - DNA, 306 bp, Homo sapiens
  source 1..306: mol_type = unassigned DNA
gcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactg

SEQ ID NO: 34 - protein, 502 aa, Homo sapiens
MLLRSAGKLNVGTKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRALLISVTVCSLLLVLIILFCYFRYKRQETRPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDAKSMLKLAYSSVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKL

SEQ ID NO: 35 - protein, 113 aa, Homo sapiens
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHR

SEQ ID NO: 36 - DNA, 1506 bp, Homo sapiens
  source 1..1506: mol_type = unassigned DNA
atgcttttgcgaagtgcaggaaaattaaatgtgggcaccaagaaagaggatggtgagagtacagcccccaccccccgtccaaaggtcttgcgttgtaaatgccaccaccattgtccagaagactcagtcaacaatatttgcagcacagacggatattgtttcacgatgatagaagaggatgactctgggttgcctgtggtcacttctggttgcctaggactagaaggctcagattttcagtgtcgggacactcccattcctcatcaaagaagatcaattgaatgctgcacagaaaggaacgaatgtaataaagacctacaccctacactgcctccattgaaaaacagagattttgttgatggacctatacaccacagggctttacttatatctgtgactgtctgtagtttgctcttggtccttatcatattattttgttacttccggtataaaagacaagaaaccagacctcgatacagcattgggttagaacaggatgaaacttacattcctcctggagaatccctgagagacttaattgagcagtctcagagctcaggaagtggatcaggcctccctctgctggtccaaaggactatagctaagcagattcagatggtgaaacagattggaaaaggtcgctatggggaagtttggatgggaaagtggcgtggcgaaaaggtagctgtgaaagtgttcttcaccacagaggaagccagctggttcagagagacagaaatatatcagacagtgttgatgaggcatgaaaacattttgggtttcattgctgcagatatcaaagggacagggtcctggacccagttgtacctaatcacagactatcatgaaaatggttccctttatgattatctgaagtccaccaccctagacgctaaatcaatgctgaagttagcctactcttctgtcagtggcttatgtcatttacacacagaaatctttagtactcaaggcaaaccagcaattgcccatcgagatctgaaaagtaaaaacattctggtgaagaaaaatggaacttgctgtattgctgacctgggcctggctgttaaatttattagtgatacaaatgaagttgacataccacctaacactcgagttggcaccaaacgctatatgcctccagaagtgttggacgagagcttgaacagaaatcacttccagtcttacatcatggctgacatgtatagttttggcctcatcctttgggaggttgctaggagatgtgtatcaggaggtatagtggaagaataccagcttccttatcatgacctagtgcccagtgacccctcttatgaggacatgagggagattgtgtgcatcaagaagttacgcccctcattcccaaaccggtggagcagtgatgagtgtctaaggcagatgggaaaactcatgacagaatgctgggctcacaatcctgcatcaaggctgacagccctgcgggttaagaaaacacttgccaaaatgtcagagtcccaggacattaaactc

SEQ ID NO: 37 - DNA, 339 bp, Homo sapiens
  source 1..339: mol_type = unassigned DNA
aagaaagaggatggtgagagtacagcccccaccccccgtccaaaggtcttgcgttgtaaatgccaccaccattgtccagaagactcagtcaacaatatttgcagcacagacggatattgtttcacgatgatagaagaggatgactctgggttgcctgtggtcacttctggttgcctaggactagaaggctcagattttcagtgtcgggacactcccattcctcatcaaagaagatcaattgaatgctgcacagaaaggaacgaatgtaataaagacctacaccctacactgcctccattgaaaaacagagattttgttgatggacctatacaccacagg

SEQ ID NO: 38 - protein, 493 aa, Homo sapiens
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTASPNAPKLGPMELAIIITVPVCLLSIAAMLTVWACQGRQCSYRKKKRPNVEEPLSECNLVNAGKTLKDLIYDVTASGSGSGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 39 - protein, 93 aa, Homo sapiens
ELSPGLKCVCLLCDSSNFTCQTEGACWASVMLTNGKEQVIKSCVSLPELNAQVFCHSSNNVTKTECCFTDFCNNITLHLPTASPNAPKLGPME

SEQ ID NO: 40 - DNA, 1479 bp, Homo sapiens
  source 1..1479: mol_type = unassigned DNA
atgacccgggcgctctgctcagcgctccgccaggctctcctgctgctcgcagcggccgccgagctctcgccaggactgaagtgtgtatgtcttttgtgtgattcttcaaactttacctgccaaacagaaggagcatgttgggcatcagtcatgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacagcatcaccaaatgccccaaaacttggacccatggagctggccatcattattactgtgcctgtttgcctcctgtccatagctgcgatgctgacagtatgggcatgccagggtcgacagtgctcctacaggaagaaaaagagaccaaatgtggaggaaccactctctgagtgcaatctggtaaatgctggaaaaactctgaaagatctgatttatgatgtgaccgcctctggatctggctctggtctacctctgttggttcaaaggacaattgcaaggacgattgtgcttcaggaaatagtaggaaaaggtagatttggtgaggtgtggcatggaagatggtgtggggaagatgtggctgtgaaaatattctcctccagagatgaaagatcttggtttcgtgaggcagaaatttaccagacggtcatgctgcgacatgaaaacatccttggtttcattgctgctgacaacaaagataatggaacttggactcaactttggctggtatctgaatatcatgaacagggctccttatatgactatttgaatagaaatatagtgaccgtggctggaatgatcaagctggcgctctcaattgctagtggtctggcacaccttcatatggagattgttggtacacaaggtaaacctgctattgctcatcgagacataaaatcaaagaatatcttagtgaaaaagtgtgaaacttgtgccatagcggacttagggttggctgtgaagcatgattcaatactgaacactatcgacatacctcagaatcctaaagtgggaaccaagaggtatatggctcctgaaatgcttgatgatacaatgaatgtgaatatctttgagtccttcaaacgagctgacatctattctgttggtctggtttactgggaaatagcccggaggtgttcagtcggaggaattgttgaggagtaccaattgccttattatgacatggtgccttcagatccctcgatagaggaaatgagaaaggttgtttgtgaccagaagtttcgaccaagtatcccaaaccagtggcaaagttgtgaagcactccgagtcatggggagaataatgcgtgagtgttggtatgccaacggagcggcccgcctaactgctcttcgtattaagaagactatatctcaactttgtgtcaaagaagactgcaaagcc

SEQ ID NO: 41 - DNA, 279 bp, Homo sapiens
  source 1..279: mol_type = unassigned DNA
gagctctcgccaggactgaagtgtgtatgtcttttgtgtgattcttcaaactttacctgccaaacagaaggagcatgttgggcatcagtcatgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacagcatcaccaaatgccccaaaacttggacccatggag

SEQ ID NO: 42 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 43 - protein, 144 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 44 - DNA, 1701 bp, Homo sapiens
  source 1..1701: mol_type = unassigned DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaa

SEQ ID NO: 45 - DNA, 432 bp, Homo sapiens
  source 1..432: mol_type = unassigned DNA
acgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaa

SEQ ID NO: 46 - protein, 1038 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETNLHTTNVAQSIGPTPVCLQLTEEDLETNKLDPKEVDKNLKESSDENLMEHSLKQFSGPDPLSSTSSSLLYPLIKLAVEATGQQDFTQTANGQACLIPDVLPTQIYPLPKQQNLPKRPTSLPLNTKNSTKEPRLKFGSKHKSNLKQVETGVAKMNTINAAEPHVVTVTMNGVAGRNHSVNSHAATTQYANGTVLSGQTTNIVTHRAQEMLQNQFIGEDTRLNINSSPDEHEPLLRREQQAGHDEGVLDRLVDRRERPLEGGRTNSNNNNSNPCSEQDVLAQGVPSTAADPGPSKPRRAQRPNSLDLSATNVLDGSSIQIGESTQDGKSGSGEKIKKRVKTPYSLKRWRPSTWVISTESLDCEVNNNGSNRAVHSKSSTAVYLAEGGTATTMVSKDIGMNCL

SEQ ID NO: 47 - protein, 124 aa, Homo sapiens [sequence omitted]

SEQ ID NO: 48 - DNA, 3114 bp, Homo sapiens
  source 1..3114: mol_type = unassigned DNA
atgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgcaacctgtcacataataggcgtgtgccaaaaattggtccttatccagattattcttcctcctcatacattgaagactctatccatcatactgacagcatcgtgaagaatatttcctctgagcattctatgtccagcacacctttgactataggggaaaaaaaccgaaattcaattaactatgaacgacagcaagcacaagctcgaatccccagccctgaaacaagtgtcaccagcctctccaccaacacaacaaccacaaacaccacaggactcacgccaagtactggcatgactactatatctgagatgccatacccagatgaaacaaatctgcataccacaaatgttgcacagtcaattgggccaacccctgtctgcttacagctgacagaagaagacttggaaaccaacaagctagacccaaaagaagttgataagaacctcaaggaaagctctgatgagaatctcatggagcactctcttaaacagttcagtggcccagacccactgagcagtactagttctagcttgctttacccactcataaaacttgcagtagaagcaactggacagcaggacttcacacagactgcaaatggccaagcatgtttgattcctgatgttctgcctactcagatctatcctctccccaagcagcagaaccttcccaagagacctactagtttgcctttgaacaccaaaaattcaacaaaagagccccggctaaaatttggcagcaagcacaaatcaaacttgaaacaagtcgaaactggagttgccaagatgaatacaatcaatgcagcagaacctcatgtggtgacagtcaccatgaatggtgtggcaggtagaaaccacagtgttaactcccatgctgccacaacccaatatgccaatgggacagtactatctggccaaacaaccaacatagtgacacatagggcccaagaaatgttgcagaatcagtttattggtgaggacacccggctgaatattaattccagtcctgatgagcatgagcctttactgagacgagagcaacaagctggccatgatgaaggtgttctggatcgtcttgtggacaggagggaacggccactagaaggtggccgaactaattccaataacaacaacagcaatccatgttcagaacaagatgttcttgcacagggtgttccaagcacagcagcagatcctgggccatcaaagcccagaagagcacagaggcctaattctctggatctttcagccacaaatgtcctggatggcagcagtatacagataggtgagtcaacacaagatggcaaatcaggatcaggtgaaaagatcaagaaacgtgtgaaaactccctattctcttaagcggtggcgcccctccacctgggtcatctccactgaatcgctggactgtgaagtcaacaataatggcagtaacagggcagttcattccaaatccagcactgctgtttaccttgcagaaggaggcactgctacaaccatggtgtctaaagatataggaatgaactgtctg

SEQ ID NO: 49 - DNA, 372 bp, Homo sapiens
  source 1..372: mol_type = unassigned DNA
tcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagaca

SEQ ID NO: 50 - protein, 573 aa, Homo sapiens
MLGSLGLWALLPTAVEAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALVLLGLFLLLLLLLGSIILALLQRKNYRVRGEPVPEPRPDSGRDWSVELQELPELCFSQVIREGGHAVVWAGQLQGKLVAIKAFPPRSVAQFQAERALYELPGLQHDHIVRFITASRGGPGRLLSGPLLVLELHPKGSLCHYLTQYTSDWGSSLRMALSLAQGLAFLHEERWQNGQYKPGIAHRDLSSQNVLIREDGSCAIGDLGLALVLPGLTQPPAWTPTQPQGPAAIMEAGTQRYMAPELLDKTLDLQDWGMALRRADIYSLALLLWEILSRCPDLRPDSSPPPFQLAYEAELGNTPTSDELWALAVQERRRPYIPSTWRCFATDPDGLRELLEDCWDADPEARLTAECVQQRLAALAHPQESHPFPESCPRGCPPLCPEDCTSIPAPTILPCRPQRSACHFSVQQGPCSRNPQPACTLSPV

SEQ ID NO: 51 - protein, 132 aa, Homo sapiens [sequence omitted]

SEQ ID NO: 52 - DNA, 1719 bp, Homo sapiens
  source 1..1719: mol_type = unassigned DNA
atgctagggtctttggggctttgggcattacttcccacagctgtggaagcacccccaaacaggcgaacctgtgtgttctttgaggcccctggagtgcggggaagcacaaagacactgggagagctgctagatacaggcacagagctccccagagctatccgctgcctctacagccgctgctgctttgggatctggaacctgacccaagaccgggcacaggtggaaatgcaaggatgccgagacagtgatgagccaggctgtgagtccctccactgtgacccaagtccccgagcccaccccagccctggctccactctcttcacctgctcctgtggcactgacttctgcaatgccaattacagccatctgcctcctccagggagccctgggactcctggctcccagggtccccaggctgccccaggtgagtccatctggatggcactggtgctgctggggctgttcctcctcctcctgctgctgctgggcagcatcatcttggccctgctacagcgaaagaactacagagtgcgaggtgagccagtgccagagccaaggccagactcaggcagggactggagtgtggagctgcaggagctgcctgagctgtgtttctcccaggtaatccgggaaggaggtcatgcagtggtttgggccgggcagctgcaaggaaaactggttgccatcaaggccttcccaccgaggtctgtggctcagttccaagctgagagagcattgtacgaacttccaggcctacagcacgaccacattgtccgatttatcactgccagccgggggggtcctggccgcctgctctctgggcccctgctggtactggaactgcatcccaagggctccctgtgccactacttgacccagtacaccagtgactggggaagttccctgcggatggcactgtccctggcccagggcctggcatttctccatgaggagcgctggcagaatggccaatataaaccaggtattgcccaccgagatctgagcagccagaatgtgctcattcgggaagatggatcgtgtgccattggagacctgggccttgccttggtgctccctggcctcactcagccccctgcctggacccctactcaaccacaaggcccagctgccatcatggaagctggcacccagaggtacatggcaccagagctcttggacaagactctggacctacaggattggggcatggccctccgacgagctgatatttactctttggctctgctcctgtgggagatactgagccgctgcccagatttgaggcctgacagcagtccaccacccttccaactggcctatgaggcagaactgggcaatacccctacctctgatgagctatgggccttggcagtgcaggagaggaggcgtccctacatcccatccacctggcgctgctttgccacagaccctgatgggctgagggagctcctagaagactgttgggatgcagacccagaagcacggctgacagctgagtgtgtacagcagcgcctggctgccttggcccatcctcaagagagccacccctttccagagagctgtccacgtggctgcccacctctctgcccagaagactgtacttcaattcctgcccctaccatcctcccctgtaggcctcagcggagtgcctgccacttcagcgttcagcaaggcccttgttccaggaatcctcagcctgcctgtaccctttctcctgtg

SEQ ID NO: 53 - DNA, 396 bp, Homo sapiens
  source 1..396: mol_type = unassigned DNA
cccccaaacaggcgaacctgtgtgttctttgaggcccctggagtgcggggaagcacaaagacactgggagagctgctagatacaggcacagagctccccagagctatccgctgcctctacagccgctgctgctttgggatctggaacctgacccaagaccgggcacaggtggaaatgcaaggatgccgagacagtgatgagccaggctgtgagtccctccactgtgacccaagtccccgagcccaccccagccctggctccactctcttcacctgctcctgtggcactgacttctgcaatgccaattacagccatctgcctcctccagggagccctgggactcctggctcccagggtccccaggctgccccaggtgagtccatctggatggcactg

SEQ ID NO: 54-58 - skipped (5 entries)

SEQ ID NO: 59 - protein, 4 aa, synthetic construct
  REGION 1..4: note = Description of Artificial Sequence: Synthetic peptide
GGGG

SEQ ID NO: 60 - protein, 5 aa, synthetic construct
  REGION 1..5: note = Description of Artificial Sequence: Synthetic peptide
TGGGG

SEQ ID NO: 61 - protein, 5 aa, synthetic construct
  REGION 1..5: note = Description of Artificial Sequence: Synthetic peptide
SGGGG

SEQ ID NO: 62 - protein, 4 aa, synthetic construct
  REGION 1..4: note = Description of Artificial Sequence: Synthetic peptide
TGGG

SEQ ID NO: 63 - protein, 4 aa, synthetic construct
  REGION 1..4: note = Description of Artificial Sequence: Synthetic peptide
SGGG

SEQ ID NO: 64-66 - skipped (3 entries)

SEQ ID NO: 67 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 68 - protein, 169 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 69 - DNA, 1776 bp, Homo sapiens
  source 1..1776: mol_type = unassigned DNA
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaa

SEQ ID NO: 70 - DNA, 507 bp, Homo sapiens
  source 1..507: mol_type = unassigned DNA
acgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaa

SEQ ID NO: 71 - protein, 530 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERR

SEQ ID NO: 72 - protein, 124 aa, Homo sapiens [sequence omitted]

SEQ ID NO: 73 - DNA, 1590 bp, Homo sapiens
  source 1..1590: mol_type = unassigned DNA
atgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgtagg

SEQ ID NO: 74 - DNA, 372 bp, Homo sapiens; identical to SEQ ID NO: 49
  source 1..372: mol_type = unassigned DNA

SEQ ID NO: 75 - protein, 478 aa, Homo sapiens
MLGSLGLWALLPTAVEAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALVLLGLFLLLLLLLGSIILALLQRKNYRVRGEPVPEPRPDSGRDWSVELQELPELCFSQVIREGGHAVVWAGQLQGKLVAIKAFPPRSVAQFQAERALYELPGLQHDHIVRFITASRGGPGRLLSGPLLVLELHPKGSLCHYLTQYTSDWGSSLRMALSLAQGLAFLHEERWQNGQYKPGIAHRDLSSQNVLIREDGSCAIGDLGLALVLPGLTQPPAWTPTQPQGPAAIMEAGTQRYMAPELLDKTLDLQDWGMALRRADIYSLALLLWEILSRCPDLRPAVHHPSNWPMRQNWAIPLPLMSYGPWQCRRGGVPTSHPPGAALPQTLMG

SEQ ID NO: 76 - protein, 132 aa, Homo sapiens [sequence omitted]

SEQ ID NO: 77 - DNA, 1434 bp, Homo sapiens
  source 1..1434: mol_type = unassigned DNA
atgctagggtctttggggctttgggcattacttcccacagctgtggaagcacccccaaacaggcgaacctgtgtgttctttgaggcccctggagtgcggggaagcacaaagacactgggagagctgctagatacaggcacagagctccccagagctatccgctgcctctacagccgctgctgctttgggatctggaacctgacccaagaccgggcacaggtggaaatgcaaggatgccgagacagtgatgagccaggctgtgagtccctccactgtgacccaagtccccgagcccaccccagccctggctccactctcttcacctgctcctgtggcactgacttctgcaatgccaattacagccatctgcctcctccagggagccctgggactcctggctcccagggtccccaggctgccccaggtgagtccatctggatggcactggtgctgctggggctgttcctcctcctcctgctgctgctgggcagcatcatcttggccctgctacagcgaaagaactacagagtgcgaggtgagccagtgccagagccaaggccagactcaggcagggactggagtgtggagctgcaggagctgcctgagctgtgtttctcccaggtaatccgggaaggaggtcatgcagtggtttgggccgggcagctgcaaggaaaactggttgccatcaaggccttcccaccgaggtctgtggctcagttccaagctgagagagcattgtacgaacttccaggcctacagcacgaccacattgtccgatttatcactgccagccgggggggtcctggccgcctgctctctgggcccctgctggtactggaactgcatcccaagggctccctgtgccactacttgacccagtacaccagtgactggggaagttccctgcggatggcactgtccctggcccagggcctggcatttctccatgaggagcgctggcagaatggccaatataaaccaggtattgcccaccgagatctgagcagccagaatgtgctcattcgggaagatggatcgtgtgccattggagacctgggccttgccttggtgctccctggcctcactcagccccctgcctggacccctactcaaccacaaggcccagctgccatcatggaagctggcacccagaggtacatggcaccagagctcttggacaagactctggacctacaggattggggcatggccctccgacgagctgatatttactctttggctctgctcctgtgggagatactgagccgctgcccagatttgaggcctgcagtccaccacccttccaactggcctatgaggcagaactgggcaatacccctacctctgatgagctatgggccttggcagtgcaggagaggaggcgtccctacatcccatccacctggcgctgctttgccacagaccctgatgggc

SEQ ID NO: 78 - DNA, 396 bp, Homo sapiens; identical to SEQ ID NO: 53
  source 1..396: mol_type = unassigned DNA

SEQ ID NO: 79 - protein, 478 aa, Homo sapiens
MLGSLGLWALLPTAVEAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALVLLGLFLLLLLLLGSIILALLQRKNYRVRGEPVPEPRPDSGRDWSVELQELPELCFSQVIREGGHAVVWAGQLQGKLVAIKAFPPRSVAQFQAERALYELPGLQHDHIVRFITASRGGPGRLLSGPLLVLELHPKGSLCHYLTQYTSDWGSSLRMALSLAQGLAFLHEERWQNGQYKPGIAHRDLSSQNVLIREDGSCAIGDLGLALVLPGLTQPPAWTPTQPQGPAAIMEDPDGLRELLEDCWDADPEARLTAECVQQRLAALAHPQESHPFPESCPRGCPPLCPEDCTSIPAPTILPCRPQRSACHFSVQQGPCSRNPQPACTLSPV

SEQ ID NO: 80 - protein, 132 aa, Homo sapiens [sequence omitted]

SEQ ID NO: 81 - DNA, 1434 bp, Homo sapiens
  source 1..1434: mol_type = unassigned DNA
atgctagggtctttggggctttgggcattacttcccacagctgtggaagcacccccaaacaggcgaacctgtgtgttctttgaggcccctggagtgcggggaagcacaaagacactgggagagctgctagatacaggcacagagctccccagagctatccgctgcctctacagccgctgctgctttgggatctggaacctgacccaagaccgggcacaggtggaaatgcaaggatgccgagacagtgatgagccaggctgtgagtccctccactgtgacccaagtccccgagcccaccccagccctggctccactctcttcacctgctcctgtggcactgacttctgcaatgccaattacagccatctgcctcctccagggagccctgggactcctggctcccagggtccccaggctgccccaggtgagtccatctggatggcactggtgctgctggggctgttcctcctcctcctgctgctgctgggcagcatcatcttggccctgctacagcgaaagaactacagagtgcgaggtgagccagtgccagagccaaggccagactcaggcagggactggagtgtggagctgcaggagctgcctgagctgtgtttctcccaggtaatccgggaaggaggtcatgcagtggtttgggccgggcagctgcaaggaaaactggttgccatcaaggccttcccaccgaggtctgtggctcagttccaagctgagagagcattgtacgaacttccaggcctacagcacgaccacattgtccgatttatcactgccagccgggggggtcctggccgcctgctctctgggcccctgctggtactggaactgcatcccaagggctccctgtgccactacttgacccagtacaccagtgactggggaagttccctgcggatggcactgtccctggcccagggcctggcatttctccatgaggagcgctggcagaatggccaatataaaccaggtattgcccaccgagatctgagcagccagaatgtgctcattcgggaagatggatcgtgtgccattggagacctgggccttgccttggtgctccctggcctcactcagccccctgcctggacccctactcaaccacaaggcccagctgccatcatggaagaccctgatgggctgagggagctcctagaagactgttgggatgcagacccagaagcacggctgacagctgagtgtgtacagcagcgcctggctgccttggcccatcctcaagagagccacccctttccagagagctgtccacgtggctgcccacctctctgcccagaagactgtacttcaattcctgcccctaccatcctcccctgtaggcctcagcggagtgcctgccacttcagcgttcagcaaggcccttgttccaggaatcctcagcctgcctgtaccctttctcctgtg

SEQ ID NO: 82 - DNA, 396 bp, Homo sapiens; identical to SEQ ID NO: 53
  source 1..396: mol_type = unassigned DNA

SEQ ID NO: 83 - protein, 546 aa, Homo sapiens
MAESAGASSFFPLVVLLLAGSGGSGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVELVGIIAGPVFLLFLIIIIVFLVINYHQRVYHNRQRLDMEDPSCEMCLSKDKTLQDLVYDLSTSGSGSGLPLFVQRTVARTIVLQEIIGKGRFGEVWRGRWRGGDVAVKIFSSREERSWFREAEIYQTVMLRHENILGFIAADNKADCSFLTLPWEVVMVSAAPKLRSLRLQYKGGRGRARFLFPLNNGTWTQLWLVSDYHEHGSLFDYLNRYTVTIEGMIKLALSAASGLAHLHMEIVGTQGKPGIAHRDLKSKNILVKKNGMCAIADLGLAVRHDAVTDTIDIAPNQRVGTKRYMAPEVLDETINMKHFDSFKCADIYALGLVYWEIARRCNSGGVHEEYQLPYYDLVPSDPSIEEMRKVVCDQKLRPNIPNWWQSYEALRVMGKMMRECWYANGAARLTALRIKKTLSQLSVQEDVKI

SEQ ID NO: 84 - protein, 103 aa, Homo sapiens; identical to SEQ ID NO: 27

SEQ ID NO: 85 - DNA, 1638 bp, Homo sapiens
  source 1..1638: mol_type = unassigned DNA
atggcggagtcggccggagcctcctccttcttcccccttgttgtcctcctgctcgccggcagcggcgggtccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtaggcatcatcgccggcccggtgttcctcctgttcctcatcatcatcattgttttccttgtcattaactatcatcagcgtgtctatcacaaccgccagagactggacatggaagatccctcatgtgagatgtgtctctccaaagacaagacgctccaggatcttgtctacgatctctccacctcagggtctggctcagggttacccctctttgtccagcgcacagtggcccgaaccatcgttttacaagagattattggcaagggtcggtttggggaagtatggcggggccgctggaggggtggtgatgtggctgtgaaaatattctcttctcgtgaagaacggtcttggttcagggaagcagagatataccagacggtcatgctgcgccatgaaaacatccttggatttattgctgctgacaataaagcagactgctcattcctcacattgccatgggaagttgtaatggtctctgctgcccccaagctgaggagccttagactccaatacaagggaggaaggggaagagcaagatttttattcccactgaataatggcacctggacacagctgtggcttgtttctgactatcatgagcacgggtccctgtttgattatctgaaccggtacacagtgacaattgaggggatgattaagctggccttgtctgctgctagtgggctggcacacctgcacatggagatcgtgggcacccaagggaagcctggaattgctcatcgagacttaaagtcaaagaacattctggtgaagaaaaatggcatgtgtgccatagcagacctgggcctggctgtccgtcatgatgcagtcactgacaccattgacattgccccgaatcagagggtggggaccaaacgatacatggcccctgaagtacttgatgaaaccattaatatgaaacactttgactcctttaaatgtgctgatatttatgccctcgggcttgtatattgggagattgctcgaagatgcaattctggaggagtccatgaagaatatcagctgccatattacgacttagtgccctctgacccttccattgaggaaatgcgaaaggttgtatgtgatcagaagctgcgtcccaacatccccaactggtggcagagttatgaggcactgcgggtgatggggaagatgatgcgagagtgttggtatgccaacggcgcagcccgcctgacggccctgcgcatcaagaagaccctctcccagctcagcgtgcaggaagacgtgaagatc

SEQ ID NO: 86 - DNA, 309 bp, Homo sapiens; identical to SEQ ID NO: 29
  source 1..309: mol_type = unassigned DNA

SEQ ID NO: 87 - protein, 507 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 88 - protein, 106 aa, Homo sapiens
AALLPGATALQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVEL

SEQ ID NO: 89 - DNA, 1521 bp, Homo sapiens
  source 1..1521: mol_type = unassigned DNA
atggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactggccctttttcagtaaagtcatcacctggccttggtcctgtggaactggcagctgtcattgctggaccagtgtgcttcgtctgcatctcactcatgttgatggtctatatctgccacaaccgcactgtcattcaccatcgagtgccaaatgaagaggacccttcattagatcgcccttttatttcagagggtactacgttgaaagacttaatttatgatatgacaacgtcaggttctggctcaggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatg

SEQ ID NO: 90 - DNA, 318 bp, Homo sapiens
  source 1..318: mol_type = unassigned DNA
gcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactggccctttttcagtaaagtcatcacctggccttggtcctgtggaactg

SEQ ID NO: 91 - protein, 532 aa, Homo sapiens
MGWLEELNWQLHIFLLILLSMHTRANFLDNMLLRSAGKLNVGTKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRALLISVTVCSLLLVLIILFCYFRYKRQETRPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDAKSMLKLAYSSVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKL

SEQ ID NO: 92 - protein, 131 aa, Homo sapiens
NFLDNMLLRSAGKLNVGTKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHR

SEQ ID NO: 93 - DNA, 1596 bp, Homo sapiens
  source 1..1596: mol_type = unassigned DNA
atgggttggctggaagaactaaactggcagcttcacattttcttgctcattcttctctctatgcacacaagggcaaacttccttgataacatgcttttgcgaagtgcaggaaaattaaatgtgggcaccaagaaagaggatggtgagagtacagcccccaccccccgtccaaaggtcttgcgttgtaaatgccaccaccattgtccagaagactcagtcaacaatatttgcagcacagacggatattgtttcacgatgatagaagaggatgactctgggttgcctgtggtcacttctggttgcctaggactagaaggctcagattttcagtgtcgggacactcccattcctcatcaaagaagatcaattgaatgctgcacagaaaggaacgaatgtaataaagacctacaccctacactgcctccattgaaaaacagagattttgttgatggacctatacaccacagggctttacttatatctgtgactgtctgtagtttgctcttggtccttatcatattattttgttacttccggtataaaagacaagaaaccagacctcgatacagcattgggttagaacaggatgaaacttacattcctcctggagaatccctgagagacttaattgagcagtctcagagctcaggaagtggatcaggcctccctctgctggtccaaaggactatagctaagcagattcagatggtgaaacagattggaaaaggtcgctatggggaagtttggatgggaaagtggcgtggcgaaaaggtagctgtgaaagtgttcttcaccacagaggaagccagctggttcagagagacagaaatatatcagacagtgttgatgaggcatgaaaacattttgggtttcattgctgcagatatcaaagggacagggtcctggacccagttgtacctaatcacagactatcatgaaaatggttccctttatgattatctgaagtccaccaccctagacgctaaatcaatgctgaagttagcctactcttctgtcagtggcttatgtcatttacacacagaaatctttagtactcaaggcaaaccagcaattgcccatcgagatctgaaaagtaaaaacattctggtgaagaaaaatggaacttgctgtattgctgacctgggcctggctgttaaatttattagtgatacaaatgaagttgacataccacctaacactcgagttggcaccaaacgctatatgcctccagaagtgttggacgagagcttgaacagaaatcacttccagtcttacatcatggctgacatgtatagttttggcctcatcctttgggaggttgctaggagatgtgtatcaggaggtatagtggaagaataccagcttccttatcatgacctagtgcccagtgacccctcttatgaggacatgagggagattgtgtgcatcaagaagttacgcccctcattcccaaaccggtggagcagtgatgagtgtctaaggcagatgggaaaactcatgacagaatgctgggctcacaatcctgcatcaaggctgacagccctgcgggttaagaaaacacttgccaaaatgtcagagtcccaggacattaaactc

SEQ ID NO: 94 - DNA, 393 bp, Homo sapiens
  source 1..393: mol_type = unassigned DNA
aacttccttgataacatgcttttgcgaagtgcaggaaaattaaatgtgggcaccaagaaagaggatggtgagagtacagcccccaccccccgtccaaaggtcttgcgttgtaaatgccaccaccattgtccagaagactcagtcaacaatatttgcagcacagacggatattgtttcacgatgatagaagaggatgactctgggttgcctgtggtcacttctggttgcctaggactagaaggctcagattttcagtgtcgggacactcccattcctcatcaaagaagatcaattgaatgctgcacagaaaggaacgaatgtaataaagacctacaccctacactgcctccattgaaaaacagagattttgttgatggacctatacaccacagg

SEQ ID NO: 95 - protein, 36 aa, Homo sapiens
GRCKIRHIGSNNRLQRSTCQNTGWESAHVMKTPGFR

SEQ ID NO: 96-97 - skipped (2 entries)

SEQ ID NO: 98 - protein, 22 aa, synthetic construct
  REGION 1..22: note = Description of Artificial Sequence: Synthetic peptide
MDAMKRGLCCVLLLCGAVFVSP

SEQ ID NO: 99 - skipped

SEQ ID NO: 100 - protein, 368 aa, synthetic construct
  REGION 1..368: note = Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 101 - DNA, 1104 bp, synthetic construct
  misc_feature 1..1104: note = Description of Artificial Sequence: Synthetic polynucleotide
  source 1..1104: mol_type = other DNA
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 102 - protein, 343 aa, synthetic construct
  REGION 1..343: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 103 - skipped

SEQ ID NO: 104 - protein, 355 aa, synthetic construct
  REGION 1..355: note = Description of Artificial Sequence: Synthetic polypeptide
MDAMKRGLCCVLLLCGAVFVSPGASGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 105 - DNA, 1065 bp, synthetic construct
  misc_feature 1..1065: note = Description of Artificial Sequence: Synthetic polynucleotide
  source 1..1065: mol_type = other DNA
atggatgcaatgaagagagggctctgctgtgtgctgctgctgtgtggagcagtcttcgtttcgcccggcgcctccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 106 - protein, 331 aa, synthetic construct
  REGION 1..331: note = Description of Artificial Sequence: Synthetic polypeptide
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 107-111 - skipped (5 entries)

SEQ ID NO: 112 - protein, 341 aa, synthetic construct
  REGION 1..341: note = Description of Artificial Sequence: Synthetic polypeptide
MDAMKRGLCCVLLLCGAVFVSPGAGLKCVCLLCDSSNFTCQTEGACWASVMLTNGKEQVIKSCVSLPELNAQVFCHSSNNVTKTECCFTDFCNNITLHLPTASPNAPKLGPMETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 113 - DNA, 1023 bp, synthetic construct
  misc_feature 1..1023: note = Description of Artificial Sequence: Synthetic polynucleotide
  source 1..1023: mol_type = other DNA
atggatgcaatgaagagagggctctgctgtgtgctgctgctgtgtggagcagtcttcgtttcgcccggcgccggactgaagtgtgtatgtcttttgtgtgattcttcaaactttacctgccaaacagaaggagcatgttgggcatcagtcatgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacagcatcaccaaatgccccaaaacttggacccatggagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 114 - protein, 317 aa, synthetic construct
  REGION 1..317: note = Description of Artificial Sequence: Synthetic polypeptide
GLKCVCLLCDSSNFTCQTEGACWASVMLTNGKEQVIKSCVSLPELNAQVFCHSSNNVTKTECCFTDFCNNITLHLPTASPNAPKLGPMETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 115 - protein, 381 aa, synthetic construct
  REGION 1..381: note = Description of Artificial Sequence: Synthetic polypeptide
MDAMKRGLCCVLLLCGAVFVSPGAQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 116 - DNA, 1143 bp, synthetic construct
  misc_feature 1..1143: note = Description of Artificial Sequence: Synthetic polynucleotide
  source 1..1143: mol_type = other DNA
atggatgcaatgaagagagggctctgctgtgtgctgctgctgtgtggagcagtcttcgtttcgcccggcgcccagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgaaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 117 - protein, 359 aa, synthetic construct
  REGION 1..359: note = Description of Artificial Sequence: Synthetic polypeptide
GAQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 118 - protein, 369 aa, synthetic construct
  REGION 1..369: note = Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 119 - DNA, 1107 bp, synthetic construct
  misc_feature 1..1107: note = Description of Artificial Sequence: Synthetic polynucleotide
  source 1..1107: mol_type = other DNA
atggatgcaatgaagagagggctctgctgtgtgctgctgctgtgtggagcagtcttcgtttcgcccggcgccgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 120 - protein, 344 aa, synthetic construct
  REGION 1..344: note = Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 121 - protein, 376 aa, synthetic construct
  REGION 1..376: note = Description of Artificial Sequence: Synthetic polypeptide
MDAMKRGLCCVLLLCGAVFVSPGASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 122 - DNA, 1128 bp, synthetic construct
  misc_feature 1..1128: note = Description of Artificial Sequence: Synthetic polynucleotide
  source 1..1128: mol_type = other DNA
atggatgcaatgaagagagggctctgctgtgtgctgctgctgtgtggagcagtcttcgtttcgcccggcgcctcgcagaatcaagaacgcctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 123 - protein, 352 aa, synthetic construct
  REGION 1..352: note = Description of Artificial Sequence: Synthetic polypeptide
SQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 124 - protein, 351 aa, synthetic construct
  REGION 1..351: note = Description of Artificial Sequence: Synthetic polypeptide
MDAMKRGLCCVLLLCGAVFVSPGADPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 125 - DNA, 1053 bp, synthetic construct
  misc_feature 1..1053: note = Description of Artificial Sequence: Synthetic polynucleotide
  source 1..1053: mol_type = other DNA
atggatgcaatgaagagagggctctgctgtgtgctgctgctgtgtggagcagtcttcgtttcgcccggcgccgaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcaggggccgccccaccgagttcgtcaaccactactgctgcgacagccacctctgcaaccacaacgtgtccctggtgctggaggccacccaacctccttcggagcagccgggaacagatggccagctggccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 126 - protein, 327 aa, synthetic construct
  REGION 1..327: note = Description of Artificial Sequence: Synthetic polypeptide
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 127 - protein, 390 aa, synthetic construct
  REGION 1..390: note = Description of Artificial Sequence: Synthetic polypeptide
MDAMKRGLCCVLLLCGAVFVSPGATIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 128 - DNA, 1170 bp, synthetic construct
  misc_feature 1..1170: note = Description of Artificial Sequence: Synthetic polynucleotide
  source 1..1170: mol_type = other DNA
atggatgcaatgaagagagggctctgctgtgtgctgctgctgtgtggagcagtcttcgtttcgcccggcgccacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 129 - protein, 366 aa, synthetic construct
  REGION 1..366: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 130 - protein, 415 aa, synthetic construct
  REGION 1..415: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 131 - DNA, 1245 bp, synthetic construct
  misc_feature 1..1245: note = Description of Artificial Sequence: Synthetic polynucleotide
  source 1..1245: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 132 - protein, 391 aa, synthetic construct
  REGION 1..391: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 133 - protein, 385 aa, synthetic construct
  REGION 1..385: note = Description of Artificial Sequence: Synthetic polypeptide
MDAMKRGLCCVLLLCGAVFVSPGAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 134 - skipped

SEQ ID NO: 135 - protein, 361 aa, synthetic construct
  REGION 1..361: note = Description of Artificial Sequence: Synthetic polypeptide
PPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 136 - protein, 355 aa, synthetic construct
  REGION 1..355: note = Description of Artificial Sequence: Synthetic polypeptide
MDAMKRGLCCVLLLCGAVFVSPGAMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 137 - DNA, 1065 bp, synthetic construct
  misc_feature 1..1065: note = Description of Artificial Sequence: Synthetic polynucleotide
  source 1..1065: mol_type = other DNA
atggatgcaatgaagagagggctctgctgtgtgctgctgctgtgtggagcagtcttcgtttcgcccggcgccatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagctgtggagtgctgccaaggggactggtgtaacaggaacatcacggcccagctgcccactaaaggaaaatccttccctggaacacagaatttccacttggagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 138 - protein, 331 aa, synthetic construct
  REGION 1..331: note = Description of Artificial Sequence: Synthetic polypeptide
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 139 - protein, 352 aa, synthetic construct
  REGION 1..352: note = Description of Artificial Sequence: Synthetic polypeptide
MDAMKRGLCCVLLLCGAVFVSPGAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 140 - DNA, 1056 bp, synthetic construct
  misc_feature 1..1056: note = Description of Artificial Sequence: Synthetic polynucleotide
  source 1..1056: mol_type = other DNA
atggatgcaatgaagagagggctctgctgtgtgctgctgctgtgtggagcagtcttcgtttcgcccggcgccgcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaaaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 141 - protein, 328 aa, synthetic construct
  REGION 1..328: note = Description of Artificial Sequence: Synthetic polypeptide
ALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 142 - protein, 365 aa, synthetic construct
  REGION 1..365: note = Description of Artificial Sequence: Synthetic polypeptide
MDAMKRGLCCVLLLCGAVFVSPGAKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 143 - DNA, 1095 bp, synthetic construct
  misc_feature 1..1095: note = Description of Artificial Sequence: Synthetic polynucleotide
  source 1..1095: mol_type = other DNA
atggatgcaatgaagagagggctctgctgtgtgctgctgctgtgtggagcagtcttcgtttcgcccggcgccaagaaagaggatggtgagagtacagcccccaccccccgtccaaaggtcttgcgttgtaaatgccaccaccattgtccagaagactcagtcaacaatatttgcagcacagacggatattgtttcacgatgatagaagaggatgactctgggttgcctgtggtcacttctggttgcctaggactagaaggctcagattttcagtgtcgggacactcccattcctcatcaaagaagatcaattgaatgctgcacagaaaggaacgaatgtaataaagacctacaccctacactgcctccattgaaaaacagagattttgttgatggacctatacaccacaggaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 144 - protein, 341 aa, synthetic construct
  REGION 1..341: note = Description of Artificial Sequence: Synthetic polypeptide
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 145-150 - skipped (6 entries)

SEQ ID NO: 151 - protein, 369 aa, synthetic construct
  REGION 1..369: note = Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 152 - protein, 344 aa, synthetic construct
  REGION 1..344: note = Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 153 - protein, 368 aa, synthetic construct
  REGION 1..368: note = Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 154 - protein, 343 aa, synthetic construct
  REGION 1..343: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 155 - protein, 376 aa, synthetic construct
  REGION 1..376: note = Description of Artificial Sequence: Synthetic polypeptide
MDAMKRGLCCVLLLCGAVFVSPGASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 156 - protein, 352 aa, synthetic construct
  REGION 1..352: note = Description of Artificial Sequence: Synthetic polypeptide
SQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 157 - protein, 390 aa, synthetic construct
  REGION 1..390: note = Description of Artificial Sequence: Synthetic polypeptide
MDAMKRGLCCVLLLCGAVFVSPGATIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 158 - protein, 366 aa, synthetic construct
  REGION 1..366: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 159 - protein, 415 aa, synthetic construct
  REGION 1..415: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 160 - protein, 391 aa, synthetic construct
  REGION 1..391: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 161 - protein, 385 aa, synthetic construct
  REGION 1..385: note = Description of Artificial Sequence: Synthetic polypeptide
MDAMKRGLCCVLLLCGAVFVSPGAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 162 - protein, 361 aa, synthetic construct
  REGION 1..361: note = Description of Artificial Sequence: Synthetic polypeptide
PPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 163-170 - skipped (8 entries)

SEQ ID NO: 171 - protein, 351 aa, synthetic construct
  REGION 1..351: note = Description of Artificial Sequence: Synthetic polypeptide
MDAMKRGLCCVLLLCGAVFVSPGADPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 172 - protein, 327 aa, synthetic construct
  REGION 1..327: note = Description of Artificial Sequence: Synthetic polypeptide
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 173 - protein, 355 aa, synthetic construct
  REGION 1..355: note = Description of Artificial Sequence: Synthetic polypeptide
MDAMKRGLCCVLLLCGAVFVSPGAMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 174 - protein, 331 aa, synthetic construct
  REGION 1..331: note = Description of Artificial Sequence: Synthetic polypeptide
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 175 - protein, 381 aa, synthetic construct
  REGION 1..381: note = Description of Artificial Sequence: Synthetic polypeptide
MDAMKRGLCCVLLLCGAVFVSPGAQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 176 - protein, 359 aa, synthetic construct
  REGION 1..359: note = Description of Artificial Sequence: Synthetic polypeptide
GAQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 177 - protein, 355 aa, synthetic construct
  REGION 1..355: note = Description of Artificial Sequence: Synthetic polypeptide
MDAMKRGLCCVLLLCGAVFVSPGASGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 178 - protein, 331 aa, synthetic construct
  REGION 1..331: note = Description of Artificial Sequence: Synthetic polypeptide
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 179 - protein, 352 aa, synthetic construct
  REGION 1..352: note = Description of Artificial Sequence: Synthetic polypeptide
MDAMKRGLCCVLLLCGAVFVSPGAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 180 - protein, 328 aa, synthetic construct
  REGION 1..328: note = Description of Artificial Sequence: Synthetic polypeptide
ALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 181 - protein, 365 aa, synthetic construct
  REGION 1..365: note = Description of Artificial Sequence: Synthetic polypeptide
MDAMKRGLCCVLLLCGAVFVSPGAKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 182 - protein, 341 aa, synthetic construct
  REGION 1..341: note = Description of Artificial Sequence: Synthetic polypeptide
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 183 - protein, 341 aa, synthetic construct
  REGION 1..341: note = Description of Artificial Sequence: Synthetic polypeptide
MDAMKRGLCCVLLLCGAVFVSPGAGLKCVCLLCDSSNFTCQTEGACWASVMLTNGKEQVIKSCVSLPELNAQVFCHSSNNVTKTECCFTDFCNNITLHLPTASPNAPKLGPMETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 184 - protein, 317 aa, synthetic construct
  REGION 1..317: note = Description of Artificial Sequence: Synthetic polypeptide
GLKCVCLLCDSSNFTCQTEGACWASVMLTNGKEQVIKSCVSLPELNAQVFCHSSNNVTKTECCFTDFCNNITLHLPTASPNAPKLGPMETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 185-199 - skipped (15 entries)

SEQ ID NO: 200 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 201 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 202 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 204 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 206 - protein, 228 aa, synthetic construct
  REGION 1..228: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 207 - protein, 228 aa, synthetic construct
  REGION 1..228: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWESNGQELPREKYLTWAPVLDSDGSFFLYSILRVAAEDWKKGDTFSCSVMHEALHNHYTQKSLDRSPGK

SEQ ID NO: 208 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 210 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 211 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 212 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - protein, 261 aa, synthetic construct
  REGION 1..261: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLEKELQALEKENAQLEWELQALEKELAQGAT

SEQ ID NO: 214 - protein, 261 aa, synthetic construct
  REGION 1..261: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLKKKLQALKKKNAQLKWKLQALKKKLAQGAT

SEQ ID NO: 215-300 - skipped (86 entries)

SEQ ID NO: 301 - protein, 443 aa, Homo sapiens
MLTNGKEQVIKSCVSLPELNAQVFCHSSNNVTKTECCFTDFCNNITLHLPTASPNAPKLGPMELAIIITVPVCLLSIAAMLTVWACQGRQCSYRKKKRPNVEEPLSECNLVNAGKTLKDLIYDVTASGSGSGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 302 - protein, 63 aa, Homo sapiens
MLTNGKEQVIKSCVSLPELNAQVFCHSSNNVTKTECCFTDFCNNITLHLPTASPNAPKLGPME

SEQ ID NO: 303 - DNA, 1329 bp, Homo sapiens
  source 1..1329: mol_type = unassigned DNA
atgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacagcatcaccaaatgccccaaaacttggacccatggagctggccatcattattactgtgcctgtttgcctcctgtccatagctgcgatgctgacagtatgggcatgccagggtcgacagtgctcctacaggaagaaaaagagaccaaatgtggaggaaccactctctgagtgcaatctggtaaatgctggaaaaactctgaaagatctgatttatgatgtgaccgcctctggatctggctctggtctacctctgttggttcaaaggacaattgcaaggacgattgtgcttcaggaaatagtaggaaaaggtagatttggtgaggtgtggcatggaagatggtgtggggaagatgtggctgtgaaaatattctcctccagagatgaaagatcttggtttcgtgaggcagaaatttaccagacggtcatgctgcgacatgaaaacatccttggtttcattgctgctgacaacaaagataatggaacttggactcaactttggctggtatctgaatatcatgaacagggctccttatatgactatttgaatagaaatatagtgaccgtggctggaatgatcaagctggcgctctcaattgctagtggtctggcacaccttcatatggagattgttggtacacaaggtaaacctgctattgctcatcgagacataaaatcaaagaatatcttagtgaaaaagtgtgaaacttgtgccatagcggacttagggttggctgtgaagcatgattcaatactgaacactatcgacatacctcagaatcctaaagtgggaaccaagaggtatatggctcctgaaatgcttgatgatacaatgaatgtgaatatctttgagtccttcaaacgagctgacatctattctgttggtctggtttactgggaaatagcccggaggtgttcagtcggaggaattgttgaggagtaccaattgccttattatgacatggtgccttcagatccctcgatagaggaaatgagaaaggttgtttgtgaccagaagtttcgaccaagtatcccaaaccagtggcaaagttgtgaagcactccgagtcatggggagaataatgcgtgagtgttggtatgccaacggagcggcccgcctaactgctcttcgtattaagaagactatatctcaactttgtgtcaaagaagactgcaaagcc

SEQ ID NO: 304 - DNA, 189 bp, Homo sapiens
  source 1..189: mol_type = unassigned DNA
atgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacagcatcaccaaatgccccaaaacttggacccatggag

SEQ ID NO: 305 - protein, 413 aa, Homo sapiens
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 306 - protein, 393 aa, Homo sapiens
ELSPGLKCVCLLCDSSNFTCQTEGACWASVMLTNGKEQVIKSCVSLPELNAQVFCHSSNNVTKTECCFTDFCNNITLHLPTGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 307 - DNA, 1239 bp, Homo sapiens
  source 1..1239: mol_type = unassigned DNA
atgacccgggcgctctgctcagcgctccgccaggctctcctgctgctcgcagcggccgccgagctctcgccaggactgaagtgtgtatgtcttttgtgtgattcttcaaactttacctgccaaacagaaggagcatgttgggcatcagtcatgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacaggtctacctctgttggttcaaaggacaattgcaaggacgattgtgcttcaggaaatagtaggaaaaggtagatttggtgaggtgtggcatggaagatggtgtggggaagatgtggctgtgaaaatattctcctccagagatgaaagatcttggtttcgtgaggcagaaatttaccagacggtcatgctgcgacatgaaaacatccttggtttcattgctgctgacaacaaagataatggaacttggactcaactttggctggtatctgaatatcatgaacagggctccttatatgactatttgaatagaaatatagtgaccgtggctggaatgatcaagctggcgctctcaattgctagtggtctggcacaccttcatatggagattgttggtacacaaggtaaacctgctattgctcatcgagacataaaatcaaagaatatcttagtgaaaaagtgtgaaacttgtgccatagcggacttagggttggctgtgaagcatgattcaatactgaacactatcgacatacctcagaatcctaaagtgggaaccaagaggtatatggctcctgaaatgcttgatgatacaatgaatgtgaatatctttgagtccttcaaacgagctgacatctattctgttggtctggtttactgggaaatagcccggaggtgttcagtcggaggaattgttgaggagtaccaattgccttattatgacatggtgccttcagatccctcgatagaggaaatgagaaaggttgtttgtgaccagaagtttcgaccaagtatcccaaaccagtggcaaagttgtgaagcactccgagtcatggggagaataatgcgtgagtgttggtatgccaacggagcggcccgcctaactgctcttcgtattaagaagactatatctcaactttgtgtcaaagaagactgcaaagcc

SEQ ID NO: 308 - DNA, 1179 bp, Homo sapiens
  source 1..1179: mol_type = unassigned DNA
gagctctcgccaggactgaagtgtgtatgtcttttgtgtgattcttcaaactttacctgccaaacagaaggagcatgttgggcatcagtcatgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacaggtctacctctgttggttcaaaggacaattgcaaggacgattgtgcttcaggaaatagtaggaaaaggtagatttggtgaggtgtggcatggaagatggtgtggggaagatgtggctgtgaaaatattctcctccagagatgaaagatcttggtttcgtgaggcagaaatttaccagacggtcatgctgcgacatgaaaacatccttggtttcattgctgctgacaacaaagataatggaacttggactcaactttggctggtatctgaatatcatgaacagggctccttatatgactatttgaatagaaatatagtgaccgtggctggaatgatcaagctggcgctctcaattgctagtggtctggcacaccttcatatggagattgttggtacacaaggtaaacctgctattgctcatcgagacataaaatcaaagaatatcttagtgaaaaagtgtgaaacttgtgccatagcggacttagggttggctgtgaagcatgattcaatactgaacactatcgacatacctcagaatcctaaagtgggaaccaagaggtatatggctcctgaaatgcttgatgatacaatgaatgtgaatatctttgagtccttcaaacgagctgacatctattctgttggtctggtttactgggaaatagcccggaggtgttcagtcggaggaattgttgaggagtaccaattgccttattatgacatggtgccttcagatccctcgatagaggaaatgagaaaggttgtttgtgaccagaagtttcgaccaagtatcccaaaccagtggcaaagttgtgaagcactccgagtcatggggagaataatgcgtgagtgttggtatgccaacggagcggcccgcctaactgctcttcgtattaagaagactatatctcaactttgtgtcaaagaagactgcaaagcc

SEQ ID NO: 309 - protein, 336 aa, Homo sapiens
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 310 - protein, 316 aa, Homo sapiens
ELSPGLKCVCLLCDSSNFTCQTEGACWASVMLTNGKEQVIKSCVSLPELNAQVFCHSSNNVTKTECCFTDFCNNITLHLPTDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 311 - DNA, 1011 bp, Homo sapiens
  source 1..1011: mol_type = unassigned DNA
atgacccgggcgctctgctcagcgctccgccaggctctcctgctgctcgcagcggccgccgagctctcgccaggactgaagtgtgtatgtcttttgtgtgattcttcaaactttacctgccaaacagaaggagcatgttgggcatcagtcatgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacagataatggaacttggactcaactttggctggtatctgaatatcatgaacagggctccttatatgactatttgaatagaaatatagtgaccgtggctggaatgatcaagctggcgctctcaattgctagtggtctggcacaccttcatatggagattgttggtacacaaggtaaacctgctattgctcatcgagacataaaatcaaagaatatcttagtgaaaaagtgtgaaacttgtgccatagcggacttagggttggctgtgaagcatgattcaatactgaacactatcgacatacctcagaatcctaaagtgggaaccaagaggtatatggctcctgaaatgcttgatgatacaatgaatgtgaatatctttgagtccttcaaacgagctgacatctattctgttggtctggtttactgggaaatagcccggaggtgttcagtcggaggaattgttgaggagtaccaattgccttattatgacatggtgccttcagatccctcgatagaggaaatgagaaaggttgtttgtgaccagaagtttcgaccaagtatcccaaaccagtggcaaagttgtgaagcactccgagtcatggggagaataatgcgtgagtgttggtatgccaacggagcggcccgcctaactgctcttcgtattaagaagactatatctcaactttgtgtcaaagaagactgcaaagcctaa

SEQ ID NO: 312 - DNA, 951 bp, Homo sapiens
  source 1..951: mol_type = unassigned DNA
gagctctcgccaggactgaagtgtgtatgtcttttgtgtgattcttcaaactttacctgccaaacagaaggagcatgttgggcatcagtcatgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacagataatggaacttggactcaactttggctggtatctgaatatcatgaacagggctccttatatgactatttgaatagaaatatagtgaccgtggctggaatgatcaagctggcgctctcaattgctagtggtctggcacaccttcatatggagattgttggtacacaaggtaaacctgctattgctcatcgagacataaaatcaaagaatatcttagtgaaaaagtgtgaaacttgtgccatagcggacttagggttggctgtgaagcatgattcaatactgaacactatcgacatacctcagaatcctaaagtgggaaccaagaggtatatggctcctgaaatgcttgatgatacaatgaatgtgaatatctttgagtccttcaaacgagctgacatctattctgttggtctggtttactgggaaatagcccggaggtgttcagtcggaggaattgttgaggagtaccaattgccttattatgacatggtgccttcagatccctcgatagaggaaatgagaaaggttgtttgtgaccagaagtttcgaccaagtatcccaaaccagtggcaaagttgtgaagcactccgagtcatggggagaataatgcgtgagtgttggtatgccaacggagcggcccgcctaactgctcttcgtattaagaagactatatctcaactttgtgtcaaagaagactgcaaagcctaa

SEQ ID NO: 313 - protein, 88 aa, Homo sapiens
LKCVCLLCDSSNFTCQTEGACWASVMLTNGKEQVIKSCVSLPELNAQVFCHSSNNVTKTECCFTDFCNNITLHLPTASPNAPKLGPME

SEQ ID NO: 314-400 - skipped (87 entries)

SEQ ID NO: 401 - protein, 368 aa, synthetic construct
  REGION 1..368: note = Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 402 - protein, 343 aa, synthetic construct
  REGION 1..343: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 403 - protein, 356 aa, synthetic construct
  REGION 1..356: note = Description of Artificial Sequence: Synthetic polypeptide
MDAMKRGLCCVLLLCGAVFVSPGASGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 404 - protein, 332 aa, synthetic construct
  REGION 1..332: note = Description of Artificial Sequence: Synthetic polypeptide
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 405 - protein, 342 aa, synthetic construct
  REGION 1..342: note = Description of Artificial Sequence: Synthetic polypeptide
MDAMKRGLCCVLLLCGAVFVSPGAGLKCVCLLCDSSNFTCQTEGACWASVMLTNGKEQVIKSCVSLPELNAQVFCHSSNNVTKTECCFTDFCNNITLHLPTASPNAPKLGPMETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 406 - protein, 318 aa, synthetic construct
  REGION 1..318: note = Description of Artificial Sequence: Synthetic polypeptide
GLKCVCLLCDSSNFTCQTEGACWASVMLTNGKEQVIKSCVSLPELNAQVFCHSSNNVTKTECCFTDFCNNITLHLPTASPNAPKLGPMETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 407 - protein, 382 aa, synthetic construct
  REGION 1..382: note = Description of Artificial Sequence: Synthetic polypeptide
MDAMKRGLCCVLLLCGAVFVSPGAQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 408 - protein, 360 aa, synthetic construct
  REGION 1..360: note = Description of Artificial Sequence: Synthetic polypeptide
GAQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 409 - protein, 369 aa, synthetic construct
  REGION 1..369: note = Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 410 - protein, 344 aa, synthetic construct
  REGION 1..344: note = Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 411 - protein, 376 aa, synthetic construct
  REGION 1..376: note = Description of Artificial Sequence: Synthetic polypeptide
MDAMKRGLCCVLLLCGAVFVSPGASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 412 - protein, 352 aa, synthetic construct
  REGION 1..352: note = Description of Artificial Sequence: Synthetic polypeptide
SQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 413 - protein, 352 aa, synthetic construct
  REGION 1..352: note = Description of Artificial Sequence: Synthetic polypeptide
MDAMKRGLCCVLLLCGAVFVSPGADPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 414 - protein, 328 aa, synthetic construct
  REGION 1..328: note = Description of Artificial Sequence: Synthetic polypeptide
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 415 - protein, 390 aa, synthetic construct
  REGION 1..390: note = Description of Artificial Sequence: Synthetic polypeptide
MDAMKRGLCCVLLLCGAVFVSPGATIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 416 - protein, 366 aa, synthetic construct
  REGION 1..366: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 417 - protein, 415 aa, synthetic construct
  REGION 1..415: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 418 - protein, 391 aa, synthetic construct
  REGION 1..391: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 419 - protein, 385 aa, synthetic construct
  REGION 1..385: note = Description of Artificial Sequence: Synthetic polypeptide
MDAMKRGLCCVLLLCGAVFVSPGAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 420 - protein, 361 aa, synthetic construct
  REGION 1..361: note = Description of Artificial Sequence: Synthetic polypeptide
PPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 421 - protein, 356 aa, synthetic construct
  REGION 1..356: note = Description of Artificial Sequence: Synthetic polypeptide
MDAMKRGLCCVLLLCGAVFVSPGAMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 422 - protein, 332 aa, synthetic construct
  REGION 1..332: note = Description of Artificial Sequence: Synthetic polypeptide
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 423 - protein, 353 aa, synthetic construct
  REGION 1..353: note = Description of Artificial Sequence: Synthetic polypeptide
MDAMKRGLCCVLLLCGAVFVSPGAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 424 - protein, 329 aa, synthetic construct
  REGION 1..329: note = Description of Artificial Sequence: Synthetic polypeptide
ALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 425 - protein, 366 aa, synthetic construct
  REGION 1..366: note = Description of Artificial Sequence: Synthetic polypeptide
MDAMKRGLCCVLLLCGAVFVSPGAKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 426 - protein, 342 aa, synthetic construct
  REGION 1..342: note = Description of Artificial Sequence: Synthetic polypeptide
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 427-450 - skipped (24 entries)

SEQ ID NO: 451 - protein, 369 aa, synthetic construct
  REGION 1..369: note = Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 452 - protein, 344 aa, synthetic construct
  REGION 1..344: note = Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 453 - protein, 368 aa, synthetic construct
  REGION 1..368: note = Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 454 - protein, 343 aa, synthetic construct
  REGION 1..343: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 455 - protein, 376 aa, synthetic construct
  REGION 1..376: note = Description of Artificial Sequence: Synthetic polypeptide
MDAMKRGLCCVLLLCGAVFVSPGASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 456 - protein, 352 aa, synthetic construct
  REGION 1..352: note = Description of Artificial Sequence: Synthetic polypeptide
SQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 457 - protein, 385 aa, synthetic construct
  REGION 1..385: note = Description of Artificial Sequence: Synthetic polypeptide
MDAMKRGLCCVLLLCGAVFVSPGAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 458 - protein, 361 aa, synthetic construct
  REGION 1..361: note = Description of Artificial Sequence: Synthetic polypeptide
PPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 459 - protein, 390 aa, synthetic construct
  REGION 1..390: note = Description of Artificial Sequence: Synthetic polypeptide
MDAMKRGLCCVLLLCGAVFVSPGATIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 460 - protein, 366 aa, synthetic construct
  REGION 1..366: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 461 - protein, 415 aa, synthetic construct
  REGION 1..415: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 462 - protein, 391 aa, synthetic construct
  REGION 1..391: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 463 - protein, 352 aa, synthetic construct
  REGION 1..352: note = Description of Artificial Sequence: Synthetic polypeptide
MDAMKRGLCCVLLLCGAVFVSPGADPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 464 - protein, 328 aa, synthetic construct
  REGION 1..328: note = Description of Artificial Sequence: Synthetic polypeptide
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 465 - protein, 356 aa, synthetic construct
  REGION 1..356: note = Description of Artificial Sequence: Synthetic polypeptide
MDAMKRGLCCVLLLCGAVFVSPGAMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 466 - protein, 332 aa, synthetic construct
  REGION 1..332: note = Description of Artificial Sequence: Synthetic polypeptide
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 467 - protein, 382 aa, synthetic construct
  REGION 1..382: note = Description of Artificial Sequence: Synthetic polypeptide
MDAMKRGLCCVLLLCGAVFVSPGAQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 468 - protein, 360 aa, synthetic construct
  REGION 1..360: note = Description of Artificial Sequence: Synthetic polypeptide
GAQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 469 - protein, 356 aa, synthetic construct
  REGION 1..356: note = Description of Artificial Sequence: Synthetic polypeptide
MDAMKRGLCCVLLLCGAVFVSPGASGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 470 - protein, 332 aa, synthetic construct
  REGION 1..332: note = Description of Artificial Sequence: Synthetic polypeptide
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 471 - protein, 353 aa, synthetic construct
  REGION 1..353: note = Description of Artificial Sequence: Synthetic polypeptide
MDAMKRGLCCVLLLCGAVFVSPGAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 472 - protein, 329 aa, synthetic construct
  REGION 1..329: note = Description of Artificial Sequence: Synthetic polypeptide
ALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 473 - protein, 366 aa, synthetic construct
  REGION 1..366: note = Description of Artificial Sequence: Synthetic polypeptide
MDAMKRGLCCVLLLCGAVFVSPGAKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 474 - protein, 342 aa, synthetic construct
  REGION 1..342: note = Description of Artificial Sequence: Synthetic polypeptide
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 475 - protein, 342 aa, synthetic construct
  REGION 1..342: note = Description of Artificial Sequence: Synthetic polypeptide
MDAMKRGLCCVLLLCGAVFVSPGAGLKCVCLLCDSSNFTCQTEGACWASVMLTNGKEQVIKSCVSLPELNAQVFCHSSNNVTKTECCFTDFCNNITLHLPTASPNAPKLGPMETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 476 - protein, 318 aa, synthetic construct
  REGION 1..318: note = Description of Artificial Sequence: Synthetic polypeptide
GLKCVCLLCDSSNFTCQTEGACWASVMLTNGKEQVIKSCVSLPELNAQVFCHSSNNVTKTECCFTDFCNNITLHLPTASPNAPKLGPMETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 477-499 - skipped (23 entries)

SEQ ID NO: 500 - protein, 116 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPT

SEQ ID NO: 501 - protein, 150 aa, Rattus sp.
MTAPWAALALLWGSLCAGSGRGEAETRECIYYNANWELERTNQSGLERCEGEQDKRLHCYASWPNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 502 - protein, 150 aa, Sus sp.
MTAPWAALALLWGSLCVGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 503 - protein, 150 aa, Mus sp.
MTAPWAALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 504 - protein, 150 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 505 - protein, 150 aa, Bos sp.
MTAPWAALALLWGSLCAGSGRGEAETRECIYYNANWELERTNQSGLERCEGERDKRLHCYASWRNSSGTIELVKKGCWLDDFNCYDRQECVATEENPQVYFCCCEGNFCNERFTHLPEAGGPEVTYEPPPTAPTLLTVLAYSLLPVGGLS

SEQ ID NO: 506 - protein, 150 aa, Xenopus sp.
MGASVALTFLLLLATFRAGSGHDEVETRECIYYNANWELEKTNQSGVERLVEGKKDKRLHCYASWRNNSGFIELVKKGCWLDDFNCYDRQECIAKEENPQVFFCCCEGNYCNKKFTHLPEVETFDPKPQPSASVLNILIYSLLPIVGLSM

SEQ ID NO: 507 - protein, 150 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLI

SEQ ID NO: 508 - protein, 154 aa, synthetic construct
  REGION 1..154: note = Description of Artificial Sequence: Synthetic consensus polypeptide
  VARIANT 8: note = MOD_RES - Thr, Ala or not present
  VARIANT 121: note = MOD_RES - Pro, Ala, Val or Met
MTAPWAAXLALLWGSLCAGSGRGEAETRECIYYNANWELERTNQSGLERLCEGEQDKRLHCYASWRNSSGTIELVKKGCWLDDFNCYDRQECVATEENPQVYFCCCEGNFCNERFTHLPEXGGPEVTYEPKPPTAPTLLTVLAYSLLPIGGLSM

SEQ ID NO: 509 - protein, 6 aa, synthetic construct
  REGION 1..6: note = Description of Artificial Sequence: Synthetic 6xHis tag
HHHHHH